(19) United States
(12) Patent Application Publication
(10) Pub. No.: US 2024/0391882 A1
(43) Pub. Date: Nov. 28, 2024

(54) Substituted Quinoxalines

(71) Applicant: Mirati Therapeutics, Inc., San Diego, CA (US)

(72) Inventors: Matthew Arnold Marx, San Diego, CA (US); Christopher Ronald Smith, San Diego, CA (US); John Michael Ketcham, San Diego, CA (US); Stephen Harwood, San Diego, CA (US); Aaron Craig Burns, San Diego, CA (US); John David Lawson, Carlsbad, CA (US); Kersti Caddell-Haatveit, San Diego, CA (US)

(21) Appl. No.: 18/658,666

(22) Filed: May 8, 2024

Related U.S. Application Data
(60) Provisional application No. 63/501,970, filed on May 12, 2023.
(60) Provisional application No. 63/522,325, filed on Jun. 21, 2023.
(60) Provisional application No. 63/578,523, filed on Aug. 24, 2023.

(51) Int. Cl. C07D 241/44; A61K 31/498; A61K 31/4985; A61K 31/506; A61K 31/5377; A61K 31/541; A61K 31/55; A61K 31/551; A61K 31/553; C07D 401/04; C07D 401/12; C07D 401/14; C07D 403/04; C07D 403/12; C07D 405/12; C07D 405/14; C07D 409/14; C07D 413/04; C07D 413/14; C07D 417/12; C07D 417/14; C07D 471/04; C07D 471/08; C07D 487/04; C07D 487/08; C07D 487/10; C07D 491/048; C07D 491/08; C07D 491/107; C07D 491/147; C07D 491/18; C07D 495/04; C07D 495/10; C07D 498/04; C07D 498/08; C07D 498/20; C07D 513/04

(52) U.S. Cl. CPC C07D 241/44; A61K 31/498; A61K 31/4985; A61K 31/506; A61K 31/5377; A61K 31/541; A61K 31/55; A61K 31/551; A61K 31/553; C07D 401/04; C07D 401/12; C07D 401/14; C07D 403/04; C07D 403/12; C07D 405/12; C07D 405/14; C07D 409/14; C07D 413/04; C07D 413/14; C07D 417/12; C07D 417/14; C07D 471/04; C07D 471/08; C07D 487/04; C07D 487/08; C07D 487/10; C07D 491/048; C07D 491/08; C07D 491/107; C07D 491/147; C07D 491/18; C07D 495/04; C07D 495/10; C07D 498/04; C07D 498/08; C07D 498/20; C07D 513/04

(57) ABSTRACT

Disclosed herein are substituted quinoxalines, methods for their preparation, and use thereof.

### CROSS-REFERENCE TO RELATED APPLICATIONS

This application claims priority from U.S. Provisional Application No. 63/501,970, filed May 12, 2023, U.S. Provisional Application No. 63/522,325, filed Jun. 21, 2023, and U.S. Provisional Application No. 63/578,523, filed Aug. 24, 2023, the disclosure of each of which is hereby incorporated by reference in its entirety

### FIELD

This disclosure is directed to substituted quinoxalines, and more particularly to such compounds that are inhibitors of phosphoinositide 3-kinase (PI3K) and therefore useful in the treatment of diseases or disorders associated with PI3K modulation. The disclosure is directed toward compounds and compositions which inhibit PI3K, methods of treating a disease or disorder associated with PI3K (e.g., CLOVES syndrome (congenital lipomatous overgrowth, vascular malformations, epidermal naevi, scoliosis/skeletal and spinal syndrome), PIK3CA-related overgrowth syndrome (PROS), breast cancer, brain cancer, prostate cancer, endometrial cancer, gastric cancer, leukemia, lymphoma, sarcoma, colorectal cancer, lung cancer, ovarian cancer, skin cancer, or head and neck cancer), and methods of using PI3K inhibitors in combination with one or more additional disorder or cancer therapy.

### BACKGROUND

The activity of cells can be regulated by external signals that stimulate or inhibit intracellular events. The process by which stimulatory or inhibitory signals are transmitted into and within a cell to elicit an intracellular response is referred to as signal transduction. Over the past decades, cascades of signal transduction events have been elucidated and found to play a central role in a variety of biological responses. Defects in various components of signal transduction pathways have been found to account for a vast number of diseases, including numerous forms of cancer, inflammatory disorders, metabolic disorders, vascular and neuronal diseases.

Kinases represent a class of important signaling molecules. Kinases can generally be classified into protein kinases and lipid kinases, and certain kinases exhibit dual specificities. Protein kinases are enzymes that phosphorylate other proteins and/or themselves (i.e., autophosphorylation). Protein kinases can be generally classified into three major groups based upon their substrate utilization: tyrosine kinases which predominantly phosphorylate substrates okay on tyrosine residues (e.g., erb2, PDGF receptor, EGF receptor, VEGF receptor, src, abl), serine/threonine kinases which predominantly phosphorylate substrates on serine and/or threonine residues (e.g., mTORC1, mTORC2, ATM, ATR, DNA-PK, Akt), and dual-specificity kinases which phosphorylate substrates on tyrosine, serine and/or threonine residues.

Lipid kinases are enzymes that catalyze the phosphorylation of lipids within cells. These enzymes, and the resulting phosphorylated lipids and lipid-derived biologically active organic molecules, play a role in many different physiological processes, including cell proliferation, migration, adhesion, and differentiation. A particular group of lipid kinases comprises membrane lipid kinases, i.e., kinases that catalyze the phosphorylation of lipids contained in or associated with cell membranes. Examples of such enzymes include phosphoinositide(s) kinases (such as PI3-kinases, PI4-Kinases), diacylglycerol kinases, and sphingosine kinases.

The phosphoinositide 3-kinases (PI3Ks) signaling pathway is one of the most highly mutated systems in human cancers. PI3K signaling is involved in many other disease states including allergic contact dermatitis, rheumatoid arthritis, osteoarthritis, inflammatory bowel diseases, chronic obstructive pulmonary disorder, psoriasis, multiple sclerosis, asthma, disorders related to diabetic complications, and inflammatory complications of the cardiovascular system such as acute coronary syndrome.

PI3Ks are members of a unique and conserved family of intracellular lipid kinases that phosphorylate the 3′-OH group on phosphatidylinositols or phosphoinositides. The PI3K family comprises 15 kinases with distinct substrate specificities, expression patterns, and modes of regulation. The class I PI3Ks (pi 10a, pi 10b, pi 106, and pi 10 g) are typically activated by tyrosine kinases or G-protein coupled receptors to generate PIP3, which engages downstream effectors such as those in the pathways of Akt/PDKI, mTOR, the Tec family kinases, and the Rho family GTPases. The class II and III PI3-Ks play a key role in intracellular trafficking through the synthesis of PI(3)P and PI(3,4)P2.

The PI3K isoforms have been implicated, for example, in a variety of human cancers and disorders. Mutations in the gene coding for PI3K isoforms or mutations which lead to upregulation of a PI3K isoform are believed to occur in many human cancers. Mutations in the gene coding for a PI3K isoform are point mutations clustered within several hotspots in helical and kinase domains. Because of the high rate of PI3K mutations, targeting of this pathway may provide valuable therapeutic opportunities.

Genetic alterations in genes in PI3K signaling are believed to be involved in a range of cancers such as endometrial cancer, breast cancer, esophageal squamous-cell cancer, cervical squamous-cell carcinoma, cervical adenocarcinoma, colorectal adenocarcinoma, bladder urothelial carcinoma, glioblastoma, ovarian cancer, non-small-cell lung cancer, esophagogastric cancer, nerve-sheath tumor, head and neck squamous-cell carcinoma, melanoma, esophagogastric adenocarcinoma, soft-tissue sarcoma, prostate cancer, fibrolamellar carcinoma, hepatocellular carcinoma, diffuse glioma, colorectal cancer, pancreatic cancer, cholangiocarcinoma, B-cell lymphoma, mesothelioma, adrenocortical carcinoma, renal non-clear-cell carcinoma, renal clear-cell carcinoma, germ-cell carcinoma, thymic tumor, pheochromocytoma, miscellaneous neuroepithelial tumor, thyroid cancer, leukemia, and encapsulated glioma.

The alpha (a) isoform of PI3K has been implicated, for example, in a variety of human cancers. Angiogenesis has been shown to selectively require the alpha (α) isoform of PI3K in the control of endothelial cell migration. Mutations in the gene coding for PI3Kα or mutations which lead to upregulation of PI3Kα are believed to occur in many human cancers such as lung, stomach, endometrial, ovarian, bladder, breast, colon, brain, prostate, and skin cancers.

Mutations in the gene coding for PI3Kα are point mutations clustered within several hotspots in helical and kinase domains, such as E542K, E545K, and H1047R. Many of these mutations have been shown to be oncogenic gain-of-function mutations. Because of the high rate of PI3Kα mutations, targeting of this pathway may provide valuable therapeutic opportunities. While other PI3K isoforms such as PI3Kδ or PI3Kγ are expressed primarily in hematopoietic cells, PI3Kα, along with PI3Kβ, is expressed constitutively.

Due to the central role of PI3Kα in regulating organismal glucose homeostasis, PI3K inhibition in patients often gives rise to hyperglycemia and/or hyperinsulinemia. High levels of circulating insulin could potentially be mitogenic and/or antiapoptotic for cancer cells and thus negate the antiproliferative effects of PI3K inhibitors.

In the setting of cancer with mutated PI3Kα, one way to overcome the problem of compensatory production of insulin and/or glucose upon systemic PI3Kα inhibition would be to develop inhibitors with enhanced selectivity for mutant PI3Kα over wild-type PI3Kα. This would create an increased window for drug dosing to selectively inhibit the pathologic signaling of mutant PI3Kα in the cancer cells without affecting the wild-type PI3Kα in the host tissues that control systemic metabolism, thus limiting toxicities and permitting higher doses and more complete inhibition of the drug target.

Existing PI3Kα inhibitors are nearly equipotent to wild-type and mutant PI3Kα. Mutant selective inhibitors have been elusive due to the PI3Kα mutations location far from the active site. As such, inhibitors which target a second, peripheral binding pocket near a known mutation (e.g., H1047R) may provide a route to selective PI3Kα inhibition. Thus, targeting a mutated, peripheral binding pocket of PI3Kα, may in turn provide a valuable therapeutic target for drug development.

As such, kinases, for example lipid kinases such as PI3Ks, are prime targets for drug development.

### SUMMARY

In one aspect, the present disclosure provides compounds of Formula (I) and pharmaceutically acceptable salts thereof, and prodrugs, solvates, hydrates, isomers, deuterated forms, and tautomers thereof:

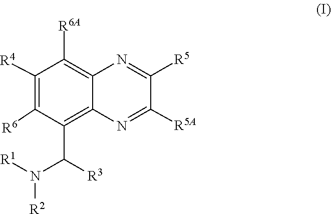

or a pharmaceutically acceptable salt or deuterated form thereof, wherein:
R¹ is H, C₁-C₃ alkyl, or C₃-C₆ cycloalkyl;
R² is phenyl or a 5-6 membered heteroaryl group, wherein each phenyl and heteroaryl is optionally substituted with 1-5 R¹²;
   each R¹² is independently C₁-C₄ alkyl, —OR^{A}, —C(O)OR^{A}, (C₁-C₃ alkyl)-OR^{A}, —C(O)N(R^{A})₂, cyano, tetrazolyl or halogen;
      each R^{A} is independently H, C₁-C₆ alkyl, C₃-C₆ cycloalkyl or 2-carboxy-3,4,5-trihydroxytetrahydro-2H-pyran-6-yl;
R³ is C₁-C₃ alkyl optionally substituted, polysubstituted or persubstituted with fluoro, C₃-C₆ cycloalkyl, C₁-C₃ alkenyl, C₂-C₃ alkynyl, or C₅-C₆ cycloalkenyl;
R⁴ is H, halogen, C₁-C₃ alkyl, C₁-C₆ alkoxy, C₃-C₆ cycloalkyl, C₁-C₃ alkenyl, C₂-C₃ alkynyl, or C₅-C₆ cycloalkenyl or cyano, wherein each C₁-C₃ alkyl and C₃-C₆ cycloalkyl is optionally substituted with 1-5 halo groups;
R⁶ and R^{6A} independently represent H or C₁-C₆ alkyl;
R⁵ is hydrogen, —OR²⁰, —SR²⁰, C₁-C₆ alkyl, halogen, hydroxy, cyano, haloC₁-C₃ alkyl, hydroxy C₁-C₆ alkyl, C₁-C₆ alkoxy, C₁-C₆ alkoxy(C₁-C₆)alkyl, cyano, amino, mono- or di(C₁-C₃) alkyl amino, C₃-C₆ cycloalkyl, C₃-C₆ cycloalkyl(C₁-C₃ alkyl), 3-8 membered heterocyclyl, phenyl or 5-6 membered heteroaryl, wherein each alkyl, alkenyl, cycloalkyl, phenyl, 3-8 membered heterocyclyl and heteroaryl is optionally independently substituted with 1-3 of halogen, hydroxy, C₁-C₃ alkyl, C₁-C₃ alkoxy, haloC₁-C₃ alkyl, hydroxy C₁-C₆ alkyl, oxo, cyano, amino, mono-ordi(C₁-C₃) alkyl amino,
   wherein each R²⁰ is aryl, aryl(C₁-C₆)alkyl, 5-7 membered heteroaryl or 5-7 membered heteroaryl(C₁-C₆)alkyl, wherein each aryl and heteroaryl is optionally independently substituted with one to four of C₁-C₆ alkyl, halogen, hydroxy, cyano, haloC₁-C₃ alkyl, hydroxy C₁-C₆ alkyl, C₁-C₆ alkoxy, C₁-C₆ alkoxy(C₁-C₆)alkyl, cyano, amino, or mono- or di(C₁-C₃) alkyl amino;
R^{5A} represents NR⁷R⁸, C₁-C₆ alkyl, C₃-C₆ cycloalkyl, C₃-C₆ cycloalkyl(C₁-C₃ alkyl), phenyl, or 5-6 membered heteroaryl, wherein each C₁-C₆ alkyl, C₃-C₆ cycloalkyl, C₃-C₆ cycloalkyl(C₁-C₃ alkyl), phenyl and heteroaryl is optionally independently substituted with 1-3 of halogen, halo(C₁-C₃ alkyl), hydroxy, oxo, C₁-C₃ alkyl, C₁-C₃ alkoxy, C₁-C₆ alkanoyl, cyano, amino, or mono- or di(C₁-C₃) alkyl amino;
R⁷ represents H or C₁-C₃ alkyl;
R⁸ represents H, C₁-C₆ alkyl, C₂-C₆ alkenyl, C₃-C₆ cycloalkyl, C₃-C₆ cycloalkyl(C₁-C₃ alkyl), 3-8 membered heterocyclyl, 3-8 membered heterocyclyl(C₁-C₆) alkyl, phenyl(C₁-C₆)alkyl or 5-6 membered heteroaryl(C₁-C₆)alkyl, wherein each C₁-C₆ alkyl, C₃-C₆ cycloalkyl, C₃-C₆ cycloalkyl(C₁-C₃ alkyl), phenyl, heteroaryl and 3-8 membered heterocyclyl is optionally independently substituted with 1-3 of halogen, halo(C₁-C₃ alkyl), hydroxy, oxo, C₁-C₃ alkyl, C₁-C₃ alkoxy, cyano, amino, mono- or di(C₁-C₃) alkyl amino, amino- or mono- or di(C₁-C₃) alkylaminocarbonyl, —SO₂R¹⁶, —SO₂(NR¹⁶)₂, or —NR¹⁷SO₂R¹⁶ wherein each R¹⁶ is independently C₁-C₆ alkyl and R¹⁷ is C₁-C₆ alkyl; or
R⁷ and R⁸ together with the nitrogen to which they are attached form ring Q, wherein ring Q is
   a 3-8 membered monocyclic heterocyclyl group, wherein the heterocyclyl group
      is optionally fused to an aromatic or non-aromatic ring containing 3-6 ring members of which 1 or 2 are optionally nitrogen, oxygen or sulfur atoms and the remainder carbons, or
      with a carbon atom from the monocyclic heterocyclyl group or fused ring system and additional carbon or hetero atoms, forms a spirocyclic ring system having up to 10 total ring members, wherein the ring members are carbons or 1 or 2 hetero atoms selected from nitrogen, sulfur and oxygen atoms, and
         wherein each ring Q is optionally substituted, on an aromatic or non-aromatic portion, with 1-4 R⁹ groups, wherein each R⁹ is independently (i) C₁-C₆ alkyl optionally substituted with C₁-C₃ alkoxy, (ii) C₁-C₆ alkoxy, (iii) C₃-C₆ cycloalkyl, (iv) cyano, (ii) hydroxy, (v) oxo, (vi) halogen, (vii) halo C₁-C₃ alkyl, (viii) hydroxy C₁-C₆ alkyl, (ix) amino, (x) mono- or di-(C₁-C₆ alkyl) amino where one alkyl is optionally substituted with oxo, (xi) C₁-C₆ alkanoyl optionally substituted with 1 or 2 of C₁-C₃ alkyl, C₁-C₃ alkoxy, C₃-C₆ cycloalkyl, cyano, hydroxy, oxo, halogen, halo C₁-C₃ alkyl, hydroxy C₁-C₃ alkyl, amino, mono- or di-(C₁-C₆ alkyl) amino, C₃-C₆ cycloalkyl, phenyl, 5-6 membered heteroaryl, wherein each C₁-C₃ alkyl and C₃-C₆ cycloalkyl on the alkanoyl is optionally substituted with halogen, hydroxy or amino, and wherein each cycloalkyl, phenyl and heteroaryl on the alkanoyl is optionally substituted with C₁-C₆ alkyl, C₁-C₆ alkoxy, halogen, amino, hydroxy, or cyano, (xii) C₃-C₆ cycloalkyloxy, (xiii) phenyl, (xiv) phenyl(C₁-C₃ alkanoyl), (xv) 5-7 membered heterocyclyl, or (xvi) 5-6 membered heteroaryl, wherein each phenyl, 5-7 membered heterocyclyl and 5-6 membered heteroaryl is optionally substituted with one to four C₁-C₃ alkyl, C₁-C₃ alkoxy, halogen, hydroxy, cyano, oxo, amino, mono- or di(C₁-C₃ alkyl)amino, amino(C₁-C₃) alkyl, mono- or di(C₁-C₃ alkyl)amino(C₁-C₃) alkyl, or amino- or mono- or di(C₁-C₃) alkylaminocarbonyl.

In another aspect, the present disclosure provides for a pharmaceutical composition comprising a compound or salt as otherwise described herein together with a pharmaceutically acceptable carrier, excipient or diluent.

In another aspect, the present disclosure provides for a method of treating a disease or disorder associated with modulation of phosphoinositide 3-kinase (PI3K), comprising administering to a patient in need thereof a therapeutically effective amount of a compound of any as otherwise described herein or a pharmaceutical composition as otherwise described herein.

In another aspect, the present disclosure provides for a method of inhibiting phosphoinositide 3-kinase (PI3K), comprising administering to a patient in need thereof a therapeutically effective amount of a compound as otherwise described herein or a pharmaceutical composition as otherwise described herein.

In another aspect, the present disclosure provides for a method of treating cancer or a disorder, the method comprising administering to a patient in need thereof a therapeutically effective amount of a compound as otherwise described herein or a pharmaceutical composition as otherwise described herein.

The compounds disclosed herein selectively bind to H1047R-mutated PI3Kα and not to wild-type PI3Kα.

In another aspect, the present disclosure provides intermediates as described herein, such intermediates being suitable for use in a process for preparing a compound as described herein.

Other aspects and embodiments of the disclosure are evident in view of the detailed description provided herein.

### DETAILED DESCRIPTION OF THE INVENTION

The present invention relates to inhibitors of PI3Kα. In particular, the present invention relates to compounds that inhibit PI3Kα activity, pharmaceutical compositions comprising a therapeutically effective amount of the compounds, and methods of use therefor.

### Definitions

Unless defined otherwise, all technical and scientific terms used herein have the same meaning as is commonly understood by one of skill in the art to which this invention belongs. All patents, patent applications, and publications referred to herein are incorporated by reference to the extent they are consistent with the present disclosure. Terms and ranges have their generally defined definition unless expressly defined otherwise.

For simplicity, chemical moieties are defined and referred to throughout primarily as univalent chemical moieties (e.g., alkyl, aryl, etc.). Nevertheless, such terms may also be used to convey corresponding multivalent moieties under the appropriate structural circumstances clear to those skilled in the art. For example, while an “alkyl” moiety generally refers to a monovalent radical (e.g. CH₃—CH₂—), in certain circumstances a bivalent linking moiety can be “alkyl,” in which case those skilled in the art will understand the alkyl to be a divalent radical (e.g., —CH₂—CH₂—), which is equivalent to the term “alkylene.” (Similarly, in circumstances in which a divalent moiety is required and is stated as being “aryl,” those skilled in the art will understand that the term “aryl” refers to the corresponding divalent moiety, arylene.) All atoms are understood to have their normal number of valences for bond formation (i.e., 4 for carbon, 3 for N, 2 for 0, and 2, 4, or 6 for S, depending on the oxidation state of the S).

The term “amino” refers to —NH₂.

The term “acetyl” refers to —C(O)CH₃.

As herein employed, the term “acyl” refers to an alkylcarbonyl or arylcarbonyl substituent wherein the alkyl and aryl portions are as defined herein.

The term “alkyl” as employed herein refers to saturated straight and branched chain aliphatic groups having from 1 to 12 carbon atoms. As such, “alkyl” encompasses C₁, C₂, C₃, C₄, C₅, C₆, C₇, C₈, C₉, C₁₀, C₁₁ and C₁₂ groups. Alkyl groups may be branched or unbranched. Examples of alkyl groups include, without limitation, methyl, ethyl, propyl, isopropyl, butyl, isobutyl, sec-butyl, tert-butyl, pentyl, and hexyl.

The term “alkenyl” as used herein means an unsaturated straight or branched chain aliphatic group with one or more carbon-carbon double bonds, having from 2 to 12 carbon atoms. As such, “alkenyl” encompasses C₂, C₃, C₄, C₅, C₆, C₇, C₈, C₉, C₁₀, C₁₁ and C₁₂ groups. Examples of alkenyl groups include, without limitation, ethenyl, propenyl, butenyl, pentenyl, and hexenyl.

The term “alkynyl” as used herein means an unsaturated straight or branched chain aliphatic group with one or more carbon-carbon triple bonds, having from 2 to 12 carbon atoms. As such, “alkynyl” encompasses C₂, C₃, C₄, C₅, C₆, C₇, C₈, C₉, C₁₀, C₁₁ and C₁₂ groups. Examples of alkynyl groups include, without limitation, ethynyl, propynyl, butynyl, pentynyl, and hexynyl.

An “alkylene,” “alkenylene,” or “alkynylene” group is an alkyl, alkenyl, or alkynyl group, as defined hereinabove, that is positioned between and serves to connect two other chemical groups. Examples of alkylene groups include, without limitation, methylene, ethylene, propylene, and butylene. Representative alkenylene groups include, without limitation, ethenylene, propenylene, and butenylene. Representative alkynylene groups include, without limitation, ethynylene, propynylene, and butynylene.

The term “alkoxy” refers to —O(C₁-C₆ alkyl).

The term “amino- or mono- or di(C₁-C₃) alkylaminocarbonyl” refers to carbamoyl groups, i.e., groups of the formula

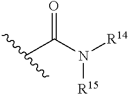

wherein R¹⁴ and R¹⁵ independently represent hydrogen and C₁-C₃ alkyl.

The term “cycloalkyl” as employed herein is a saturated and partially unsaturated cyclic hydrocarbon group having 3 to 12 carbons. As such, “cycloalkyl” includes C₃, C₄, C₅, C₆, C₇, C₈, C₉, C₁₀, C₁₁ and C₁₂ cyclic hydrocarbon groups. Examples of cycloalkyl groups include, without limitation, cyclopropyl, cyclobutyl, cyclopentyl, cyclopentenyl, cyclohexyl, cyclohexenyl, cycloheptyl, and cyclooctyl. The term “cycloalkyl” also encompasses bridged ring systems such as bicyclo[1.1.1]pentan-2-yl, bicyclo[1.1.1]pentan-1-yl. and bicyclo[2.2.2]octan-2-yl.

The term “C₃-C₆ cycloalkyloxy” refers to groups of the formula —O(C₃-C₆ cycloalkyl).

The term “heteroalkyl” refers to an alkyl group, as defined hereinabove, wherein one or more carbon atoms in the chain are independently replaced O, S, or NR^{x}, wherein R^{x} is hydrogen or C₁-C₃ alkyl. Examples of heteroalkyl groups include methoxymethyl, methoxyethyl and methoxypropyl.

An “aryl” group is a C₆-C₁₄ aromatic moiety comprising one to three aromatic rings. As such, “aryl” includes C₆, C₁₀, C₁₃, and C₁₄ cyclic hydrocarbon groups. A representative aryl group is a C₆-C₁₀ aryl group. Particular aryl groups include, without limitation, phenyl, naphthyl, anthracenyl, and fluorenyl. An “aryl” group also includes fused multicyclic (e.g., bicyclic) ring systems in which one or more of the fused rings is non-aromatic, provided that at least one ring is aromatic, such as indenyl.

An “aralkyl” or “arylalkyl” group comprises an aryl groucovalently linked to an alkyl group wherein the moiety is linked to another group via the alkyl moiety. An representative aralkyl group is —(C₁-C₆)alkyl(C₆-C₁₀)aryl, including, without limitation, benzyl, phenethyl, and naphthylmethyl. For example, an arC₁-C₃alkyl is an aryl group covalently linked to a C₁-C₃ alkyl.

As used herein, the term “fused” when used to define rings, e.g., bicyclic fused ring systems, refers to bicyclic, tricyclic, etc. ring systems sharing 2 or more atoms. Examples of such fused ring systems are (1S,4R)-2-azabicyclo[2.2.1]heptane; 2-azabicyclo[2.2.2]octane, 2,5-diazabicyclo[2.2.2]octane; 2-oxa-5-azabicyclo[2.2.2]octane; isoindoline; 1,2,3,4-tetrahydro-2,6-naphthyridine, 1,2,3,4-tetrahydroisoquinoline; 1,2,3,4-tetrahydro-1,4-(epiminomethano)naphthalene; and 3-azabicyclo[3.1.0]hexane.

A “heterocyclyl” or “heterocyclic” or “heterocycloalkyl” group is a mono- or bicyclic (fused or spiro) ring structure having from 3 to 12 atoms, (3, 4, 5, 6, 7, 8, 9, 10, 11 or 12 atoms), for example 4 to 8 atoms, wherein one or more ring atoms are independently —C(O)—, N, NR⁴, O, or S, and the remainder of the ring atoms are quaternary or carbonyl carbons. Examples of heterocyclic groups include, without limitation, epoxy, oxiranyl, oxetanyl, azetidinyl, aziridinyl, tetrahydrofuranyl, tetrahydropyranyl, tetrahydrothiophenyl, pyrrolidinyl, piperidinyl, piperazinyl, imidazolidinyl, thiazolidinyl, thiatanyl, dithianyl, trithianyl, azathianyl, oxathianyl, dioxolanyl, oxazolidinyl, oxazolidinonyl, decahydroquinolinyl, piperidonyl, 4-piperidonyl, thiomorpholinyl, dimethyl-morpholinyl, and morpholinyl. Examples of heterocyclic groups that are spiro ring systems are 6-azaspiro[2.5]octan-6-yl, 5-azaspiro[2.4]heptan-5-yl, 6-azaspiro[3.4]octan-6-yl, 5-oxa-7-azaspiro[3.4]octan-7-yl, 5,5-dimethyl-4-oxa-7-azaspiro[2.5]octan-7-yl, 2-oxa-6-azaspiro[3.3]heptan-6-yl, 1-oxa-6-azaspiro[3.4]octan-6-yl and 7,7-dimethyl-5-azaspiro[2.5]octan-5-yl. Specifically excluded from the scope of this term are compounds having adjacent ring O and/or S atoms. The heterocyclic groups can be attached to a parent group (i.e., the point of attachment) via any ring atom, including one of the heteroatoms or one of the carbon atoms, in the heterocyclic ring group. As chemically required, the heterocyclic ring may be attached to one or more other groups, for instance if operating as a bridging group. Thus, the term “heterocyclyl” includes groups such as azabicyclo[2.1.1]hexan-2-yl, azabicyclo[2.2.1]heptan-7-yl, 6-oxa-3-azabicyclo[3.1.1]heptan-3-yl, diazabicyclo[3.1.1]heptan-6-yl and hexahydro-3,5-methanocyclopenta[b]pyrrol-1(2H)-yl. The term “heterocyclyl” also includes fused multicyclic (e.g., bicyclic) ring systems in which one or more of the fused rings is aromatic or non-aromatic, provided that at least one ring is non-aromatic contains an N, O, or S ring atom. Examples of such fused multicyclic ring systems are indolinyl, indolin-2-yl, 2,3-dihydrobenzofuran-2-yl and 2,3,4,5-tetrahydrobenzo[d]oxazol-2-yl. Each of these examples is a 9-membered heterocyclyl.

As used herein, the term “heteroaryl” refers to a group having 5 to 14 ring atoms, preferably 5, 6, 10, 13 or 14 ring atoms; having 6, 10, or 14 π electrons shared in a cyclic array; and having, in addition to carbon atoms, from one to three heteroatoms that are each independently N, O, or S. “Heteroaryl” also includes fused multicyclic (e.g., bicyclic) ring systems in which one or more of the fused rings is non-aromatic, provided that at least one ring is aromatic and at least one ring contains an N, O, or S ring atom. The heteroaryl groups can be attached to a parent group (i.e., the point of attachment) via any ring atom, including one of the heteroatoms or one of the carbon atoms, in the heteroaryl ring group. As chemically required, the heteroaryl may be attached to one or more other groups, for instance if operating as a bridging group.

Examples of heteroaryl groups include acridinyl, azocinyl, benzimidazolyl, benzofuranyl, benzo[d]oxazol-2(3H)-one, 2H-benzo[b][1,4]oxazin-3(4H)-one, benzothiofuranyl, benzothiophenyl, benzoxazolyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, furanyl, furazanyl, imidazolinyl, imidazolyl, 1H-indazolyl, indolenyl, indolinyl, indolizinyl, indolyl, 3H-indolyl, isobenzofuranyl, isochromanyl, isoindazolyl, isoindolinyl, isoindolyl, isoquinolinyl, isothiazolyl, isoxazolyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinyl, pyrimidinyl, phenanthridinyl, phenanthrolinyl, phenazinyl, phenothiazinyl, phenoxathiinyl, phenoxazinyl, phthalazinyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolinyl, 2H-pyrrolyl, pyrrolyl, quinazolinyl, quinolinyl, 4H-quinolizinyl, quinoxalinyl, quinuclidinyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, and xanthenyl.

An “arylene,” “heteroarylene,” or “heterocyclylene” group is a bivalent aryl, heteroaryl, or heterocyclyl group, respectively, as defined hereinabove, that is positioned between and serves to connect two other chemical groups.

As employed herein, when a moiety (e.g., cycloalkyl, aryl, heteroaryl, heterocyclyl, urea, etc.) is described as “optionally substituted” without expressly stating the substituents it is meant that the group optionally has multiple non-hydrogen substituents, for example from one to five, or from one to four, or from one to three, or one or two, non-hydrogen substituents.

The term “halogen” or “halo” as employed herein refers to chlorine, bromine, fluorine, or iodine.

The term “haloalkyl” refers to an alkyl chain in which one or more hydrogens have been replaced by a halogen. Representative haloalkyls are trifluoromethyl, difluoromethyl, fluorochloromethyl, chloromethyl, and fluoromethyl.

The term “hydroxyalkyl” refers to -alkylene-OH.

It is to be understood that each individual atom present in Formula (I) and the compounds within formula (I), may be present in the form of any of its naturally occurring isotopes, with the most abundant isotope(s) being preferred. Thus, by way of example, each individual hydrogen atom present in formula (I), or in the formulae depicted hereinafter, may be present as a 1H, 2H (deuterium; D) or 3H (tritium; T) atom, preferably 1H. Similarly, by way of example, each individual carbon atom present in formula (I), or in the formulae depicted hereinafter, may be present as a 12C, 13C or 14C atom, preferably 12C.

Particular deuterated compounds of this disclosure are those wherein R^{5A} has one or more hydrogen atoms replaced with a deuterium. Representative deuterated R^{5A} groups are pyrrolidin-1-yl-d8, morpholino-d₈, (piperazin-1-yl-2,2,3,3,5,5,6,6-d8), and piperidine-1-yl-d10.

As used herein, “an effective amount” of a compound is an amount that is sufficient to negatively modulate or inhibit the activity of PI3Kα.

As used herein, a “therapeutically effective amount” of a compound is an amount that is sufficient to ameliorate or in some manner reduce a symptom or stop or reverse progression of a condition, or negatively modulate or inhibit the activity of PI3Kα. Such amount may be administered as a single dosage or may be administered according to a regimen, whereby it is effective.

As used herein, “treatment” means any manner in which the symptoms or pathology of a condition, disorder or disease in a patient are ameliorated or otherwise beneficially altered.

As used herein, “amelioration of the symptoms of a particular disorder by administration of a particular compound or pharmaceutical composition” refers to any lessening, whether permanent or temporary, lasting or transient, that can be attributed to or associated with administration of the composition.

### Compounds

In one aspect, the present disclosure provides compounds of Formula (I):

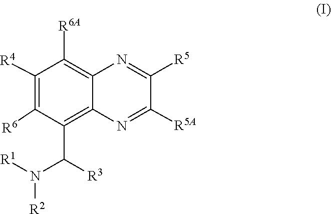

and pharmaceutically acceptable salts, prodrugs, solvates, hydrates, isomers, deuterated forms, and tautomers thereof, wherein:
R¹ is H, C₁-C₃ alkyl, or C₃-C₆ cycloalkyl;
R² is phenyl or a 5-6 membered heteroaryl group, wherein each phenyl and heteroaryl is optionally substituted with 1-5 R¹²;
   each R¹² is independently C₁-C₄ alkyl, —OR^{A}, —C(O)OR^{A}, (C₁-C₃ alkyl)-OR^{A}, —C(O)N(R^{A})₂, cyano, tetrazolyl or halogen;
      each R^{A} is independently H, C₁-C₆ alkyl, C₃-C₆ cycloalkyl, cycloalkyl or 2-carboxy-3,4,5-trihydroxytetrahydro-2H-pyran-6-yl;
R³ is C₁-C₃ alkyl optionally substituted, polysubstituted or persubstituted with fluoro, C₃-C₆ cycloalkyl, C₁-C₃ alkenyl, C₂-C₃ alkynyl, or C₅-C₆ cycloalkenyl;
R⁴ is H, halogen, C₁-C₃ alkyl, C₁-C₆ alkoxy, C₃-C₆ cycloalkyl, C₁-C₃ alkenyl, C₂-C₃ alkynyl, or C₅-C₆ cycloalkenyl or cyano, wherein each C₁-C₃ alkyl and C₃-C₆ cycloalkyl is optionally substituted with 1-5 halo groups;
R⁶ and R^{6A} independently represent H or C₁-C₆ alkyl;
R⁵ is hydrogen, —OR²⁰, —SR²⁰, C₁-C₆ alkyl, halogen, hydroxy, cyano, haloC₁-C₃ alkyl, hydroxy C₁-C₆ alkyl, C₁-C₆ alkoxy, C₁-C₆ alkoxy(C₁-C₆)alkyl, cyano, amino, mono- or di(C₁-C₃) alkyl amino, C₃-C₆ cycloalkyl, C₃-C₆ cycloalkyl(C₁-C₃ alkyl), 3-8 membered heterocyclyl, phenyl or 5-6 membered heteroaryl, wherein each alkyl, alkenyl, cycloalkyl, phenyl, 3-8 membered heterocyclyl and heteroaryl is optionally independently substituted with 1-3 of halogen, hydroxy, C₁-C₃ alkyl, C₁-C₃ alkoxy, haloC₁-C₃ alkyl, hydroxy C₁-C₆ alkyl, oxo, cyano, amino, mono-ordi(C₁-C₃) alkyl amino,
   wherein R²⁰ is aryl, aryl(C₁-C₆)alkyl, 5-7 membered heteroaryl or 5-7 membered heteroaryl(C₁-C₆)alkyl, wherein each aryl and heteroaryl is optionally independently substituted with one to four of C₁-C₆ alkyl, halogen, hydroxy, cyano, haloC₁-C₃ alkyl, hydroxy C₁-C₆ alkyl, C₁-C₆ alkoxy, C₁-C₆ alkoxy(C₁-C₆)alkyl, cyano, amino, or mono- or di(C₁-C₃) alkyl amino;
R^{5A} represents NR⁷R⁸, C₁-C₆ alkyl, C₃-C₆ cycloalkyl, C₃-C₆ cycloalkyl(C₁-C₃ alkyl), phenyl, or 5-6 membered heteroaryl, wherein each C₁-C₆ alkyl, C₃-C₆ cycloalkyl, C₃-C₆ cycloalkyl(C₁-C₃ alkyl), phenyl and heteroaryl is optionally independently substituted with 1-3 of halogen, halo(C₁-C₃ alkyl), hydroxy, oxo, C₁-C₃ alkyl, C₁-C₃ alkoxy, C₁-C₆ alkanoyl, cyano, amino, or mono- or di(C₁-C₃) alkyl amino;
R⁷ represents H or C₁-C₃ alkyl;
R⁸ represents H, C₁-C₆ alkyl, C₂-C₆ alkenyl, C₃-C₆ cycloalkyl, C₃-C₆ cycloalkyl(C₁-C₃ alkyl), 3-8 membered heterocyclyl, 3-8 membered heterocyclyl(C₁-C₆) alkyl, phenyl(C₁-C₆)alkyl or 5-6 membered heteroaryl(C₁-C₆)alkyl, wherein each C₁-C₆ alkyl, C₃-C₆ cycloalkyl, C₃-C₆ cycloalkyl(C₁-C₃ alkyl), phenyl, heteroaryl and 3-8 membered heterocyclyl is optionally independently substituted with 1-3 of halogen, halo(C₁-C₃ alkyl), hydroxy, oxo, C₁-C₃ alkyl, C₁-C₃ alkoxy, cyano, amino, mono- or di(C₁-C₃) alkyl amino, amino- or mono- or di(C₁-C₃) alkylaminocarbonyl, —SO₂R¹⁶, —SO₂(NR¹⁶)₂, or —NR¹⁷SO₂R¹⁶ wherein each R¹⁶ is independently C₁-C₆ alkyl and R¹⁷ is C₁-C₆ alkyl; or
R⁷ and R⁸ together with the nitrogen to which they are attached form ring Q, wherein ring Q is
   a 3-8 membered monocyclic heterocyclyl group, wherein the heterocyclyl group
      is optionally fused to an aromatic or non-aromatic ring containing 3-6 ring members of which 1 or 2 are optionally nitrogen, oxygen or sulfur atoms and the remainder carbons, or
      with a carbon atom from the monocyclic heterocyclyl group or fused ring system and additional carbon or hetero atoms, forms a spirocyclic ring system having up to 10 total ring members, wherein the ring members are carbons or 1 or 2 hetero atoms selected from nitrogen, sulfur and oxygen atoms, and
         wherein each ring Q is optionally substituted, on an aromatic or non-aromatic portion, with 1-4 R⁹ groups, wherein each R⁹ is independently (i) C₁-C₆ alkyl optionally substituted with C₁-C₃ alkoxy, (ii) C₁-C₆ alkoxy, (iii) C₃-C₆ cycloalkyl, (iv) cyano, (ii) hydroxy, (v) oxo, (vi) halogen, (vii) halo C₁-C₃ alkyl, (viii) hydroxy C₁-C₆ alkyl, (ix) amino, (x) mono- or di-(C₁-C₆ alkyl) amino where one alkyl is optionally substituted with oxo, (xi) C₁-C₆ alkanoyl optionally substituted with 1 or 2 of C₁-C₃ alkyl, C₁-C₃ alkoxy, C₃-C₆ cycloalkyl, cyano, hydroxy, oxo, halogen, halo C₁-C₃ alkyl, hydroxy C₁-C₃ alkyl, amino, mono- or di-(C₁-C₆ alkyl) amino, C₃-C₆ cycloalkyl, phenyl, 5-6 membered heteroaryl, wherein each C₁-C₃ alkyl and C₃-C₆ cycloalkyl on the alkanoyl is optionally substituted with halogen, hydroxy or amino, and wherein each cycloalkyl, phenyl and heteroaryl on the alkanoyl is optionally substituted with C₁-C₆ alkyl, C₁-C₆ alkoxy, halogen, amino, hydroxy, or cyano, (xii) C₃-C₆ cycloalkyloxy, (xiii) phenyl, (xiv) phenyl(C₁-C₃ alkanoyl), (xv) 5-7 membered heterocyclyl, or (xvi) 5-6 membered heteroaryl, wherein each phenyl, 5-7 membered heterocyclyl and 5-6 membered heteroaryl is optionally substituted with one to four C₁-C₃ alkyl, C₁-C₃ alkoxy, halogen, hydroxy, cyano, oxo, amino, mono- or di(C₁-C₃ alkyl)amino, amino(C₁-C₃) alkyl, mono- or di(C₁-C₃ alkyl)amino(C₁-C₃) alkyl, or amino- or mono- or di(C₁-C₃) alkylaminocarbonyl.

In another aspect, the present disclosure provides compounds of Formula (IV):

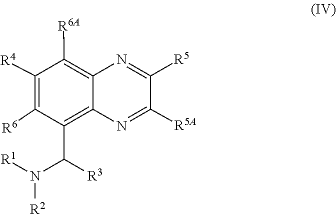

and pharmaceutically acceptable salts, prodrugs, solvates, hydrates, isomers, deuterated forms, and tautomers thereof, wherein:
R¹ is H, C₁-C₃ alkyl, or C₃-C₆ cycloalkyl;
R² is phenyl or a 5-6 membered heteroaryl group, wherein each phenyl and heteroaryl is optionally substituted with 1-5 R¹²;
   each R¹² is independently C₁-C₄ alkyl, —OR^{A}, —C(O)OR^{A}, (C₁-C₃ alkyl)-OR^{A}, —C(O)N(R^{A})₂, cyano, tetrazolyl, or halogen;
      each R^{A} is independently H, C₁-C₆ alkyl, or C₃-C₆ cycloalkyl; and
R³ is C₁-C₃ alkyl optionally substituted, polysubstituted or persubstituted with fluoro, C₃-C₆ cycloalkyl, C₁-C₃ alkenyl, C₂-C₃ alkynyl, or C₅-C₆ cycloalkenyl;
R⁴ is H, halogen, C₁-C₃ alkyl, C₃-C₆ cycloalkyl, C₁-C₃ alkenyl, C₂-C₃ alkynyl, or C₅-C₆ cycloalkenyl or cyano, wherein each C₁-C₃ alkyl and C₃-C₆ cycloalkyl is optionally substituted with 1-5 halo groups;
R⁶ and R^{6A} independently represent H or C₁-C₆ alkyl;
R⁵ is C₁-C₂ alkyl, cyclopropyl, bromo, chloro, fluoro, cyano, or trifluoromethyl;
R^{5A} represents NR⁷R⁸, C₁-C₆ alkyl, C₃-C₆ cycloalkyl, C₃-C₆ cycloalkyl(C₁-C₃ alkyl), phenyl or 5-6 membered heteroaryl, wherein each C₁-C₆ alkyl, C₃-C₆ cycloalkyl, C₃-C₆ cycloalkyl(C₁-C₃ alkyl), phenyl and heteroaryl is optionally substituted with 1-3 of halogen, halo(C₁-C₃ alkyl), hydroxy, C₁-C₃ alkyl, C₁-C₃ alkoxy, cyano, amino, or mono- or di(C₁-C₃) alkyl amino;
R⁷ represents H or C₁-C₃ alkyl;
R^{a} represent H, C₁-C₆ alkyl, C₃-C₆ cycloalkyl or C₃-C₆ cycloalkyl(C₁-C₃ alkyl) wherein each C₁-C₆ alkyl, C₃-C₆ cycloalkyl and C₃-C₆ cycloalkyl(C₁-C₃ alkyl) is optionally substituted with 1-3 of halogen, halo(C₁-C₃ alkyl), hydroxy, C₁-C₃ alkyl, C₁-C₃ alkoxy, cyano, amino, or mono- or di(C₁-C₃) alkyl amino; or
R⁷ and R⁸ together with the nitrogen to which they are attached form ring Q, wherein ring Q is
   a 3-8 membered monocyclic heterocyclyl group, wherein the heterocyclyl group
      is optionally fused to an aromatic or non-aromatic ring containing 3-6 ring members of which 1 or 2 are optionally nitrogen, oxygen or sulfur atoms and the remainder carbons, or
      with a carbon atom from the monocyclic heterocyclyl group and additional carbon or hetero atoms, forms a spirocyclic ring system having up to 10 total ring members, wherein the ring members are carbons or 1 or 2 hetero atoms selected from nitrogen, sulfur and oxygen atoms, and
         wherein each ring Q is optionally substituted, on an aromatic or non-aromatic portion, with 1-4 R⁹ groups, wherein each R⁹ is independently (i) C₁-C₆ alkyl, (ii) C₁-C₆ alkoxy, (iii) C₃-C₆ cycloalkyl, (iv) cyano, (ii) hydroxy, (v) oxo, (vi) halogen, (vii) halo C₁-C₃ alkyl, (viii) hydroxy C₁-C₆ alkyl, (ix) amino, (x) mono- or di-(C₁-C₆ alkyl) amino, (xi) C₂-C₆ alkanoyl optionally substituted with 1 or 2 of C₁-C₃ alkyl, C₁-C₃ alkoxy, C₃-C₆ cycloalkyl, cyano, hydroxy, oxo, halogen, halo C₁-C₃ alkyl, hydroxy C₁-C₃ alkyl, amino, or mono- or di-(C₁-C₆ alkyl) amino, wherein each C₁-C₃ alkyl or C₃-C₆ cycloalkyl on the alkanoyl is optionally substituted with halogen, hydroxy or amino, or (xii) C₃-C₆ cycloalkyloxy, or (xiii) phenyl, phenyl(C₁-C₃ alkanoyl), 5-7 membered heterocyclyl, or 5-6 membered heteroaryl where each phenyl, 5-7 membered heterocyclyl and 5-6 membered heteroaryl is optionally substituted with one to four C₁-C₃ alkyl, C₁-C₃ alkoxy, halogen, hydroxy, cyano, oxo, amino, or mono- or di(C₁-C₃ alkyl)amino.

In certain embodiments of formula (I) or formula (IV) as otherwise described herein R⁶ and R^{6A} represent hydrogen.

In certain embodiments of formula (I) or formula (IV) as otherwise described herein one of R⁶ and R^{6A} represents hydrogen and the other represents C₁-C₆ alkyl.

In certain embodiments of formula (I) or formula (IV) as otherwise described herein one of R⁶ and R^{6A} represents hydrogen and the other represents C₁-C₆ alkyl.

In certain embodiments of formula (I) or formula (IV) as otherwise described herein R⁶ and R^{6A} independently represent C₁-C₆ alkyl.

In certain embodiments of formula (I) or formula (IV) as otherwise described herein R¹ is H.

In certain embodiments of formula (I) or formula (IV) as otherwise described herein R¹ is CH₃.

In certain embodiments of formula (I) or formula (IV) as otherwise described herein R¹ is H or CH₃.

In certain embodiments of formula (I) or formula (IV) as otherwise described herein R² is phenyl or a 5-7 membered heteroaryl, each of which is substituted with 1, 2 or 3 R¹² groups.

In certain embodiments of formula (I) or formula (IV) as otherwise described herein R² is phenyl or a 5-7 membered heteroaryl, each of which is substituted with 1, 2 or 3 R¹² groups, and at least one R¹² group is —C(O)OR^{A}.

In certain embodiments of formula (I) or formula (IV) as otherwise described herein R² is phenyl or a 5-7 membered heteroaryl, each of which is substituted with 1, 2 or 3 R¹² groups, and at least one R¹² group is —C(O)OR^{A}, where R^{A} is H, methyl or ethyl.

In certain embodiments of formula (I) or formula (IV) as otherwise described herein R² is phenyl or a 5-7 membered heteroaryl, each of which is substituted with 1, 2 or 3 R¹² groups, and at least one R¹² group is —C(O)OR^{A}, where R^{A} is H.

In certain embodiments of formula (I) or formula (IV) as otherwise described herein R² is phenyl or a 5-7 membered heteroaryl, each of which is substituted with 1, 2 or 3 R¹² groups, and at least one R¹² group is —C(O)OR^{A}, where R^{A} is methyl or ethyl.

In certain embodiments of formula (I) or formula (IV) as otherwise described herein R² is phenyl or a 5-7 membered heteroaryl, each of which is substituted with 1, 2 or 3 R¹² groups, and at least one R¹² group is —C(O)OR^{A}, where R^{A} is 2-carboxy-3,4,5-trihydroxytetrahydro-2H-pyran-6-yl. A suitable compound to prepare compounds with such R^{A} groups is (2S,3S,4S,5R)-3,4,5,6-tetrahydroxytetrahydro-2H-pyran-2-carboxylic acid.

In certain embodiments of formula (I) or formula (IV) as otherwise described herein R² is phenyl or a 5-7 membered heteroaryl, each of which is substituted with 1, 2 or 3 R¹² groups, and at least one R¹² group is —C(O)OR^{A}.

In certain embodiments of formula (I) or formula (IV) as otherwise described herein R² is phenyl or a 5-7 membered heteroaryl, each of which is substituted with 1, 2 or 3 R¹² groups, and at least one R¹² group is a tetrazolyl, particularly 1H-tetrazol-5-yl.

In certain embodiments of formula (I) or formula (IV) as otherwise described herein R² is phenyl, pyridinyl, thienyl, thiazolyl, oxazolyl, isoxazolyl, isothiazolyl, imidazolyl, pyrazolyl, pyrazinyl, pyridazinyl or pyrimidinyl, each of which is optionally substituted with 1-5 R¹².

In certain embodiments of formula (I) or formula (IV) as otherwise described herein R² is phenyl or pyridinyl, each of which is optionally substituted with 1-5 R¹².

In certain embodiments of formula (I) or formula (IV) as otherwise described herein R² is phenyl or pyridinyl, each of which is optionally substituted with 1-5 R¹², and at least one R¹² group is —C(O)OR^{A}.

In certain embodiments of formula (I) or formula (IV) as otherwise described herein R² is phenyl or pyridinyl, each of which is optionally substituted with 1-5 R¹², and at least one R¹² group is —C(O)OR^{A}, where R^{A} is H, methyl or ethyl.

In certain embodiments of formula (I) or formula (IV) as otherwise described herein R² is phenyl or pyridinyl, each of which is optionally substituted with 1-5 R¹², and at least one R¹² group is —C(O)OR^{A}, where R^{A} is H.

In certain embodiments of formula (I) or formula (IV) as otherwise described herein R² is phenyl or pyridinyl, each of which is optionally substituted with 1-5 R¹², and at least one R¹² group is —C(O)OR^{A}, where R^{A} is methyl or ethyl.

In certain embodiments of formula (I) or formula (IV) as otherwise described herein R² is phenyl which is optionally substituted with 1-5 R¹².

In certain embodiments of formula (I) or formula (IV) as otherwise described herein R² is phenyl which is optionally substituted with 1-5 R¹², and at least one R¹² group is —C(O)OR^{A}.

In certain embodiments of formula (I) or formula (IV) as otherwise described herein R² is phenyl which is optionally substituted with 1-5 R¹², and at least one R¹² group is —C(O)OR^{A}, where R^{A} is H, methyl or ethyl.

In certain embodiments of formula (I) or formula (IV) as otherwise described herein R² is phenyl which is optionally substituted with 1-5 R¹², and at least one R¹² group is —C(O)OR^{A}, where R^{A} is H.

In certain embodiments of formula (I) or formula (IV) as otherwise described herein R² is phenyl which is optionally substituted with 1-5 R¹², and at least one R¹² group is —C(O)OR^{A}, where R^{A} is methyl or ethyl.

In certain embodiments of formula (I) or formula (IV) as otherwise described herein R² is pyridinyl which is optionally substituted with 1-5 R¹².

In certain embodiments of formula (I) or formula (IV) as otherwise described herein R² is pyridinyl which is optionally substituted with 1-5 R¹², and at least one R¹² group is —C(O)OR^{A}.

In certain embodiments of formula (I) or formula (IV) as otherwise described herein R² is pyridinyl which is optionally substituted with 1-5 R¹², and at least one R¹² group is —C(O)OR^{A}, where R^{A} is H, methyl or ethyl.

In certain embodiments of formula (I) or formula (IV) as otherwise described herein R² is pyridinyl which is optionally substituted with 1-5 R¹², and at least one R¹² group is —C(O)OR^{A}, where R^{A} is H.

In certain embodiments of formula (I) or formula (IV) as otherwise described herein R² is pyridinyl which is optionally substituted with 1-5 R¹², and at least one R¹² group is —C(O)OR^{A}, where R^{A} is methyl or ethyl.

In certain embodiments of formula (I) or formula (IV) as otherwise described herein R² is pyrimidinyl which is optionally substituted with 1-5 R¹².

In certain embodiments of formula (I) or formula (IV) as otherwise described herein R² is pyrimidinyl which is optionally substituted with 1-5 R¹², and at least one R¹² group is —C(O)OR^{A}.

In certain embodiments of formula (I) or formula (IV) as otherwise described herein R² is pyrimidinyl which is optionally substituted with 1-5 R¹², and at least one R¹² group is —C(O)OR^{A}, where R^{A} is H, methyl or ethyl.

In certain embodiments of formula (I) or formula (IV) as otherwise described herein R² is pyrimidinyl which is optionally substituted with 1-5 R¹², and at least one R¹² group is —C(O)OR^{A}, where R^{A} is H.

In certain embodiments of formula (I) or formula (IV) as otherwise described herein R² is pyrimidinyl which is optionally substituted with 1-5 R¹², and at least one R¹² group is —C(O)OR^{A}, where R^{A} is methyl or ethyl.

In certain embodiments of formula (I) or formula (IV) as otherwise described herein R³ is C₁-C₃ alkyl, wherein the alkyl group is unsubstituted, substituted with 1-5 halo groups, or perfluorinated.

In certain embodiments of formula (I) or formula (IV) as otherwise described herein R³ is C₁-C₃ alkyl, wherein the alkyl group is unsubstituted, substituted with 1-5 halo groups, or perfluorinated.

In certain embodiments of formula (I) or formula (IV) as otherwise described herein R³ is C₁-C₃ alkyl.

In certain embodiments of formula (I) or formula (IV) as otherwise described herein R³ is ethyl or R³ is methyl.

In certain embodiments of formula (I) as otherwise described herein R⁵ is hydrogen, C₁-C₃ alkyl, haloC₁-C₃ alkyl, halogen, cyano, C₃-C₆ cycloalkyl, or C₃-C₆ cycloalkyl(C₁-C₃ alkyl).

In certain embodiments of formula (I) as otherwise described herein R⁵ is haloC₁-C₃ alkyl, particularly, fluoromethyl, difluoromethyl or trifluoromethyl.

In certain embodiments of formula (I) as otherwise described herein R⁵ is phenyl or 5-6 membered heteroaryl, wherein each cycloalkyl, phenyl and heteroaryl is optionally substituted with 1-3 of halogen, hydroxy, C₁-C₃ alkyl, C₁-C₃ alkoxy, cyano, amino, or mono- or di(C₁-C₃) alkyl amino.

In certain embodiments of formula (I) as otherwise described herein R⁵ is hydrogen, C₁-C₃ alkyl, haloC₁-C₃ alkyl, halogen, cyano, cyclopropyl, or cyclopropyl(C₁-C₂ alkyl).

In certain embodiments of formula (I) as otherwise described herein R⁵ is methyl, ethyl, cyclopropyl or cyano.

In certain embodiments of formula (I) as otherwise described herein R⁵ is methyl or cyano.

In certain embodiments of formula (I) as otherwise described herein R⁵ is C₁-C₃ alkyl, haloC₁-C₃ alkyl, halogen, cyano, cyclopropyl, or cyclopropyl(C₁-C₂ alkyl).

In certain embodiments of formula (I) as otherwise described herein R⁵ is hydrogen, halogen, hydroxy, cyano or amino.

In certain embodiments of formula (I) as otherwise described herein R⁵ is cycloalkyl, phenyl or 5-6 membered heteroaryl, where each cycloalkyl, phenyl and heteroaryl is optionally substituted with 1-3 of halogen, hydroxy, C₁-C₂ alkyl, C₁-C₂ alkoxy, cyano, amino, or mono- or di(C₁-C₂) alkylamino.

In certain embodiments of formula (I) as otherwise described herein R⁵ is phenyl or 5-6 membered heteroaryl, where each cycloalkyl, phenyl and heteroaryl is optionally substituted with 1 or 2 of halogen, hydroxy, C₁-C₂ alkyl, C₁-C₂ alkoxy, cyano, amino, or mono- or di(C₁-C₂) alkylamino.

In certain embodiments of formula (I) as otherwise described herein R⁵ is heterocyclyl connected to the parent ring system at a carbon atom and which heterocyclyl is optionally substituted with 1-3 of halogen, hydroxy, C₁-C₂ alkyl, C₁-C₂ alkoxy, cyano, amino, or mono- or di(C₁-C₂) alkylamino.

In certain embodiments of formula (IV) as otherwise described herein R⁵ is methyl.

In certain embodiments of formula (IV) as otherwise described herein R⁵ is ethyl.

In certain embodiments of formula (IV) as otherwise described herein R⁵ is cyano.

In certain embodiments of formula (IV) as otherwise described herein R⁵ is cyclopropyl.

In certain embodiments of formula (IV) as otherwise described herein R⁵ is bromo.

In certain embodiments of formula (IV) as otherwise described herein R⁵ is fluoro.

In certain embodiments of formula (IV) as otherwise described herein R⁵ is chloro.

In certain embodiments of formula (IV) as otherwise described herein R⁵ is trifluoromethyl.

In certain embodiments of formula (I) or formula (IV) as otherwise described herein R² is optionally substituted phenyl.

In certain embodiments of formula (I) or formula (IV) as otherwise described herein R² is optionally substituted pyridinyl.

In certain embodiments of formula (I) or formula (IV) as otherwise described herein R⁴ is hydrogen, fluoro, chloro, methyl, ethyl, trifluoromethyl or cyano.

In certain embodiments of formula (I) or formula (IV) as otherwise described herein R⁴ is hydrogen, fluoro, chloro, methyl, trifluoromethyl or cyano.

In certain embodiments of formula (I) or formula (IV) as otherwise described herein R⁴ is fluoro, chloro, methyl, cyclopropyl or cyano.

In certain embodiments of formula (I) or formula (IV) as otherwise described herein R⁴ is fluoro, chloro, methyl, cyclopropyl, trifluoromethyl or cyano.

In certain embodiments of formula (I) or formula (IV) as otherwise described herein R⁴ is fluoro, methyl, trifluoromethyl or cyano.

In certain embodiments of formula (I) or formula (IV) as otherwise described herein R⁴ is fluoro, methyl or cyano.

In certain embodiments of formula (I) or formula (IV) as otherwise described herein R^{5A} represents C₁-C₆ alkyl, C₃-C₆ cycloalkyl or C₃-C₆ cycloalkyl(C₁-C₃ alkyl);

In certain embodiments of formula (I) or formula (IV) as otherwise described herein R^{5A} represents NR⁷R⁸.

In certain embodiments of formula (I) or formula (IV) as otherwise described herein R⁷ is hydrogen and R⁸ represents hydrogen, C₁-C₆ alkyl, C₃-C₆ cycloalkyl or C₃-C₆ cycloalkyl(C₁-C₃ alkyl).

In certain embodiments of formula (I) or formula (IV) as otherwise described herein R⁷ and R⁸ together with the nitrogen to which they are attached form ring Q, wherein ring Q is
a 3-7 membered monocyclic heterocyclyl group, and
   the heterocyclyl group is optionally substituted with 1-4 R⁹ groups, wherein each R⁹ is independently C₁-C₆ alkyl, C₁-C₆ alkoxy, C₃-C₆ cycloalkyl, cyano, hydroxy, oxo, halogen, halo C₁-C₃ alkyl, hydroxy C₁-C₆ alkyl, amino, mono- or di-(C₁-C₆ alkyl) amino, or phenyl or phenyl(C₁-C₃ alkanoyl) where each phenyl is optionally substituted with C₁-C₃ alkyl, C₁-C₃ alkoxy, halogen, hydroxy, cyano, amino, or mono- or di(C₁-C₃ alkyl)amino.

In certain embodiments of formula (I) or formula (IV) as otherwise described herein R⁷ and R⁸ together with the nitrogen to which they are attached form ring Q, wherein ring Q is
a 3-7 membered monocyclic heterocyclyl group, and
   the heterocyclyl group is fused to an aromatic or non-aromatic ring containing 3-6 ring members of which 1 or 2 are optionally nitrogen atoms and the remainder carbons, wherein
      the fused heterocyclyl group is optionally substituted, on an aromatic or non-aromatic portion, with 1-4 R⁹ groups, wherein each R⁹ is independently C₁-C₆ alkyl, C₁-C₆ alkoxy, C₃-C₆ cycloalkyl, cyano, hydroxy, oxo, halogen, halo C₁-C₃ alkyl, hydroxy C₁-C₆ alkyl, amino, mono- or di-(C₁-C₆ alkyl) amino, or phenyl or phenyl(C₁-C₃ alkanoyl) where each phenyl is optionally substituted with C₁-C₃ alkyl, C₁-C₃ alkoxy, halogen, hydroxy, cyano, amino, or mono- or di(C₁-C₃ alkyl)amino.

In certain embodiments of formula (I) or formula (IV) as otherwise described herein R⁷ and R⁸ together with the nitrogen to which they are attached form a 3-7 membered monocyclic heterocyclyl group, and
the heterocyclyl group forms a spirocyclic ring system having up to 10 total ring members with a carbon atom from the monocyclic heterocyclyl group and additional carbon, nitrogen, sulfur or oxygen atoms, wherein
   wherein the spirocyclic ring system is optionally substituted with 1-4 R⁹ groups, wherein each R⁹ is independently C₁-C₆ alkyl, C₁-C₆ alkoxy, C₃-C₆ cycloalkyl, cyano, hydroxy, oxo, halogen, halo C₁-C₃ alkyl, hydroxy C₁-C₆ alkyl, amino, mono- or di-(C₁-C₆ alkyl) amino, or phenyl or phenyl(C₁-C₃ alkanoyl) where each phenyl is optionally substituted with C₁-C₃ alkyl, C₁-C₃ alkoxy, halogen, hydroxy, cyano, amino, or mono- or di(C₁-C₃ alkyl)amino.

In certain embodiments of formula (I) or formula (IV) as otherwise described herein R⁷ and R⁸ together with the nitrogen to which they are attached form a piperidinyl, piperazinyl or morpholinyl group, each of which is optionally substituted with 1-4 R⁹ groups, wherein each R⁹ is independently C₁-C₆ alkyl, C₁-C₆ alkoxy, C₃-C₆ cycloalkyl, cyano, hydroxy, oxo, halogen, halo C₁-C₃ alkyl, hydroxy C₁-C₆ alkyl, amino, mono- or di-(C₁-C₆ alkyl) amino, or phenyl or phenyl(C₁-C₃ alkanoyl) where each phenyl is optionally substituted with C₁-C₃ alkyl, C₁-C₃ alkoxy, halogen, hydroxy, cyano, amino, or mono- or di(C₁-C₃ alkyl)amino.

In certain embodiments of formula (I) or formula (IV) as otherwise described herein R⁷ and R⁸ together with the nitrogen to which they are attached form a piperidinyl, piperazinyl or morpholinyl group, each of which is optionally substituted with 1-4 R⁹ groups, wherein each R⁹ is independently C₁-C₃ alkyl, C₁-C₃ alkoxy, C₃-C₆ cycloalkyl, cyano, hydroxy, oxo, halogen, halo C₁-C₃ alkyl, hydroxy C₁-C₃ alkyl, amino, or mono- or di-(C₁-C₃ alkyl) amino.

In certain embodiments of formula (I) or formula (IV) as otherwise described herein R⁷ and R⁸ together with the nitrogen to which they are attached form a piperidinyl, piperazinyl or morpholinyl group, each of which is optionally substituted with 1-4 R⁹ groups, wherein each R⁹ is independently methyl, ethyl, methoxy, ethoxy, cyano, fluoro, chloro, trifluoromethyl, cyclopropyl, or cyclopropylmethyl.

In another aspect, the present disclosure provides compounds of Formula (IV-A):

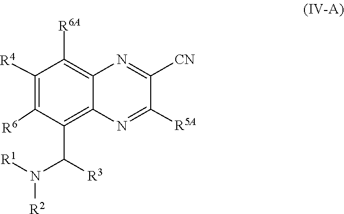

and pharmaceutically acceptable salts, prodrugs, solvates, hydrates, isomers, deuterated forms, and tautomers thereof, wherein:
R¹ is H, C₁-C₃ alkyl, or C₃-C₆ cycloalkyl;
R² is phenyl or a 5-6 membered heteroaryl group, wherein each phenyl and heteroaryl is optionally substituted with 1-5 R¹²;
   each R¹² is independently C₁-C₄ alkyl, —OR^{A}, —C(O)OR^{A}, (C₁-C₃ alkyl)-OR^{A}, —C(O)N(R^{A})₂, cyano, or halogen;
      each R^{A} is independently H, C₁-C₆ alkyl, or C₃-C₆ cycloalkyl; and
R³ is C₁-C₃ alkyl optionally substituted, polysubstituted or persubstituted with fluoro, C₃-C₆ cycloalkyl, C₁-C₃ alkenyl, C₂-C₃ alkynyl, or C₅-C₆ cycloalkenyl;
R⁴ is H, halogen, C₁-C₃ alkyl, C₃-C₆ cycloalkyl, C₁-C₃ alkenyl, C₂-C₃ alkynyl, or C₅-C₆ cycloalkenyl or cyano, wherein each C₁-C₃ alkyl and C₃-C₆ cycloalkyl is optionally substituted with 1-5 halo groups;
R⁶ and R^{6A} independently represent H or C₁-C₆ alkyl;
R^{5A} represents NR⁷R⁸, C₁-C₆ alkyl, C₃-C₆ cycloalkyl, C₃-C₆ cycloalkyl(C₁-C₃ alkyl), phenyl or 5-6 membered heteroaryl, wherein each C₁-C₆ alkyl, C₃-C₆ cycloalkyl, C₃-C₆ cycloalkyl(C₁-C₃ alkyl), phenyl and heteroaryl is optionally substituted with 1-3 of halogen, halo(C₁-C₃ alkyl), hydroxy, C₁-C₃ alkyl, C₁-C₃ alkoxy, cyano, amino, or mono- or di(C₁-C₃) alkyl amino;
R⁷ represents H or C₁-C₃ alkyl;
R⁸ represent H, C₁-C₆ alkyl, C₃-C₆ cycloalkyl or C₃-C₆ cycloalkyl(C₁-C₃ alkyl) wherein each C₁-C₆ alkyl, C₃-C₆ cycloalkyl and C₃-C₆ cycloalkyl(C₁-C₃ alkyl) is optionally substituted with 1-3 of halogen, halo(C₁-C₃ alkyl), hydroxy, C₁-C₃ alkyl, C₁-C₃ alkoxy, cyano, amino, or mono- or di(C₁-C₃) alkyl amino; or
R⁷ and R⁸ together with the nitrogen to which they are attached form ring Q, wherein ring Q is
   a 3-8 membered monocyclic heterocyclyl group, wherein the heterocyclyl group
      is optionally fused to an aromatic or non-aromatic ring containing 3-6 ring members of which 1 or 2 are optionally nitrogen, oxygen or sulfur atoms and the remainder carbons, or
      with a carbon atom from the monocyclic heterocyclyl group and additional carbon or hetero atoms, forms a spirocyclic ring system having up to 10 total ring members, wherein the ring members are carbons or 1 or 2 hetero atoms selected from nitrogen, sulfur and oxygen atoms, and
         wherein each ring Q is optionally substituted, on an aromatic or non-aromatic portion, with 1-4 R⁹ groups, wherein each R⁹ is independently (i) C₁-C₆ alkyl, (ii) C₁-C₆ alkoxy, (iii) C₃-C₆ cycloalkyl, (iv) cyano, (ii) hydroxy, (v) oxo, (vi) halogen, (vii) halo C₁-C₃ alkyl, (viii) hydroxy C₁-C₆ alkyl, (ix) amino, (x) mono- or di-(C₁-C₆ alkyl) amino, (xi) C₂-C₆ alkanoyl optionally substituted with 1 or 2 of C₁-C₃ alkyl, C₁-C₃ alkoxy, C₃-C₆ cycloalkyl, cyano, hydroxy, oxo, halogen, halo C₁-C₃ alkyl, hydroxy C₁-C₃ alkyl, amino, or mono- or di-(C₁-C₆ alkyl) amino, wherein each C₁-C₃ alkyl or C₃-C₆ cycloalkyl on the alkanoyl is optionally substituted with halogen, hydroxy or amino, or (xii) C₃-C₆ cycloalkyloxy, or (xiii) phenyl, phenyl(C₁-C₃ alkanoyl), 5-7 membered heterocyclyl, or 5-6 membered heteroaryl where each phenyl, 5-7 membered heterocyclyl and 5-6 membered heteroaryl is optionally substituted with one to four C₁-C₃ alkyl, C₁-C₃ alkoxy, halogen, hydroxy, cyano, oxo, amino, or mono- or di(C₁-C₃ alkyl)amino.

In another aspect, the present disclosure provides compounds of Formula (IV-B):

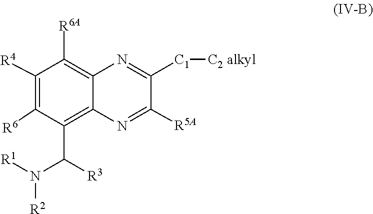

and pharmaceutically acceptable salts, prodrugs, solvates, hydrates, isomers, deuterated forms, and tautomers thereof, wherein:
R¹ is H, C₁-C₃ alkyl, or C₃-C₆ cycloalkyl;
R² is phenyl or a 5-6 membered heteroaryl group, wherein each phenyl and heteroaryl is optionally substituted with 1-5 R¹²;
   each R¹² is independently C₁-C₄ alkyl, —OR^{A}, —C(O)OR^{A}, (C₁-C₃ alkyl)-OR^{A}, —C(O)N(R^{A})₂, cyano, or halogen;
      each R^{A} is independently H, C₁-C₆ alkyl, or C₃-C₆ cycloalkyl; and
R³ is C₁-C₃ alkyl optionally substituted, polysubstituted or persubstituted with fluoro, C₃-C₆ cycloalkyl, C₁-C₃ alkenyl, C₂-C₃ alkynyl, or C₅-C₆ cycloalkenyl;
R⁴ is H, halogen, C₁-C₃ alkyl, C₃-C₆ cycloalkyl, C₁-C₃ alkenyl, C₂-C₃ alkynyl, or C₅-C₆ cycloalkenyl or cyano, wherein each C₁-C₃ alkyl and C₃-C₆ cycloalkyl is optionally substituted with 1-5 halo groups;
R⁶ and R^{6A} independently represent H or C₁-C₆ alkyl;
R^{5A} represents NR⁷R⁸, C₁-C₆ alkyl, C₃-C₆ cycloalkyl, C₃-C₆ cycloalkyl(C₁-C₃ alkyl), phenyl or 5-6 membered heteroaryl, wherein each C₁-C₆ alkyl, C₃-C₆ cycloalkyl, C₃-C₆ cycloalkyl(C₁-C₃ alkyl), phenyl and heteroaryl is optionally substituted with 1-3 of halogen, halo(C₁-C₃ alkyl), hydroxy, C₁-C₃ alkyl, C₁-C₃ alkoxy, cyano, amino, or mono- or di(C₁-C₃) alkyl amino;
R⁷ represents H or C₁-C₃ alkyl;
R⁸ represent H, C₁-C₆ alkyl, C₃-C₆ cycloalkyl or C₃-C₆ cycloalkyl(C₁-C₃ alkyl) wherein each C₁-C₆ alkyl, C₃-C₆ cycloalkyl and C₃-C₆ cycloalkyl(C₁-C₃ alkyl) is optionally substituted with 1-3 of halogen, halo(C₁-C₃ alkyl), hydroxy, C₁-C₃ alkyl, C₁-C₃ alkoxy, cyano, amino, or mono- or di(C₁-C₃) alkyl amino; or
R⁷ and R⁸ together with the nitrogen to which they are attached form ring Q, wherein ring Q is
   a 3-8 membered monocyclic heterocyclyl group, wherein the heterocyclyl group
      is optionally fused to an aromatic or non-aromatic ring containing 3-6 ring members of which 1 or 2 are optionally nitrogen, oxygen or sulfur atoms and the remainder carbons, or
      with a carbon atom from the monocyclic heterocyclyl group and additional carbon or hetero atoms, forms a spirocyclic ring system having up to 10 total ring members, wherein the ring members are carbons or 1 or 2 hetero atoms selected from nitrogen, sulfur and oxygen atoms, and
         wherein each ring Q is optionally substituted, on an aromatic or non-aromatic portion, with 1-4 R⁹ groups, wherein each R⁹ is independently (i) C₁-C₆ alkyl, (ii) C₁-C₆ alkoxy, (iii) C₃-C₆ cycloalkyl, (iv) cyano, (ii) hydroxy, (v) oxo, (vi) halogen, (vii) halo C₁-C₃ alkyl, (viii) hydroxy C₁-C₆ alkyl, (ix) amino, (x) mono- or di-(C₁-C₆ alkyl) amino, (xi) C₂-C₆ alkanoyl optionally substituted with 1 or 2 of C₁-C₃ alkyl, C₁-C₃ alkoxy, C₃-C₆ cycloalkyl, cyano, hydroxy, oxo, halogen, halo C₁-C₃ alkyl, hydroxy C₁-C₃ alkyl, amino, or mono- or di-(C₁-C₆ alkyl) amino, wherein each C₁-C₃ alkyl or C₃-C₆ cycloalkyl on the alkanoyl is optionally substituted with halogen, hydroxy or amino, or (xii) C₃-C₆ cycloalkyloxy, or (xiii) phenyl, phenyl(C₁-C₃ alkanoyl), 5-7 membered heterocyclyl, or 5-6 membered heteroaryl where each phenyl, 5-7 membered heterocyclyl and 5-6 membered heteroaryl is optionally substituted with one to four C₁-C₃ alkyl, C₁-C₃ alkoxy, halogen, hydroxy, cyano, oxo, amino, or mono- or di(C₁-C₃ alkyl)amino.

In another aspect, the present disclosure provides compounds of Formula (IV-C):

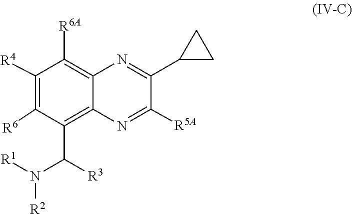

and pharmaceutically acceptable salts, prodrugs, solvates, hydrates, isomers, deuterated forms, and tautomers thereof, wherein:
R¹ is H, C₁-C₃ alkyl, or C₃-C₆ cycloalkyl;
R² is phenyl or a 5-6 membered heteroaryl group, wherein each phenyl and heteroaryl is optionally substituted with 1-5 R¹²;
   each R¹² is independently C₁-C₄ alkyl, —OR^{A}, —C(O)OR^{A}, (C₁-C₃ alkyl)-OR^{A}, —C(O)N(R^{A})₂, cyano, or halogen;
      each R^{A} is independently H, C₁-C₆ alkyl, or C₃-C₆ cycloalkyl; and
R³ is C₁-C₃ alkyl optionally substituted, polysubstituted or persubstituted with fluoro, C₃-C₆ cycloalkyl, C₁-C₃ alkenyl, C₂-C₃ alkynyl, or C₅-C₆ cycloalkenyl;
R⁴ is H, halogen, C₁-C₃ alkyl, C₃-C₆ cycloalkyl, C₁-C₃ alkenyl, C₂-C₃ alkynyl, or C₅-C₆ cycloalkenyl or cyano, wherein each C₁-C₃ alkyl and C₃-C₆ cycloalkyl is optionally substituted with 1-5 halo groups;
R⁶ and R^{6A} independently represent H or C₁-C₆ alkyl;
R^{5A} represents NR⁷R⁸, C₁-C₆ alkyl, C₃-C₆ cycloalkyl, C₃-C₆ cycloalkyl(C₁-C₃ alkyl), phenyl or 5-6 membered heteroaryl, wherein each C₁-C₆ alkyl, C₃-C₆ cycloalkyl, C₃-C₆ cycloalkyl(C₁-C₃ alkyl), phenyl and heteroaryl is optionally substituted with 1-3 of halogen, halo(C₁-C₃ alkyl), hydroxy, C₁-C₃ alkyl, C₁-C₃ alkoxy, cyano, amino, or mono- or di(C₁-C₃) alkyl amino;
R⁷ represents H or C₁-C₃ alkyl;
R⁸ represent H, C₁-C₆ alkyl, C₃-C₆ cycloalkyl or C₃-C₆ cycloalkyl(C₁-C₃ alkyl) wherein each C₁-C₆ alkyl, C₃-C₆ cycloalkyl and C₃-C₆ cycloalkyl(C₁-C₃ alkyl) is optionally substituted with 1-3 of halogen, halo(C₁-C₃ alkyl), hydroxy, C₁-C₃ alkyl, C₁-C₃ alkoxy, cyano, amino, or mono- or di(C₁-C₃) alkyl amino; or
R⁷ and R⁸ together with the nitrogen to which they are attached form ring Q, wherein ring Q is
   a 3-8 membered monocyclic heterocyclyl group, wherein the heterocyclyl group
      is optionally fused to an aromatic or non-aromatic ring containing 3-6 ring members of which 1 or 2 are optionally nitrogen, oxygen or sulfur atoms and the remainder carbons, or
      with a carbon atom from the monocyclic heterocyclyl group and additional carbon or hetero atoms, forms a spirocyclic ring system having up to 10 total ring members, wherein the ring members are carbons or 1 or 2 hetero atoms selected from nitrogen, sulfur and oxygen atoms, and
         wherein each ring Q is optionally substituted, on an aromatic or non-aromatic portion, with 1-4 R⁹ groups, wherein each R⁹ is independently (i) C₁-C₆ alkyl, (ii) C₁-C₆ alkoxy, (iii) C₃-C₆ cycloalkyl, (iv) cyano, (ii) hydroxy, (v) oxo, (vi) halogen, (vii) halo C₁-C₃ alkyl, (viii) hydroxy C₁-C₆ alkyl, (ix) amino, (x) mono- or di-(C₁-C₆ alkyl) amino, (xi) C₂-C₆ alkanoyl optionally substituted with 1 or 2 of C₁-C₃ alkyl, C₁-C₃ alkoxy, C₃-C₆ cycloalkyl, cyano, hydroxy, oxo, halogen, halo C₁-C₃ alkyl, hydroxy C₁-C₃ alkyl, amino, or mono- or di-(C₁-C₆ alkyl) amino, wherein each C₁-C₃ alkyl or C₃-C₆ cycloalkyl on the alkanoyl is optionally substituted with halogen, hydroxy or amino, or (xii) C₃-C₆ cycloalkyloxy, or (xiii) phenyl, phenyl(C₁-C₃ alkanoyl), 5-7 membered heterocyclyl, or 5-6 membered heteroaryl where each phenyl, 5-7 membered heterocyclyl and 5-6 membered heteroaryl is optionally substituted with one to four C₁-C₃ alkyl, C₁-C₃ alkoxy, halogen, hydroxy, cyano, oxo, amino, or mono- or di(C₁-C₃ alkyl)amino.

In another aspect, the present disclosure provides compounds of Formula (IV-D):

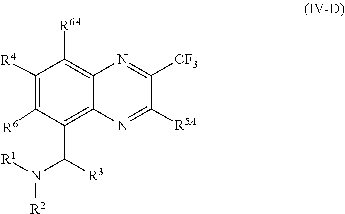

and pharmaceutically acceptable salts, prodrugs, solvates, hydrates, isomers, deuterated forms, and tautomers thereof, wherein:
R¹ is H, C₁-C₃ alkyl, or C₃-C₆ cycloalkyl;
R² is phenyl or a 5-6 membered heteroaryl group, wherein each phenyl and heteroaryl is optionally substituted with 1-5 R¹²;
   each R¹² is independently C₁-C₄ alkyl, —OR^{A}, —C(O)OR^{A}, (C₁-C₃ alkyl)-OR^{A}, —C(O)N(R^{A})₂, cyano, or halogen;
      each R^{A} is independently H, C₁-C₆ alkyl, or C₃-C₆ cycloalkyl; and
R³ is C₁-C₃ alkyl optionally substituted, polysubstituted or persubstituted with fluoro, C₃-C₆ cycloalkyl, C₁-C₃ alkenyl, C₂-C₃ alkynyl, or C₅-C₆ cycloalkenyl;
R⁴ is H, halogen, C₁-C₃ alkyl, C₃-C₆ cycloalkyl, C₁-C₃ alkenyl, C₂-C₃ alkynyl, or C₅-C₆ cycloalkenyl or cyano, wherein each C₁-C₃ alkyl and C₃-C₆ cycloalkyl is optionally substituted with 1-5 halo groups;
R⁶ and R^{6A} independently represent H or C₁-C₆ alkyl;
R^{5A} represents NR⁷R⁸, C₁-C₆ alkyl, C₃-C₆ cycloalkyl, C₃-C₆ cycloalkyl(C₁-C₃ alkyl), phenyl or 5-6 membered heteroaryl, wherein each C₁-C₆ alkyl, C₃-C₆ cycloalkyl, C₃-C₆ cycloalkyl(C₁-C₃ alkyl), phenyl and heteroaryl is optionally substituted with 1-3 of halogen, halo(C₁-C₃ alkyl), hydroxy, C₁-C₃ alkyl, C₁-C₃ alkoxy, cyano, amino, or mono- or di(C₁-C₃) alkyl amino;
R⁷ represents H or C₁-C₃ alkyl;
R⁸ represent H, C₁-C₆ alkyl, C₃-C₆ cycloalkyl or C₃-C₆ cycloalkyl(C₁-C₃ alkyl) wherein each C₁-C₆ alkyl, C₃-C₆ cycloalkyl and C₃-C₆ cycloalkyl(C₁-C₃ alkyl) is optionally substituted with 1-3 of halogen, halo(C₁-C₃ alkyl), hydroxy, C₁-C₃ alkyl, C₁-C₃ alkoxy, cyano, amino, or mono- or di(C₁-C₃) alkyl amino; or
R⁷ and R⁸ together with the nitrogen to which they are attached form ring Q, wherein ring Q is
   a 3-8 membered monocyclic heterocyclyl group, wherein the heterocyclyl group
      is optionally fused to an aromatic or non-aromatic ring containing 3-6 ring members of which 1 or 2 are optionally nitrogen, oxygen or sulfur atoms and the remainder carbons, or
      with a carbon atom from the monocyclic heterocyclyl group and additional carbon or hetero atoms, forms a spirocyclic ring system having up to 10 total ring members, wherein the ring members are carbons or 1 or 2 hetero atoms selected from nitrogen, sulfur and oxygen atoms, and
         wherein each ring Q is optionally substituted, on an aromatic or non-aromatic portion, with 1-4 R⁹ groups, wherein each R⁹ is independently (i) C₁-C₆ alkyl, (ii) C₁-C₆ alkoxy, (iii) C₃-C₆ cycloalkyl, (iv) cyano, (ii) hydroxy, (v) oxo, (vi) halogen, (vii) halo C₁-C₃ alkyl, (viii) hydroxy C₁-C₆ alkyl, (ix) amino, (x) mono- or di-(C₁-C₆ alkyl) amino, (xi) C₂-C₆ alkanoyl optionally substituted with 1 or 2 of C₁-C₃ alkyl, C₁-C₃ alkoxy, C₃-C₆ cycloalkyl, cyano, hydroxy, oxo, halogen, halo C₁-C₃ alkyl, hydroxy C₁-C₃ alkyl, amino, or mono- or di-(C₁-C₆ alkyl) amino, wherein each C₁-C₃ alkyl or C₃-C₆ cycloalkyl on the alkanoyl is optionally substituted with halogen, hydroxy or amino, or (xii) C₃-C₆ cycloalkyloxy, or (xiii) phenyl, phenyl(C₁-C₃ alkanoyl), 5-7 membered heterocyclyl, or 5-6 membered heteroaryl where each phenyl, 5-7 membered heterocyclyl and 5-6 membered heteroaryl is optionally substituted with one to four C₁-C₃ alkyl, C₁-C₃ alkoxy, halogen, hydroxy, cyano, oxo, amino, or mono- or di(C₁-C₃ alkyl)amino.

In another aspect, the present disclosure provides compounds of Formula (IV-E):

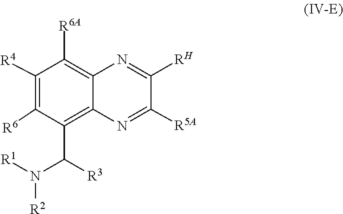

and pharmaceutically acceptable salts, prodrugs, solvates, hydrates, isomers, deuterated forms, and tautomers thereof, wherein:
R¹ is H, C₁-C₃ alkyl, or C₃-C₆ cycloalkyl;
R² is phenyl or a 5-6 membered heteroaryl group, wherein each phenyl and heteroaryl is optionally substituted with 1-5 R¹²;
   each R¹² is independently C₁-C₄ alkyl, —OR^{A}, —C(O)OR^{A}, (C₁-C₃ alkyl)-OR^{A}, —C(O)N(R^{A})₂, cyano, or halogen;
      each R^{A} is independently H, C₁-C₆ alkyl, or C₃-C₆ cycloalkyl; and
R³ is C₁-C₃ alkyl optionally substituted, polysubstituted or persubstituted with fluoro, C₃-C₆ cycloalkyl, C₁-C₃ alkenyl, C₂-C₃ alkynyl, or C₅-C₆ cycloalkenyl;
R⁴ is H, halogen, C₁-C₃ alkyl, C₃-C₆ cycloalkyl, C₁-C₃ alkenyl, C₂-C₃ alkynyl, or C₅-C₆ cycloalkenyl or cyano, wherein each C₁-C₃ alkyl and C₃-C₆ cycloalkyl is optionally substituted with 1-5 halo groups;
R^{H} is bromo, chloro or fluoro, preferably chloro;
R⁶ and R^{6A} independently represent H or C₁-C₆ alkyl;
R^{5A} represents NR⁷R⁸, C₁-C₆ alkyl, C₃-C₆ cycloalkyl, C₃-C₆ cycloalkyl(C₁-C₃ alkyl), phenyl or 5-6 membered heteroaryl, wherein each C₁-C₆ alkyl, C₃-C₆ cycloalkyl, C₃-C₆ cycloalkyl(C₁-C₃ alkyl), phenyl and heteroaryl is optionally substituted with 1-3 of halogen, halo(C₁-C₃ alkyl), hydroxy, C₁-C₃ alkyl, C₁-C₃ alkoxy, cyano, amino, or mono- or di(C₁-C₃) alkyl amino;
R⁷ represents H or C₁-C₃ alkyl;
R⁸ represent H, C₁-C₆ alkyl, C₃-C₆ cycloalkyl or C₃-C₆ cycloalkyl(C₁-C₃ alkyl) wherein each C₁-C₆ alkyl, C₃-C₆ cycloalkyl and C₃-C₆ cycloalkyl(C₁-C₃ alkyl) is optionally substituted with 1-3 of halogen, halo(C₁-C₃ alkyl), hydroxy, C₁-C₃ alkyl, C₁-C₃ alkoxy, cyano, amino, or mono- or di(C₁-C₃) alkyl amino; or
R⁷ and R⁸ together with the nitrogen to which they are attached form ring Q, wherein ring Q is
   a 3-8 membered monocyclic heterocyclyl group, wherein the heterocyclyl group
      is optionally fused to an aromatic or non-aromatic ring containing 3-6 ring members of which 1 or 2 are optionally nitrogen, oxygen or sulfur atoms and the remainder carbons, or
      with a carbon atom from the monocyclic heterocyclyl group and additional carbon or hetero atoms, forms a spirocyclic ring system having up to 10 total ring members, wherein the ring members are carbons or 1 or 2 hetero atoms selected from nitrogen, sulfur and oxygen atoms, and
         wherein each ring Q is optionally substituted, on an aromatic or non-aromatic portion, with 1-4 R⁹ groups, wherein each R⁹ is independently (i) C₁-C₆ alkyl, (ii) C₁-C₆ alkoxy, (iii) C₃-C₆ cycloalkyl, (iv) cyano, (ii) hydroxy, (v) oxo, (vi) halogen, (vii) halo C₁-C₃ alkyl, (viii) hydroxy C₁-C₆ alkyl, (ix) amino, (x) mono- or di-(C₁-C₆ alkyl) amino, (xi) C₂-C₆ alkanoyl optionally substituted with 1 or 2 of C₁-C₃ alkyl, C₁-C₃ alkoxy, C₃-C₆ cycloalkyl, cyano, hydroxy, oxo, halogen, halo C₁-C₃ alkyl, hydroxy C₁-C₃ alkyl, amino, or mono- or di-(C₁-C₆ alkyl) amino, wherein each C₁-C₃ alkyl or C₃-C₆ cycloalkyl on the alkanoyl is optionally substituted with halogen, hydroxy or amino, or (xii) C₃-C₆ cycloalkyloxy, or (xiii) phenyl, phenyl(C₁-C₃ alkanoyl), 5-7 membered heterocyclyl, or 5-6 membered heteroaryl where each phenyl, 5-7 membered heterocyclyl and 5-6 membered heteroaryl is optionally substituted with one to four C₁-C₃ alkyl, C₁-C₃ alkoxy, halogen, hydroxy, cyano, oxo, amino, or mono- or di(C₁-C₃ alkyl)amino.

In certain embodiments of formula (IV-A)-formula (IV-E) as otherwise described herein R^{5A} represents NR⁷R⁸, and R⁷ and R⁸ together with the nitrogen to which they are attached form ring Q, wherein ring Q is
a 3-7 membered monocyclic heterocyclyl group, and
   the heterocyclyl group is optionally substituted with 1-4 R⁹ groups, wherein each R⁹ is independently C₁-C₆ alkyl, C₁-C₆ alkoxy, C₃-C₆ cycloalkyl, cyano, hydroxy, oxo, halogen, halo C₁-C₃ alkyl, hydroxy C₁-C₆ alkyl, amino, mono- or di-(C₁-C₆ alkyl) amino, or phenyl or phenyl(C₁-C₃ alkanoyl) where each phenyl is optionally substituted with C₁-C₃ alkyl, C₁-C₃ alkoxy, halogen, hydroxy, cyano, amino, or mono- or di(C₁-C₃ alkyl)amino (Embodiment (IV-AE-1)).

In certain embodiments of formula (IV-A)-formula (IV-E) as otherwise described herein R^{5A} represents piperidinyl, piperazinyl, or morpholinyl, each of which is optionally substituted with 1-4 R⁹ groups, wherein each R⁹ is independently C₁-C₆ alkyl, C₁-C₆ alkoxy, C₃-C₆ cycloalkyl, cyano, hydroxy, oxo, halogen, halo C₁-C₃ alkyl, hydroxy C₁-C₆ alkyl, amino, mono- or di-(C₁-C₆ alkyl) amino, or phenyl or phenyl(C₁-C₃ alkanoyl) where each phenyl is optionally substituted with C₁-C₃ alkyl, C₁-C₃ alkoxy, halogen, hydroxy, cyano, amino, or mono- or di(C₁-C₃ alkyl)amino (Embodiment (IV-AE-2)).

In particular embodiments of Embodiments (IV-AE-1)) and (IV-AE-2), R² is phenyl or pyridinyl, each of which is optionally substituted with 1-5 R¹², and at least one R¹² group is —C(O)OR^{A}, where R^{A} is H, methyl or ethyl.

In particular embodiments of Embodiments (IV-AE-1)) and (IV-AE-2), R² is phenyl or pyridinyl, each of which is optionally substituted with 1-5 R¹², and at least one R¹² group is —C(O)OR^{A}, where R^{A} is H.

In particular embodiments of Embodiments (IV-AE-1)) and (IV-AE-2), R² is phenyl or pyridinyl, each of which is optionally substituted with 1-5 R¹², and at least one R¹² group is —C(O)OR^{A}, where R^{A} is methyl or ethyl.

In particular embodiments of Embodiments (IV-AE-1)) and (IV-AE-2), R² is phenyl which is optionally substituted with 1-5 R¹².

In particular embodiments of Embodiments (IV-AE-1)) and (IV-AE-2), R² is phenyl which is optionally substituted with 1-5 R¹², and at least one R¹² group is —C(O)OR^{A}.

In particular embodiments of Embodiments (IV-AE-1)) and (IV-AE-2), R² is phenyl which is optionally substituted with 1-5 R¹², and at least one R¹² group is —C(O)OR^{A}, where R^{A} is H, methyl or ethyl.

In particular embodiments of Embodiments (IV-AE-1)) and (IV-AE-2), R² is phenyl which is optionally substituted with 1-5 R¹², and at least one R¹² group is —C(O)OR^{A}, where R^{A} is H.

In particular embodiments of Embodiments (IV-AE-1)) and (IV-AE-2), R² is phenyl which is optionally substituted with 1-5 R¹², and at least one R¹² group is —C(O)OR^{A}, where R^{A} is methyl or ethyl.

In particular embodiments of Embodiments (IV-AE-1)) and (IV-AE-2), R² is pyridinyl which is optionally substituted with 1-5 R¹².

In particular embodiments of Embodiments (IV-AE-1)) and (IV-AE-2), R² is pyridinyl which is optionally substituted with 1-5 R¹², and at least one R¹² group is —C(O)OR^{A}.

In particular embodiments of Embodiments (IV-AE-1)) and (IV-AE-2), R² is pyridinyl which is optionally substituted with 1-5 R¹², and at least one R¹² group is —C(O)OR^{A}, where R^{A} is H, methyl or ethyl.

In particular embodiments of Embodiments (IV-AE-1)) and (IV-AE-2), R² is pyridinyl which is optionally substituted with 1-5 R¹², and at least one R¹² group is —C(O)OR^{A}, where R^{A} is H.

In particular embodiments of Embodiments (IV-AE-1)) and (IV-AE-2), R² is pyridinyl which is optionally substituted with 1-5 R¹², and at least one R¹² group is —C(O)OR^{A}, where R^{A} is methyl or ethyl.

In particular embodiments of Embodiments (IV-AE-1)) and (IV-AE-2), R² is a group of the formula

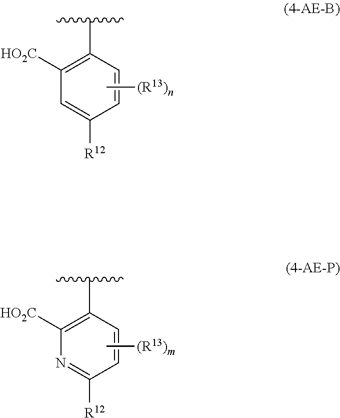

wherein
each R¹³ is independently C₁-C₄ alkyl, —OR^{B}, —C(O)OR^{B} (C₁-C₃ alkyl)-OR^{B}, —C(O)N(R^{B})₂, cyano, or halogen;
   each R^{B} is independently H, C₁-C₆ alkyl, or C₃-C₆ cycloalkyl;
R¹² is hydrogen, C₁-C₄ alkyl, —OR^{A}, —C(O)OR^{A}, (C₁-C₃ alkyl)-OR^{A}, —C(O)N(R^{A})₂, cyano, or halogen;
   each R^{A} is independently H, C₁-C₆ alkyl, or C₃-C₆ cycloalkyl;
n is 0, 1, 2 or 3; and
m is 0, 1 or 2.

In certain embodiments of formula (4-AE-B) and formula (4-AE-P), R¹² is cyano or halogen. In other embodiments of formula (4-AE-B) and formula (4-AE-P), R¹² is cyano. In other embodiments of formula (4-AE-B) or (4-AE-P), R¹² is fluoro, chloro or bromo. In other embodiments of formula (4-AE-B) or (4-AE-P), R¹² is fluoro or chloro. In other embodiments of formula 4-AE-B) or (4-AE-P), R¹² is methyl. In other embodiments of formula (4-AE-B) or (4-AE-P), R¹² is hydrogen.

In certain embodiments of formula (I) or formula (IV) as otherwise described herein the compound is of formula (II) or (III)

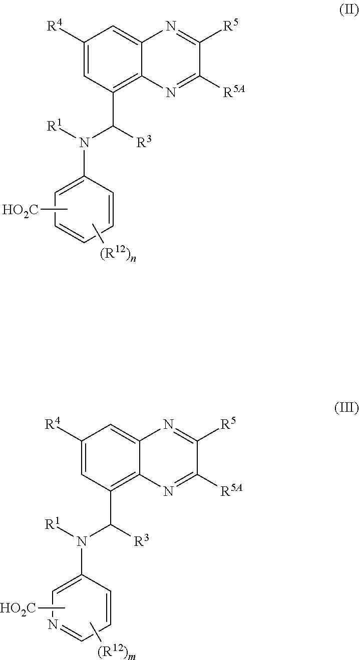

wherein n is 0, 1, 2, 3 or 4; and
m is 0, 1, 2 or 3.

In certain embodiments of formula (II) or formula (III) as otherwise described herein the compound is of formula (II-A)

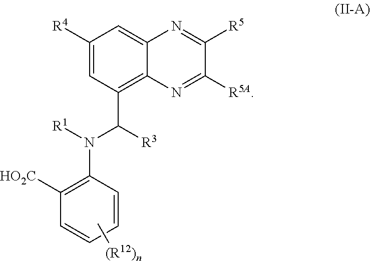

In certain embodiments of formula (II) or formula (III) as otherwise described herein the compound is of formula (III-A)

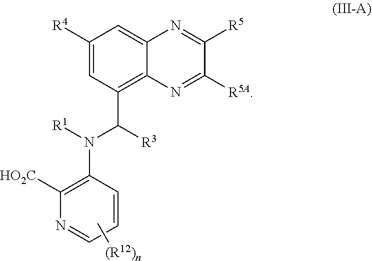

In certain embodiments of formula (II), formula (III), formula (II-A) or formula (III-A) as otherwise described herein:
R¹ is H, C₁-C₃ alkyl, or C₃-C₆ cycloalkyl;
   each R¹² is independently C₁-C₄ alkyl; —OR^{A}, —C(O)OR^{A}, (1-C₃ alkyl)-OR^{A}, —C(O)N(R^{A})₂, cyano, or halogen;
      each R^{A} is independently H, C₁-C₆ alkyl, or C₃-C₆ cycloalkyl; and
R³ is C₁-C₃ alkyl or C₃-C₆ cycloalkyl; and
R⁴ is H, C₁-C₃ alkyl, C₃-C₆ cycloalkyl, cyano, or halo, wherein each C₁-C₃ alkyl is optionally substituted with 1-5 halo groups.

In certain embodiments of formula (II), formula (III), formula (II-A) or formula (III-A) as otherwise described herein R³ is C₁-C₃ alkyl.

In certain embodiments of formula (II), formula (III), formula (II-A) or formula (III-A) as otherwise described herein R³ is methyl.

In certain embodiments of formula (II), formula (III), formula (II-A) or formula (III-A) as otherwise described herein R⁴ is H, C₁-C₃ alkyl, C₃-C₄ cycloalkyl or cyano.

In certain embodiments of formula (II), formula (III), formula (II-A) or formula (III-A) as otherwise described herein R⁷ is hydrogen and R^{a} represents hydrogen, C₁-C₆ alkyl, C₃-C₆ cycloalkyl or C₃-C₆ cycloalkyl(C₁-C₃ alkyl).

In certain embodiments of formula (II), formula (III), formula (II-A) or formula (III-A) as otherwise described herein R^{5A} represents NR⁷R⁸ and R⁷ and R⁸ together with the nitrogen to which they are attached form ring Q, and ring Q is a C₁-C₆ heterocyclyl

In certain embodiments of formula (II), formula (III), formula (II-A) or formula (III-A) as otherwise described herein R^{5A} represents NR⁷R⁸ and R⁷ and R⁸ together with the nitrogen to which they are attached form ring Q, and ring Q is aziridinyl, azetidinyl, pyrrolidinyl, piperazinyl, morpholinyl, piperidinyl, or azepanyl, each of which is optionally substituted with 1-2 R⁹ groups, wherein each R⁹ is independently C₁-C₆ alkyl, C₁-C₆ alkoxy, C₃-C₆ cycloalkyl, cyano, hydroxy, oxo, halogen, halo C₁-C₃ alkyl, hydroxy C₁-C₆ alkyl, amino, mono- or di-(C₁-C₆ alkyl) amino, phenyl, 5-7 membered heterocyclyl, or 5-6 membered heteroaryl where each phenyl, 5-7 membered heterocyclyl and 5-6 membered heteroaryl is optionally substituted with one to four C₁-C₃ alkyl, C₁-C₃ alkoxy, halogen, hydroxy, cyano, amino, or mono- or di(C₁-C₃ alkyl)amino.

In certain embodiments of formula (II), formula (III), formula (II-A) or formula (III-A) as otherwise described herein R^{5A} represents NR⁷R⁸ and R⁷ and R⁸ together with the nitrogen to which they are attached form ring Q, and ring Q is aziridinyl, azetidinyl, pyrrolidinyl, piperidinyl, piperazinyl, morpholinyl, or azepanyl, each of which is optionally substituted with 1, 2, 3 or 4 halogens.

In certain embodiments of formula (II), formula (III), formula (II-A) or formula (III-A) as otherwise described herein R^{5A} represents NR⁷R⁸ and R⁷ and R⁸ together with the nitrogen to which they are attached form ring Q, and ring Q is a 5-6 membered monocyclic heterocyclyl group fused to an aromatic or non-aromatic ring containing 3-6 ring members of which 1 or 2 are optionally nitrogen atoms and the remainder carbons, and the fused ring system is optionally substituted, on either ring, with 1-4 R⁹ groups, wherein each R⁹ is independently C₁-C₆ alkyl, C₁-C₆ alkoxy, C₃-C₆ cycloalkyl, cyano, hydroxy, oxo, halogen, halo C₁-C₃ alkyl, hydroxy C₁-C₆ alkyl, amino, or mono- or di-(C₁-C₆ alkyl) amino.

In certain embodiments of formula (II), formula (III), formula (II-A) or formula (III-A) as otherwise described herein R^{5A} represents NR⁷R⁸ and R⁷ and R⁸ together with the nitrogen to which they are attached form ring Q, where ring Q is a group of the formula:

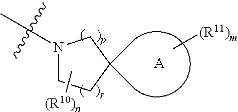

wherein
   each ring A is a 3-6 membered carbocyclic ring or 3-6 membered heterocyclic ring,
   m is 0, 1, 2, 3, or 4;
   n is 0, 1 or 2;
   p and r are independently 1 or 2, provided that the sum of p and r is 2 or 3;
   each R¹⁰ and R¹¹ is independently C₁-C₆ alkyl, C₁-C₆ alkoxy, halogen, cyano, hydroxy, hydroxy C₁-C₆ alkyl, amino, mono- or di(C₁-C₆ alkyl) amino, C₃-C₆ cycloalkyl, phenyl or 5-6 membered heteroaryl, where each cycloalkyl, phenyl and heteroaryl is optionally substituted with 1-3 of halogen, hydroxy, C₁-C₃ alkyl, C₁-C₃ alkoxy, cyano, amino, or mono- or di(C₁-C₃) alkyl amino.

In certain embodiments of formula (II), formula (III), formula (II-A) or formula (III-A) as otherwise described herein R⁷ and R⁸ together with the nitrogen to which they are attached form a piperidinyl, piperazinyl or morpholinyl group, each of which is optionally substituted with 1-4 R⁹ groups, wherein each R⁹ is independently C₁-C₆ alkyl, C₁-C₆ alkoxy, C₃-C₆ cycloalkyl, cyano, hydroxy, oxo, halogen, halo C₁-C₃ alkyl, hydroxy C₁-C₆ alkyl, amino, mono- or di-(C₁-C₆ alkyl) amino, or phenyl or phenyl(C₁-C₃ alkanoyl) where each phenyl is optionally substituted with C₁-C₃ alkyl, C₁-C₃ alkoxy, halogen, hydroxy, cyano, amino, or mono- or di(C₁-C₃ alkyl)amino.

In certain embodiments of formula (II), formula (III), formula (II-A) or formula (III-A) as otherwise described herein R⁷ and R⁸ together with the nitrogen to which they are attached form a piperidinyl, piperazinyl or morpholinyl group, each of which is optionally substituted with 1-4 R⁹ groups, wherein each R⁹ is independently C₁-C₃ alkyl, C₁-C₃ alkoxy, C₃-C₆ cycloalkyl, cyano, hydroxy, oxo, halogen, halo C₁-C₃ alkyl, hydroxy C₁-C₃ alkyl, amino, or mono- or di-(C₁-C₃ alkyl) amino.

In certain embodiments of formula (II), formula (III), formula (II-A) or formula (III-A) as otherwise described herein R⁷ and R⁸ together with the nitrogen to which they are attached form a piperidinyl, piperazinyl or morpholinyl group, each of which is optionally substituted with 1-4 R⁹ groups, wherein each R⁹ is independently methyl, ethyl, methoxy, ethoxy, cyano, fluoro, chloro, trifluoromethyl, cyclopropyl, or cyclopropylmethyl.

In certain embodiments of formula (I), formula (II), formula (III), formula (II-A) or formula (III-A) as otherwise described herein R⁷ and R⁸ together with the nitrogen to which they are attached form ring Q, wherein ring Q represents a nitrogen-containing ring selected from

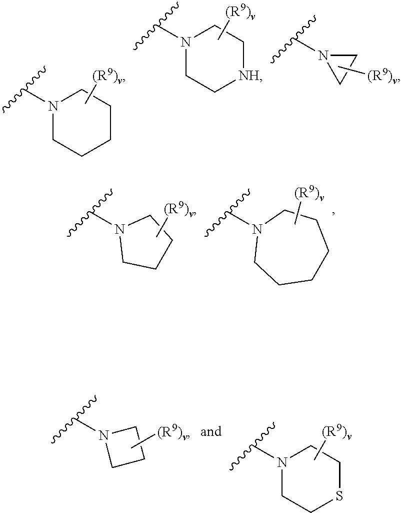

wherein each v represents 0, 1, 2, 3 or 4.

In certain embodiments, the nitrogen-containing ring is substituted independently with 1, 2 or 3 halogens. In certain embodiments, the halogens are independently fluorine atoms or chlorine atoms.

In certain embodiments, the nitrogen-containing ring is substituted with phenyl or phenyl(C₁-C₃ alkanoyl) where each phenyl is optionally substituted with C₁-C₃ alkyl, C₁-C₃ alkoxy, halogen, hydroxy, cyano, amino, or mono- or di(C₁-C₃ alkyl)amino.

In certain embodiments of formula (I), formula (II), formula (III), formula (II-A) or formula (III-A) as otherwise described herein R^{5A} represents a group of the formula

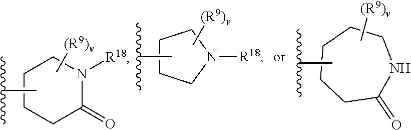

wherein
   each v represents 0, 1, 2, 3 or 4; and
   R¹⁸ represents H or C₁-C₆ alkyl.

In certain embodiments, the nitrogen-containing ring is substituted independently with 1, 2 or 3 halogens. In certain embodiments, the halogens are independently fluorine atoms or chlorine atoms.

In certain embodiments, the nitrogen-containing ring is substituted with phenyl or phenyl(C₁-C₃ alkanoyl) where each phenyl is optionally substituted with C₁-C₃ alkyl, C₁-C₃ alkoxy, halogen, hydroxy, cyano, amino, or mono- or di(C₁-C₃ alkyl)amino.

In certain embodiments of formula (I) or formula (IV) as otherwise described herein, R⁷ and R⁸ together with the nitrogen to which they are attached form ring Q, wherein ring Q is
a 3-7 membered monocyclic heterocyclyl group containing one or two ring nitrogens and only nitrogen heteroatoms, and
   the heterocyclyl group is fused to an aromatic or non-aromatic ring containing 3-6 ring members of which 1 or 2 are optionally nitrogen atoms and the remainder carbons, wherein
      the fused heterocyclyl group is optionally substituted, on an aromatic or non-aromatic portion, with 1-4 R⁹ groups, wherein each R⁹ is independently C₁-C₆ alkyl, C₁-C₆ alkoxy, C₃-C₆ cycloalkyl, cyano, hydroxy, oxo, halogen, halo C₁-C₃ alkyl, hydroxy C₁-C₆ alkyl, amino, mono- or di-(C₁-C₆ alkyl) amino, or phenyl or phenyl(C₁-C₃ alkanoyl) where each phenyl is optionally substituted with C₁-C₃ alkyl, C₁-C₃ alkoxy, halogen, hydroxy, cyano, amino, or mono- or di(C₁-C₃ alkyl)amino.

In certain embodiments of formula (I) or formula (IV) as otherwise described herein, R⁷ and R⁸ together with the nitrogen to which they are attached form ring Q, wherein ring Q represents a fused bicyclic group selected from

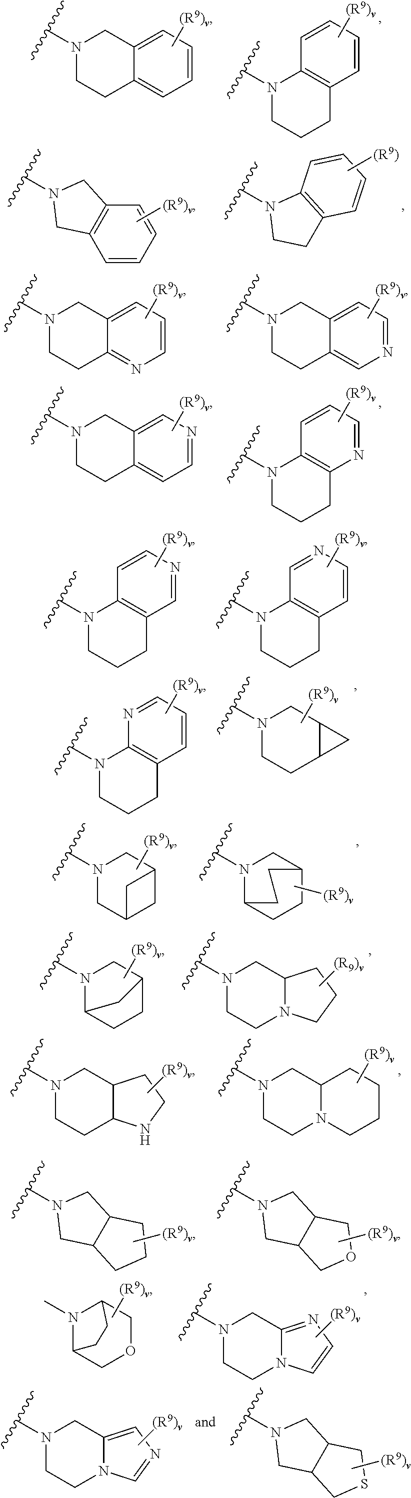

wherein each v represents 0, 1, 2, 3 or 4 and the R⁹ groups can be on either ring.

In certain embodiments, the fused bicyclic group is substituted independently with 1, 2, 3 or 4 of C₁-C₄ alkyl, C₃-C₆ cycloalkyl, C₁-C₃ haloalkyl or halogen.

In certain embodiments, the fused bicyclic group is substituted independently with 1, 2, 3 or 4 halogens. In certain embodiments, the halogens are independently fluorine atoms or chlorine atoms. In other embodiments, the halogens are fluorine atoms.

In other embodiments, the fused bicyclic group is substituted with phenyl or phenyl(C₁-C₃ alkanoyl) where each phenyl is optionally substituted with C₁-C₃ alkyl, C₁-C₃ alkoxy, halogen, hydroxy, cyano, amino, or mono- or di(C₁-C₃ alkyl)amino.

In certain embodiments of formula (I) or formula (IV) as otherwise described herein, R⁷ and R⁸ together with the nitrogen to which they are attached form a 3-7 membered monocyclic heterocyclyl group containing one or two ring nitrogens and only nitrogen heteroatoms, and
the heterocyclyl group forms a spirocyclic ring system having up to 10 total ring members with a carbon atom from the monocyclic heterocyclyl group and additional carbon, nitrogen, oxygen or sulfur atoms, wherein
   wherein the spirocyclic ring system is optionally substituted with 1-4 R⁹ groups, wherein each R⁹ is independently C₁-C₆ alkyl, C₁-C₆ alkoxy, C₃-C₆ cycloalkyl, cyano, hydroxy, oxo, halogen, halo C₁-C₃ alkyl, hydroxy C₁-C₆ alkyl, amino, mono- or di-(C₁-C₆ alkyl) amino, or phenyl or phenyl(C₁-C₃ alkanoyl) where each phenyl is optionally substituted with C₁-C₃ alkyl, C₁-C₃ alkoxy, halogen, hydroxy, cyano, amino, or mono- or di(C₁-C₃ alkyl)amino.

In certain embodiments of formula (I) or formula (IV) as otherwise described herein, R⁷ and R⁸ together with the nitrogen to which they are attached represent a spirocyclic ring system having one or two heteroatoms selected from nitrogen, oxygen and sulfur, preferably nitrogen and oxygen, and up to 10 total ring members. The spirocyclic ring system is optionally substituted with up to 4 R⁹ groups. Examples of such optionally substituted spirocyclic ring systems are

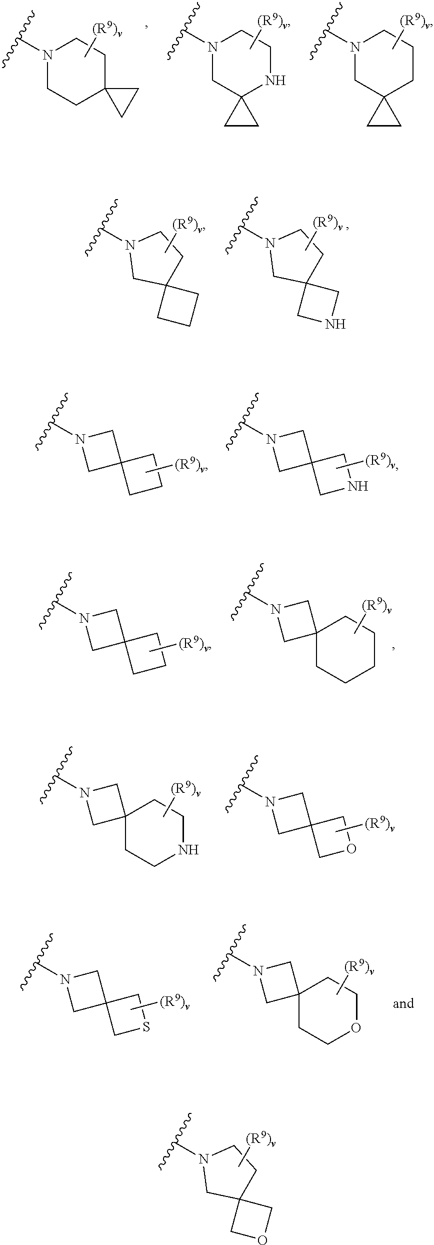

wherein each v represents 0, 1, 2, 3 or 4 and the R⁹ groups can be on either ring.

In certain embodiments, the spirocyclic ring system is substituted independently with 1, 2, 3 or 4 of C₁-C₄ alkyl, C₃-C₆ cycloalkyl, C₁-C₃ haloalkyl or halogen.

In certain embodiments, the spirocyclic ring system is substituted independently with 1, 2, 3 or 4 halogens. In certain embodiments, the halogens are independently fluorine atoms or chlorine atoms. In other embodiments, the halogens are fluorine atoms.

In certain embodiments, the spirocyclic ring system is substituted with phenyl or phenyl(C₁-C₃ alkanoyl) where each phenyl is optionally substituted with C₁-C₃ alkyl, C₁-C₃ alkoxy, halogen, hydroxy, cyano, amino, or mono- or di(C₁-C₃ alkyl)amino.

In certain embodiments of formula (I), formula (II), formula (III), formula (IV), formula (II-A) or formula (III-A) as otherwise described herein R⁹ is an optionally substituted 5-6 membered heteroaryl group. Examples of suitable heteroaryl groups are pyrazolyl, thiazolyl, imidazolyl, pyridinyl, oxazolyl, and isoxazolyl, each of which is optionally substituted with one to four C₁-C₃ alkyl, C₁-C₃ alkoxy, halogen, hydroxy, cyano, oxo, amino, or mono- or di(C₁-C₃ alkyl)amino.

In certain embodiments, the stereochemical orientation at R³ is such that the compound will have an “R” stereochemical designation at that position according to the Cahn-Ingold-Prelog priority rules. Thus, for example, the compound is of formula (I-1):

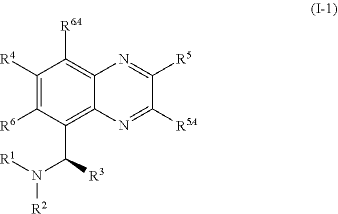

where the groups are defined as above for formula (I).

In certain embodiments of formula (I) or formula (IV) as otherwise described herein the compound is of formula (II-A-1) or (III-A-1)

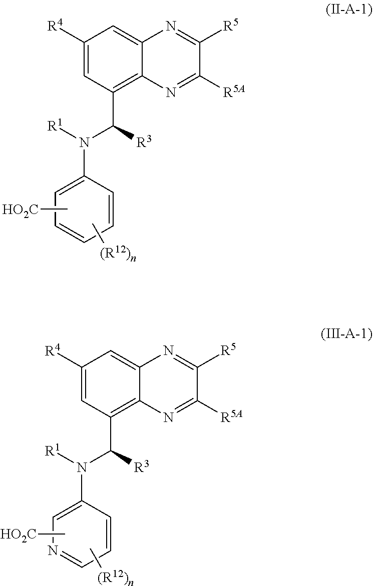

wherein n is 0, 1, 2, 3 or 4; and
m is 0, 1, 2 or 3.

In certain embodiments of formula (I), the compound is of formula (II-A-1.1) or (III-A-1.1)

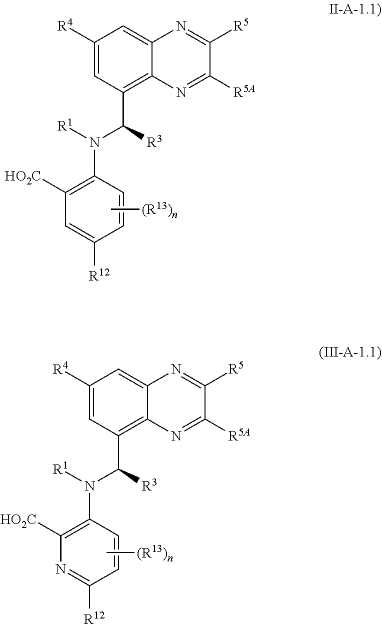

wherein
each R¹³ is independently C₁-C₄ alkyl, —OR^{B}, —C(O)OR^{B} (1-C₃ alkyl)-OR^{B}, —C(O)N(R^{B})₂, cyano, or halogen;
   each R^{B} is independently H, C₁-C₆ alkyl, or C₃-C₆ cycloalkyl;
R¹² is hydrogen, C₁-C₄ alkyl, —OR^{A}, —C(O)OR^{A}, (C₁-C₃ alkyl)-OR^{A}, —C(O)N(R^{A})₂, cyano, or halogen;
   each R^{A} is independently H, C₁-C₆ alkyl, or C₃-C₆ cycloalkyl;
n is 0, 1, 2 or 3; and
m is 0, 1 or 2.

In certain embodiments of formula (II-A-1.1) and formula (III-A-1.1), R¹² is cyano or halogen. In other embodiments of formula (II-A-1.1) and formula (III-A-1.1), R¹² is cyano. In other embodiments of formula (II-A-1.1) or (III-A-1.1), R¹² is fluoro, chloro or bromo. In other embodiments of formula (II-A-1.1) or (III-A-1.1), R¹² is fluoro or chloro. In other embodiments of formula (II-A-1.1) or (III-A-1.1), R¹² is methyl. In other embodiments of formula (II-A-1.1) or (III-A-1.1), R¹² is hydrogen.

In certain embodiments of formula (II-A-1.1) and formula (III-A-1.1), n and m are 1, R¹³ is hydrogen, methyl or ethyl, and R¹² is cyano or halogen. In other embodiments of formula (II-A-1.1) and formula (III-A-1.1), n and m are 1, R¹³ is hydrogen, methyl or ethyl, and R¹² is cyano. In other embodiments of formula (II-A-1.1) or (III-A-1.1), n and m are 1, R¹³ is hydrogen, methyl or ethyl, and R¹² is fluoro, chloro or bromo. In other embodiments of formula (II-A-1.1) or (III-A-1.1), n and m are 1, R¹³ is hydrogen, methyl or ethyl, and R¹² is fluoro or chloro.

In certain embodiments of formula (II-A-1.1) and formula (III-A-1.1), R⁵ is methyl, n and m are 1, R¹³ is hydrogen, methyl or ethyl,

and
i. R¹² is cyano or halogen;
ii. R¹² is cyano;
iii. R¹² is fluoro, chloro or bromo; or
iv. R¹² is fluoro or chloro.

In certain embodiments of formula (II-A-1.1) and formula (III-A-1.1), R⁵ is ethyl, n and m are 1, R¹³ is hydrogen, methyl or ethyl, and
i. R¹² is cyano or halogen;
ii. R¹² is cyano;
iii. R¹² is fluoro, chloro or bromo; or
iv. R¹² is fluoro or chloro.

In certain embodiments of formula (II-A-1.1) and formula (III-A-1.1), R⁵ is cyano, n and m are 1, R¹³ is hydrogen, methyl or ethyl, and
i. R¹² is cyano or halogen;
ii. R¹² is cyano;
iii. R¹² is fluoro, chloro or bromo; or
iv. R¹² is fluoro or chloro.

In certain embodiments of formula (II-A-1.1) and formula (III-A-1.1), R⁵ is cyclopropyl, n and m are 1, R¹³ is hydrogen, methyl or ethyl, and
i. R¹² is cyano or halogen;
ii. R¹² is cyano;
iii. R¹² is fluoro, chloro or bromo; or
iv. R¹² is fluoro or chloro.

In certain embodiments of formula (II-A-1.1) and formula (III-A-1.1), R⁵ is bromo, n and m are 1, R¹³ is hydrogen, methyl or ethyl, and
i. R¹² is cyano or halogen;
ii. R¹² is cyano;
iii. R¹² is fluoro, chloro or bromo; or
iv. R¹² is fluoro or chloro.

In certain embodiments of formula (II-A-1.1) and formula (III-A-1.1), R⁵ is fluoro, n and m are 1, R¹³ is hydrogen, methyl or ethyl, and
i. R¹² is cyano or halogen;
ii. R¹² is cyano;
iii. R¹² is fluoro, chloro or bromo; or
iv. R¹² is fluoro or chloro.

In certain embodiments of formula (II-A-1.1) and formula (III-A-1.1), R⁵ is chloro, n and m are 1, R¹³ is hydrogen, methyl or ethyl, and
i. R¹² is cyano or halogen;
ii. R¹² is cyano;
iii. R¹² is fluoro, chloro or bromo; or
iv. R¹² is fluoro or chloro.

In certain embodiments of formula (II-A-1.1) and formula (III-A-1.1), R⁵ is trifluoromethyl, n and m are 1, R¹³ is hydrogen, methyl or ethyl, and
i. R¹² is cyano or halogen;
ii. R¹² is cyano;
iii. R¹² is fluoro, chloro or bromo; or
iv. R¹² is fluoro or chloro.

In certain embodiments of formula (I), R⁵ is a 3-8 membered heterocyclyl group. In these embodiments, the heterocyclyl group can be connected to the parent ring system via a carbon atom in the heterocyclyl ring. Examples of such groups are tetrahydrofuran-3-yl, tetrahydro-2H-pyran-2-yl, morpholin-2-yl, piperidin-4-yl, 3,6-dihydro-2H-pyran-4-yl, and tetrahydro-2H-pyran-4-yl.

In one embodiment, the compound of Formula (I) or Formula (IV) is selected from:

| Name | Structure |
|---|---|
| 2-((1-(2-cyano-7-methyl-3-(piperidin-1- yl)quinoxalin-5-yl)ethyl)amino)benzoic acid | 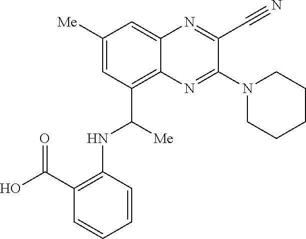 [SMILES: Cc1cc(C(C)Nc2ccccc2C(=O)O)c2nc(N3CCCCC3)c(C#N)nc2c1] |
| 2-((1-(2-cyano-3-(isoindolin-2-yl)-7- methylquinoxalin-5-yl)ethyl)amino)benzoic acid | 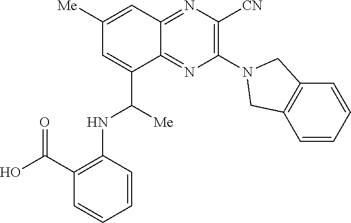 [SMILES: Cc1cc(C(C)Nc2ccccc2C(=O)O)c2nc(N3Cc4ccccc4C3)c(C#N)nc2c1] |
| 2-((1-(2-cyano-3-(4,4-difluoropiperidin-1-yl)-7- methylquinoxalin-5-yl)ethyl)amino)benzoic acid | 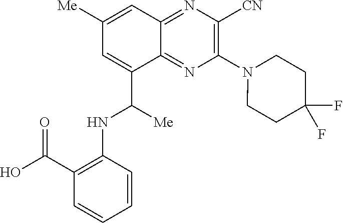 [SMILES: Cc1cc(C(C)Nc2ccccc2C(=O)O)c2nc(N3CCC(F)(F)CC3)c(C#N)nc2c1] |
| 6-chloro-3-((1-(2-cyano-3-(4,4-difluoropiperidin- 1-yl)-7-methylquinoxalin-5-yl)ethyl)amino)picolinic acid | 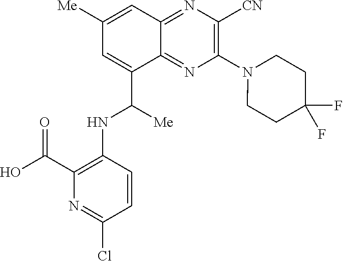 [SMILES: Cc1cc(C(C)Nc2ccc(Cl)nc2C(=O)O)c2nc(N3CCC(F)(F)CC3)c(C#N)nc2c1] |
| 2-((1-(2-cyano-3-(isoindolin-2-yl)-7- methylquinoxalin-5-yl)ethyl)amino)benzoic acid | 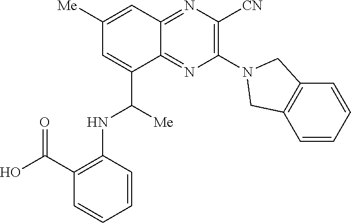 [SMILES: Cc1cc(C(C)Nc2ccccc2C(=O)O)c2nc(N3Cc4ccccc4C3)c(C#N)nc2c1] |
| 6-chloro-3-((1-(2-cyano-3-(6,6-difluoro-3- azabicyclo[3.1.1]heptan-3-yl)-7-methylquinoxalin-5- yl)ethyl)amino)picolinic acid | 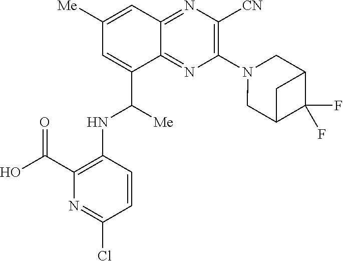 [SMILES: Cc1cc(C(C)Nc2ccc(Cl)nc2C(=O)O)c2nc(N3CC4CC(C3)C4(F)F)c(C#N)nc2c1] |
| 6-chloro-3-((1-(2-cyano-3-(6,6-difluoro-2- azabicyclo[2.2.1]heptan-2-yl)-7-methylquinoxalin-5- yl)ethyl)amino)picolinic acid | 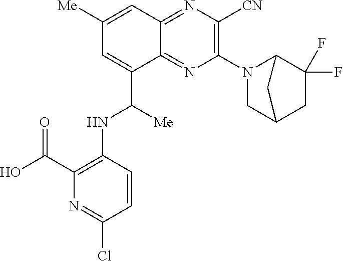 [SMILES: Cc1cc(C(C)Nc2ccc(Cl)nc2C(=O)O)c2nc(N3CC4CC3C(F)(F)C4)c(C#N)nc2c1] |
| 6-chloro-3-((1-(2-cyano-3-(3,3-difluoropiperidin- 1-yl)-7-methylquinoxalin-5-yl)ethyl)amino)picolinic acid | 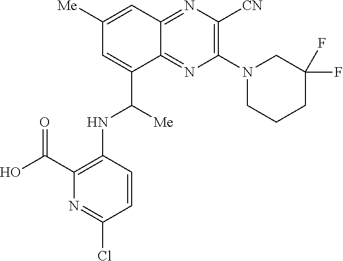 [SMILES: Cc1cc(C(C)Nc2ccc(Cl)nc2C(=O)O)c2nc(N3CCCC(F)(F)C3)c(C#N)nc2c1] |
| 6-chloro-3-((1-(2-cyano-3-(((2,2- difluorocyclopropyl)methyl)amino)-7- methylquinoxalin-5-yl)ethyl)amino)picolinic acid | 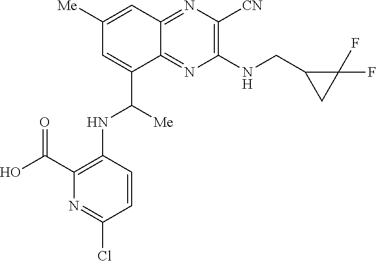 [SMILES: Cc1cc(C(C)Nc2ccc(Cl)nc2C(=O)O)c2nc(NCC3CC3(F)F)c(C#N)nc2c1] |
| 6-chloro-3-((1-(2-cyano-7-methyl-3-(((1- (trifluoromethyl)cyclopropyl)methyl)amino)quinoxalin- 5-yl)ethyl)amino)picolinic acid | 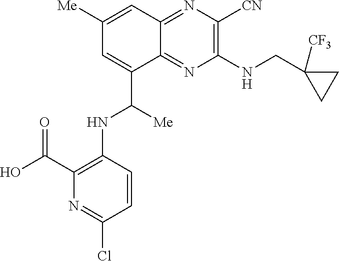 [SMILES: Cc1cc(C(C)Nc2ccc(Cl)nc2C(=O)O)c2nc(NCC3(C(F)(F)F)CC3)c(C#N)nc2c1] |
| 6-chloro-3-((1-(2-cyano-3-(1,1-difluoro-5- azaspiro[2.3]hexan-5-yl)-7-methylquinoxalin-5- yl)ethyl)amino)picolinic acid | 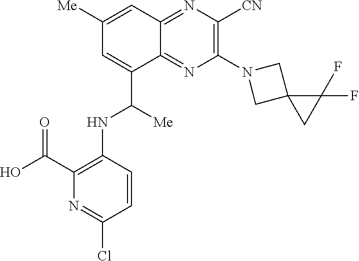 [SMILES: Cc1cc(C(C)Nc2ccc(Cl)nc2C(=O)O)c2nc(N3CC4(C3)CC4(F)F)c(C#N)nc2c1] |
| 3-((1-(2-cyano-3-(4,4-difluoropiperidin-1-yl)-7- methylquinoxalin-5-yl)ethyl)amino)-6-methylpicolinic acid | 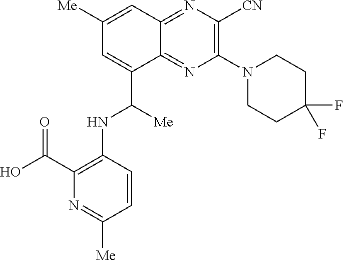 [SMILES: Cc1cc(C(C)Nc2ccc(C)nc2C(=O)O)c2nc(N3CCC(F)(F)CC3)c(C#N)nc2c1] |
| 2-((1-(2-cyano-3-((cyclopropylmethyl)amino)-7- methylquinoxalin-5-yl)ethyl)amino)benzoic acid | 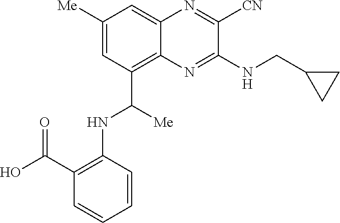 [SMILES: Cc1cc(C(C)Nc2ccccc2C(=O)O)c2nc(NCC3CC3)c(C#N)nc2c1] |
| 6-chloro-3-((1-(2-cyano-7-methyl-3-(3- (trifluoromethyl)piperidin-1-yl)quinoxalin-5- yl)ethyl)amino)picolinic acid | 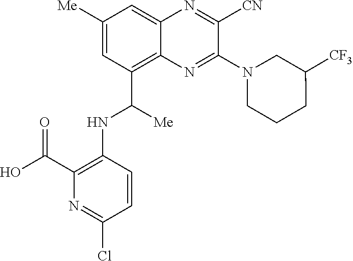 [SMILES: Cc1cc(C(C)Nc2ccc(Cl)nc2C(=O)O)c2nc(N3CCCC(C(F)(F)F)C3)c(C#N)nc2c1] |
| 6-chloro-3-((1-(2-cyano-3-(4,4-difluoropiperidin- 1-yl)-7-(trifluoromethyl)quinoxalin-5- yl)ethyl)amino)picolinic acid | 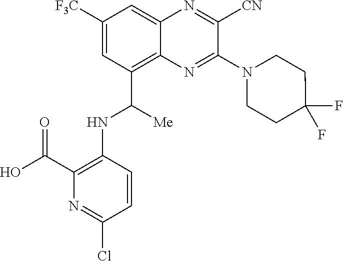 [SMILES: CC(Nc1ccc(Cl)nc1C(=O)O)c1cc(C(F)(F)F)cc2nc(C#N)c(N3CCC(F)(F)CC3)nc12] |
| 6-chloro-3-((1-(2-cyano-3-(6,6-difluoro-3- azabicyclo[3.1.0]hexan-3-yl)-7-methylquinoxalin-5- yl)ethyl)amino)picolinic acid | 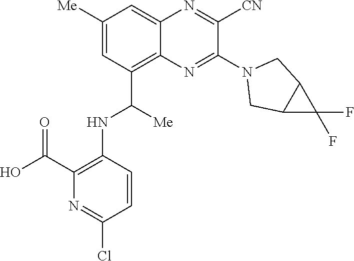 [SMILES: Cc1cc(C(C)Nc2ccc(Cl)nc2C(=O)O)c2nc(N3CC4C(C3)C4(F)F)c(C#N)nc2c1] |
| 6-chloro-3-((1-(2-cyano-3-(4,4-difluoro-6- azaspiro[2.5]octan-6-yl)-7-methylquinoxalin-5- yl)ethyl)amino)picolinic acid | 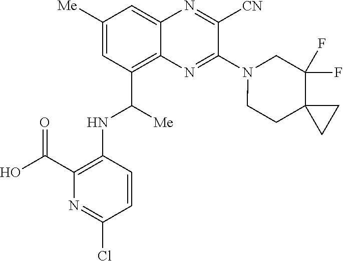 [SMILES: Cc1cc(C(C)Nc2ccc(Cl)nc2C(=O)O)c2nc(N3CCC4(CC4)C(F)(F)C3)c(C#N)nc2c1] |
| 6-chloro-3-((1-(2-cyano-3-(((1- fluorocyclopropyl)methyl)amino)-7-methylquinoxalin- 5-yl)ethyl)amino)picolinic acid | 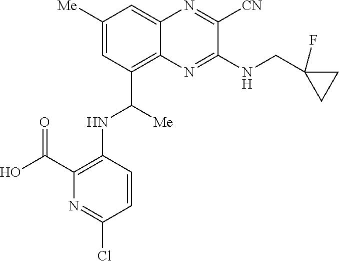 [SMILES: Cc1cc(C(C)Nc2ccc(Cl)nc2C(=O)O)c2nc(NCC3(F)CC3)c(C#N)nc2c1] |
| 6-chloro-3-((1-(2-cyano-3-(5-fluoroisoindolin-2- yl)-7-methylquinoxalin-5-yl)ethyl)amino)picolinic acid | 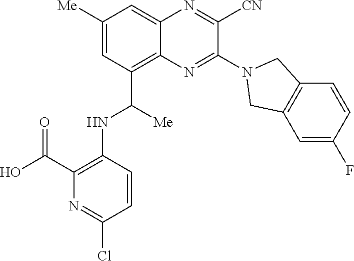 [SMILES: Cc1cc(C(C)Nc2ccc(Cl)nc2C(=O)O)c2nc(N3Cc4ccc(F)cc4C3)c(C#N)nc2c1] |
| 6-chloro-3-((1-(2-cyano-3- ((cyclopropylmethyl)amino)-7-methylquinoxalin-5- yl)ethyl)amino)picolinic acid | 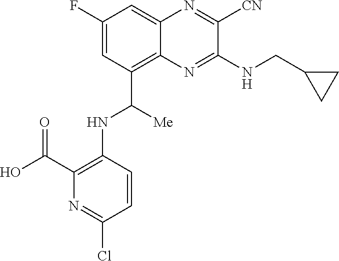 [SMILES: CC(Nc1ccc(Cl)nc1C(=O)O)c1cc(F)cc2nc(C#N)c(NCC3CC3)nc12] |
| 6-chloro-3-((1-(2-cyano-3-(4,4-difluoropiperidin- 1-yl)-7-fluoroquinoxalin-5-yl)ethyl)amino)picolinic acid | 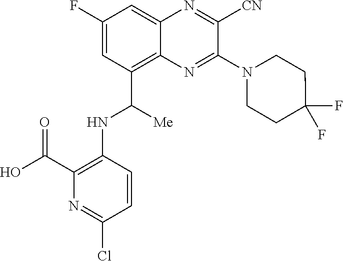 [SMILES: CC(Nc1ccc(Cl)nc1C(=O)O)c1cc(F)cc2nc(C#N)c(N3CCC(F)(F)CC3)nc12] |
| 2-((1-(2-cyano-3-(4,4-difluoropiperidin-1-yl)-7- fluoroquinoxalin-5-yl)ethyl)amino)benzoic acid | 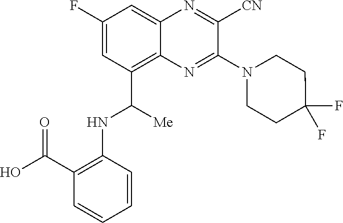 [SMILES: CC(Nc1ccccc1C(=O)O)c1cc(F)cc2nc(C#N)c(N3CCC(F)(F)CC3)nc12] |
| 2-((1-(2-cyano-7-fluoro-3-(4-fluoropiperidin-1- yl)quinoxalin-5-yl)ethyl)amino)benzoic acid | 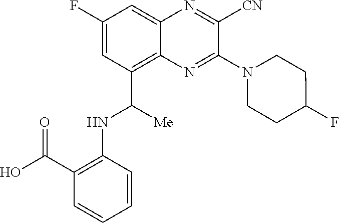 [SMILES: CC(Nc1ccccc1C(=O)O)c1cc(F)cc2nc(C#N)c(N3CCC(F)CC3)nc12] |
| 2-((1-(2,7-dicyano-3-(4-fluoropiperidin-1- yl)quinoxalin-5-yl)ethyl)amino)benzoic acid | 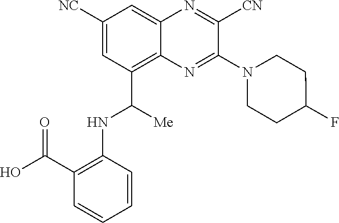 [SMILES: CC(Nc1ccccc1C(=O)O)c1cc(C#N)cc2nc(C#N)c(N3CCC(F)CC3)nc12] |
| 2-((1-(2,7-dicyano-3-(4,4-difluoropiperidin-1- yl)quinoxalin-5-yl)ethyl)amino)benzoic acid | 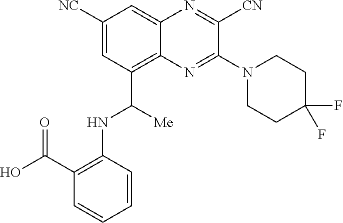 [SMILES: CC(Nc1ccccc1C(=O)O)c1cc(C#N)cc2nc(C#N)c(N3CCC(F)(F)CC3)nc12] |
| 2-((1-(7-chloro-2-cyano-3-(4,4-difluoropiperidin- 1-yl)quinoxalin-5-yl)ethyl)amino)benzoic acid | 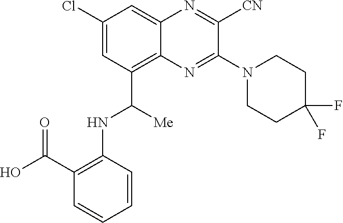 [SMILES: CC(Nc1ccccc1C(=O)O)c1cc(Cl)cc2nc(C#N)c(N3CCC(F)(F)CC3)nc12] |
| 6-chloro-3-((1-(2-cyano-7-cyclopropyl-3-(4,4- difluoropiperidin-1-yl)quinoxalin-5- yl)ethyl)amino)picolinic acid | 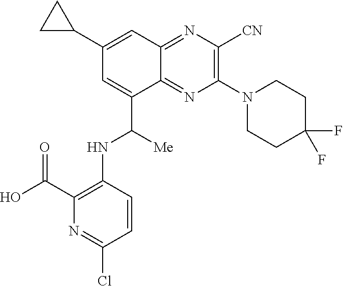 [SMILES: CC(Nc1ccc(Cl)nc1C(=O)O)c1cc(C2CC2)cc2nc(C#N)c(N3CCC(F)(F)CC3)nc12] |
| 6-chloro-3-((1-(7-chloro-2-cyano-3-(4,4- difluoropiperidin-1-yl)quinoxalin-5- yl)ethyl)amino)picolinic acid | 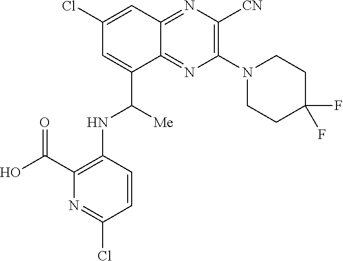 [SMILES: CC(Nc1ccc(Cl)nc1C(=O)O)c1cc(Cl)cc2nc(C#N)c(N3CCC(F)(F)CC3)nc12] |
| 6-chloro-3-((1-(3-(4,4-difluoropiperidin-1-yl)-2,7- dimethylquinoxalin-5-yl)ethyl)amino)picolinic acid | 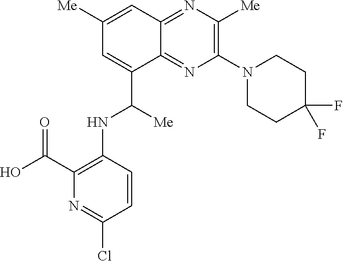 [SMILES: Cc1cc(C(C)Nc2ccc(Cl)nc2C(=O)O)c2nc(N3CCC(F)(F)CC3)c(C)nc2c1] |
| 2-((1-(3-(4,4-difluoropiperidin-1-yl)-2,7- dimethylquinoxalin-5-yl)ethyl)amino)benzoic acid | 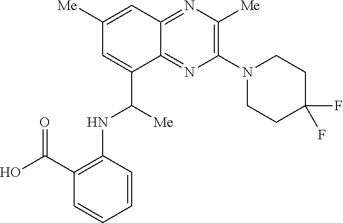 [SMILES: Cc1cc(C(C)Nc2ccccc2C(=O)O)c2nc(N3CCC(F)(F)CC3)c(C)nc2c1] |
| 2-((1-(2-cyano-7-cyclopropyl-3-(4,4- difluoropiperidin-1-yl)quinoxalin-5- yl)ethyl)amino)benzoic acid | 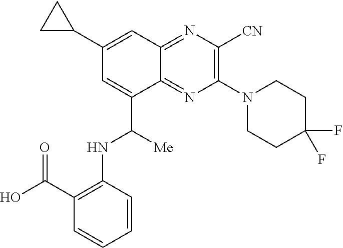 [SMILES: CC(Nc1ccccc1C(=O)O)c1cc(C2CC2)cc2nc(C#N)c(N3CCC(F)(F)CC3)nc12] |
| 2-((1-(2-cyano-3-(4,4-difluoropiperidin-1-yl)-7- methoxyquinoxalin-5-yl)ethyl)amino)benzoic acid | 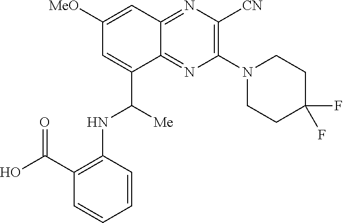 [SMILES: COc1cc(C(C)Nc2ccccc2C(=O)O)c2nc(N3CCC(F)(F)CC3)c(C#N)nc2c1] |
| 5-((1-(2-cyano-3-(4,4-difluoropiperidin-1-yl)-7- methylquinoxalin-5-yl)ethyl)amino)thiazole-4- carboxylic acid | 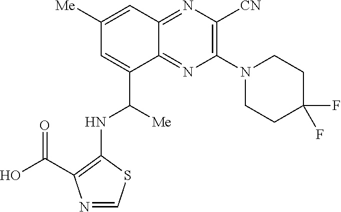 [SMILES: Cc1cc(C(C)Nc2scnc2C(=O)O)c2nc(N3CCC(F)(F)CC3)c(C#N)nc2c1] |
| 2-chloro-5-((1-(2-cyano-3-(4,4-difluoropiperidin- 1-yl)-7-methylquinoxalin-5-yl)ethyl)amino)thiazole-4- carboxylic acid | 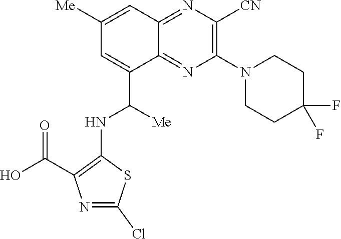 [SMILES: Cc1cc(C(C)Nc2sc(Cl)nc2C(=O)O)c2nc(N3CCC(F)(F)CC3)c(C#N)nc2c1] |
| 2-((1-(2-cyano-3-(4,4-difluoropiperidin-1-yl)-7- methylquinoxalin-5-yl)ethyl)amino)thiophene-3- carboxylic acid | 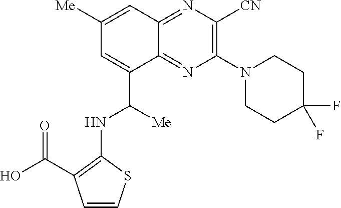 [SMILES: Cc1cc(C(C)Nc2sccc2C(=O)O)c2nc(N3CCC(F)(F)CC3)c(C#N)nc2c1] |
| 3-((1-(2-cyano-3-(4,4-difluoropiperidin-1-yl)-7- methylquinoxalin-5-yl)ethyl)amino)thiophene-2- carboxylic acid | 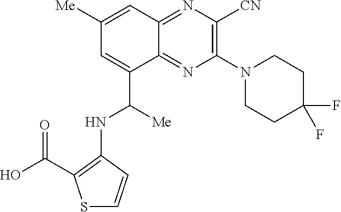 [SMILES: Cc1cc(C(C)Nc2ccsc2C(=O)O)c2nc(N3CCC(F)(F)CC3)c(C#N)nc2c1] |
| 6-chloro-3-((1-(2-cyano-7-methyl-3-(1- (trifluoromethyl)-3-azabicyclo[3.1.0]hexan-3- yl)quinoxalin-5-yl)ethyl)amino)picolinic acid | 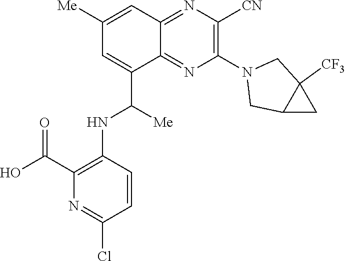 [SMILES: Cc1cc(C(C)Nc2ccc(Cl)nc2C(=O)O)c2nc(N3CC4CC4(C(F)(F)F)C3)c(C#N)nc2c1] |
| 6-chloro-3-((1-(2-cyano-3-cyclopropyl-7- methylquinoxalin-5-yl)ethyl)amino)picolinic acid | 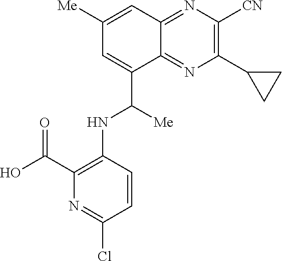 [SMILES: Cc1cc(C(C)Nc2ccc(Cl)nc2C(=O)O)c2nc(C3CC3)c(C#N)nc2c1] |
| 6-chloro-3-((1-(2-cyano-3-(3- (difluoromethyl)piperidin-1-yl)-7-methylquinoxalin-5- yl)ethyl)amino)picolinic acid | 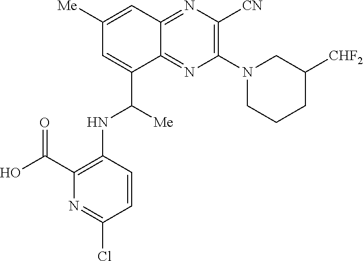 [SMILES: Cc1cc(C(C)Nc2ccc(Cl)nc2C(=O)O)c2nc(N3CCCC(C(F)F)C3)c(C#N)nc2c1] |
| 6-chloro-3-((1-(2-cyano-3-(1-(fluoromethyl)-3- azabicyclo[3.1.0]hexan-3-yl)-7-methylquinoxalin-5- yl)ethyl)amino)picolinic acid | 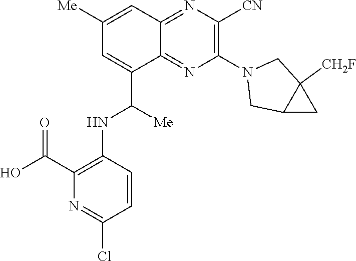 |
| 6-chloro-3-((1-(2-cyclopropyl-3-(4,4- difluoropiperidin-1-yl)-7-methylquinoxalin-5- yl)ethyl)amino)picolinic acid | 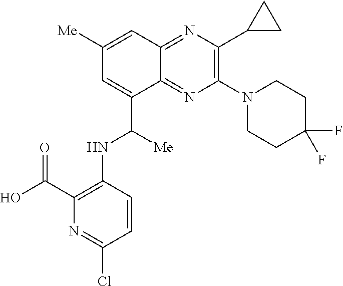 [SMILES: Cc1cc(C(C)Nc2ccc(Cl)nc2C(=O)O)c2nc(N3CCC(F)(F)CC3)c(C3CC3)nc2c1] |
| 6-chloro-3-((1-(3-(4,4-difluoropiperidin-1-yl)-7- methyl-2-(trifluoromethyl)quinoxalin-5- yl)ethyl)amino)picolinic acid | 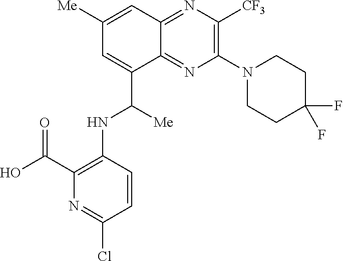 [SMILES: Cc1cc(C(C)Nc2ccc(Cl)nc2C(=O)O)c2nc(N3CCC(F)(F)CC3)c(C(F)(F)F)nc2c1] |
| 2-((1-(3-(4,4-difluoropiperidin-1-yl)-7-methyl-2- (trifluoromethyl)quinoxalin-5-yl)ethyl)amino)benzoic acid | 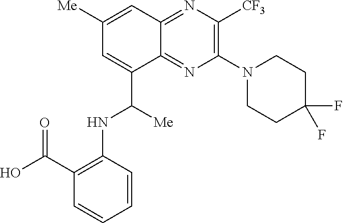 [SMILES: Cc1cc(C(C)Nc2ccccc2C(=O)O)c2nc(N3CCC(F)(F)CC3)c(C(F)(F)F)nc2c1] |
| 2-((1-(2-cyano-3-(4,4-difluoropiperidin-1-yl)-7- ethylquinoxalin-5-yl)ethyl)amino)benzoic acid | 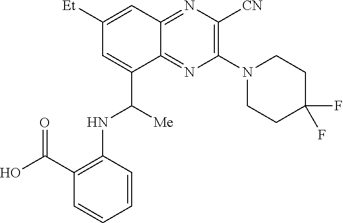 [SMILES: CCc1cc(C(C)Nc2ccccc2C(=O)O)c2nc(N3CCC(F)(F)CC3)c(C#N)nc2c1] |
| 6-chloro-3-((1-(2-chloro-3-(4,4-difluoropiperidin- 1-yl)-7-methylquinoxalin-5-yl)ethyl)amino)picolinic acid | 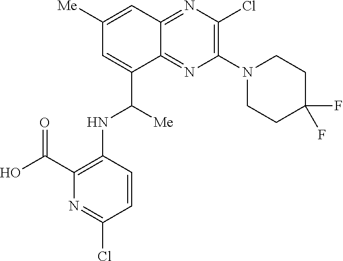 [SMILES: Cc1cc(C(C)Nc2ccc(Cl)nc2C(=O)O)c2nc(N3CCC(F)(F)CC3)c(Cl)nc2c1] |
| 2-((1-(2-chloro-3-(4,4-difluoropiperidin-1-yl)-7- methylquinoxalin-5-yl)ethyl)amino)benzoic acid | 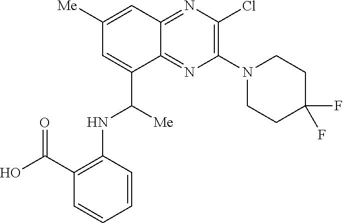 [SMILES: Cc1cc(C(C)Nc2ccccc2C(=O)O)c2nc(N3CCC(F)(F)CC3)c(Cl)nc2c1] |
| 3-((1-(3-(4,4-difluoropiperidin-1-yl)-2,7- dimethylquinoxalin-5-yl)ethyl)amino)-6- methylpicolinic acid | 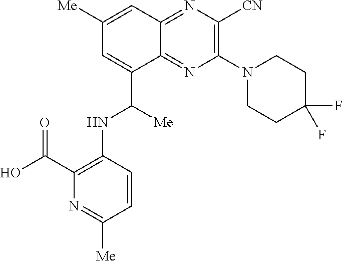 [SMILES: Cc1cc(C(C)Nc2ccc(C)nc2C(=O)O)c2nc(N3CCC(F)(F)CC3)c(C#N)nc2c1] |
| 2-((1-(2-cyano-3-(3,3-dimethylazetidin-1-yl)-7- methylquinoxalin-5-yl)ethyl)amino)benzoic acid | 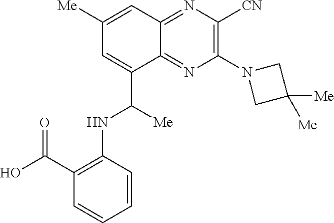 [SMILES: Cc1cc(C(C)Nc2ccccc2C(=O)O)c2nc(N3CC(C)(C)C3)c(C#N)nc2c1] |
| 6-chloro-3-((1-(2-cyano-3-(3,3-dimethylazetidin- 1-yl)-7-methylquinoxalin-5-yl)ethyl)amino)picolinic acid | 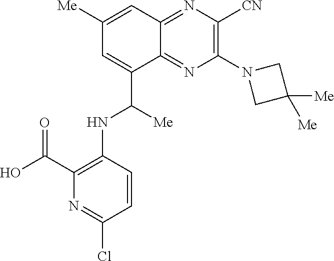 [SMILES: Cc1cc(C(C)Nc2ccc(Cl)nc2C(=O)O)c2nc(N3CC(C)(C)C3)c(C#N)nc2c1] |
| 2-((1-(2-cyano-7-methyl-3- morpholinoquinoxalin-5-yl)ethyl)amino)benzoic acid | 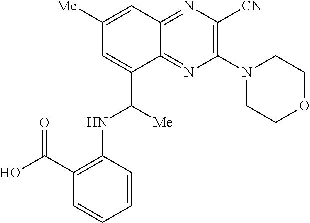 [SMILES: Cc1cc(C(C)Nc2ccccc2C(=O)O)c2nc(N3CCOCC3)c(C#N)nc2c1] |
| 6-chloro-3-((1-(2-cyano-7-methyl-3- morpholinoquinoxalin-5-yl)ethyl)amino)picolinic acid | 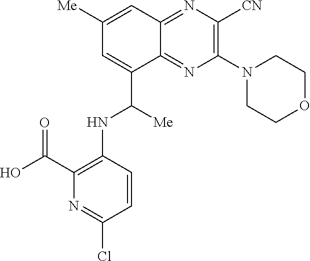 [SMILES: Cc1cc(C(C)Nc2ccc(Cl)nc2C(=O)O)c2nc(N3CCOCC3)c(C#N)nc2c1] |
| 2-((1-(2-cyano-7-methyl-3-(2-oxa-6- azaspiro[3.3]heptan-6-yl)quinoxalin-5- yl)ethyl)amino)benzoic acid | 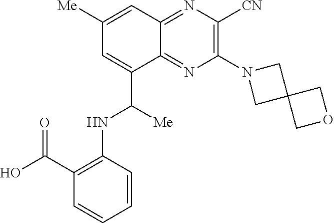 [SMILES: Cc1cc(C(C)Nc2ccccc2C(=O)O)c2nc(N3CC4(COC4)C3)c(C#N)nc2c1] |
| 6-chloro-3-((1-(2-cyano-7-methyl-3-(2-oxa-6- azaspiro[3.3]heptan-6-yl)quinoxalin-5- yl)ethyl)amino)picolinic acid | 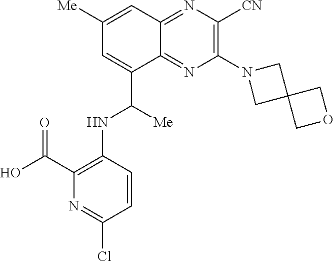 [SMILES: Cc1cc(C(C)Nc2ccc(Cl)nc2C(=O)O)c2nc(N3CC4(COC4)C3)c(C#N)nc2c1] |
| 2-((1-(2-cyano-3-(4,4-dimethylpiperidin-1-yl)-7- methylquinoxalin-5-yl)ethyl)amino)benzoic acid | 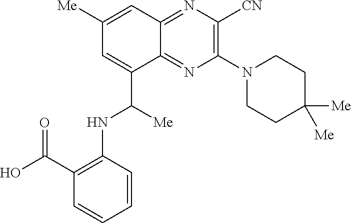 [SMILES: Cc1cc(C(C)Nc2ccccc2C(=O)O)c2nc(N3CCC(C)(C)CC3)c(C#N)nc2c1] |
| 6-chloro-3-((1-(2-cyano-3-(4,4- dimethylpiperidin-1-yl)-7-methylquinoxalin-5- yl)ethyl)amino)picolinic acid | 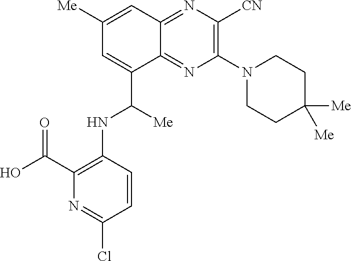 [SMILES: Cc1cc(C(C)Nc2ccc(Cl)nc2C(=O)O)c2nc(N3CCC(C)(C)CC3)c(C#N)nc2c1] |
| 2-((1-(2-cyano-3-(3-methoxy-3-methylazetidin- 1-yl)-7-methylquinoxalin-5-yl)ethyl)amino)benzoic acid | 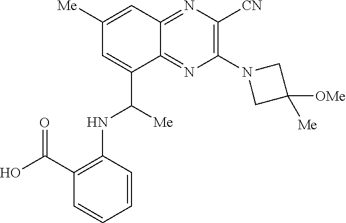 [SMILES: COC1(C)CN(c2nc3c(C(C)Nc4ccccc4C(=O)O)cc(C)cc3nc2C#N)C1] |
| 6-chloro-3-((1-(2-cyano-3-(3-methoxy-3- methylazetidin-1-yl)-7-methylquinoxalin-5- yl)ethyl)amino)picolinic acid | 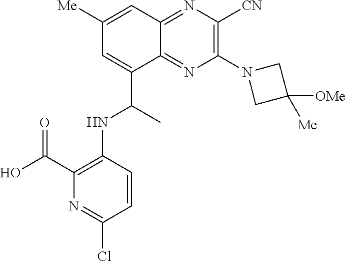 [SMILES: COC1(C)CN(c2nc3c(C(C)Nc4ccc(Cl)nc4C(=O)O)cc(C)cc3nc2C#N)C1] |
| 2-((1-(2-cyano-7-methyl-3-(4-methyl-4- (trifluoromethyl)piperidin-1-yl)quinoxalin-5- yl)ethyl)amino)benzoic acid | 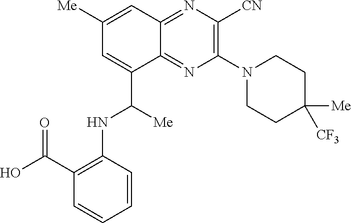 [SMILES: Cc1cc(C(C)Nc2ccccc2C(=O)O)c2nc(N3CCC(C)(C(F)(F)F)CC3)c(C#N)nc2c1] |
| 6-chloro-3-((1-(2-cyano-7-methyl-3-(4-methyl-4- (trifluoromethyl)piperidin-1-yl)quinoxalin-5- yl)ethyl)amino)picolinic acid | 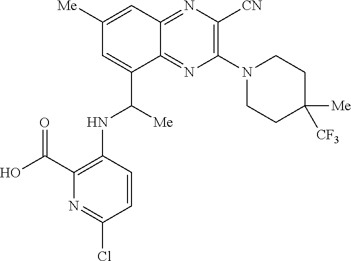 [SMILES: Cc1cc(C(C)Nc2ccc(Cl)nc2C(=O)O)c2nc(N3CCC(C)(C(F)(F)F)CC3)c(C#N)nc2c1] |
| 2-((1-(2-cyano-3-cyclopropyl-7- methylquinoxalin-5-yl)ethyl)amino)benzoic acid | 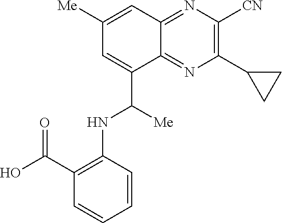 [SMILES: Cc1cc(C(C)Nc2ccccc2C(=O)O)c2nc(C3CC3)c(C#N)nc2c1] |
| 2-((1-(2-cyano-7-methyl-3-(pyrrolidin-1- yl)quinoxalin-5-yl)ethyl)amino)benzoic acid | 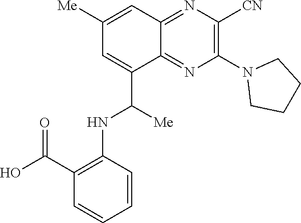 [SMILES: Cc1cc(C(C)Nc2ccccc2C(=O)O)c2nc(N3CCCC3)c(C#N)nc2c1] |
| 6-chloro-3-((1-(2-cyano-7-methyl-3-(pyrrolidin- 1-yl)quinoxalin-5-yl)ethyl)amino)picolinic acid | 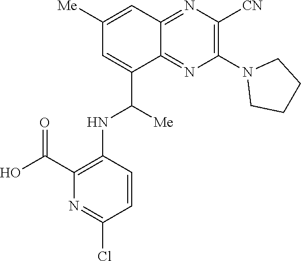 [SMILES: Cc1cc(C(C)Nc2ccc(Cl)nc2C(=O)O)c2nc(N3CCCC3)c(C#N)nc2c1] |
| 6-chloro-3-((1-(2-cyano-3-(3,3- difluoropyrrolidin-1-yl)-7-methylquinoxalin-5- yl)ethyl)amino)picolinic acid | 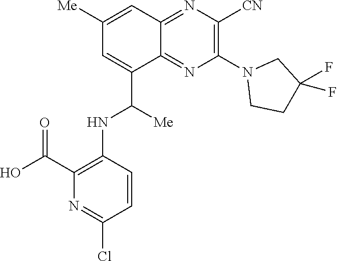 [SMILES: Cc1cc(C(C)Nc2ccc(Cl)nc2C(=O)O)c2nc(N3CCC(F)(F)C3)c(C#N)nc2c1] |
| 2-((1-(2-cyano-3-(3,3-difluoropyrrolidin-1-yl)-7- methylquinoxalin-5-yl)ethyl)amino)benzoic acid | 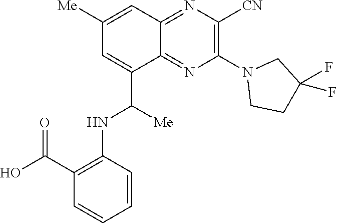 [SMILES: Cc1cc(C(C)Nc2ccccc2C(=O)O)c2nc(N3CCC(F)(F)C3)c(C#N)nc2c1] |
| 2-((1-(2-cyano-3-(3-methoxypyrrolidin-1-yl)-7- methylquinoxalin-5-yl)ethyl)amino)benzoic acid | 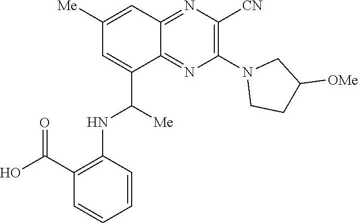 [SMILES: COC1CCN(c2nc3c(C(C)Nc4ccccc4C(=O)O)cc(C)cc3nc2C#N)C1] |
| 6-chloro-3-((1-(2-cyano-3-(3-methoxypyrrolidin- 1-yl)-7-methylquinoxalin-5-yl)ethyl)amino)picolinic acid | 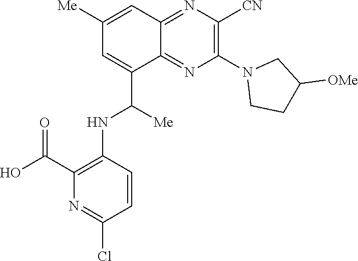 [SMILES: COC1CCN(c2nc3c(C(C)Nc4ccc(Cl)nc4C(=O)O)cc(C)cc3nc2C#N)C1] |
| 6-chloro-3-((1-(2-cyano-3-(((2,2-difluoro-1- methylcyclopropyl)methyl)amino)-7- methylquinoxalin-5-yl)ethyl)amino)picolinic acid | 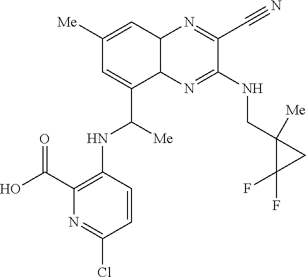 [SMILES: CC1=CC2N=C(C#N)C(NCC3(C)CC3(F)F)=NC2C(C(C)Nc2ccc(Cl)nc2C(=O)O)=C1] |
| 2-((1-(2-cyano-3-(((2,2-difluoro-1- methylcyclopropyl)methyl)amino)-7- methylquinoxalin-5-yl)ethyl)amino)benzoic acid | 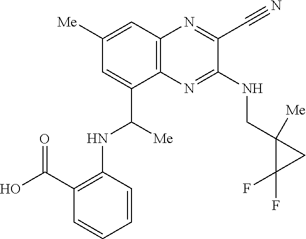 [SMILES: Cc1cc(C(C)Nc2ccccc2C(=O)O)c2nc(NCC3(C)CC3(F)F)c(C#N)nc2c1] |
| 2-((1-(2-cyano-3-((3,3- difluorocyclobutyl)amino)-7-methylquinoxalin-5- yl)ethyl)amino)benzoic acid | 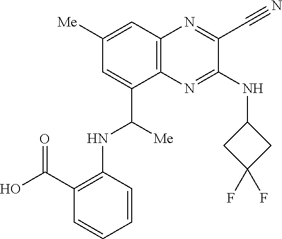 [SMILES: Cc1cc(C(C)Nc2ccccc2C(=O)O)c2nc(NC3CC(F)(F)C3)c(C#N)nc2c1] |
| 6-chloro-3-((1-(2-cyano-3-((3,3- difluorocyclobutyl)amino)-7-methylquinoxalin-5- yl)ethyl)amino)picolinic acid | 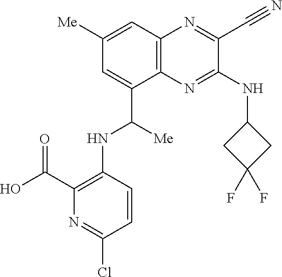 [SMILES: Cc1cc(C(C)Nc2ccc(Cl)nc2C(=O)O)c2nc(NC3CC(F)(F)C3)c(C#N)nc2c1] |
| 6-chloro-3-((1-(2-cyano-3-(4,4-difluoro-3- methylpiperidin-1-yl)-7-methylquinoxalin-5- yl)ethyl)amino)picolinic acid | 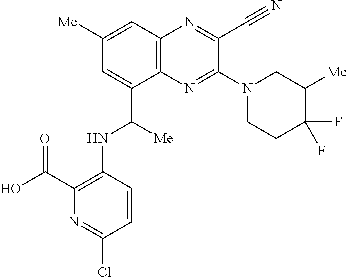 [SMILES: Cc1cc(C(C)Nc2ccc(Cl)nc2C(=O)O)c2nc(N3CCC(F)(F)C(C)C3)c(C#N)nc2c1] |
| 2-((1-(2-cyano-3-(4,4-difluoro-3- methylpiperidin-1-yl)-7-methylquinoxalin-5- yl)ethyl)amino)benzoic acid | 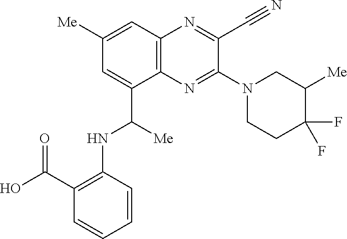 [SMILES: Cc1cc(C(C)Nc2ccccc2C(=O)O)c2nc(N3CCC(F)(F)C(C)C3)c(C#N)nc2c1] |
| 6-chloro-3-((1-(2-cyano-3-(3,3-difluoro-4- methylpyrrolidin-1-yl)-7-methylquinoxalin-5- yl)ethyl)amino)picolinic acid | 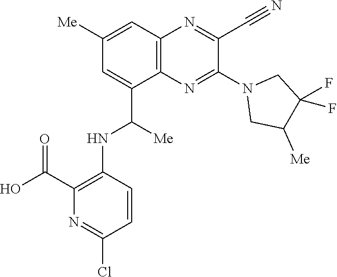 [SMILES: Cc1cc(C(C)Nc2ccc(Cl)nc2C(=O)O)c2nc(N3CC(C)C(F)(F)C3)c(C#N)nc2c1] |
| 2-((1-(2-cyano-3-(3,3-difluoro-4- methylpyrrolidin-1-yl)-7-methylquinoxalin-5- yl)ethyl)amino)benzoic acid | 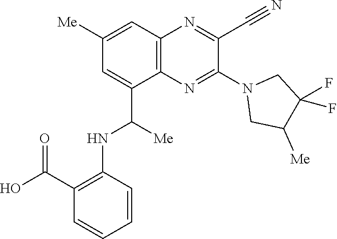 [SMILES: Cc1cc(C(C)Nc2ccccc2C(=O)O)c2nc(N3CC(C)C(F)(F)C3)c(C#N)nc2c1] |
| 6-chloro-3-((1-(2-cyano-7-methyl-3-(1,1,2,2- tetrafluoro-6-azaspiro[2.5]octan-6-yl)quinoxalin-5- yl)ethyl)amino)picolinic acid | 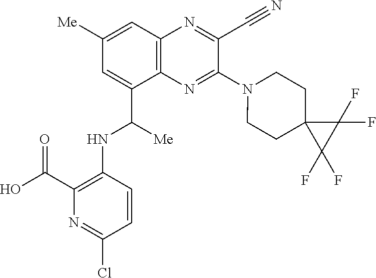 [SMILES: Cc1cc(C(C)Nc2ccc(Cl)nc2C(=O)O)c2nc(N3CCC4(CC3)C(F)(F)C4(F)F)c(C#N)nc2c1] |
| 6-chloro-3-((1-(2-cyano-3-(3,4- dihydroisoquinolin-2(1H)-yl)-7-methylquinoxalin-5- yl)ethyl)amino)picolinic acid | 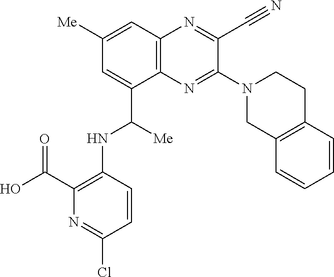 [SMILES: Cc1cc(C(C)Nc2ccc(Cl)nc2C(=O)O)c2nc(N3CCc4ccccc4C3)c(C#N)nc2c1] |
| 2-((1-(2-cyano-3-(3,4-dihydroisoquinolin-2(1H)- yl)-7-methylquinoxalin-5-yl)ethyl)amino)benzoic acid | 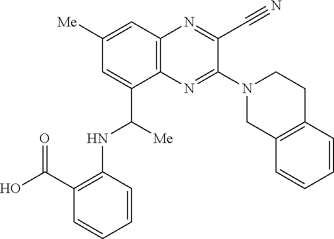 [SMILES: Cc1cc(C(C)Nc2ccccc2C(=O)O)c2nc(N3CCc4ccccc4C3)c(C#N)nc2c1] |
| 6-chloro-3-((1-(2-cyano-3-(6-cyano-3,4- dihydroisoquinolin-2(1H)-yl)-7-methylquinoxalin-5- yl)ethyl)amino)picolinic acid | 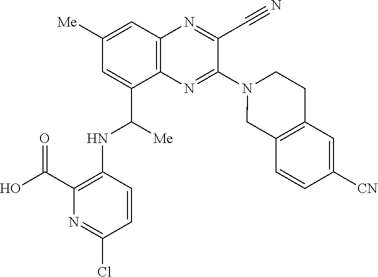 [SMILES: Cc1cc(C(C)Nc2ccc(Cl)nc2C(=O)O)c2nc(N3CCc4cc(C#N)ccc4C3)c(C#N)nc2c1] |
| 2-((1-(2-cyano-3-(6-cyano-3,4- dihydroisoquinolin-2(1H)-yl)-7-methylquinoxalin-5- yl)ethyl)amino)benzoic acid | 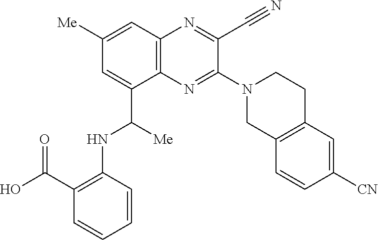 [SMILES: Cc1cc(C(C)Nc2ccccc2C(=O)O)c2nc(N3CCc4cc(C#N)ccc4C3)c(C#N)nc2c1] |
| 6-chloro-3-((1-(2-cyano-3-(6-fluoro-3,4- dihydroisoquinolin-2(1H)-yl)-7-methylquinoxalin-5- yl)ethyl)amino)picolinic acid | 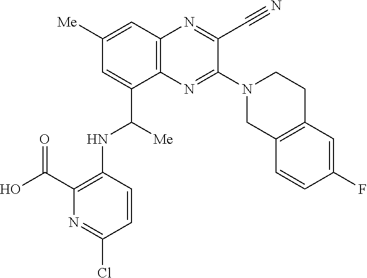 [SMILES: Cc1cc(C(C)Nc2ccc(Cl)nc2C(=O)O)c2nc(N3CCc4cc(F)ccc4C3)c(C#N)nc2c1] |
| 2-((1-(2-cyano-3-(6-fluoro-3,4- dihydroisoquinolin-2(1H)-yl)-7-methylquinoxalin-5- yl)ethyl)amino)benzoic acid | 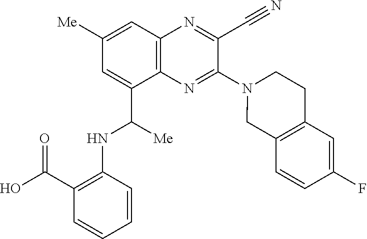 [SMILES: Cc1cc(C(C)Nc2ccccc2C(=O)O)c2nc(N3CCc4cc(F)ccc4C3)c(C#N)nc2c1] |
| 2-((1-(2-cyano-3-(3,4-dihydro-2,7-naphthyridin- 2(1H)-yl)-7-methylquinoxalin-5- yl)ethyl)amino)benzoic acid | 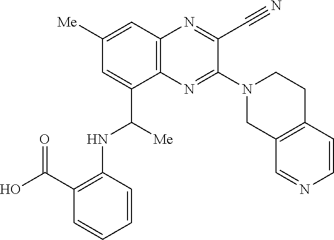 [SMILES: Cc1cc(C(C)Nc2ccccc2C(=O)O)c2nc(N3CCc4ccncc4C3)c(C#N)nc2c1] |
| 6-chloro-3-((1-(2-cyano-3-(4-(2- cyclopropylacetyl)piperazin-1-yl)-7-methylquinoxalin- 5-yl)ethyl)amino)picolinic acid | 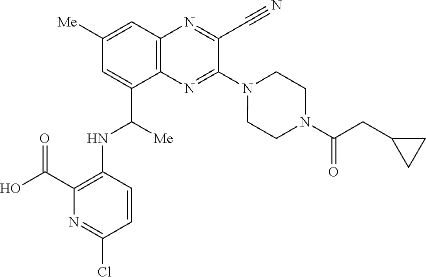 [SMILES: Cc1cc(C(C)Nc2ccc(Cl)nc2C(=O)O)c2nc(N3CCN(C(=O)CC4CC4)CC3)c(C#N)nc2c1] |
| 2-((1-(2-cyano-3-(4-(2- cyclopropylacetyl)piperazin-1-yl)-7-methylquinoxalin- 5-yl)ethyl)amino)benzoic acid | 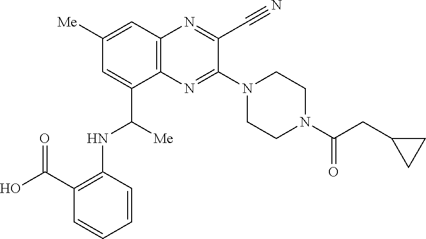 [SMILES: Cc1cc(C(C)Nc2ccccc2C(=O)O)c2nc(N3CCN(C(=O)CC4CC4)CC3)c(C#N)nc2c1] |
| 6-chloro-3-((1-(2-cyano-3-(4-(2-(2,2- difluorocyclopropyl)acetyl)piperazin-1-yl)-7- methylquinoxalin-5-yl)ethyl)amino)picolinic acid | 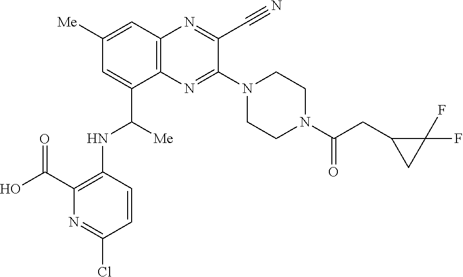 [SMILES: Cc1cc(C(C)Nc2ccc(Cl)nc2C(=O)O)c2nc(N3CCN(C(=O)CC4CC4(F)F)CC3)c(C#N)nc2c1] |
| 2-((1-(2-cyano-3-(4-(2-(2,2- difluorocyclopropyl)acetyl)piperazin-1-yl)-7- methylquinoxalin-5-yl)ethyl)amino)benzoic acid | 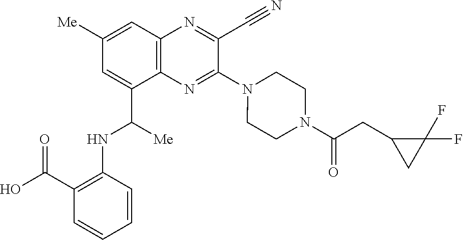 [SMILES: Cc1cc(C(C)Nc2ccccc2C(=O)O)c2nc(N3CCN(C(=O)CC4CC4(F)F)CC3)c(C#N)nc2c1] |
| 6-chloro-3-((1-(2-cyano-3-(1-hydroxy-6- azaspiro[3.5]nonan-6-yl)-7-methylquinoxalin-5- yl)ethyl)amino)picolinic acid | 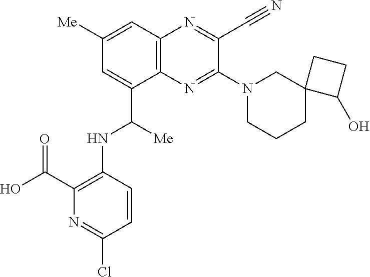 [SMILES: Cc1cc(C(C)Nc2ccc(Cl)nc2C(=O)O)c2nc(N3CCCC4(CCC4O)C3)c(C#N)nc2c1] |
| 2-((1-(2-cyano-3-(1-hydroxy-6- azaspiro[3.5]nonan-6-yl)-7-methylquinoxalin-5- yl)ethyl)amino)benzoic acid | 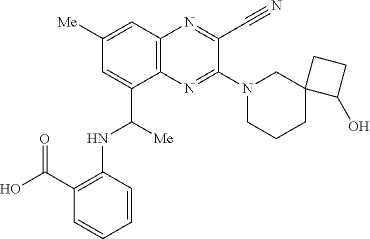 [SMILES: Cc1cc(C(C)Nc2ccccc2C(=O)O)c2nc(N3CCCC4(CCC4O)C3)c(C#N)nc2c1] |
| 6-chloro-3-((1-(2-cyano-7-methyl-3-(3-(1- methyl-1H-pyrazol-5-yl)-8-azabicyclo[3.2.1]octan-8- yl)quinoxalin-5-yl)ethyl)amino)picolinic acid | 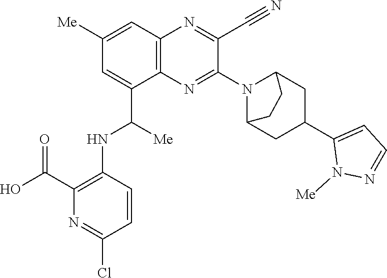 [SMILES: Cc1cc(C(C)Nc2ccc(Cl)nc2C(=O)O)c2nc(N3C4CCC3CC(c3ccnn3C)C4)c(C#N)nc2c1] |
| 2-((1-(2-cyano-7-methyl-3-(3-(1-methyl-1H- pyrazol-5-yl)-8-azabicyclo[3.2.1]octan-8- yl)quinoxalin-5-yl)ethyl)amino)benzoic acid | 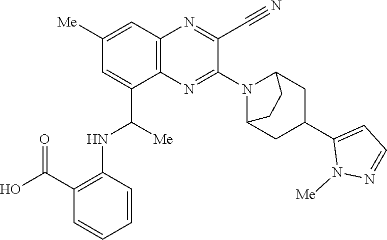 [SMILES: Cc1cc(C(C)Nc2ccccc2C(=O)O)c2nc(N3C4CCC3CC(c3ccnn3C)C4)c(C#N)nc2c1] |
| 2-((1-(2-cyano-7-methyl-3-(3-(1-methyl-1H- pyrazol-3-yl)-8-azabicyclo[3.2.1]octan-8- yl)quinoxalin-5-yl)ethyl)amino)benzoic acid | 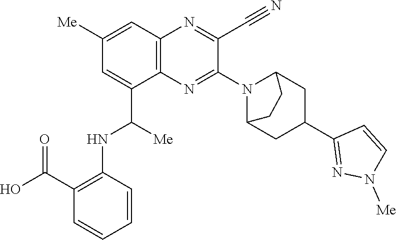 [SMILES: Cc1cc(C(C)Nc2ccccc2C(=O)O)c2nc(N3C4CCC3CC(c3ccn(C)n3)C4)c(C#N)nc2c1] |
| 2-((1-(2-cyano-7-methyl-3-(3-(1-methyl-1H- pyrazol-4-yl)-8-azabicyclo[3.2.1]octan-8- yl)quinoxalin-5-yl)ethyl)amino)benzoic acid | 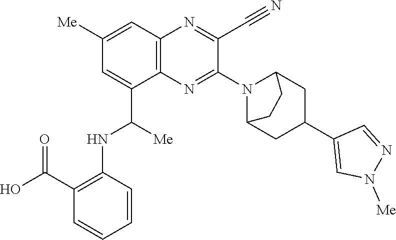 [SMILES: Cc1cc(C(C)Nc2ccccc2C(=O)O)c2nc(N3C4CCC3CC(c3cnn(C)c3)C4)c(C#N)nc2c1] |
| 6-chloro-3-((1-(2-cyano-7-methyl-3-(3-(1- methyl-1H-pyrazol-3-yl)-8-azabicyclo[3.2.1]octan-8- yl)quinoxalin-5-yl)ethyl)amino)picolinic acid | 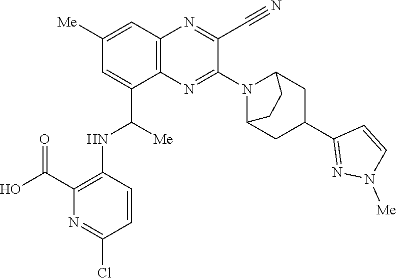 [SMILES: Cc1cc(C(C)Nc2ccc(Cl)nc2C(=O)O)c2nc(N3C4CCC3CC(c3ccn(C)n3)C4)c(C#N)nc2c1] |
| 6-chloro-3-((1-(2-cyano-7-methyl-3-(3-(1- methyl-1H-pyrazol-4-yl)-8-azabicyclo[3.2.1]octan-8- yl)quinoxalin-5-yl)ethyl)amino)picolinic acid | 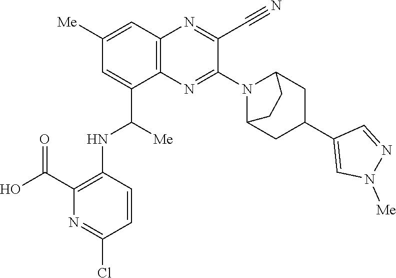 [SMILES: Cc1cc(C(C)Nc2ccc(Cl)nc2C(=O)O)c2nc(N3C4CCC3CC(c3cnn(C)c3)C4)c(C#N)nc2c1] |
| 6-chloro-3-((1-(2-cyano-7-methyl-3-(2-oxo-[1,4′- bipiperidin]-1′-yl)quinoxalin-5-yl)ethyl) amino)picolinic acid | 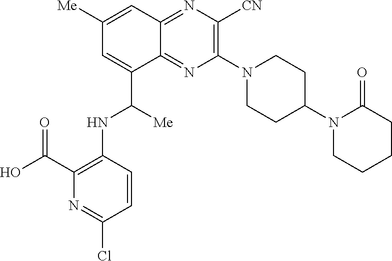 [SMILES: Cc1cc(C(C)Nc2ccc(Cl)nc2C(=O)O)c2nc(N3CCC(N4CCCCC4=O)CC3)c(C#N)nc2c1] |
| 6-chloro-3-(((1R)-1-(2-cyano-7-methyl-3-(3-(2- oxopiperidin-1-yl)-8-azabicyclo[3.2.1]octan-8- yl)quinoxalin-5-yl)ethyl) amino)picolinic acid | 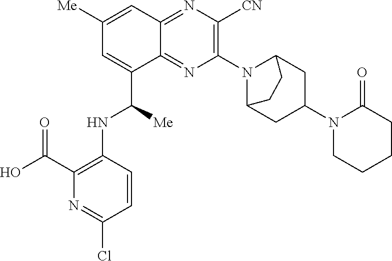 [SMILES: Cc1cc([C@@H](C)Nc2ccc(Cl)nc2C(=O)O)c2nc(N3C4CCC3CC(N3CCCCC3=O)C4)c(C#N)nc2c1] |
| 2-((1-(2-cyano-7-methyl-3-(2-oxo-[1,4′- bipiperidin]-1′-yl)quinoxalin-5-yl)ethyl)amino)benzoic acid | 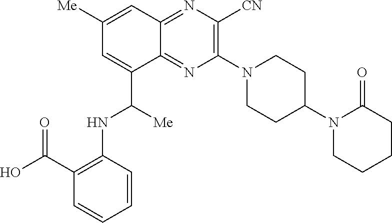 [SMILES: Cc1cc(C(C)Nc2ccccc2C(=O)O)c2nc(N3CCC(N4CCCCC4=O)CC3)c(C#N)nc2c1] |
| 6-chloro-3-((1-(2-cyano-3-(4- (ethoxymethyl)piperidin-1-yl)-7-methylquinoxalin-5- yl)ethyl)amino)picolinic acid | 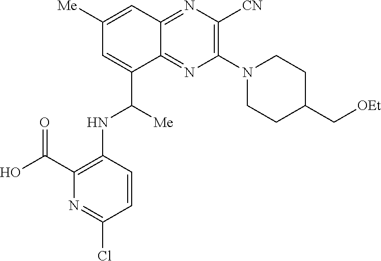 [SMILES: CCOCC1CCN(c2nc3c(C(C)Nc4ccc(Cl)nc4C(=O)O)cc(C)cc3nc2C#N)CC1] |
| 2-((1-(2-cyano-3-(4-(ethoxymethyl)piperidin-1- yl)-7-methylquinoxalin-5-yl)ethyl)amino)benzoic acid | 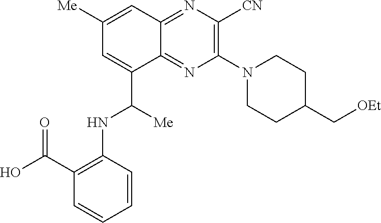 [SMILES: CCOCC1CCN(c2nc3c(C(C)Nc4ccccc4C(=O)O)cc(C)cc3nc2C#N)CC1] |
| or a pharmaceutically acceptable salt thereof. | |

In one embodiment, the compound of Formula (I) is selected from:

| Name | Structure |
|---|---|
| (R)-2-((1-(2-cyano-3-(4,4-difluoropiperidin-1-yl)-7- methylquinoxalin-5-yl)ethyl)amino)benzoic acid | 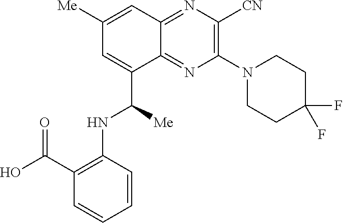 [SMILES: Cc1cc([C@@H](C)Nc2ccccc2C(=O)O)c2nc(N3CCC(F)(F)CC3)c(C#N)nc2c1] |
| (S)-2-((1-(2-cyano-3-(4,4-difluoropiperidin-1-yl)-7- methylquinoxalin-5-yl)ethyl)amino)benzoic acid | 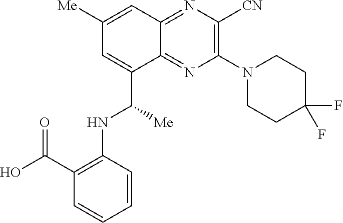 [SMILES: Cc1cc([C@H](C)Nc2ccccc2C(=O)O)c2nc(N3CCC(F)(F)CC3)c(C#N)nc2c1] |
| (R)-6-chloro-3-((1-(2-cyano-3-(4,4-difluoropiperidin- 1-yl)-7-methylquinoxalin-5-yl)ethyl)amino)picolinic acid | 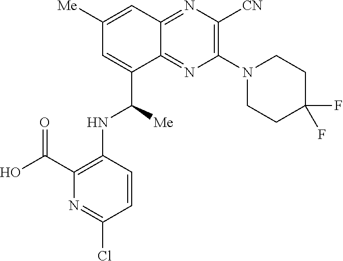 [SMILES: Cc1cc([C@@H](C)Nc2ccc(Cl)nc2C(=O)O)c2nc(N3CCC(F)(F)CC3)c(C#N)nc2c1] |
| (R)-2-((1-(2-cyano-3-(isoindolin-2-yl)-7- methylquinoxalin-5-yl)ethyl)amino)benzoic acid | 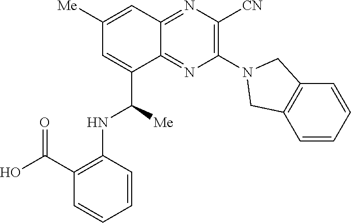 [SMILES: Cc1cc([C@@H](C)Nc2ccccc2C(=O)O)c2nc(N3Cc4ccccc4C3)c(C#N)nc2c1] |
| 6-chloro-3-(((1R)-1-(2-cyano-3-(6,6-difluoro-3- azabicyclo[3.1.1]heptan-3-yl)-7-methylquinoxalin-5- yl)ethyl)amino)picolinic acid | 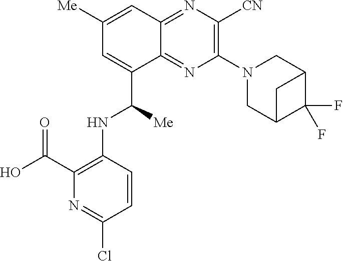 [SMILES: Cc1cc([C@@H](C)Nc2ccc(Cl)nc2C(=O)O)c2nc(N3CC4CC(C3)C4(F)F)c(C#N)nc2c1] |
| 6-chloro-3-(((R)-1-(2-cyano-3-((1S,4S)-5,5-difluoro- 2-azabicyclo[2.2.1]heptan-2-yl)-7-methylquinoxalin- 5-yl)ethyl)amino)picolinic acid | 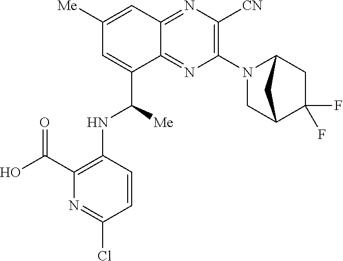 [SMILES: Cc1cc([C@@H](C)Nc2ccc(Cl)nc2C(=O)O)c2nc(N3C[C@@H]4C[C@H]3CC4(F)F)c(C#N)nc2c1] |
| 6-chloro-3-(((R)-1-(2-cyano-3-((1R,4R)-5,5-difluoro- 2-azabicyclo[2.2.1]heptan-2-yl)-7-methylquinoxalin- 5-yl)ethyl)amino)picolinic acid | 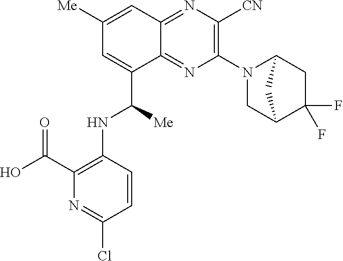 [SMILES: Cc1cc([C@@H](C)Nc2ccc(Cl)nc2C(=O)O)c2nc(N3C[C@H]4C[C@@H]3CC4(F)F)c(C#N)nc2c1] |
| 6-chloro-3-(((R)-1-(2-cyano-3-((1R,4S)-6,6-difluoro- 2-azabicyclo[2.2.1]heptan-2-yl)-7-methylquinoxalin- 5-yl)ethyl)amino)picolinic acid | 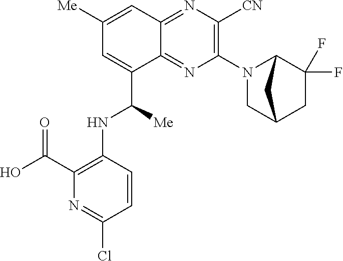 [SMILES: Cc1cc([C@@H](C)Nc2ccc(Cl)nc2C(=O)O)c2nc(N3C[C@@H]4C[C@H]3C(F)(F)C4)c(C#N)nc2c1] |
| 6-chloro-3-(((R)-1-(2-cyano-3-((1S,4R)-6,6-difluoro- 2-azabicyclo[2.2.1]heptan-2-yl)-7-methylquinoxalin- 5-yl)ethyl)amino)picolinic acid | 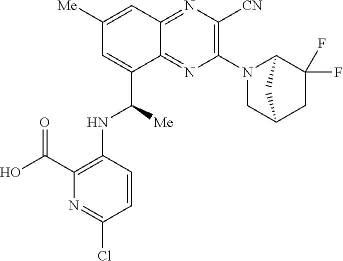 [SMILES: Cc1cc([C@@H](C)Nc2ccc(Cl)nc2C(=O)O)c2nc(N3C[C@H]4C[C@@H]3C(F)(F)C4)c(C#N)nc2c1] |
| (R)-6-chloro-3-((1-(2-cyano-3-(3,3-difluoropiperidin- 1-yl)-7-methylquinoxalin-5-yl)ethyl)amino)picolinic acid | 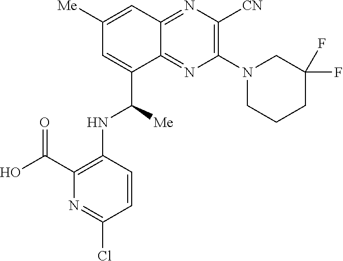 [SMILES: Cc1cc([C@@H](C)Nc2ccc(Cl)nc2C(=O)O)c2nc(N3CCCC(F)(F)C3)c(C#N)nc2c1] |
| 6-chloro-3-(((R)-1-(2-cyano-3-((((S)-2,2- difluorocyclopropyl)methyl)amino)-7- methylquinoxalin-5-yl)ethyl)amino)picolinic acid | 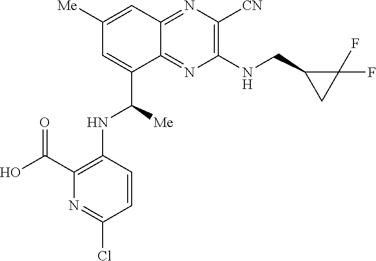 [SMILES: Cc1cc([C@@H](C)Nc2ccc(Cl)nc2C(=O)O)c2nc(NC[C@@H]3CC3(F)F)c(C#N)nc2c1] |
| 6-chloro-3-(((R)-1-(2-cyano-3-((((R)-2,2- difluorocyclopropyl)methyl)amino)-7- methylquinoxalin-5-yl)ethyl)amino)picolinic acid | 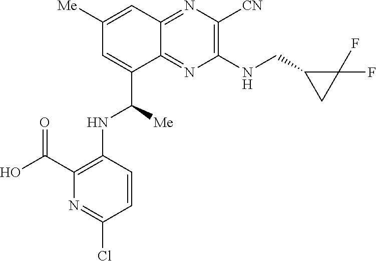 [SMILES: Cc1cc([C@@H](C)Nc2ccc(Cl)nc2C(=O)O)c2nc(NC[C@H]3CC3(F)F)c(C#N)nc2c1] |
| (R)-6-chloro-3-((1-(2-cyano-7-methyl-3-(((1- (trifluoromethyl)cyclopropyl)methyl)amino)quinoxalin- 5-yl)ethyl)amino)picolinic acid | 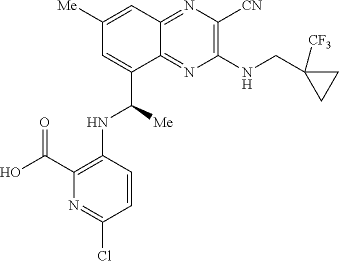 [SMILES: Cc1cc([C@@H](C)Nc2ccc(Cl)nc2C(=O)O)c2nc(NCC3(C(F)(F)F)CC3)c(C#N)nc2c1] |
| (R)-6-chloro-3-((1-(2-cyano-3-(1,1-difluoro-5- azaspiro[2.3]hexan-5-yl)-7-methylquinoxalin-5- yl)ethyl)amino)picolinic acid | 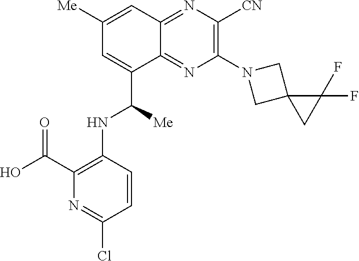 [SMILES: Cc1cc([C@@H](C)Nc2ccc(Cl)nc2C(=O)O)c2nc(N3CC4(C3)CC4(F)F)c(C#N)nc2c1] |
| (R)-3-((1-(2-cyano-3-(4,4-difluoropiperidin-1-yl)-7- methylquinoxalin-5-yl)ethyl)amino)-6-methylpicolinic acid | 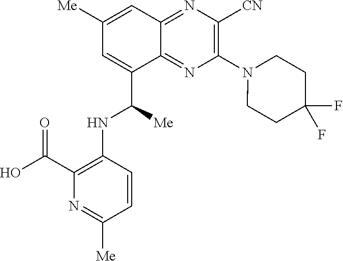 [SMILES: Cc1cc([C@@H](C)Nc2ccc(C)nc2C(=O)O)c2nc(N3CCC(F)(F)CC3)c(C#N)nc2c1] |
| (R)-2-((1-(2-cyano-3-((cyclopropylmethyl)amino)-7- methylquinoxalin-5-yl)ethyl)amino)benzoic acid | 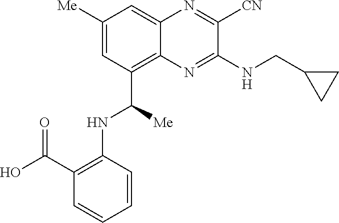 [SMILES: Cc1cc([C@@H](C)Nc2ccccc2C(=O)O)c2nc(NCC3CC3)c(C#N)nc2c1] |
| 6-chloro-3-(((1R)-1-(2-cyano-7-methyl-3-(3- (trifluoromethyl)piperidin-1-yl)quinoxalin-5- yl)ethyl)amino) picolinic acid | 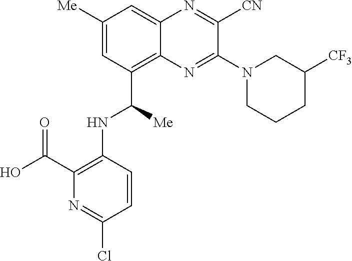 [SMILES: Cc1cc([C@@H](C)Nc2ccc(Cl)nc2C(=O)O)c2nc(N3CCCC(C(F)(F)F)C3)c(C#N)nc2c1] |
| (R)-6-chloro-3-((1-(2-cyano-3-(4,4-difluoropiperidin- 1-yl)-7-(trifluoromethyl)quinoxalin-5- yl)ethyl)amino)picolinic acid | 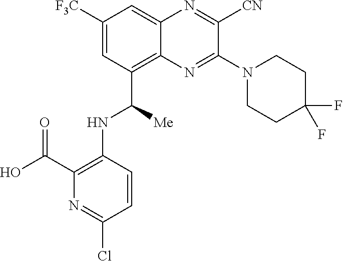 [SMILES: C[C@@H](Nc1ccc(Cl)nc1C(=O)O)c1cc(C(F)(F)F)cc2nc(C#N)c(N3CCC(F)(F)CC3)nc12] |
| 6-chloro-3-(((1R)-1-(2-cyano-3-(6,6-difluoro-3- azabicyclo[3.1.0]hexan-3-yl)-7-methylquinoxalin-5- yl)ethyl)amino)picolinic acid | 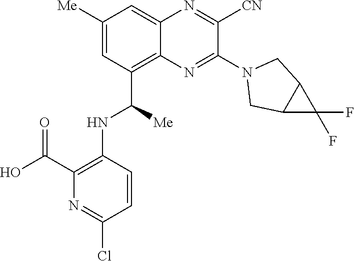 [SMILES: Cc1cc([C@@H](C)Nc2ccc(Cl)nc2C(=O)O)c2nc(N3CC4C(C3)C4(F)F)c(C#N)nc2c1] |
| (R)-6-chloro-3-((1-(2-cyano-3-(4,4-difluoro-6- azaspiro[2.5]octan-6-yl)-7-methylquinoxalin-5- yl)ethyl)amino)picolinic acid | 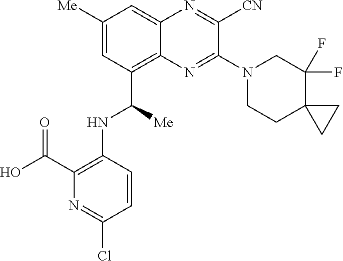 [SMILES: Cc1cc([C@@H](C)Nc2ccc(Cl)nc2C(=O)O)c2nc(N3CCC4(CC4)C(F)(F)C3)c(C#N)nc2c1] |
| (R)-6-chloro-3-((1-(2-cyano-3-(((1- fluorocyclopropyl)methyl)amino)-7-methylquinoxalin- 5-yl)ethyl)amino)picolinic acid | 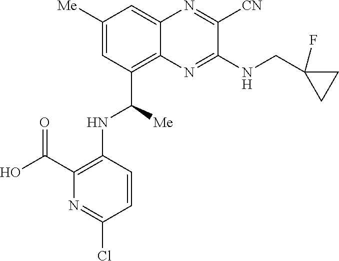 [SMILES: Cc1cc([C@@H](C)Nc2ccc(Cl)nc2C(=O)O)c2nc(NCC3(F)CC3)c(C#N)nc2c1] |
| (R)-6-chloro-3-((1-(2-cyano-3-(5-fluoroisoindolin-2- yl)-7-methylquinoxalin-5-yl)ethyl)amino)picolinic acid | 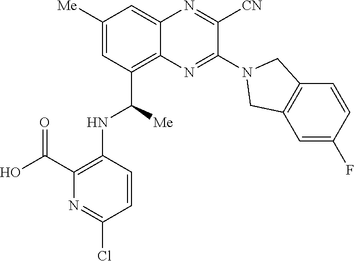 [SMILES: Cc1cc([C@@H](C)Nc2ccc(Cl)nc2C(=O)O)c2nc(N3Cc4ccc(F)cc4C3)c(C#N)nc2c1] |
| (R)-6-chloro-3-((1-(2-cyano-3- ((cyclopropylmethyl)amino)-7-methylquinoxalin-5- yl)ethyl)amino)picolinic acid | 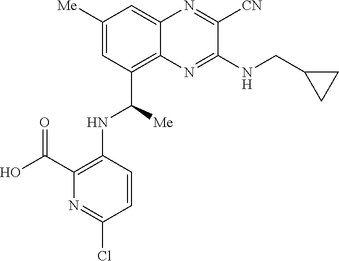 [SMILES: Cc1cc([C@@H](C)Nc2ccc(Cl)nc2C(=O)O)c2nc(NCC3CC3)c(C#N)nc2c1] |
| (R)-6-chloro-3-((1-(2-cyano-3-(4,4-difluoropiperidin- 1-yl)-7-fluoroquinoxalin-5-yl)ethyl)amino)picolinic acid | 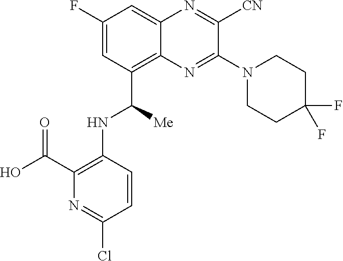 [SMILES: C[C@@H](Nc1ccc(Cl)nc1C(=O)O)c1cc(F)cc2nc(C#N)c(N3CCC(F)(F)CC3)nc12] |
| (R)-2-((1-(2-cyano-3-(4,4-difluoropiperidin-1-yl)-7- fluoroquinoxalin-5-yl)ethyl)amino)benzoic acid | 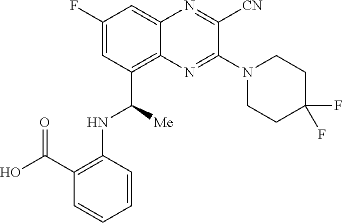 [SMILES: C[C@@H](Nc1ccccc1C(=O)O)c1cc(F)cc2nc(C#N)c(N3CCC(F)(F)CC3)nc12] |
| (S)-2-((1-(2-cyano-3-(4,4-difluoropiperidin-1-yl)-7- fluoroquinoxalin-5-yl)ethyl)amino)benzoic acid | 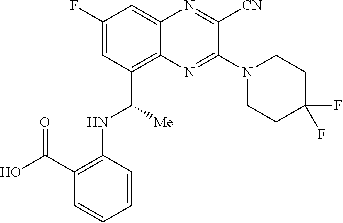 [SMILES: C[C@H](Nc1ccccc1C(=O)O)c1cc(F)cc2nc(C#N)c(N3CCC(F)(F)CC3)nc12] |
| (S)-2-((1-(2,7-dicyano-3-(4,4-difluoropiperidin-1- yl)quinoxalin-5-yl)ethyl)amino)benzoic acid | 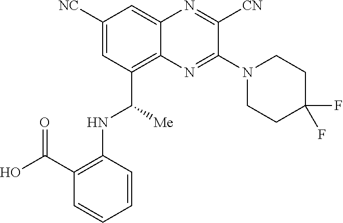 [SMILES: C[C@H](Nc1ccccc1C(=O)O)c1cc(C#N)cc2nc(C#N)c(N3CCC(F)(F)CC3)nc12] |
| (R)-2-((1-(2,7-dicyano-3-(4,4-difluoropiperidin-1- yl)quinoxalin-5-yl)ethyl)amino)benzoic acid | 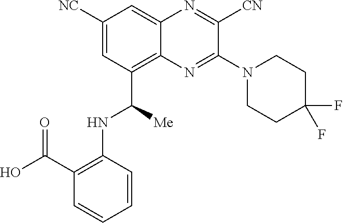 [SMILES: C[C@@H](Nc1ccccc1C(=O)O)c1cc(C#N)cc2nc(C#N)c(N3CCC(F)(F)CC3)nc12] |
| (S)-2-((1-(7-chloro-2-cyano-3-(4,4-difluoropiperidin- 1-yl)quinoxalin-5-yl)ethyl)amino)benzoic acid | 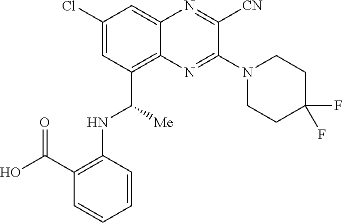 [SMILES: C[C@H](Nc1ccccc1C(=O)O)c1cc(Cl)cc2nc(C#N)c(N3CCC(F)(F)CC3)nc12] |
| (R)-2-((1-(7-chloro-2-cyano-3-(4,4-difluoropiperidin- 1-yl)quinoxalin-5-yl)ethyl)amino)benzoic acid | 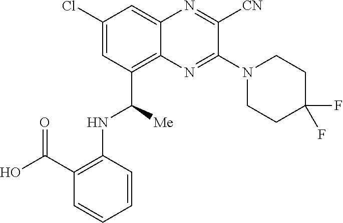 [SMILES: C[C@@H](Nc1ccccc1C(=O)O)c1cc(Cl)cc2nc(C#N)c(N3CCC(F)(F)CC3)nc12] |
| (R)-6-chloro-3-((1-(2-cyano-7-cyclopropyl-3-(4,4- difluoropiperidin-1-yl)quinoxalin-5- yl)ethyl)amino)picolinic acid | 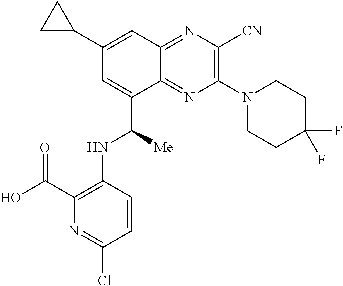 [SMILES: C[C@@H](Nc1ccc(Cl)nc1C(=O)O)c1cc(C2CC2)cc2nc(C#N)c(N3CCC(F)(F)CC3)nc12] |
| (R)-6-chloro-3-((1-(7-chloro-2-cyano-3-(4,4- difluoropiperidin-1-yl)quinoxalin-5- yl)ethyl)amino)picolinic acid | 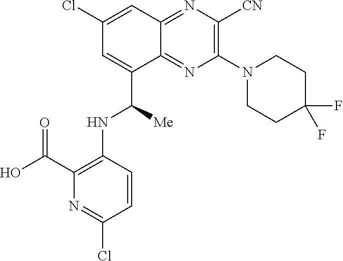 [SMILES: C[C@@H](Nc1ccc(Cl)nc1C(=O)O)c1cc(Cl)cc2nc(C#N)c(N3CCC(F)(F)CC3)nc12] |
| (R)-6-chloro-3-((1-(3-(4,4-difluoropiperidin-1-yl)-2,7- dimethylquinoxalin-5-yl)ethyl)amino)picolinic acid | 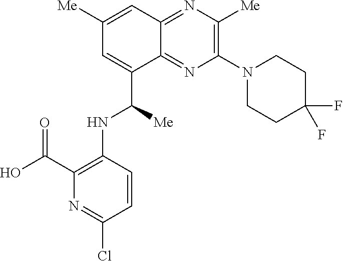 [SMILES: Cc1cc([C@@H](C)Nc2ccc(Cl)nc2C(=O)O)c2nc(N3CCC(F)(F)CC3)c(C)nc2c1] |
| (R)-2-((1-(3-(4,4-difluoropiperidin-1-yl)-2,7- dimethylquinoxalin-5-yl)ethyl)amino)benzoic acid | 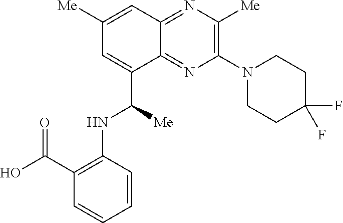 [SMILES: Cc1cc([C@@H](C)Nc2ccccc2C(=O)O)c2nc(N3CCC(F)(F)CC3)c(C)nc2c1] |
| (R)-2-((1-(2-cyano-7-cyclopropyl-3-(4,4- difluoropiperidin-1-yl)quinoxalin-5- yl)ethyl)amino)benzoic acid | 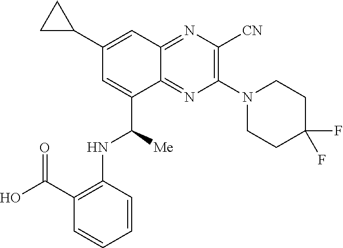 [SMILES: C[C@@H](Nc1ccccc1C(=O)O)c1cc(C2CC2)cc2nc(C#N)c(N3CCC(F)(F)CC3)nc12] |
| (R)-2-((1-(2-cyano-3-(4,4-difluoropiperidin-1-yl)-7- methoxyquinoxalin-5-yl)ethyl)amino) benzoic acid | 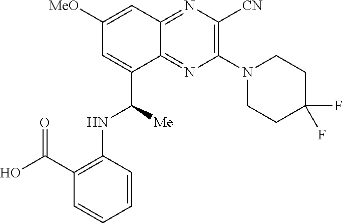 [SMILES: COc1cc([C@@H](C)Nc2ccccc2C(=O)O)c2nc(N3CCC(F)(F)CC3)c(C#N)nc2c1] |
| (R)-5-((1-(2-cyano-3-(4,4-difluoropiperidin-1-yl)-7- methylquinoxalin-5-yl)ethyl)amino)thiazole-4- carboxylic acid | 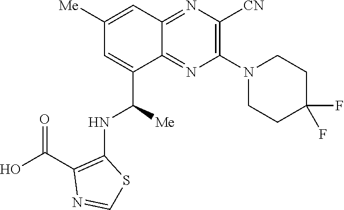 [SMILES: Cc1cc([C@@H](C)Nc2scnc2C(=O)O)c2nc(N3CCC(F)(F)CC3)c(C#N)nc2c1] |
| (R)-2-chloro-5-((1-(2-cyano-3-(4,4-difluoropiperidin- 1-yl)-7-methylquinoxalin-5-yl)ethyl)amino)thiazole-4- carboxylic acid | 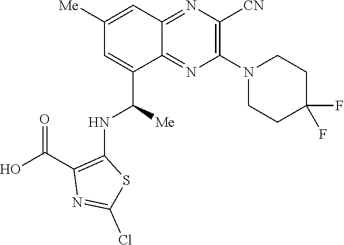 [SMILES: Cc1cc([C@@H](C)Nc2sc(Cl)nc2C(=O)O)c2nc(N3CCC(F)(F)CC3)c(C#N)nc2c1] |
| (R)-2-((1-(2-cyano-3-(4,4-difluoropiperidin-1-yl)-7- methylquinoxalin-5-yl)ethyl)amino)thiophene-3- carboxylic acid | 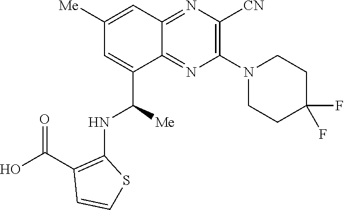 [SMILES: Cc1cc([C@@H](C)Nc2sccc2C(=O)O)c2nc(N3CCC(F)(F)CC3)c(C#N)nc2c1] |
| (R)-3-((1-(2-cyano-3-(4,4-difluoropiperidin-1-yl)-7- methylquinoxalin-5-yl)ethyl)amino)thiophene-2- carboxylic acid | 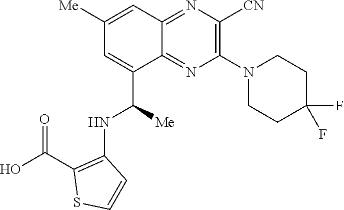 [SMILES: Cc1cc([C@@H](C)Nc2ccsc2C(=O)O)c2nc(N3CCC(F)(F)CC3)c(C#N)nc2c1] |
| 6-chloro-3-(((1R)-1-(2-cyano-7-methyl-3-(1- (trifluoromethyl)-3-azabicyclo[3.1.0]hexan-3- yl)quinoxalin-5-yl)ethyl)amino)picolinic acid | 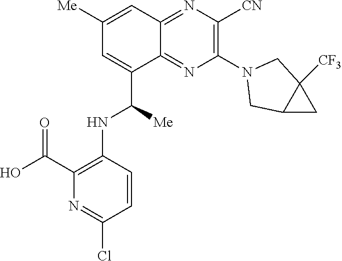 [SMILES: Cc1cc([C@@H](C)Nc2ccc(Cl)nc2C(=O)O)c2nc(N3CC4CC4(C(F)(F)F)C3)c(C#N)nc2c1] |
| (R)-6-chloro-3-((1-(2-cyano-3-cyclopropyl-7- methylquinoxalin-5-yl)ethyl)amino)picolinic acid | 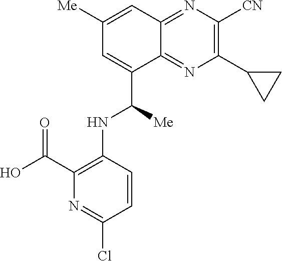 [SMILES: Cc1cc([C@@H](C)Nc2ccc(Cl)nc2C(=O)O)c2nc(C3CC3)c(C#N)nc2c1] |
| 6-chloro-3-(((R)-1-(2-cyano-7-methyl-3-((R)-3- (trifluoromethyl)piperidin-1-yl)quinoxalin-5- yl)ethyl)amino)picolinic acid | 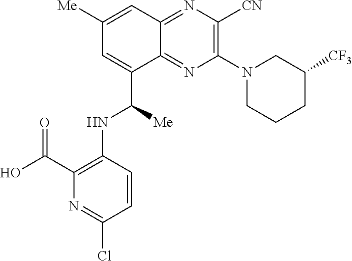 [SMILES: Cc1cc([C@@H](C)Nc2ccc(Cl)nc2C(=O)O)c2nc(N3CCC[C@@H](C(F)(F)F)C3)c(C#N)nc2c1] |
| 6-chloro-3-(((R)-1-(2-cyano-7-methyl-3-((S)-3- (trifluoromethyl)piperidin-1-yl)quinoxalin-5- yl)ethyl)amino)picolinic acid | 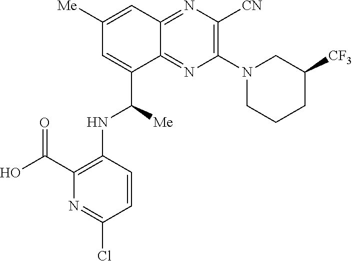 [SMILES: Cc1cc([C@@H](C)Nc2ccc(Cl)nc2C(=O)O)c2nc(N3CCC[C@H](C(F)(F)F)C3)c(C#N)nc2c1] |
| 6-chloro-3-(((1R)-1-(2-cyano-7-methyl-3-((1S)-1- (trifluoromethyl)-3-azabicyclo[3.1.0]hexan-3- yl)quinoxalin-5-yl)ethyl)amino)picolinic acid | 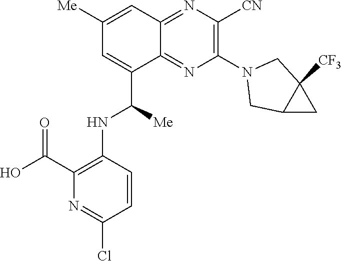 [SMILES: Cc1cc([C@@H](C)Nc2ccc(Cl)nc2C(=O)O)c2nc(N3CC4C[C@@]4(C(F)(F)F)C3)c(C#N)nc2c1] |
| 6-chloro-3-(((1R)-1-(2-cyano-7-methyl-3-((1R)-1- (trifluoromethyl)-3-azabicyclo[3.1.0]hexan-3- yl)quinoxalin-5-yl)ethyl)amino)picolinic acid | 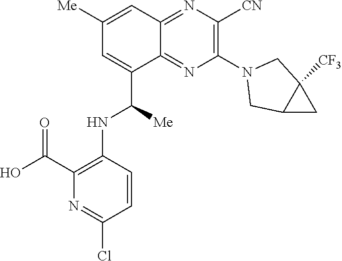 [SMILES: Cc1cc([C@@H](C)Nc2ccc(Cl)nc2C(=O)O)c2nc(N3CC4C[C@]4(C(F)(F)F)C3)c(C#N)nc2c1] |
| (R)-6-chloro-3-((1-(2-cyclopropyl-3-(4,4- difluoropiperidin-1-yl)-7-methylquinoxalin-5- yl)ethyl)amino)picolinic acid | 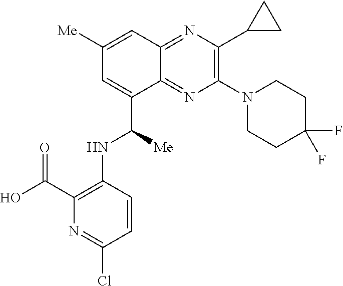 [SMILES: Cc1cc([C@@H](C)Nc2ccc(Cl)nc2C(=O)O)c2nc(N3CCC(F)(F)CC3)c(C3CC3)nc2c1] |
| (R)-6-chloro-3-((1-(3-(4,4-difluoropiperidin-1-yl)-7- methyl-2-(trifluoromethyl)quinoxalin-5- yl)ethyl)amino)picolinic acid | 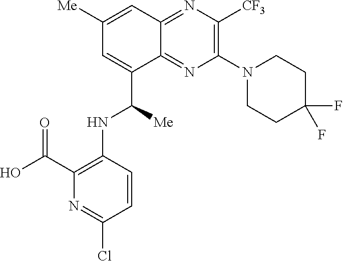 [SMILES: Cc1cc([C@@H](C)Nc2ccc(Cl)nc2C(=O)O)c2nc(N3CCC(F)(F)CC3)c(C(F)(F)F)nc2c1] |
| (R)-2-((1-(3-(4,4-difluoropiperidin-1-yl)-7-methyl-2- (trifluoromethyl)quinoxalin-5-yl)ethyl)amino)benzoic acid | 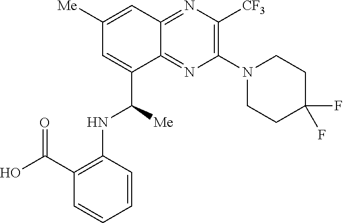 [SMILES: Cc1cc([C@@H](C)Nc2ccccc2C(=O)O)c2nc(N3CCC(F)(F)CC3)c(C(F)(F)F)nc2c1] |
| (R)-2-((1-(2-cyano-3-(4,4-difluoropiperidin-1-yl)-7- ethylquinoxalin-5-yl)ethyl)amino)benzoic acid | 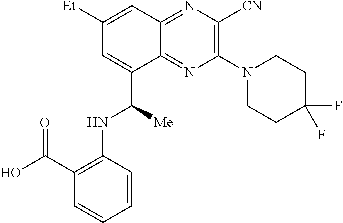 [SMILES: CCc1cc([C@@H](C)Nc2ccccc2C(=O)O)c2nc(N3CCC(F)(F)CC3)c(C#N)nc2c1] |
| (R)-6-chloro-3-((1-(2-chloro-3-(4,4-difluoropiperidin- 1-yl)-7-methylquinoxalin-5-yl)ethyl)amino)picolinic acid | 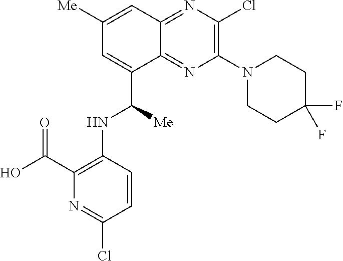 [SMILES: Cc1cc([C@@H](C)Nc2ccc(Cl)nc2C(=O)O)c2nc(N3CCC(F)(F)CC3)c(Cl)nc2c1] |
| (R)-2-((1-(2-chloro-3-(4,4-difluoropiperidin-1-yl)-7- methylquinoxalin-5-yl)ethyl)amino)benzoic acid | 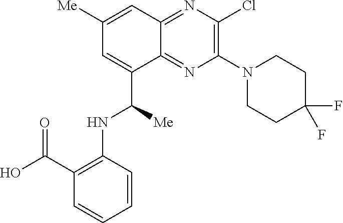 [SMILES: Cc1cc([C@@H](C)Nc2ccccc2C(=O)O)c2nc(N3CCC(F)(F)CC3)c(Cl)nc2c1] |
| (R)-3-((1-(3-(4,4-difluoropiperidin-1-yl)-2,7- dimethylquinoxalin-5-yl)ethyl)amino)-6- methylpicolinic acid | 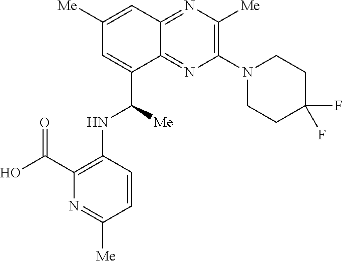 [SMILES: Cc1cc([C@@H](C)Nc2ccc(C)nc2C(=O)O)c2nc(N3CCC(F)(F)CC3)c(C)nc2c1] |
| (R)-2-((1-(2-cyano-3-(3,3-dimethylazetidin-1-yl)-7- methylquinoxalin-5-yl)ethyl)amino)benzoic acid | 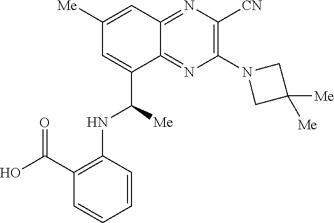 [SMILES: Cc1cc([C@@H](C)Nc2ccccc2C(=O)O)c2nc(N3CC(C)(C)C3)c(C#N)nc2c1] |
| (R)-6-chloro-3-((1-(2-cyano-3-(3,3-dimethylazetidin- 1-yl)-7-methylquinoxalin-5-yl)ethyl)amino)picolinic acid | 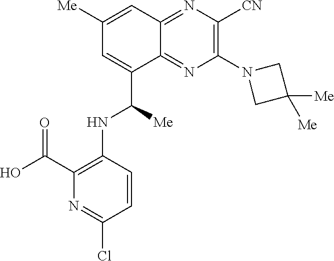 [SMILES: Cc1cc([C@@H](C)Nc2ccc(Cl)nc2C(=O)O)c2nc(N3CC(C)(C)C3)c(C#N)nc2c1] |
| (R)-2-((1-(2-cyano-7-methyl-3-morpholinoquinoxalin- 5-yl)ethyl)amino)benzoic acid | 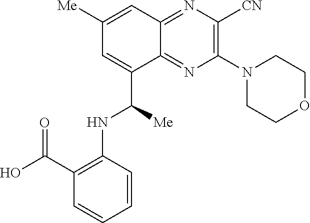 [SMILES: Cc1cc([C@@H](C)Nc2ccccc2C(=O)O)c2nc(N3CCOCC3)c(C#N)nc2c1] |
| (R)-6-chloro-3-((1-(2-cyano-7-methyl-3- morpholinoquinoxalin-5-yl)ethyl)amino)picolinic acid | 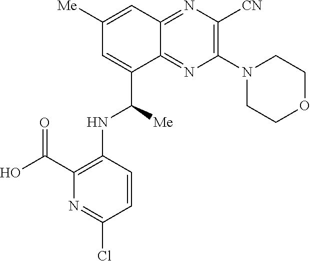 [SMILES: Cc1cc([C@@H](C)Nc2ccc(Cl)nc2C(=O)O)c2nc(N3CCOCC3)c(C#N)nc2c1] |
| (R)-2-((1-(2-cyano-7-methyl-3-(2-oxa-6- azaspiro[3.3]heptan-6-yl)quinoxalin-5- yl)ethyl)amino)benzoic acid | 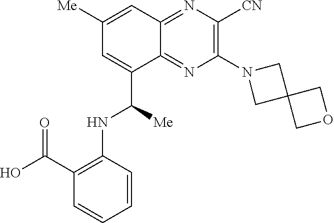 [SMILES: Cc1cc([C@@H](C)Nc2ccccc2C(=O)O)c2nc(N3CC4(COC4)C3)c(C#N)nc2c1] |
| (R)-6-chloro-3-((1-(2-cyano-7-methyl-3-(2-oxa-6- azaspiro[3.3]heptan-6-yl)quinoxalin-5- yl)ethyl)amino)picolinic acid | 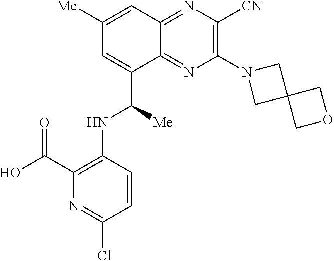 [SMILES: Cc1cc([C@@H](C)Nc2ccc(Cl)nc2C(=O)O)c2nc(N3CC4(COC4)C3)c(C#N)nc2c1] |
| (R)-2-((1-(2-cyano-3-(4,4-dimethylpiperidin-1-yl)-7- methylquinoxalin-5-yl)ethyl)amino)benzoic acid | 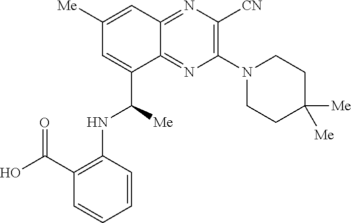 [SMILES: Cc1cc([C@@H](C)Nc2ccccc2C(=O)O)c2nc(N3CCC(C)(C)CC3)c(C#N)nc2c1] |
| (R)-6-chloro-3-((1-(2-cyano-3-(4,4-dimethylpiperidin- 1-yl)-7-methylquinoxalin-5-yl)ethyl)amino)picolinic acid | 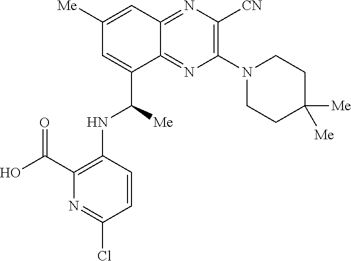 [SMILES: Cc1cc([C@@H](C)Nc2ccc(Cl)nc2C(=O)O)c2nc(N3CCC(C)(C)CC3)c(C#N)nc2c1] |
| (R)-2-((1-(2-cyano-3-(3-methoxy-3-methylazetidin-1- yl)-7-methylquinoxalin-5-yl)ethyl)amino)benzoic acid | 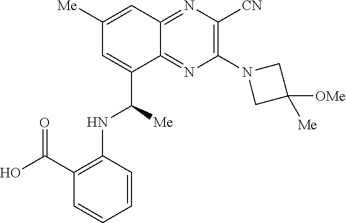 [SMILES: COC1(C)CN(c2nc3c([C@@H](C)Nc4ccccc4C(=O)O)cc(C)cc3nc2C#N)C1] |
| (R)-6-chloro-3-((1-(2-cyano-3-(3-methoxy-3- methylazetidin-1-yl)-7-methylquinoxalin-5- yl)ethyl)amino)picolinic acid | 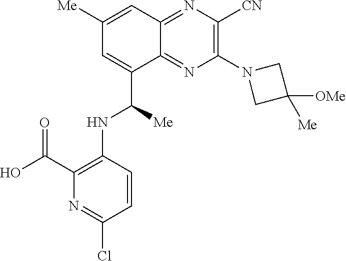 [SMILES: COC1(C)CN(c2nc3c([C@@H](C)Nc4ccc(Cl)nc4C(=O)O)cc(C)cc3nc2C#N)C1] |
| (R)-2-((1-(2-cyano-7-methyl-3-(4-methyl-4- (trifluoromethyl)piperidin-1-yl)quinoxalin-5- yl)ethyl)amino)benzoic acid | 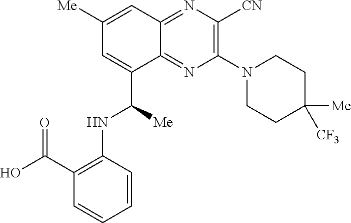 [SMILES: Cc1cc([C@@H](C)Nc2ccccc2C(=O)O)c2nc(N3CCC(C)(C(F)(F)F)CC3)c(C#N)nc2c1] |
| (R)-6-chloro-3-((1-(2-cyano-7-methyl-3-(4-methyl-4- (trifluoromethyl)piperidin-1-yl)quinoxalin-5- yl)ethyl)amino)picolinic acid | 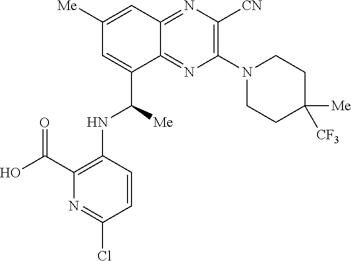 [SMILES: Cc1cc([C@@H](C)Nc2ccc(Cl)nc2C(=O)O)c2nc(N3CCC(C)(C(F)(F)F)CC3)c(C#N)nc2c1] |
| (R)-2-((1-(2-cyano-3-cyclopropyl-7-methylquinoxalin- 5-yl)ethyl)amino)benzoic acid | 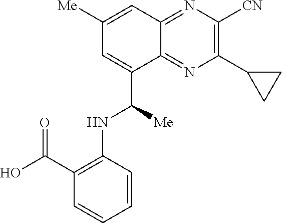 [SMILES: Cc1cc([C@@H](C)Nc2ccccc2C(=O)O)c2nc(C3CC3)c(C#N)nc2c1] |
| (R)-2-((1-(2-cyano-7-methyl-3-(pyrrolidin-1- yl)quinoxalin-5-yl)ethyl)amino)benzoic acid | 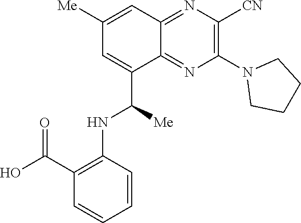 [SMILES: Cc1cc([C@@H](C)Nc2ccccc2C(=O)O)c2nc(N3CCCC3)c(C#N)nc2c1] |
| (R)-6-chloro-3-((1-(2-cyano-7-methyl-3-(pyrrolidin-1- yl)quinoxalin-5-yl)ethyl)amino)picolinic acid | 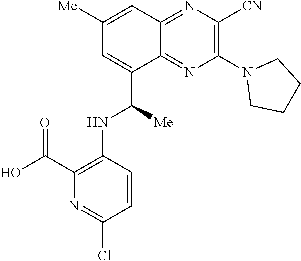 [SMILES: Cc1cc([C@@H](C)Nc2ccc(Cl)nc2C(=O)O)c2nc(N3CCCC3)c(C#N)nc2c1] |
| (R)-6-chloro-3-((1-(2-cyano-3-(3,3-difluoropyrrolidin- 1-yl)-7-methylquinoxalin-5-yl)ethyl)amino)picolinic acid | 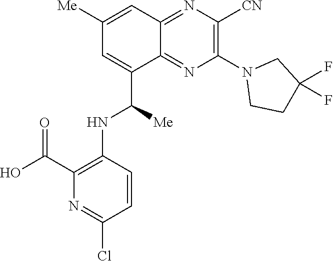 [SMILES: Cc1cc([C@@H](C)Nc2ccc(Cl)nc2C(=O)O)c2nc(N3CCC(F)(F)C3)c(C#N)nc2c1] |
| (R)-2-((1-(2-cyano-3-(3,3-difluoropyrrolidin-1-yl)-7- methylquinoxalin-5-yl)ethyl)amino)benzoic acid | 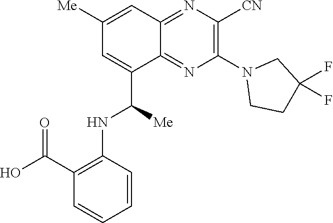 [SMILES: Cc1cc([C@@H](C)Nc2ccccc2C(=O)O)c2nc(N3CCC(F)(F)C3)c(C#N)nc2c1] |
| 2-(((1R)-1-(2-cyano-3-(3-methoxypyrrolidin-1-yl)-7- methylquinoxalin-5-yl)ethyl)amino)benzoic acid | 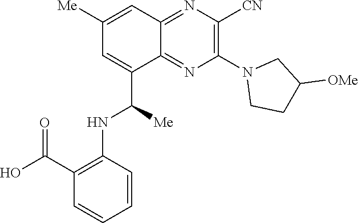 [SMILES: COC1CCN(c2nc3c([C@@H](C)Nc4ccccc4C(=O)O)cc(C)cc3nc2C#N)C1] |
| 6-chloro-3-(((1R)-1-(2-cyano-3-(3-methoxypyrrolidin- 1-yl)-7-methylquinoxalin-5-yl)ethyl)amino)picolinic acid | 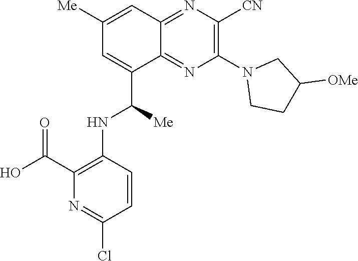 [SMILES: COC1CCN(c2nc3c([C@@H](C)Nc4ccc(Cl)nc4C(=O)O)cc(C)cc3nc2C#N)C1] |
| 6-chloro-3-(((1R)-1-(2-cyano-3-(((2,2-difluoro-1- methylcyclopropyl)methyl)amino)-7- methylquinoxalin-5-yl)ethyl)amino)picolinic acid | 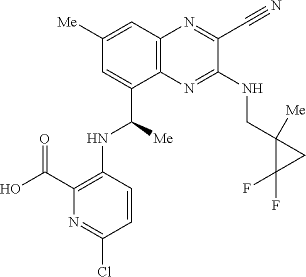 [SMILES: Cc1cc([C@@H](C)Nc2ccc(Cl)nc2C(=O)O)c2nc(NCC3(C)CC3(F)F)c(C#N)nc2c1] |
| 2-(((1R)-1-(2-cyano-3-(((2,2-difluoro-1- methylcyclopropyl)methyl)amino)-7- methylquinoxalin-5-yl)ethyl)amino)benzoic acid | 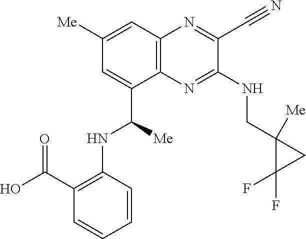 [SMILES: Cc1cc([C@@H](C)Nc2ccccc2C(=O)O)c2nc(NCC3(C)CC3(F)F)c(C#N)nc2c1] |
| (R)-2-((1-(2-cyano-3-((3,3-difluorocyclobutyl)amino)- 7-methylquinoxalin-5-yl)ethyl)amino)benzoic acid | 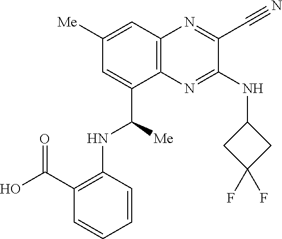 [SMILES: Cc1cc([C@@H](C)Nc2ccccc2C(=O)O)c2nc(NC3CC(F)(F)C3)c(C#N)nc2c1] |
| (R)-6-chloro-3-((1-(2-cyano-3-((3,3- difluorocyclobutyl)amino)-7-methylquinoxalin-5- yl)ethyl)amino)picolinic acid | 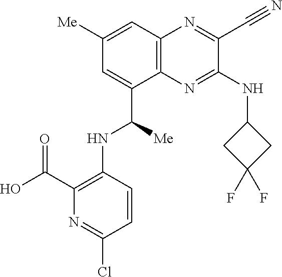 [SMILES: Cc1cc([C@@H](C)Nc2ccc(Cl)nc2C(=O)O)c2nc(NC3CC(F)(F)C3)c(C#N)nc2c1] |
| 6-chloro-3-(((1R)-1-(2-cyano-3-(4,4-difluoro-3- methylpiperidin-1-yl)-7-methylquinoxalin-5- yl)ethyl)amino) picolinic acid | 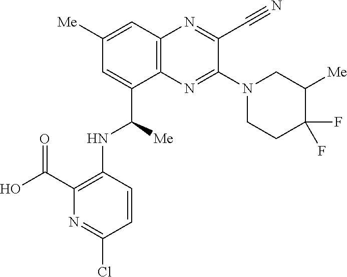 [SMILES: Cc1cc([C@@H](C)Nc2ccc(Cl)nc2C(=O)O)c2nc(N3CCC(F)(F)C(C)C3)c(C#N)nc2c1] |
| 2-(((1R)-1-(2-cyano-3-(4,4-difluoro-3-methylpiperidin- 1-yl)-7-methylquinoxalin-5-yl)ethyl)amino)benzoic acid | 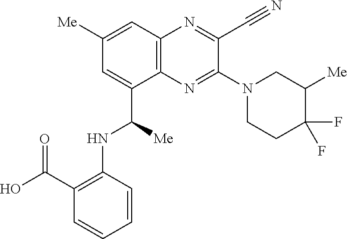 [SMILES: Cc1cc([C@@H](C)Nc2ccccc2C(=O)O)c2nc(N3CCC(F)(F)C(C)C3)c(C#N)nc2c1] |
| 6-chloro-3-(((1R)-1-(2-cyano-3-(3,3-difluoro-4- methylpyrrolidin-1-yl)-7-methylquinoxalin-5- yl)ethyl)amino)picolinic acid | 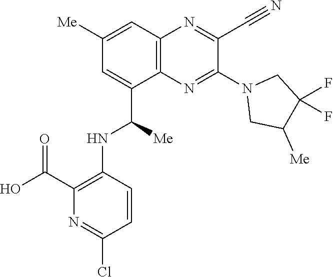 [SMILES: Cc1cc([C@@H](C)Nc2ccc(Cl)nc2C(=O)O)c2nc(N3CC(C)C(F)(F)C3)c(C#N)nc2c1] |
| 2-(((1R)-1-(2-cyano-3-(3,3-difluoro-4- methylpyrrolidin-1-yl)-7-methylquinoxalin-5- yl)ethyl)amino)benzoic acid | 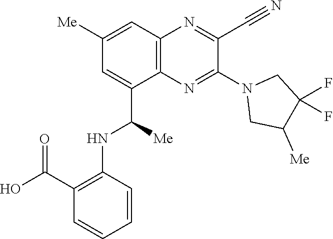 [SMILES: Cc1cc([C@@H](C)Nc2ccccc2C(=O)O)c2nc(N3CC(C)C(F)(F)C3)c(C#N)nc2c1] |
| (R)-6-chloro-3-((1-(2-cyano-7-methyl-3-(1,1,2,2- tetrafluoro-6-azaspiro[2.5]octan-6-yl)quinoxalin-5- yl)ethyl)amino)picolinic acid | 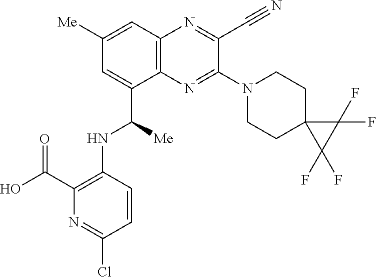 [SMILES: Cc1cc([C@@H](C)Nc2ccc(Cl)nc2C(=O)O)c2nc(N3CCC4(CC3)C(F)(F)C4(F)F)c(C#N)nc2c1] |
| (R)-6-chloro-3-((1-(2-cyano-3-(3,4- dihydroisoquinolin-2(1H)-yl)-7-methylquinoxalin-5- yl)ethyl)amino)picolinic acid | 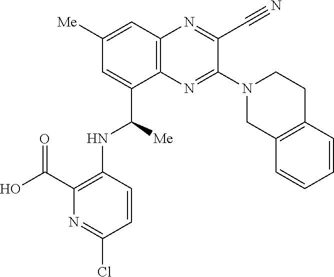 [SMILES: Cc1cc([C@@H](C)Nc2ccc(Cl)nc2C(=O)O)c2nc(N3CCc4ccccc4C3)c(C#N)nc2c1] |
| (R)-2-((1-(2-cyano-3-(3,4-dihydroisoquinolin-2(1H)- yl)-7-methylquinoxalin-5-yl)ethyl)amino)benzoic acid | 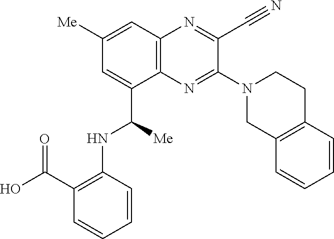 [SMILES: Cc1cc([C@@H](C)Nc2ccccc2C(=O)O)c2nc(N3CCc4ccccc4C3)c(C#N)nc2c1] |
| (R)-6-chloro-3-((1-(2-cyano-3-(6-cyano-3,4- dihydroisoquinolin-2(1H)-yl)-7-methylquinoxalin-5- yl)ethyl)amino)picolinic acid | 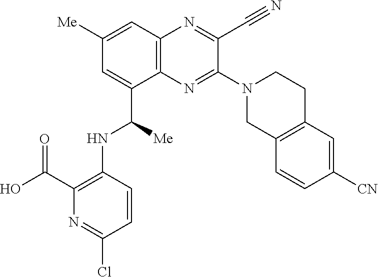 [SMILES: Cc1cc([C@@H](C)Nc2ccc(Cl)nc2C(=O)O)c2nc(N3CCc4cc(C#N)ccc4C3)c(C#N)nc2c1] |
| (R)-2-((1-(2-cyano-3-(6-cyano-3,4- dihydroisoquinolin-2(1H)-yl)-7-methylquinoxalin-5- yl)ethyl)amino)benzoic acid | 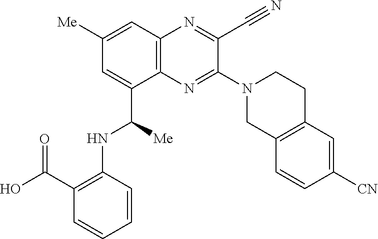 [SMILES: Cc1cc([C@@H](C)Nc2ccccc2C(=O)O)c2nc(N3CCc4cc(C#N)ccc4C3)c(C#N)nc2c1] |
| (R)-6-chloro-3-((1-(2-cyano-3-(6-fluoro-3,4- dihydroisoquinolin-2(1H)-yl)-7-methylquinoxalin-5- yl)ethyl)amino)picolinic acid | 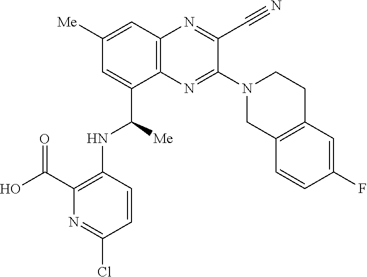 [SMILES: Cc1cc([C@@H](C)Nc2ccc(Cl)nc2C(=O)O)c2nc(N3CCc4cc(F)ccc4C3)c(C#N)nc2c1] |
| (R)-2-((1-(2-cyano-3-(6-fluoro-3,4-dihydroisoquinolin- 2(1H)-yl)-7-methylquinoxalin-5- yl)ethyl)amino)benzoic acid | 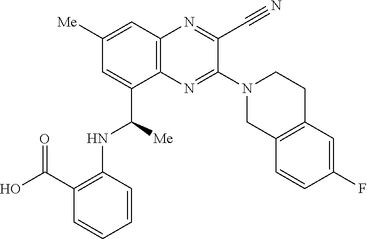 [SMILES: Cc1cc([C@@H](C)Nc2ccccc2C(=O)O)c2nc(N3CCc4cc(F)ccc4C3)c(C#N)nc2c1] |
| (R)-2-((1-(2-cyano-3-(3,4-dihydro-2,7-naphthyridin- 2(1H)-yl)-7-methylquinoxalin-5- yl)ethyl)amino)benzoic acid | 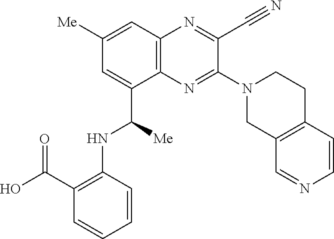 [SMILES: Cc1cc([C@@H](C)Nc2ccccc2C(=O)O)c2nc(N3CCc4ccncc4C3)c(C#N)nc2c1] |
| (R)-6-chloro-3-((1-(2-cyano-3-(4-(2- cyclopropylacetyl)piperazin-1-yl)-7-methylquinoxalin- 5-yl)ethyl)amino)picolinic acid | 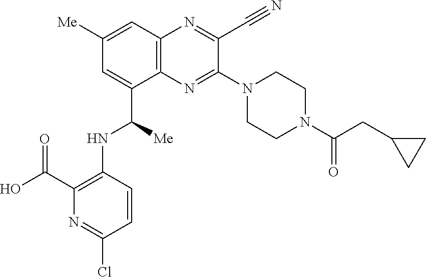 [SMILES: Cc1cc([C@@H](C)Nc2ccc(Cl)nc2C(=O)O)c2nc(N3CCN(C(=O)CC4CC4)CC3)c(C#N)nc2c1] |
| (R)-2-((1-(2-cyano-3-(4-(2- cyclopropylacetyl)piperazin-1-yl)-7-methylquinoxalin- 5-yl)ethyl)amino)benzoic acid | 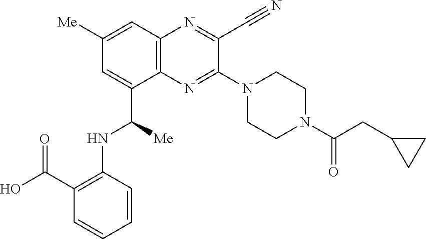 [SMILES: Cc1cc([C@@H](C)Nc2ccccc2C(=O)O)c2nc(N3CCN(C(=O)CC4CC4)CC3)c(C#N)nc2c1] |
| 6-chloro-3-(((1R)-1-(2-cyano-3-(4-(2-(2,2- difluorocyclopropyl)acetyl)piperazin-1-yl)-7- methylquinoxalin-5-yl)ethyl) amino)picolinic acid | 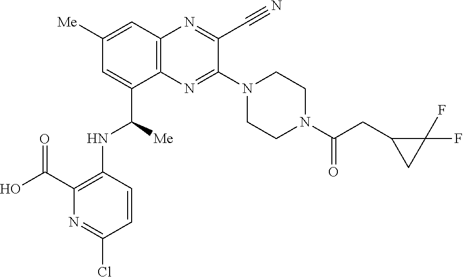 [SMILES: Cc1cc([C@@H](C)Nc2ccc(Cl)nc2C(=O)O)c2nc(N3CCN(C(=O)CC4CC4(F)F)CC3)c(C#N)nc2c1] |
| 2-(((1R)-1-(2-cyano-3-(4-(2-(2,2- difluorocyclopropyl)acetyl)piperazin-1-yl)-7- methylquinoxalin-5-yl)ethyl)amino)benzoic acid | 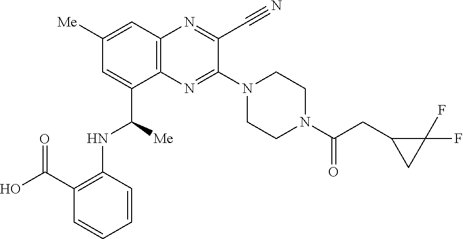 [SMILES: Cc1cc([C@@H](C)Nc2ccccc2C(=O)O)c2nc(N3CCN(C(=O)CC4CC4(F)F)CC3)c(C#N)nc2c1] |
| 6-chloro-3-(((1R)-1-(2-cyano-3-(1-hydroxy-6- azaspiro[3.5]nonan-6-yl)-7-methylquinoxalin-5- yl)ethyl)amino) picolinic acid | 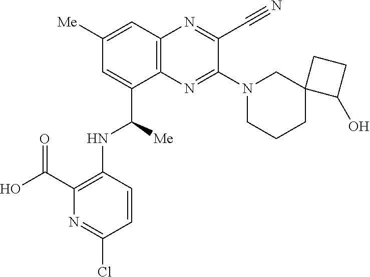 [SMILES: Cc1cc([C@@H](C)Nc2ccc(Cl)nc2C(=O)O)c2nc(N3CCCC4(CCC4O)C3)c(C#N)nc2c1] |
| 2-(((1R)-1-(2-cyano-3-(1-hydroxy-6- azaspiro[3.5]nonan-6-yl)-7-methylquinoxalin-5- yl)ethyl)amino)benzoic acid | 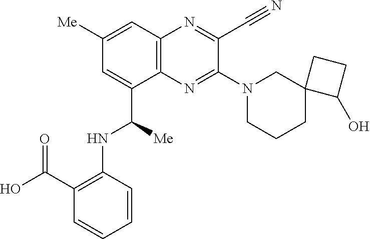 [SMILES: Cc1cc([C@@H](C)Nc2ccccc2C(=O)O)c2nc(N3CCCC4(CCC4O)C3)c(C#N)nc2c1] |
| 6-chloro-3-(((1R)-1-(2-cyano-7-methyl-3-(3-(1- methyl-1H-pyrazol-5-yl)-8-azabicyclo[3.2.1]octan-8- yl)quinoxalin-5-yl)ethyl)amino)picolinic acid | 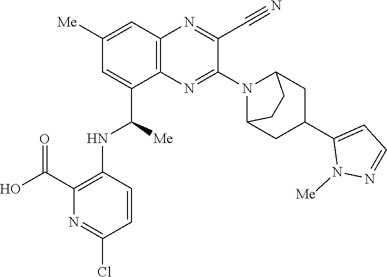 [SMILES: Cc1cc([C@@H](C)Nc2ccc(Cl)nc2C(=O)O)c2nc(N3C4CCC3CC(c3ccnn3C)C4)c(C#N)nc2c1] |
| 2-(((1R)-1-(2-cyano-7-methyl-3-(3-(1-methyl-1H- pyrazol-5-yl)-8-azabicyclo[3.2.1]octan-8- yl)quinoxalin-5-yl)ethyl)amino)benzoic acid | 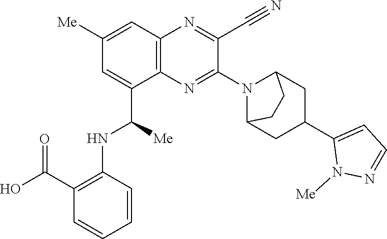 [SMILES: Cc1cc([C@@H](C)Nc2ccccc2C(=O)O)c2nc(N3C4CCC3CC(c3ccnn3C)C4)c(C#N)nc2c1] |
| 2-(((1R)-1-(2-cyano-7-methyl-3-(3-(1-methyl-1H- pyrazol-3-yl)-8-azabicyclo[3.2.1]octan-8- yl)quinoxalin-5-yl)ethyl)amino)benzoic acid | 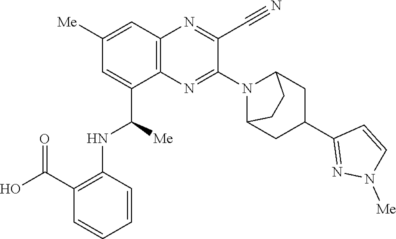 [SMILES: Cc1cc([C@@H](C)Nc2ccccc2C(=O)O)c2nc(N3C4CCC3CC(c3ccn(C)n3)C4)c(C#N)nc2c1] |
| 2-(((1R)-1-(2-cyano-7-methyl-3-(3-(1-methyl-1H- pyrazol-4-yl)-8-azabicyclo[3.2.1]octan-8- yl)quinoxalin-5-yl)ethyl)amino)benzoic acid | 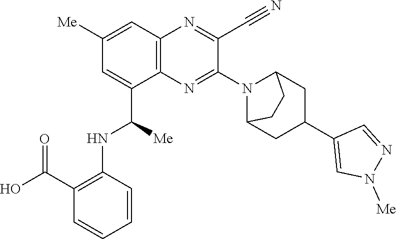 [SMILES: Cc1cc([C@@H](C)Nc2ccccc2C(=O)O)c2nc(N3C4CCC3CC(c3cnn(C)c3)C4)c(C#N)nc2c1] |
| 6-chloro-3-(((1R)-1-(2-cyano-7-methyl-3-(3-(1- methyl-1H-pyrazol-3-yl)-8-azabicyclo[3.2.1]octan-8- yl)quinoxalin-5-yl)ethyl)amino)picolinic acid | 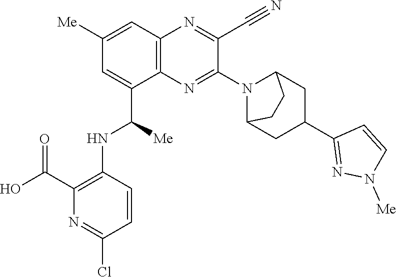 [SMILES: Cc1cc([C@@H](C)Nc2ccc(Cl)nc2C(=O)O)c2nc(N3C4CCC3CC(c3ccn(C)n3)C4)c(C#N)nc2c1] |
| 6-chloro-3-(((1R)-1-(2-cyano-7-methyl-3-(3-(1- methyl-1H-pyrazol-4-yl)-8-azabicyclo[3.2.1]octan-8- yl)quinoxalin-5-yl)ethyl)amino)picolinic acid | 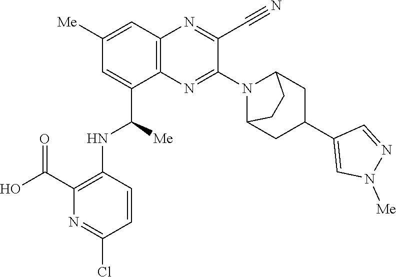 [SMILES: Cc1cc([C@@H](C)Nc2ccc(Cl)nc2C(=O)O)c2nc(N3C4CCC3CC(c3cnn(C)c3)C4)c(C#N)nc2c1] |
| (R)-6-chloro-3-((1-(2-cyano-7-methyl-3-(2-oxo-[1,4'- bipiperidin]-1'-yl)quinoxalin-5-yl)ethyl)amino)picolinic acid | 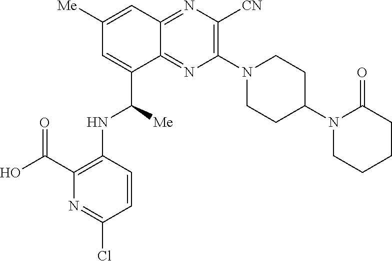 [SMILES: Cc1cc([C@@H](C)Nc2ccc(Cl)nc2C(=O)O)c2nc(N3CCC(N4CCCCC4=O)CC3)c(C#N)nc2c1] |
| 6-chloro-3-(((1R)-1-(2-cyano-7-methyl-3-(3-(2- oxopiperidin-1-yl)-8-azabicyclo[3.2.1]octan-8- yl)quinoxalin-5-yl)ethyl)amino)picolinic acid | 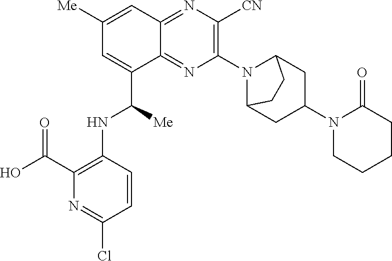 [SMILES: Cc1cc([C@@H](C)Nc2ccc(Cl)nc2C(=O)O)c2nc(N3C4CCC3CC(N3CCCCC3=O)C4)c(C#N)nc2c1] |
| (R)-2-((1-(2-cyano-7-methyl-3-(2-oxo-[1,4'- bipiperidin]-1′-yl)quinoxalin-5-yl)ethyl)amino)benzoic acid | 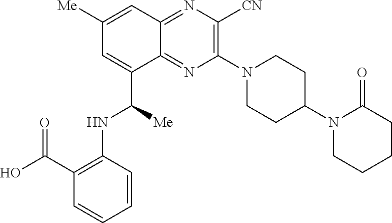 [SMILES: Cc1cc([C@@H](C)Nc2ccccc2C(=O)O)c2nc(N3CCC(N4CCCCC4=O)CC3)c(C#N)nc2c1] |
| (R)-6-chloro-3-((1-(2-cyano-3-(4- (ethoxymethyl)piperidin-1-yl)-7-methylquinoxalin-5- yl)ethyl)amino)picolinic acid | 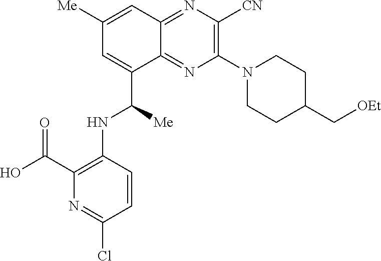 [SMILES: CCOCC1CCN(c2nc3c([C@@H](C)Nc4ccc(Cl)nc4C(=O)O)cc(C)cc3nc2C#N)CC1] |
| (R)-2-((1-(2-cyano-3-(4-(ethoxymethyl)piperidin-1-yl)- 7-methylquinoxalin-5-yl)ethyl)amino)benzoic acid | 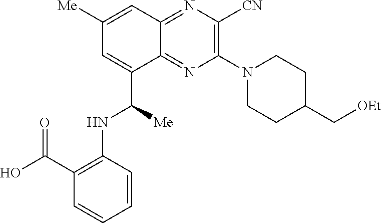 [SMILES: CCOCC1CCN(c2nc3c([C@@H](C)Nc4ccccc4C(=O)O)cc(C)cc3nc2C#N)CC1] |
| or a pharmaceutically acceptable salt thereof. | |

### Pharmaceutical Compositions

The compounds of Formula I may be formulated into pharmaceutical compositions.

In another aspect, the invention provides pharmaceutical compositions comprising a PI3Kα inhibitor according to the invention and a pharmaceutically acceptable carrier, excipient, or diluent. Compounds of the invention may be formulated by any method well known in the art and may be prepared for administration by any route, including, without limitation, parenteral, oral, sublingual, transdermal, topical, intranasal, intratracheal, or intrarectal. In certain embodiments, compounds of the invention are administered intravenously in a hospital setting. In certain other embodiments, administration may preferably be by the oral route.

The characteristics of the carrier will depend on the route of administration. As used herein, the term “pharmaceutically acceptable” means a non-toxic material that is compatible with a biological system such as a cell, cell culture, tissue, or organism, and that does not interfere with the effectiveness of the biological activity of the active ingredient(s). Thus, compositions according to the invention may contain, in addition to the inhibitor, diluents, fillers, salts, buffers, stabilizers, solubilizers, and other materials well known in the art. The preparation of pharmaceutically acceptable formulations is described in, e.g., Remington's Pharmaceutical Sciences, 18th Edition, ed. A. Gennaro, Mack Publishing Co., Easton, Pa., 1990.

As used herein, the term “pharmaceutically acceptable salts” refers to salts that retain the desired biological activity of the above-identified compounds and exhibit minimal or no undesired toxicological effects. Examples of such salts include, but are not limited to acid addition salts formed with inorganic acids (for example, hydrochloric acid, hydrobromic acid, sulfuric acid, phosphoric acid, nitric acid, and the like), and salts formed with organic acids such as acetic acid, oxalic acid, tartaric acid, succinic acid, malic acid, ascorbic acid, benzoic acid, tannic acid, pamoic acid, alginic acid, polyglutamic acid, naphthalenesulfonic acid, naphthalenedisulfonic acid, and polygalacturonic acid. The compounds can also be administered as pharmaceutically acceptable quaternary salts known by those skilled in the art, which specifically include the quaternary ammonium salt of the formula —NR+Z—, wherein R is hydrogen, alkyl, or benzyl, and Z is a counterion, including chloride, bromide, iodide, —O-alkyl, toluenesulfonate, methylsulfonate, sulfonate, phosphate, or carboxylate (such as benzoate, succinate, acetate, glycolate, maleate, malate, citrate, tartrate, ascorbate, benzoate, cinnamoate, mandeloate, benzyloate, and diphenylacetate).

The active compound is included in the pharmaceutically acceptable carrier or diluent in an amount sufficient to deliver to a patient a therapeutically effective amount without causing serious toxic effects in the patient treated. A dose of the active compound for all of the above-mentioned conditions is in the range from about 0.01 to 300 mg/kg, preferably 0.1 to 100 mg/kg per day, more generally 0.5 to about 25 mg per kilogram body weight of the recipient per day. A typical topical dosage will range from 0.01-3% wt/wt in a suitable carrier. The effective dosage range of the pharmaceutically acceptable derivatives can be calculated based on the weight of the parent compound to be delivered. If the derivative exhibits activity in itself, the effective dosage can be estimated as above using the weight of the derivative, or by other means known to those skilled in the art.

The pharmaceutical compositions comprising compounds of the present invention may be used in the methods described herein.

### Methods of Use

In another aspect, the present disclosure generally relates to methods for treating cancer. These methods comprise administering to a subject in need thereof, a therapeutically effective amount of a PI3K inhibitor (e.g., PI3Kα inhibitor or PI3Kα H1047R mutant inhibitor).

In some embodiments, the PI3K inhibitor (e.g., PI3Kα inhibitor or PI3Kα H1047R mutant inhibitor) is a compound of Formula (I), (IIa), (IIb), (IIc) or a pharmaceutically acceptable salt, prodrug, solvate, hydrate, isomer, or tautomer thereof. In particular embodiments, the PI3K inhibitor comprises a compound selected from Table 1.

In another aspect, the present disclosure provides a compound obtainable by, or obtained by, a method for preparing a compound as described herein (e.g., a method comprising one or more steps described in the Schemes).

In another aspect, the present disclosure provides a pharmaceutical composition comprising a compound of Formula (I), (IIa), (IIb), (IIc), or a pharmaceutically acceptable salt, prodrug, solvate, hydrate, isomer, or tautomer thereof, and a pharmaceutically acceptable diluent or carrier.

In another aspect, the present disclosure provides an intermediate as described herein, being suitable for use in a method for preparing a compound as described herein (e.g., the intermediate is selected from the intermediates described in the Examples).

In another aspect, the present disclosure provides a method of modulating PI3K (e.g., PI3Kα) activity (e.g., in vitro or in vivo), comprising contacting a cell with a therapeutically effective amount of a compound of Formula (I), (IIa), (IIb), (IIc), or a pharmaceutically acceptable salt, prodrug, solvate, hydrate, isomer, or tautomer thereof.

In particular embodiments, the PI3K-associated with the disease or disorder has a H1047R mutation. For example, in certain embodiments as otherwise described herein, the compounds have a high selectivity for inhibiting H1047R-mutated PI3Kα compared to wild-type PI3Kα. This unexpected finding suggests that certain compounds may allow targeted inhibition of PI3Kα with a novel binding mechanism compared to conventional wild-type PI3Kα inhibitors. Without wishing to be bound by theory, the H1047R-mutated PI3Kα has a modification distant from the wild-type PI3Kα active site. Accordingly, compounds which are selective for H1047R-mutated PI3Kα over wild-type PI3Kα are not believed to strongly bind at the PI3Kα active site, but rather advantageously are believed to target other binding pockets. As the active site of PI3K-type proteins is thought to be better conserved among different variants, compounds that effectively bind at a position different than the active site may provide high selectivity for PI3Kα inhibition over other PI3K proteins, such as PI3Kβ.

In another aspect, methods of treating cancer comprising administering to a patient having cancer a therapeutically effective amount of a compound of Formula I, pharmaceutically acceptable salts thereof or pharmaceutical compositions comprising the compound or pharmaceutically acceptable salts thereof are provided.

The compositions and methods provided herein may be used for the treatment of a wide variety of cancer including tumors such as prostate, breast, brain, skin, cervical carcinomas, testicular carcinomas, etc. More particularly, cancers that may be treated by the compositions and methods of the invention include, but are not limited to tumor types such as astrocytic, breast, cervical, colorectal, endometrial, esophageal, gastric, head and neck, hepatocellular, laryngeal, lung, oral, ovarian, prostate and thyroid carcinomas and sarcomas. More specifically, these compounds can be used to treat: Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Biliary tract: gall bladder carcinoma, ampullary carcinoma, cholangiocarcinoma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematologic: blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma); Skin: malignant melanoma, basal cell carcinoma, squamous cell carcinoma, Kaposi's sarcoma, moles dysplastic nevi, lipoma, angioma, dermatofibroma, keloids, psoriasis; and Adrenal glands: neuroblastoma. In certain embodiments, the cancer is diffuse large B-cell lymphoma (DLBCL).

In one embodiment, the cancer is selected from hepatocellular carcinoma, breast cancer, skin cancer, bladder cancer, liver cancer, pancreatic cancer, and head and neck cancer,

In another embodiment, the cancer is selected from breast cancer, uterine carcinosarcoma, uterine endometrial carcinoma, colorectal adenocarcinoma, stomach adenocarcinoma, head and neck squamous cell carcinoma, cholangiocarcinoma, esophageal adenocarcinoma, bladder carcinoma, lung squamous cell carcinoma, brain glioma, adrenocortical carcinoma, liver hepatocellular carcinoma, sarcoma, prostate adenocarcinoma, kidney renal cell carcinoma, lung adenocarcinoma, ovarian cystadenocarcinoma, glioblastoma multiforme, melanoma.

Thus, in certain embodiments, this disclosure provides methods of treating cancer comprising administering a compound of Formula (I) a pharmaceutical composition thereof and KRAS inhibitor to a patient in need thereof, wherein the cancer is breast cancer, uterine carcinosarcoma, uterine endometrial carcinoma, colorectal adenocarcinoma, stomach adenocarcinoma, head and neck squamous cell carcinoma, cholangiocarcinoma, esophageal adenocarcinoma, bladder carcinoma, lung squamous cell carcinoma, brain glioma, adrenocortical carcinoma, liver hepatocellular carcinoma, sarcoma, prostate adenocarcinoma, kidney renal cell carcinoma, lung adenocarcinoma, ovarian cystadenocarcinoma, glioblastoma multiforme, melanoma.

In other embodiments, this disclosure provides a methods of treating cancer comprising administering a compound of Formula (I) a pharmaceutical composition thereof and a mutant selective KRAS inhibitor to a patient in need thereof, wherein the cancer is breast cancer, uterine carcinosarcoma, uterine endometrial carcinoma, colorectal adenocarcinoma, stomach adenocarcinoma, head and neck squamous cell carcinoma, cholangiocarcinoma, esophageal adenocarcinoma, bladder carcinoma, lung squamous cell carcinoma, brain glioma, adrenocortical carcinoma, liver hepatocellular carcinoma, sarcoma, prostate adenocarcinoma, kidney renal cell carcinoma, lung adenocarcinoma, ovarian cystadenocarcinoma, glioblastoma multiforme, melanoma.

In another embodiment, this disclosure provides a compound of Formula (I) a pharmaceutical composition thereof for use in the treatment of cancer in combination with a KRAS inhibitor, wherein the cancer is breast cancer, uterine carcinosarcoma, uterine endometrial carcinoma, colorectal adenocarcinoma, stomach adenocarcinoma, head and neck squamous cell carcinoma, cholangiocarcinoma, esophageal adenocarcinoma, bladder carcinoma, lung squamous cell carcinoma, brain glioma, adrenocortical carcinoma, liver hepatocellular carcinoma, sarcoma, prostate adenocarcinoma, kidney renal cell carcinoma, lung adenocarcinoma, ovarian cystadenocarcinoma, glioblastoma multiforme, melanoma.

The concentration and route of administration to the patient will vary depending on the cancer to be treated. The compounds, pharmaceutically acceptable salts thereof and pharmaceutical compositions comprising such compounds and salts also may be co-administered with other anti-neoplastic compounds, e.g., chemotherapy, or used in combination with other treatments, such as radiation or surgical intervention, either as an adjuvant prior to surgery or post-operatively.

The present disclosure provides methods of treating, preventing, or ameliorating a disease or disorder in which PI3K plays a role by administering to a patient in need thereof a therapeutically effective amount of a PI3K inhibitor. The methods of the present disclosure can be used in the treatment of a variety of PI3K-dependent diseases and disorders.

In some embodiments, the disease of disorder is a cancer (e.g., breast cancer, brain cancers, prostate cancer, endometrial cancer, gastric cancer, leukemia, lymphoma, sarcoma, colorectal cancer, lung cancer, ovarian cancer, skin cancer, and head and neck cancer). In some embodiments, the disease or disorder associated with PI3K includes, but is not limited to, CLOVES syndrome (congenital lipomatous overgrowth, vascular malformations, epidermal naevi, scoliosis/skeletal and spinal syndrome), PIK3CA-related overgrowth syndrome (PROS), endometrial cancer, breast cancer, esophageal squamous-cell cancer, cervical squamous-cell carcinoma, cervical adenocarcinoma, colorectal adenocarcinoma, bladder urothelial carcinoma, glioblastoma, ovarian cancer, non-small-cell lung cancer, esophagogastric cancer, nerve-sheath tumor, head and neck squamous-cell carcinoma, melanoma, esophagogastric adenocarcinoma, soft-tissue sarcoma, prostate cancer, fibrolamellar carcinoma, hepatocellular carcinoma, diffuse glioma, colorectal cancer, pancreatic cancer, cholangiocarcinoma, B-cell lymphoma, mesothelioma, adrenocortical carcinoma, renal non-clear-cell carcinoma, renal clear-cell carcinoma, germ-cell carcinoma, thymic tumor, pheochromocytoma, miscellaneous neuroepithelial tumor, thyroid cancer, leukemia, and encapsulated glioma.

The details of the disclosure are set forth in the accompanying description below. Although methods and materials similar or equivalent to those described herein can be used in the practice or testing of the present disclosure, illustrative methods and materials are now described. Other features, objects, and advantages of the disclosure will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this disclosure belongs. All patents and publications cited in this specification are incorporated herein by reference in their entireties.

### GENERAL REACTION SCHEMES, INTERMEDIATES AND EXAMPLES

The compounds of the present invention may be prepared using commercially available reagents and intermediates in the synthetic methods and reaction schemes described herein, or may be prepared using other reagents and conventional methods well known to those skilled in the art.

Where reference is made to “first eluting enantiomer” or “second eluting enantiomer”, unless otherwise specifically indicated, the specific stereochemistry of such enantiomers has not been determined and any stereochemistry depicted in the corresponding compound structures has been arbitrarily assigned.

For instance, intermediates for preparing compounds and compounds of Formula (I) of the present invention may be prepared according to General Reaction Schemes 1 and 2:

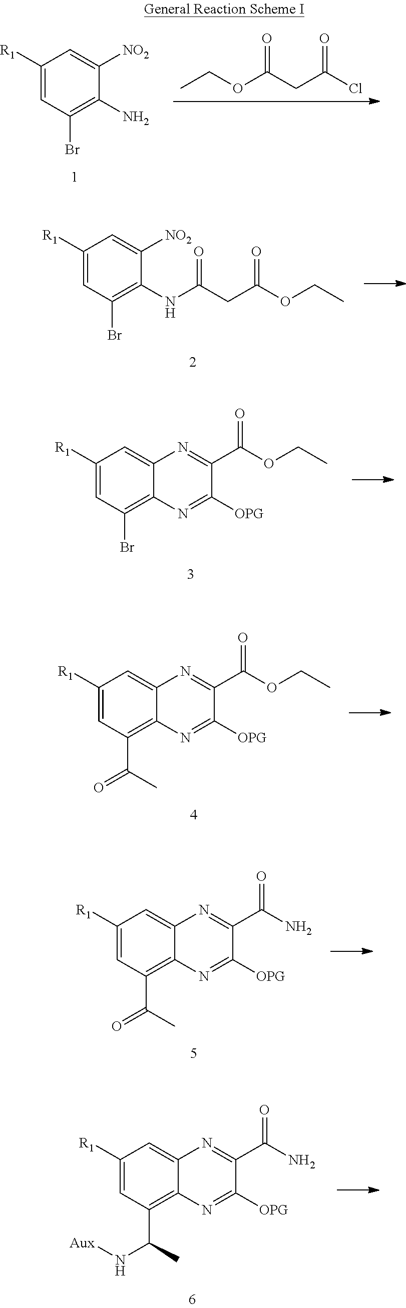

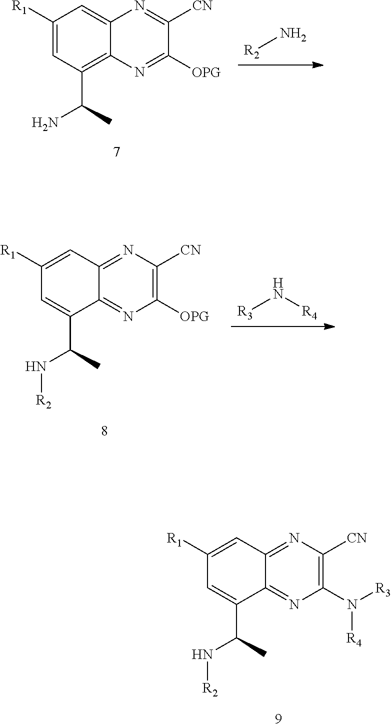

For General Reaction Scheme I, Compound 9 is an example of Formula (I). In this General Reaction Scheme I, 1 can undergo amide bond formation with an activated acid or acid chloride in the presence of a suitable base such as triethylamine or diisopropylethylamine to provide compound 2. In the presence of a suitable base such as sodium ethoxide under heating, condensation to the quinoxaline N-oxide can be accomplished followed by reduction of the N-oxide with a suitable reagent such as phosphorus tribromide and protection to provide compound 3. The aryl bromide can be converted to an acyl group through a palladium catalyzed reaction such as a Heck reaction or Stille with a vinyl ether and subsequent treatment with aqueous acid to provide compound 4. Ammonolysis can be accomplished under heating in a suitable solvent such as ammonia in methanol to prepare compound 5. Condensation of the aryl ketone with an amine bearing a chiral auxiliary followed by reduction to the benzylic amine can be effected by dehydrating reagents such as titanium (IV) ethoxide and hydride transfer reagents such as sodium borohydride, sodium cyanoborohydride, lithium aluminum hydride, diisobutylaluminum hydride, or zirconcocene hydrochloride. Alternatively, compound 6 can be accessed through an asymmetric hydrogenation or transfer hydrogenation or resolution of the racemic amine. The terminal amide can be converted to the nitrile through the action of a dehydrating reagent such as phosphorous oxychloride or Burgess's reagent and deprotection of the benzylic amine can be accomplished under Brønsted or Lewis acidic regents such as hydrochloric acid or trimethylsilyl trifluoromethanesulfonate, for example, to access compound 7. The benzylic amine can be substituted to access compound 8 through the action of palladium or copper catalysis under Buchwald-Hartwig, Ullmann-Goldberg, or Chan-Evans-Lam reaction conditions with a suitable electrophile or boronic acid, solvent and base. Additionally, compound 8 can be accessed through uncatalyzed nucleophilic aromatic substitution reactions with suitable electrophiles at elevated temperatures in the presence of a suitable base, such as diisopropylethylamine. Finally, compounds such as 9 can be accessed after deprotection under acidic conditions such as through the action of trifluoroacetic acid and subsequent activation through the action of benzotriazol-1-yloxytripyrrolidinophosphonium hexafluorophosphate, phosphorous oxychloride or other activating reagent and reaction with a substituted amine in the presence of a base at ambient or elevated temperatures.

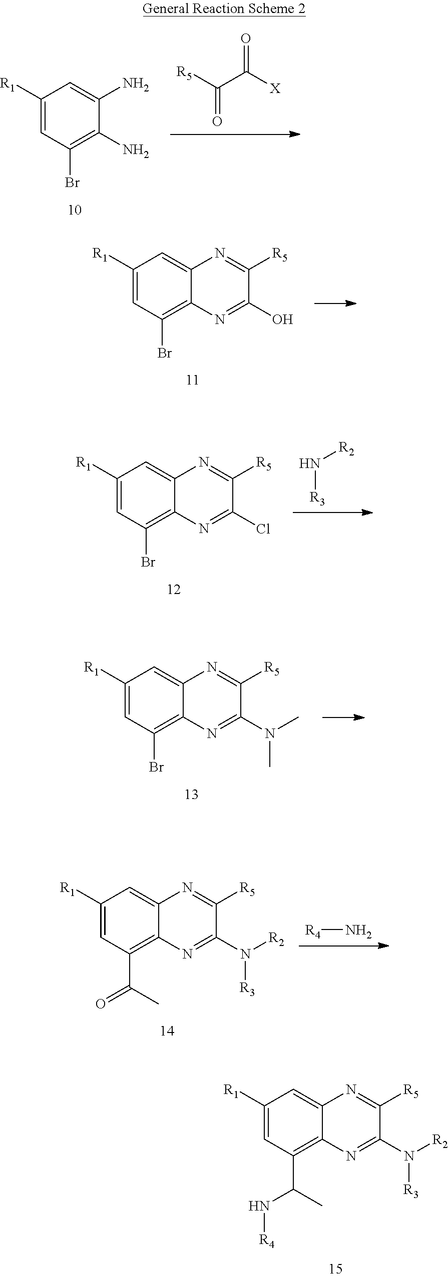

For General Reaction Scheme 2, Compound 15 is an example of Formula (I). In this General Reaction Scheme 2, 10 can be reacted with an activated alpha-keto acid, acid halide, or 2-oxomalonate in a condensation reaction to access compound 11. In the event R₅ is an ester, an aminolysis using a suitable solvent such as ammonia in methanol at elevated temperatures can be used to provide the free amide. Upon treatment of a suitable chlorinating reagent such as phosphorous oxychloride compound 12 can be prepared. Under such conditions, the terminal amide will be dehydrated to provide a nitrile at R₅. Aryl chloride 12 can be reacted with an amine and suitable base, such as diisopropylethylamine, at ambient or elevated temperatures to prepare compound 13. A palladium catalyzed transformation such as a Heck reaction or Stille coupling with a suitable vinyl ether partner and subsequent treatment with an aqueous acid, for example, hydrochloric acid, can provide compound 14. A reductive amination of this ketone and an amine can be effected with a suitable Lewis acid and hydride donor, such as trimethylsilyl trifluoromethanesulfonate and borane, or other common hydride reductant to access compound 15. Alternatively, a sequence analogous to General Reaction Scheme 1 can be used to access a chiral benzylic amine through condensation of a chiral amine reagent and subsequent reduction and carbon-nitrogen bond formation reaction such as a Buchwald-Hartwig reaction, Ullmann-Goldberg Coupling, Chan-Evans-Lam coupling, alkylation, nucleophilic aromatic substitution or reductive amination to provide compound 15.

### Intermediate A

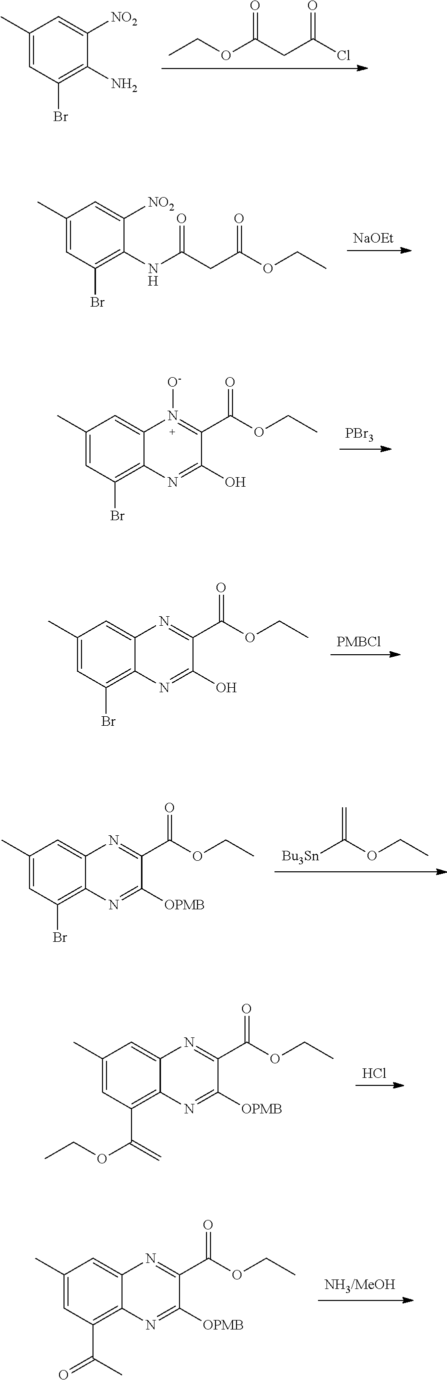

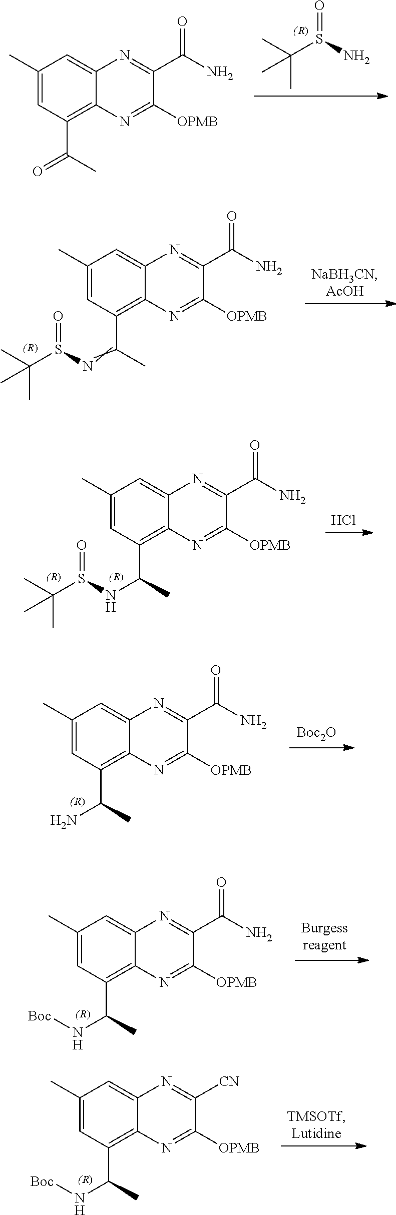

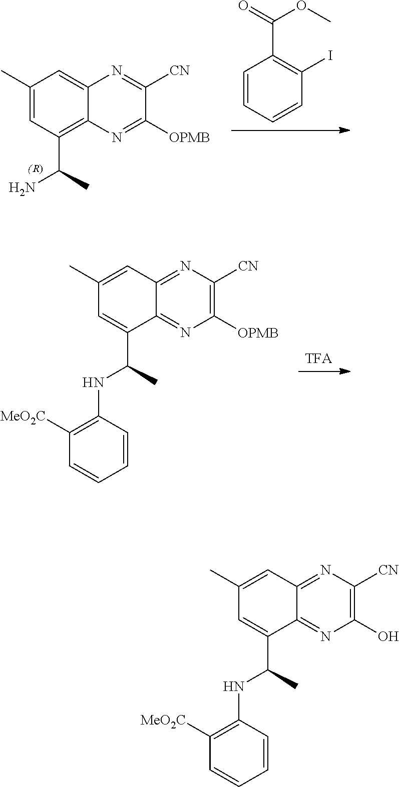

Step A: To a solution of 2-bromo-4-methyl-6-nitroaniline (540 g, 2.34 mol, 1.00 eq.) in toluene (650 mL) was added ethyl 3-chloro-3-oxopropanoate (422 g, 2.80 mol, 353 mL, 1.20 eq.) at 0° C., then the reaction was stirred at 110° C. for 1 hour. After completion of the reaction, the mixture was cooled to 25° C. and poured into petroleum ether (3.00 L). The mixture was filtered and the filtered cake was collected to give ethyl 3-((2-bromo-4-methyl-6-nitrophenyl)amino)-3-oxopropanoate (790 g, 2.29 mol, 97.9% yield) as a yellow solid. LCMS [M+1]⁺=344.9.

¹H NMR (400 MHz, CDCl₃) δ=9.70 (br s, 1H), 7.70 (d, J=2.0 Hz, 2H), 4.30 (q, J=7.2 Hz, 2H), 3.51 (s, 2H), 2.41 (s, 3H), 1.35 (t, J=7.2 Hz, 3H).

Step B: To a solution of sodium ethoxide (734 mL, 2.17 M, 2.20 eq.) in ethanol (500 mL) and tetrahydrofuran (1.50 L) was added a solution of ethyl 3-((2-bromo-4-methyl-6-nitrophenyl)amino)-3-oxopropanoate (250 g, 724 mmol, 1.00 eq.) in tetrahydrofuran (1.00 L) dropwise at −30° C. Then the reaction was stirred at −30° C. for 15 minutes. After completion of the reaction, the mixture was poured into ice water (5.00 L) slowly at 0° C. Then the pH of the mixture was adjusted with hydrogen chloride (4 M in water) to pH˜4. The resulting suspension was filtered and the filtered cake was collected to give a crude product. The crude product was triturated with acetonitrile (750 mL) at room temperature for 30 minutes. Then the mixture was filtered, and the filter cake was collected, dried over under reduced pressure to give 5-bromo-2-(ethoxycarbonyl)-3-hydroxy-7-methylquinoxaline 1-oxide (190 g, 542 mmol, 74.9% yield, 93.4% purity) as a yellow solid. LCMS [M+3]⁺=328.9.

¹H NMR (400 MHz, CDCl₃) δ=9.67 (br s, 1H), 8.09 (s, 1H), 7.71 (d, J=1.2 Hz, 1H), 4.52 (q, J=7.2 Hz, 2H), 2.46 (s, 3H), 1.43 (t, J=6.8 Hz, 3H).

Step C: To a solution of 5-bromo-2-(ethoxycarbonyl)-3-hydroxy-7-methylquinoxaline 1-oxide (200 g, 581 mmol, 1.00 eq.) in DMF (2.50 L) was added phosphorus tribromide (314 g, 1.16 mol, 110 mL, 2.00 eq.) dropwise at 0° C. Then the reaction was stirred at 60° C. for 2 hours. The mixture was then cooled to 25° C. and poured into ice water (4.00 L). The resulting mixture was filtered and the filter cake was collected. The filter cake was triturated with acetonitrile (500 mL) at 15° C. for 30 minutes. Then the mixture was filtered, and the filter cake was collected, dried over under reduced pressure to give ethyl 5-bromo-3-hydroxy-7-methylquinoxaline-2-carboxylate (150 g, 465 mmol, 80.1% yield, 96.5% purity) as a yellow solid. LCMS [M+1]⁺=310.9.

¹H NMR (400 MHz, CDCl₃) δ=9.54 (br s, 1H), 7.71 (s, 1H), 7.66 (s, 1H), 4.51 (q, J=7.2 Hz, 2H), 2.45 (s, 3H), 1.44 (t, J=7.2 Hz, 3H).

Step D: To a solution of ethyl 5-bromo-3-hydroxy-7-methylquinoxaline-2-carboxylate (300 g, 964 mmol, 1.00 eq.) and potassium carbonate (400 g, 2.89 mol, 3.000 eq.) in DMF (2.40 L) was added PMBCI (151 g, 964 mmol, 131 mL, 1.00 eq.), and the reaction was stirred at 50° C. for 8 hours. The mixture was then cooled to 25° C. and then poured into ice water (4.00 L). Then the mixture was filtered, and the filter cake was collected. The filter cake was triturated with acetonitrile (800 mL) at 20° C. for 20 minutes, then filtered and the filter cake was again collected, and dried over under reduced pressure to give ethyl 5-bromo-3-((4-methoxybenzyl)oxy)-7-methylquinoxaline-2-carboxylate (380 g, 819 mmol, 85.0% yield, 93.0% purity) as a yellow solid. LCMS [M+23]⁺=453.1.

¹H NMR (400 MHz, CDCl₃) δ=7.86 (s, 1H), 7.79 (s, 1H), 7.56 (d, J=8.4 Hz, 2H), 6.89 (d, J=8.4 Hz, 2H), 5.61 (s, 2H), 4.49 (q, J=6.8 Hz, 2H), 3.78 (s, 3H), 2.49 (s, 3H), 1.41 (t, J=7.2 Hz, 3H).

Step E: A mixture of ethyl 5-bromo-3-((4-methoxybenzyl)oxy)-7-methylquinoxaline-2-carboxylate (1.00 kg, 2.16 mol, 1.00 eq.), tributyl(1-ethoxyvinyl)tin (1.00 kg, 2.77 mol, 935 mL, 1.28 eq.), Pd₂(dba)₃ (98.7 g, 108 mmol, 0.050 eq.) and XPhos (51.4 g, 108 mmol, 0.050 eq.) in toluene (16.0 L) was degassed and purged with nitrogen 3 times. The reaction was then stirred at 100° C. for 5 hours under a nitrogen atmosphere. After completion of the reaction, the mixture was cooled to 25° C. and then the solution of ethyl 5-(1-ethoxyvinyl)-3-((4-methoxybenzyl)oxy)-7-methylquinoxaline-2-carboxylate (911 g, crude) in toluene (16.0 L) was obtained as a black liquid, which was used directly in the next step. LCMS [M+1]⁺=423.2.

Step F: A solution of ethyl 5-(1-ethoxyvinyl)-3-((4-methoxybenzyl)oxy)-7-methylquinoxaline-2-carboxylate (911 g, crude) in toluene (16.0 L) was cooled to 20° C. and tetrahydrofuran (14.0 L) was added. To this mixture was added hydrogen chloride (1.94 L, 1.00 M, 0.900 eq.) dropwise at 0° C., and the mixture was stirred at 0° C. for 30 minutes. After this time, the pH of the mixture was adjusted with sodium hydrogen carbonate (saturated in water) to pH˜7. To this mixture was added potassium fluoride (4.31 L, 4.00 M in water). After 1 hour, the mixture was diluted with water (6.00 L) and ethyl acetate (6.00 L). The mixture was then filtered, and the filtrate was extracted with ethyl acetate (5.00 Lx 2). The combined organic layers were dried over anhydrous sodium sulfate, filtered, and concentrated under reduced pressure to give a residue. The residue was triturated with (dichloromethane:ethyl acetate=1:2, 500 mL) and then further triturated with (petroleum ether:ethyl acetate=3:1, 500 mL) at 20° C. for 30 minutes. The mixture was then filtered and the filter cake was collected, and dried under reduced pressure to give ethyl 5-acetyl-3-((4-methoxybenzyl)oxy)-7-methylquinoxaline-2-carboxylate (640 g, 1.51 mol, 70.0% yield) as a yellow solid. LCMS [M+23]⁺=417.1.

¹H NMR (400 MHz, CDCl₃) δ=8.04 (d, J=0.8 Hz, 1H), 7.99 (d, J=2.0 Hz, 1H), 7.42 (d, J=8.8 Hz, 2H), 6.92 (d, J=8.4 Hz, 2H), 5.53 (s, 2H), 4.51 (q, J=6.8 Hz, 2H), 3.82 (s, 3H), 2.93 (s, 3H), 2.57 (s, 3H), 1.41 (t, J=7.2 Hz, 3H).

Step G: Ammonia (130 g, 7.62 mol, 14.6 eq.) was bubbled into a solvent of methanol (1.00 L) at 15° C. for 1 hour. Then the above solution was added to a mixture of ethyl 5-acetyl-3-((4-methoxybenzyl)oxy)-7-methylquinoxaline-2-carboxylate (206 g, 522 mmol, 1.00 eq.) in methanol (2.00 L). The reaction was stirred at 25° C. for 16 hours, then concentrated at 30° C. under reduced pressure to give 5-acetyl-3-((4-methoxybenzyl)oxy)-7-methylquinoxaline-2-carboxamide (210 g, 466 mmol, 89.1% yield, 81.0% purity) as a yellow solid. LCMS [M+1]⁺=366.3.

Step H: To a mixture of 5-acetyl-3-((4-methoxybenzyl)oxy)-7-methylquinoxaline-2-carboxamide (89.0 g, 244 mmol, 1.00 eq.) and (R)-2-methylpropane-2-sulfinamide (148 g, 1.22 mol, 5.00 eq.) in 2-methyltetrahydrofuran (3.00 L) was added titanium (IV) ethoxide (278 g, 1.22 mol, 253 mL, 5.00 eq.), and the reaction was stirred at 70° C. for 24 hours. The mixture was then cooled to 25° C., diluted with ethyl acetate (2.00 L) and quenched using sodium hydrogen carbonate (saturated in water, 1.50 L) at 0° C. The mixture was then filtered and the filtrate was washed with water (1.00 L×2), followed by brine (500 mL×1), dried over anhydrous sodium sulfate, filtered, and concentrated under reduced pressure to give a residue. The residue was triturated with ethyl acetate (100 mL) at 20° C. for 1 hour. Then the mixture was filtered and the filter cake was collected, dried over under reduced pressure to give (R)-5-(1-((tert-butylsulfinyl)imino)ethyl)-3-((4-methoxybenzyl)oxy)-7-methylquinoxaline-2-carboxamide (101 g, 174 mmol, 71.4% yield, 80.7% purity) as a yellow solid. LCMS [M+1]⁺=469.2.

¹H NMR (400 MHz, CDCl₃) δ=7.98 (s, 1H), 7.73 (s, 1H), 7.59 (br s, 1H), 7.45-7.42 (m, 2H), 6.91 (br d, J=6.8 Hz, 2H), 6.23 (br s, 1H), 5.55 (s, 2H), 3.81 (s, 3H), 2.94 (d, J=1.6 Hz, 3H), 2.57 (s, 3H), 1.23 (s, 9H).

Step I: To a solution of (R)-5-(1-((tert-butylsulfinyl)imino)ethyl)-3-((4-methoxybenzyl)oxy)-7-methylquinoxaline-2-carboxamide (155 g, 257 mmol, 1.00 eq.) in dichloromethane (1.50 L) and methanol (1.50 L) were added acetic acid (77.1 g, 1.28 mol, 73.5 mL, 5.00 eq.) and sodium cyanoborohydride (64.5 g, 1.03 mol, 4.00 eq.) at 0° C. The reaction was then stirred at 20° C. for 12 hours. After this time, the mixture was washed with water (1.00 L) and the organic phase was treated with sodium hydrogen carbonate (saturated in water, 800 mL) slowly at 0° C. The resultant mixture was washed with water (1.00 L) and brine (500 mL), dried over anhydrous sodium sulfate, filtered, and concentrated under reduced pressure to give 5-((R)-1-(((R)-tert-butylsulfinyl)amino)ethyl)-3-((4-methoxybenzyl)oxy)-7-methylquinoxaline-2-carboxamide (155 g, crude) as a yellow solid. LCMS [M+1]⁺=471.2.

Step J: To a solution of 5-((R)-1-(((R)-tert-butylsulfinyl)amino)ethyl)-3-((4-methoxybenzyl)oxy)-7-methylquinoxaline-2-carboxamide (190 g, 299 mmol, 1.00 eq.) in THF (2.00 L) was added hydrogen chloride (380 mL, 1.00 M in water, 1.27 eq.) dropwise at 0° C., and the mixture was stirred at 15° C. for 7 hours. After the reaction was completed the solution of (R)-5-(1-aminoethyl)-3-((4-methoxybenzyl)oxy)-7-methylquinoxaline-2-carboxamide (160 g, crude) in THF (2.00 L) was obtained as yellow liquid, which was used directly in the next step. LCMS [M+1]⁺=367.1.

Step K: To a solution of ((R)-5-(1-aminoethyl)-3-((4-methoxybenzyl)oxy)-7-methylquinoxaline-2-carboxamide (160 g, 288 mmol, 1.00 eq.) in THF (2.00 L) was added sodium hydroxide (34.6 g, 865 mmol, 3.00 eq.) in water (1.00 L) and Boc₂O (75.5 g, 346 mmol, 79.4 mL, 1.20 eq.), and the reaction was stirred at 50° C. for 1 hour. After this time, the mixture was diluted with ethyl acetate (1.00 L), washed with brine (500 mL×2), dried over anhydrous sodium sulfate, filtered, and concentrated under reduced pressure to give tert-butyl (R)-(1-(2-carbamoyl-3-((4-methoxybenzyl)oxy)-7-methylquinoxalin-5-yl)ethyl)carbamate (160 g, crude) as a yellow solid. LCMS [M+1]⁺=467.3.

Step L: To a solution of tert-butyl (R)-(1-(2-carbamoyl-3-((4-methoxybenzyl)oxy)-7-methylquinoxalin-5-yl)ethyl)carbamate (215 g, 277 mmol, 1.00 eq.) in THF (3.00 L) was added Burgess reagent (198 g, 830 mmol, 3.00 eq.) at 0° C. The reaction was stirred at 0° C. for 1 hour, then diluted with ethyl acetate (1.00 L), washed with water (500 mL) and brine (500 mL×2). The organic phase was dried over anhydrous sodium sulfate, filtered, and concentrated under reduced pressure to give a residue. The residue was triturated with acetonitrile (180 mL) at 20° C. for 30 minutes, then filtered and the filter cake was collected, and dried over reduced pressure to give tert-butyl (R)-(1-(2-cyano-3-((4-methoxybenzyl)oxy)-7-methylquinoxalin-5-yl)ethyl)carbamate (125 g, 234 mmol, 84.7% yield, 84% purity) as a yellow solid. LCMS [M+23]+=471.3.

¹H NMR (400 MHz, CDCl₃) δ=7.73 (d, J=0.8 Hz, 1H), 7.60 (d, J=1.6 Hz, 1H), 7.52-7.47 (m, 2H), 6.97-6.91 (m, 2H), 5.69-5.40 (m, 4H), 3.82 (s, 3H), 2.54 (s, 3H), 1.53 (d, J=6.8 Hz, 3H), 1.44 (br s, 9H).

Step M: To a solution of tert-butyl (R)-(1-(2-cyano-3-((4-methoxybenzyl)oxy)-7-methylquinoxalin-5-yl)ethyl)carbamate (300 g, 562 mmol, 1.00 eq.) in dichloromethane (3.00 L) was added 2,6-dimethylpyridine (482 g, 4.49 mol, 524 mL, 8.00 eq.) and trimethylsilyl trifluoromethanesulfonate (500 g, 2.25 mol, 406 mL, 4.00 eq.), then the mixture was stirred at 40° C. for 1 hour, followed by dilution with water (2.50 L) at 0° C. The resulting mixture was washed with brine (500 mL×2) and then citric acid (1 N in water, 1000 mL×4). The combined organic layers were dried over anhydrous sodium sulfate, filtered, and concentrated under reduced pressure to give a residue. The residue was triturated with acetonitrile (450 mL) at 20° C. for 10 minutes. Then the mixture was filtered, and the filter cake was collected, and dried under reduced pressure to give the crude product. The crude product was purified by reversed phase flash (column: Sfar C18 1800 g D Duo 30 um; mobile phase: phase A: 0.225% formic acid in water, phase B: acetonitrile; gradient: 35%-65% phase B) to give (R)-5-(1-aminoethyl)-3-((4-methoxybenzyl)oxy)-7-methylquinoxaline-2-carbonitrile (195 g, 535 mmol, 95.2% yield, 95.6% purity) as a yellow solid. LCMS [M+1]⁺=349.1.

¹H NMR (400 MHz, CDCl₃) δ=7.71 (s, 2H), 7.47 (d, J=8.4 Hz, 2H), 6.92 (d, J=8.4 Hz, 2H), 5.55 (s, 2H), 5.01 (q, J=6.8 Hz, 1H), 3.81 (s, 3H), 2.69 (br s, 2H), 2.54 (s, 3H), 1.55 (d, J=6.8 Hz, 3H).

Step N: A mixture of (R)-5-(1-aminoethyl)-3-((4-methoxybenzyl)oxy)-7-methylquinoxaline-2-carbonitrile (40.0 g, 115 mmol, 1.00 eq.), methyl 2-iodobenzoate (45.1 g, 172 mmol, 25.3 mL, 1.50 eq.), Pd₂(dba)₃ (5.26 g, 5.74 mmol, 0.050 eq.), XantPhos (3.32 g, 5.74 mmol, 0.050 eq.) and cesium carbonate (112 g, 344 mmol, 3.00 eq.) in 1,4-dioxane (400 mL) was degassed and purged with nitrogen 3 times. The mixture was then stirred at 100° C. for 5 hours under a nitrogen atmosphere. After completion of the reaction, the mixture was filtered, and the filtrate was concentrated under reduced pressure to give a residue. The residue was triturated with a mixture solvent of petroleum ether:ethyl acetate:acetonitrile=3:1:1 (80.0 mL) at 20° C. for 10 minutes, then the mixture was filtered and the filter cake was collected, dried under reduced pressure to give methyl (R)-2-((1-(2-cyano-3-((4-methoxybenzyl)oxy)-7-methylquinoxalin-5-yl)ethyl)amino)benzoate (38.0 g, 74.8 mmol, 65.2% yield, 95.0% purity) as a yellow solid. LCMS [M+23]⁺=505.2.

¹H NMR (400 MHz, CDCl₃) δ=8.38 (d, J=6.0 Hz, 1H), 7.94 (dd, J=1.6, 8.0 Hz, 1H), 7.70 (s, 1H), 7.69 (s, 1H), 7.52 (d, J=8.8 Hz, 2H), 7.13-7.04 (m, 1H), 6.94-6.86 (m, 2H), 6.56 (t, J=7.2 Hz, 1H), 6.24 (d, J=8.4 Hz, 1H), 5.68-5.57 (m, 3H), 3.94 (s, 3H), 3.79 (s, 3H), 2.47 (s, 3H), 1.66 (d, J=6.8 Hz, 3H).

Step O: To a solution of methyl (R)-2-((1-(2-cyano-3-((4-methoxybenzyl)oxy)-7-methylquinoxalin-5-yl)ethyl)amino)benzoate (38.0 g, 74.8 mmol, 1.00 eq.) in dichloromethane (380 mL) was added 2,2,2-trifluoroacetic acid (127 g, 1.12 mol, 83.0 mL, 14.9 eq.) at 0° C., and the mixture was left to stir at 0° C. for 1 hour. The mixture was then poured into sodium hydrogen carbonate (saturated in water, 500 mL) slowly at 0° C., then the mixture was extracted with dichloromethane (400 mL×2). The combined organic layers were dried over anhydrous sodium sulfate, filtered, and concentrated under reduced pressure to give a residue. The residue was purified by reversed phase flash (column: Sfar C18 330 g D Duo 30 um; mobile phase: phase A: 0.225% formic acid in water, phase B: acetonitrile; gradient: 45%-80% phase B) to give methyl (R)-2-((1-(2-cyano-3-hydroxy-7-methylquinoxalin-5-yl) ethyl)amino)benzoate (22.6 g, 60.1 mmol, 80.4% yield, 96.4% purity) as a yellow solid. LCMS [M+1]⁺=363.0.

¹H NMR (400 MHz, DMSO-d₆) δ=12.89 (br s, 1H), 8.18 (br d, J=5.6 Hz, 1H), 7.81 (dd, J=1.6, 8.0 Hz, 1H), 7.52 (s, 1H), 7.44 (d, J=1.6 Hz, 1H), 7.24-7.14 (m, 1H), 6.56 (t, J=7.6 Hz, 1H), 6.31 (d, J=8.4 Hz, 1H), 5.35 (quin, J=6.0 Hz, 1H), 3.86 (s, 3H), 2.29 (s, 3H), 1.46 (d, J=6.4 Hz, 3H).

### Intermediate B

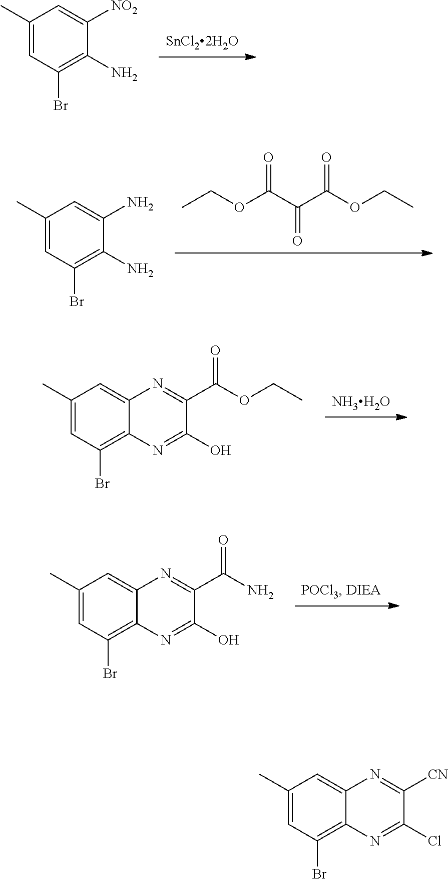

Step A: To a solution of 2-bromo-4-methyl-6-nitroaniline (30.0 g, 130 mmol, 1.00 eq.) in ethyl acetate (300 mL) was added tin (II) chloride dihydrate (147 g, 649 mmol, 5.00 eq.), and the mixture was left to stir at 80° C. for 3 hours. The reaction mixture was then cooled to 25° C., and the pH of the mixture was adjusted with sodium bicarbonate (saturated solution in water) to pH˜8. The mixture was then filtered, and the filter cake was washed with ethyl acetate (300 mL×3). The filtrate was further diluted with water (300 mL) and extracted with ethyl acetate (300 mL), and the combined organic layers were washed with brine (500 mL), dried over sodium sulfate, filtered, and concentrated under reduced pressure to give a residue. The crude product 3-bromo-5-methylbenzene-1,2-diamine (22.5 g, 112 mmol, 86.2% yield) was obtained as a yellow solid and used without further purification. LCMS [M+H]⁺=201.1.

Step B: To a solution of 3-bromo-5-methylbenzene-1,2-diamine (22.5 g, 112 mmol, 1.00 eq.) in ethanol (300 mL) was added diethyl 2-oxomalonate (23.4 g, 134 mmol, 20.7 mL, 1.20 eq.), and the mixture was left to stir at 80° C. for 4 hours. The mixture was then cooled to 25° C., filtered, and the filter cake was dried under vacuum to give ethyl 5-bromo-3-hydroxy-7-methylquinoxaline-2-carboxylate (31.5 g, 92.9 mmol, 83.1% yield) as a yellow solid. LCMS [M+3]⁺=313.1.

Step C: To a solution of ethyl 5-bromo-3-hydroxy-7-methylquinoxaline-2-carboxylate (31.5 g, 101 mmol, 1.00 eq.) in methanol (300 mL) was added ammonium hydroxide (63.4 g, 506 mmol, 69.6 mL, 28.0% purity, 5.00 eq.), and the mixture was stirred at 25° C. for 12 hours. The mixture was then filtered, and the filter cake concentrated under reduced pressure to give 5-bromo-3-hydroxy-7-methylquinoxaline-2-carboxamide (26.4 g, crude) as a yellow solid. LCMS [M+3]⁺=284.0.

Step D: To a solution of 5-bromo-3-hydroxy-7-methylquinoxaline-2-carboxamide (1.00 g, 3.54 mmol, 1.00 eq.) in POCl₃ (10.0 mL) was added diisopropylethylamine (2.29 g, 17.7 mmol, 3.09 mL, 5.00 eq.) at 0° C., and the mixture was left to stir at 120° C. for 12 hours. The mixture was then cooled to 25° C., concentrated in vacuo, and the residue was diluted with ethyl acetate (50.0 mL×2) at 0° C. The pH of the mixture was then adjusted to pH=7 with sodium bicarbonate (saturated solution in water) slowly, and the organic phase was washed with brine (50.0 mL×2), dried over anhydrous sodium sulfate, filtered, and concentrated under reduced pressure to give a crude product. The residue was purified by column chromatography (SiO₂, petroleum ether/ethyl acetate=50/1 to 20/1) to give 5-bromo-3-chloro-7-methylquinoxaline-2-carbonitrile (1.40 g, 4.86 mmol, 68.5% yield, 98.0% purity) as a yellow solid. LCMS [M+3]⁺=284.1.

¹H NMR (400 MHz, DMSO-d₆) δ=8.37 (d, J=1.6 Hz, 1H), 8.30 (d, J=1.6 Hz, 1H), 8.06 (s, 1H), 7.96 (s, 1H), 2.61 (s, 3H).

### Intermediate B—Alternative Synthesis

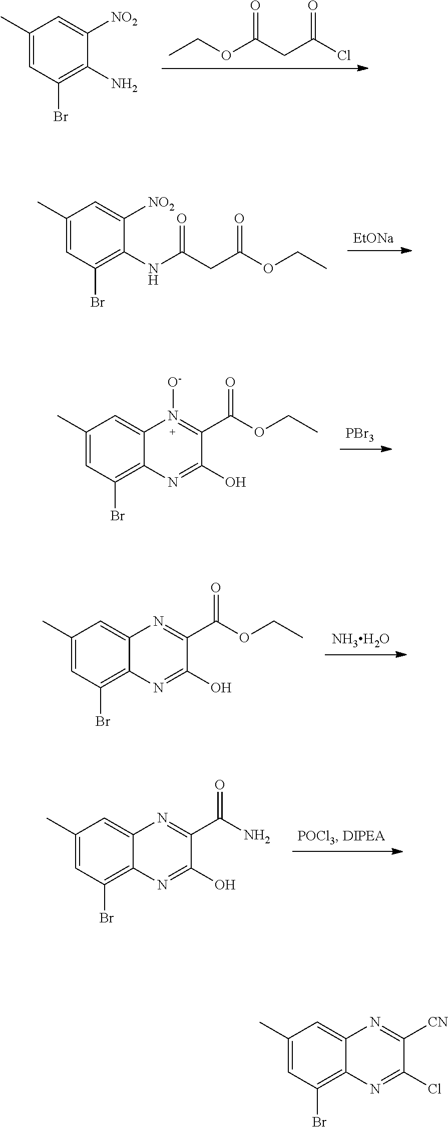

Step A: To A mixture of 2-bromo-4-methyl-6-nitroaniline (26.0 g, 113 mmol, 1.00 eq.) in toluene (250 mL) was added ethyl 3-chloro-3-oxopropanoate (18.6 g, 124 mmol, 15.6 mL, 1.10 eq.) at 0° C., then the reaction was heat to 90° C. and left to stir for 4 hours. The mixture was then cooled to 25° C., and diisopropyl ether (250 mL) was added, at which point a white precipitate was formed. The suspension was filtered, and the cake was collected and dried under reduced pressure to give ethyl 3-((2-bromo-4-methyl-6-nitrophenyl)amino)-3-oxopropanoate (33.0 g, 95.6 mmol, 85.0% yield) as a yellow solid. LCMS [M+3]⁺=347.2.

¹H NMR (400 MHz, DMSO-d₆) δ=10.33 (s, 1H), 7.93 (d, J=1.2 Hz, 1H), 7.81 (d, J=1.2 Hz, 1H), 4.11 (q, J=6.8 Hz, 2H), 3.46 (s, 2H), 2.40 (s, 3H), 1.21 (t, J=7.2 Hz, 3H).

Step B: To a solution of sodium ethoxide (81.3 g, 239 mmol, 75.0 mL, 20% purity, 2.50 eq.) in tetrahydrofuran (250 mL) and DMF (250 mL) was added a solution of ethyl 3-((2-bromo-4-methyl-6-nitrophenyl)amino)-3-oxopropanoate (33.0 g, 95.6 mmol, 1.00 eq.) in a mixed solvent of DMF (50.0 mL) and tetrahydrofuran (50.0 mL) dropwise at −30° C. over one hour. The mixture was then warmed to 0° C. and poured into cold water (80.0 mL), then 4N HCl (in water, 30.0 mL) was added. The pH of the mixture was adjusted to pH>7, and the mixture was extracted with ethyl acetate (100 mL×2), and the combined organic layers were washed with saturated brine (100 mL), dried over magnesium sulfate, filtered, and concentrated under reduced pressure to give a residue. The residue was triturated with diisopropylether (35.0 mL) to give the 5-bromo-2-(ethoxycarbonyl)-3-hydroxy-7-methylquinoxaline 1-oxide (25.0 g, 76.4 mmol, 79.9% yield) as a yellow solid which was used without further purification. LCMS [M+3]+=329.2.

Step C: To a solution of 5-bromo-2-(ethoxycarbonyl)-3-hydroxy-7-methylquinoxaline 1-oxide (23.0 g, 70.3 mmol, 1.00 eq.) in DMF (200 mL) was added PBr₃ (38.1 g, 141 mmol, 2.00 eq.) dropwise, then the solution was stirred at 65° C. for 1.5 hours. The mixture was then cooled to 25° C., poured into sodium bicarbonate solution (saturated in water, 400 mL), and extracted with ethyl acetate (400 mL×3). The combined organic layers were washed with saturated brine (100 mL×3), dried over magnesium sulfate, filtered, and concentrated to give a residue. The residue was purified by trituration with n-hexane (300 mL) to give ethyl 5-bromo-3-hydroxy-7-methylquinoxaline-2-carboxylate (19.0 g, 61.1 mmol, 86.9% yield) as yellow solid which was without further purification. LCMS [M+H]⁺=311.0.

¹H NMR (400 MHz, DMSO-d₆) δ=12.65-11.74 (m, 1H), 7.85 (s, 1H), 7.70 (s, 1H), 4.39 (q, J=7.2 Hz, 2H), 2.41 (s, 3H), 1.33 (t, J=7.2 Hz, 3H).

Step D: To a solution of ethyl 5-bromo-3-hydroxy-7-methylquinoxaline-2-carboxylate (5.70 g, 18.3 mmol, 1.00 eq.) in tetrahydrofuran (60.0 mL) was added ammonium hydroxide (6.88 g, 55.0 mmol, 7.56 mL, 28% purity, 3.00 eq.), and the mixture was stirred at 45° C. for 12 hours. Upon completion, the mixture was cooled to 25° C. and the mixture was concentrated under reduced pressure to give 5-bromo-3-hydroxy-7-methylquinoxaline-2-carboxamide (5.10 g, 18.1 mmol, 98.7% yield) as a yellow solid which was used without further purification. LCMS [M+3]⁺=283.7.

Step E: To a solution of 5-bromo-3-hydroxy-7-methylquinoxaline-2-carboxamide (4.0 g, 14.2 mmol, 1.00 eq.) in POCl₃ (40.0 mL) was added diisopropylethylamine (9.16 g, 70.9 mmol, 12.4 mL, 5.0 eq.) at 0° C. The mixture was then warmed to 120° C. and left to stir for 12 hours. The mixture was then cooled to 0° C., diluted with ethyl acetate (150 mL×2), and the solution was poured into aqueous sodium bicarbonate slowly, and the pH was adjusted to pH>7. The organic phase was washed with brine (100 mL×2), dried over anhydrous sodium sulfate, filtered, and concentrated under reduced pressure to give 5-bromo-3-chloro-7-methylquinoxaline-2-carbonitrile (4.42 g, crude) as brown solid which was used without further purification.

¹H NMR (400 MHz, CDCl₃) δ=8.05 (d, J=1.6 Hz, 1H), 7.84 (dd, J=0.8, 1.6 Hz, 1H), 2.56 (s, 3H).

### Intermediate C

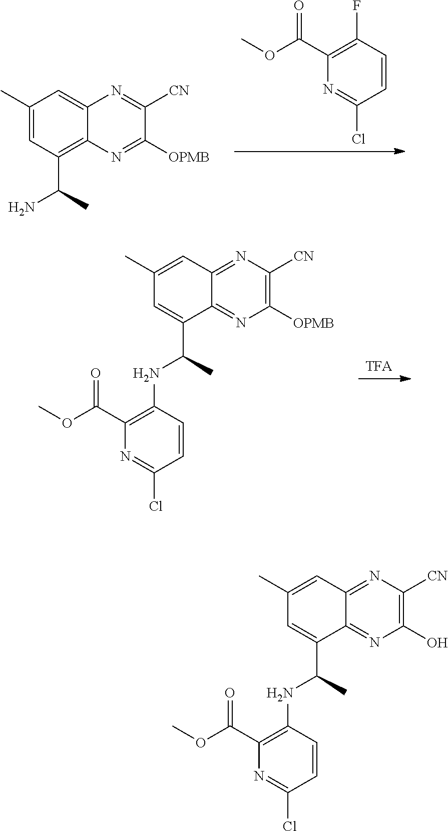

Step A: A mixture of (R)-5-(1-aminoethyl)-3-((4-methoxybenzyl)oxy)-7-methylquinoxaline-2-carbonitrile (2.67 g, 7.66 mmol, 1.00 eq.), methyl 6-chloro-3-fluoropicolinate (2.03 g, 10.7 mmol, 1.40 eq.), diisopropylethylamine (4.95 g, 38.3 mmol, 6.67 mL, 5.00 eq.) in N,N-dimethylformamide (30.0 mL) was degassed and purged with dinitrogen for 3 times, and then the mixture was stirred at 100° C. for 12 hours under dinitrogen atmosphere. After completion, the mixture was cooled to 20° C. The mixture was poured into water aqueous solution (10.0 mL) and extracted with ethyl acetate (30.0 mL×3). The combined organic layer was washed with brine (10.0 mL), dried over anhydrous sodium sulfate, then the mixture was filtered and the filtrate was concentrated. The residue was purified by column chromatography (SiO2, petroleum ether/ethyl acetate=1/0 to 3/1) to give methyl (R)-6-chloro-3-((1-(2-cyano-3-((4-methoxybenzyl)oxy)-7-methylquinoxalin-5-yl)ethyl)amino)picolinate (2.30 g, 4.00 mmol, 52.2% yield, 90% purity) as a yellow solid. LCMS [M+23]⁺=540.2.

¹H NMR (400 MHz, DMSO-d6) 5=8.40 (d, J=7.20 Hz, 1H), 7.88-7.76 (m, 2H), 7.52 (d, J=8.40 Hz, 2H), 7.28 (d, J=9.20 Hz, 1H), 7.05 (d, J=9.20 Hz, 1H), 6.98 (d, J=8.80 Hz, 2H), 5.74-5.48 (m, 3H), 3.87 (s, 3H), 3.76 (s, 3H), 2.48 (s, 3H), 1.67 (d, J=6.40 Hz, 3H)

Step B: To a solution of methyl (R)-6-chloro-3-((1-(2-cyano-3-((4-methoxybenzyl)oxy)-7-methylquinoxalin-5-yl)ethyl)amino)picolinate (1.30 g, 2.51 mmol, 1.00 eq.) in dichloromethane (10.0 mL) was added trifluoroacetic acid (4.61 g, 40.4 mmol, 3.00 mL, 16.1 eq.) at 0° C. The mixture was stirred at 25° C. for 1 hour. After completion, the mixture was concentrated to give methyl (R)-6-chloro-3-((1-(2-cyano-3-hydroxy-7-methylquinoxalin-5-yl)ethyl)amino)picolinate (2.50 g crude) as a yellow solid. LCMS [M+1]⁺=398.2.

### Example 1

### 2-((1-(2-cyano-7-methyl-3-(piperidin-1-yl)quinoxalin-5-yl)ethyl)amino)benzoic acid

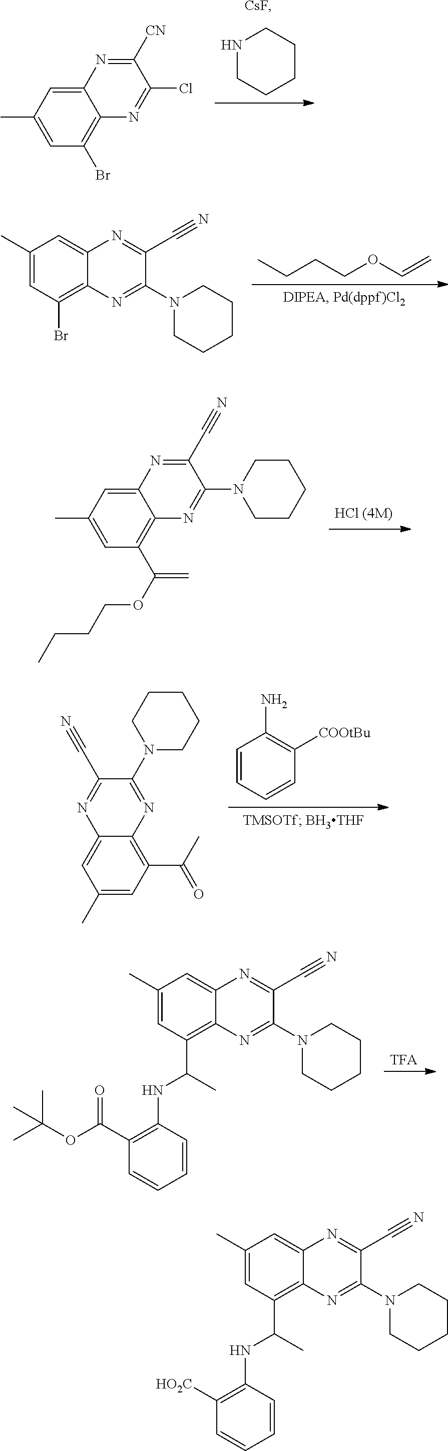

Step A: To a solution of 5-bromo-3-chloro-7-methylquinoxaline-2-carbonitrile (1.20 g, 4.25 mmol, 1.00 eq.) in dimethyl sulfoxide (12.0 mL) was added cesium fluoride (968 mg, 6.37 mmol, 235 μL, 1.50 eq.) and piperidine (723 mg, 8.49 mmol, 839 μL, 2.00 eq.). The mixture was stirred at 130° C. for 1 hour. After completion of the reaction, the mixture was cooled to 25° C., the mixture was diluted with water (20.0 mL), then extracted with ethyl acetate (20.0 mL×3). The combined the organic layers were dried over anhydrous sodium sulfate and concentrated under reduced pressure to give a residue. The residue was purified by silica gel chromatography (petroleum ether/ethyl acetate 50/1, 5/1) to give 5-bromo-7-methyl-3-(piperidin-1-yl)quinoxaline-2-carbonitrile (180 mg, 543 μmol, 12.8% yield) as a yellow solid. LCMS [M+H]⁺=331.2.

¹H NMR (400 MHz, DMSO-d₆) δ=7.80 (d, J=1.6 Hz, 1H), 7.52 (s, 1H), 3.70 (br d, J=5.2 Hz, 4H), 2.48 (br s, 3H), 1.68 (br s, 6H).

Step B: A mixture of 5-bromo-7-methyl-3-(piperidin-1-yl)quinoxaline-2-carbonitrile (300 mg, 906 μmol, 1.00 eq.), n-butyl vinyl ether (363 mg, 3.62 mmol, 466 μL, 4.00 eq.), Pd(dppf)Cl₂ (66.3 mg, 90.6 μmol, 0.10 eq.), and diisopropylethylamine (351 mg, 2.72 mmol, 473 μL, 3.00 eq.) in n-butyl alcohol (6.00 mL) was degassed and purged with nitrogen 3 times, and then the mixture was stirred at 120° C. for 8 hours under nitrogen atmosphere. After completion of the reaction, the mixture was added water (5.00 mL) and extracted with ethyl acetate (5.00 mL×3). Combined the organic layers were dried over anhydrous sodium sulfate and concentrated in vacuum to give 5-(1-butoxyvinyl)-7-methyl-3-(piperidin-1-yl)quinoxaline-2-carbonitrile (300 mg, crude) as a black solid. LCMS [M+H]⁺=351.2.

Step C: To a solution of 5-(1-butoxyvinyl)-7-methyl-3-(piperidin-1-yl)quinoxaline-2-carbonitrile (300 mg, 856 μmol, 1.00 eq.) in tetrahydrofuran (3.00 mL) was added hydrochloric acid (4.00 M in dioxane, 1.07 mL, 5.00 eq.). The mixture was stirred at 25° C. for 2 hours. The mixture was added water (20.00 mL) and extracted with ethyl acetate (20.00 mL×3). Combined the organic layers were dried over anhydrous sodium sulfate and concentrated in vacuum. The residue was purified by silica gel chromatography (petroleum ether/ethyl acetate 50/1, 1/1) to give 5-acetyl-7-methyl-3-(piperidin-1-yl)quinoxaline-2-carbonitrile (140 mg, 441 μmol, 51.7% yield, 92.8% purity) as a yellow solid. LCMS [M+H]⁺=294.9.

Step D: To a solution of 5-acetyl-7-methyl-3-(piperidin-1-yl)quinoxaline-2-carbonitrile (80.0 mg, 272 μmol, 1.00 eq.) in N,N-dimethylformamide (0.50 mL) under nitrogen was added tert-butyl 2-aminobenzoate (63.0 mg, 326 μmol, 59.0 μL, 1.20 eq.) and trimethylsilyl trifluoromethanesulfonate (151 mg, 679 μmol, 123 μL, 2.50 eq.) at 0° C., Then a solution of borane tetrahydrofuran complex (1.00 M, 272 μL, 1.00 eq.) was added. The mixture was stirred at 0° C. for 3 hours. The mixture was quenched with methanol (3.00 mL) at 0° C. Next, the mixture was diluted with ethyl acetate 5.00 mL and washed with brine (3.00 mL×3), the separated organic phase was dried over anhydrous sodium sulfate, filtered and concentrated under reduced pressure to give a residue. The mixture was purified by prep-TLC (petroleum ether/ethyl acetate 10:1) to give tert-butyl 2-((1-(2-cyano-7-methyl-3-(piperidin-1-yl)quinoxalin-5-yl)ethyl)amino)benzoate (40.0 mg, 84.8 μmol, 31.2% yield) as a yellow solid. LCMS [M+H]⁺=472.5.

Step E: To a solution of tert-butyl 2-((1-(2-cyano-7-methyl-3-(piperidin-1-yl)quinoxalin-5-yl)ethyl)amino)benzoate (40.0 mg, 84.8 μmol, 1.00 eq.) was added trifluoroacetic acid (29.0 mg, 254 μmol, 18.9 μL, 3.00 eq.). The mixture was stirred at 25° C. for 2 hours. The reaction was concentrated under reduced pressure to remove trifluoroacetic acid and the residue was neutralized with ammonium hydroxide. The mixture was purified by prep-HPLC (column: YMC-Actus Triart C18 150×30 mm×7 um; mobile phase: mobile phase A: 0.225% formic acid in water, mobile phase B: acetonitrile; gradient B %: 80%-100%, 10 min) to give 2-((1-(2-cyano-7-methyl-3-(piperidin-1-yl)quinoxalin-5-yl)ethyl)amino)benzoic acid (11.2 mg, 26.0 μmol, 30.7% yield, 96.6% purity) as a yellow solid. LCMS [M+H]⁺=279.2.

¹H NMR (400 MHz, DMSO-d₆) δ=8.47 (br d, J=3.2 Hz, 1H), 7.78 (dd, J=1.6, 8.0 Hz, 1H), 7.63 (s, 1H), 7.61 (d, J=1.6 Hz, 1H), 7.19-7.13 (m, 1H), 6.50 (t, J=7.6 Hz, 1H), 6.42 (d, J=8.4 Hz, 1H), 5.51-5.45 (m, 1H), 3.71 (br d, J=6.0 Hz, 4H), 2.41 (s, 3H), 1.78-1.67 (m, 6H), 1.61 (d, J=6.8 Hz, 3H).

### Example 2

### 2-((1-(2-cyano-3-(isoindolin-2-yl)-7-methylquinoxalin-5-yl)ethyl)amino)benzoic acid

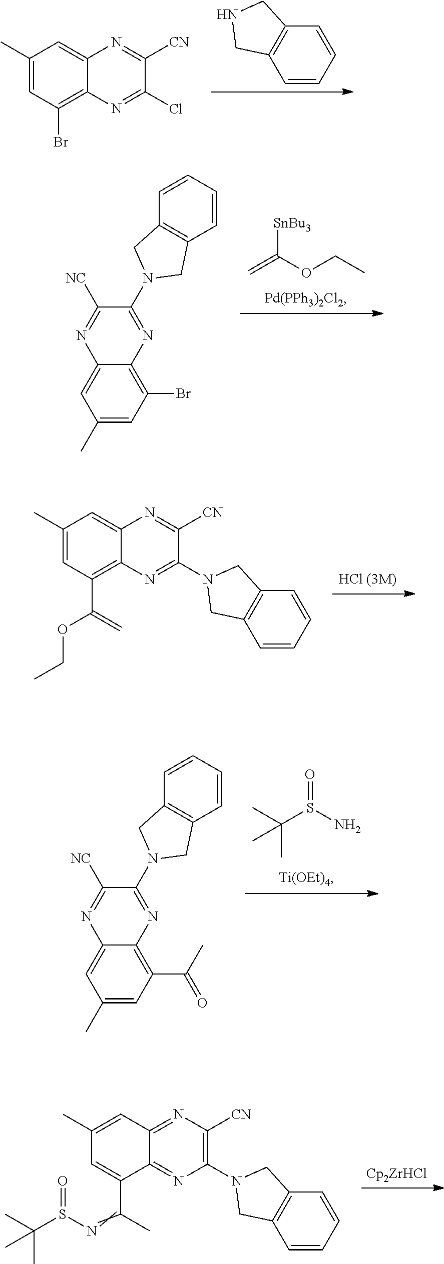

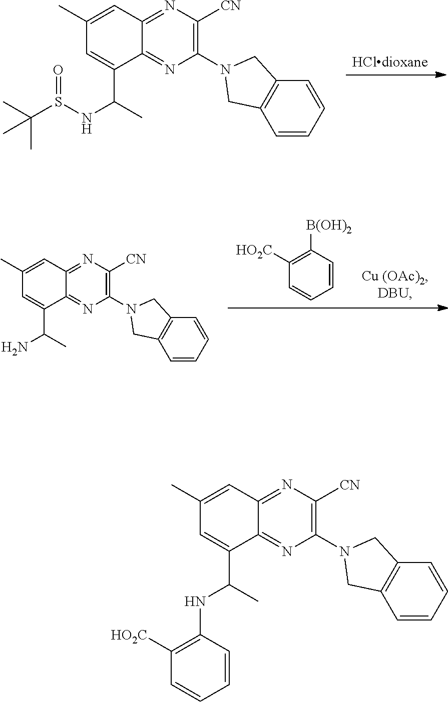 [SMILES: Cc1cc(C(C)NS(=O)C(C)(C)C)c2nc(N3Cc4ccccc4C3)c(C#N)nc2c1 ; "HCl·dioxane" ; Cc1cc(C(C)N)c2nc(N3Cc4ccccc4C3)c(C#N)nc2c1 ; O=C(O)c1ccccc1B(O)O ; "Cu (OAc)2,DBU," ; Cc1cc(C(C)Nc2ccccc2C(=O)O)c2nc(N3Cc4ccccc4C3)c(C#N)nc2c1]

Step A: To a solution of isoindoline (2.80 g, 23.5 mmol, 2.67 mL, 4.74 eq.) in dimethyl sulfoxide (2.50 mL) was added 5-bromo-3-chloro-7-methylquinoxaline-2-carbonitrile (1.40 g, 4.96 mmol, 1.00 eq.) portion-wise. The mixture was stirred at 15° C. for 1 hour. Upon completion, the reaction mixture was poured into water (20.0 mL), during this period, yellow precipitate was formed, the suspension was filtered, the cake was collected and dried under reduced pressure to give the crude product. The crude product was triturated with a mixture solvent of petroleum ether (3.00 mL) and ethyl acetate (9.00 mL), then filtered and collected the cake 5-bromo-3-(isoindolin-2-yl)-7-methylquinoxaline-2-carbonitrile (1.60 g, 4.38 mmol, 88.4% yield) as a yellow solid which was use directly. LCMS [M+3]⁺=367.1.

¹H NMR (400 MHz, CDCl₃) δ=7.81 (d, J=1.6 Hz, 1H), 7.59 (s, 1H), 7.42-7.32 (m, 2H), 7.31-7.26 (m, 2H), 7.16-7.01 (m, 2H), 5.35-5.07 (m, 4H), 2.42 (s, 3H).

Step B: A mixture of 5-bromo-3-(isoindolin-2-yl)-7-methylquinoxaline-2-carbonitrile (1.60 g, 4.38 mmol, 1.00 eq.), tributyl(1-ethoxyvinyl)tin (2.21 g, 6.12 mmol, 2.07 mL, 1.40 eq.), Pd(PPh₃)₂Cl₂ (307 mg, 438 μmol, 0.10 eq.) in dioxane (15.0 mL) was stirred at 100° C. for 4 hours under a nitrogen atmosphere. After completion of the reaction, the reaction mixture was cooled to 20° C., then the reaction mixture was quenched by addition KF solution 40 mL at 25° C. and stirred for 1 hour, the solution was extracted with ethyl acetate 150 mL (50.0 mL×3). The combined organic layers were washed with water 60.0 mL (30.0 mL×2), dried over, filtered and concentrated under reduced pressure to give 5-(1-ethoxyvinyl)-3-(isoindolin-2-yl)-7-methylquinoxaline-2-carbonitrile (1.50 g, crude) as a yellow solid. LCMS [M+1]⁺=357.2.

Step C: To a solution of 5-(1-ethoxyvinyl)-3-(isoindolin-2-yl)-7-methylquinoxaline-2-carbonitrile (1.50 g, 4.21 mmol, 1.00 eq.) in acetonitrile (20.0 mL) at 10° C. was added HCl (3 M in water, 1.40 mL, 1.00 eq.). The mixture was stirred at 10° C. for 1 hour. Upon completion, the mixture was quenched by saturated sodium bicarbonate solution to pH=7 slowly. The suspension was precipitated with ethyl acetate (8.00 mL) and the yellow cake was collected by filtration, the solid was washed with ethyl acetate (2.00 mL×2), then dried to give 5-acetyl-3-(isoindolin-2-yl)-7-methylquinoxaline-2-carbonitrile (1.10 g, 3.35 mmol, 79.6% yield) as a yellow solid which was used directly. LCMS [M+1]⁺=329.1.

Step D: To a solution 5-acetyl-3-(isoindolin-2-yl)-7-methylquinoxaline-2-carbonitrile (250 mg, 761 μmol, 1.00 eq.), 2-methylpropane-2-sulfinamide (185 mg, 1.52 mmol, 2.00 eq.) in a mixture of tetrahydrofuran (2.10 mL) and 1,2-dimethoxyethane (0.36 mL) was added titanium (IV) ethoxide (521 mg, 2.28 mmol, 474 μL, 3.00 eq.). The mixture was stirred at 80° C. for 16 hours. Upon completion, the mixture was cooled to 25° C., water (0.50 mL) was added to the reaction, then the suspension was filtered, the solid was washed by ethyl acetate 160 mL (40.0 mL×4, the combined organics were washed with brine 15.0 mL (5.0 mL×3), dried over anhydrous sodium sulfate, filtered and concentrated under reduced pressure to give N-(1-(2-cyano-3-(isoindolin-2-yl)-7-methylquinoxalin-5-yl)ethylidene)-2-methylpropane-2-sulfinamide (320 mg, crude) as yellow solid which was used directly. LCMS [M+1]⁺=432.1.

Step E: To a solution of N-(1-(2-cyano-3-(isoindolin-2-yl)-7-methylquinoxalin-5-yl)ethylidene)-2-methylpropane-2-sulfinamide (320 mg, 742 μmol, 1.00 eq.) in dichloromethane (3.00 mL) at 0° C. was added zirconocene hydrochloride (792 mg, 2.97 mmol, 4.00 eq.) portion-wise. The mixture was stirred at 0° C., and then warmed to 25° C. stirred for 3 hours. Upon completion, the mixture was quenched by water 2.00 mL and stirred for 20 min. The suspension was filtered and extracted with dichloromethane (30.0 mL×3). The organic phase was separated, washed with brine 4.00 mL, dried over anhydrous sodium sulfate, filtered and concentrated under reduced pressure to give a residue. The residue was purified by column chromatography (SiO₂, petroleum ether/ethyl acetate=5/1 to 1/1) to give N-(1-(2-cyano-3-(isoindolin-2-yl)-7-methylquinoxalin-5-yl)ethyl)-2-methylpropane-2-sulfinamide (110 mg, 233 μmol, 31.4% yield, 91.8% purity) as a yellow solid. LCMS [M+1]⁺=434.1.

¹H NMR (400 MHz, CDCl₃) δ=7.64 (s, 1H), 7.55 (d, J=1.6 Hz, 1H), 7.49-7.41 (m, 2H), 7.41-7.34 (m, 2H), 5.42-5.27 (m, 4H), 5.23 (quin, J=6.4 Hz, 1H), 4.54 (br d, J=6.0 Hz, 1H), 2.53 (s, 3H), 1.72 (d, J=6.8 Hz, 3H), 1.26 (s, 9H).

Step F: To a solution of N-(1-(2-cyano-3-(isoindolin-2-yl)-7-methylquinoxalin-5-yl)ethyl)-2-methylpropane-2-sulfinamide (110 mg, 233 μmol, 1.00 eq.) in acetonitrile (1.50 mL) was added HCl (4 M in dioxane, 116 μL, 2.00 eq.). The mixture was stirred at 0° C. for 30 minutes. Upon completion, the reaction mixture was directly concentrated to give a crude product. The crude product was triturated with ethyl acetate (1.00 mL) and dried to give 5-(1-aminoethyl)-3-(isoindolin-2-yl)-7-methylquinoxaline-2-carbonitrile (80.0 mg, 219 μmol, 93.9% yield, hydrochloride salt) as a green solid which was used directly. LCMS [M+1]⁺=330.1.

Step G: A mixture of 5-(1-aminoethyl)-3-(isoindolin-2-yl)-7-methylquinoxaline-2-carbonitrile (80.0 mg, 219 μmol, 1.00 eq., hydrochloride salt), 2-carboxyphenylboronic acid (109 mg, 656 μmol, 3.00 eq.), 1,8-diazabicyclo[5.4.0]undec-7-ene (166 mg, 1.09 mmol, 165 μL, 5.00 eq.), copper (II) acetate (59.6 mg, 328 μmol, 1.50 eq.) and 4 Å molecular sieves (30.0 mg) in dimethylformamide (1.50 mL) was stirred at 25° C. for 16 hours. Upon completion, the mixture was filtered, the filtrate was diluted water (2.00 mL), ethyl acetate (5.00 mL) was added and the layers were separated. The aqueous phase was extracted with ethyl acetate (15.0 mL×3). Combined extracts were washed with brine (3.00 mL), dried over anhydrous sodium sulfate, filtered, and concentrated under reduced pressure to give a residue. The residue was purified by prep-HPLC (column: Phenomenex Luna C18 150×25 mm×10 um; mobile phase: mobile phase: phase A: 0.225% formic acid in water, phase B: acetonitrile; gradient: 68%-98% phase B) to give 2-((1-(2-cyano-3-(isoindolin-2-yl)-7-methylquinoxalin-5-yl)ethyl)amino)benzoic acid (12.5 mg, 25.5 μmol, 11.7% yield, 91.6% purity) as a yellow solid. LCMS [M+1]⁺=450.2.

¹H NMR (400 MHz, DMSO-d₆) δ=12.95-12.19 (m, 1H), 8.69-8.38 (m, 1H), 7.78 (br d, J=7.2 Hz, 1H), 7.60 (s, 2H), 7.50 (br dd, J=3.2, 5.2 Hz, 2H), 7.36 (br dd, J=3.2, 5.6 Hz, 2H), 7.18 (br t, J=7.6 Hz, 1H), 6.60-6.44 (m, 2H), 5.64-5.48 (m, 1H), 5.36-5.23 (m, 4H), 2.40 (s, 3H), 1.68 (br d, J=6.8 Hz, 3H).

### Examples 3 and 4

### (R)-2-((1-(2-cyano-3-(4,4-difluoropiperidin-1-yl)-7-methylquinoxalin-5-yl)ethyl)amino)benzoate and (S)-2-((1-(2-cyano-3-(4,4-difluoropiperidin-1-yl)-7-methylquinoxalin-5-yl)ethyl)amino)benzoate

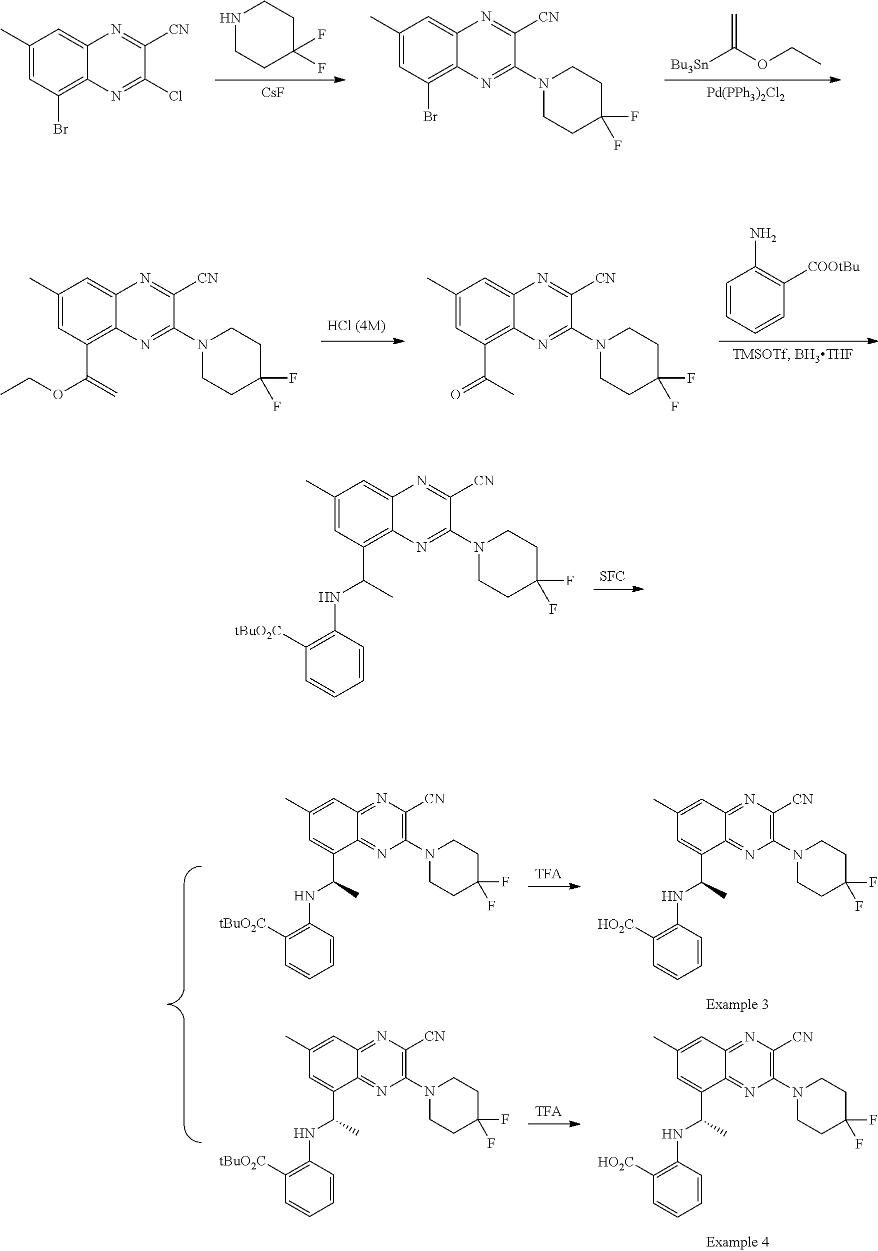

Step A: To a solution of 5-bromo-3-chloro-7-methylquinoxaline-2-carbonitrile (4.42 g, 15.6 mmol, 1.00 eq.) in dimethyl sulfoxide (30.0 mL) was added CsF (3.56 g, 23.5 mmol, 866 μL, 1.50 eq.) and 4,4-difluoropiperidine (3.22 g, 26.6 mmol, 1.70 eq.). The mixture was stirred at 130° C. for 2 hours. Upon completion, the mixture was cooled to 20° C., the reaction mixture was added to water (80.0 mL) slowly, during this period, yellow precipitate was formed, the suspension was filtered, the cake was collected and dried under reduced pressure to give the crude product. The crude product was purified by column chromatography (SiO₂, petroleum ether/ethyl acetate=20/1 to 10/1) to give 5-bromo-3-(4,4-difluoropiperidin-1-yl)-7-methylquinoxaline-2-carbonitrile (4.87 g, 13.26 mmol, 84.8% yield) as a yellow solid. LCMS [M+1]⁺=366.9.

¹H NMR (400 MHz, CDCl₃) δ=7.85 (d, J=1.6 Hz, 1H), 7.63 (d, J=0.8 Hz, 1H), 3.98-3.80 (m, 4H), 2.45 (s, 3H), 2.26-2.08 (m, 4H).

Step B: A mixture of 5-bromo-3-(4,4-difluoropiperidin-1-yl)-7-methylquinoxaline-2-carbonitrile (4.80 g, 13.07 mmol, 1 eq), tributyl(1-ethoxyvinyl)tin (6.10 g, 16.9 mmol, 5.71 mL, 1.29 eq.), Pd(PPh₃)₂Cl₂ (918 mg, 1.31 mmol, 0.10 eq.) in dioxane (50.0 mL) was stirred at 100° C. for 2 hours under a nitrogen atmosphere. After completion, the reaction mixture was warmed to 0° C., then the reaction mixture was quenched by addition KF solution 100 mL at 25° C. and stirred for 1 hour, the solution was extracted with ethyl acetate (80.0 mL×2). The combined organic layers were washed with water (30.0 mL×2), dried over, filtered and concentrated under reduced pressure to give 3-(4,4-difluoropiperidin-1-yl)-5-(1-ethoxyvinyl)-7-methylquinoxaline-2-carbonitrile (4.68 g, 13.06 mmol, crude) as a white solid which was used next step directly. LCMS [M+1]⁺=359.1.

Step C: To a solution of 3-(4,4-difluoropiperidin-1-yl)-5-(1-ethoxyvinyl)-7-methylquinoxaline-2-carbonitrile (4.65 g, 13.0 mmol, 1.00 eq.) in acetonitrile (40.0 mL) at 10° C. was added HCl (3 M in water, 5.19 mL, 1.20 eq.). The mixture was stirred at 10° C. for 1 hours. Upon completion, the mixture was quenched by saturated sodium bicarbonate solution to pH=7 and then extracted with ethyl acetate (100 mL×3). The combined organic phase was washed with saturated brine (40.0 mL×2), dried over anhydrous sodium sulfate, filtered and concentrated to give a crude product, the crude product was triturated with a mixed solvent of petroleum ether (30.0 mL) and ethyl acetate (10.0 mL) to give 5-acetyl-3-(4,4-difluoropiperidin-1-yl)-7-methylquinoxaline-2-carbonitrile (3.90 g, 11.8 mmol, 91.0% yield) as a yellow solid which used next step directly.

¹H NMR (400 MHz, CDCl₃) δ=7.96 (d, J=2.0 Hz, 1H), 7.84 (dd, J=0.8, 2.0 Hz, 1H), 3.88-3.70 (m, 4H), 2.80 (s, 3H), 2.50 (s, 3H), 2.28-2.10 (m, 4H)

Step D: A dry 100 mL three neck flask with magnetic stirring bar was charged consecutively with 5-acetyl-3-(4,4-difluoropiperidin-1-yl)-7-methylquinoxaline-2-carbonitrile (500 mg, 1.51 mmol, 1.00 eq.), tert-butyl 2-aminobenzoate (322 mg, 1.67 mmol, 304 μL, 1.10 eq.), N,N-dimethylformamide (5.00 mL), and trimethylsilyl trifluoromethanesulfonate (841 mg, 3.78 mmol, 684 μL, 2.50 eq.) under a nitrogen atmosphere. The reaction mixture was cooled to 0° C., then a solution of borane tetrahydrofuran complex (1 M in tetrahydrofuran, 1.82 mL, 1.20 eq.) was added slowly with a syringe over a period of 10-20 min. the reaction mixture was kept stirring at 0° C. and stirred for 12 hours. Upon completion, the mixture was quenched water (5.0 mL) and the mixture was stirred for 20 min, the solution was extracted with ethyl acetate (20.0 mL), followed by saturated sodium carbonate solution (12.0 mL), The two phase mixture was kept stirring until the gas evolution had ceased. The phases were separated, and the aqueous layer was extracted with ethyl acetate (20 mL×3). The combined organic layers were dried over anhydrous sodium sulfate, and the solvents were removed under reduced pressure to give a residue. The residue was purified by column chromatography (SiO₂, petroleum ether/ethyl acetate=1/0 to 10/1) to give the crude product. The crude product was further purified by prep-TLC (SiO₂, petroleum ether/ethyl acetate=1/0 to 10/1) give tert-butyl 2-((1-(2-cyano-3-(4,4-difluoropiperidin-1-yl)-7-methylquinoxalin-5-yl)ethyl)amino)benzoate (90.0 mg, 177 μmol, 11.7% yield) as a yellow solid. LCMS [M+1]⁺=508.1

### Separation of tert-butyl (R)-2-((1-(2-cyano-3-(4,4-difluoropiperidin-1-yl)-7-methylquinoxalin-5-yl)ethyl)amino)benzoate and tert-butyl (S)-2-((1-(2-cyano-3-(4,4-difluoropiperidin-1-yl)-7-methylquinoxalin-5-yl)ethyl)amino)benzoate

85 mg of tert-butyl 2-((1-(2-cyano-3-(4,4-difluoropiperidin-1-yl)-7-methylquinoxalin-5-yl)ethyl)amino)benzoate was purified by prep-HPLC (column: DAICEL CHIRALPAK IG (250 mm×50 mm, 10 um); mobile phase: phase A: CO₂, phase B: 0.1% ammonium hydroxide in i-PrOH; phase B %: 10%, isocratic elution mode) to give tert-butyl (R)-2-((1-(2-cyano-3-(4,4-difluoropiperidin-1-yl)-7-methylquinoxalin-5-yl)ethyl)amino)benzoate (40.0 mg, 78.8 μmol, 47.1% yield) (first eluting isomer) as a yellow oil. Tert-butyl (S)-2-((1-(2-cyano-3-(4,4-difluoropiperidin-1-yl)-7-methylquinoxalin-5-yl)ethyl)amino)benzoate (35.0 mg, 69.0 μmol, 41.2% yield) (second eluting isomer) as a yellow oil.

Step E: To a solution of tert-butyl (R)-2-((1-(2-cyano-3-(4,4-difluoropiperidin-1-yl)-7-methylquinoxalin-5-yl)ethyl)amino)benzoate (40.0 mg, 78.8 μmol, 1.00 eq.) in dichloromethane (1.00 mL) was added trifluoroacetic acid (0.50 mL). The mixture was stirred at 25° C. for 8 hours. After completion of the reaction, the mixture was adjusted to pH=7 with aqueous sodium hydroxide, then concentrated to give a residue. The residue was purified by prep-HPLC (column: Phenomenex luna C18 150×25 mm×10 um; mobile phase: mobile phase A: 0.225% formic acid in water, phase B: acetonitrile; gradient: 60%-90% B) to give (R)-2-((1-(2-cyano-3-(4,4-difluoropiperidin-1-yl)-7-methylquinoxalin-5-yl)ethyl)amino)benzoic acid (8.10 mg, 17.48 μmol, 22.2% yield, 97.4% purity) as a yellow solid. LCMS [M+H]⁺=452.2.

¹H NMR (400 MHz, CD₃OD) δ=7.90 (d, J=8.0 Hz, 1H), 7.72-7.62 (m, 2H), 7.18-7.07 (m, 1H), 6.52 (t, J=7.4 Hz, 1H), 6.41 (d, J=8.4 Hz, 1H), 5.60 (q, J=6.4 Hz, 1H), 3.93 (br t, J=5.6 Hz, 4H), 2.47 (s, 3H), 2.33-2.19 (m, 4H), 1.69 (d, J=6.8 Hz, 3H).

Step F: To a solution of tert-butyl (S)-2-((1-(2-cyano-3-(4,4-difluoropiperidin-1-yl)-7-methylquinoxalin-5-yl)ethyl)amino)benzoate (35.0 mg, 69.0 μmol, 1.00 eq.) in dichloromethane (1.00 mL) was added trifluoroacetic acid (0.50 mL). The mixture was stirred at 25° C. for 8 hours. After completion of the reaction, the mixture was adjusted to pH=7 with sodium hydroxide, then concentrated to give a residue. The residue was purified by prep-HPLC (column: Phenomenex luna C18 150×25 mm×10 um; mobile phase: phase A: 0.225% formic acid in water, phase B: acetonitrile; gradient: 65%-95% phase B) to give (S)-2-((1-(2-cyano-3-(4,4-difluoropiperidin-1-yl)-7-methylquinoxalin-5-yl)ethyl)amino)benzoic acid (15.5 mg, 34.16 μmol, 49.5% yield, 99.5% purity) as a yellow solid. LCMS [M+H]⁺=452.3.

¹H NMR (400 MHz, CD₃OD) δ=7.79 (dd, J=1.4, 7.9 Hz, 1H), 7.57-7.55 (m, 2H), 7.08-6.94 (m, 1H), 6.41 (t, J=7.6 Hz, 1H), 6.30 (d, J=8.4 Hz, 1H), 5.49 (q, J=6.4 Hz, 1H), 3.81 (t, J=5.6 Hz, 4H), 2.36 (s, 3H), 2.23-2.08 (m, 4H), 1.57 (d, J=6.8 Hz, 3H)

### Example 5

### (R)-6-chloro-3-((1-(2-cyano-3-(4,4-difluoropiperidin-1-yl)-7-methylquinoxalin-5-yl)ethyl)amino)picolinic acid

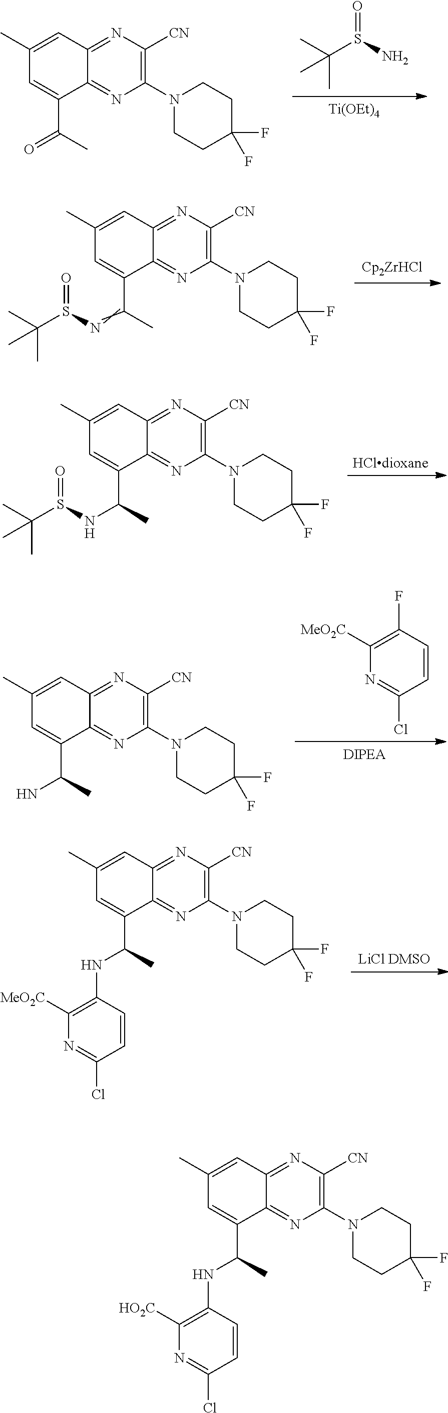

Step A: To a solution of 5-acetyl-3-(4,4-difluoropiperidin-1-yl)-7-methylquinoxaline-2-carbonitrile (1.50 g, 4.54 mmol, 1.00 eq.), (R)-2-methylpropane-2-sulfinamide (1.10 g, 9.08 mmol, 2.00 eq.) in a mixture of tetrahydrofuran (15.0 mL) and 1,2-dimethoxyethane (3.00 mL) was added titanium (IV) ethoxide (3.11 g, 13.6 mmol, 2.82 mL, 3.00 eq.). The mixture was stirred at 75° C. for 16 hours. Upon completion of the reaction, the mixture was cooled to 25° C., the reaction mixture was added water (2.00 mL) and stirred for 0.5 hour at 25° C. The reaction mixture was filtered, the cake was washed with ethyl acetate (50.0 mL×4), the combined organic layers were washed with brine 60.0 mL (20.0 mL×3), dried over anhydrous sodium sulfate, filtered and concentrated under reduced pressure to give (R)—N-(1-(2-cyano-3-(4,4-difluoropiperidin-1-yl)-7-methylquinoxalin-5-yl)ethylidene)-2-methylpropane-2-sulfinamide (1.80 g, 4.15 mmol, 91.4% yield) as yellow oil which was used directly.

Step B: To a solution of (R)—N-(1-(2-cyano-3-(4,4-difluoropiperidin-1-yl)-7-methylquinoxalin-5-yl)ethylidene)-2-methylpropane-2-sulfinamide, in dichloromethane (30.0 mL) was added zirconocene hydrochloride (2.94 g, 11.0 mmol, 4.0 eq.) portion-wise. The mixture was stirred at 25° C. stirred for 12 hours. Upon completion, the mixture was quenched by water (10 mL) and stirred for 20 min. Next, the suspension was filtered and diluted with dichloromethane (30.0 mL×3). The organic phase was separated, washed with brine 20.0 mL, dried over anhydrous sodium sulfate, filtered and concentrated under reduced pressure to give a residue. The residue was purified by column chromatography (SiO₂, petroleum ether/ethyl acetate=10/1 to 1/1) to give (R)—N—((R)-1-(2-cyano-3-(4,4-difluoropiperidin-1-yl)-7-methylquinoxalin-5-yl)ethyl)-2-methylpropane-2-sulfinamide (1.24 g, 2.57 mmol, 67.6% yield, 90.3% purity) as a yellow solid. LCMS [M+1]⁺=436.1.

¹H NMR (400 MHz, CDCl₃) δ=7.62 (d, J=0.8 Hz, 1H), 7.53 (d, J=1.6 Hz, 1H), 5.13 (quin, J=6.8 Hz, 1H), 4.21 (br d, J=6.8 Hz, 1H), 3.77 (br t, J=5.6 Hz, 4H), 2.47 (s, 3H), 2.30-2.09 (m, 4H), 1.57 (d, J=6.8 Hz, 3H), 1.23-1.10 (m, 9H).

Step C: To a solution of (R)—N—((R)-1-(2-cyano-3-(4,4-difluoropiperidin-1-yl)-7-methylquinoxalin-5-yl)ethyl)-2-methylpropane-2-sulfinamide (330 mg, 722.84 μmol, 1.00 eq.) in acetonitrile (3.00 mL) at 0° C. was added HCl (4 M in dioxane, 361.42 μL, 2.00 eq.) dropwise. The mixture was stirred at 0° C. for 1 hour. The reaction mixture was directly concentrated to give a crude product. The crude product was triturated with ethyl acetate (1.00 mL), collected the cake and dried to give (R)-5-(1-aminoethyl)-3-(4,4-difluoropiperidin-1-yl)-7-methylquinoxaline-2-carbonitrile (205 mg, 557.33 μmol, 77.1% yield, hydrochloride salt) as a yellow solid. LCMS [M+1]⁺=332.1.

Step D: A mixture of (R)-5-(1-aminoethyl)-3-(4,4-difluoropiperidin-1-yl)-7-methylquinoxaline-2-carbonitrile (195 mg, 530 μmol, 1.00 eq, hydrochloride salt), methyl 6-chloro-3-fluoro-pyridine-2-carboxylate (241 mg, 1.27 mmol, 2.40 eq.), diisopropylethylamine (480 mg, 3.71 mmol, 646 μL, 7.00 eq.) in dimethylformamide (1.50 mL) was stirred at 80° C. for 9 hours. Upon completion, the mixture was cooled to 25° C., the reaction mixture was diluted with water (20.0 mL) and extracted with ethyl acetate (40.0 mL×2). The combined organic layers were washed with saturate sodium bicarbonate solution (20.0 mL), dried over anhydrous sodium sulfate and concentrated under reduced pressure to give methyl (R)-6-chloro-3-((1-(2-cyano-3-(4,4-difluoropiperidin-1-yl)-7-methylquinoxalin-5-yl)ethyl)amino)picolinate (280 mg, 430 μmol, 81.2% yield, 77% purity) as yellow oil which was used directly. LCMS [M+1]⁺=501.2.

Step E: A mixture of methyl (R)-6-chloro-3-((1-(2-cyano-3-(4,4-difluoropiperidin-1-yl)-7-methylquinoxalin-5-yl)ethyl)amino)picolinate (90.0 mg, 138 μmol, 1.00 eq.), lithium chloride (58.7 mg, 1.38 mmol, 28.4 μL, 10.0 eq.) in dimethyl sulfoxide (0.50 mL) was stirred at 130° C. for 3 hours. Upon completion, the mixture was cooled to 25° C., the reaction mixture was filtered. The filtrate was purified by prep-HPLC (column: YMC-Actus Triart C18 150×30 mm×7 um; mobile phase: phase A: 0.225% formic acid in water, phase B: acetonitrile; gradient 63%-93% phase B) to give (R)-6-chloro-3-((1-(2-cyano-3-(4,4-difluoropiperidin-1-yl)-7-methylquinoxalin-5-yl)ethyl)amino)picolinic acid (22.2 mg, 45.2 μmol, 32.7% yield, 99.2% purity) as a yellow solid. LCMS [M+23]⁺=509.1

¹H NMR (400 MHz, CDCl₃) δ=11.07-10.33 (m, 1H), 8.38 (br d, J=6.4 Hz, 1H), 7.70 (s, 1H), 7.58 (d, J=1.6 Hz, 1H), 7.13 (d, J=8.8 Hz, 1H), 6.77 (d, J=8.8 Hz, 1H), 5.51 (quin, J=6.4 Hz, 1H), 3.99-3.83 (m, 4H), 2.50 (s, 3H), 2.39-2.17 (m, 4H), 1.73 (d, J=6.8 Hz, 3H).

### Example 6

### (R)-2-((1-(2-cyano-3-(isoindolin-2-yl)-7-methylquinoxalin-5-yl)ethyl)amino)benzoic acid

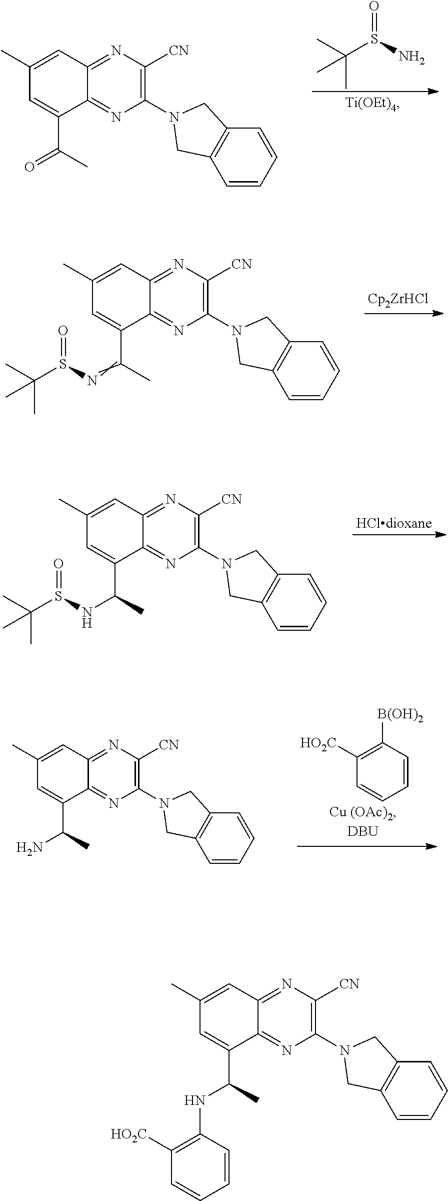

Step A: To a solution of 5-acetyl-3-(isoindolin-2-yl)-7-methylquinoxaline-2-carbonitrile (500 mg, 1.52 mmol, 1.00 eq.), (R)-2-methylpropane-2-sulfinamide (369 mg, 3.05 mmol, 2.00 eq.) in a mixture of tetrahydrofuran (4.20 mL) and 1,2-dimethoxyethane (0.72 mL) was added tetraethoxytitanium (1.04 g, 4.57 mmol, 947 μL, 3.00 eq.). The mixture was stirred at 80° C. stirred for 16 hours. Upon completion, the mixture was cooled to 25° C., the reaction mixture was quenched with water (0.50 mL), then the suspension was filtered, the cake was washed by ethyl acetate (20.0 mL×4), the combined organics were washed with brine (2.00 mL×3), dried over anhydrous sodium sulfate, filtered and concentrated under reduced pressure to give (R)—N-(1-(2-cyano-3-(isoindolin-2-yl)-7-methylquinoxalin-5-yl)ethylidene)-2-methylpropane-2-sulfinamide (680 mg, crude) as yellow solid which was used directly. LCMS [M+1]⁺=432.2

Step B: To a solution of (R)—N-(1-(2-cyano-3-(isoindolin-2-yl)-7-methylquinoxalin-5-yl)ethylidene)-2-methylpropane-2-sulfinamide (600 mg, 1.39 mmol, 1.00 eq.) in dichloromethane (9.00 mL) at 0° C. was added zirconocene hydrochloride (1.48 g, 5.56 mmol, 4.00 eq.) portion-wise. The mixture was stirred at 0° C., and then warmed to 25° C. stirred for 3 hours. Upon completion, the mixture was quenched by water 5 mL and stirred for 20 min. The suspension filtered and extracted with dichloromethane (30.0 mL×3). The organic phase was separated, washed with brine 10.0 mL, dried over anhydrous sodium sulfate, filtered and concentrated under reduced pressure to give a residue. The residue was purified by column chromatography (SiO₂, petroleum ether/ethyl acetate=5/1 to 1/1) to give (R)—N—((R)-1-(2-cyano-3-(isoindolin-2-yl)-7-methylquinoxalin-5-yl)ethyl)-2-methylpropane-2-sulfinamide (286 mg, 641 μmol, 46.1% yield, 97.2% purity) as a yellow solid.

¹H NMR (400 MHz, CDCl₃) δ=7.66 (s, 1H), 7.57 (d, J=1.6 Hz, 1H), 7.51-7.43 (m, 2H), 7.43-7.37 (m, 2H), 5.43-5.29 (m, 4H), 5.24 (quin, J=6.4 Hz, 1H), 4.56 (br d, J=6.4 Hz, 1H), 2.67-2.50 (m, 1H), 2.54 (s, 2H), 1.73 (d, J=6.8 Hz, 3H), 1.28 (s, 9H).

Step C: To a solution of (R)—N—((R)-1-(2-cyano-3-(isoindolin-2-yl)-7-methylquinoxalin-5-yl)ethyl)-2-methylpropane-2-sulfinamide (130 mg, 291 μmol, 1.00 eq.) in acetonitrile (1.50 mL) at 0° C. was added HCl (4 M in dioxane, 146 μL, 2.00 eq.), the mixture was stirred at 0° C. for 1 hour. Upon completion, the reaction mixture was directly concentrated to give a crude product. The crude product was triturated with ethyl acetate (1.00 mL) and dried to give (R)-5-(1-aminoethyl)-3-(isoindolin-2-yl)-7-methylquinoxaline-2-carbonitrile (70.0 mg, 191 μmol, 65.7% yield, hydrochloride salt) as a yellow solid. LCMS [M+1]⁺=330.2.

Step D: A mixture of (R)-5-(1-aminoethyl)-3-(isoindolin-2-yl)-7-methylquinoxaline-2-carbonitrile (70.0 mg, 191 μmol, 1.00 eq., hydrochloride salt), 2-carboxyphenylboronic acid (95.3 mg, 574 μmol, 3.00 eq.), 1,8-diazabicyclo[5.4.0]undec-7-ene (146 mg, 957 μmol, 144 μL, 5.00 eq.), copper (II) acetate (52.1 mg, 287 μmol, 1.50 eq.) and 4 Å molecular sieve (50.0 mg, 13.7 μmol) in N,N-dimethylformamide (1.00 mL) was stirred at 25° C. for 16 hours. Upon completion, the mixture was filtered and the filtrate was diluted water (2.00 mL). Next, ethyl acetate (5.00 mL) was added and the layers were separated. The aqueous phase was extracted with ethyl acetate (15.0 mL×3). The combined organics were washed with brine (3.00 mL), dried over anhydrous sodium sulfate, filtered, and concentrated under reduced pressure to give a residue. The residue was purified by prep-HPLC (column: Phenomenex Luna C18 150×25 mm×10 um; mobile phase: phase A: 0.225% formic acid in water, phase B: acetonitrile; gradient: 68%-98% phase B) to give (R)-2-((1-(2-cyano-3-(isoindolin-2-yl)-7-methylquinoxalin-5-yl)ethyl)amino)benzoic acid (17.0 mg, 37.7 μmol, 19.7% yield, 99.6% purity) as a yellow solid. LCMS [M+1]⁺=450.2.

¹H NMR (400 MHz, CDCl₃) δ=8.45-8.11 (m, 1H), 7.99 (dd, J=1.2, 8.0 Hz, 1H), 7.61 (d, J=8.0 Hz, 2H), 7.50-7.41 (m, 2H), 7.40-7.34 (m, 2H), 7.25-7.12 (m, 1H), 6.59 (t, J=7.2 Hz, 1H), 6.43 (d, J=8.4 Hz, 1H), 5.72-5.54 (m, 1H), 5.36 (s, 4H), 2.46 (s, 3H), 1.76 (d, J=6.8 Hz, 3H).

### Example 7

### 6-chloro-3-(((1R)-1-(2-cyano-3-(6,6-difluoro-3-azabicyclo[3.1.1]heptan-3-yl)-7-methylquinoxalin-5-yl)ethyl)amino)picolinic acid

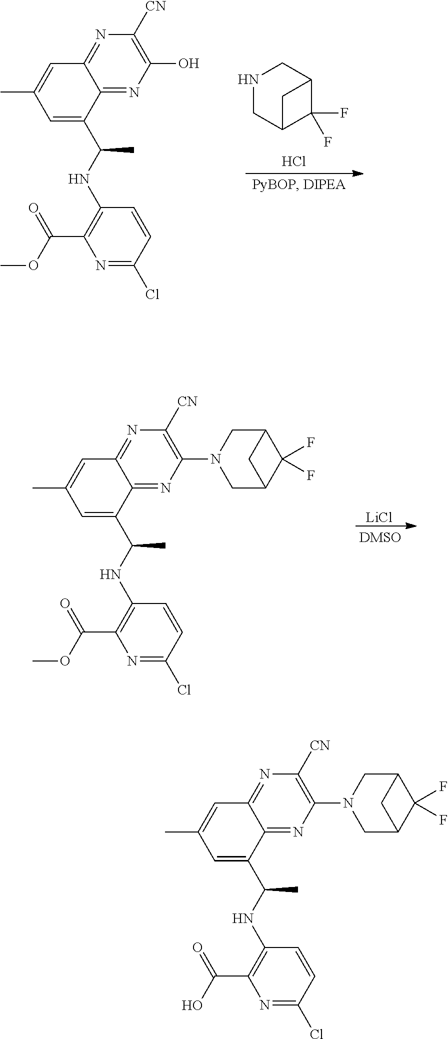 [SMILES: COC(=O)c1nc(Cl)ccc1N[C@H](C)c1cc(C)cc2nc(C#N)c(O)nc12 ; FC1(F)C2CNCC1C2 ; Cl ; COC(=O)c1nc(Cl)ccc1N[C@H](C)c1cc(C)cc2nc(C#N)c(N3CC4CC(C3)C4(F)F)nc12 ; "LiCl" ; Cc1cc([C@@H](C)Nc2ccc(Cl)nc2C(=O)O)c2nc(N3CC4CC(C3)C4(F)F)c(C#N)nc2c1]

Step A: To a solution of methyl (R)-6-chloro-3-((1-(2-cyano-3-hydroxy-7-methylquinoxalin-5-yl)ethyl)amino)picolinate (100 mg, 251 μmol, 1.00 eq.) in dimethyl sulfoxide (1.00 mL) was added benzotriazole-1-yl-oxy-tris-pyrrolidino-phosphonium hexafluorophosphate (196 mg, 377 μmol, 1.50 eq.), 6,6-difluoro-3-azabicyclo[3.1.1]heptane hydrochloride (77.7 mg, 377 μmol, 1.50 eq.) and diisopropylethylamine (162 mg, 1.26 mmol, 219 μL, 5.00 eq.). The mixture was stirred at 20° C. for 1 hour. After completion, the mixture was poured into saturated water aqueous solution (1.00 mL) and extracted with ethyl acetate (3×5.00 mL). The combined organics were washed with brine (1.00 mL), the organic layers were collected and concentrated to give a residue. The residue was purified by prep-TLC (SiO₂, petroleum ether/ethyl acetate=3/1) to give methyl 6-chloro-3-(((1R)-1-(2-cyano-3-(6,6-difluoro-3-azabicyclo[3.1.1]heptan-3-yl)-7-methylquinoxalin-5-yl)ethyl)amino)picolinate (50.0 mg, 87.7 μmol, 34.9% yield, 75% purity) as a white solid. LCMS [M+1]⁺=513.3.

Step B: To a solution of methyl 6-chloro-3-(((1R)-1-(2-cyano-3-(6,6-difluoro-3-azabicyclo[3.1.1]heptan-3-yl)-7-methylquinoxalin-5-yl)ethyl)amino)picolinate (40.0 mg, 82.9 μmol, 1.00 eq.) in dimethyl sulfoxide (0.50 mL) was added lithium chloride (35.1 mg, 829 μmol, 10.0 eq.). The reaction was stirred at 170° C. for 1 hour. After completion, the mixture was cooled to 20° C. The mixture was filtered. The filtrate was purified by prep-HPLC (column: Waters Xbridge 150×25 mm×5 um; mobile phase: phase A: water (10 mmol/L NH₄HCO₃), phase B: acetonitrile; gradient: 10%-40% phase B) and further purified by prep-HPLC (column: Phenomenex luna C18 150×25 mm×10 um; mobile phase: phase A: 0.225% formic acid in water, phase B: acetonitrile; gradient: 15%-45% phase B) to give 6-chloro-3-(((1R)-1-(2-cyano-3-(6,6-difluoro-3-azabicyclo[3.1.1]heptan-3-yl)-7-methylquinoxalin-5-yl)ethyl)amino)picolinic acid (5.36 mg, 11.0 μmol, 13.3% yield, 96.1% purity) as a yellow solid. LCMS [M+23]⁺=521.3.

¹H NMR (400 MHz, DMSO-d₆) δ=8.66-8.31 (m, 1H), 7.63 (t, J=6.40 Hz, 2H), 7.29 (d, J=8.80 Hz, 1H), 7.17-6.91 (m, 1H), 5.54-5.37 (m, 1H), 4.53 (br dd, J=4.00, 12.4 Hz, 1H), 4.36-4.20 (m, 1H), 3.80-3.64 (m, 2H), 3.63-3.50 (m, 1H), 3.12-2.98 (m, 1H), 2.96-2.78 (m, 1H), 2.52 (br s, 1H), 2.41 (d, J=2.00 Hz, 3H), 1.66 (t, J=7.20 Hz, 3H)

¹⁹F NMR (400 MHz, DMSO-d₆) δ=−92.47, −93.84, −94.83.

### Examples 8 and 9

### 6-chloro-3-(((R)-1-(2-cyano-3-((1 S,4S)-5,5-difluoro-2-azabicyclo[2.2.1]heptan-2-yl)-7-methylquinoxalin-5-yl)ethyl)amino)picolinic acid (Example 8) and 6-chloro-3-(((R)-1-(2-cyano-3-((1R,4R)-5,5-difluoro-2-azabicyclo[2.2.1]heptan-2-yl)-7-methylquinoxalin-5-yl)ethyl)amino)picolinic acid (Example 9)

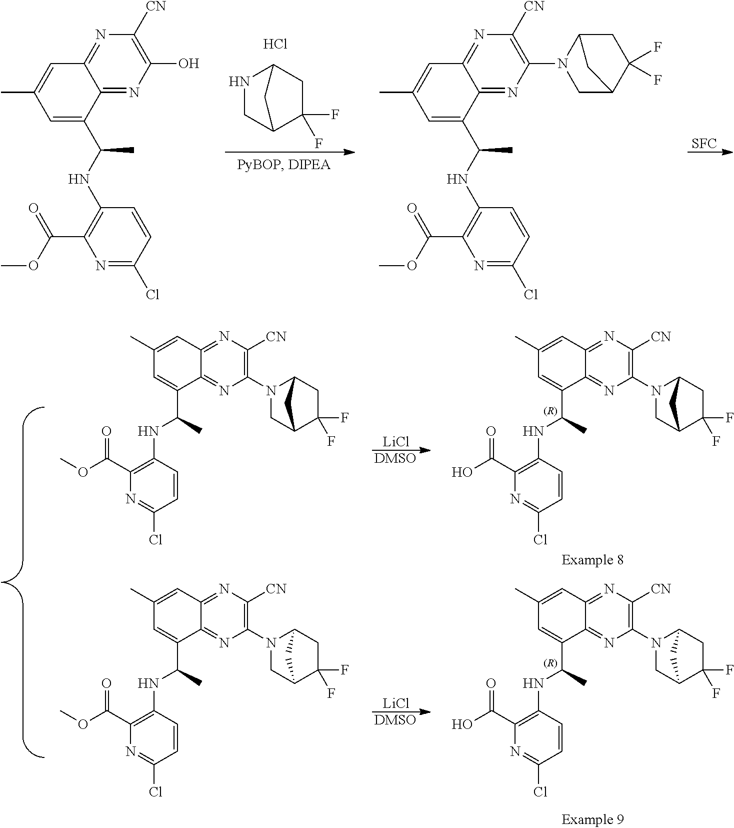

Step A: To a solution of methyl (R)-6-chloro-3-((1-(2-cyano-3-hydroxy-7-methylquinoxalin-5-yl)ethyl)amino)picolinate (150 mg, 377 μmol, 1.00 eq.) in N,N-dimethylformamide (1.50 mL) was added diisopropylethylamine (244 mg, 1.89 mmol, 328 μL, 5.00 eq.) and benzotriazole-1-yl-oxy-tris-pyrrolidino-phosphonium hexafluorophosphate (294 mg, 565 μmol, 1.50 eq.). The reaction was stirred at 20° C. for 15 minutes. And then 5,5-difluoro-2-azabicyclo[2.2.1]heptane hydrochloride (95.9 mg, 565 μmol, 1.50 eq.) was added to the above mixture and the mixture was stirred at 20° C. for 1 hour. After completion, the reaction mixture was diluted with water (5.00 mL) and extracted with ethyl acetate (3×5.00 mL). The organic layers were combined and concentrated to give a residue. The residue was purified by prep-TLC (petroleum ether:ethyl acetate=3/1) to give methyl 6-chloro-3-(((1R)-1-(2-cyano-3-(5,5-difluoro-2-azabicyclo[2.2.1]heptan-2-yl)-7-methylquinoxalin-5-yl)ethyl)amino)picolinate (180 mg, 316 μmol, 83.8% yield, 90% purity) as a yellow solid. LCMS [M+1]⁺=513.1.

Step B The mixture of methyl 6-chloro-3-(((1R)-1-(2-cyano-3-(5,5-difluoro-2-azabicyclo[2.2.1]heptan-2-yl)-7-methylquinoxalin-5-yl)ethyl)amino)picolinate (180 mg, 316 μmol, 1.00 eq.) was purified by Supercritical Fluid Chromatography (SFC) (column: DAICEL CHIRALCEL OX (250 mm×30 mm, 10 um); mobile phase: phase A: CO₂, phase B: 0.1% ammonium hydroxide in MeOH; 40% phase B isocratic elution mode). The desired fractions of the first peak were collected and concentrated to give the methyl ester of Example 8 (30.0 mg, 42.7 μmol, 12.2% yield, 73% purity) as a yellow solid (first eluting isomer). LCMS [M+1]⁺=513.2. The desired fractions of the second peak were collected and concentrated to give the methyl ester of Example 9 (30.0 mg, 40.4 μmol, 11.5% yield, 69% purity) as a yellow solid (second eluting isomer). LCMS [M+1]⁺=513.2.

### Saponification of the Methyl Esters of Examples 8 and 9

To a solution of the methyl ester of Example 8 (25.0 mg, 48.7 μmol, 1.00 eq.) in dimethyl sulfoxide (0.50 mL) was added lithium chloride (20.7 mg, 487 μmol, 10.0 eq.). The mixture was stirred at 130° C. for 4 hours. After completion, the mixture was cooled to 20° C. and filtered. The filtrate was purified by prep-HPLC (column: Welch Xtimate C18 150*25 mm*5 um; mobile phase: phase A: 0.225% formic acid in water, phase B: acetonitrile; gradient: 55%-85% phase B) to give Example 8 (9.45 mg, 18.7 μmol, 38.3% yield, 98.5% purity) as a yellow solid. LCMS [M+23]⁺=521.2.

¹H NMR (400 MHz, CDCl₃-d) δ=10.71 (s, 1H), 8.37 (br d, J=6.0 Hz, 1H), 7.61 (s, 1H), 7.49 (d, J=1.2 Hz, 1H), 7.13 (d, J=8.8 Hz, 1H), 6.78 (d, J=8.8 Hz, 1H), 5.44-5.34 (m, 1H), 5.10 (br s, 1H), 4.14-4.05 (m, 1H), 3.92 (d, J=10.4 Hz, 1H), 3.11-3.02 (m, 1H), 2.44 (s, 3H), 2.41-2.27 (m, 2H), 2.20-2.07 (m, 2H), 1.70 (d, J=6.8 Hz, 3H).

¹⁹F NMR (400 MHz, CDCl₃-d) δ=−89.30, −89.91, −108.36, −108.96.

To a solution of the methyl ester of Example 9 (30.0 mg, 58.5 μmol, 1.00 eq.) in dimethyl sulfoxide (0.50 mL) was added lithium chloride (24.8 mg, 585 μmol, 10.0 eq.). The reaction was stirred at 130° C. for 4 hours. After completion, the mixture was cooled to 20° C. and filtered. The filtrate was purified by prep-HPLC (column: Welch Xtimate C18 150×25 mm×5 um; mobile phase: phase A: 0.225% formic acid in water, phase B: acetonitrile, gradient: 55%-85% phase B) to give Example 9 (6.22 mg, 12.3 μmol, 21.0% yield, 98.6% purity) as a yellow solid. LCMS [M+23]⁺=521.2.

¹H NMR (400 MHz, CDCl₃-d) δ=8.40-8.31 (d, J=5.6 Hz, 1H), 7.61 (s, 1H), 7.50 (s, 1H), 7.10 (d, J=8.8 Hz, 1H), 6.71 (d, J=8.8 Hz, 1H), 5.47-5.37 (m, 1H), 5.06 (br s, 1H), 4.15-4.07 (m, 1H), 3.91 (d, J=10.4 Hz, 1H), 3.11-3.03 (m, 1H), 2.44 (s, 3H), 2.41-2.27 (m, 2H), 2.20-2.07 (m, 2H), 1.69 (d, J=6.8 Hz, 3H).

¹⁹F NMR (400 MHz, CDCl₃-d) δ=−89.32, −89.92, −108.53, −109.13.

### Examples 10 and 11

### 6-chloro-3-(((R)-1-(2-cyano-3-((1R,4S)-6,6-difluoro-2-azabicyclo[2.2.1]heptan-2-yl)-7-methylquinoxalin-5-yl)ethyl)amino)picolinic acid (Example 11) and 6-chloro-3-(((R)-1-(2-cyano-3-((1 S,4R)-6,6-difluoro-2-azabicyclo[2.2.1]heptan-2-yl)-7-methylquinoxalin-5-yl)ethyl)amino)picolinic acid (Example 10)

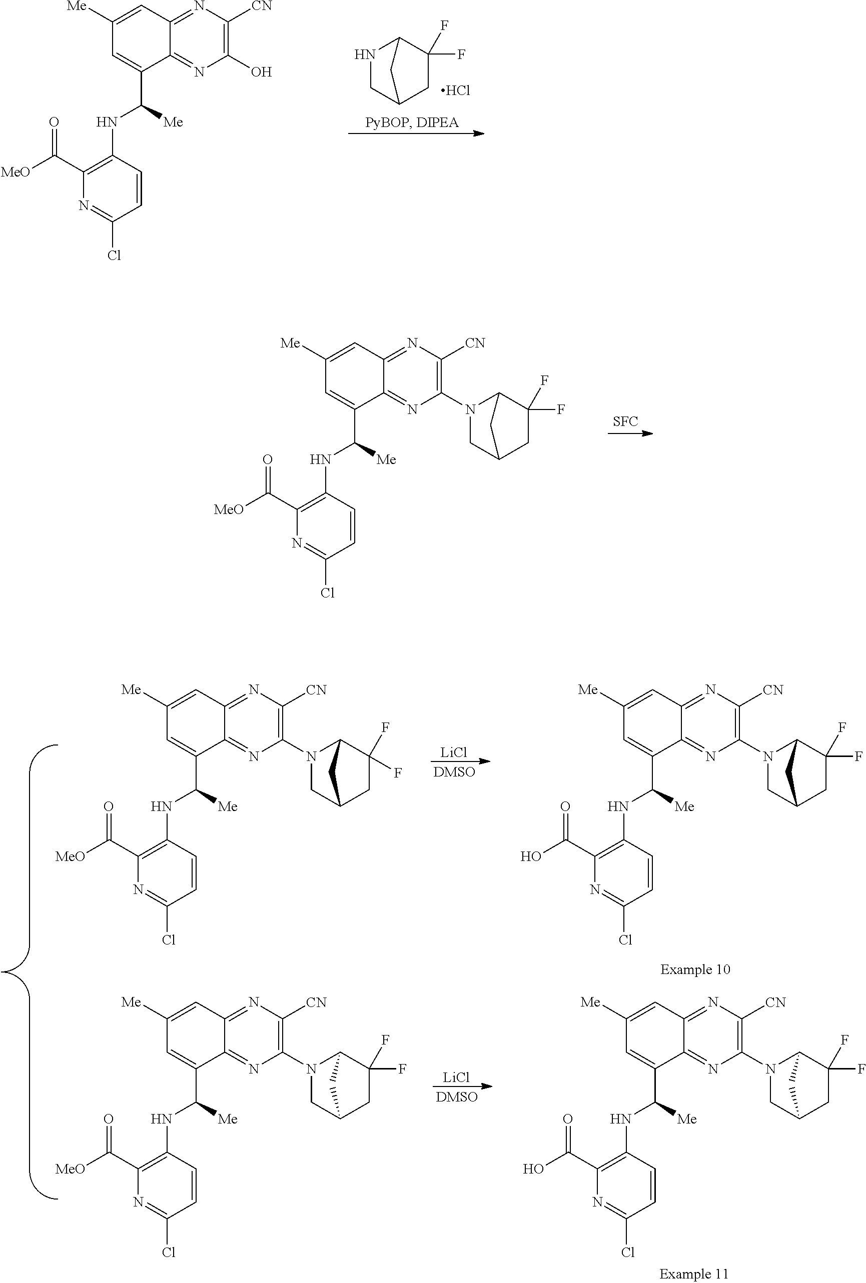

Step A: To a solution of methyl (R)-6-chloro-3-((1-(2-cyano-3-hydroxy-7-methylquinoxalin-5-yl)ethyl)amino)picolinate (200 mg, 502 μmol, 1.00 eq.) in N,N-dimethylformamide (4.00 mL) were added benzotriazole-1-yl-oxy-tris-pyrrolidino-phosphonium hexafluorophosphate (392 mg, 754 μmol, 1.50 eq.) and diisopropylethylamine (162 mg, 1.26 mmol, 219 μL, 5.00 eq.). The reaction was stirred at 25° C. for 30 minutes. Then, to the above mixture was added 6,6-difluoro-2-azabicyclo[2.2.1]heptane hydrochloride (128 mg, 754 μmol, 1.50 eq.) at 25° C., the reaction was stirred at 25° C. for 48 hours. After completion, the mixture was diluted with water (10.0 mL) and extracted with ethyl acetate (3×20.0 mL). The combined organic layers were washed with brine (3×10.0 mL), dried over anhydrous sodium sulfate, filtered and concentrated under reduced pressure to give a residue. The residue was purified by prep-TLC (SiO₂, petroleum ether:ethyl acetate=2:1) to give methyl 6-chloro-3-(((1R)-1-(2-cyano-3-(6,6-difluoro-2-azabicyclo[2.2.1]heptan-2-yl)-7-methylquinoxalin-5-yl)ethyl)amino)picolinate (90.0 mg, 175 μmol, 34.9% yield) as a yellow solid.

Step B: 6-chloro-3-(((1R)-1-(2-cyano-3-(6,6-difluoro-2-azabicyclo[2.2.1]heptan-2-yl)-7-methylquinoxalin-5-yl)ethyl)amino)picolinate (90.0 mg, 175 μmol) was purified by SFC (column: DAICEL CHIRALCEL OX (250 mm×30 mm, 10 um); mobile phase: phase A: CO₂, phase B: ammonium hydroxide in MeOH; phase B %: 40%, isocratic elution mode) to give methyl ester of Example 10 (25 mg, 48.7 μmol, 27.8% yield) as a yellow solid (first eluting isomer) and the methyl ester of Example 11 (40.0 mg, 78.0 μmol, 44.4% yield) as a yellow solid was isolated as the second eluting isomer.

### Saponification of the Methyl Esters of Examples 10 and 11

To a solution of methyl ester of Example 10 (25.0 mg, 48.7 μmol, 1.00 eq.) in dimethyl sulfoxide (0.20 mL) was added lithium chloride (10.3 mg, 244 μmol, 5.00 eq.). The reaction was stirred at 130° C. for 1 hour. After completion, the mixture was cooled to 20° C. and purified by prep-HPLC (column: Waters Xbridge 150×25 mmx 5 um; mobile phase: phase A: water (10 mmol/L NH₄HCO₃), phase B: acetonitrile; gradient: 22%-52% phase B) to give Example 10 (9.22 mg, 17.9 μmol, 36.6% yield, 96.6% purity) as a yellow solid. LCMS [M+23]⁺=521.2.

¹H NMR (400 MHz, DMSO-d₆) δ=9.24 (br s, 1H), 7.61 (s, 1H), 7.58 (s, 1H), 7.06 (br d, J=8.4 Hz, 1H), 6.84 (br d, J=6.8 Hz, 1H), 5.39 (s, 2H), 4.01-3.93 (m, 1H), 3.75 (d, J=9.6 Hz, 1H), 2.90 (br s, 1H), 2.40 (s, 3H), 2.30-2.17 (m, 1H), 2.12-1.90 (m, 3H), 1.61 (d, J=6.4 Hz, 3H).

¹⁹F NMR (377 MHz, DMSO-d₆) δ=−89.18, −89.76, −110.69, −111.27.

Step D: To a solution of methyl ester of Example 11 (40.0 mg, 78.0 μmol, 1.00 eq.) in dimethyl sulfoxide (0.40 mL) was added lithium chloride (16.5 mg, 390 μmol, 5.00 eq.). The reaction was stirred at 130° C. for 1 hour. After completion, the mixture was cooled to 20° C. and purified by prep-HPLC (column: Waters Xbridge 150×25 mm×5 um; mobile phase: phase A: water (10 mmol/L NH₄HCO₃), phase B: acetonitrile; gradient: 22%-52% phase B) to give Example 11 (7.38 mg, 14.51 μmol, 18.6% yield, 98.1% purity) as a yellow solid. LCMS [M+23]+=521.2.

¹H NMR (400 MHz, DMSO-d₆) δ=9.20 (br s, 1H), 7.57 (s, 1H), 7.51 (d, J=1.6 Hz, 1H), 7.08 (d, J=8.8 Hz, 1H), 6.71 (br d, J=8.8 Hz, 1H), 5.39 (br d, J=6.4 Hz, 1H), 5.33 (s, 1H), 4.00 (br d, J=8.0 Hz, 1H), 3.68 (d, J=9.6 Hz, 1H), 2.89 (br s, 1H), 2.37 (s, 3H), 2.30-2.16 (m, 1H), 2.11-1.88 (m, 3H), 1.58 (d, J=6.8 Hz, 3H).

¹⁹F NMR (400 MHz, DMSO-d₆) δ=−89.67, −90.26, −110.71, −111.28.

### Example 12

### (R)-6-chloro-3-((1-(2-cyano-3-(3,3-difluoropiperidin-1-yl)-7-methylquinoxalin-5-yl)ethyl)amino)picolinic acid

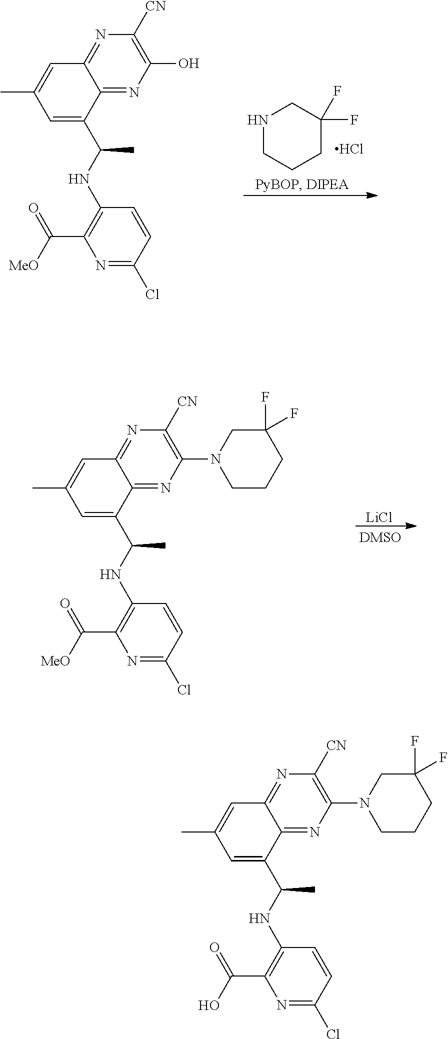 [SMILES: COC(=O)c1nc(Cl)ccc1N[C@H](C)c1cc(C)cc2nc(C#N)c(O)nc12 ; FC1(F)CCCNC1 ; Cl ; COC(=O)c1nc(Cl)ccc1N[C@H](C)c1cc(C)cc2nc(C#N)c(N3CCCC(F)(F)C3)nc12 ; "LiCl" ; Cc1cc([C@@H](C)Nc2ccc(Cl)nc2C(=O)O)c2nc(N3CCCC(F)(F)C3)c(C#N)nc2c1]

Step A: To a solution of methyl (R)-6-chloro-3-((1-(2-cyano-3-hydroxy-7-methylquinoxalin-5-yl)ethyl)amino)picolinate (90.0 mg, 226 μmol, 1.00 eq.) in N,N-dimethylformamide (0.20 mL) were added benzotriazole-1-yl-oxy-tris-pyrrolidino-phosphonium hexafluorophosphate (177 mg, 339 μmol, 1.50 eq.) and diisopropylethylamine (146 mg, 1.213 mmol, 197 μL, 5.00 eq.). The reaction was stirred at 25° C. for 30 minutes. Then, to above mixture was added 3,3-difluoropiperidine hydrochloride (41.1 mg, 261 μmol, 1.15 eq.) at 25° C. The reaction was stirred at 25° C. for 11.5 hours. After completion, the mixture was diluted with water (10.0 mL) and extracted with ethyl acetate (10.0 mL×3). The combined organics were washed with brine (10.0 mL×3), dried over anhydrous sodium sulfate, filtered and concentrated under reduced pressure to give a residue. The residue was purified by prep-TLC (SiO₂, petroleum ether:ethyl acetate=2:1) to give methyl (R)-6-chloro-3-((1-(2-cyano-3-(3,3-difluoropiperidin-1-yl)-7-methylquinoxalin-5-yl)ethyl)amino)picolinate (30.0 mg, 46.1 μmol, 20.4% yield) as a yellow solid. LCMS [M+1]⁺=501.1.

Step B: To a solution of methyl (R)-6-chloro-3-((1-(2-cyano-3-(3,3-difluoropiperidin-1-yl)-7-methylquinoxalin-5-yl)ethyl)amino)picolinate (20.0 mg, 39.9 μmol, 1.00 eq.) in dimethyl sulfoxide (0.20 mL) was added lithium chloride (8.46 mg, 200 μmol, 5.00 eq.). The reaction was stirred at 130° C. for 5 hours. After completion, the mixture was cooled to 20° C. and purified by prep-HPLC (column: Phenomenex Luna C18 150×25 mm×10 um; mobile phase: phase A: water (10 mmol/L NH₄HCO₃), phase B: acetonitrile; gradient: 26%-56% phase B) to give (R)-6-chloro-3-((1-(2-cyano-3-(3,3-difluoropiperidin-1-yl)-7-methylquinoxalin-5-yl)ethyl)amino)picolinic acid (12.7 mg, 25.5 μmol, 63.8% yield, 97.3% purity) as a yellow solid. LCMS [M+23]⁺=509.2.

¹H NMR (400 MHz, DMSO-d₆) δ=9.38 (br s, 1H), 7.68 (s, 1H), 7.64 (s, 1H), 7.03 (br d, J=8.8 Hz, 1H), 6.75 (br d, J=8.0 Hz, 1H), 5.53-5.35 (m, 1H), 4.15-3.90 (m, 2H), 3.86-3.64 (m, 2H), 2.44-2.39 (s, 3H), 2.22-2.09 (m, 2H), 2.01-1.84 (m, 2H), 1.59 (br d, J=6.8 Hz, 3H)

¹⁹F NMR (400 MHz, DMSO-d₆) δ=−99.17, −99.22.

### Examples 13 and 14

### 6-chloro-3-(((R)-1-(2-cyano-3-((((S)-2,2-difluorocyclopropyl)methyl)amino)-7-methylquinoxalin-5-yl)ethyl)amino)picolinic acid (Example 13) and 6-chloro-3-(((R)-1-(2-cyano-3-((((R)-2,2-difluorocyclopropyl)methyl)amino)-7-methylquinoxalin-5-yl)ethyl)amino)picolinic acid (Example 14)

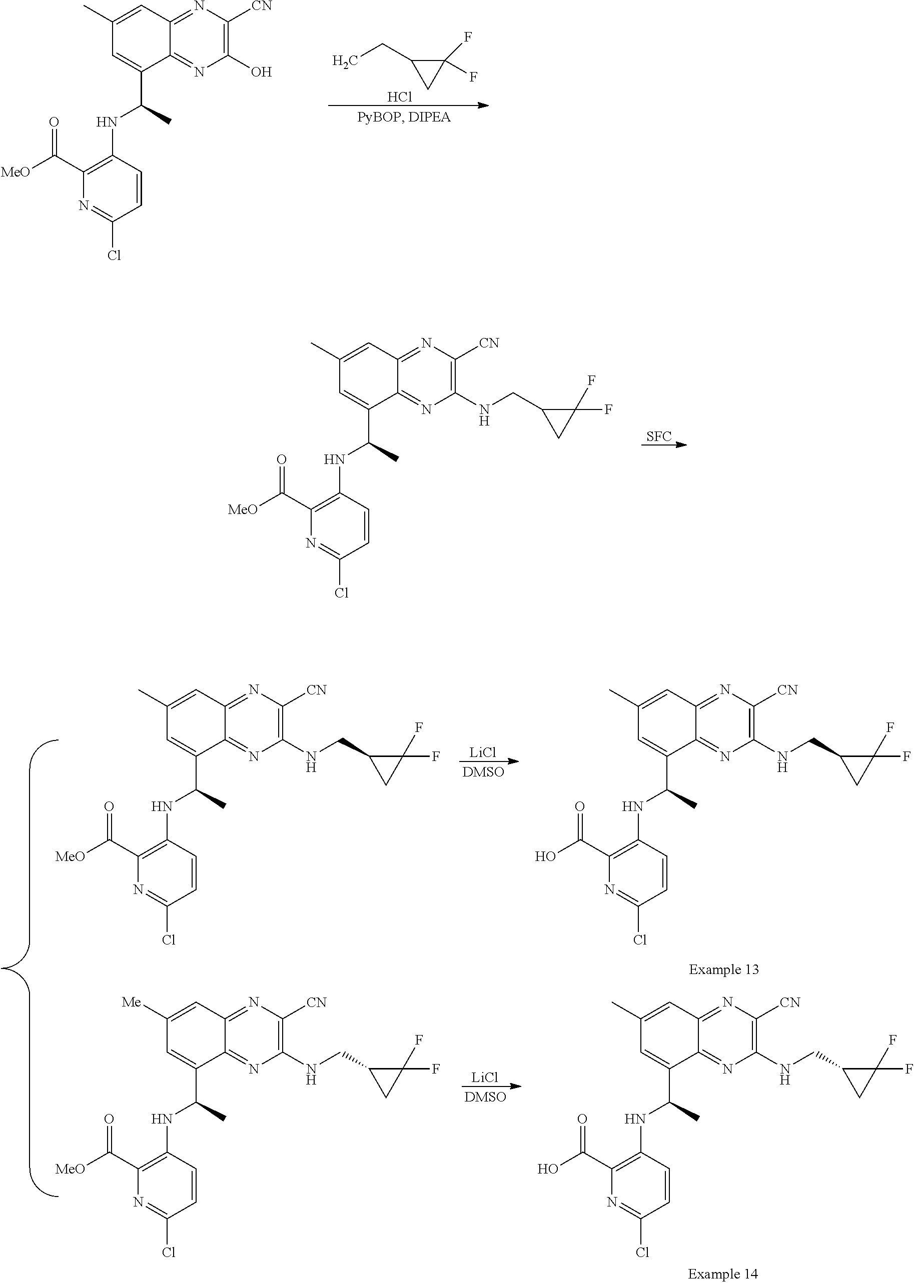

Step A: A solution of methyl (R)-6-chloro-3-((1-(2-cyano-3-hydroxy-7-methylquinoxalin-5-yl)ethyl)amino)picolinate (200 mg, 503 μmol, 1.00 eq.) in N,N-dimethylformamide (2.00 mL) were added benzotriazole-1-yl-oxy-tris-pyrrolidino-phosphonium hexafluorophosphate (392 mg, 754 μmol, 1.50 eq.) and diisopropylethylamine (325 mg, 2.51 mmol, 438 μL, 5.00 eq.). The reaction was stirred at 15° C. for 20 minutes. To the above mixture was added (2,2-difluorocyclopropyl)methanamine hydrochloride (108 mg, 75 μmol, 1.50 eq.). The reaction was stirred at 15° C. for 1 hour. After completion, the mixture was diluted with water (10.0 mL) and extracted with ethyl acetate (10.0 mL×3). The combined organics were washed with brine (30.0 mL), dried over anhydrous sodium sulfate, filtered and concentrated to give a residue. The residue was purified by column chromatography (SiO₂, petroleum ether/ethyl acetate=1/0 to 5/1) and prep-HPLC (column: Phenomenex Luna C18 150×25 mm×10 um; mobile phase: phase A: water (10 mmol/L NH₄HCO₃), phase B: acetonitrile; gradient: 64%-94% phase B over 10 min) to give methyl 6-chloro-3-(((1R)-1-(2-cyano-3-(((2,2-difluorocyclopropyl)methyl)amino)-7-methylquinoxalin-5-yl)ethyl)amino)picolinate (30.0 mg, 61.6 μmol, 12.3% yield) as a yellow solid. LCMS [M+1]⁺=487.2.

Step B: To a mixture of methyl 6-chloro-3-(((1R)-1-(2-cyano-3-(((2,2-difluorocyclopropyl)methyl)amino)-7-methylquinoxalin-5-yl)ethyl)amino)picolinate (30.0 mg, 61.6 μmol, 1.00 eq.) was purified by SFC (column: DAICEL CHIRALPAK AD (250 mm×50 mm, 10 um); mobile phase: phase A: hexanes, phase B: 0.1% ammonia in isopropanol; phase B %: 12%, isocratic elution mode). The desired fractions were collected and concentrated to give the methyl ester of Example 13 (11.0 mg, 22.4 μmol, 36.3% yield, 99.0% purity) as a yellow solid (first eluting isomer LCMS [M+1]⁺=487.3.) and the methyl ester of Example 14 (11.0 mg, 21.9 μmol, 35.6% yield, 97.1% purity) was isolated as a yellow solid (second eluting isomer, LCMS [M+1]⁺=487.3).

### Saponification of the Methyl Esters of Examples 13 and 14

To a solution of the methyl ester of Example 13 (10.0 mg, 20.5 μmol, 1.00 eq.) in dimethyl sulfoxide (0.50 mL) was added lithium chloride (8.71 mg, 205 μmol, 10.0 eq.). The reaction was stirred at 130° C. for 5 hours. After completion, the mixture was cooled to 20° C. The mixture was filtered and the filtrate was purified by prep-HPLC (column: Phenomenex Luna C18 150×25 mm×10 um; mobile phase: phase A: water (10 mmol/L NH₄HCO₃), phase B: acetonitrile; gradient: 22%-52% phase B over 10 min) to give Example 13 (4.12 mg, 8.60 μmol, 41.9% yield, 98.7% purity) as a yellow solid. LCMS [M−1]⁻=471.2.

¹H NMR (400 MHz, DMSO-d₆) δ=9.25 (br s, 1H), 8.00 (t, J=5.6 Hz, 1H), 7.55 (s, 2H), 7.06 (br d, J=8.4 Hz, 1H), 6.73 (br d, J=7.2 Hz, 1H), 5.49-5.35 (m, 1H), 3.82-3.70 (m, 1H), 3.54-3.44 (m, 1H), 2.37 (s, 3H), 2.27-2.13 (m, 1H), 1.67-1.60 (m, 1H), 1.56 (d, J=6.8 Hz, 3H), 1.48-1.35 (m, 1H).

¹⁹F NMR (400 MHz, DMSO-d₆) δ=−126.46, −126.87, −141.37, −141.79.

To a solution of the methyl ester of Example 14 (10.0 mg, 20.5 μmol, 1.00 eq.) in dimethyl sulfoxide (0.50 mL) was added lithium chloride (8.71 mg, 205 μmol, 10.0 eq.). The reaction was stirred at 130° C. for 5 hours. After completion, the mixture was cooled to 20° C. The mixture was filtered and the filtrate was purified by prep-HPLC (column: Phenomenex Luna C18 150×25 mm×10 um; mobile phase: phase A: water (10 mmol/L NH₄HCO₃), phase B: acetonitrile; gradient: 24%-54% phase B over 10 min) to give Example 14 (5.15 mg, 10.8 μmol, 52.5% yield, 99.0% purity) as a yellow solid. LCMS [M−1]⁻=471.2.

¹H NMR (400 MHz, DMSO-d₆) δ=9.25 (br s, 1H), 8.00 (t, J=5.6 Hz, 1H), 7.56 (s, 2H), 7.05 (br d, J=8.8 Hz, 1H), 6.71 (br d, J=8.4 Hz, 1H), 5.49-5.37 (m, 1H), 3.71 (m, 1H), 3.54-3.47 (m, 1H), 2.37 (s, 3H), 2.28-2.14 (m, 1H), 1.64-1.58 (m, 1H), 1.56 (d, J=6.8 Hz, 3H), 1.46-1.33 (m, 1H).

¹⁹F NMR (400 MHz, DMSO-d₆) δ=−126.40, −126.81, −141.44, −141.52.

### Example 15

### (R)-6-chloro-3-((1-(2-cyano-7-methyl-3-(((1-(trifluoromethyl)cyclopropyl)methyl)amino)quinoxalin-5-yl)ethyl)amino)picolinic acid

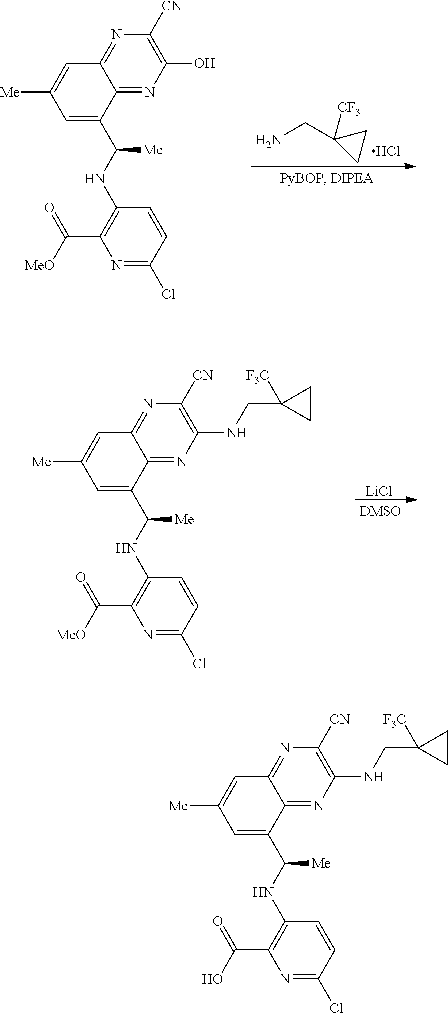 [SMILES: COC(=O)c1nc(Cl)ccc1N[C@H](C)c1cc(C)cc2nc(C#N)c(O)nc12 ; NCC1(C(F)(F)F)CC1 ; Cl ; COC(=O)c1nc(Cl)ccc1N[C@H](C)c1cc(C)cc2nc(C#N)c(NCC3(C(F)(F)F)CC3)nc12 ; "LiCl" ; Cc1cc([C@@H](C)Nc2ccc(Cl)nc2C(=O)O)c2nc(NCC3(C(F)(F)F)CC3)c(C#N)nc2c1]

Step A: To a solution of methyl (R)-6-chloro-3-((1-(2-cyano-3-hydroxy-7-methylquinoxalin-5-yl)ethyl)amino)picolinate (100 mg, 251 μmol, 1.00 eq.), (1-(trifluoromethyl)cyclopropyl)methanamine hydrochloride (53.3 mg, 251 μmol, 1.00 eq., HCl) in N,N-dimethylformamide (1.00 mL) was added benzotriazole-1-yl-oxy-tris-pyrrolidino-phosphonium hexafluorophosphate (157 mg, 302 μmol, 1.20 eq.) and diisopropylethylamine (162 mg, 1.26 mmol, 219 μL, 5.00 eq.). The mixture was stirred at 25° C. for 12 hours. After completion of the reaction, the mixture was cooled to 15° C. and added to water (2.00 mL) slowly, during this period, yellow precipitate was formed, the suspension was filtered, the cake was collected and dried under reduced pressure to give methyl (R)-6-chloro-3-((1-(2-cyano-7-methyl-3-(((1-(trifluoromethyl)cyclopropyl)methyl)amino)quinoxalin-5-yl)ethyl)amino)picolinate (45.0 mg, 71.1 μmol, 28.3% yield, 82% purity) as a yellow solid. LCMS [M+1]⁺=519.2.

Step B: To a solution of methyl (R)-6-chloro-3-((1-(2-cyano-7-methyl-3-(((1-(trifluoromethyl)cyclopropyl)methyl)amino)quinoxalin-5-yl)ethyl)amino)picolinate (45.0 mg, 71.1 μmol, 1.00 eq.) in dimethyl sulfoxide (1.00 mL) was added lithium chloride (30.1 mg, 711 μmol, 14.6 μL, 10.0 eq.). The mixture was stirred at 120° C. for 6 hours. After completion of the reaction, the mixture was cooled to 25° C., and then purified by prep-HPLC (column: Phenomenex luna C18 150×25 mm×10 um; mobile phase: phase A: 0.225% formic acid in water, phase B: acetonitrile; gradient: 62%-92% phase B) to give (R)-6-chloro-3-((1-(2-cyano-7-methyl-3-(((1-(trifluoromethyl)cyclopropyl)methyl)amino)quinoxalin-5-yl)ethyl)amino)picolinic acid (4.26 mg, 8.40 μmol, 11.8% yield, 99.6% purity) as a yellow solid. LCMS [M+1]⁺=505.2.

¹H NMR (400 MHz, CD₃OD) δ=7.57 (s, 2H), 7.17 (d, J=8.8 Hz, 1H), 6.92 (d, J=9.2 Hz, 1H), 5.65-5.54 (m, 1H), 4.14-3.91 (m, 2H), 2.42 (s, 3H), 1.68 (d, J=6.4 Hz, 3H), 1.04-0.93 (m, 4H).

### Example 16

### (R)-6-chloro-3-((1-(2-cyano-3-(1,1-difluoro-5-azaspiro[2.3]hexan-5-yl)-7-methylquinoxalin-5-yl)ethyl)amino)picolinic acid

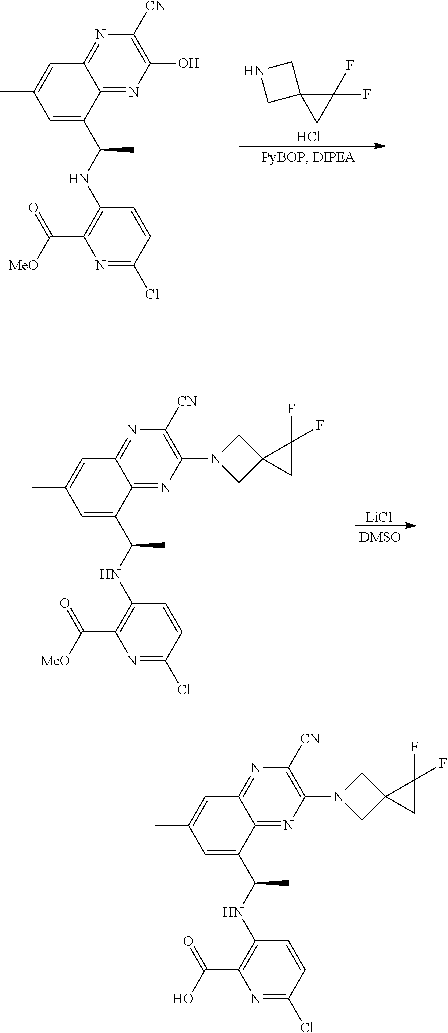 [SMILES: COC(=O)c1nc(Cl)ccc1N[C@H](C)c1cc(C)cc2nc(C#N)c(O)nc12 ; FC1(F)CC12CNC2 ; Cl ; COC(=O)c1nc(Cl)ccc1N[C@H](C)c1cc(C)cc2nc(C#N)c(N3CC4(C3)CC4(F)F)nc12 ; "LiCl" ; Cc1cc([C@@H](C)Nc2ccc(Cl)nc2C(=O)O)c2nc(N3CC4(C3)CC4(F)F)c(C#N)nc2c1]

Step A: To a solution of methyl (R)-6-chloro-3-((1-(2-cyano-3-hydroxy-7-methylquinoxalin-5-yl)ethyl)amino)picolinate (50.0 mg, 126 μmol, 1.00 eq.), benzotriazole-1-yl-oxy-tris-pyrrolidino-phosphonium hexafluorophosphate (98.1 mg, 188 μmol, 1.50 eq.) and diisopropylethylamine (81.2 mg, 628 μmol, 109 μL, 5.00 eq.) in N,N-dimethylformamide (1.00 mL) was stirred at 20° C. for 20 minutes. To the above mixture was added 1 1,1-difluoro-5-azaspiro[2.3]hexane hydrochloride (29.3 mg, 188 μmol, 1.50 eq.) at 20° C. The reaction was stirred at 20° C. for 1 hour. After completion, the mixture was diluted with water (3.00 mL) and extracted with ethyl acetate (3×3.00 mL). The combined organic layers were dried over anhydrous sodium sulfate and concentrated to give a residue. The residue was purified by prep-TLC (SiO₂, petroleum ether:ethyl acetate=1:3) to give methyl (R)-6-chloro-3-((1-(2-cyano-3-(1,1-difluoro-5-azaspiro[2.3]hexan-5-yl)-7-methylquinoxalin-5-yl)ethyl)amino)picolinate (55.0 mg, 75.0 μmol, 59.6% yield, 68.0% purity) as a yellow solid. LCMS [M+1]⁺=499.2.

Step B: To a solution of methyl (R)-6-chloro-3-((1-(2-cyano-3-(1,1-difluoro-5-azaspiro[2.3]hexan-5-yl)-7-methylquinoxalin-5-yl)ethyl)amino)picolinate (50.0 mg, 90.2 μmol, 1.00 eq.) in dimethyl sulfoxide (1.00 mL) was added lithium chloride (38.2 mg, 902 μmol, 10.0 eq.). The reaction was stirred at 130° C. for 2 hours. After completion, the mixture was cooled to 20° C. The mixture was filtered and the filtrate was purified by prep-HPLC (column: YMC-Actus Triart C18 150*30 mm*7 um; mobile phase: phase A: 0.225% formic acid in water, phase B: acetonitrile; gradient: 63%-93% phase B) to give (R)-6-chloro-3-((1-(2-cyano-3-(1,1-difluoro-5-azaspiro[2.3]hexan-5-yl)-7-methylquinoxalin-5-yl)ethyl)amino)picolinic acid (14.4 mg, 29.7 μmol, 32.9% yield, 99.9% purity) as a yellow solid. LCMS [M−1]⁻=483.0.

¹H NMR (400 MHz, DMSO-d₆) δ=12.96 (br s, 1H), 8.57 (br d, J=7.2 Hz, 1H), 7.66 (s, 1H), 7.64 (s, 1H), 7.30 (d, J=8.8 Hz, 1H), 7.09 (d, J=8.8 Hz, 1H), 5.41 (quin, J=12.8 Hz, 1H), 4.66-4.51 (m, 4H), 2.41 (s, 3H), 1.88 (br t, J=8.8 Hz, 2H), 1.66 (d, J=6.8 Hz, 3H).

¹⁹F NMR (400 MHz, DMSO-d₆) δ=−137.62.

### Example 17

### (R)-3-((1-(2-cyano-3-(4,4-difluoropiperidin-1-yl)-7-methylquinoxalin-5-yl)ethyl)amino)-6-methylpicolinic acid

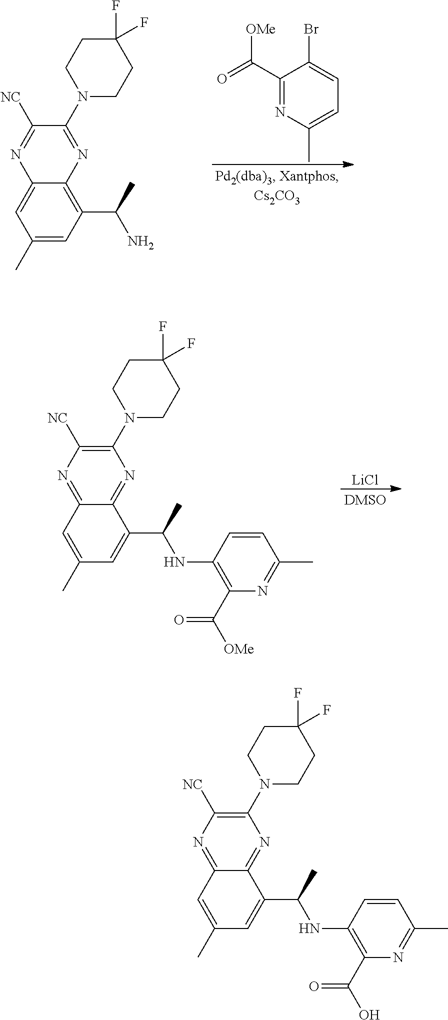 [SMILES: Cc1cc([C@@H](C)N)c2nc(N3CCC(F)(F)CC3)c(C#N)nc2c1 ; COC(=O)c1nc(C)ccc1Br ; COC(=O)c1nc(C)ccc1N[C@H](C)c1cc(C)cc2nc(C#N)c(N3CCC(F)(F)CC3)nc12 ; "LiCl" ; Cc1cc([C@@H](C)Nc2ccc(C)nc2C(=O)O)c2nc(N3CCC(F)(F)CC3)c(C#N)nc2c1]

Step A: To a solution of (R)-5-(1-aminoethyl)-3-(4,4-difluoropiperidin-1-yl)-7-methylquinoxaline-2-carbonitrile (100 mg, 272 μmol, 1.00 eq, hydrochloride salt), methyl 3-bromo-6-methylpicolinate (125. mg, 544 μmol, 2.00 eq.), Xantphos (31.5 mg, 54.4 μmol, 0.20 eq.), cesium carbonate (354 mg, 1.09 mmol, 4.00 eq.) in dioxane (2.00 mL) was added tris(dibenzylideneacetone)dipalladium(0) (24.9 mg, 27.2 μmol, 0.10 eq.). The mixture was stirred at 100° C. for 12 hours under a nitrogen atmosphere. After completion of the reaction, the mixture was cooled to 25° C., and then directly concentrated to give a residue. The residue was purified by prep-TLC (SiO₂, petroleum ether/ethyl acetate=3:1) to give methyl (R)-3-((1-(2-cyano-3-(4,4-difluoropiperidin-1-yl)-7-methylquinoxalin-5-yl)ethyl)amino)-6-methylpicolinate (100 mg, 208 μmol, 76.6% yield) as a yellow solid. LCMS [M+1]⁺=481.2.

Step B: A mixture of methyl (R)-3-((1-(2-cyano-3-(4,4-difluoropiperidin-1-yl)-7-methylquinoxalin-5-yl)ethyl)amino)-6-methylpicolinate in dimethyl sulfoxide (0.5 mL) was added lithium chloride (79.4 mg, 1.87 mmol, 38.4 μL, 10.0 eq.). The reaction was stirred at 120° C. for 8 hours. After completion of the reaction, the mixture was cooled to 25° C., and then directly concentrated to give a residue. The reaction mixture was diluted with water (10.0 mL) and extracted with ethyl acetate (20.0 mL×2). The combined organic layers were washed with saturate sodium bicarbonate solution (10.0 mL×2), dried over anhydrous sodium sulfate and concentrated under reduced pressure to give a residue. The residue was purified by column chromatography (SiO₂, petroleum ether/ethyl acetate=1/1 to 0/1) to give (R)-3-((1-(2-cyano-3-(4,4-difluoropiperidin-1-yl)-7-methylquinoxalin-5-yl)ethyl)amino)-6-methylpicolinic acid (25.6 mg, 53.7 μmol, 28.7% yield, 97.9% purity) as a yellow solid. LCMS [M+1]⁺=467.1.

¹H NMR (400 MHz, CDCl₃) δ=8.09 (br d, J=5.2 Hz, 1H), 7.58 (s, 1H), 7.53 (s, 1H), 6.92 (d, J=8.8 Hz, 1H), 6.61 (d, J=8.8 Hz, 1H), 5.43 (quin, J=6.6 Hz, 1H), 3.88-3.75 (m, 4H), 2.39 (s, 3H), 2.31 (s, 3H), 2.24-2.14 (m, 4H), 1.61 (d, J=6.8 Hz, 3H).

### Examples 18-105

The compounds in Table 1 are prepared essentially according to the procedures set forth in the above schemes and examples, or as set forth in schemes and examples immediately below Table 1.

TABLE 1

| Example | | |
|---|---|---|
| No. | Structure | Spectral Data |
| 18 | 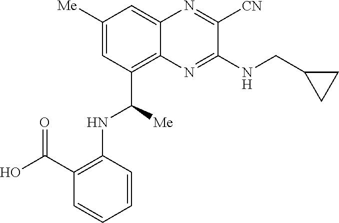 [SMILES: Cc1cc([C@@H](C)Nc2ccccc2C(=O)O)c2nc(NCC3CC3)c(C#N)nc2c1] | ¹H NMR (400 MHz, CDCl₃) δ = 8.22 (br s, 1H), 7.99 (dd, J = 1.6, 8.0 Hz, 1H), 7.56 (s, 2H), 7.18 (ddd, J = 1.6, 7.2, 8.4 Hz, 1H), 6.58 (t, J = 7.6 Hz, 1H), 6.38 (d, J = 8.4 Hz, 1H), 5.69- 5.46 (m, 2H), 3.54-3.44 (m, 2H), 2.43 (s, 3H), 1.67 (d, J = 6.8 Hz, 3H), 1.26-1.18 (m, 1H), 0.71-0.59 (m, 2H), 0.38 (q, J = 4.8 Hz, 2H). LCMS [M + 1]⁺ = 402.2. |
| | (R)-2-((1-(2-cyano-3- | |
| | ((cyclopropylmethyl)amino)-7-methylquinoxalin- | |
| | 5-yl)ethyl)amino)benzoic acid | |
| 19 | 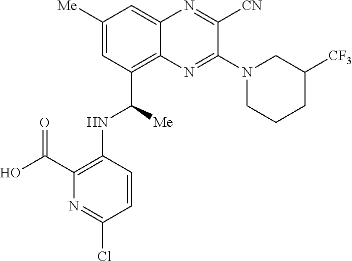 [SMILES: Cc1cc([C@@H](C)Nc2ccc(Cl)nc2C(=O)O)c2nc(N3CCCC(C(F)(F)F)C3)c(C#N)nc2c1] | ¹H NMR (400 MHz, DMSO-d₆) δ = 12.95 (br s, 1H), 8.56- 8.44 (m, 1H), 7.72-7.64 (m, 2H), 7.32-7.25 (m, 1H), 7.10- 6.95 (m, 1H), 5.51-5.39 (m, 1H), 4.50 (br t, J = 9.2 Hz, 1H), 4.42-4.31 (m, 1H), 3.25-3.14 (m, 2H), 2.91-2.74 (m, 1H), 2.43 (d, J = 2.0 Hz, 3H), 2.07- 2.00 (m, 1H), 1.93-1.84 (m, 1H), 1.76-1.62 (m, 5H). ¹⁹F NMR (400 MHz, DMSO-d₆) δ = −71.01. LCMS [M + 23]⁺ = 541.2. |
| | 6-chloro-3-(((1R)-1-(2-cyano-7-methyl-3-(3- | |
| | (trifluoromethyl)piperidin-1-yl)quinoxalin-5- | |
| | yl)ethyl)amino)picolinic acid | |
| 20 | 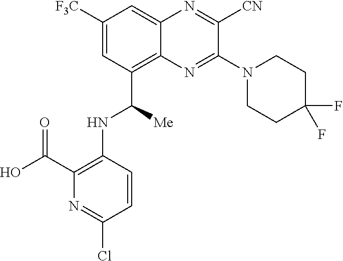 [SMILES: C[C@@H](Nc1ccc(Cl)nc1C(=O)O)c1cc(C(F)(F)F)cc2nc(C#N)c(N3CCC(F)(F)CC3)nc12] | ¹H NMR (400 MHz, DMSO-d₆) δ = 8.57-8.46 (m, 1H), 8.30 (d, J = 1.2 Hz, 1H), 8.02 (d, J = 2.0 Hz, 1H), 7.30 (d, J = 8.8 Hz, 1H), 7.09 (d, J = 8.8 Hz, 1H), 5.58-5.46 (m, 1H), 4.11- 3.93 (m, 4H), 2.35-2.09 (m, 5H), 1.69 (d, J = 6.8 Hz, 3H). ¹⁹F NMR (400 MHz, DMSO-d₆) δ = −60.93, −94.78. LCMS [M + 1]⁺ = 541.1. |
| | (R)-6-chloro-3-((1-(2-cyano-3-(4,4- | |
| | difluoropiperidin-1-yl)-7- | |
| | (trifluoromethyl)quinoxalin-5- | |
| | yl)ethyl)amino)picolinic acid | |
| 21 | 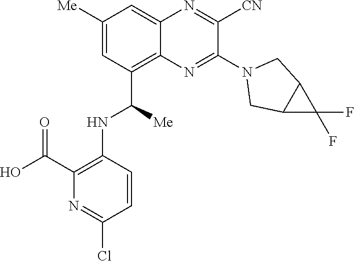 [SMILES: Cc1cc([C@@H](C)Nc2ccc(Cl)nc2C(=O)O)c2nc(N3CC4C(C3)C4(F)F)c(C#N)nc2c1] | ¹H NMR (400 MHz, DMSO-d₆) δ = 9.02-8.78 (m, 1H), 7.59 (s, 2H), 7.17 (d, J = 8.8 Hz, 1H), 6.87 (d, J = 8.8 Hz, 1H), 5.39 (br d, J = 4.0 Hz, 1H), 4.38-4.12 (m, 4H), 2.83 (br d, J = 10.4 Hz, 2H), 2.39 (s, 3H), 1.62 (d, J = 6.8 Hz, 3H). ¹⁹F NMR (400 MHz, DMSO-d₆) δ = −129.01, −129.43, −154.05, −154.48 LCMS [M + 23]⁺ = 507.2. |
| | 6-chloro-3-(((1R)-1-(2-cyano-3-(6,6-difluoro-3- | |
| | azabicyclo[3.1.0]hexan-3-yl)-7-methylquinoxalin- | |
| | 5-yl)ethyl)amino)picolinic acid | |
| 22 | 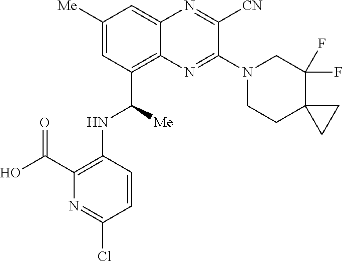 [SMILES: Cc1cc([C@@H](C)Nc2ccc(Cl)nc2C(=O)O)c2nc(N3CCC4(CC4)C(F)(F)C3)c(C#N)nc2c1] | ¹H NMR (400 MHz, DMSO-d₆) δ = 9.05-8.81 (br s, 1H), 7.70 (s, 1H), 7.67 (d, J = 1.6 Hz, 1H), 7.15 (d, J = 8.8 Hz, 1H), 6.89 (br d, J = 8.8 Hz, 1H), 5.47-5.44 (m, 1H), 4.11-4.01 (m, 2H), 3.94-3.79 (m, 2H), 2.43 (s, 3H), 1.94-1.79 (m, 2H), 1.63 (d, J = 6.4 Hz, 3H), 0.94-0.86 (m, 2H), 0.63 (br s, 2H). ¹⁹F NMR (400 MHz, DMSO-d₆) δ = −110.16, −110.40. LCMS [M + 23]⁺ = 535.2. |
| | (R)-6-chloro-3-((1-(2-cyano-3-(4,4-difluoro-6- | |
| | azaspiro[2.5]octan-6-yl)-7-methylquinoxalin-5- | |
| | yl)ethyl)amino)picolinic acid | |
| 23 | 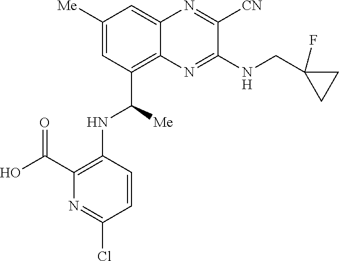 [SMILES: Cc1cc([C@@H](C)Nc2ccc(Cl)nc2C(=O)O)c2nc(NCC3(F)CC3)c(C#N)nc2c1] | ¹H NMR (400 MHz, methanol- d₄) δ = 7.57 (dd, J = 2.0, 6.8 Hz, 2H), 7.16 (d, J = 8.8 Hz, 1H), 6.96 (d, J = 9.2 Hz, 1H), 5.56 (q, J = 6.8 Hz, 1H), 4.19 (dd, J = 14.8, 18.8 Hz, 1H), 3.97 (dd, J = 14.8, 21.6 Hz, 1H), 2.42 (s, 3H), 1.67 (d, J = 6.8 Hz, 3H), 1.14-0.97 (m, 2H), 0.93-0.78 (m, 2H). ¹⁹F NMR (400 MHz, methanol-d₄) δ = −186.97. LCMS [M + H]⁺ = 455.1. |
| | (R)-6-chloro-3-((1-(2-cyano-3-(((1- | |
| | fluorocyclopropyl)methyl)amino)-7- | |
| | methylquinoxalin-5-yl)ethyl)amino)picolinic acid | |
| 24 | 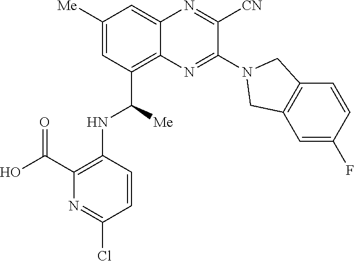 [SMILES: Cc1cc([C@@H](C)Nc2ccc(Cl)nc2C(=O)O)c2nc(N3Cc4ccc(F)cc4C3)c(C#N)nc2c1] | ¹H NMR (400 MHz, DMSO-d₆) δ = 13.15-12.80 (m, 1H), 8.55 (br d, J = 7.6 Hz, 1H), 7.63 (d, J = 7.6 Hz, 2H), 7.53 (dd, J = 5.2, 8.4 Hz, 1H), 7.43-7.29 (m, 2H), 7.25-7.15 (m, 1H), 7.11 (d, J = 9.2 Hz, 1H), 5.51 (quin, J = 6.8 Hz, 1H), 5.39- 5.09 (m, 4H), 2.41 (s, 3H), 1.71 (d, J = 6.8 Hz, 3H). ¹⁹F NMR (400 MHz, DMSO-d₆) δ = −115.29. LCMS [M + 1]⁺ = 503.2. |
| | (R)-6-chloro-3-((1-(2-cyano-3-(5-fluoroisoindolin- | |
| | 2-yl)-7-methylquinoxalin-5- | |
| | yl)ethyl)amino)picolinic acid | |
| 25 | 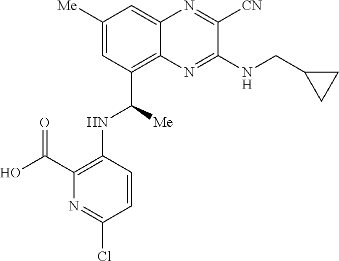 [SMILES: Cc1cc([C@@H](C)Nc2ccc(Cl)nc2C(=O)O)c2nc(NCC3CC3)c(C#N)nc2c1] | ¹H NMR (400 MHz, DMSO-d₆) δ = 8.66-8.46 (br s, 1H), 7.82 (t, J = 5.6 Hz, 1H), 7.55 (s, 2H), 7.27 (d, J = 9.2 Hz, 1H), 6.95 (d, J = 9.2 Hz, 1H), 5.51- 5.40 (m, 1H), 3.47-3.39 (m, 2H), 2.37 (s, 3H), 1.61 (d, J = 6.8 Hz, 3H), 1.27-1.20 (m, 1H), 0.49-0.41 (m, 2H), 0.34- 0.26 (m, 2H). LCMS [M + 23]⁺ = 459.3. |
| | (R)-6-chloro-3-((1-(2-cyano-3- | |
| | ((cyclopropylmethyl)amino)-7-methylquinoxalin- | |
| | 5-yl)ethyl)amino)picolinic acid | |
| 26 | 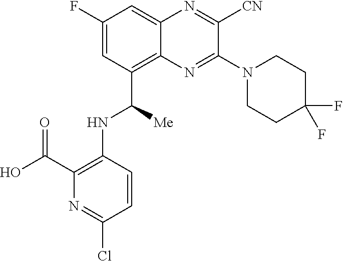 [SMILES: C[C@@H](Nc1ccc(Cl)nc1C(=O)O)c1cc(F)cc2nc(C#N)c(N3CCC(F)(F)CC3)nc12] | ¹H NMR (400 MHz, DMSO-d₆) δ = 13.05 (br s, 1H), 8.45 (br d, J = 6.8 Hz, 1H), 7.77 (d, J = 9.2 Hz, 2H), 7.29 (d, J = 8.8 Hz, 1H), 7.02 (d, J = 9.2 Hz, 1H), 5.60-5.45 (m, 1H), 3.90 (s, 4H), 2.29-2.15 (m, 4H), 1.67 (d, J = 6.8 Hz, 3H). ¹⁹F NMR (400 MHz, DMSO-d₆) δ = −94.71, −111.49. LCMS [M + 1]⁺ = 491.2. |
| | (R)-6-chloro-3-((1-(2-cyano-3-(4,4- | |
| | difluoropiperidin-1-yl)-7-fluoroquinoxalin-5- | |
| | yl)ethyl)amino)picolinic acid | |
| 27 | 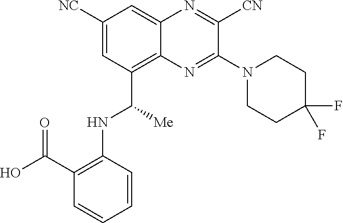 [SMILES: C[C@H](Nc1ccccc1C(=O)O)c1cc(C#N)cc2nc(C#N)c(N3CCC(F)(F)CC3)nc12] | ¹H NMR (400 MHz, methanol- d₄) δ = 8.28 (d, J = 1.6 Hz, 1H), 7.96-7.86 (m, 2H), 7.14 (t, J = 7.2 Hz, 1H), 6.56 (t, J = 7.6 Hz, 1H), 6.39 (d, J = 8.4 Hz, 1H), 5.57 (q, J = 6.8 Hz, 1H), 4.17-4.03 (m, 4H), 2.35- 2.19 (m, 4H), 1.71 (d, J = 6.8 Hz, 3H). ¹⁹F NMR (400 MHz, CD3OD) δ = −98.62. LCMS [M + H]⁺ = 463.1. |
| | (S)-2-((1-(2,7-dicyano-3-(4,4-difluoropiperidin-1- | |
| | yl)quinoxalin-5-yl)ethyl)amino)benzoic acid | |
| 28 | 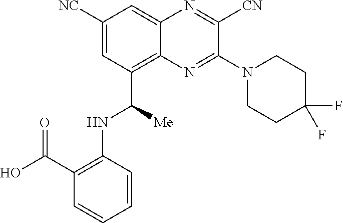 [SMILES: C[C@@H](Nc1ccccc1C(=O)O)c1cc(C#N)cc2nc(C#N)c(N3CCC(F)(F)CC3)nc12] | ¹H NMR (400 MHz, methanol- d₄) δ = 8.28 (d, J = 1.6 Hz, 1H), 7.96-7.86 (m, 2H), 7.14 (t, J = 7.2 Hz, 1H), 6.56 (t, J = 7.6 Hz, 1H), 6.39 (d, J = 8.4 Hz, 1H), 5.57 (q, J = 6.8 Hz, 1H), 4.17-4.03 (m, 4H), 2.35- 2.19 (m, 4H), 1.71 (d, J = 6.8 Hz, 3H). ¹⁹F NMR (400 MHz, CD3OD) δ = −98.62. LCMS [M + H]⁺ = 463.1. |
| | (R)-2-((1-(2,7-dicyano-3-(4,4-difluoropiperidin-1- | |
| | yl)quinoxalin-5-yl)ethyl)amino)benzoic acid | |
| 29 | 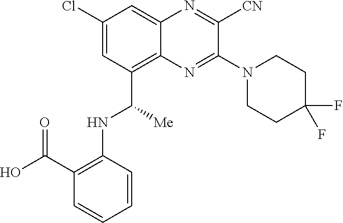 [SMILES: C[C@H](Nc1ccccc1C(=O)O)c1cc(Cl)cc2nc(C#N)c(N3CCC(F)(F)CC3)nc12] | ¹H NMR (400 MHz, methanol- d₄) δ = 7.78 (dd, J = 1.2, 7.6 Hz, 1H), 7.70 (d, J = 2.4 Hz, 1H), 7.59 (d, J = 2.4 Hz, 1H), 7.04-6.94 (m, 1H), 6.40 (t, J = 7.6 Hz, 1H), 6.24 (d, J = 8.4 Hz, 1H), 5.45 (q, J = 6.8 Hz, 1H), 3.92-3.78 (m, 4H), 2.21- 2.04 (m, 4H), 1.57 (d, J = 6.8 Hz, 3H). ¹⁹F NMR (400 MHz, methanol-d₄) δ = −98.51. LCMS [M + H]⁺ = 472.3. |
| | (S)-2-((1-(7-chloro-2-cyano-3-(4,4- | |
| | difluoropiperidin-1-yl)quinoxalin-5- | |
| | yl)ethyl)amino)benzoic acid | |
| 30 | 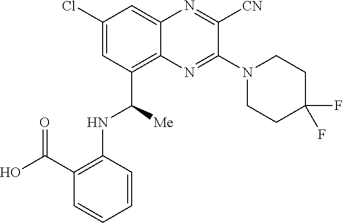 [SMILES: C[C@@H](Nc1ccccc1C(=O)O)c1cc(Cl)cc2nc(C#N)c(N3CCC(F)(F)CC3)nc12] | ¹H NMR (400 MHz, methanol- d₄) δ = 7.78 (dd, J = 1.2, 7.6 Hz, 1H), 7.70 (d, J = 2.4 Hz, 1H), 7.59 (d, J = 2.4 Hz, 1H), 7.04-6.94 (m, 1H), 6.40 (t, J = 7.6 Hz, 1H), 6.24 (d, J = 8.4 Hz, 1H), 5.45 (q, J = 6.8 Hz, 1H), 3.92-3.78 (m, 4H), 2.21- 2.04 (m, 4H), 1.57 (d, J = 6.8 Hz, 3H). ¹⁹F NMR (400 MHz, CD₃OD) δ = −98.51. LCMS [M + H]⁺ = 472.3. |
| | (R)-2-((1-(7-chloro-2-cyano-3-(4,4- | |
| | difluoropiperidin-1-yl)quinoxalin-5- | |
| | yl)ethyl)amino)benzoic acid | |
| 31 | 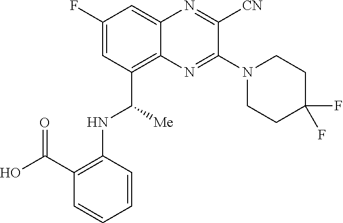 [SMILES: C[C@H](Nc1ccccc1C(=O)O)c1cc(F)cc2nc(C#N)c(N3CCC(F)(F)CC3)nc12] | ¹H NMR (400 MHz, methanol- d4) δ 7.90 (br d, J = 8.4 Hz, 1H), 7.59 (dd, J = 9.6, 2.4 Hz, 1H), 7.47-7.55 (m, 1H), 7.04- 7.16 (m, 1H), 6.53 (br t, J = 7.2 Hz, 1H), 6.34 (d, J = 8.4 Hz, 1H), 5.60 (q, J = 6.4 Hz, 1H), 3.89-3.97 (m, 4H), 2.14-2.34 (m, 4H), 1.68 (d, J = 6.8 Hz, 3H). ¹⁹F NMR (400 MHz, DMSO-d₆) δ = −98.53, −112.81. LCMS [M + 1]⁺ = 456.1. |
| | (S)-2-((1-(2-cyano-3-(4,4-difluoropiperidin-1-yl)- | |
| | 7-fluoroquinoxalin-5-yl)ethyl)amino)benzoic acid | |
| 32 | 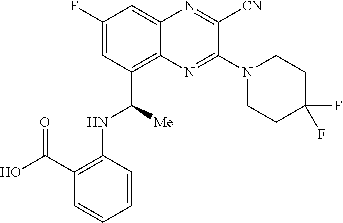 [SMILES: C[C@@H](Nc1ccccc1C(=O)O)c1cc(F)cc2nc(C#N)c(N3CCC(F)(F)CC3)nc12] | ¹H NMR (400 MHz, methanol- d₄) δ 7.90 (br d, J = 8.0 Hz, 1H), 7.59 (br d, J = 9.6 Hz, 1H), 7.46-7.55 (m, 1H), 7.02- 7.15 (m, 1H), 6.53 (br t, J = 7.2 Hz, 1H), 6.33 (d, J = 8.4 Hz, 1H), 5.60 (q, J = 6.4 Hz, 1H), 3.89-3.97 (m, 4H), 2.13-2.36 (m, 4H), 1.68 (br d, J = 6.8 Hz, 3H). ¹⁹F NMR (400 MHz, DMSO-d₆) δ = −98.53, −112.81. LCMS [M + 1]⁺ = 456.1. |
| | (R)-2-((1-(2-cyano-3-(4,4-difluoropiperidin-1-yl)- | |
| | 7-fluoroquinoxalin-5-yl)ethyl)amino)benzoic acid | |
| 33 | 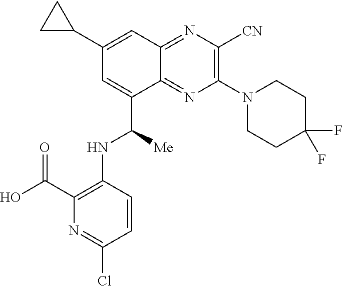 [SMILES: C[C@@H](Nc1ccc(Cl)nc1C(=O)O)c1cc(C2CC2)cc2nc(C#N)c(N3CCC(F)(F)CC3)nc12] | ¹H NMR (400 MHz, CDCl₃) δ = 10.69 (br s, 1H), 8.37 (br d, J = 6.4 Hz, 1H), 7.53 (d, J = 2.0 Hz, 1H), 7.49 (d, J = 2.0 Hz, 1H), 7.13 (d, J = 8.8 Hz, 1H), 6.76 (d, J = 8.8 Hz, 1H), 5.48 (quin, J = 6.8 Hz, 1H), 4.01-3.80 (m, 4H), 2.36-2.18 (m, 4H), 2.05- 1.93 (m, 1H), 1.72 (d, J = 6.8 Hz, 3H), 1.16-1.03 (m, 2H), 0.85-0.76 (m, 1H), 0.76-0.66 (m, 1H). LCMS [M + 1]⁺: 513.1. |
| | (R)-6-chloro-3-((1-(2-cyano-7-cyclopropyl-3-(4,4- | |
| | difluoropiperidin-1-yl)quinoxalin-5- | |
| | yl)ethyl)amino)picolinic acid | |
| 34 | 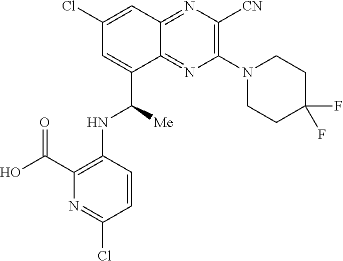 [SMILES: C[C@@H](Nc1ccc(Cl)nc1C(=O)O)c1cc(Cl)cc2nc(C#N)c(N3CCC(F)(F)CC3)nc12] | LCMS [M + 1]⁺: 507.1. |
| | (R)-6-chloro-3-((1-(7-chloro-2-cyano-3-(4,4- | |
| | difluoropiperidin-1-yl)quinoxalin-5- | |
| | yl)ethyl)amino)picolinic acid | |
| 35 | 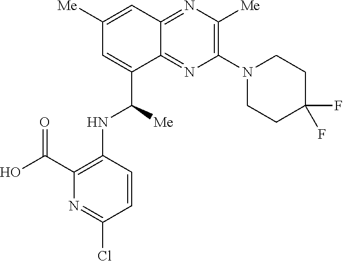 [SMILES: Cc1cc([C@@H](C)Nc2ccc(Cl)nc2C(=O)O)c2nc(N3CCC(F)(F)CC3)c(C)nc2c1] | ¹H NMR (400 MHz, DMSO-d₆) δ = 12.94 (br s, 1H), 8.49 (d, J = 6.8 Hz, 1H), 7.56 (s, 1H), 7.44 (d, J = 1.6 Hz, 1H), 7.30 (d, J = 9.2 Hz, 1H), 7.06 (d, J = 9.2 Hz, 1H), 5.52 (quin, J = 6.8 Hz, 1H), 3.57-3.45 (m, 4H), 2.67 (s, 3H), 2.41 (s, 3H), 2.28-2.13 (m, 4H), 1.66 (d, J = 6.8 Hz, 3H). LCMS [M + 1]⁺ = 476.1. |
| | (R)-6-chloro-3-((1-(3-(4,4-difluoropiperidin-1-yl)- | |
| | 2,7-dimethylquinoxalin-5-yl)ethyl)amino)picolinic | |
| | acid | |
| 36 | 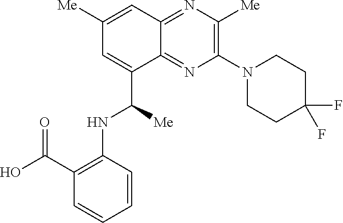 [SMILES: Cc1cc([C@@H](C)Nc2ccccc2C(=O)O)c2nc(N3CCC(F)(F)CC3)c(C)nc2c1] | ¹H NMR (400 MHz, DMSO-d₆) δ = 12.64 (br s, 1H), 8.47 (d, J = 6.0 Hz, 1H), 7.77 (dd, J = 2.8 Hz, 1.6 Hz, 1H), 7.54 (s, 1H), 7.42 (s, 1H), 7.19-7.10 (m, 1H), 6.54-6.40 (m, 2H), 5.56 (m, 1H), 3.52 (m, 4H), 2.68 (s, 3H), 2.40 (s, 3H), 2.28-2.14 (m, 4H), 1.61 (d, J = 6.8 Hz, 3H). LCMS [M + 1]⁺ = 441.2. |
| | (R)-2-((1-(3-(4,4-difluoropiperidin-1-yl)-2,7- | |
| | dimethylquinoxalin-5-yl)ethyl)amino)benzoic | |
| | acid | |
| 37 | 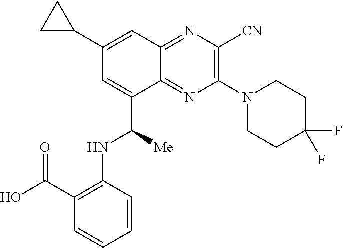 [SMILES: C[C@@H](Nc1ccccc1C(=O)O)c1cc(C2CC2)cc2nc(C#N)c(N3CCC(F)(F)CC3)nc12] | ¹H NMR (400 MHz, CDCl₃) δ = 8.24 (br s, 1H), 8.03 (dd, J = 1.2, 8.0 Hz, 1H), 7.61 (d, J = 2.0 Hz, 1H), 7.50 (d, J = 2.0 Hz, 1H), 7.26-7.18 (m, 1H), 6.62 (t, J = 7.2 Hz, 1H), 6.37 (d, J = 8.4 Hz, 1H), 5.62-5.52 (m, 1H), 4.01-3.83 (m, 4H), 2.36- 2.19 (m, 4H), 2.07-1.96 (m, 1H), 1.72 (d, J = 6.8 Hz, 3H), 1.08 (dd, J = 1.6, 8.4 Hz, 2H), 0.88-0.80 (m, 1H), 0.77-0.69 (m, 1H). LCMS [M + 1]⁺: 478.2. |
| | (R)-2-((1-(2-cyano-7-cyclopropyl-3-(4,4- | |
| | difluoropiperidin-1-yl)quinoxalin-5- | |
| | yl)ethyl)amino)benzoic acid | |
| 38 | 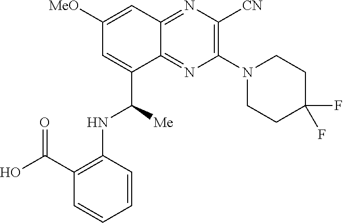 [SMILES: COc1cc([C@@H](C)Nc2ccccc2C(=O)O)c2nc(N3CCC(F)(F)CC3)c(C#N)nc2c1] | |
| | (R)-2-((1-(2-cyano-3-(4,4-difluoropiperidin-1-yl)- | |
| | 7-methoxyquinoxalin-5-yl)ethyl)amino)benzoic | |
| | acid | |
| 39 | 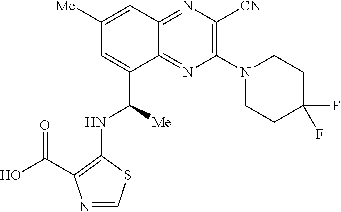 [SMILES: Cc1cc([C@@H](C)Nc2scnc2C(=O)O)c2nc(N3CCC(F)(F)CC3)c(C#N)nc2c1] | ¹H NMR (400 MHz, CDCl₃) δ = 8.03 (br d, J = 7.6 Hz, 1H), 7.73 (s, 1H), 7.71 (s, 1H), 7.59 (d, J = 1.6 Hz, 1H), 5.16 (quin, J = 6.8 Hz, 1H), 3.97-3.80 (m, 4H), 2.52 (s, 3H), 2.32-2.22 (m, 4H), 1.79 (d, J = 6.8 Hz, 3H). LCMS [M + 1]⁺ = 459.1. |
| | (R)-5-((1-(2-cyano-3-(4,4-difluoropiperidin-1-yl)- | |
| | 7-methylquinoxalin-5-yl)ethyl)amino)thiazole-4- | |
| | carboxylic acid | |
| 40 | 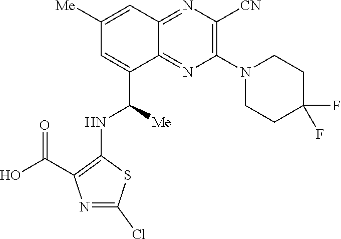 [SMILES: Cc1cc([C@@H](C)Nc2sc(Cl)nc2C(=O)O)c2nc(N3CCC(F)(F)CC3)c(C#N)nc2c1] | ¹H NMR (400 MHz, CDCl₃) δ = 8.10 (br d, J = 7.6 Hz, 1H), 7.73 (d, J = 0.8 Hz, 1H), 7.57 (d, J = 2.0 Hz, 1H), 5.08 (quin, J = 6.8 Hz, 1H), 3.96-3.82 (m, 4H), 2.54 (s, 3H), 2.33-2.22 (m, 4H), 1.78 (d, J = 6.8 Hz, 3H). LCMS [M + Na]⁺ = 515.1. |
| | (R)-2-chloro-5-((1-(2-cyano-3-(4,4- | |
| | difluoropiperidin-1-yl)-7-methylquinoxalin-5- | |
| | yl)ethyl)amino)thiazole-4-carboxylic acid | |
| 41 | 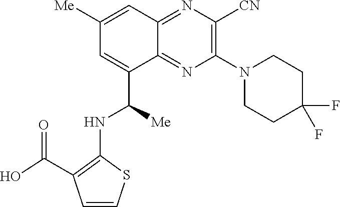 [SMILES: Cc1cc([C@@H](C)Nc2sccc2C(=O)O)c2nc(N3CCC(F)(F)CC3)c(C#N)nc2c1] | |
| | (R)-2-((1-(2-cyano-3-(4,4-difluoropiperidin-1-yl)- | |
| | 7-methylquinoxalin-5-yl)ethyl)amino)thiophene- | |
| | 3-carboxylic acid | |
| 42 | 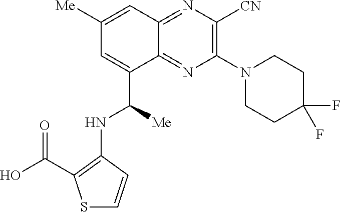 [SMILES: Cc1cc([C@@H](C)Nc2ccsc2C(=O)O)c2nc(N3CCC(F)(F)CC3)c(C#N)nc2c1] | |
| | (R)-3-((1-(2-cyano-3-(4,4-difluoropiperidin-1-yl)- | |
| | 7-methylquinoxalin-5-yl)ethyl)amino)thiophene- | |
| | 2-carboxylic acid | |
| 43 | 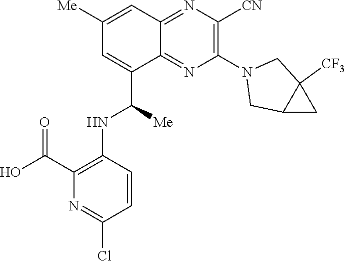 [SMILES: Cc1cc([C@@H](C)Nc2ccc(Cl)nc2C(=O)O)c2nc(N3CC4CC4(C(F)(F)F)C3)c(C#N)nc2c1] | |
| | 6-chloro-3-(((1R)-1-(2-cyano-7-methyl-3-(1- | |
| | (trifluoromethyl)-3-azabicyclo[3.1.0]hexan-3- | |
| | yl)quinoxalin-5-yl)ethyl)amino)picolinic acid | |
| 44 | 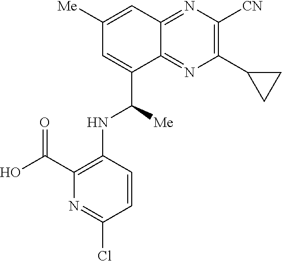 [SMILES: Cc1cc([C@@H](C)Nc2ccc(Cl)nc2C(=O)O)c2nc(C3CC3)c(C#N)nc2c1] | ¹H NMR (400 MHz, DMSO-d₆) δ = 8.51 (br d, J = 6.0 Hz, 1H), 7.81 (s, 1H), 7.78 (s, 1H), 7.25 (d, J = 8.80 Hz, 1H), 6.98 (d, J = 8.80 Hz, 1H), 5.52 (m, 1H), 2.66-2.57 (m, 1H), 2.07 (s, 3H), 1.64 (d, J = 6.80 Hz, 3H), 1.39-1.24 (m, 4H). LCMS [M + 1]⁺ = 408.1. |
| | (R)-6-chloro-3-((1-(2-cyano-3-cyclopropyl-7- | |
| | methylquinoxalin-5-yl)ethyl)amino)picolinic acid | |
| 45 | 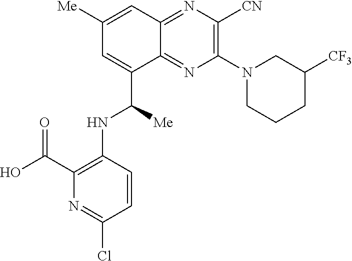 [SMILES: Cc1cc([C@@H](C)Nc2ccc(Cl)nc2C(=O)O)c2nc(N3CCCC(C(F)(F)F)C3)c(C#N)nc2c1] | ¹H NMR (400 MHz, DMSO-d₆) δ = 12.95 (br s, 1H), 8.56-8.44 (m, 1H), 7.72-7.64 (m, 2H), 7.32-7.25 (m, 1H), 7.10-6.95 (m, 1H), 5.51-5.39 (m, 1H), 4.50 (br t, J = 9.2 Hz, 1H), 4.42- 4.31 (m, 1H), 3.25-3.14 (m, 2H), 2.91-2.74 (m, 1H), 2.43 (d, J = 2.0 Hz, 3H), 2.07-2.00 (m, 1H), 1.93-1.84 (m, 1H), 1.76-1.62 (m, 5H). LCMS [M + Na]⁺ = 541.2. |
| | 6-chloro-3-(((1R)-1-(2-cyano-7-methyl-3-(3- | |
| | (trifluoromethyl)piperidin-1-yl)quinoxalin-5- | |
| | yl)ethyl)amino)picolinic acid | |
| 46A | 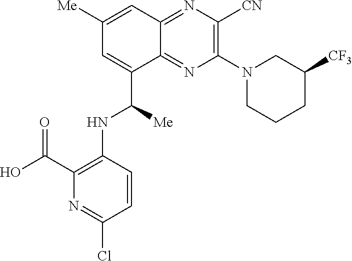 [SMILES: Cc1cc([C@@H](C)Nc2ccc(Cl)nc2C(=O)O)c2nc(N3CCC[C@H](C(F)(F)F)C3)c(C#N)nc2c1] | ¹H NMR (400 MHz, DMSO-d₆) δ = 12.98 (br s, 1H), 8.48 (br d, J = 6.8 Hz, 1H), 7.69 (s, 1H), 7.65 (d, J = 1.6 Hz, 1H), 7.28 (d, J = 9.2 Hz, 1H), 6.98 (d, J = 9.2 Hz, 1H), 5.48-5.43 (m, 1H), 4.52 (br d, J = 12.8 Hz, 1H), 4.36 (br d, J = 13.6 Hz, 1H), 3.28-3.13 (m, 2H), 2.91- 2.76 (m, 1H), 2.43 (s, 3H), 2.08- 2.00 (m, 1H), 1.92-1.84 (m, 1H), 1.76-1.60 (m, 5H). LCMS [M − 1]⁻ = 517.2. |
| | 6-chloro-3-(((R)-1-(2-cyano-7-methyl-3-((S)-3- | |
| | (trifluoromethyl)piperidin-1-yl)quinoxalin-5- | |
| | yl)ethyl)amino)picolinic acid | |
| 46B | 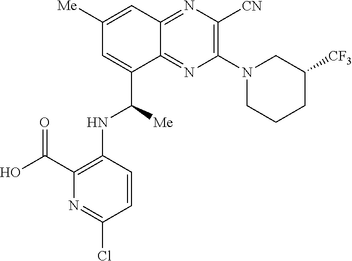 [SMILES: Cc1cc([C@@H](C)Nc2ccc(Cl)nc2C(=O)O)c2nc(N3CCC[C@@H](C(F)(F)F)C3)c(C#N)nc2c1] | ¹H NMR (400 MHz, DMSO-d₆) δ = 12.99 (br s, 1H), 8.52 (br s, 1H), 7.69 (s, 1H), 7.68 (s, 1H), 7.29 (d, J = 8.8 Hz, 1H), 7.06 (d, J = 8.8 Hz, 1H), 5.45 (m, 1H), 4.49 (br d, J = 12.4 Hz, 1H), 4.37 (br d, J = 13.6 Hz, 1H), 3.28-3.18 (m, 2H), 2.93- 2.74 (m, 1H), 2.43 (s, 3H), 2.08- 2.01 (m, 1H), 1.93-1.84 (m, 1H), 1.76-1.62 (m, 5H). LCMS [M + Na]⁺ = 541.1. |
| | 6-chloro-3-(((R)-1-(2-cyano-7-methyl-3-((R)-3- | |
| | (trifluoromethyl)piperidin-1-yl)quinoxalin-5- | |
| | yl)ethyl)amino)picolinic acid | |
| 47A | 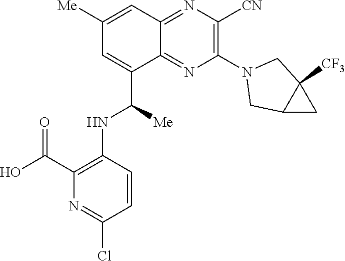 [SMILES: Cc1cc([C@@H](C)Nc2ccc(Cl)nc2C(=O)O)c2nc(N3CC4C[C@@]4(C(F)(F)F)C3)c(C#N)nc2c1] | |
| | 6-chloro-3-(((1R)-1-(2-cyano-7-methyl-3-((1S)-1- | |
| | (trifluoromethyl)-3-azabicyclo[3.1.0]hexan-3- | |
| | yl)quinoxalin-5-yl)ethyl)amino)picolinic acid | |
| 47B | 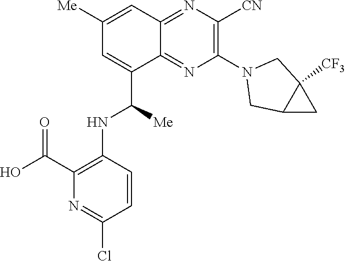 [SMILES: Cc1cc([C@@H](C)Nc2ccc(Cl)nc2C(=O)O)c2nc(N3CC4C[C@]4(C(F)(F)F)C3)c(C#N)nc2c1] | |
| | 6-chloro-3-(((1R)-1-(2-cyano-7-methyl-3-((1R)-1- | |
| | (trifluoromethyl)-3-azabicyclo[3.1.0]hexan-3- | |
| | yl)quinoxalin-5-yl)ethyl)amino)picolinic acid | |
| 48 | 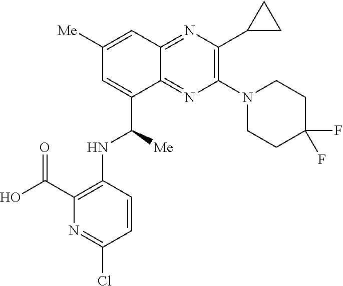 [SMILES: Cc1cc([C@@H](C)Nc2ccc(Cl)nc2C(=O)O)c2nc(N3CCC(F)(F)CC3)c(C3CC3)nc2c1] | ¹H NMR (400 MHz, DMSO-d6) δ = 8.52 (d, J = 8.4 Hz, 1H), 7.48 (d, J = 0.8 Hz, 1H), 7.39 (d, J = 1.6 Hz, 1H), 7.28 (d, J = 8.8 Hz, 1H), 7.05 (d, J = 9.2 Hz, 1H), 5.56-5.46 (m, 1H), 3.59- 3.58 (m, 4H), 2.45-2.40 (m, 1H), 2.38 (s, 3H), 2.30-2.18 (m, 4H), 1.65 (d, J = 6.8 Hz, 3H), 1.24-1.19 (m, 2H), 1.17- 1.15 (m, 2H). LCMS [M + 1]⁺ = 502.1. |
| | (R)-6-chloro-3-((1-(2-cyclopropyl-3-(4,4- | |
| | difluoropiperidin-1-yl)-7-methylquinoxalin-5- | |
| | yl)ethyl)amino)picolinic acid | |
| 49 | 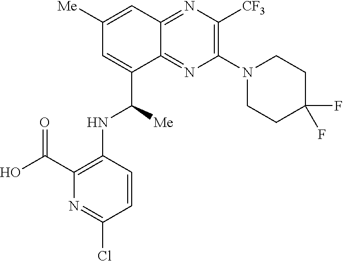 [SMILES: Cc1cc([C@@H](C)Nc2ccc(Cl)nc2C(=O)O)c2nc(N3CCC(F)(F)CC3)c(C(F)(F)F)nc2c1] | ¹H NMR (400 MHz, DMSO-d₆) δ = 12.99 (br s, 1H), 8.49 (br d, J = 7.2 Hz, 1H), 7.79 (s, 1H), 7.70 (d, J = 1.6 Hz, 1H), 7.27 (d, J = 8.8 Hz, 1H), 7.05 (d, J = 9.2 Hz, 1H), 5.53 (quin, J = 6.4 Hz, 1H), 3.57 (br t, J = 4.8 Hz, 4H), 2.46 (s, 3H), 2.26-2.13 (m, 4H), 1.68 (d, J = 6.8 Hz, 3H). LCMS [M + 23]⁺ = 552.1. |
| | (R)-6-chloro-3-((1-(3-(4,4-difluoropiperidin-1-yl)- | |
| | 7-methyl-2-(trifluoromethyl)quinoxalin-5- | |
| | yl)ethyl)amino)picolinic acid | |
| 50 | 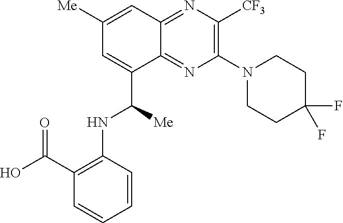 [SMILES: Cc1cc([C@@H](C)Nc2ccccc2C(=O)O)c2nc(N3CCC(F)(F)CC3)c(C(F)(F)F)nc2c1] | ¹H NMR (400 MHz, DMSO-d₆) δ = 12.70 (br s, 1H), 8.51 (br d, J = 4.4 Hz, 1H), 7.79 (dd, J = 1.6, 8.0 Hz, 1H), 7.77 (br s, 1H), 7.67 (d, J = 1.6 Hz, 1H), 7.19- 7.09 (m, 1H), 6.50 (t, J = 7.2 Hz, 1H), 6.44 (d, J = 8.4 Hz, 1H), 5.61-5.46 (m, 1H), 3.58 (br t, J = 5.6 Hz, 4H), 2.45 (s, 3H), 2.28- 2.13 (m, 4H), 1.64 (d, J = 6.8 Hz, 3H). LCMS [M + 1]⁺ = 495.3 |
| | (R)-2-((1-(3-(4,4-difluoropiperidin-1-yl)-7-methyl- | |
| | 2-(trifluoromethyl)quinoxalin-5- | |
| | yl)ethyl)amino)benzoic acid | |
| 51 | 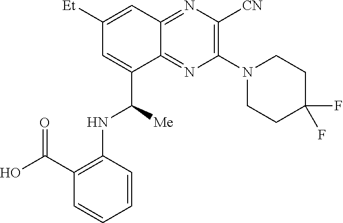 [SMILES: CCc1cc([C@@H](C)Nc2ccccc2C(=O)O)c2nc(N3CCC(F)(F)CC3)c(C#N)nc2c1] | ¹H NMR (400 MHz, CDCl₃) δ = 8.24 (s, 1H), 8.00 (d, J = 8.0 Hz, 1H), 7.69 (s, 1H), 7.66 (d, J = 1.6 Hz, 1H), 7.19 (dt, J = 1.6, 7.6 Hz, 1H), 6.59 (t, J = 7.6 Hz, 1H), 6.35 (d, J = 8.4 Hz, 1H), 5.67-5.46 (m, 1H), 4.04-3.81 (m, 4H), 2.78 (q, J = 7.6 Hz, 2H), 2.36-2.18 (m, 4H), 1.71 (d, J = 6.8 Hz, 3H), 1.26 (t, J = 7.6 Hz, 3H). LCMS [M + 1]⁺ = 466.2. |
| | (R)-2-((1-(2-cyano-3-(4,4-difluoropiperidin-1-yl)- | |
| | 7-ethylquinoxalin-5-yl)ethyl)amino)benzoic acid | |
| 52 | 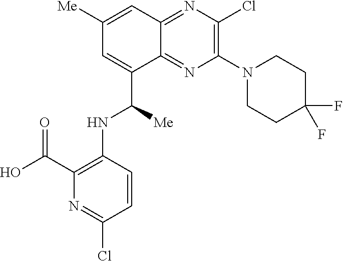 [SMILES: Cc1cc([C@@H](C)Nc2ccc(Cl)nc2C(=O)O)c2nc(N3CCC(F)(F)CC3)c(Cl)nc2c1] | |
| | (R)-6-chloro-3-((1-(2-chloro-3-(4,4- | |
| | difluoropiperidin-1-yl)-7-methylquinoxalin-5- | |
| | yl)ethyl)amino)picolinic acid | |
| 53 | 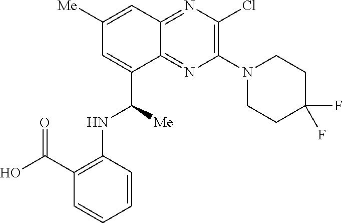 [SMILES: Cc1cc([C@@H](C)Nc2ccccc2C(=O)O)c2nc(N3CCC(F)(F)CC3)c(Cl)nc2c1] | ¹H NMR (400 MHz, DMSO-d₆) δ = 9.07-8.85 (m, 1H), 7.78 (dd, J = 1.6, 8.0 Hz, 1H), 7.57 (s, 1H), 7.53 (d, J = 1.6 Hz, 1H), 7.12-6.96 (m, 1H), 6.44 (t, J = 7.6 Hz, 1H), 6.33 (d, J = 8.4 Hz, 1H), 5.53-5.46 (m, 1H), 3.68 (br t, J = 4.8 Hz, 4H), 2.41 (s, 3H), 2.27-2.16 (m, 4H), 1.59 (d, J = 6.8 Hz, 3H). LCMS [M + 1]⁺ = 461.2. |
| | (R)-2-((1-(2-chloro-3-(4,4-difluoropiperidin-1-yl)- | |
| | 7-methylquinoxalin-5-yl)ethyl)amino)benzoic | |
| | acid | |
| 54 | 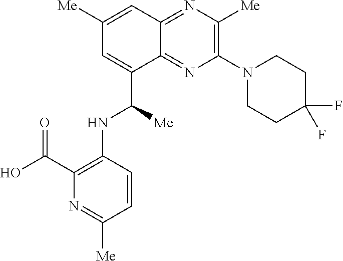 [SMILES: Cc1cc([C@@H](C)Nc2ccc(C)nc2C(=O)O)c2nc(N3CCC(F)(F)CC3)c(C)nc2c1] | ¹H NMR (400 MHz, DMSO-d₆) δ = 8.53 (br d, J = 7.2 Hz, 1H), 7.55 (s, 1H), 7.43 (d, J = 1.6 Hz, 1H), 7.18 (d, J = 8.8 Hz, 1H), 7.03 (d, J = 8.8 Hz, 1H), 5.53 (m, 1H), 3.51 (m, 4H), 2.67 (s, 3H), 2.40 (s, 3H), 2.33 (s, 3H), 2.25-2.15 (m, 4H), 1.65 (d, J = 6.8 Hz, 3H). LCMS [M + 1]⁺ = 456.1. |
| | (R)-3-((1-(3-(4,4-difluoropiperidin-1-yl)-2,7- | |
| | dimethylquinoxalin-5-yl)ethyl)amino)-6- | |
| | methylpicolinic acid | |
| 55 | 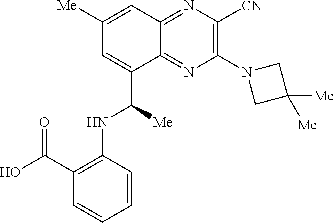 [SMILES: Cc1cc([C@@H](C)Nc2ccccc2C(=O)O)c2nc(N3CC(C)(C)C3)c(C#N)nc2c1] | ¹H NMR (400 MHz, CD₃OD) δ = 7.86 (dd, J = 1.6, 8.4 Hz, 1H), 7.53 (d, J = 2.0 Hz, 1H), 7.46 (s, 1H), 7.15-6.99 (m, 1H), 6.47 (t, J = 7.6 Hz, 1H), 6.38 (d, J = 8.8 Hz, 1H), 5.52 (q, J = 6.4 Hz, 1H), 4.25-4.04 (m, 4H), 2.36 (s, 3H), 1.63 (d, J = 6.8 Hz, 3H), 1.40 (s, 6H). LCMS [M + 1]⁺ = 416.1. |
| | (R)-2-((1-(2-cyano-3-(3,3-dimethylazetidin-1-yl)- | |
| | 7-methylquinoxalin-5-yl)ethyl)amino)benzoic | |
| | acid | |
| 56 | 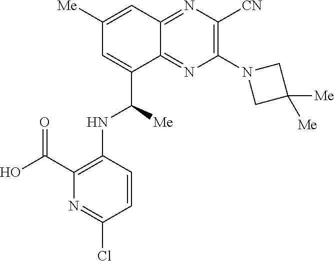 [SMILES: Cc1cc([C@@H](C)Nc2ccc(Cl)nc2C(=O)O)c2nc(N3CC(C)(C)C3)c(C#N)nc2c1] | ¹H NMR (400 MHz, CD₃OD) δ = 7.56 (d, J = 1.6 Hz, 1H), 7.53 (s, 1H), 7.19 (d, J = 8.8 Hz, 1H), 7.02 (d, J = 9.2 Hz, 1H), 5.48 (q, J = 6.8 Hz, 1H), 4.23-4.16 (m, 2H), 4.16-4.10 (m, 2H), 2.41 (s, 3H), 1.70 (d, J = 6.8 Hz, 3H), 1.40 (s, 6H). LCMS [M + 1]⁺ = 451.2. |
| | (R)-6-chloro-3-((1-(2-cyano-3-(3,3- | |
| | dimethylazetidin-1-yl)-7-methylquinoxalin-5- | |
| | yl)ethyl)amino)picolinic acid | |
| 57 | 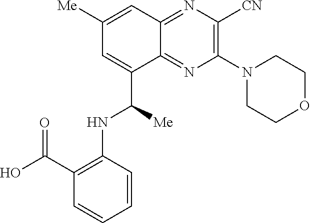 [SMILES: Cc1cc([C@@H](C)Nc2ccccc2C(=O)O)c2nc(N3CCOCC3)c(C#N)nc2c1] | ¹H NMR (400 MHz, CD₃OD) δ = 7.87 (dd, J = 1.6, 8.0 Hz, 1H), 7.65 (d, J = 1.6 Hz, 1H), 7.61 (s, 1H), 7.19-7.00 (m, 1H), 6.49 (t, J = 7.6 Hz, 1H), 6.41 (d, J = 8.4 Hz, 1H), 5.57 (q, J = 6.8 Hz, 1H), 3.96-3.87 (m, 4H), 3.83- 3.70 (m, 4H), 2.44 (s, 3H), 1.67 (d, J = 6.8 Hz, 3H). LCMS [M + 1]⁺ = 418.1. |
| | (R)-2-((1-(2-cyano-7-methyl-3- | |
| | morpholinoquinoxalin-5-yl)ethyl)amino)benzoic | |
| | acid | |
| 58 | 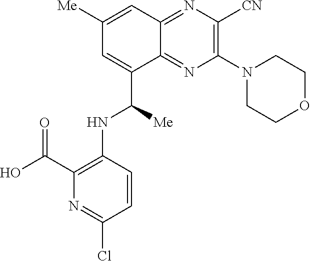 [SMILES: Cc1cc([C@@H](C)Nc2ccc(Cl)nc2C(=O)O)c2nc(N3CCOCC3)c(C#N)nc2c1] | ¹H NMR (400 MHz, CD₃OD) δ = 7.66 (d, J = 1.6 Hz, 1H), 7.63 (s, 1H), 7.19 (d, J = 9.2 Hz, 1H), 7.02 (d, J = 9.2 Hz, 1H), 5.53 (q, J = 6.8 Hz, 1H), 3.98-3.86 (m, 4H), 3.83-3.72 (m, 4H), 2.46 (s, 3H), 1.73 (d, J = 6.8 Hz, 3H). LCMS [M + 1]⁺ = 453.2. |
| | (R)-6-chloro-3-((1-(2-cyano-7-methyl-3- | |
| | morpholinoquinoxalin-5-yl)ethyl)amino)picolinic | |
| | acid | |
| 59 | 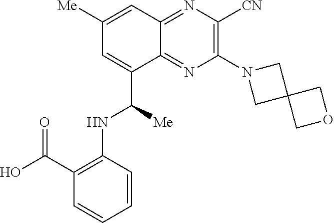 [SMILES: Cc1cc([C@@H](C)Nc2ccccc2C(=O)O)c2nc(N3CC4(COC4)C3)c(C#N)nc2c1] | ¹H NMR (400 MHz, CD₃OD) δ = 7.87 (dd, J = 1.2, 8.0 Hz, 1H), 7.57 (d, J = 1.6 Hz, 1H), 7.52 (s, 1H), 7.15-6.97 (m, 1H), 6.48 (t, J = 7.6 Hz, 1H), 6.38 (d, J = 8.4 Hz, 1H), 5.53 (q, J = 6.4 Hz, 1H), 4.90 (s, 4H), 4.68-4.60 (m, 4H), 2.39 (s, 3H), 1.65 (d, J = 6.4 Hz, 3H). LCMS [M + 1]⁺ = 430.2. |
| | (R)-2-((1-(2-cyano-7-methyl-3-(2-oxa-6- | |
| | azaspiro[3.3]heptan-6-yl)quinoxalin-5- | |
| | yl)ethyl)amino)benzoic acid | |
| 60 | 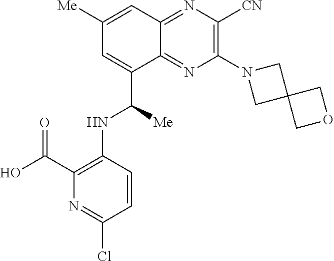 [SMILES: Cc1cc([C@@H](C)Nc2ccc(Cl)nc2C(=O)O)c2nc(N3CC4(COC4)C3)c(C#N)nc2c1] | ¹H NMR (400 MHz, CD₃OD) δ = 7.60 (d, J = 1.6 Hz, 1H), 7.57 (s, 1H), 7.20 (d, J = 9.2 Hz, 1H), 7.05 (d, J = 9.2 Hz, 1H), 5.48 (q, J = 6.8 Hz, 1H), 4.91 (s, 4H), 4.72-4.67 (m, 2H), 4.64-4.59 (m, 2H), 2.43 (s, 3H), 1.72 (d, J = 6.8 Hz, 3H). LCMS [M + 1]⁺ = 465.2. |
| | (R)-6-chloro-3-((1-(2-cyano-7-methyl-3-(2-oxa-6- | |
| | azaspiro[3.3]heptan-6-yl)quinoxalin-5- | |
| | yl)ethyl)amino)picolinic acid | |
| 61 | 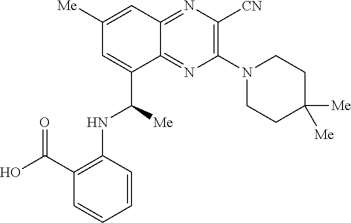 [SMILES: Cc1cc([C@@H](C)Nc2ccccc2C(=O)O)c2nc(N3CCC(C)(C)CC3)c(C#N)nc2c1] | ¹H NMR (400 MHz, CD₃OD) δ = 7.88 (dd, J = 1.2, 8.0 Hz, 1H), 7.61 (s, 1H), 7.58 (s, 1H), 7.16- 7.05 (m, 1H), 6.50 (t, J = 7.6 Hz, 1H), 6.38 (d, J = 8.4 Hz, 1H), 5.59 (q, J = 6.4 Hz, 1H), 3.78 (t, J = 5.6 Hz, 4H), 2.42 (s, 3H), 1.68-1.60 (m, 7H), 1.08 (s, 6H). LCMS [M + H]⁺: 444.2. |
| | (R)-2-((1-(2-cyano-3-(4,4-dimethylpiperidin-1-yl)- | |
| | 7-methylquinoxalin-5-yl)ethyl)amino)benzoic | |
| | acid | |
| 62 | 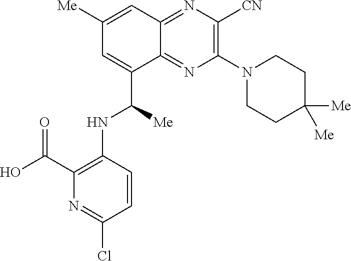 [SMILES: Cc1cc([C@@H](C)Nc2ccc(Cl)nc2C(=O)O)c2nc(N3CCC(C)(C)CC3)c(C#N)nc2c1] | ¹H NMR (400 MHz, CD₃OD) δ = 7.62 (br s, 2H), 7.20 (d, J = 8.8 Hz, 1H), 7.02 (d, J = 9.2 Hz, 1H), 5.54 (q, J = 6.4 Hz, 1H), 3.81-3.72 (m, 4H), 2.45 (s, 3H), 1.72 (d, J = 6.8 Hz, 3H), 1.62 (t, J = 5.6 Hz, 4H), 1.08 (s, 6H). LCMS [M + H]⁺: 479.1. |
| | (R)-6-chloro-3-((1-(2-cyano-3-(4,4- | |
| | dimethylpiperidin-1-yl)-7-methylquinoxalin-5- | |
| | yl)ethyl)amino)picolinic acid | |
| 63 | 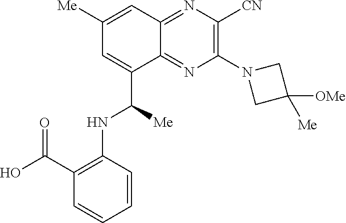 [SMILES: COC1(C)CN(c2nc3c([C@@H](C)Nc4ccccc4C(=O)O)cc(C)cc3nc2C#N)C1] | ¹H NMR (400 MHz, CD₃OD) δ = 7.89 (d, J = 7.6 Hz, 1H), 7.59 (s, 1H), 7.53 (br d, J = 3.2 Hz, 1H), 7.15-7.06 (m, 1H), 6.49 (br t, J = 7.2 Hz, 1H), 6.41 (d, J = 8.4 Hz, 1H), 5.55 (q, J = 6.4 Hz, 1H), 4.44-4.35 (m, 2H), 4.28 (dd, J = 10.8, 12.0 Hz, 2H), 3.36 (d, J = 0.8 Hz, 3H), 2.40 (s, 3H), 1.65 (d, J = 6.8 Hz, 3H), 1.62 (s, 3H). LCMS [M + 1] = 432.2. |
| | (R)-2-((1-(2-cyano-3-(3-methoxy-3- | |
| | methylazetidin-1-yl)-7-methylquinoxalin-5- | |
| | yl)ethyl)amino)benzoic acid | |
| 64 | 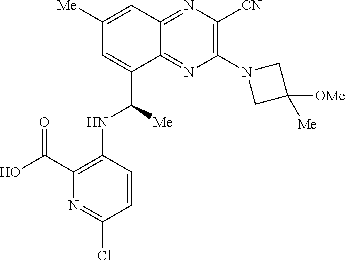 [SMILES: COC1(C)CN(c2nc3c([C@@H](C)Nc4ccc(Cl)nc4C(=O)O)cc(C)cc3nc2C#N)C1] | ¹H NMR (400 MHz, CD₃OD) δ = 7.61 (s, 1H), 7.58 (s, 1H), 7.22 (d, J = 9.2 Hz, 1H), 7.07 (d, J = 9.2 Hz, 1H), 5.51 (q, J = 6.8 Hz, 1H), 4.48-4.24 (m, 4H), 3.37 (s, 3H), 2.45 (s, 3H), 1.73 (d, J = 6.8 Hz, 3H), 1.62 (s, 3H). LCMS [M + 1]⁺ = 467.1. |
| | (R)-6-chloro-3-((1-(2-cyano-3-(3-methoxy-3- | |
| | methylazetidin-1-yl)-7-methylquinoxalin-5- | |
| | yl)ethyl)amino)picolinic acid | |
| 65 | 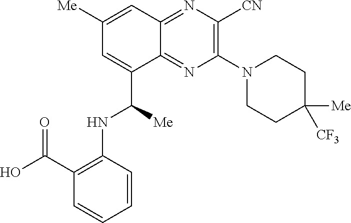 [SMILES: Cc1cc([C@@H](C)Nc2ccccc2C(=O)O)c2nc(N3CCC(C)(C(F)(F)F)CC3)c(C#N)nc2c1] | ¹H NMR (400 MHz, CD₃OD) δ = 7.90 (d, J = 1.6 Hz, 1H), 7.88 (d, J = 1.6 Hz, 1H), 7.91-7.87 (m, 1H), 7.65 (d, J = 1.8 Hz, 1H), 7.60 (d, J = 0.88 Hz, 1H), 7.10 (ddd, J = 1.6, 7.1, 8.6 Hz, 1H), 6.53-6.46 (m, 1H), 6.41 (d, J = 8.6 Hz, 1H), 5.60 (q, J = 6.8 Hz, 1H), 4.26 (br d, J = 13.6 Hz, 2H), 3.50-3.37 (m, 2H), 2.44 (s, 3H), 2.22-2.08 (m, 2H), 1.78-1.70 (m, 2H), 1.68 (d, J = 6.8 Hz, 3H), 1.38 (s, 3H). LCMS [M + H]⁺: 498.1. |
| | (R)-2-((1-(2-cyano-7-methyl-3-(4-methyl-4- | |
| | (trifluoromethyl)piperidin-1-yl)quinoxalin-5- | |
| | yl)ethyl)amino)benzoic acid | |
| 66 | 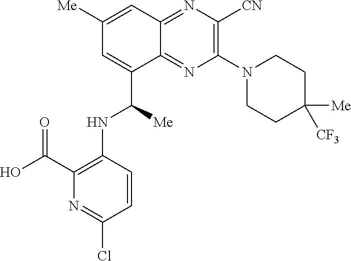 [SMILES: Cc1cc([C@@H](C)Nc2ccc(Cl)nc2C(=O)O)c2nc(N3CCC(C)(C(F)(F)F)CC3)c(C#N)nc2c1] | ¹H NMR (400 MHz, CD₃OD) δ = 7.66 (s, 1H), 7.63 (br s, 1H), 7.20 (d, J = 8.8 Hz, 1H), 7.04 (d, J = 9.2 Hz, 1H), 5.54 (q, J = 6.8 Hz, 1H), 4.24 (br t, J = 12.8 Hz, 2H), 3.50-3.35 (m, 2H), 2.46 (s, 3H), 2.12 (dt, J = 4.0, 12.8 Hz, 2H), 1.73 (br d, J = 6.8 Hz, 5H), 1.37 (s, 3H). LCMS [M + Na]⁺: 555.1. |
| | (R)-6-chloro-3-((1-(2-cyano-7-methyl-3-(4- | |
| | methyl-4-(trifluoromethyl)piperidin-1- | |
| | yl)quinoxalin-5-yl)ethyl)amino)picolinic acid | |
| 67 | 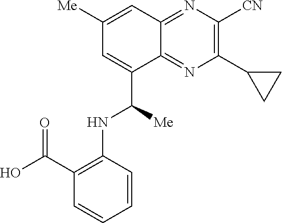 [SMILES: Cc1cc([C@@H](C)Nc2ccccc2C(=O)O)c2nc(C3CC3)c(C#N)nc2c1] | ¹H NMR (400 MHz, CD₃OD) δ = 7.88 (dd, J = 1.2, 8.0 Hz, 1H), 7.72 (s, 2H), 7.14-7.04 (m, 1H), 6.48 (t, J = 7.2 Hz, 1H), 6.32 (d, J = 8.4 Hz, 1H), 5.64 (q, J = 6.8 Hz, 1H), 2.72-2.64 (m, 1H), 2.48 (s, 3H), 1.64 (d, J = 6.8 Hz, 3H), 1.44-1.32 (m, 4H). LCMS [M + 1]⁺ = 373.1. |
| | (R)-2-((1-(2-cyano-3-cyclopropyl-7- | |
| | methylquinoxalin-5-yl)ethyl)amino)benzoic acid | |
| 68 | 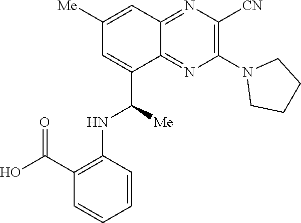 [SMILES: Cc1cc([C@@H](C)Nc2ccccc2C(=O)O)c2nc(N3CCCC3)c(C#N)nc2c1] | ¹H NMR (400 MHz, CD₃OD) δ = 7.87 (dd, J = 1.6, 8.0 Hz, 1H), 7.53 (d, J = 2.0 Hz, 1H), 7.49 (s, 1H), 7.08 (ddd, J = 1.6, 7.2, 8.8 Hz, 1H), 6.51-6.44 (m, 1H), 6.37 (d, J = 8.4 Hz, 1H), 5.54 (q, J = 6.8 Hz, 1H), 3.96-3.88 (m, 4H), 2.38 (s, 3H), 2.10 (td, J = 3.6, 6.8 Hz, 4H), 1.64 (d, J = 6.4 Hz, 3H). LCMS [M + H]⁺ = 402.2. |
| | (R)-2-((1-(2-cyano-7-methyl-3-(pyrrolidin-1- | |
| | yl)quinoxalin-5-yl)ethyl)amino)benzoic acid | |
| 69 | 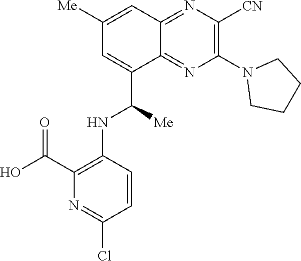 [SMILES: Cc1cc([C@@H](C)Nc2ccc(Cl)nc2C(=O)O)c2nc(N3CCCC3)c(C#N)nc2c1] | ¹H NMR (400 MHz, CD₃OD) δ = 7.53(s, 1H), δ = 7.50(s, 1H), 7.19 (d, J = 8.8 Hz, 1H), 7.01 (d, J = 8.8 Hz, 1H), 5.50 (q, J = 6.8 Hz, 1H), 4.05-3.77 (m, 4H), 2.40 (s, 3H), 2.10 (td, J = 3.6, 6.4 Hz, 4H), 1.71 (d, J = 6.4 Hz, 3H). LCMS [M + Na]⁺ = 459.2. |
| | (R)-6-chloro-3-((1-(2-cyano-7-methyl-3- | |
| | (pyrrolidin-1-yl)quinoxalin-5- | |
| | yl)ethyl)amino)picolinic acid | |
| 70 | 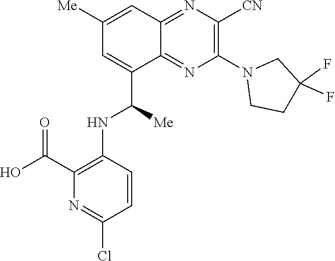 [SMILES: Cc1cc([C@@H](C)Nc2ccc(Cl)nc2C(=O)O)c2nc(N3CCC(F)(F)C3)c(C#N)nc2c1] | ¹H NMR (400 MHz, CD₃OD) δ = 7.60 (s, 1H), 7.57 (s, 1H), 7.19 (d, J = 9.2 Hz, 1H), 7.02 (d, J = 9.2 Hz, 1H), 5.50 (q, J = 6.8 Hz, 1H), 4.38-4.14 (m, 4H), 2.66-2.55 (m, 2H), 2.43 (s, 3H), 1.73 (d, J = 6.8 Hz, 3H). LCMS [M + Na]⁺ = 495.1. |
| | (R)-6-chloro-3-((1-(2-cyano-3-(3,3- | |
| | difluoropyrrolidin-1-yl)-7-methylquinoxalin-5- | |
| | yl)ethyl)amino)picolinic acid | |
| 71 | 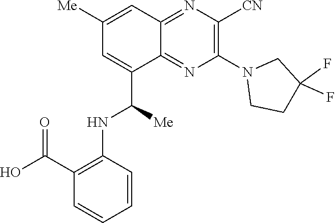 [SMILES: Cc1cc([C@@H](C)Nc2ccccc2C(=O)O)c2nc(N3CCC(F)(F)C3)c(C#N)nc2c1] | ¹H NMR (400 MHz, CD₃OD) δ = 7.87 (dd, J = 1.6, 8.0 Hz, 1H), 7.59 (d, J = 2.0 Hz, 1H), 7.56- 7.52 (m, 1H), 7.07 (ddd, J = 1.6, 7.2, 8.4 Hz, 1H), 6.50-6.44 (m, 1H), 6.38 (d, J = 8.4 Hz, 1H), 5.53 (q, J = 6.4 Hz, 1H), 4.36- 4.24 (m, 2H), 4.23-4.13 (m, 2H), 2.65-2.53 (m, 2H), 2.40 (s, 3H), 1.66 (d, J = 6.4 Hz, 3H). LCMS [M + H]⁺ = 438.2. |
| | (R)-2-((1-(2-cyano-3-(3,3-difluoropyrrolidin-1-yl)- | |
| | 7-methylquinoxalin-5-yl)ethyl)amino)benzoic | |
| | acid | |
| 72A and 72B | 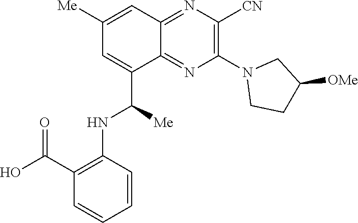 [SMILES: CO[C@H]1CCN(c2nc3c([C@@H](C)Nc4ccccc4C(=O)O)cc(C)cc3nc2C#N)C1] | Purified by SFC after saponification: First eluting final product (Example 72A): ¹H NMR (400 MHz, CD₃OD) δ = 7.76 (br d, J = 7.6 Hz, 1H), 7.54 (s, 1H), 7.38 (s, 1H), 6.82 (t, J = 7.6 Hz, 1H), 6.34 (t, J = 7.2 Hz, 1H), 6.14 (d, J = 8.4 Hz, 1H), 5.44 (q, J = 6.8 Hz, 1H), 4.12 (br d, J = 4.0 Hz, 1H), 3.98- 3.82 (m, 4H), 3.32 (s, 3H), 2.28 (s, 3H), 2.21-2.14 (m, 1H), 2.12-2.06 (m, 1H), 1.52 (d, J = 6.6 Hz, 3H). LCMS |
| | 2-(((R)-1-(2-cyano-3-((S)-3-methoxypyrrolidin-1- | [M + 1]⁺ = 432.0. |
| | yl)-7-methylquinoxalin-5-yl)ethyl)amino)benzoic | Second eluting final product |
| | acid | (Example 72B): ¹H NMR (400 |
| | OR | MHz, CD₃OD) δ = 7.88 (dd, J = |
| | 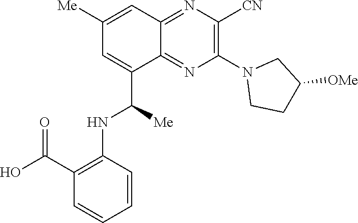 [SMILES: CO[C@@H]1CCN(c2nc3c([C@@H](C)Nc4ccccc4C(=O)O)cc(C)cc3nc2C#N)C1] | 1.6, 8.0 Hz, 1H), 7.56 (d, J = 1.6 Hz, 1H), 7.52 (s, 1H), 7.12 (ddd, J = 1.6, 7.2, 8.4 Hz, 1H), 6.52-6.48 (t, J = 7.2, 1H), 6.40 (d, J = 8.4 Hz, 1H), 5.58 (q, J = 6.8 Hz, 1H), 4.24-4.20 (m, 1H), 4.10-3.98 (m, 4H), 3.42 (s, 3H), 2.40 (s, 3H), 2.32-2.26 (m, 1H), 2.22-2.12 (m, 1H), 1.66 (d, J = 6.8 Hz, 3H). LCMS [M + 1]⁺ = 432.0. |
| | 2-(((R)-1-(2-cyano-3-((R)-3-methoxypyrrolidin-1- | |
| | yl)-7-methylquinoxalin-5-yl)ethyl)amino)benzoic | |
| | acid | |
| 73 | 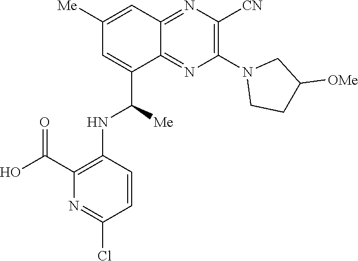 [SMILES: COC1CCN(c2nc3c([C@@H](C)Nc4ccc(Cl)nc4C(=O)O)cc(C)cc3nc2C#N)C1] | First eluting isomer: ¹H NMR (400 MHz, methanol- d₄) δ = 7.55 (s, 1H), 7.53 (s, 1H) 7.20 (d, J = 9.2 Hz, 1H), 7.02 (d, J = 8.8 Hz, 1H), 5.58-5.48 (m, 1H), 5.52 (q, J = 6.4 Hz, 1H), 4.24-4.16 (m, 1H), 4.08- 3.92 (m, 4H), 3.41 (s, 3H), 2.41 (s, 3H), 2.36-2.08 (m, 2H), 1.72 (d, J = 6.8 Hz, 3H). LCMS [M + 1]⁺ = 467.2. Second eluting isomer: ¹H NMR (400 MHz, methanol- d₄) δ = 7.56 (s, 1H), 7.54 (s, 1H), 7.20 (d, J = 8.8 Hz, 1H), 7.02 (d, J = 9.2 Hz, 1H), 5.51 |
| | 6-chloro-3-(((1R)-1-(2-cyano-3-(3- | (q, J = 6.8 Hz, 1H), 4.23-4.17 |
| | methoxypyrrolidin-1-yl)-7-methylquinoxalin-5- | (m, 1H), 4.07-3.92 (m, 4H), |
| | yl)ethyl)amino)picolinic acid | 3.42 (s, 3H), 2.42 (s, 3H), 2.33- |
| | | 2.23 (m, 1H), 2.21-2.08 (m, |
| | | 1H), 1.72 (d, J = 6.8 Hz, 3H). |
| | | LCMS [M + 1]⁺ = 467.3. |
| 74 | 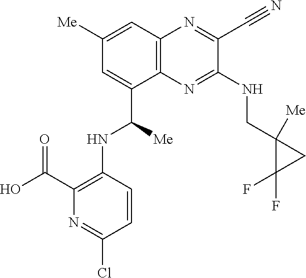 [SMILES: Cc1cc([C@@H](C)Nc2ccc(Cl)nc2C(=O)O)c2nc(NCC3(C)CC3(F)F)c(C#N)nc2c1] | ¹H NMR (400 MHz, CD₃OD) δ = 7.61-7.52 (m, 2H), 7.19 (dd, J = 7.2, 9.2 Hz, 1H), 6.94 (t, J = 8.8 Hz, 1H), 5.70-5.55 (m, 1H), 3.96-3.75 (m, 2H), 2.42 (s, 3H), 1.69 (dd, J = 1.6, 6.4 Hz, 3H), 1.59-1.48 (m, 1H), 1.35 (s, 3H), 1.27-1.13 (m, 1H). LCMS [M + Na]⁺ = 509.1. |
| | 6-chloro-3-(((1R)-1-(2-cyano-3-(((2,2-difluoro-1- | |
| | methylcyclopropyl)methyl)amino)-7- | |
| | methylquinoxalin-5-yl)ethyl)amino)picolinic acid | |
| 75A and 75B | 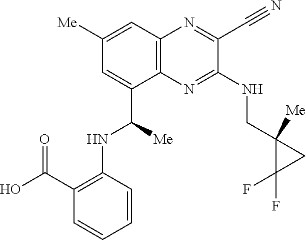 [SMILES: Cc1cc([C@@H](C)Nc2ccccc2C(=O)O)c2nc(NC[C@]3(C)CC3(F)F)c(C#N)nc2c1] | Purified by SFC prior to saponification: First eluting isomer leading to final compound 75A: ¹H NMR (400 MHz, CD₃OD) δ = 7.90 (dd, J = 1.6, 8.0 Hz, 1H), 7.60 (d, J = 2.0 Hz, 1H), 7.55 (d, J = 0.8 Hz, 1H), 7.12-7.06 (m, 1H), 6.51 (t, J = 7.2 Hz, 1H), 6.34 (d, J = 8.4 Hz, 1H), 5.66 (q, J = 6.8 Hz, 1H), 3.96-3.80 (m, 2H), 2.41 (s, 3H), 1.63 (d, J = 6.8 Hz, 3H), 1.60-1.52 (m, 1H), 1.36 (t, J = 2.0 Hz, 3H), 1.22-1.14 (m, 1H). LCMS [M + |
| | 2-(((R)-1-(2-cyano-3-((((S)-2,2-difluoro-1- | 1]⁺ = 452.1. |
| | methylcyclopropyl)methyl)amino)-7- | Second eluting isomer leading |
| | methylquinoxalin-5-yl)ethyl)amino)benzoic acid | to final compound 75B: ¹H |
| | AND | NMR (400 MHz, CD₃OD) δ = |
| | 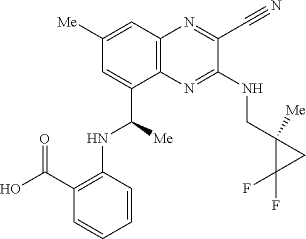 [SMILES: Cc1cc([C@@H](C)Nc2ccccc2C(=O)O)c2nc(NC[C@@]3(C)CC3(F)F)c(C#N)nc2c1] | 7.91 (dd, J = 1.6, 8.0 Hz, 1H), 7.59 (d, J = 1.6 Hz, 1H), 7.55 (s, 1H), 7.14-7.05 (m, 1H), 6.52 (t, J = 7.2 Hz, 1H), 6.34 (d, J = 8.4 Hz, 1H), 5.64 (q, J = 6.8 Hz, 1H), 3.98 (br d, J = 14.4 Hz, 1H), 3.77 (d, J = 14.8 Hz, 1H), 2.41 (s, 3H), 1.63 (d, J = 6.4 Hz, 3H), 1.61-1.53 (m, 1H), 1.37- 1.33 (m, 3H), 1.22-1.14 (m, 1H). LCMS [M + 1]⁺ = 452.1. |
| | 2-(((R)-1-(2-cyano-3-((((R)-2,2-difluoro-1- | |
| | methylcyclopropyl)methyl)amino)-7- | |
| | methylquinoxalin-5-yl)ethyl)amino)benzoic acid | |
| 76 | 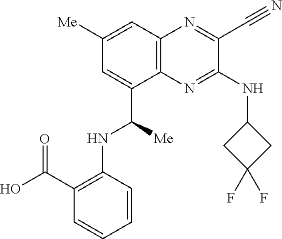 [SMILES: Cc1cc([C@@H](C)Nc2ccccc2C(=O)O)c2nc(NC3CC(F)(F)C3)c(C#N)nc2c1] | ¹H NMR (400 MHz, CD₃OD) δ = 7.88 (dd, J = 1.6, 8.0 Hz, 1H), 7.58 (d, J = 2.0 Hz, 1H), 7.53 (d, J = 0.8 Hz, 1H), 7.13-7.04 (m, 1H), 6.53-6.44 (m, 1H), 6.36 (d, J = 8.4 Hz, 1H), 5.59 (q, J = 6.8 Hz, 1H), 4.54-4.34 (m, 1H), 3.18-2.98 (m, 2H), 2.95-2.73 (m, 2H), 2.39 (s, 3H), 1.63 (d, J = 6.4 Hz, 3H). LCMS [M + 1]⁺ = 438.1. |
| | (R)-2-((1-(2-cyano-3-((3,3- | |
| | difluorocyclobutyl)amino)-7-methylquinoxalin-5- | |
| | yl)ethyl)amino)benzoic acid | |
| 77 | 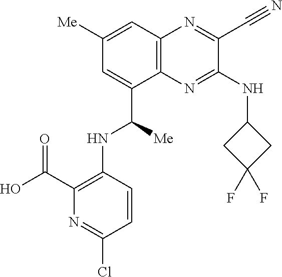 [SMILES: Cc1cc([C@@H](C)Nc2ccc(Cl)nc2C(=O)O)c2nc(NC3CC(F)(F)C3)c(C#N)nc2c1] | ¹H NMR (400 MHz, CD₃OD) δ = 7.60 (s, 1H), 7.59 (s, 1H), 7.20 (d, J = 8.8 Hz, 1H), 6.97 (d, J = 9.2 Hz, 1H), 5.61-5.55 (m 1H), 4.52-4.43 (m, 1H), 3.20-3.04 (m, 2H), 2.96-2.76 (m, 2H), 2.44 (s, 3H), 1.71 (d, J = 6.8 Hz, 3H). LCMS [M + Na]⁺ = 495.1. |
| | (R)-6-chloro-3-((1-(2-cyano-3-((3,3- | |
| | difluorocyclobutyl)amino)-7-methylquinoxalin-5- | |
| | yl)ethyl)amino)picolinic acid | |
| 78A | 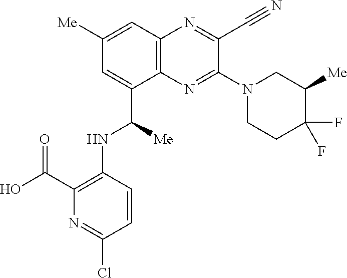 [SMILES: Cc1cc([C@@H](C)Nc2ccc(Cl)nc2C(=O)O)c2nc(N3CCC(F)(F)[C@H](C)C3)c(C#N)nc2c1] | ¹H NMR (400 MHz, CD₃OD) δ = 7.69-7.58 (m, 2H), 7.19 (d, J = 9.2 Hz, 1H), 7.02 (d, J = 9.2 Hz, 1H), 5.52 (q, J = 6.8 Hz, 1H), 4.38-4.20 (m, 2H), 3.56- 3.44 (m, 1H), 3.26-3.16 (m, 1H), 2.46 (s, 3H), 2.42-2.15 (m, 3H), 1.73 (d, J = 6.8 Hz, 3H), 1.15 (d, J = 6.8 Hz, 3H). LCMS [M + H]⁺: 501.2. |
| | 6-chloro-3-(((R)-1-(2-cyano-3-((R)-4,4-difluoro-3- | |
| | methylpiperidin-1-yl)-7-methylquinoxalin-5- | |
| | yl)ethyl)amino)picolinic acid | |
| 78B | 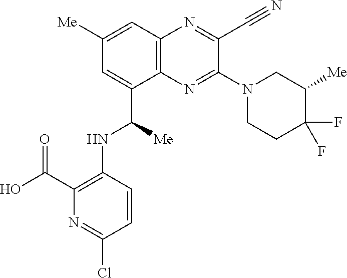 [SMILES: Cc1cc([C@@H](C)Nc2ccc(Cl)nc2C(=O)O)c2nc(N3CCC(F)(F)[C@@H](C)C3)c(C#N)nc2c1] | ¹H NMR (400 MHz, CD₃OD) δ = 7.72-7.58 (m, 2H), 7.20 (d, J = 9.2 Hz, 1H), 7.02 (d, J = 9.2 Hz, 1H), 5.54 (q, J = 6.8 Hz, 1H), 4.42-4.14 (m, 2H), 3.55- 3.41 (m, 1H), 3.26 (br dd, J = 11.2, 12.8 Hz, 1H), 2.46 (s, 3H), 2.43-2.10 (m, 3H), 1.73 (d, J = 6.8 Hz, 3H), 1.14 (d, J = 6.8 Hz, 3H). LCMS [M + 1]⁺ = 501.2. |
| | 6-chloro-3-(((R)-1-(2-cyano-3-((S)-4,4-difluoro-3- | |
| | methylpiperidin-1-yl)-7-methylquinoxalin-5- | |
| | yl)ethyl)amino)picolinic acid | |
| 79A | 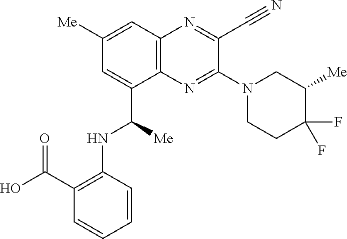 [SMILES: Cc1cc([C@@H](C)Nc2ccccc2C(=O)O)c2nc(N3CCC(F)(F)[C@@H](C)C3)c(C#N)nc2c1] | ¹H NMR (400 MHz, CD₃OD) δ = 7.88 (br d, J = 8.0 Hz, 1H), 7.65 (s, 1H), 7.60 (br d, J = 7.2 Hz, 1H), 7.09 (q, J = 6.8 Hz, 1H), 6.53-6.44 (m, 1H), 6.40 (d, J = 8.4 Hz, 1H), 5.57 (q, J = 6.8 Hz, 1H), 4.37-4.15 (m, 2H), 3.55-3.42 (m, 1H), 3.29- 3.18 (m, 1H), 2.44 (br d, J = 4.0 Hz, 3H), 2.40-2.08 (m, 3H), 1.67 (d, J = 6.8 Hz, 3H), 1.13 (d, J = 6.8 Hz, 3H). LCMS [M + 1]⁺ = 466.2. |
| | 2-(((R)-1-(2-cyano-3-((S)-4,4-difluoro-3- | |
| | methylpiperidin-1-yl)-7-methylquinoxalin-5- | |
| | yl)ethyl)amino)benzoic acid | |
| 79B | 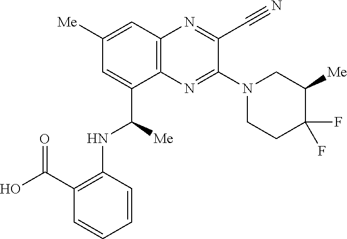 [SMILES: Cc1cc([C@@H](C)Nc2ccccc2C(=O)O)c2nc(N3CCC(F)(F)[C@H](C)C3)c(C#N)nc2c1] | ¹H NMR (400 MHz, CD₃OD) δ = 7.90 (dd, J = 1.2, 8.0 Hz, 1H), 7.68 (d, J = 1.6 Hz, 1H), 7.65 (s, 1H), 7.12 (dt, J = 1.6, 7.6 Hz, 1H), 6.52 (t, J = 7.6 Hz, 1H), 6.42 (d, J = 8.4 Hz, 1H), 5.63- 5.51 (m, 1H), 4.39-4.21 (m, 2H), 3.59-3.48 (m, 1H), 3.26 (br dd, J = 10.8, 12.8 Hz, 1H), 2.47 (s, 3H), 2.44-2.19 (m, 3H), 1.69 (d, J = 6.8 Hz, 3H), 1.16 (d, J = 6.8 Hz, 3H). LCMS [M + H]⁺: 466.2. |
| | 2-(((R)-1-(2-cyano-3-((R)-4,4-difluoro-3- | |
| | methylpiperidin-1-yl)-7-methylquinoxalin-5- | |
| | yl)ethyl)amino)benzoic acid | |
| 80 | 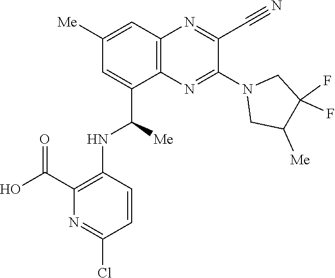 [SMILES: Cc1cc([C@@H](C)Nc2ccc(Cl)nc2C(=O)O)c2nc(N3CC(C)C(F)(F)C3)c(C#N)nc2c1] | ¹H NMR (400 MHz, CD₃OD) δ = 7.60 (s, 1H), 7.56 (br d, J = 9.6 Hz, 1H), 7.19 (dd, J = 3.2, 9.2 Hz, 1H), 7.04 (dd, J = 5.2, 9.2 Hz, 1H), 5.49 (q, J = 6.4 Hz, 1H), 4.45-4.18 (m, 3H), 3.82- 3.62 (m, 1H), 2.82 (tt, J = 8.0, 16.4 Hz, 1H), 2.43 (d, J = 2.4 Hz, 3H), 1.73 (d, J = 6.4 Hz, 3H), 1.25 (d, J = 6.4 Hz, 3H). LCMS [M + 1]⁺ = 509.1. |
| | 6-chloro-3-(((1R)-1-(2-cyano-3-(3,3-difluoro-4- | |
| | methylpyrrolidin-1-yl)-7-methylquinoxalin-5- | |
| | yl)ethyl)amino)picolinic acid | |
| 81 | 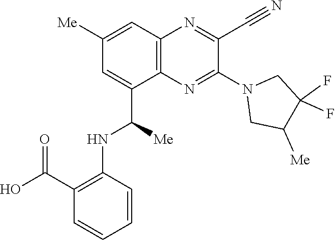 [SMILES: Cc1cc([C@@H](C)Nc2ccccc2C(=O)O)c2nc(N3CC(C)C(F)(F)C3)c(C#N)nc2c1] | |
| | 2-(((1R)-1-(2-cyano-3-(3,3-difluoro-4- | |
| | methylpyrrolidin-1-yl)-7-methylquinoxalin-5- | |
| | yl)ethyl)amino)benzoic acid | |
| 82 | 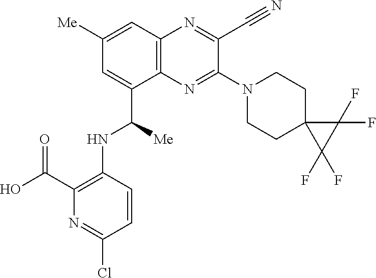 [SMILES: Cc1cc([C@@H](C)Nc2ccc(Cl)nc2C(=O)O)c2nc(N3CCC4(CC3)C(F)(F)C4(F)F)c(C#N)nc2c1] | ¹H NMR (400 MHz, CD₃OD) δ = 7.68-7.62 (m, 2H), 7.19 (d, J = 8.8 Hz, 1H), 7.03 (d, J = 8.8 Hz, 1H), 5.55 (q, J = 6.4 Hz, 1H), 3.88-3.79 (m, 4H), 2.46 (s, 3H), 2.10 (s, 4H), 1.73 (d, J = 6.4 Hz, 3H). LCMS [M + H]⁺ = 549.2. |
| | (R)-6-chloro-3-((1-(2-cyano-7-methyl-3-(1,1,2,2- | |
| | tetrafluoro-6-azaspiro[2.5]octan-6-yl)quinoxalin- | |
| | 5-yl)ethyl)amino)picolinic acid | |
| 83 | 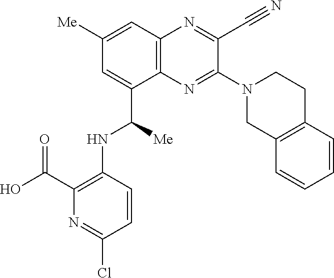 [SMILES: Cc1cc([C@@H](C)Nc2ccc(Cl)nc2C(=O)O)c2nc(N3CCc4ccccc4C3)c(C#N)nc2c1] | ¹H NMR (400 MHz, CD₃OD) δ = 7.63 (d, J = 3.6 Hz, 2H), 7.27 (br d, J = 4.4 Hz, 1H), 7.20 (d, J = 4.4 Hz, 3H), 7.15 (d, J = 8.8 Hz, 1H), 7.00 (d, J = 8.8 Hz, 1H), 5.56 (q, J = 6.4 Hz, 1H), 4.92 (br d, J = 4.8 Hz, 2H), 4.12 (t, J = 6.0 Hz, 2H), 3.12 (br t, J = 5.6 Hz, 2H), 2.45 (s, 3H), 1.72 (d, J = 6.8 Hz, 3H). LCMS [M + Na]⁺ = 521.1. |
| | (R)-6-chloro-3-((1-(2-cyano-3-(3,4- | |
| | dihydroisoquinolin-2(1H)-yl)-7-methylquinoxalin- | |
| | 5-yl)ethyl)amino)picolinic acid | |
| 84 | 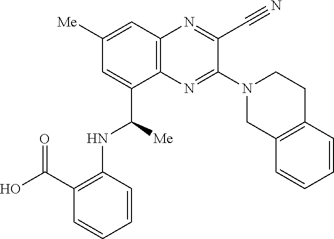 [SMILES: Cc1cc([C@@H](C)Nc2ccccc2C(=O)O)c2nc(N3CCc4ccccc4C3)c(C#N)nc2c1] | ¹H NMR (400 MHz, CD₃OD) δ = 7.88 (d, J = 7.6 Hz, 1H), 7.61 (d, J = 10.8 Hz, 2H), 7.26 (br d, J = 4.4 Hz, 1H), 7.19 (d, J = 4.4 Hz, 3H), 7.08 (t, J = 7.6 Hz, 1H), 6.49 (t, J = 7.6 Hz, 1H), 6.39 (d, J = 8.4 Hz, 1H), 5.60 (q, J = 6.8 Hz, 1H), 4.93 (d, J = 6.8 Hz, 2H), 4.12 (q, J = 6.4 Hz, 2H), 3.13 (q, J = 4.8 Hz, 2H), 2.43 (s, 3H), 1.66 (d, J = 6.8 Hz, 3H). LCMS [M + H]⁺ = 464.2. |
| | (R)-2-((1-(2-cyano-3-(3,4-dihydroisoquinolin- | |
| | 2(1H)-yl)-7-methylquinoxalin-5- | |
| | yl)ethyl)amino)benzoic acid | |
| 85 | 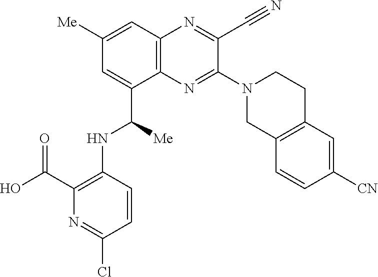 [SMILES: Cc1cc([C@@H](C)Nc2ccc(Cl)nc2C(=O)O)c2nc(N3CCc4cc(C#N)ccc4C3)c(C#N)nc2c1] | ¹H NMR (400 MHz, CD₃OD) δ = 7.67 (s, 2H), 7.62 (s, 1H), 7.59-7.54 (m, 1H), 7.53-7.49 (m, 1H), 7.15 (d, J = 8.8 Hz, 1H), 6.98 (d, J = 8.8 Hz, 1H), 5.51 (q, J = 6.4 Hz, 1H), 5.02- 4.95 (m, 2H), 4.14 (t, J = 6.0 Hz, 2H), 3.22-3.15 (m, 2H), 2.47 (s, 3H), 1.71 (d, J = 6.4 Hz, 3H). LCMS [M + 23]⁺ = 546.1. |
| | (R)-6-chloro-3-((1-(2-cyano-3-(6-cyano-3,4- | |
| | dihydroisoquinolin-2(1H)-yl)-7-methylquinoxalin- | |
| | 5-yl)ethyl)amino)picolinic acid | |
| 86 | 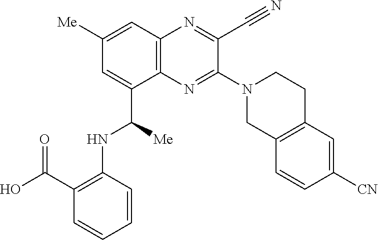 [SMILES: Cc1cc([C@@H](C)Nc2ccccc2C(=O)O)c2nc(N3CCc4cc(C#N)ccc4C3)c(C#N)nc2c1] | ¹H NMR (400 MHz, CD₃OD) δ = 7.88 (dd, J = 1.2, 8.0 Hz, 1H), 7.65 (s, 1H), 7.62 (br d, J = 10.0 Hz, 2H), 7.57-7.53 (m, 1H), 7.51-7.47 (m, 1H), 7.08 (t, J = 7.2 Hz, 1H), 6.50 (t, J = 7.6 Hz, 1H), 6.38 (d, J = 8.8 Hz, 1H), 5.55 (d, J = 6.8 Hz, 1H), 5.00- 4.92 (m, 2H), 4.21-4.07 (m, 2H), 3.18 (br d, J = 5.2 Hz, 2H), 2.45 (s, 3H), 1.65 (d, J = 6.8 Hz, 3H). LCMS [M + H]⁺ = 489.2. |
| | (R)-2-((1-(2-cyano-3-(6-cyano-3,4- | |
| | dihydroisoquinolin-2(1H)-yl)-7-methylquinoxalin- | |
| | 5-yl)ethyl)amino)benzoic acid | |
| 87 | 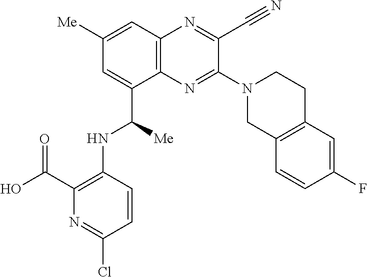 [SMILES: Cc1cc([C@@H](C)Nc2ccc(Cl)nc2C(=O)O)c2nc(N3CCc4cc(F)ccc4C3)c(C#N)nc2c1] | ¹H NMR (400 MHz, DMSO-d₆) δ = 8.85 (br s, 1H), 7.66 (s, 2H), 7.36 (dd, J = 8.8, 6.0 Hz, 1H), 7.18 (d, J = 8.8 Hz, 1H), 7.13- 7.02 (m, 2H), 6.94 (br d, J = 9.2 Hz, 1H), 5.52-5.37 (m, 1H), 4.87 (q, J = 16.4 Hz, 2H), 4.04 (t, J = 6.0 Hz, 2H), 3.08 (br t, J = 6.0 Hz, 2H), 2.42 (s, 3H), 1.62 (d, J = 6.8 Hz, 3H). |
| | (R)-6-chloro-3-((1-(2-cyano-3-(6-fluoro-3,4- | |
| | dihydroisoquinolin-2(1H)-yl)-7-methylquinoxalin- | |
| | 5-yl)ethyl)amino)picolinic acid | |
| 88 | 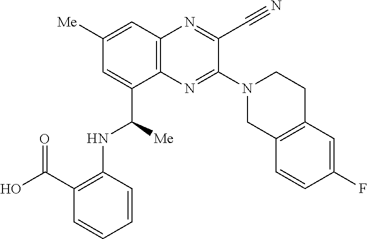 [SMILES: Cc1cc([C@@H](C)Nc2ccccc2C(=O)O)c2nc(N3CCc4cc(F)ccc4C3)c(C#N)nc2c1] | ¹H NMR (400 MHz, DMSO-d₆) δ = 8.54 (br s, 1H), 7.78 (dd, J = 1.6, 7.6 Hz, 1H), 7.65 (s, 1H), 7.64 (s, 1H), 7.35 (dd, J = 6.0, 8.4 Hz, 1H), 7.18-6.99 (m, 3H), 6.49 (t, J = 7.6 Hz, 1H), 6.43 (d, J = 8.0 Hz, 1H), 5.51- 5.48 (m, 1H), 4.97-4.79 (m, 2H), 4.07-4.03 (m, 2H), 3.15- 3.05 (m, 2H), 2.42 (s, 3H), 1.61 (d, J = 6.8 Hz, 3H). LCMS [M + Na]⁺ = 504.2. |
| | (R)-2-((1-(2-cyano-3-(6-fluoro-3,4- | |
| | dihydroisoquinolin-2(1H)-yl)-7-methylquinoxalin- | |
| | 5-yl)ethyl)amino)benzoic acid | |
| 89 | 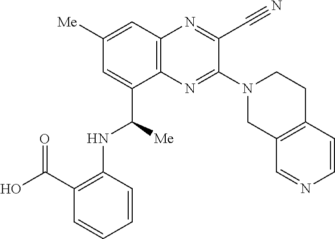 [SMILES: Cc1cc([C@@H](C)Nc2ccccc2C(=O)O)c2nc(N3CCc4ccncc4C3)c(C#N)nc2c1] | ¹H NMR (400 MHz, DMSO-d₆) δ = 8.59 (br s, 1H), 8.50 (s, 1H), 8.35 (d, J = 5.2 Hz, 1H), 7.78 (d, J = 7.6 Hz, 1H), 7.66 (s, 1H), 7.65 (s, 1H), 7.25 (d, J = 4.8 Hz, 1H), 7.12 (br t, J = 7.6 Hz, 1H), 6.49 (t, J = 7.6 Hz, 1H), 6.41 (br d, J = 8.4 Hz, 1H), 5.49-5.48 (m, 1H), 4.99-4.88 (m, 2H), 4.11-4.03 (m, 2H), 3.09 (br s, 2H), 2.42 (s, 3H), 1.59 (d, J = 6.8 Hz, 3H). LCMS [M + 1]⁺ = 465.2. |
| | (R)-2-((1-(2-cyano-3-(3,4-dihydro-2,7- | |
| | naphthyridin-2(1H)-yl)-7-methylquinoxalin-5- | |
| | yl)ethyl)amino)benzoic acid | |
| 90 | 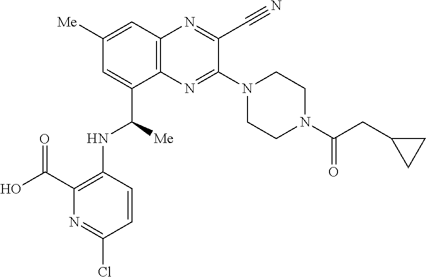 [SMILES: Cc1cc([C@@H](C)Nc2ccc(Cl)nc2C(=O)O)c2nc(N3CCN(C(=O)CC4CC4)CC3)c(C#N)nc2c1] | ¹H NMR (400 MHz, CD₃OD) δ = 7.67 (d, J = 4.8 Hz, 2H), 7.20 (d, J = 8.8 Hz, 1H), 7.05 (d, J = 8.8 Hz, 1H), 5.53 (q, J = 6.4 Hz, 1H), 3.90-3.76 (m, 8H), 2.47 (s, 3H), 2.43 (d, J = 6.8 Hz, 2H), 1.74 (d, J = 6.4 Hz, 3H), 1.12- 1.01 (m, 1H), 0.61-0.54 (m, 2H), 0.23 (q, J = 4.8 Hz, 2H). LCMS [M + H]⁺ = 534.3. |
| | (R)-6-chloro-3-((1-(2-cyano-3-(4-(2- | |
| | cyclopropylacetyl)piperazin-1-yl)-7- | |
| | methylquinoxalin-5-yl)ethyl)amino)picolinic acid | |
| 91 | 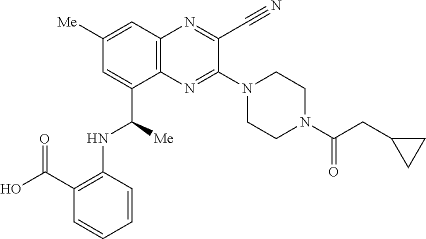 [SMILES: Cc1cc([C@@H](C)Nc2ccccc2C(=O)O)c2nc(N3CCN(C(=O)CC4CC4)CC3)c(C#N)nc2c1] | ¹H NMR (400 MHz, CD₃OD) δ = 7.91-7.84 (m, 1H), 7.67(s, 1H), 7.63 (s, 1H), 7.14-7.06 (m, 1H), 6.49 (t, J = 7.6 Hz, 1H), 6.41 (d, J = 8.4 Hz, 1H), 5.57 (q, J = 6.4 Hz, 1H), 3.89-3.76 (m, 8H), 2.45 (s, 3H), 2.42 (d, J = 6.8 Hz, 2H), 1.68 (d, J = 6.8 Hz, 3H), 1.11-1.00 (m, 1H), 0.60-0.53 (m, 2H), 0.26-0.19 (m, 2H). LCMS [M + H]⁺ = 499.3. |
| | (R)-2-((1-(2-cyano-3-(4-(2- | |
| | cyclopropylacetyl)piperazin-1-yl)-7- | |
| | methylquinoxalin-5-yl)ethyl)amino)benzoic acid | |
| 92A and 92B | 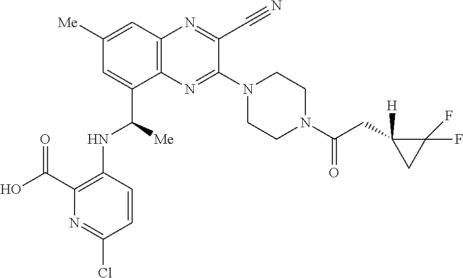 [SMILES: Cc1cc([C@@H](C)Nc2ccc(Cl)nc2C(=O)O)c2nc(N3CCN(C(=O)C[C@H]4CC4(F)F)CC3)c(C#N)nc2c1] | Purified by SFC prior to saponification: First eluting isomer leading to final compound 92A: ¹H NMR (400 MHz, CD₃OD) δ = 7.70- 7.65 (m, 2H), 7.21 (d, J = 8.8 Hz, 1H), 7.05 (d, J = 9.2 Hz, 1H), 5.53 (q, J = 6.8 Hz, 1H), 3.95-3.76 (m, 8H), 2.70 (br d, J = 7.2 Hz, 2H), 2.48 (s, 3H), 2.02-1.85 (m, 1H), 1.74 (d, J = 6.8 Hz, 3H), 1.56 (m, 1H), 1.21- 1.08 (m, 1H). LCMS [M + Na]⁺ = 592.2. Second eluting isomer leading to final compound 92B: ¹H NMR (400 MHz, CD₃OD) δ = |
| | 6-chloro-3-(((R)-1-(2-cyano-3-(4-(2-((S)-2,2- | 7.67 (s, 2H), 7.20 (d, J = 8.8 Hz, |
| | difluorocyclopropyl)acetyl)piperazin-1-yl)-7- | 1H), 7.05 (d, J = 9.2 Hz, 1H), |
| | methylquinoxalin-5-yl)ethyl)amino)picolinic acid | 5.53 (q, J = 6.8 Hz, 1H), 3.91- |
| | AND | 3.74 (m, 8H), 2.70 (br d, J = 7.2 |
| | 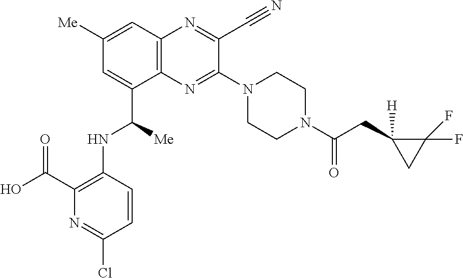 [SMILES: Cc1cc([C@@H](C)Nc2ccc(Cl)nc2C(=O)O)c2nc(N3CCN(C(=O)C[C@@H]4CC4(F)F)CC3)c(C#N)nc2c1] | Hz, 2H), 2.47 (s, 3H), 1.93 (m, 1H), 1.74 (d, J = 6.8 Hz, 3H), 1.56 (m, 1H), 1.19-1.07 (m, 1H). LCMS [M + Na]⁺ = 592.2. |
| | 6-chloro-3-(((R)-1-(2-cyano-3-(4-(2-((R)-2,2- | |
| | difluorocyclopropyl)acetyl)piperazin-1-yl)-7- | |
| | methylquinoxalin-5-yl)ethyl)amino)picolinic acid | |
| 93 | 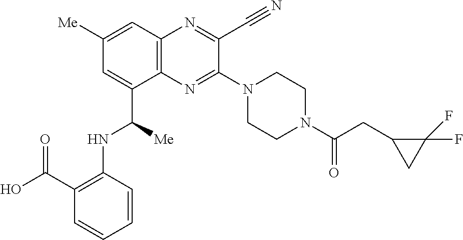 [SMILES: Cc1cc([C@@H](C)Nc2ccccc2C(=O)O)c2nc(N3CCN(C(=O)CC4CC4(F)F)CC3)c(C#N)nc2c1] | |
| | 2-(((1R)-1-(2-cyano-3-(4-(2-(2,2- | |
| | difluorocyclopropyl)acetyl)piperazin-1-yl)-7- | |
| | methylquinoxalin-5-yl)ethyl)amino)benzoic acid | |
| 94 | 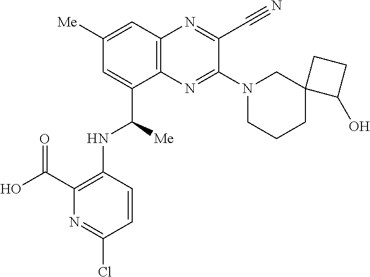 [SMILES: Cc1cc([C@@H](C)Nc2ccc(Cl)nc2C(=O)O)c2nc(N3CCCC4(CCC4O)C3)c(C#N)nc2c1] | |
| | 6-chloro-3-(((1R)-1-(2-cyano-3-(1-hydroxy-6- | |
| | azaspiro[3.5]nonan-6-yl)-7-methylquinoxalin-5- | |
| | yl)ethyl)amino)picolinic acid | |
| 95A and 95B | 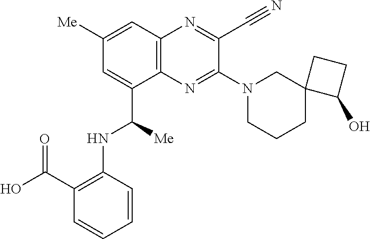 [SMILES: Cc1cc([C@@H](C)Nc2ccccc2C(=O)O)c2nc(N3CCCC4(CC[C@H]4O)C3)c(C#N)nc2c1] | Purified by SFC after saponification: First eluting isomer 95A: ¹H NMR (400 MHz, DMSO-d₆) δ = 12.68 (br s, 1H), 8.46 (br s, 1H), 7.79 (dd, J = 1.2, 8.0 Hz, 1H), 7.69-7.53 (m, 2H), 7.25-7.07 (m, 1H), 6.50 (t, J = 7.6 Hz, 1H), 6.41 (t, J = 8.0 Hz, 1H), 5.50 (br s, 1H), 4.94 (br s, 1H), 3.97- 3.84 (m, 2H), 3.79 (br dd, J = 6.0, 12.8 Hz, 1H), 3.48 (br dd, J = 2.4, 12.8 Hz, 1H), 3.41-3.40 (m, 1H), 2.41 (s, 3H), 2.18- 2.04 (m, 1H), 1.92-1.65 (m, |
| | 2-(((1R)-1-(2-cyano-3-((1R)-1-hydroxy-6- | 5H), 1.61 (br d, J = 5.6 Hz, 3H), |
| | azaspiro[3.5]nonan-6-yl)-7-methylquinoxalin-5- | 1.53-1.38 (m, 2H). LCMS |
| | yl)ethyl)amino)benzoic acid | [M + 1]⁺ = 472.2. |
| | AND | Second eluting isomer 95B: ¹H |
| | 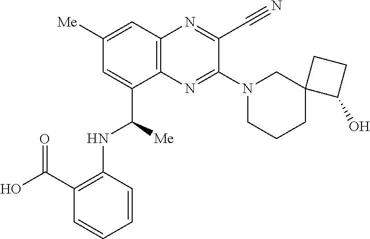 [SMILES: Cc1cc([C@@H](C)Nc2ccccc2C(=O)O)c2nc(N3CCCC4(CC[C@@H]4O)C3)c(C#N)nc2c1] | NMR (400 MHz, DMSO-d₆) δ = 12.69 (br s, 1H), 8.47 (br s, 1H), 7.79 (dd, J = 1.6, 8.0 Hz, 1H), 7.60 (d, J = 0.8 Hz, 1H), 7.57 (d, J = 2.0 Hz, 1H), 7.21-7.02 (m, 1H), 6.49 (t, J = 7.6 Hz, 1H), 6.38 (dd, J = 8.4, 14.8 Hz, 1H), 5.52 (br d, J = 6.4 Hz, 1H), 5.20- 4.72 (m, 1H), 4.27 (br dd, J = 9.6, 12.8 Hz, 1H), 4.03-3.88 (m, 1H), 3.80 (br t, J = 7.2 Hz, 1H), 3.54 (br dd, J = 3.2, 13.2 Hz, 1H), 3.44-3.40 (m, 1H), 2.39 (s, 3H), 2.18-2.06 (m, 1H), 1.96-1.84 (m, 1H), 1.83- |
| | 2-(((1R)-1-(2-cyano-3-((1S)-1-hydroxy-6- | 1.65 (m, 4H), 1.60 (br dd, J = |
| | azaspiro[3.5]nonan-6-yl)-7-methylquinoxalin-5- | 2.4, 6.4 Hz, 4H), 1.26-1.19 (m, |
| | yl)ethyl)amino)benzoic acid | 1H). LCMS [M + 1]⁺ = 472.2. |
| 96A And 96B | 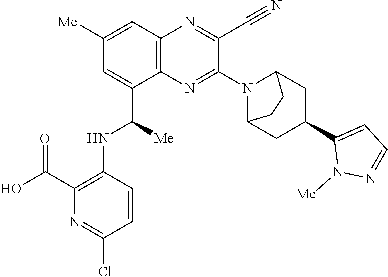 | Purified by SFC prior to saponification: First eluting isomer leading to 96A: ¹H NMR (400 MHz, CD₃OD) δ = 7.62 (d, J = 1.6 Hz, 1H), 7.57 (s, 1H), 7.31 (d, J = 2.0 Hz, 1H), 7.14 (d, J = 9.2 Hz, 1H), 6.99 (d, J = 8.8 Hz, 1H), 6.02 (d, J = 2.0 Hz, 1H), 5.55 (q, J = 6.8 Hz, 1H), 5.18-5.06 (m, 2H), 3.86 (s, 3H), 3.53- 3.42 (m, 1H), 2.43 (s, 3H), 2.24- 1.95 (m, 8H), 1.69 (d, J = 6.8 Hz, 3H). LCMS [M + 1]⁺ = 557.3. Second eluting isomer leading to 96B: ¹H NMR (400 MHz, methanol-d₄) δ = 7.62 (s, 1H), |
| | 6-chloro-3-(((1R)-1-(2-cyano-7-methyl-3-(3-(1- | 7.59 (s, 1H), 7.36 (d, J = 2.0 Hz, |
| | methyl-1H-pyrazol-5-yl)-8- | 1H), 7.16 (d, J = 8.8 Hz, 1H), |
| | azabicyclo[3.2.1]octan-8-yl)quinoxalin-5- | 7.00 (d, J = 9.2 Hz, 1H), 6.25 |
| | yl)ethyl)amino)picolinic acid | (d, J = 1.6 Hz, 1H), 5.53 (q, J = |
| | AND | 6.8 Hz, 1H), 5.15-5.06 (m, |
| | 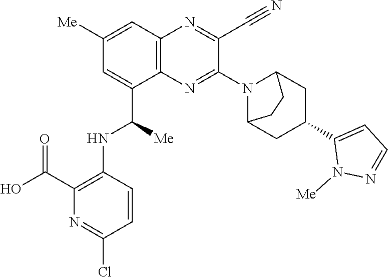 | 1H), 5.05-4.99 (m, 1H), 3.74 (s, 3H), 3.16-3.07 (m, 1H), 2.84-2.71 (m, 2H), 2.44 (s, 3H), 2.22-2.09 (m, 2H), 1.97- 1.84 (m, 2H), 1.82-1.73 (m, 2H), 1.72 (d, J = 6.4 Hz, 3H). LCMS [M + 1]⁺ = 557.3. |
| | 6-chloro-3-(((1R)-1-(2-cyano-7-methyl-3-(3-(1- | |
| | methyl-1H-pyrazol-5-yl)-8- | |
| | azabicyclo[3.2.1]octan-8-yl)quinoxalin-5- | |
| | yl)ethyl)amino)picolinic acid | |
| 97A and 97B | 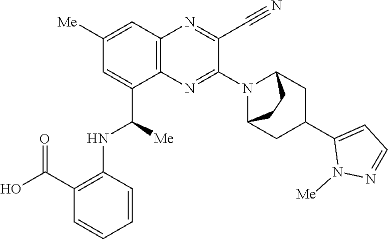 [SMILES: Cc1cc([C@@H](C)Nc2ccccc2C(=O)O)c2nc(N3[C@@H]4CC[C@H]3CC(c3ccnn3C)C4)c(C#N)nc2c1] | Purified by SFC prior to saponification: First eluting isomer leading to 97A: ¹H NMR (400 MHz, CD₃OD) δ = 7.86 (d, J = 8.0 Hz, 1H), 7.59-7.55 (m, 2H), 7.31 (d, J = 1.6 Hz, 1H), 7.07-6.99 (m, 1H), 6.47 (t, J = 7.2 Hz, 1H), 6.38 (d, J = 8.4 Hz, 1H), 6.02 (d, J = 1.6 Hz, 1H), 5.68-5.52 (m, 1H), 5.14-5.05 (m, 2H), 3.85 (s, 3H), 3.51-3.39 (m, 1H), 2.40 (s, 3H), 2.25-1.93 (m, 8H), 1.63 (d, J = 6.8 Hz, 3H). LCMS [M + Na]⁺ = 522.2. |
| | 2-(((1R)-1-(2-cyano-7-methyl-3-((1R,5S)-3-(1- | Second eluting isomer leading |
| | methyl-1H-pyrazol-5-yl)-8- | to 97B: ¹H NMR (400 MHz, |
| | azabicyclo[3.2.1]octan-8-yl)quinoxalin-5- | DMSO-d₆) δ = 8.47 (br s, 1H), |
| | yl)ethyl)amino)benzoic acid | 7.78 (dd, J = 1.6, 8.0 Hz, 1H), |
| | AND | 7.61 (d, J = 8.4 Hz, 2H), 7.29 |
| | 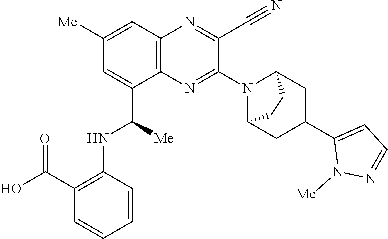 [SMILES: Cc1cc([C@@H](C)Nc2ccccc2C(=O)O)c2nc(N3[C@@H]4CC[C@H]3CC(c3ccnn3C)C4)c(C#N)nc2c1] | (d, J = 2.0 Hz, 1H), 7.11 (t, J = 7.2 Hz, 1H), 6.48 (t, J = 7.2 Hz, 1H), 6.40 (d, J = 8.4 Hz, 1H), 6.22 (d, J = 1.6 Hz, 1H), 5.52- 5.40 (m, 1H), 5.07-4.95 (m, 2H), 3.65 (s, 3H), 3.07-3.03 (m, 1H), 2.70-2.62 (m, 2H), 2.40 (s, 3H), 2.14-2.06 (m, 2H), 1.87-1.79 (m, 2H), 1.75- 1.67 (m, 2H), 1.60 (d, J = 6.4 Hz, 3H). LCMS [M + 23]⁺ = 522.2. |
| | 2-(((1R)-1-(2-cyano-7-methyl-3-((1R,5S)-3-(1- | |
| | methyl-1H-pyrazol-5-yl)-8- | |
| | azabicyclo[3.2.1]octan-8-yl)quinoxalin-5- | |
| | yl)ethyl)amino)benzoic acid | |
| 98A and 98B | 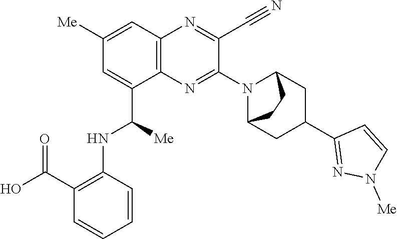 [SMILES: Cc1cc([C@@H](C)Nc2ccccc2C(=O)O)c2nc(N3[C@@H]4CC[C@H]3CC(c3ccn(C)n3)C4)c(C#N)nc2c1] | Purified by SFC prior to saponification: First eluting isomer leading to 98A: ¹H NMR (400 MHz, CDCl₃) δ = 8.32-8.05 (m, 1H), 7.96 (d, J = 8.0 Hz, 1H), 7.57 (br s, 2H), 7.24 (d, J = 2.0 Hz, 1H), 7.18-7.11 (m, 1H), 6.56 (t, J = 6.8 Hz, 1H), 6.44-6.35 (m, 1H), 6.01 (d, J = 2.4 Hz, 1H), 5.54 (m, 1H), 5.22-5.15 (m, 2H), 3.85 (s, 3H), 3.48- 3.35 (m, 1H), 2.43 (s, 3H), 2.22-2.11 (m, 4H), 2.18-2.00 (m, 4H), 1.67 (d, J = 6.8 Hz, |
| | 2-(((1R)-1-(2-cyano-7-methyl-3-((1R,5S)-3-(1- | 3H). LCMS [M + 1]⁺ = 522.2. |
| | methyl-1H-pyrazol-3-yl)-8- | Second eluting isomer leading |
| | azabicyclo[3.2.1]octan-8-yl)quinoxalin-5- | to 98B: ¹H NMR (400 MHz, |
| | yl)ethyl)amino)benzoic acid | CDCl₃) δ = 8.36-8.13 (m, 1H), |
| | AND | 7.98 (d, J = 7.6 Hz, 1H), 7.56 |
| | 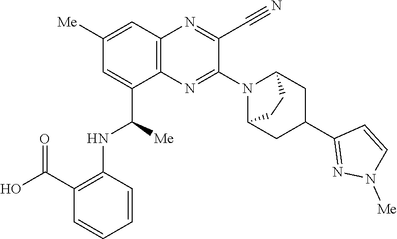 [SMILES: Cc1cc([C@@H](C)Nc2ccccc2C(=O)O)c2nc(N3[C@@H]4CC[C@H]3CC(c3ccn(C)n3)C4)c(C#N)nc2c1] | (s, 1H), 7.55 (s, 1H), 7.29 (d, J = 2.0 Hz, 1H), 7.17 (t, J = 7.6 Hz, 1H), 6.56 (t, J = 7.2 Hz, 1H), 6.39 (br d, J = 8.4 Hz, 1H), 6.16 (d, J = 2.4 Hz, 1H), 5.57-5.47 (m, 1H), 5.21-5.06 (m, 2H), 3.89 (s, 3H), 3.15- 3.04 (m, 1H), 2.67-2.56 (m, 2H), 2.43 (s, 3H), 2.39-2.29 (m, 2H), 2.02-1.90 (m, 2H), 1.89-1.81 (m, 2H), 1.68 (d, J = 6.4 Hz, 3H). LCMS [M + 1]⁺ = 522.2. |
| | 2-(((1R)-1-(2-cyano-7-methyl-3-((1R,5S)-3-(1- | |
| | methyl-1H-pyrazol-3-yl)-8- | |
| | azabicyclo[3.2.1]octan-8-yl)quinoxalin-5- | |
| | yl)ethyl)amino)benzoic acid | |
| 99A and 99B | 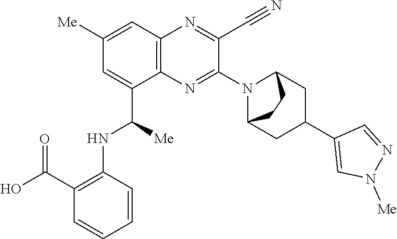 [SMILES: Cc1cc([C@@H](C)Nc2ccccc2C(=O)O)c2nc(N3[C@@H]4CC[C@H]3CC(c3cnn(C)c3)C4)c(C#N)nc2c1] | Purified by SFC prior to saponification: First eluting isomer leading to 99A: ¹H NMR (400 MHz, DMSO-d₆) δ = 12.68 (br s, 1H), 8.43 (br d, J = 6.4 Hz, 1H), 7.78 (dd, J = 1.6, 8.0 Hz, 1H), 7.61 (s, 1H), 7.59 (s, 1H), 7.43 (s, 1H), 7.24 (s, 1H), 7.12- 7.04 (m, 1H), 6.49 (t, J = 7.6 Hz, 1H), 6.40 (d, J = 8.4 Hz, 1H), 5.56-5.44 (m, 1H), 5.06 (br s, 1H), 5.00 (br s, 1H), 3.72 (s, 3H), 3.23-3.10 (m, 1H), 2.40 (s, 3H), 2.16-2.05 (m, |
| | 2-(((1R)-1-(2-cyano-7-methyl-3-((1R,5S)-3-(1- | 2H), 2.01-1.82 (m, 6H), 1.59 |
| | methyl-1H-pyrazol-4-yl)-8- | (d, J = 6.8 Hz, 3H). LCMS |
| | azabicyclo[3.2.1]octan-8-yl)quinoxalin-5- | [M + 23]⁺ = 544.3. |
| | yl)ethyl)amino)benzoic acid | First eluting isomer leading to |
| | AND | 99B: ¹H NMR (400 MHz, |
| | 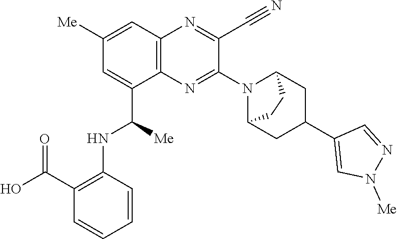 [SMILES: Cc1cc([C@@H](C)Nc2ccccc2C(=O)O)c2nc(N3[C@@H]4CC[C@H]3CC(c3cnn(C)c3)C4)c(C#N)nc2c1] | DMSO-d₆) δ = 12.69 (br s, 1H), 8.45 (br d, J = 6.8 Hz, 1H), 7.78 (dd, J = 1.6, 8.0 Hz, 1H), 7.64 (s, 1H), 7.61 (s, 1H), 7.59 (d, J = 1.6 Hz, 1H), 7.42 (s, 1H), 7.17-7.10 (m, 1H), 6.49 (t, J = 7.2 Hz, 1H), 6.40 (d, J = 8.4 Hz, 1H), 5.48 (m, 1H), 5.00 (br d, J = 2.4 Hz, 2H), 3.78 (s, 3H), 2.98-2.87 (m, 1H), 2.49-2.44 (m, 2H), 2.40 (s, 3H), 2.06-1.97 (m, 2H), 1.96-1.88 (m, 2H), 1.75- 1.66 (m, 2H), 1.60 (d, J = 6.8 Hz, 3H). LCMS [M + 1]⁺ = |
| | 2-(((1R)-1-(2-cyano-7-methyl-3-((1R,5S)-3-(1- | 522.3. |
| | methyl-1H-pyrazol-4-yl)-8- | |
| | azabicyclo[3.2.1]octan-8-yl)quinoxalin-5- | |
| | yl)ethyl)amino)benzoic acid | |
| 100 | 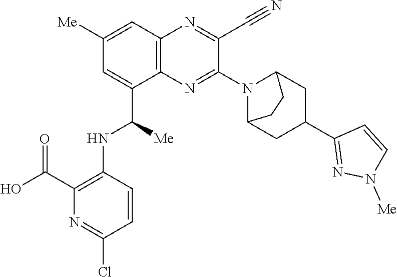 [SMILES: Cc1cc([C@@H](C)Nc2ccc(Cl)nc2C(=O)O)c2nc(N3C4CCC3CC(c3ccn(C)n3)C4)c(C#N)nc2c1] | |
| | 6-chloro-3-(((1R)-1-(2-cyano-7-methyl-3-(3-(1- | |
| | methyl-1H-pyrazol-3-yl)-8- | |
| | azabicyclo[3.2.1]octan-8-yl)quinoxalin-5- | |
| | yl)ethyl)amino)picolinic acid | |
| 101 | 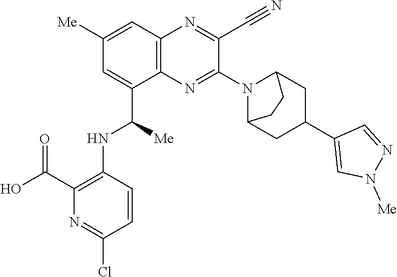 [SMILES: Cc1cc([C@@H](C)Nc2ccc(Cl)nc2C(=O)O)c2nc(N3C4CCC3CC(c3cnn(C)c3)C4)c(C#N)nc2c1] | |
| | 6-chloro-3-(((1R)-1-(2-cyano-7-methyl-3-(3-(1- | |
| | methyl-1H-pyrazol-4-yl)-8- | |
| | azabicyclo[3.2.1]octan-8-yl)quinoxalin-5- | |
| | yl)ethyl)amino)picolinic acid | |
| 102 | 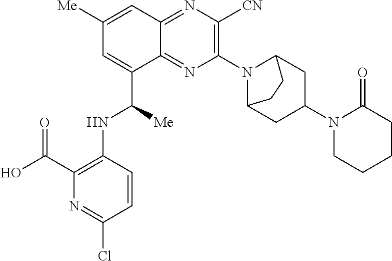 [SMILES: Cc1cc([C@@H](C)Nc2ccc(Cl)nc2C(=O)O)c2nc(N3C4CCC3CC(N3CCCCC3=O)C4)c(C#N)nc2c1] | |
| | 6-chloro-3-(((1R)-1-(2-cyano-7-methyl-3-(3-(2- | |
| | oxopiperidin-1-yl)-8-azabicyclo[3.2.1]octan-8- | |
| | yl)quinoxalin-5-yl)ethyl)amino)picolinic acid | |
| 103 | 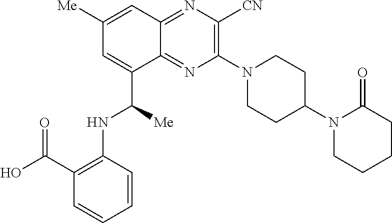 [SMILES: Cc1cc([C@@H](C)Nc2ccccc2C(=O)O)c2nc(N3CCC(N4CCCCC4=O)CC3)c(C#N)nc2c1] | |
| | (R)-2-((1-(2-cyano-7-methyl-3-(2-oxo-[1,4′- | |
| | bipiperidin]-1′-yl)quinoxalin-5- | |
| | yl)ethyl)amino)benzoic acid | |
| 104 | 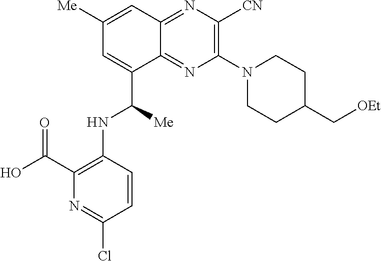 [SMILES: CCOCC1CCN(c2nc3c([C@@H](C)Nc4ccc(Cl)nc4C(=O)O)cc(C)cc3nc2C#N)CC1] | |
| | (R)-6-chloro-3-((1-(2-cyano-3-(4- | |
| | (ethoxymethyl)piperidin-1-yl)-7- | |
| | methylquinoxalin-5-yl)ethyl)amino)picolinic acid | |
| 105 | 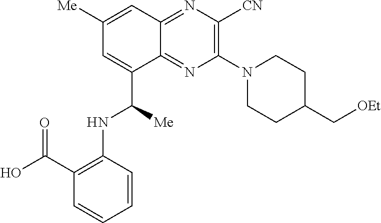 [SMILES: CCOCC1CCN(c2nc3c([C@@H](C)Nc4ccccc4C(=O)O)cc(C)cc3nc2C#N)CC1] | |
| | (R)-2-((1-(2-cyano-3-(4-(ethoxymethyl)piperidin- | |
| | 1-yl)-7-methylquinoxalin-5- | |
| | yl)ethyl)amino)benzoic acid | |
| 106 | 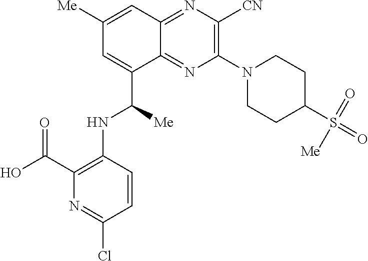 [SMILES: Cc1cc([C@@H](C)Nc2ccc(Cl)nc2C(=O)O)c2nc(N3CCC(S(C)(=O)=O)CC3)c(C#N)nc2c1] | ¹H NMR (400 MHz, CD₃OD) δ = 7.66 (d, J = 2.8 Hz, 2H), 7.21 (d, J = 8.8 Hz, 1H), 7.03 (d, J = 9.2 Hz, 1H), 5.57 (q, J = 6.8 Hz, 1H), 4.60-4.52 (m, 2H), 3.45 (tt, J = 3.6, 12.0 Hz, 1H), 3.29- 3.22 (m, 2H), 2.98 (s, 3H), 2.47 (s, 3H), 2.34-2.26 (m, 2H), 2.13-1.95 (m, 2H), 1.72 (d, J = 6.8 Hz, 3H). LCMS [M + 1]⁺ = 529.2. |
| | (R)-6-chloro-3-((1-(2-cyano-7-methyl-3-(4- | |
| | (methylsulfonyl)piperidin-1-yl)quinoxalin-5- | |
| | yl)ethyl)amino)picolinic acid | |
| 107 | 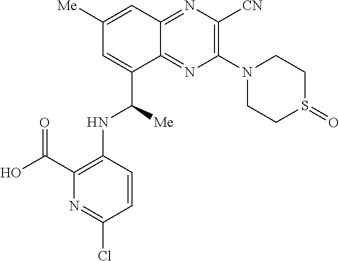 [SMILES: Cc1cc([C@@H](C)Nc2ccc(Cl)nc2C(=O)O)c2nc(N3CCS(=O)CC3)c(C#N)nc2c1] | ¹H NMR (400 MHz, CD₃OD) δ = 7.67 (d, J = 7.2 Hz, 2H), 7.20 (d, J = 8.8 Hz, 1H), 7.04 (d, J = 8.8 Hz, 1H), 5.55 (q, J = 6.8 Hz, 1H), 4.39-4.28 (m, 2H), 4.26- 4.15 (m, 2H), 3.29-3.21 (m, 2H), 3.11-3.03 (m, 2H), 2.47 (s, 3H), 1.74 (d, J = 6.8 Hz, 3H). LCMS [M + H]⁺ = 485.2. |
| | (R)-6-chloro-3-((1-(2-cyano-7-methyl-3-(1- | |
| | oxidothiomorpholino)quinoxalin-5- | |
| | yl)ethyl)amino)picolinic acid | |
| 108 | 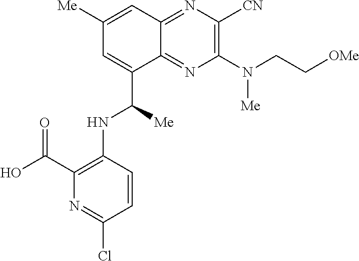 [SMILES: COCCN(C)c1nc2c([C@@H](C)Nc3ccc(Cl)nc3C(=O)O)cc(C)cc2nc1C#N] | ¹H NMR (400 MHz, CD₃OD) δ = 7.56 (s, 1H), 7.54 (s, 1H), 7.17 (d, J = 8.8 Hz, 1H), 6.96 (d, J = 8.8 Hz, 1H), 5.51 (q, J = 6.4 Hz, 1H), 4.13-3.98 (m, 2H), 3.74 (t, J = 5.2 Hz, 2H), 3.44 (s, 3H), 3.30 (br s, 3H), 2.41 (s, 3H), 1.68 (d, J = 6.8 Hz, 3H). LCMS [M + H]⁺ = 455.2. |
| | (R)-6-chloro-3-((1-(2-cyano-3-((2- | |
| | methoxyethyl)(methyl)amino)-7- | |
| | methylquinoxalin-5-yl)ethyl)amino)picolinic acid | |
| 109 | 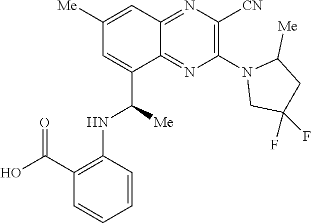 [SMILES: Cc1cc([C@@H](C)Nc2ccccc2C(=O)O)c2nc(N3CC(F)(F)CC3C)c(C#N)nc2c1] | |
| | 2-(((1R)-1-(2-cyano-3-(4,4-difluoro-2- | |
| | methylpyrrolidin-1-yl)-7-methylquinoxalin-5- | |
| | yl)ethyl)amino)benzoic acid | |
| 111A and 111B | 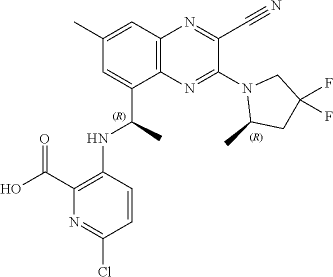 | Separated isomers by SFC before saponification: Final product from Saponification of first eluting methyl ester isomer (111A): ¹H NMR (400 MHz, CD₃OD) δ = 7.63 (d, J = 1.6 Hz, 1H), 7.57 (s, 1H), 7.18 (d, J = 8.8 Hz, 1H), 6.99 (d, J = 9.0 Hz, 1H), 5.50 (q, J = 6.8 Hz, 1H), 5.12-5.02 (m, 1H), 4.53-4.40 (m, 1H), 4.29 (q, J = 12.0 Hz, 1H), 2.77 (m, 1H), 2.43 (s, 3H), 2.34 (dq, J = 5.2, 13.6 Hz, 1H), 1.73 (d, J = 6.4 Hz, 3H), 1.48 (d, J = 6.8 Hz, 3H). LCMS [M + Na]⁺ = 509.1. |
| | 6-chloro-3-(((R)-1-(2-cyano-3-((R)-4,4-difluoro-2- | Final product from |
| | methylpyrrolidin-1-yl)-7-methylquinoxalin-5- | Saponification of second |
| | yl)ethyl)amino)picolinic acid | eluting methyl ester isomer |
| | and | (111B): ¹H NMR (400 MHz, |
| | 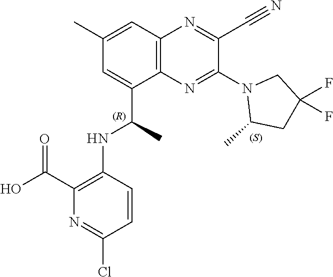 | CD₃OD) δ = 7.60 (s, 1H), 7.58 (s, 1H), 7.14 (d, J = 8.8 Hz, 1H), 6.89 (d, J = 8.8 Hz, 1H), 5.59 (q, J = 6.8 Hz, 1H), 5.01-4.92 (m, 1H), 4.45 (q, J = 12.0 Hz, 1H, 1H), 4.31 (q, J = 12.0 Hz, 1H), 2.87-2.66 (m, 1H), 2.41 (s, 3H), 2.35 (m,, 1H), 1.66 (d, J = 6.8 Hz, 3H), 1.49 (d, J = 6.8 Hz, 3H). LCMS [M + Na]⁺ = 509.1. |
| | 6-chloro-3-(((R)-1-(2-cyano-3-((S)-4,4-difluoro-2- | |
| | methylpyrrolidin-1-yl)-7-methylquinoxalin-5- | |
| | yl)ethyl)amino)picolinic acid | |
| 112A and 112B | 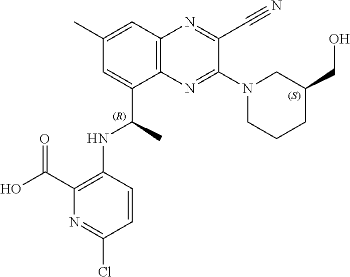 | Separated isomers by SFC before saponification: Final product from saponification of first eluting methyl ester isomer (112A): ¹H NMR (400 MHz, DMSO-d₆) δ = 12.98 (br s, 1H), 8.45 (d, J = 6.8 Hz, 1H), 7.64 (d, J = 0.8 Hz, 1H), 7.63 (d, J = 1.6 Hz, 1H), 7.29 (d, J = 8.8 Hz, 1H), 7.01 (d, J = 9.2 Hz, 1H), 5.49 (quin, J = 6.8 Hz, 1H), 4.58 (br s, 1H), 4.32 (br d, J = 12.8 Hz, 1H), 4.22 (br d, J = 13.6 Hz, 1H), 3.46-3.34 (m, 2H), 3.24-3.11 (m, 1H), 3.00 (dd, J = 10.0, 12.8 Hz, 1H), 2.44 (br s, 3H), 1.82 |
| | 6-chloro-3-(((R)-1-(2-cyano-3-((S)-3- | (br d, J = 8.8 Hz, 3H), 1.75- |
| | (hydroxymethyl)piperidin-1-yl)-7- | 1.66 (m, 1H), 1.64 (d, J = 6.4 |
| | methylquinoxalin-5-yl)ethyl)amino)picolinic acid | Hz, 3H), 1.34-1.22 (m, 1H). |
| | and | LCMS [M + Na]⁺ = 503.3. |
| | 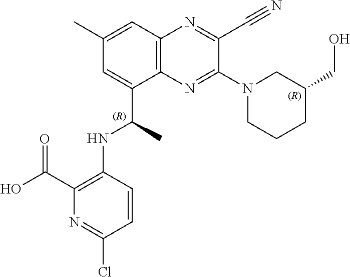 | Final product from Saponification of second eluting methyl ester isomer (112B): ¹H NMR (400 MHz, DMSO-d₆) δ = 12.98 (br s, 1H), 8.46 (d, J = 7.2 Hz, 1H), 7.64 (d, J = 0.8 Hz, 1H), 7.63 (d, J = 2.0 Hz, 1H), 7.29 (d, J = 9.2 Hz, 1H), 7.02 (d, J = 9.2 Hz, 1H), 5.48 (quin, J = 6.8 Hz, 1H), 4.98- 4.43 (m, 1H), 4.34-4.31 (m, 1H), 4.20 (br d, J = 13.6 Hz, 1H), 3.46-3.36 (m, 2H), 3.21 (m, 1H), 2.99 (dd, J = 10.0, 13.2 Hz, 1H), 2.42 (s, 3H), 1.94- 1.76 (m, 3H), 1.69 (m, 1H), 1.64 (d, J = 6.8 Hz, 3H), 1.44-1.15 |
| | 6-chloro-3-(((R)-1-(2-cyano-3-((R)-3- | (m, 1H). LCMS [M + Na]⁺ = |
| | (hydroxymethyl)piperidin-1-yl)-7- | 503.3. |
| | methylquinoxalin-5-yl)ethyl)amino)picolinic acid | |
| 113 | 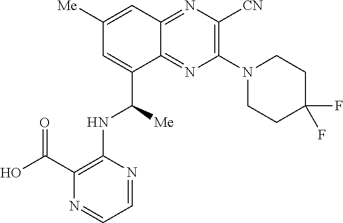 [SMILES: Cc1cc([C@@H](C)Nc2nccnc2C(=O)O)c2nc(N3CCC(F)(F)CC3)c(C#N)nc2c1] | ¹H NMR (400 MHz, CDCl₃) δ = 8.92-8.74 (m, 1H), 8.25 (br s, 1H), 7.72 (s, 1H), 7.66 (s, 1H), 7.60 (s, 1H), 6.02 (m, 1H), 4.01-3.81 (m, 4H), 2.50 (s, 3H), 2.32-2.21 (m, 4H), 1.76 (d, J = 6.8 Hz, 3H). LCMS [M + H]⁺: = 454.2. LCMS [M + Na]⁺ = 476.2. |
| | (R)-3-((1-(2-cyano-3-(4,4-difluoropiperidin-1-yl)- | |
| | 7-methylquinoxalin-5-yl)ethyl)amino)pyrazine-2- | |
| | carboxylic acid | |
| 114 | 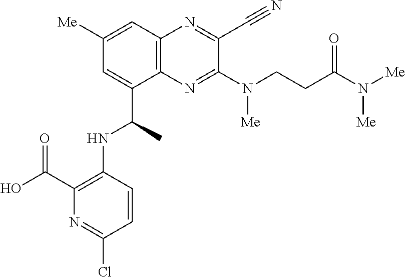 [SMILES: Cc1cc([C@@H](C)Nc2ccc(Cl)nc2C(=O)O)c2nc(N(C)CCC(=O)N(C)C)c(C#N)nc2c1] | ¹H NMR (400 MHz, CD₃OD) δ = 7.59-7.51 (m, 2H), 7.24- 7.13 (m, 1H), 6.99 (d, J = 9.2 Hz, 1H), 5.59-5.53 (m, 1H), 4.19-4.02 (m, 2H), 3.44 (s, 3H), 3.04 (s, 3H), 2.93-2.92 (m, 2H), 2.89 (s, 3H), 2.42 (d, J = 4.4 Hz, 3H), 1.68 (d, J = 6.8 Hz, 3H). LCMS [M + 1]⁺ = 496.3. |
| | (R)-6-chloro-3-((1-(2-cyano-3-((3- | |
| | (dimethylamino)-3-oxopropyl)(methyl)amino)-7- | |
| | methylquinoxalin-5-yl)ethyl)amino)picolinic acid | |
| 115 | 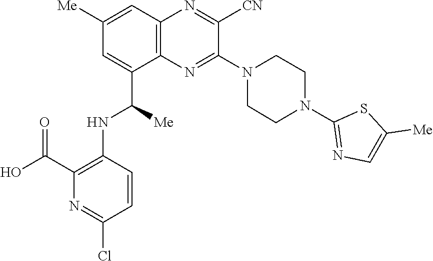 [SMILES: Cc1cc([C@@H](C)Nc2ccc(Cl)nc2C(=O)O)c2nc(N3CCN(c4ncc(C)s4)CC3)c(C#N)nc2c1] | ¹H NMR (400 MHz, CDCl₃) δ = 8.40 (br d, J = 6.8 Hz, 1H), 7.70 (s, 1H), 7.58 (d, J = 1.6 Hz, 1H), 7.14 (d, J = 8.8 Hz, 1H), 6.94 (s, 1H), 6.80 (d, J = 8.8 Hz, 1H), 5.48 (quin, J = 6.8 Hz, 1H), 3.93-3.92 (m, 4H), 3.90-3.79 (m, 4H), 2.49 (s, 3H), 2.35 (d, J = 0.8 Hz, 3H), 1.72 (d, J = 6.8 Hz, 3H). LCMS [M + 1]⁺ = 549.2. |
| | (R)-6-chloro-3-((1-(2-cyano-7-methyl-3-(4-(5- | |
| | methylthiazol-2-yl)piperazin-1-yl)quinoxalin-5- | |
| | yl)ethyl)amino)picolinic acid | |
| 116 | 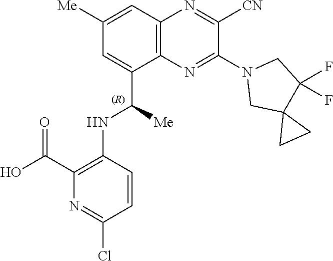 | ¹H NMR (400 MHz, CD₃OD) δ = 7.62 (s, 1H), 7.54 (s, 1H), 7.19 (d, J = 9.2 Hz, 1H), 7.05 (d, J = 9.2 Hz, 1H), 5.50 (q, J = 6.8 Hz, 1H), 4.46 (dt, J = 6.0, 12.0 Hz, 2H), 4.23-4.08 (m, 2H), 2.44 (s, 3H), 1.74 (d, J = 6.4 Hz, 3H), 1.21-1.13 (m, 2H), 0.99 (br s, 2H). |
| | (R)-6-chloro-3-((1-(2-cyano-3-(7,7-difluoro-5- | |
| | azaspiro[2.4]heptan-5-yl)-7-methylquinoxalin-5- | |
| | yl)ethyl)amino)picolinic acid | |
| 117 | 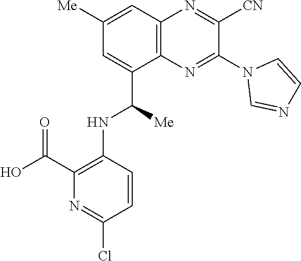 [SMILES: Cc1cc([C@@H](C)Nc2ccc(Cl)nc2C(=O)O)c2nc(-n3ccnc3)c(C#N)nc2c1] | ¹H NMR (400 MHz, DMSO-d₆) δ = 8.88 (s, 1H), 8.55 (br d, J = 7.2 Hz, 1H), 8.17 (s, 1H), 8.00 (s, 1H), 7.97 (s, 1H), 7.44 (s, 1H), 7.27 (d, J = 8.8 Hz, 1H), 7.11 (d, J = 9.2 Hz, 1H), 5.70- 5.60 (m, 1H), 2.57 (s, 3H), 1.69 (d, J = 6.8 Hz, 3H). LCMS [M + 1]⁺ = 434.2. |
| | (R)-6-chloro-3-((1-(2-cyano-3-(1H-imidazol-1-yl)- | |
| | 7-methylquinoxalin-5-yl)ethyl)amino)picolinic | |
| | acid | |
| 118 | 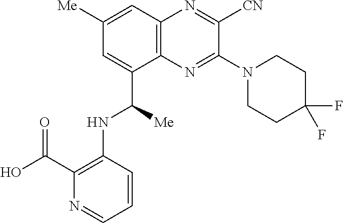 [SMILES: Cc1cc([C@@H](C)Nc2cccnc2C(=O)O)c2nc(N3CCC(F)(F)CC3)c(C#N)nc2c1] | ¹H NMR (400 MHz, CDCl₃) δ = 8.54-8.27 (m, 1H), 7.91-7.75 (m, 1H), 7.67 (s, 1H), 7.61 (d, J = 1.6 Hz, 1H), 7.21-7.09 (m, 1H), 6.76 (d, J = 8.8 Hz, 1H), 5.52 (quin, J = 6.8 Hz, 1H), 3.98-3.83 (m, 4H), 2.47 (s, 3H), 2.34-2.19 (m, 4H), 1.71 (d, J = 6.8 Hz, 3H). LCMS [M + 1]⁺ = 453.3. |
| | (R)-3-((1-(2-cyano-3-(4,4-difluoropiperidin-1-yl)- | |
| | 7-methylquinoxalin-5-yl)ethyl)amino)picolinic | |
| | acid | |
| 119 | 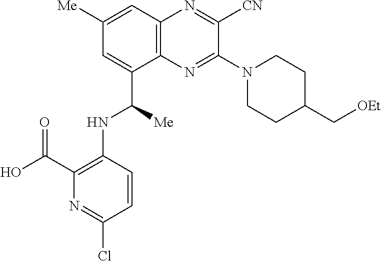 [SMILES: CCOCC1CCN(c2nc3c([C@@H](C)Nc4ccc(Cl)nc4C(=O)O)cc(C)cc3nc2C#N)CC1] | ¹H NMR (400 MHz, CD₃OD) δ = 7.62-7.61 (m, 2H), 7.20 (d, J = 9.2 Hz, 1H), 7.02 (d, J = 9.2 Hz, 1H), 5.55 (q, J = 6.8 Hz, 1H), 4.44-4.40 (m, 2H), 3.52 (q, J = 7.2 Hz, 2H), 3.38 (d, J = 6.0 Hz, 2H), 3.18-3.14 (m, 2H), 2.45 (s, 3H), 1.95-1.92 (m, 3H), 1.72 (d, J = 6.4 Hz, 3H), 1.53-1.49 (m, 2H), 1.20 (t, J = 7.2 Hz, 3H). LCMS [M + H]⁺ = 509.2. |
| | (R)-6-chloro-3-((1-(2-cyano-3-(4- | |
| | (ethoxymethyl)piperidin-1-yl)-7- | |
| | methylquinoxalin-5-yl)ethyl)amino)picolinic acid | |
| 120 | 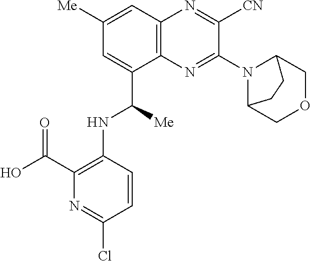 [SMILES: Cc1cc([C@@H](C)Nc2ccc(Cl)nc2C(=O)O)c2nc(N3C4CCC3COC4)c(C#N)nc2c1] | ¹H NMR (400 MHz, CDCl₃) δ = 8.34 (br d, J = 5.6 Hz, 1H), 7.64 (s, 1H), 7.53 (s, 1H), 7.10 (d, J = 8.8 Hz, 1H), 6.73 (d, J = 8.8 Hz, 1H), 5.49-5.44 (m, 1H), 4.88 (br d, J = 5.6 Hz, 2H), 4.03-3.98 (m, 2H), 3.80- 3.76 (m, 2H), 2.46 (s, 3H), 2.24-2.17 (m, 2H), 2.12-2.02 (m, 2H), 1.69 (d, J = 6.4 Hz, 3H). LCMS [M + Na]⁺ = 501.2. |
| | 3-(((1R)-1-(3-(3-oxa-8-azabicyclo[3.2.1]octan-8- | |
| | yl)-2-cyano-7-methylquinoxalin-5- | |
| | yl)ethyl)amino)-6-chloropicolinic acid | |
| 121 | 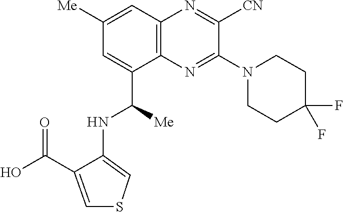 [SMILES: Cc1cc([C@@H](C)Nc2cscc2C(=O)O)c2nc(N3CCC(F)(F)CC3)c(C#N)nc2c1] | ¹H NMR (400 MHz, CDCl₃) δ = 8.09 (d, J = 3.2 Hz, 1H), 7.69 (d, J = 1.6 Hz, 1H), 7.66 (s, 1H), 5.46 (d, J = 3.6 Hz, 1H), 5.30 (q, J = 6.8 Hz, 1H), 3.92- 3.83 (m, 4H), 2.50 (s, 3H), 2.31-2.22 (m, 4H), 1.66 (d, J = 6.8 Hz, 3H). LCMS [M + 1]⁺: 458.2. |
| | (R)-4-((1-(2-cyano-3-(4,4-difluoropiperidin-1-yl)- | |
| | 7-methylquinoxalin-5-yl)ethyl)amino)thiophene- | |
| | 3-carboxylic acid | |
| 122 | 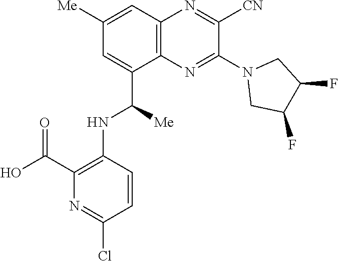 [SMILES: Cc1cc([C@@H](C)Nc2ccc(Cl)nc2C(=O)O)c2nc(N3C[C@@H](F)[C@@H](F)C3)c(C#N)nc2c1] | ¹H NMR (400 MHz, CD₃OD) δ = 7.60 (d, J = 1.6 Hz, 1H), 7.56 (s, 1H), 7.18 (d, J = 9.2 Hz, 1H), 7.02 (d, J = 9.2 Hz, 1H), 5.59- 5.39 (m, 2H), 5.37-5.24 (m, 1H), 4.47-4.24 (m, 2H), 4.23- 4.03 (m, 2H), 2.42 (s, 3H), 1.73 (d, J = 6.4 Hz, 3H). LCMS [M + Na]⁺ = 495.1. |
| | 6-chloro-3-(((R)-1-(2-cyano-3-((3S,4R)-3,4- | |
| | difluoropyrrolidin-1-yl)-7-methylquinoxalin-5- | |
| | yl)ethyl)amino)picolinic acid | |
| 123 | 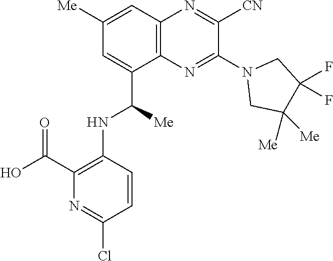 [SMILES: Cc1cc([C@@H](C)Nc2ccc(Cl)nc2C(=O)O)c2nc(N3CC(C)(C)C(F)(F)C3)c(C#N)nc2c1] | ¹H NMR (400 MHz, CD₃OD) δ = 7.61 (d, J = 2.0 Hz, 1H), 7.60-7.57 (m, 1H), 7.20 (d, J = 9.2 Hz, 1H), 7.06 (d, J = 9.2 Hz, 1H), 5.48 (q, J = 6.4 Hz, 1H), 4.45-4.26 (m, 2H), 4.00-3.87 (m, 2H), 2.44 (s, 3H), 1.74 (d, J = 6.8 Hz, 3H), 1.28 (s, 6H). LCMS [M + H]⁺ = 501.1. |
| | (R)-6-chloro-3-((1-(2-cyano-3-(3,3-difluoro-4,4- | |
| | dimethylpyrrolidin-1-yl)-7-methylquinoxalin-5- | |
| | yl)ethyl)amino)picolinic acid | |
| 124 | 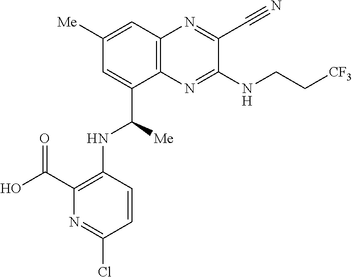 [SMILES: Cc1cc([C@@H](C)Nc2ccc(Cl)nc2C(=O)O)c2nc(NCCC(F)(F)F)c(C#N)nc2c1] | ¹H NMR (400 MHz, CD₃OD) δ = 7.63 (s, 1H), 7.57 (s, 1H), 7.22 (d, J = 9.2 Hz, 1H), 6.99 (d, J = 9.2 Hz, 1H), 5.61-5.52 (m, 1H), 4.01-3.84 (m, 2H), 2.73-2.60 (m, 2H), 2.43 (s, 3H), 1.70 (d, J = 6.8 Hz, 3H). |
| | (R)-6-chloro-3-((1-(2-cyano-7-methyl-3-((3,3,3- | |
| | trifluoropropyl)amino)quinoxalin-5- | |
| | yl)ethyl)amino)picolinic acid | |
| 125 | 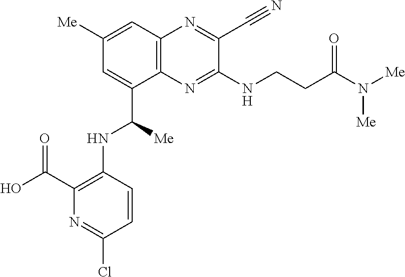 [SMILES: Cc1cc([C@@H](C)Nc2ccc(Cl)nc2C(=O)O)c2nc(NCCC(=O)N(C)C)c(C#N)nc2c1] | ¹H NMR (400 MHz, CD₃OD) δ = 7.56 (d, J = 1.2 Hz, 1H), 7.51 (s, 1H), 7.19 (d, J = 8.8 Hz, 1H), 7.04 (d, J = 9.2 Hz, 1H), 5.59 (q, J = 6.4 Hz, 1H), 3.91 (dt, J = 2.4, 6.8 Hz, 2H), 3.03 (s, 3H), 2.94 (s, 3H), 2.82 (t, J = 6.8 Hz, 2H), 2.40 (s, 3H), 1.69 (d, J = 6.8 Hz, 3H). LCMS [M + 1]⁺ = 482.3. |
| | (R)-6-chloro-3-((1-(2-cyano-3-((3- | |
| | (dimethylamino)-3-oxopropyl)amino)-7- | |
| | methylquinoxalin-5-yl)ethyl)amino)picolinic acid | |
| 126 | 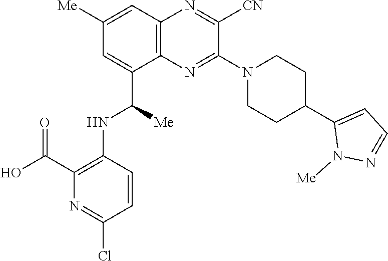 [SMILES: Cc1cc([C@@H](C)Nc2ccc(Cl)nc2C(=O)O)c2nc(N3CCC(c4ccnn4C)CC3)c(C#N)nc2c1] | ¹H NMR (400 MHz, CD₃OD) δ = 7.65 (d, J = 1.6 Hz, 1H), 7.62 (s, 1H), 7.37 (d, J = 2.0 Hz, 1H), 7.19 (d, J = 8.8 Hz, 1H), 7.05 (d, J = 9.2 Hz, 1H), 6.14 (d, J = 2.0 Hz, 1H), 5.55 (q, J = 6.8 Hz, 1H), 4.58-4.42 (m, 2H), 3.88 (s, 3H), 3.40-3.32 (m, 1H), 3.32-3.26 (m, 1H), 3.11 (tt, J = 3.6, 11.8 Hz, 1H), 2.45 (s, 3H), 2.12 (br d, J = 12.0 Hz, 2H), 1.90 (dq, J = 4.0, 12.4 Hz, 2H), 1.73 (d, J = 6.8 Hz, 3H). LCMS [M + 1]⁺ = 531.3. |
| | (R)-6-chloro-3-((1-(2-cyano-7-methyl-3-(4-(1- | |
| | methyl-1H-pyrazol-5-yl)piperidin-1-yl)quinoxalin- | |
| | 5-yl)ethyl)amino)picolinic acid | |
| 127 | 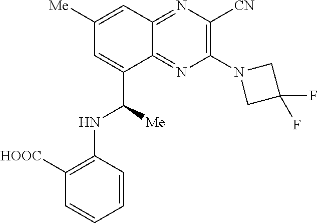 [SMILES: Cc1cc([C@@H](C)Nc2ccccc2C(=O)O)c2nc(N3CC(F)(F)C3)c(C#N)nc2c1] | ¹H NMR (400 MHz, CD₃OD) δ = 7.88 (dd, J = 8 Hz, 1H), 7.65 (s, 1H), 7.59 (s, 1H), 7.15-7.07 (m, 1H), 6.50-6.46 (m, 1H), 6.42-6.44 (m, 1H), 5.54 (q, J = 6.8 Hz, 1H), 4.87-4.76(m, 4H), 2.43 (s, 3H), 1.69 (d, J = 6.4 Hz, 3H). LCMS [M + H]⁺: 424.3. |
| | (R)-2-((1-(2-cyano-3-(3,3-difluoroazetidin-1-yl)- | |
| | 7-methylquinoxalin-5-yl)ethyl)amino)benzoic | |
| | acid | |
| 128 | 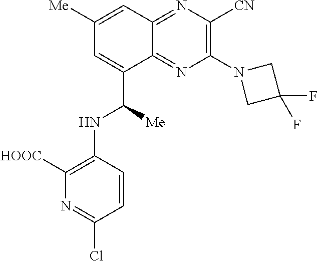 [SMILES: Cc1cc([C@@H](C)Nc2ccc(Cl)nc2C(=O)O)c2nc(N3CC(F)(F)C3)c(C#N)nc2c1] | ¹H NMR (400 MHz, CD₃OD) δ = 7.65 (d, J = 1.6 Hz, 1H), 7.59 (s, 1H), 7.18 (d, J = 8.8 Hz, 1H), 7.04 (d, J = 8.8 Hz, 1H), 5.49 (q, J = 6.8 Hz, 1H), 4.84-4.76 (m, 4H), 2.44 (s, 3H), 1.72 (d, J = 6.4 Hz, 3H). |
| | (R)-6-chloro-3-((1-(2-cyano-3-(3,3- | |
| | difluoroazetidin-1-yl)-7-methylquinoxalin-5- | |
| | yl)ethyl)amino)picolinic acid | |
| 129 | 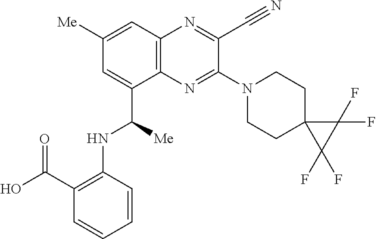 [SMILES: Cc1cc([C@@H](C)Nc2ccccc2C(=O)O)c2nc(N3CCC4(CC3)C(F)(F)C4(F)F)c(C#N)nc2c1] | ¹H NMR (400 MHz, CD₃OD) δ = 7.88 (dd, J = 1.6, 8.0 Hz, 1H), 7.66 (s, 1H), 7.64 (s, 1H), 7.11 (ddd, J = 1.6, 7.2, 8.4 Hz, 1H), 6.54-6.46 (m, 1H), 6.40 (d, J = 8.4 Hz, 1H), 5.59 (q, J = 6.8 Hz, 1H), 3.87-3.79 (m, 4H), 2.45 (s, 3H), 2.09 (br s, 4H), 1.68 (d, J = 6.8 Hz, 3H). LCMS [M + H]⁺ = 514.2. |
| | (R)-2-((1-(2-cyano-7-methyl-3-(1,1,2,2- | |
| | tetrafluoro-6-azaspiro[2.5]octan-6-yl)quinoxalin- | |
| | 5-yl)ethyl)amino)benzoic acid | |
| 130 | 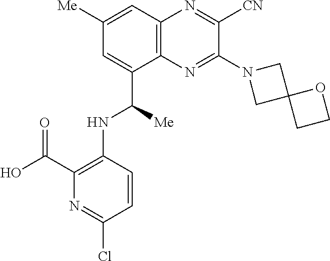 [SMILES: Cc1cc([C@@H](C)Nc2ccc(Cl)nc2C(=O)O)c2nc(N3CC4(CCO4)C3)c(C#N)nc2c1] | ¹H NMR (400 MHz, CD₃OD) δ = 7.60 (d, J = 1.6 Hz, 1H), 7.57 (s, 1H), 7.21 (d, J = 8.8 Hz, 1H), 7.04 (d, J = 9.2 Hz, 1H), 5.49 (q, J = 6.8 Hz, 1H), 4.74 (dd, J = 1.2, 10.4 Hz, 1H), 4.71-4.66 (m, 1H), 4.63-4.54 (m, 4H), 3.01 (t, J = 7.6 Hz, 2H), 2.43 (s, 3H), 1.72 (d, J = 6.8 Hz, 3H). LCMS [M + H]⁺: 465.1. |
| | (R)-6-chloro-3-((1-(2-cyano-7-methyl-3-(1-oxa-6- | |
| | azaspiro[3.3]heptan-6-yl)quinoxalin-5- | |
| | yl)ethyl)amino)picolinic acid | |
| 131 | 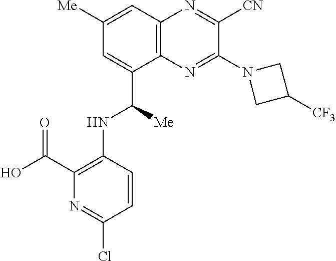 [SMILES: Cc1cc([C@@H](C)Nc2ccc(Cl)nc2C(=O)O)c2nc(N3CC(C(F)(F)F)C3)c(C#N)nc2c1] | ¹H NMR (400 MHz, CD₃OD) δ = 7.59 (d, J = 1.6 Hz, 1H), 7.47 (s, 1H), 7.15 (d, J = 8.8 Hz, 1H), 7.03 (d, J = 8.8 Hz, 1H), 5.47 (q, J = 6.8 Hz, 1H), 4.76-4.66 (m, 2H), 4.55-4.44 (m, 2H), 3.68 (m, 1H), 2.40 (s, 3H), 1.70 (d, J = 6.8 Hz, 3H). LCMS [M + Na]⁺ = 513.1. |
| | (R)-6-chloro-3-((1-(2-cyano-7-methyl-3-(3- | |
| | (trifluoromethyl)azetidin-1-yl)quinoxalin-5- | |
| | yl)ethyl)amino)picolinic acid | |
| 132 | 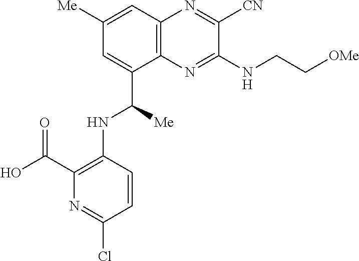 [SMILES: COCCNc1nc2c([C@@H](C)Nc3ccc(Cl)nc3C(=O)O)cc(C)cc2nc1C#N] | ¹H NMR (400 MHz, CD₃OD) δ = 7.56 (d, J = 1.6 Hz, 1H), 7.54 (s, 1H), 7.20 (d, J = 9.2 Hz, 1H), 7.00 (d, J = 9.2 Hz, 1H), 5.55 (q, J = 6.8 Hz, 1H), 3.90-3.75 (m, 2H), 3.72-3.63 (m, 2H), 3.41 (s, 3H), 2.41 (s, 3H), 1.70 (d, J = 6.8 Hz, 3H). LCMS [M + Na]⁺ = 463.1. |
| | (R)-6-chloro-3-((1-(2-cyano-3-((2- | |
| | methoxyethyl)amino)-7-methylquinoxalin-5- | |
| | yl)ethyl)amino)picolinic acid | |
| 133 | 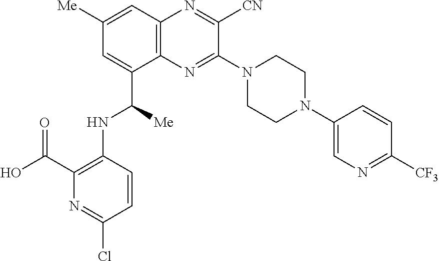 [SMILES: Cc1cc([C@@H](C)Nc2ccc(Cl)nc2C(=O)O)c2nc(N3CCN(c4ccc(C(F)(F)F)nc4)CC3)c(C#N)nc2c1] | ¹H NMR (400 MHz, CD₃OD) δ = 8.43 (d, J = 2.8 Hz, 1H), 7.68 (s, 2H), 7.66 (d, J = 8.8 Hz, 1H), 7.53 (dd, J = 2.8, 8.8 Hz, 1H), 7.21 (d, J = 9.2 Hz, 1H), 7.07 (d, J = 9.2 Hz, 1H), 5.56 (q, J = 6.4 Hz, 1H), 4.09-3.91 (m, 4H), 3.67 (t, J = 5.2 Hz, 4H), 2.48 (s, 3H), 1.76 (d, J = 6.8 Hz, 3H). LCMS [M + Na]⁺ = 597.3. |
| | (R)-6-chloro-3-((1-(2-cyano-7-methyl-3-(4-(6- | |
| | (trifluoromethyl)pyridin-3-yl)piperazin-1- | |
| | yl)quinoxalin-5-yl)ethyl)amino)picolinic acid | |
| 134 | 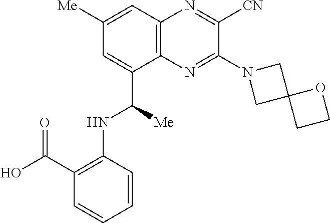 [SMILES: Cc1cc([C@@H](C)Nc2ccccc2C(=O)O)c2nc(N3CC4(CCO4)C3)c(C#N)nc2c1] | ¹H NMR (400 MHz, CDCl₃) δ = 8.52-8.10 (m, 1H), 7.99 (dd, J = 1.6, 8.0 Hz, 1H), 7.57 (s, 1H), 7.55 (s, 1H), 7.19 (ddd, J = 1.6, 7.2, 8.8 Hz, 1H), 6.62-6.53 (m, 1H), 6.38 (d, J = 8.4 Hz, 1H), 5.50 (q, J = 6.4 Hz, 1H), 4.69 (m, J = 4.0 Hz, 2H), 4.68-4.55 (m, 4H), 3.08-2.93 (m, 2H), 2.43 (s, 3H), 1.67 (d, J = 6.8 Hz, 3H). LCMS [M + Na]⁺ = 452.3. |
| | (R)-2-((1-(2-cyano-7-methyl-3-(1-oxa-6- | |
| | azaspiro[3.3]heptan-6-yl)quinoxalin-5- | |
| | yl)ethyl)amino)benzoic acid | |
| 135 | 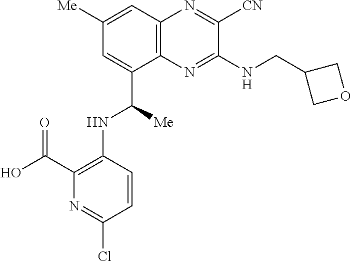 [SMILES: Cc1cc([C@@H](C)Nc2ccc(Cl)nc2C(=O)O)c2nc(NCC3COC3)c(C#N)nc2c1] | ¹H NMR (400 MHz, CD₃OD) δ = 7.57 (d, J = 2.0 Hz, 1H), 7.55 (s, 1H), 7.21 (d, J = 9.2 Hz, 1H), 6.99 (d, J = 9.2 Hz, 1H), 5.54 (q, J = 6.6 Hz, 1H), 4.85-4.83 (m, 2H),4.57 (t, J = 5.6 Hz, 2H), 4.02-3.86 (m, 2H), 3.52-3.41 (m, 1H), 2.42 (s, 3H), 1.70 (d, J = 6.8 Hz, 3H). LCMS [M + H]⁺ = 453.2. |
| | (R)-6-chloro-3-((1-(2-cyano-7-methyl-3-((oxetan- | |
| | 3-ylmethyl)amino)quinoxalin-5- | |
| | yl)ethyl)amino)picolinic acid | |
| 136 | 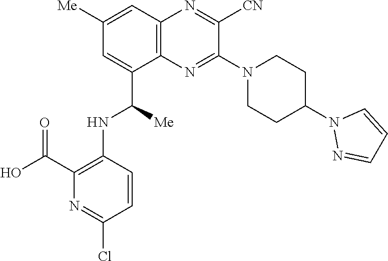 [SMILES: Cc1cc([C@@H](C)Nc2ccc(Cl)nc2C(=O)O)c2nc(N3CCC(n4cccn4)CC3)c(C#N)nc2c1] | ¹H NMR (400 MHz, CD₃OD) δ = 7.74 (d, J = 2.4 Hz, 1H), 7.68- 7.62 (m, 2H), 7.52 (d, J = 2.0 Hz, 1H), 7.21 (d, J = 8.8 Hz, 1H), 7.05 (d, J = 8.8 Hz, 1H), 6.31 (t, J = 2.0 Hz, 1H), 5.57 (q, J = 6.8 Hz, 1H), 4.61-4.51 (m, 3H), 3.43-3.33 (m, 2H), 2.46 (s, 3H), 2.36-2.22 (m, 4H), 1.73 (d, J = 6.4 Hz, 3H). LCMS [M + H]⁺ = 517.3. |
| | (R)-3-((1-(3-(4-(1H-pyrazol-1-yl)piperidin-1-yl)-2- | |
| | cyano-7-methylquinoxalin-5-yl)ethyl)amino)-6- | |
| | chloropicolinic acid | |
| 137 | 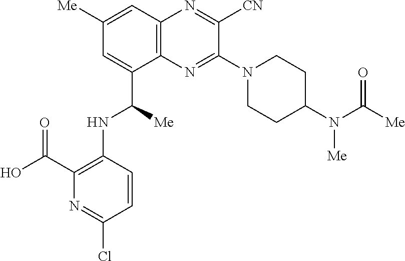 [SMILES: CC(=O)N(C)C1CCN(c2nc3c([C@@H](C)Nc4ccc(Cl)nc4C(=O)O)cc(C)cc3nc2C#N)CC1] | ¹H NMR (400 MHz, DMSO-d₆) δ = 13.35-11.41 (m, 1H), 8.39 (br d, J = 7.2 Hz, 1H), 7.67 (br d, J = 5.2 Hz, 2H), 7.28 (d, J = 8.8 Hz, 1H), 7.11 (br d, J = 9.2 Hz, 1H), 5.56-5.43 (m, 1H), 4.67-4.54 (m, 1H), 4.47 (br t, J = 11.2 Hz, 2H), 3.25 (br s, 2H), 2.86-2.67 (m, 3H), 2.50 (br s, 3H), 2.45 (s, 3H), 2.13-2.02 (m, 2H), 1.99-1.85 (m, 2H), 1.68 (br d, J = 6.4 Hz, 3H). LCMS [M + H]⁺ = 522.2. |
| | (R)-6-chloro-3-((1-(2-cyano-7-methyl-3-(4-(N- | |
| | methylacetamido)piperidin-1-yl)quinoxalin-5- | |
| | yl)ethyl)amino)picolinic acid | |
| 138 | 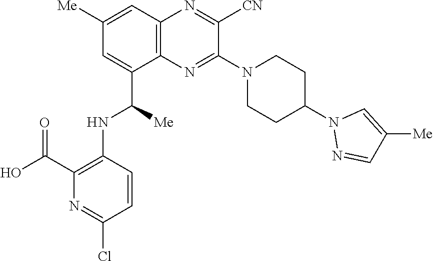 [SMILES: Cc1cc([C@@H](C)Nc2ccc(Cl)nc2C(=O)O)c2nc(N3CCC(n4cc(C)cn4)CC3)c(C#N)nc2c1] | ¹H NMR (400 MHz, CD₃OD) δ = 7.68 (s, 1H), 7.66 (s, 1H), 7.52 (s, 1H), 7.33 (s, 1H), 7.22 (d, J = 8.8 Hz, 1H), 7.07 (d, J = 9.2 Hz, 1H), 5.59 (q, J = 6.8 Hz, 1H), 4.61-4.51 (m, 2H), 4.50- 4.42 (m, 1H), 3.45-3.35 (m, 2H), 2.48 (s, 3H), 2.33-2.19 (m, 4H), 2.10 (s, 3H), 1.75 (d, J = 6.8 Hz, 3H). LCMS [M + H]⁺ = 531.2. |
| | (R)-6-chloro-3-((1-(2-cyano-7-methyl-3-(4-(4- | |
| | methyl-1H-pyrazol-1-yl)piperidin-1-yl)quinoxalin- | |
| | 5-yl)ethyl)amino)picolinic acid | |
| 139 | 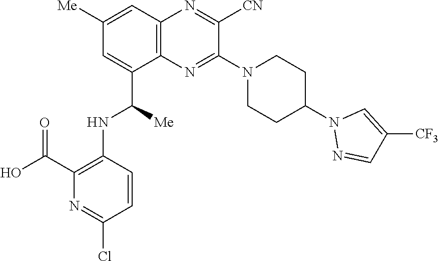 [SMILES: Cc1cc([C@@H](C)Nc2ccc(Cl)nc2C(=O)O)c2nc(N3CCC(n4cc(C(F)(F)F)cn4)CC3)c(C#N)nc2c1] | ¹H NMR (400 MHz, CD₃OD) δ = 8.22 (s, 1H), 7.78 (s, 1H), 7.69-7.60 (m, 2H), 7.21 (d, J = 8.8 Hz, 1H), 7.05 (d, J = 8.8 Hz, 1H), 5.57 (d, J = 6.4 Hz, 1H), 4.66-4.48 (m, 3H), 3.45-3.33 (m, 2H), 2.46 (s, 3H), 2.41- 2.25 (m, 4H), 1.73 (d, J = 6.8 Hz, 3H). LCMS [M + H]⁺ = 585.2. |
| | (R)-6-chloro-3-((1-(2-cyano-7-methyl-3-(4-(4- | |
| | (trifluoromethyl)-1H-pyrazol-1-yl)piperidin-1- | |
| | yl)quinoxalin-5-yl)ethyl)amino)picolinic acid | |
| 140 | 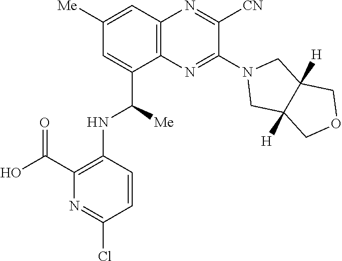 [SMILES: Cc1cc([C@@H](C)Nc2ccc(Cl)nc2C(=O)O)c2nc(N3C[C@H]4COC[C@H]4C3)c(C#N)nc2c1] | ¹H NMR (400 MHz, CD₃OD) δ = 7.59 (d, J = 2.0 Hz, 1H), 7.55 (s, 1H), 7.13 (d, J = 8.8 Hz, 1H), 6.94 (d, J = 9.2 Hz, 1H), 5.49 (q, J = 6.8 Hz, 1H), 4.21-4.10 (m, 2H), 4.05-3.86 (m, 4H), 3.80 (dt, J = 2.8, 8.8 Hz, 2H), 3.24-3.13 (m, 2H), 2.42 (s, 3H), 1.71 (d, J = 6.8 Hz, 3H). LCMS [M + 23]⁺ = 501.2. |
| | 6-chloro-3-(((R)-1-(2-cyano-7-methyl-3-cis- | |
| | tetrahydro-1H-furo[3,4-c]pyrrol-5(3H)- | |
| | yl)quinoxalin-5-yl)ethyl)amino)picolinic acid | |
| 141 | 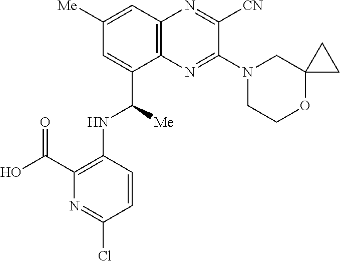 [SMILES: Cc1cc([C@@H](C)Nc2ccc(Cl)nc2C(=O)O)c2nc(N3CCOC4(CC4)C3)c(C#N)nc2c1] | ¹H NMR (400 MHz, CD₃OD) δ = 7.70-7.63 (m, 2H), 7.20 (d, J = 9.2 Hz, 1H), 7.03 (d, J = 8.8 Hz, 1H), 5.52 (q, J = 6.8 Hz, 1H), 4.02-3.96 (m, 2H), 3.89- 3.80 (m, 2H), 3.80-3.72 (m, 2H), 2.47 (s, 3H), 1.73 (d, J = 6.8 Hz, 3H), 0.92-0.77 (m, 4H). LCMS [M + 23]⁺ = 501.2. |
| | (R)-6-chloro-3-((1-(2-cyano-7-methyl-3-(4-oxa-7- | |
| | azaspiro[2.5]octan-7-yl)quinoxalin-5- | |
| | yl)ethyl)amino)picolinic acid | |
| 142 | 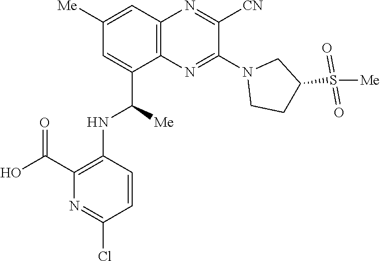 [SMILES: Cc1cc([C@@H](C)Nc2ccc(Cl)nc2C(=O)O)c2nc(N3CC[C@@H](S(C)(=O)=O)C3)c(C#N)nc2c1] | ¹H NMR (400 MHz, methanol-d₄) δ = 7.63 (s, 1H), 7.59 (s, 1H), 7.16 (br d, J = 8.8 Hz, 1H), 7.02 (br d, J = 8.8 Hz, 1H), 5.54 (q, J = 6.8 Hz, 1H), 4.44-4.40 (m, 1H), 4.34- 4.21 (m, 2H), 4.16-4.08 (m, 2H), 3.13 (s, 3H), 2.68-2.56 (m, 2H), 2.45 (s, 3H), 1.73 (d, J = 6.8 Hz, 3H), 1.32 (s, 1H). LCMS [M + 23]⁺ = 537.3. |
| | 6-chloro-3-(((R)-1-(2-cyano-7-methyl-3-((R)-3- | |
| | (methylsulfonyl)pyrrolidin-1-yl)quinoxalin-5- | |
| | yl)ethyl)amino)picolinic acid | |
| 143 | 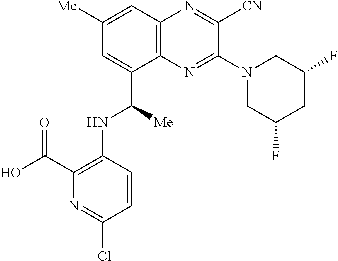 [SMILES: Cc1cc([C@@H](C)Nc2ccc(Cl)nc2C(=O)O)c2nc(N3C[C@H](F)C[C@H](F)C3)c(C#N)nc2c1] | ¹H NMR (400 MHz, methanol-d₄) δ = 7.69 (d, J = 2.0 Hz, 1H), 7.65 (d, J = 1.2 Hz, 1H), 7.10 (d, J = 8.8 Hz, 1H), 6.94 (d, J = 8.8 Hz, 1H), 5.60 (q, J = 6.8 Hz, 1H), 5.05-5.01 (m, 2H), 4.38-4.31 (m, 2H), 3.98- 3.89 (m, 2H), 2.47 (s, 3H), 2.45- 2.28 (m, 2H), 1.72 (d, J = 6.4 Hz, 3H). LCMS [M + 23]⁺ = 509.1. |
| | 6-chloro-3-(((R)-1-(2-cyano-3-((3S,5R)-3,5- | |
| | difluoropiperidin-1-yl)-7-methylquinoxalin-5- | |
| | yl)ethyl)amino)picolinic acid | |
| 144 | 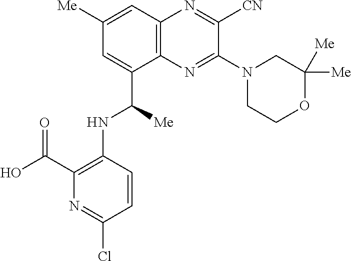 [SMILES: Cc1cc([C@@H](C)Nc2ccc(Cl)nc2C(=O)O)c2nc(N3CCOC(C)(C)C3)c(C#N)nc2c1] | ¹H NMR (400 MHz, methanol- d₄) δ = 7.70-7.61 (m, 2H), 7.20 (d, J = 8.8 Hz, 1H), 7.04 (d, J = 9.2 Hz, 1H), 5.53 (q, J = 6.8 Hz, 1H), 3.97 (t, J = 4.8 Hz, 2H), 3.80-3.66 (m, 2H), 3.66-3.56 (m, 2H), 2.47 (s, 3H), 1.74 (d, J = 6.8 Hz, 3H), 1.39 (s, 6H). LCMS [M + 23]⁺ = 503.2. |
| | (R)-6-chloro-3-((1-(2-cyano-3-(2,2- | |
| | dimethylmorpholino)-7-methylquinoxalin-5- | |
| | yl)ethyl)amino)picolinic acid | |
| 145 | 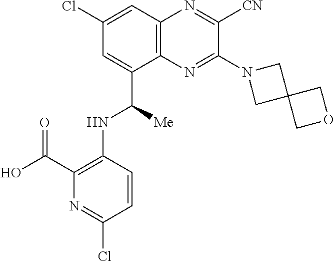 [SMILES: C[C@@H](Nc1ccc(Cl)nc1C(=O)O)c1cc(Cl)cc2nc(C#N)c(N3CC4(COC4)C3)nc12] | ¹H NMR (400 MHz, CDCl₃) δ = 8.44 (br d, J = 6.4 Hz, 1H), 7.82 (d, J = 2.0 Hz, 1H), 7.60 (d, J = 2.4 Hz, 1H), 7.16 (d, J = 8.8 Hz, 1H), 6.79 (d, J = 8.8 Hz, 1H), 5.37 (quin, J = 6.8 Hz, 1H), 4.99- 4.90 (m, 4H), 4.79-4.72 (m, 2H), 4.70-4.63 (m, 2H), 1.74 (d, J = 6.4 Hz, 3H). LCMS [M + 1]⁺ = 485.1. |
| | (R)-6-chloro-3-((1-(2-cyano-7-methyl-3-(2-oxa-6- | |
| | azaspiro[3.3]heptan-6-yl)quinoxalin-5- | |
| | yl)ethyl)amino)picolinic acid | |
| 146 | 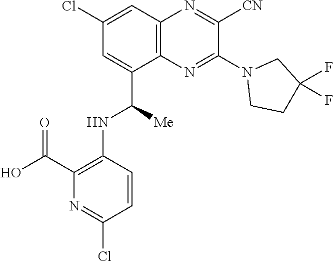 [SMILES: C[C@@H](Nc1ccc(Cl)nc1C(=O)O)c1cc(Cl)cc2nc(C#N)c(N3CCC(F)(F)C3)nc12] | ¹H NMR (400 MHz, methanol- d₄) δ = 7.79 (d, J = 2.4 Hz, 1H), 7.68 (d, J = 2.4 Hz, 1H), 7.20 (d, J = 8.8 Hz, 1H), 7.00 (d, J = 9.2 Hz, 1H), 5.49 (q, J = 6.4 Hz, 1H), 4.42-4.15 (m, 4H), 2.66- 2.52 (m, 2H), 1.74 (d, J = 6.4 Hz, 3H). LCMS [M + 1]⁺ = 493.1. |
| | (R)-6-chloro-3-((1-(7-chloro-2-cyano-3-(3,3- | |
| | difluoropyrrolidin-1-yl)quinoxalin-5- | |
| | yl)ethyl)amino)picolinic acid | |
| 147 and 148 | 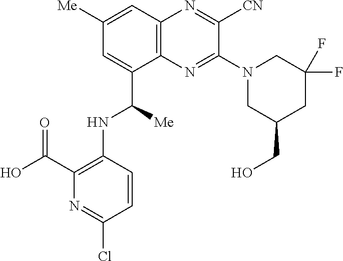 [SMILES: Cc1cc([C@@H](C)Nc2ccc(Cl)nc2C(=O)O)c2nc(N3C[C@H](CO)CC(F)(F)C3)c(C#N)nc2c1] | Separated isomers by SFC before saponification: Final product from saponification of first eluting methyl ester isomer (147): ¹H NMR (400 MHz, methanol-d₄) δ = 7.66 (d, J = 4.0 Hz, 2H), 7.19 (d, J = 9.2 Hz, 1H), 7.03 (d, J = 9.2 Hz, 1H), 5.57 (q, J = 6.8 Hz, 1H), 4.58-4.45 (m, 2H), 3.69- 3.53 (m, 3H), 3.06 (dd, J = 11.2, 12.8 Hz, 1H), 2.46 (s, 3H), 2.45- 2.25 (m, 2H), 1.98-1.80 (m, 1H), 1.74 (d, J = 6.8 Hz, 3H). LCMS [M + 23]⁺ = 539.1. Final product from |
| | 6-chloro-3-(((R)-1-(2-cyano-3-((R)-3,3-difluoro-5- | saponification of second eluting |
| | (hydroxymethyl)piperidin-1-yl)-7- | methyl ester isomer (148): ¹H |
| | methylquinoxalin-5-yl)ethyl)amino)picolinic acid | NMR (400 MHz, methanol-d₄) |
| | AND | δ = 7.66 (s, 2H), 7.18 (d, J = 9.2 |
| | 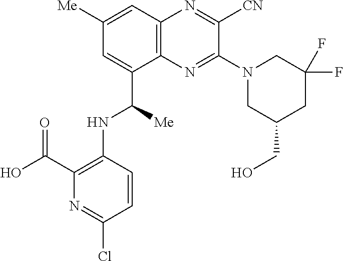 [SMILES: Cc1cc([C@@H](C)Nc2ccc(Cl)nc2C(=O)O)c2nc(N3C[C@@H](CO)CC(F)(F)C3)c(C#N)nc2c1] | Hz, 1H), 6.99 (d, J = 9.2 Hz, 1H), 5.61 (q, J = 6.8 Hz, 1H), 4.55-4.49 (m, 2H), 3.67-3.60 (m, 2H), 3.60-3.48 (m, 1H), 3.11-3.05 (m, 1H), 2.46 (s, 3H), 2.43-2.42 (m, 1H), 2.32- 2.30 (m, 1H), 1.94-1.89 (m, 1H), 1.71 (d, J = 6.8 Hz, 3H). LCMS [M + 23]⁺ = 539.1. |
| | 6-chloro-3-(((R)-1-(2-cyano-3-((S)-3,3-difluoro-5- | |
| | (hydroxymethyl)piperidin-1-yl)-7- | |
| | methylquinoxalin-5-yl)ethyl)amino)picolinic acid | |
| 149 and 150 | 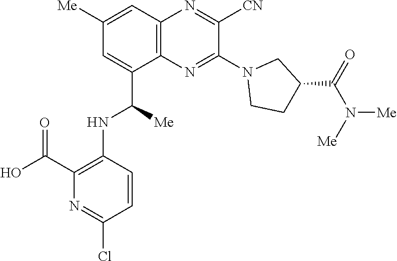 [SMILES: Cc1cc([C@@H](C)Nc2ccc(Cl)nc2C(=O)O)c2nc(N3CC[C@@H](C(=O)N(C)C)C3)c(C#N)nc2c1] | Separated isomers by SFC before saponification: Final product from saponification of first eluting methyl ester isomer (149): ¹H NMR (400 MHz, methanol-d₄) δ = 7.57 (d, J = 2.0 Hz, 1H), 7.53 (s, 1H), 7.18 (d, J = 9.2 Hz, 1H), 7.01 (d, J = 8.8 Hz, 1H), 5.51 (q, J = 6.8 Hz, 1H), 4.11-4.09 (m, 2H), 4.04-4.02 (m, 2H), 3.67 (quin, J = 6.8 Hz, 1H), 3.20 (s, 3H), 2.99 (s, 3H), 2.41 (s, 3H), 2.39-2.35 (m, 1H), 2.27-2.22 (m, 1H), 1.70 (d, J = 6.8 Hz, 3H). LCMS |
| | 6-chloro-3-(((R)-1-(2-cyano-3-((R)-3- | [M + 1]⁺ = 508.2. |
| | (dimethylcarbamoyl)pyrrolidin-1-yl)-7- | Final product from |
| | methylquinoxalin-5-yl)ethyl)amino)picolinic acid | saponification of second |
| | AND | eluting methyl ester isomer |
| | 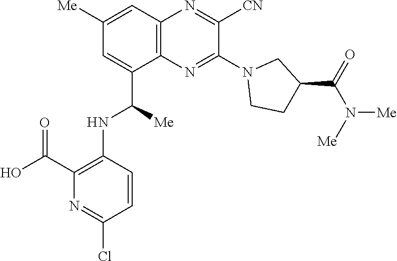 [SMILES: Cc1cc([C@@H](C)Nc2ccc(Cl)nc2C(=O)O)c2nc(N3CC[C@H](C(=O)N(C)C)C3)c(C#N)nc2c1] | (150): ¹H NMR (400 MHz, methanol-d₄) δ = 7.57 (d, J = 1.6 Hz, 1H), 7.52 (s, 1H), 7.14 (d, J = 8.8 Hz, 1H), 6.97 (d, J = 8.8 Hz, 1H), 5.50 (q, J = 6.4 Hz, 1H), 4.19-4.15 (m, 2H), 4.08-4.01 (m, 2H), 3.67 (quin, J = 6.8 Hz, 1H), 3.21 (s, 3H), 2.99 (s, 3H), 2.41 (s, 3H), 2.41- 2.36 (m, 1H), 2.31-2.20 (m, 1H), 1.69 (d, J = 6.8 Hz, 3H). LCMS [M + 1]⁺ = 508.1. |
| | 6-chloro-3-(((R)-1-(2-cyano-3-((S)-3- | |
| | (dimethylcarbamoyl)pyrrolidin-1-yl)-7- | |
| | methylquinoxalin-5-yl)ethyl)amino)picolinic acid | |
| 151 | 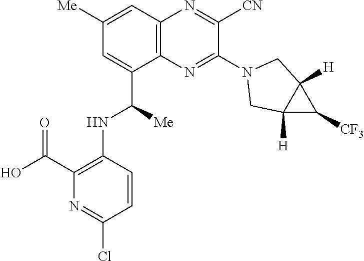 [SMILES: Cc1cc([C@@H](C)Nc2ccc(Cl)nc2C(=O)O)c2nc(N3C[C@@H]4[C@H](C3)[C@H]4C(F)(F)F)c(C#N)nc2c1] | ¹H NMR (400 MHz, methanol- d₄) δ = 7.59-7.54 (m, 2H), 7.20 (d, J = 9.2 Hz, 1H), 7.01 (d, J = 8.8 Hz, 1H), 5.50 (q, J = 6.8 Hz, 1H), 4.45 (dd, J = 8.4, 11.2 Hz, 2H), 3.96 (t, J = 13.6 Hz, 2H), 2.42 (s, 3H), 2.28 (t, J = 3.2 Hz, 2H), 1.80-1.73 (m, 1H), 1.71 (d, J = 6.8 Hz, 3H). LCMS [M + 23]⁺ = 539.1. |
| | 6-chloro-3-(((R)-1-(2-cyano-7-methyl-3- | |
| | ((1R,5S,6R)-6-(trifluoromethyl)-3- | |
| | azabicyclo[3.1.0]hexan-3-yl)quinoxalin-5- | |
| | yl)ethyl)amino)picolinic acid | |
| 152 | 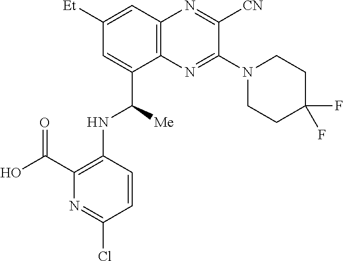 [SMILES: CCc1cc([C@@H](C)Nc2ccc(Cl)nc2C(=O)O)c2nc(N3CCC(F)(F)CC3)c(C#N)nc2c1] | ¹H NMR (400 MHz, CDCl₃) δ = 8.39 (br d, J = 6.0 Hz, 1H), 7.72 (s, 1H), 7.60 (d, J = 1.6 Hz, 1H), 7.13 (d, J = 8.8 Hz, 1H), 6.77 (d, J = 8.8 Hz, 1H), 5.50 (quin, J = 6.8 Hz, 1H), 3.97-3.84 (m, 4H), 2.79 (q, J = 7.6 Hz, 2H), 2.34-2.20 (m, 4H), 1.74 (d, J = 6.8 Hz, 3H), 1.28 (t, J = 7.6 Hz, 3H). LCMS [M + 23]⁺ = 523.1 |
| | (R)-6-chloro-3-((1-(2-cyano-3-(4,4- | |
| | difluoropiperidin-1-yl)-7-ethylquinoxalin-5- | |
| | yl)ethyl)amino)picolinic acid | |
| 153 | 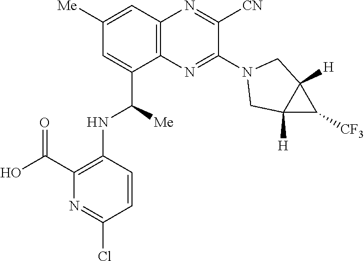 [SMILES: Cc1cc([C@@H](C)Nc2ccc(Cl)nc2C(=O)O)c2nc(N3C[C@@H]4[C@H](C3)[C@@H]4C(F)(F)F)c(C#N)nc2c1] | ¹H NMR (400 MHz, methanol- d₄) δ = 7.57 (d, J = 1.6 Hz, 1H), 7.54 (s, 1H), 7.16 (d, J = 9.2 Hz, 1H), 6.98 (d, J = 9.2 Hz, 1H), 5.50 (q, J = 6.8 Hz, 1H), 4.39 (d, J = 12.0 Hz, 2H), 4.20 (br d, J = 10.0 Hz, 2H), 2.41 (s, 3H), 2.36 (br dd, J = 2.0, 8.0 Hz, 2H), 2.01-1.86 (m, 1H), 1.70 (d, J = 6.4 Hz, 3H). LCMS [M + 23]⁺ = 539.2. |
| | 6-chloro-3-(((R)-1-(2-cyano-7-methyl-3- | |
| | ((1R,5S,6S)-6-(trifluoromethyl)-3- | |
| | azabicyclo[3.1.0]hexan-3-yl)quinoxalin-5- | |
| | yl)ethyl)amino)picolinic acid | |
| 154 | 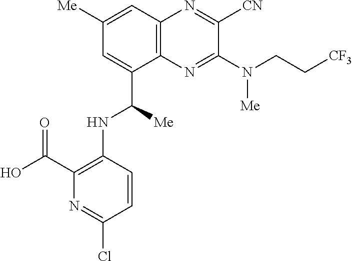 [SMILES: Cc1cc([C@@H](C)Nc2ccc(Cl)nc2C(=O)O)c2nc(N(C)CCC(F)(F)F)c(C#N)nc2c1] | ¹H NMR (400 MHz, methanol- d₄) δ = 7.60 (s, 2H), 7.17 (d, J = 9.2 Hz, 1H), 6.92 (d, J = 8.8 Hz, 1H), 5.55 (q, J = 6.8 Hz, 1H), 4.11-4.04 (m, 2H), 3.52 (s, 3H), 2.81-2.66 (m, 2H), 2.43 (s, 3H), 1.68 (d, J = 6.8 Hz, 3H). LCMS [M + 23]⁺ = 515.2. |
| | (R)-6-chloro-3-((1-(2-cyano-7-methyl-3- | |
| | (methyl(3,3,3-trifluoropropyl)amino)quinoxalin-5- | |
| | yl)ethyl)amino)picolinic acid | |
| 155 and 156 | 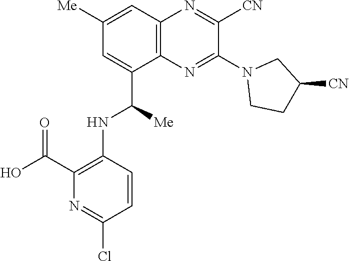 [SMILES: Cc1cc([C@@H](C)Nc2ccc(Cl)nc2C(=O)O)c2nc(N3CC[C@H](C#N)C3)c(C#N)nc2c1] | Separated isomers by SFC before saponification: Final product from saponification of first eluting methyl ester isomer (155): ¹H NMR (400 MHz, methanol-d₄) δ = 7.59 (s, 1H), 7.57 (s, 1H), 7.19 (d, J = 9.2 Hz, 1H), 7.03 (d, J = 9.2 Hz, 1H), 5.51 (q, J = 6.4 Hz, 1H), 4.32-4.04 (m, 4H), 3.55 (quin, J = 6.4 Hz, 1H), 2.58-2.48 (m, 1H), 2.47-2.37 (m, 4H), 1.73 (d, J = 6.4 Hz, 3H). LCMS [M + 1]⁺ = 462.2. Final product from saponification of second eluting |
| | 6-chloro-3-(((R)-1-(2-cyano-3-((S)-3- | methyl ester isomer (156): ¹H |
| | cyanopyrrolidin-1-yl)-7-methylquinoxalin-5- | NMR (400 MHz, methanol-d₄) |
| | yl)ethyl)amino)picolinic acid | δ = 7.59 (s, 1H), 7.56 (s, 1H), |
| | AND | 7.19 (d, J = 9.2 Hz, 1H), 7.03 |
| | 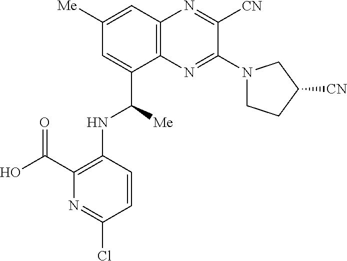 [SMILES: Cc1cc([C@@H](C)Nc2ccc(Cl)nc2C(=O)O)c2nc(N3CC[C@@H](C#N)C3)c(C#N)nc2c1] | (d, J = 9.2 Hz, 1H), 5.51 (q, J = 6.4 Hz, 1H), 4.25-4.06 (m, 4H), 3.57-3.54 (m, 1H), 2.54- 2.49(m, 1H), 2.47-2.38 (m, 4H), 1.72 (d, J = 6.8 Hz, 3H). LCMS [M + 1]⁺ = 462.1. |
| | 6-chloro-3-(((R)-1-(2-cyano-3-((R)-3- | |
| | cyanopyrrolidin-1-yl)-7-methylquinoxalin-5- | |
| | yl)ethyl)amino)picolinic acid | |
| 157 | 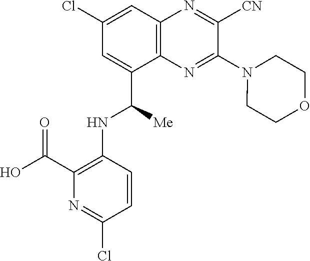 [SMILES: C[C@@H](Nc1ccc(Cl)nc1C(=O)O)c1cc(Cl)cc2nc(C#N)c(N3CCOCC3)nc12] | ¹H NMR (400 MHz, methanol- d₄) δ = 7.89 (d, J = 2.4 Hz, 1H), 7.75 (d, J = 2.0 Hz, 1H), 7.25 (d, J = 8.8 Hz, 1H), 7.03 (d, J = 9.2 Hz, 1H), 5.55 (q, J = 6.8 Hz, 1H), 3.96-3.91 (m, 4H), 3.90- 3.81 (m, 4H), 1.76 (d, J = 6.8 Hz, 3H). LCMS [M + 23]⁺ = 495.1 |
| | (R)-6-chloro-3-((1-(7-chloro-2-cyano-3- | |
| | morpholinoquinoxalin-5-yl)ethyl)amino)picolinic | |
| | acid | |
| 158 | 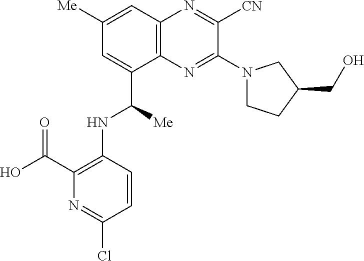 [SMILES: Cc1cc([C@@H](C)Nc2ccc(Cl)nc2C(=O)O)c2nc(N3CC[C@H](CO)C3)c(C#N)nc2c1] | ¹H NMR (400 MHz, methanol- d₄) δ = 7.54 (d, J = 3.2 Hz, 2H), 7.20 (d, J = 9.2 Hz, 1H), 7.02 (d, J = 9.2 Hz, 1H), 5.52 (q, J = 6.8 Hz, 1H), 4.13-4.03 (m, 2H), 3.91 (td, J = 7.6, 10.8 Hz, 1H), 3.75-3.59 (m, 3H), 2.67- 2.55 (m, 1H), 2.41 (s, 3H), 2.27- 2.16 (m, 1H), 1.99-1.85 (m, 1H), 1.71 (d, J = 6.4 Hz, 3H). LCMS [M + 1]⁺ = 467.3. |
| | 6-chloro-3-(((R)-1-(2-cyano-3-((S)-3- | |
| | (hydroxymethyl)pyrrolidin-1-yl)-7- | |
| | methylquinoxalin-5-yl)ethyl)amino)picolinic acid | |
| 159 | 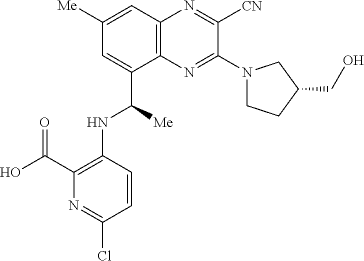 [SMILES: Cc1cc([C@@H](C)Nc2ccc(Cl)nc2C(=O)O)c2nc(N3CC[C@@H](CO)C3)c(C#N)nc2c1] | ¹H NMR (400 MHz, methanol- d₄) δ = 7.55-7.53 (m, 2H), 7.19 (d, J = 8.8 Hz, 1H), 7.01 (d, J = 9.2 Hz, 1H), 5.56-5.47 (m, 1H), 4.07-4.00 (m, 2H), 4.00- 3.93 (m, 1H), 3.78-3.73 (m, 2H), 3.71-3.62 (m, 1H), 2.60 (td, J = 6.8, 14.0 Hz, 1H), 2.41 (s, 3H), 2.22-2.19 (m, 1H), 1.95-1.90 (m, 1H), 1.71 (d, J = 6.4 Hz, 3H). LCMS [M + 1]⁺ = 467.1. |
| | 6-chloro-3-(((R)-1-(2-cyano-3-((R)-3- | |
| | (hydroxymethyl)pyrrolidin-1-yl)-7- | |
| | methylquinoxalin-5-yl)ethyl)amino)picolinic acid | |
| 160 | 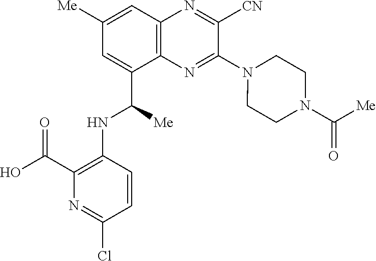 [SMILES: CC(=O)N1CCN(c2nc3c([C@@H](C)Nc4ccc(Cl)nc4C(=O)O)cc(C)cc3nc2C#N)CC1] | ¹H NMR (400 MHz, methanol- d₄) δ = 7.68 (s, 2H), 7.21 (d, J = 8.8 Hz, 1H), 7.05 (d, J = 9.2 Hz, 1H), 5.53 (q, J = 6.8 Hz, 1H), 3.90-3.76 (m, 8H), 2.48 (s, 3H), 2.19 (s, 3H), 1.74 (d, J = 6.4 Hz, 3H). LCMS [M + 1]⁺ = 494.0. |
| | (R)-3-((1-(3-(4-acetylpiperazin-1-yl)-2-cyano-7- | |
| | methylquinoxalin-5-yl)ethyl)amino)-6- | |
| | chloropicolinic acid | |
| 161 | 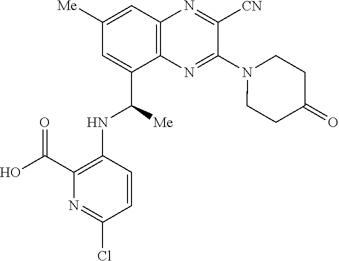 [SMILES: Cc1cc([C@@H](C)Nc2ccc(Cl)nc2C(=O)O)c2nc(N3CCC(=O)CC3)c(C#N)nc2c1] | ¹H NMR (400 MHz, chloroform- d) δ = 8.43-8.41 (m, 1H), 7.70 (s, 1H), 7.57 (d, J = 1.6 Hz, 1H), 7.11 (d, J = 8.8 Hz, 1H), 6.76 (d, J = 8.8 Hz, 1H), 5.50-5.43 (m, 1H), 4.11 (t, J = 6.0 Hz, 4H), 2.77 (t, J = 6.0 Hz, 4H), 2.49 (s, 3H), 1.72 (d, J = 6.4 Hz, 3H). LCMS [M + 1]⁺ = 465.3. |
| | (R)-6-chloro-3-((1-(2-cyano-7-methyl-3-(4- | |
| | oxopiperidin-1-yl)quinoxalin-5- | |
| | yl)ethyl)amino)picolinic acid | |
| 162 | 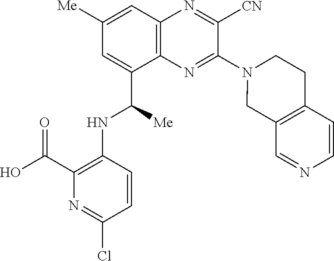 [SMILES: Cc1cc([C@@H](C)Nc2ccc(Cl)nc2C(=O)O)c2nc(N3CCc4ccncc4C3)c(C#N)nc2c1] | ¹H NMR (400 MHz, methanol- d₄) δ = 8.51 (s, 1H), 8.33 (d, J = 5.2 Hz, 1H), 7.69 (d, J = 1.6 Hz, 1H), 7.65 (s, 1H), 7.32 (d, J = 5.2 Hz, 1H), 7.08 (d, J = 9.2 Hz, 1H), 6.91 (d, J = 8.8 Hz, 1H), 5.56-5.50 (m, 1H), 4.98-4.96 (m, 2H), 4.16-4.12 (m, 2H), 3.21-3.17 (m, 2H), 2.46 (s, 3H), 1.69 (d, J = 6.8 Hz, 3H). LCMS [M + 1]⁺ = 500.2. |
| | (R)-6-chloro-3-((1-(2-cyano-3-(3,4-dihydro-2,7- | |
| | naphthyridin-2(1H)-yl)-7-methylquinoxalin-5- | |
| | yl)ethyl)amino)picolinic acid | |
| 163 | 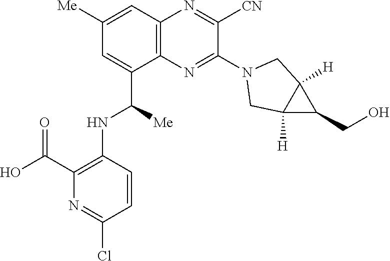 [SMILES: Cc1cc([C@@H](C)Nc2ccc(Cl)nc2C(=O)O)c2nc(N3C[C@@H]4[C@H](CO)[C@@H]4C3)c(C#N)nc2c1] | ¹H NMR (400 MHz, methanol- d₄) δ = 7.58 (s, 1H), 7.55 (s, 1H), 7.20 (d, J = 9.2 Hz, 1H), 7.02 (d, J = 8.8 Hz, 1H), 5.50 (q, J = 6.8 Hz, 1H), 4.24-4.08 (m, 4H), 3.62-3.32 (m, 2H), 2.43 (s, 3H), 2.08-2.04 (m, 2H), 1.72 (d, J = 6.4 Hz, 3H), 1.40-1.30 (m, 1H). LCMS [M + 1]⁺ = 479.2. |
| | 6-chloro-3-(((R)-1-(2-cyano-3-((1R,5S,6S)-6- | |
| | (hydroxymethyl)-3-azabicyclo[3.1.0]hexan-3-yl)- | |
| | 7-methylquinoxalin-5-yl)ethyl)amino)picolinic | |
| | acid | |
| 164 and 165 | 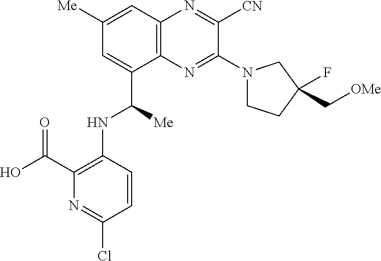 [SMILES: COC[C@@]1(F)CCN(c2nc3c([C@@H](C)Nc4ccc(Cl)nc4C(=O)O)cc(C)cc3nc2C#N)C1] | Separated isomers by SFC before saponification: Final product from saponification of first eluting methyl ester isomer (164): ¹H NMR (400 MHz, methanol-d₄) δ = 7.57 (d, J = 2.4 Hz, 2H), 7.20 (d, J = 8.8 Hz, 1H), 7.02 (d, J = 9.2 Hz, 1H), 5.55-5.46 (m, 1H), 4.26-4.07 (m, 4H), 3.83- 3.71 (m, 2H), 3.48 (s, 3H), 2.43 (s, 3H), 2.40-2.19 (m, 2H), 1.72 (d, J = 6.8 Hz, 3H). LCMS [M + 1]⁺ = 499.0. Final product from saponification of second eluting |
| | 6-chloro-3-(((R)-1-(2-cyano-3-((R)-3-fluoro-3- | methyl ester isomer (165): ¹H |
| | (methoxymethyl)pyrrolidin-1-yl)-7- | NMR (400 MHz, methanol-d₄) |
| | methylquinoxalin-5-yl)ethyl)amino)picolinic acid | δ = 7.57 (br d, J = 2.4 Hz, 2H), |
| | AND | 7.20 (d, J = 9.2 Hz, 1H), 7.03 |
| | 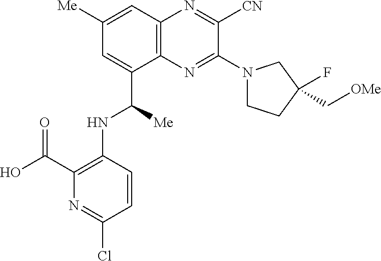 [SMILES: COC[C@]1(F)CCN(c2nc3c([C@@H](C)Nc4ccc(Cl)nc4C(=O)O)cc(C)cc3nc2C#N)C1] | (d, J = 9.2 Hz, 1H), 5.53 (q, J = 6.8 Hz, 1H), 4.29-4.01 (m, 4H), 3.74 (d, J = 4.8 Hz, 1H), 3.83-3.70 (m, 1H), 3.48 (s, 3H), 2.43 (s, 3H), 2.39-2.20 (m, 2H), 1.71 (d, J = 6.4 Hz, 3H). LCMS [M + 1]⁺ = 499.1. |
| | 6-chloro-3-(((R)-1-(2-cyano-3-((S)-3-fluoro-3- | |
| | (methoxymethyl)pyrrolidin-1-yl)-7- | |
| | methylquinoxalin-5-yl)ethyl)amino)picolinic acid | |
| 166 | 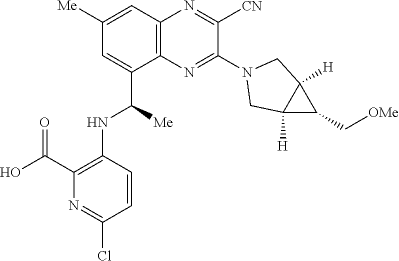 [SMILES: COC[C@@H]1[C@H]2CN(c3nc4c([C@@H](C)Nc5ccc(Cl)nc5C(=O)O)cc(C)cc4nc3C#N)C[C@@H]12] | ¹H NMR (400 MHz, methanol- d₄) δ = 7.55 (d, J = 1.6 Hz, 1H), 7.51 (s, 1H), 7.19 (d, J = 8.8 Hz, 1H), 6.99 (d, J = 9.2 Hz, 1H), 5.49 (q, J = 6.4 Hz, 1H), 4.35- 4.30 (m, 2H), 3.94-3.87 (m, 2H), 3.39-3.31 (m, 5H), 2.40 (s, 3H), 1.78 (br s, 2H), 1.70 (d, J = 6.8 Hz, 3H), 1.06-1.01 (m, 1H). LCMS [M + 1]⁺ = 493.2. |
| | 6-chloro-3-(((R)-1-(2-cyano-3-((1R,5S,6R)-6- | |
| | (methoxymethyl)-3-azabicyclo[3.1.0]hexan-3-yl)- | |
| | 7-methylquinoxalin-5-yl)ethyl)amino)picolinic | |
| | acid | |
| 167 | 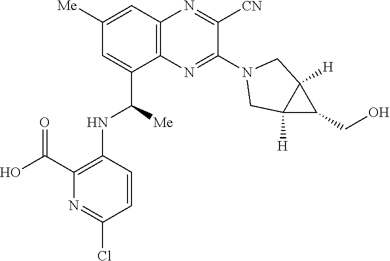 [SMILES: Cc1cc([C@@H](C)Nc2ccc(Cl)nc2C(=O)O)c2nc(N3C[C@@H]4[C@@H](CO)[C@@H]4C3)c(C#N)nc2c1] | ¹H NMR (400 MHz, methonal- d₄) δ = 7.64-7.44 (m, 2H), 7.20 (d, J = 8.8 Hz, 1H), 7.00 (br d, J = 9.2 Hz, 1H), 5.50 (q, J = 6.4 Hz, 1H), 4.35 (br t, J = 10.4 Hz, 2H), 3.96-3.87 (m, 2H), 3.53 (d, J = 6.8 Hz, 2H), 2.43 (s, 3H), 1.79 (br s, 2H), 1.72 (d, J = 6.8 Hz, 3H), 1.04-1.01 (m, 1H). LCMS [M + 23]⁺ = 501.2. |
| | 6-chloro-3-(((R)-1-(2-cyano-3-((1R,5S,6R)-6- | |
| | (hydroxymethyl)-3-azabicyclo[3.1.0]hexan-3-yl)- | |
| | 7-methylquinoxalin-5-yl)ethyl)amino)picolinic | |
| | acid | |
| 168 | 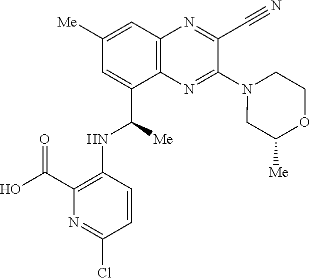 [SMILES: Cc1cc([C@@H](C)Nc2ccc(Cl)nc2C(=O)O)c2nc(N3CCO[C@H](C)C3)c(C#N)nc2c1] | ¹H NMR (400 MHz, methanol- d₄) δ = 7.67-7.62 (m, 2H), 7.21 (d, J = 8.8 Hz, 1H), 7.05 (d, J = 9.2 Hz, 1H), 5.51 (q, J = 6.4 Hz, 1H), 4.27 (br d, J = 12.8 Hz, 1H), 4.21 (br d, J = 13.2 Hz, 1H), 4.04 (dd, J = 1.6, 11.6 Hz, 1H), 3.93-3.77 (m, 2H), 3.30- 3.26 (m, 1H), 2.91 (dd, J = 10.4, 12.8 Hz, 1H), 2.46 (s, 3H), 1.74 (d, J = 6.8 Hz, 3H), 1.27 (d, J = 6.0 Hz, 3H). LCMS [M + 23]⁺ = 489.1. |
| | 6-chloro-3-(((R)-1-(2-cyano-7-methyl-3-((R)-2- | |
| | methylmorpholino)quinoxalin-5- | |
| | yl)ethyl)amino)picolinic acid | |
| 169 and 170 | 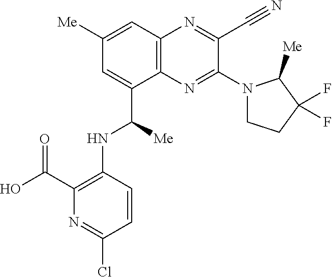 [SMILES: Cc1cc([C@@H](C)Nc2ccc(Cl)nc2C(=O)O)c2nc(N3CCC(F)(F)[C@@H]3C)c(C#N)nc2c1] | Separated isomers by SFC before saponification: Final product from saponification of first eluting methyl ester isomer (169): ¹H NMR (400 MHz, methanol-d₄) δ = 7.61 (d, J = 1.6 Hz, 1H), 7.55 (s, 1H), 7.18 (d, J = 9.2 Hz, 1H), 6.98 (d, J = 9.2 Hz, 1H), 5.49 (q, J = 6.8 Hz, 1H), 4.84-4.80 (m, 1H), 4.34 (m, 1H), 4.06 (m, 1H), 2.69-2.53 (m, 2H), 2.41 (s, 3H), 1.72 (d, J = 6.4 Hz, 3H), 1.44 (dd, J = 2.8, 6.8 Hz, 3H). LCMS [M + 23]⁺ = 509.1. Final product from saponification of second eluting |
| | 6-chloro-3-(((R)-1-(2-cyano-3-((S)-3,3-difluoro-2- | methyl ester isomer (170): ¹H |
| | methylpyrrolidin-1-yl)-7-methylquinoxalin-5- | NMR (400 MHz, methanol-d₄) |
| | yl)ethyl)amino)picolinic acid | δ = 7.59 (s, 1H), 7.56 (s, 1H), |
| | AND | 7.14 (d, J = 9.2 Hz, 1H), 6.89 |
| | 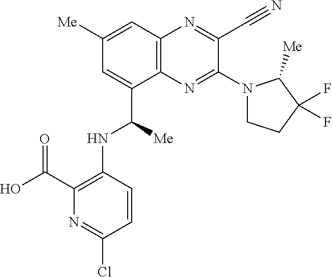 [SMILES: Cc1cc([C@@H](C)Nc2ccc(Cl)nc2C(=O)O)c2nc(N3CCC(F)(F)[C@H]3C)c(C#N)nc2c1] | (d, J = 8.8 Hz, 1H), 5.58 (q, J = 6.8 Hz, 1H), 4.77-4.69 (m, 1H), 4.39-4.29 (m, 1H), 4.13- 4.04 (m, 1H), 2.67-2.53 (m, 2H), 2.41 (s, 3H), 1.66 (d, J = 6.8 Hz, 3H), 1.45 (dd, J = 2.8, 6.8 Hz, 3H). LCMS [M + 23]⁺ = 509.1. |
| | 6-chloro-3-(((R)-1-(2-cyano-3-((R)-3,3-difluoro-2- | |
| | methylpyrrolidin-1-yl)-7-methylquinoxalin-5- | |
| | yl)ethyl)amino)picolinic acid | |
| 171 and 172 | 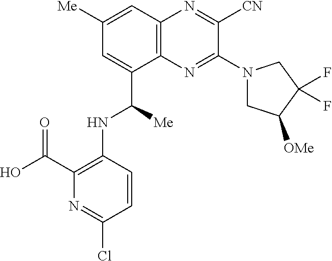 [SMILES: CO[C@H]1CN(c2nc3c([C@@H](C)Nc4ccc(Cl)nc4C(=O)O)cc(C)cc3nc2C#N)CC1(F)F] | Separated isomers by SFC before saponification: Final product from saponification of first eluting methyl ester isomer (171): ¹H NMR (400 MHz, methanol-d₄) δ = 7.65-7.55 (m, 2H), 7.21 (d, J. = 8.8 Hz, 1H), 7.05 (d, J. = 9.2 Hz, 1H), 5.51-5.42 (m, 1H), 4.38-4.19 (m, 4H), 4.15- 4.05 (m, 1H), 3.60 (s, 3H), 2.43 (s, 3H), 1.73 (d, J. = 6.4 Hz, 3H). LCMS [M + 23]⁺ = 525.1. Final product from saponification of second eluting methyl ester isomer (172): ¹H |
| | 6-chloro-3-(((R)-1-(2-cyano-3-((S)-3,3-difluoro-4- | NMR (400 MHz, methanol-d₄) |
| | methoxypyrrolidin-1-yl)-7-methylquinoxalin-5- | δ = 7.64-7.56 (m, 2H), 7.20 (d, |
| | yl)ethyl)amino)picolinic acid | J. = 8.8 Hz, 1H), 7.02 (d, J. = |
| | AND | 9.2 Hz, 1H), 5.51 (q, J = 6.8 Hz, |
| | 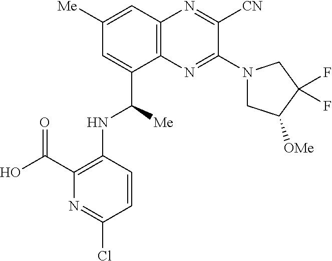 [SMILES: CO[C@@H]1CN(c2nc3c([C@@H](C)Nc4ccc(Cl)nc4C(=O)O)cc(C)cc3nc2C#N)CC1(F)F] | 1H), 4.36-4.16 (m, 4H), 4.06 (dt, J. = 2.4, 12.0 Hz, 1H), 3.60 (s, 3H), 2.44 (s, 3H), 1.73 (d, J. = 6.8 Hz, 3H). LCMS [M + 1]⁺ = 503.2. |
| | 6-chloro-3-(((R)-1-(2-cyano-3-((R)-3,3-difluoro-4- | |
| | methoxypyrrolidin-1-yl)-7-methylquinoxalin-5- | |
| | yl)ethyl)amino)picolinic acid | |
| 173 and 174 | 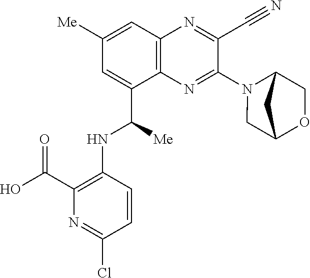 [SMILES: Cc1cc([C@@H](C)Nc2ccc(Cl)nc2C(=O)O)c2nc(N3C[C@@H]4C[C@H]3CO4)c(C#N)nc2c1] | ¹H NMR (400 MHz, methanol- d₄) δ = 7.55 (d, J = 2.0 Hz, 1H), 7.47 (s, 1H), 7.16 (d, J = 8.8 Hz, 1H), 6.97 (d, J = 9.2 Hz, 1H), 5.50 (m, 1H), 5.28 (s, 1H), 4.77 (s, 1H), 4.08 (dd, J = 1.2, 10.0 Hz, 1H), 4.04-4.02 (m, 1H), 3.99-3.95 (m, 1H), 3.76 (d, J = 10.0 Hz, 1H), 2.38 (s, 3H), 2.12- 2.03 (m, 2H), 1.67 (d, J = 6.8 Hz, 3H). LCMS [M + 1]⁺ = 465.2. AND ¹H NMR (400 MHz, methanol- d₄) δ = 7.63 (d, J = 1.6 Hz, 1H), 7.59 (s, 1H), 7.23 (d, J = 9.2 Hz, 1H), 7.08 (d, J = 8.8 Hz, 1H), 5.49 (q, J = 6.8 Hz, 1H), 5.37 (s, |
| | 3-(((R)-1-(3-((1S,4S)-2-oxa-5- | 1H), 4.80 (br s, 1H), 4.15-4.04 |
| | azabicyclo[2.2.1]heptan-5-yl)-2-cyano-7- | (m, 2H), 3.98-3.93 (m, 1H), |
| | methylquinoxalin-5-yl)ethyl)amino)-6- | 3.81 (d, J = 10.0 Hz, 1H), 2.46 |
| | chloropicolinic acid | (s, 3H), 2.18-2.03 (m, 2H), |
| | AND | 1.73 (d, J = 6.4 Hz, 3H). LCMS |
| | 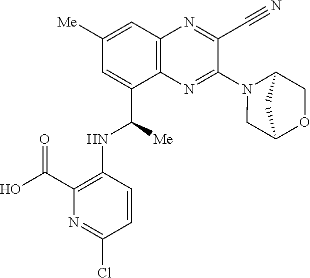 [SMILES: Cc1cc([C@@H](C)Nc2ccc(Cl)nc2C(=O)O)c2nc(N3C[C@H]4C[C@@H]3CO4)c(C#N)nc2c1] | [M + 1]⁺ = 465.3. |
| | 3-(((R)-1-(3-((1R,4R)-2-oxa-5- | |
| | azabicyclo[2.2.1]heptan-5-yl)-2-cyano-7- | |
| | methylquinoxalin-5-yl)ethyl)amino)-6- | |
| | chloropicolinic acid | |
| 175 | 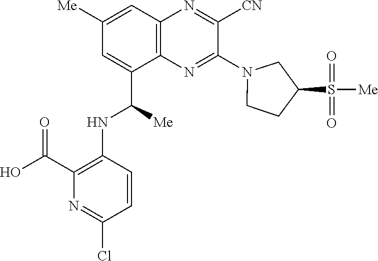 [SMILES: Cc1cc([C@@H](C)Nc2ccc(Cl)nc2C(=O)O)c2nc(N3CC[C@H](S(C)(=O)=O)C3)c(C#N)nc2c1] | ¹H NMR (400 MHz, methanol- d₄) δ = 7.62 (d, J = 2.0 Hz, 1H), 7.57 (s, 1H), 7.19 (d, J = 8.8 Hz, 1H), 7.05 (d, J = 8.8 Hz, 1H), 5.54 (q, J = 6.8 Hz, 1H), 4.45- 4.22 (m, 3H), 4.20-4.07 (m, 2H), 3.12 (s, 3H), 2.73-2.55 (m, 2H), 2.44 (s, 3H), 1.74 (d, J = 6.8 Hz, 3H). LCMS [M + 1]⁺ = 515.1. |
| | 6-chloro-3-(((R)-1-(2-cyano-7-methyl-3-((S)-3- | |
| | (methylsulfonyl)pyrrolidin-1-yl)quinoxalin-5- | |
| | yl)ethyl)amino)picolinic acid | |
| 176 | 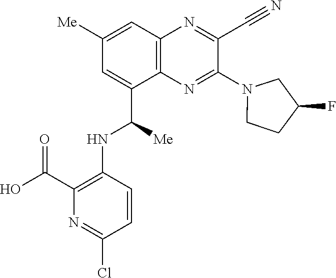 [SMILES: Cc1cc([C@@H](C)Nc2ccc(Cl)nc2C(=O)O)c2nc(N3CC[C@H](F)C3)c(C#N)nc2c1] | ¹H NMR (400 MHz, methanol- d₄) δ = 7.57 (d, J = 2.0 Hz, 1H), 7.55 (d, J = 0.8 Hz, 1H), 7.20 (d, J = 8.8 Hz, 1H), 7.02 (d, J = 9.2 Hz, 1H), 5.55-5.37 (m, 2H), 4.27-4.16 (m, 2H), 4.15- 4.01 (m, 2H), 2.49-2.21 (m, 5H), 1.73 (d, J = 6.8 Hz, 3H). LCMS [M + 1]⁺ = 455.2. |
| | 6-chloro-3-(((R)-1-(2-cyano-3-((S)-3- | |
| | fluoropyrrolidin-1-yl)-7-methylquinoxalin-5- | |
| | yl)ethyl)amino)picolinic acid | |
| 177 | 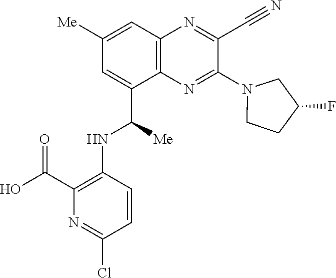 [SMILES: Cc1cc([C@@H](C)Nc2ccc(Cl)nc2C(=O)O)c2nc(N3CC[C@@H](F)C3)c(C#N)nc2c1] | ¹H NMR (400 MHz, methanol- d₄) δ = 7.56 (dd, J = 1.2, 5.6 Hz, 2H), 7.19 (d, J = 9.2 Hz, 1H), 7.02 (d, J = 9.2 Hz, 1H), 5.57- 5.37 (m, 2H), 4.28-4.18 (m, 2H), 4.17-3.97 (m, 2H), 2.47- 2.01 (m, 5H), 1.72 (d, J = 6.8 Hz, 3H). LCMS [M + 1]⁺ = 455.1. |
| | 6-chloro-3-(((R)-1-(2-cyano-3-((R)-3- | |
| | fluoropyrrolidin-1-yl)-7-methylquinoxalin-5- | |
| | yl)ethyl)amino)picolinic acid | |
| 178 | 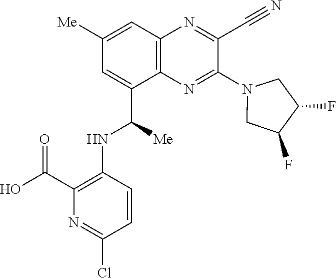 [SMILES: Cc1cc([C@@H](C)Nc2ccc(Cl)nc2C(=O)O)c2nc(N3C[C@H](F)[C@@H](F)C3)c(C#N)nc2c1] | ¹H NMR (400 MHz, CD₃OD) δ = 7.60 (s, 2H), 7.22-7.15 (m, 1H), 7.01 (d, J = 8.8 Hz, 1H), 5.57-5.47 (m, 2H), 5.42-5.36 (m, 1H), 4.43-4.19 (m, 4H), 2.44 (s, 3H), 1.74 (dd, J = 3.6, 6.8 Hz, 3H). LCMS [M + Na]⁺ = 495.1. |
| | 6-chloro-3-(((R)-1-(2-cyano-3-((3S,4S)-3,4- | |
| | difluoropyrrolidin-1-yl)-7-methylquinoxalin-5- | |
| | yl)ethyl)amino)picolinic acid | |
| 179 | 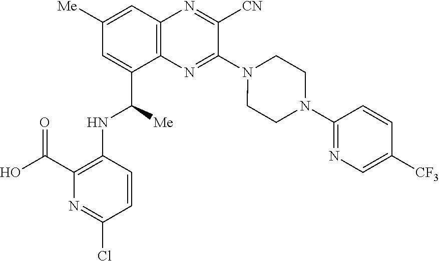 [SMILES: Cc1cc([C@@H](C)Nc2ccc(Cl)nc2C(=O)O)c2nc(N3CCN(c4ccc(C(F)(F)F)cn4)CC3)c(C#N)nc2c1] | ¹H NMR (400 MHz, DMSO-d₆) δ = 13.18-12.84 (m, 1H), 8.50 (d, J = 7.2 Hz, 1H), 8.49-8.44 (m, 1H), 7.86 (dd, J = 2.4, 8.8 Hz, 1H), 7.69 (s, 2H), 7.30 (d, J = 8.8 Hz, 1H), 7.09 (d, J = 9.2 Hz, 1H), 7.03 (d, J = 9.2 Hz, 1H), 5.48 (quin, J = 6.8 Hz, 1H), 3.98-3.88 (m, 8H), 2.44 (s, 3H), 1.68 (d, J = 6.4 Hz, 3H). LCMS [M + 23]⁺ = 597.1. |
| | (R)-6-chloro-3-((1-(2-cyano-7-methyl-3-(4-(5- | |
| | (trifluoromethyl)pyridin-2-yl)piperazin-1- | |
| | yl)quinoxalin-5-yl)ethyl)amino)picolinic acid | |
| 180 and 181 | 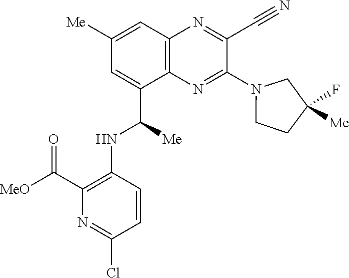 [SMILES: COC(=O)c1nc(Cl)ccc1N[C@H](C)c1cc(C)cc2nc(C#N)c(N3CC[C@@](C)(F)C3)nc12] | Separated isomers by SFC before saponification: Final product from saponification of first eluting methyl ester isomer (180): ¹H NMR (400 MHz, methanol-d₄) δ = 7.59-7.51 (m, 2H), 7.20 (d, J = 8.8 Hz, 1H), 7.03 (d, J = 8.8 Hz, 1H), 5.49 (q, J = 6.4 Hz, 1H), 4.22-3.92 (m, 4H), 2.42 (s, 3H), 2.39-2.09 (m, 2H), 1.73 (d, J = 6.8 Hz, 3H), 1.67 (d, J = 20.4 Hz, 3H). LCMS [M + 1]⁺ = 491.2. Final product from saponification of second eluting methyl ester isomer (181): ¹H |
| | methyl 6-chloro-3-(((R)-1-(2-cyano-3-((R)-3- | NMR (400 MHz, methanol-d₄) |
| | fluoro-3-methylpyrrolidin-1-yl)-7- | δ = 7.57 (s, 1H), 7.55 (s, 1H), |
| | methylquinoxalin-5-yl)ethyl)amino)picolinate | 7.19 (d, J = 9.2 Hz, 1H), 7.03 |
| | AND | (d, J = 9.2 Hz, 1H), 5.51 (q, J = |
| | 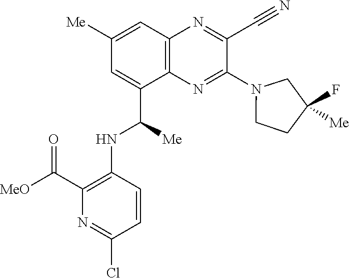 [SMILES: COC(=O)c1nc(Cl)ccc1N[C@H](C)c1cc(C)cc2nc(C#N)c(N3CC[C@](C)(F)C3)nc12] | 6.8 Hz, 1H), 4.23-3.86 (m, 4H), 2.42 (s, 3H), 2.39-2.05 (m, 2H), 1.71 (d, J = 6.4 Hz, 3H), 1.66 (d, J = 21.2 Hz, 3H). LCMS [M + 1]⁺ = 491.2. |
| | methyl 6-chloro-3-(((R)-1-(2-cyano-3-((S)-3- | |
| | fluoro-3-methylpyrrolidin-1-yl)-7- | |
| | methylquinoxalin-5-yl)ethyl)amino)picolinate | |
| 182A and 182B | 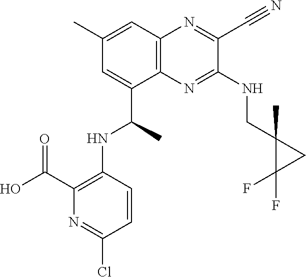 [SMILES: Cc1cc([C@@H](C)Nc2ccc(Cl)nc2C(=O)O)c2nc(NC[C@]3(C)CC3(F)F)c(C#N)nc2c1] | |
| | 6-chloro-3-(((R)-1-(2-cyano-3-((((S)-2,2-difluoro- | |
| | 1-methylcyclopropyl)methyl)amino)-7- | |
| | methylquinoxalin-5-yl)ethyl)amino)picolinic acid | |
| | AND | |
| | 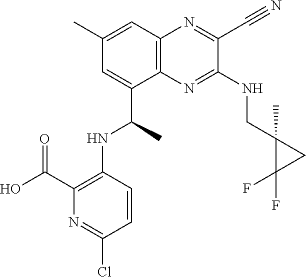 [SMILES: Cc1cc([C@@H](C)Nc2ccc(Cl)nc2C(=O)O)c2nc(NC[C@@]3(C)CC3(F)F)c(C#N)nc2c1] | |
| | 6-chloro-3-(((R)-1-(2-cyano-3-((((R)-2,2-difluoro- | |
| | 1-methylcyclopropyl)methyl)amino)-7- | |
| | methylquinoxalin-5-yl)ethyl)amino)picolinic acid | |
| 183 | 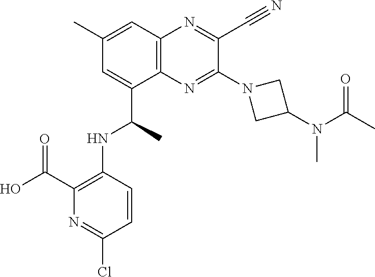 [SMILES: CC(=O)N(C)C1CN(c2nc3c([C@@H](C)Nc4ccc(Cl)nc4C(=O)O)cc(C)cc3nc2C#N)C1] | |
| | (R)-6-chloro-3-((1-(2-cyano-7-methyl-3-(3-(N- | |
| | methylacetamido)azetidin-1-yl)quinoxalin-5- | |
| | yl)ethyl)amino)picolinic acid | |
| 184 | 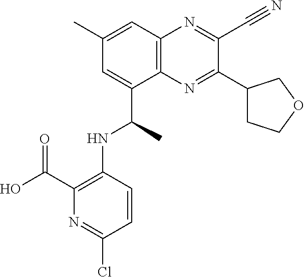 [SMILES: Cc1cc([C@@H](C)Nc2ccc(Cl)nc2C(=O)O)c2nc(C3CCOC3)c(C#N)nc2c1] | |
| | 6-chloro-3-(((1R)-1-(2-cyano-7-methyl-3- | |
| | (tetrahydrofuran-3-yl)quinoxalin-5- | |
| | yl)ethyl)amino)picolinic acid | |
| 185A and 185B | 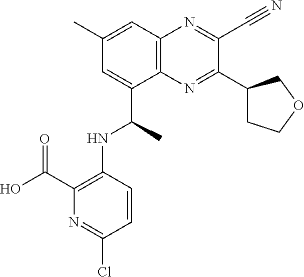 [SMILES: Cc1cc([C@@H](C)Nc2ccc(Cl)nc2C(=O)O)c2nc([C@@H]3CCOC3)c(C#N)nc2c1] | |
| | 6-chloro-3-(((R)-1-(2-cyano-7-methyl-3-((S)- | |
| | tetrahydrofuran-3-yl)quinoxalin-5- | |
| | yl)ethyl)amino)picolinic acid | |
| | AND | |
| | 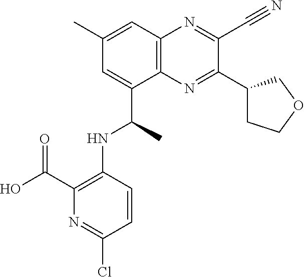 [SMILES: Cc1cc([C@@H](C)Nc2ccc(Cl)nc2C(=O)O)c2nc([C@H]3CCOC3)c(C#N)nc2c1] | |
| | 6-chloro-3-(((R)-1-(2-cyano-7-methyl-3-((R)- | |
| | tetrahydrofuran-3-yl)quinoxalin-5- | |
| | yl)ethyl)amino)picolinic acid | |
| 186 | 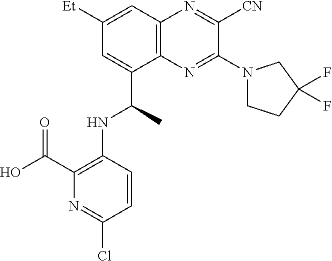 [SMILES: CCc1cc([C@@H](C)Nc2ccc(Cl)nc2C(=O)O)c2nc(N3CCC(F)(F)C3)c(C#N)nc2c1] | |
| | (R)-6-chloro-3-((1-(2-cyano-3-(3,3- | |
| | difluoropyrrolidin-1-yl)-7-ethylquinoxalin-5- | |
| | yl)ethyl)amino)picolinic acid | |
| 187 | 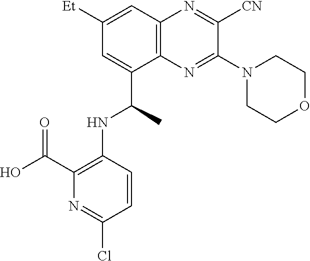 [SMILES: CCc1cc([C@@H](C)Nc2ccc(Cl)nc2C(=O)O)c2nc(N3CCOCC3)c(C#N)nc2c1] | |
| | (R)-6-chloro-3-((1-(2-cyano-7-ethyl-3- | |
| | morpholinoquinoxalin-5-yl)ethyl)amino)picolinic | |
| | acid | |
| 188 | 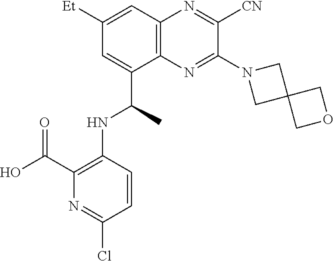 [SMILES: CCc1cc([C@@H](C)Nc2ccc(Cl)nc2C(=O)O)c2nc(N3CC4(COC4)C3)c(C#N)nc2c1] | |
| | (R)-6-chloro-3-((1-(2-cyano-7-ethyl-3-(2-oxa-6- | |
| | azaspiro[3.3]heptan-6-yl)quinoxalin-5- | |
| | yl)ethyl)amino)picolinic acid | |
| 189A and 189B | 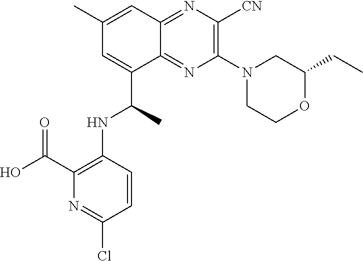 [SMILES: CC[C@H]1CN(c2nc3c([C@@H](C)Nc4ccc(Cl)nc4C(=O)O)cc(C)cc3nc2C#N)CCO1] | |
| | 6-chloro-3-(((R)-1-(2-cyano-3-((S)-2- | |
| | ethylmorpholino)-7-methylquinoxalin-5- | |
| | yl)ethyl)amino)picolinic acid | |
| | AND | |
| | 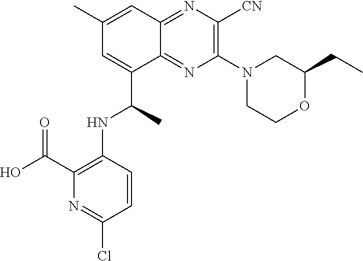 [SMILES: CC[C@@H]1CN(c2nc3c([C@@H](C)Nc4ccc(Cl)nc4C(=O)O)cc(C)cc3nc2C#N)CCO1] | |
| | 6-chloro-3-(((R)-1-(2-cyano-3-((R)-2- | |
| | ethylmorpholino)-7-methylquinoxalin-5- | |
| | yl)ethyl)amino)picolinic acid | |
| 190A | 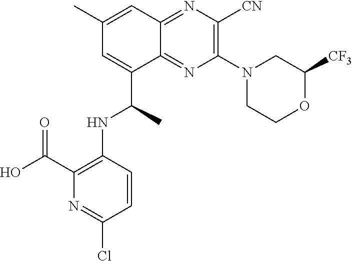 [SMILES: Cc1cc([C@@H](C)Nc2ccc(Cl)nc2C(=O)O)c2nc(N3CCO[C@H](C(F)(F)F)C3)c(C#N)nc2c1] | ¹H NMR (400 MHz, CD₃OD) δ = 7.71 (d, J = 1.6 Hz, 1H), 7.68 (s, 1H), 7.08 (d, J = 8.8 Hz, 1H), 6.86 (d, J = 9.2 Hz, 1H), 5.53 (q, J = 6.8 Hz, 1H), 4.47-4.44 (m, 1H), 4.39-4.35 (m, 1H), 4.29-4.25 (m, 1H), 4.17 (br d, J = 12.0 Hz, 1H), 3.97-3.90 (m, 1H), 3.48-3.41 (m, 1H), 3.34- 3.33 (m, 1H), 2.47 (s, 3H), 1.70 (d, J = 6.8 Hz, 3H). LCMS [M + Na]⁺ = 543.1. |
| | 6-chloro-3-(((R)-1-(2-cyano-7-methyl-3-((S)-2- | |
| | (trifluoromethyl)morpholino)quinoxalin-5- | |
| | yl)ethyl)amino)picolinic acid | |
| 190B | 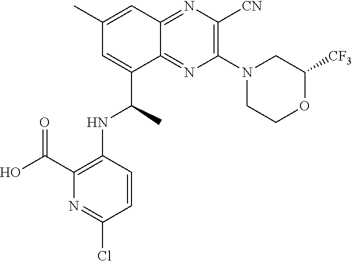 [SMILES: Cc1cc([C@@H](C)Nc2ccc(Cl)nc2C(=O)O)c2nc(N3CCO[C@@H](C(F)(F)F)C3)c(C#N)nc2c1] | ¹H NMR (400 MHz, CD₃OD) δ = 7.72 (s, 1H), 7.68 (s, 1H), 7.05 (d, J = 8.8 Hz, 1H), 6.85 (d, J = 8.8 Hz, 1H), 5.53 (q, J = 6.8 Hz, 1H), 4.44 (br d, J = 12.8 Hz, 1H), 4.39-4.34 (m, 1H), 4.27 (br d, J = 13.6 Hz, 1H), 4.19 (br d, J = 11.2 Hz, 1H), 3.95 (dt, J = 2.4, 11.6 Hz, 1H), 3.48-3.35 (m, 2H), 2.47 (s, 3H), 1.69 (d, J = 6.8 Hz, 3H). LCMS [M + Na]⁺ = 543.1. |
| | 6-chloro-3-(((R)-1-(2-cyano-7-methyl-3-((R)-2- | |
| | (trifluoromethyl)morpholino)quinoxalin-5- | |
| | yl)ethyl)amino)picolinic acid | |
| 191A and 191B | 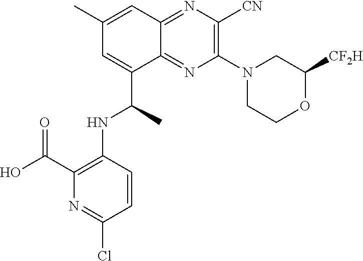 [SMILES: Cc1cc([C@@H](C)Nc2ccc(Cl)nc2C(=O)O)c2nc(N3CCO[C@H](C(F)F)C3)c(C#N)nc2c1] | |
| | 6-chloro-3-(((R)-1-(2-cyano-3-((S)-2- | |
| | (difluoromethyl)morpholino)-7-methylquinoxalin- | |
| | 5-yl)ethyl)amino)picolinic acid | |
| | AND | |
| | 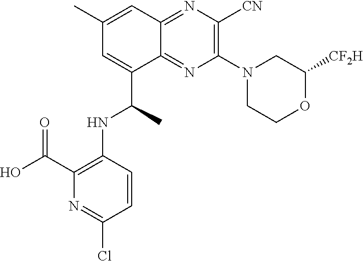 [SMILES: Cc1cc([C@@H](C)Nc2ccc(Cl)nc2C(=O)O)c2nc(N3CCO[C@@H](C(F)F)C3)c(C#N)nc2c1] | |
| | 6-chloro-3-(((R)-1-(2-cyano-3-((R)-2- | |
| | (difluoromethyl)morpholino)-7-methylquinoxalin- | |
| | 5-yl)ethyl)amino)picolinic acid | |
| 192A and 192B | 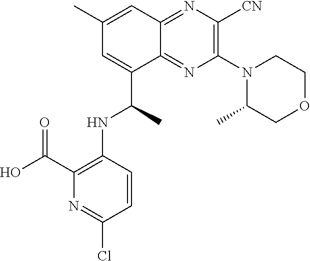 [SMILES: Cc1cc([C@@H](C)Nc2ccc(Cl)nc2C(=O)O)c2nc(N3CCOC[C@@H]3C)c(C#N)nc2c1] | |
| | 6-chloro-3-(((R)-1-(2-cyano-7-methyl-3-((S)-3- | |
| | methylmorpholino)quinoxalin-5- | |
| | yl)ethyl)amino)picolinic acid and | |
| | 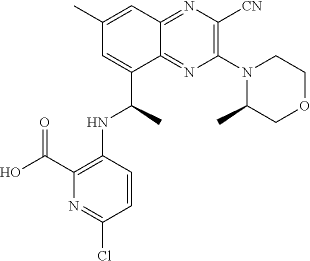 [SMILES: Cc1cc([C@@H](C)Nc2ccc(Cl)nc2C(=O)O)c2nc(N3CCOC[C@H]3C)c(C#N)nc2c1] | |
| | 6-chloro-3-(((R)-1-(2-cyano-7-methyl-3-((R)-3- | |
| | methylmorpholino)quinoxalin-5- | |
| | yl)ethyl)amino)picolinic acid | |
| 193 | 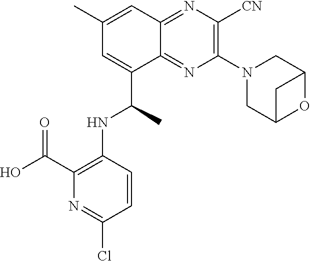 [SMILES: Cc1cc([C@@H](C)Nc2ccc(Cl)nc2C(=O)O)c2nc(N3CC4CC(C3)O4)c(C#N)nc2c1] | |
| | 3-(((1R)-1-(3-(6-oxa-3-azabicyclo[3.1.1]heptan- | |
| | 3-yl)-2-cyano-7-methylquinoxalin-5- | |
| | yl)ethyl)amino)-6-chloropicolinic acid | |
| 194A and 194B | 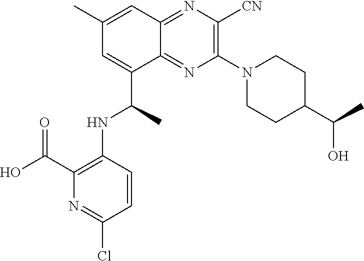 [SMILES: Cc1cc([C@@H](C)Nc2ccc(Cl)nc2C(=O)O)c2nc(N3CCC([C@@H](C)O)CC3)c(C#N)nc2c1] | |
| | 6-chloro-3-(((R)-1-(2-cyano-3-(4-((R)-1- | |
| | hydroxyethyl)piperidin-1-yl)-7-methylquinoxalin- | |
| | 5-yl)ethyl)amino)picolinic acid | |
| | And | |
| | 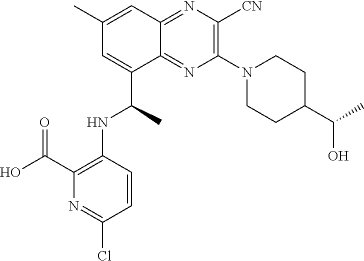 [SMILES: Cc1cc([C@@H](C)Nc2ccc(Cl)nc2C(=O)O)c2nc(N3CCC([C@H](C)O)CC3)c(C#N)nc2c1] | |
| | 6-chloro-3-(((R)-1-(2-cyano-3-(4-((S)-1- | |
| | hydroxyethyl)piperidin-1-yl)-7-methylquinoxalin- | |
| | 5-yl)ethyl)amino)picolinic acid | |
| 195A and 195B | 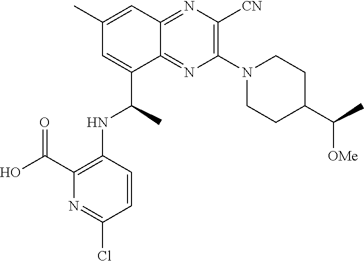 [SMILES: CO[C@H](C)C1CCN(c2nc3c([C@@H](C)Nc4ccc(Cl)nc4C(=O)O)cc(C)cc3nc2C#N)CC1] | 195A, final product from the first eluting isomer before saponification: ¹H NMR (400 MHz, methanol-d₄) δ = 7.63 (s, 2H), 7.20 (d, J = 9.2 Hz, 1H), 7.02 (d, J = 9.2 Hz, 1H), 5.55 (q, J = 6.8 Hz, 1H), 4.51-4.42 (m, 2H), 3.36 (s, 3H), 3.25- 3.18 (m, 1H), 3.17-3.05 (m, 2H), 2.45 (s, 3H), 2.05-1.97 (m, 1H), 1.84-1.78 (m, 1H), 1.72 (d, J = 6.8 Hz, 4H), 1.65- 1.48 (m, 2H), 1.17 (d, J = 6.4 Hz, 3H). LCMS [M + 1]⁺ = 509.3. 195B, final product from the second eluting isomer before |
| | 6-chloro-3-(((R)-1-(2-cyano-3-(4-((R)-1- | saponification: ¹H NMR (400 |
| | methoxyethyl)piperidin-1-yl)-7-methylquinoxalin- | MHz, methanol-d₄) δ = 7.63 (s, |
| | 5-yl)ethyl)amino)picolinic acid | 2H), 7.21 (d, J = 9.2 Hz, 1H), |
| | And | 7.03 (d, J = 9.2 Hz, 1H), 5.56 |
| | 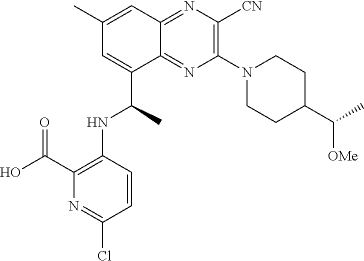 [SMILES: CO[C@@H](C)C1CCN(c2nc3c([C@@H](C)Nc4ccc(Cl)nc4C(=O)O)cc(C)cc3nc2C#N)CC1] | (q, J = 6.4 Hz, 1H), 4.51-4.41 (m, 2H), 3.36 (s, 3H), 3.23- 3.20 (m, 1H), 3.16-3.06 (m, 2H), 2.45 (s, 3H), 2.00 (br d, J = 13.2 Hz, 1H), 1.86-1.78 (m, 1H), 1.72 (d, J = 6.8 Hz, 4H), 1.65-1.48 (m, 2H), 1.17 (d, J = 6.4 Hz, 3H). LCMS [M + 1]⁺ = 509.3. |
| | 6-chloro-3-(((R)-1-(2-cyano-3-(4-((S)-1- | |
| | methoxyethyl)piperidin-1-yl)-7-methylquinoxalin- | |
| | 5-yl)ethyl)amino)picolinic acid | |
| 196A and 196B | 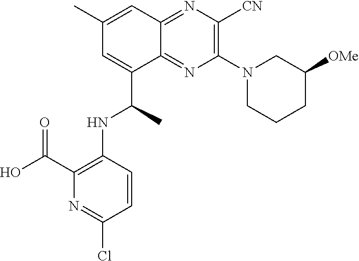 [SMILES: CO[C@H]1CCCN(c2nc3c([C@@H](C)Nc4ccc(Cl)nc4C(=O)O)cc(C)cc3nc2C#N)C1] | 196A, made from the enantiopure (3S)-3- methoxypiperidine: ¹H NMR (400 MHz, CD₃OD) δ = 7.64 (s, 2H), 7.21 (d, J = 8.8 Hz, 1H), 7.00 (d, J = 8.8 Hz, 1H), 5.57 (q, J = 6.8 Hz, 1H), 4.24 (dd, J = 3.2, 12.8 Hz, 1H), 4.00-3.89 (m, 1H), 3.65-3.55 (m, 2H), 3.47 (dd, J = 8.0, 12.8 Hz, 1H), 3.43 (s, 3H), 2.46 (s, 3H), 2.17- 1.98 (m, 2H), 1.78-1.64 (m, 5H). LCMS [M + Na]⁺ = 503.2. 196B, made from the enantiopure (3R)-3- methoxypiperidine: ¹H NMR |
| | 196A, 6-chloro-3-(((R)-1-(2-cyano-3-((S)-3- | (400 MHz, CD₃OD) δ = 7.64 (s, |
| | methoxypiperidin-1-yl)-7-methylquinoxalin-5- | 1H), 7.61 (s, 1H), 7.14 (d, J = |
| | yl)ethyl)amino)picolinic acid | 8.8 Hz, 1H), 6.97 (d, J = 9.2 Hz, |
| | And | 1H), 5.56 (q, J = 6.8 Hz, 1H), |
| | 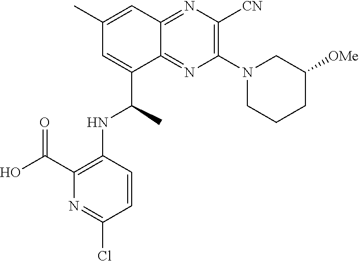 [SMILES: CO[C@@H]1CCCN(c2nc3c([C@@H](C)Nc4ccc(Cl)nc4C(=O)O)cc(C)cc3nc2C#N)C1] | 4.20-4.18 (m, 1H), 3.94-3.90 (m, 1H), 3.61-3.49 (m, 3H), 3.40 (s, 3H), 2.44 (s, 3H), 2.10- 2.02 (m, 2H), 1.70 (d, J = 6.8 Hz, 5H). LCMS [M + Na]⁺ = 503.2. |
| | 196B, 6-chloro-3-(((R)-1-(2-cyano-3-((R)-3- | |
| | methoxypiperidin-1-yl)-7-methylquinoxalin-5- | |
| | yl)ethyl)amino)picolinic acid | |
| 197 | 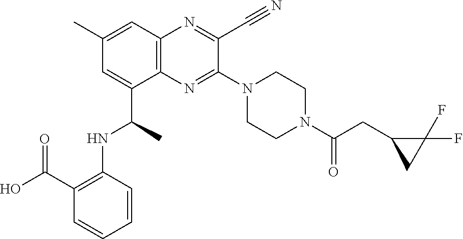 [SMILES: Cc1cc([C@@H](C)Nc2ccccc2C(=O)O)c2nc(N3CCN(C(=O)C[C@@H]4CC4(F)F)CC3)c(C#N)nc2c1] | Product generated using the first eluting isomer before saponification: ¹H NMR (400 MHz, CD₃OD) δ = 7.90 (d, J = 7.6 Hz 1H), 7.69 (s, 1H) 7.65 (s, 1H), 7.12 (t, J = 8.0 Hz, 1H), 6.50 (t, J = 7.6 Hz, 1H), 6.42 (d, J = 8.4 Hz, 1H), 5.60 (q, J = 6.8 Hz, 1H), 3.89-3.81 (m, 8H), 2.71 (br d, J = 7.2 Hz, 2H), 2.47 (s, 3H), 1.96-1.93 (m, 1H), 1.69 (d, J = 6.8 Hz, 3H), 1.59- 1.55 (m, 1H), 1.17-1.13 (m, 1H). LCMS [M + Na]⁺ = 557.2. |
| | 2-(((R)-1-(2-cyano-3-(4-(2-((R)-2,2- | |
| | difluorocyclopropyl)acetyl)piperazin-1-yl)-7- | |
| | methylquinoxalin-5-yl)ethyl)amino)benzoic acid | |
| | OR | |
| | 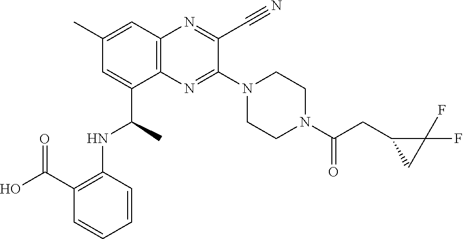 [SMILES: Cc1cc([C@@H](C)Nc2ccccc2C(=O)O)c2nc(N3CCN(C(=O)C[C@H]4CC4(F)F)CC3)c(C#N)nc2c1] | |
| | 2-(((R)-1-(2-cyano-3-(4-(2-((S)-2,2- | |
| | difluorocyclopropyl)acetyl)piperazin-1-yl)-7- | |
| | methylquinoxalin-5-yl)ethyl)amino)benzoic acid | |
| 198 | 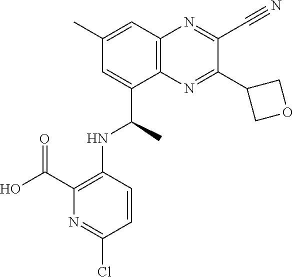 [SMILES: Cc1cc([C@@H](C)Nc2ccc(Cl)nc2C(=O)O)c2nc(C3COC3)c(C#N)nc2c1] | |
| | (R)-6-chloro-3-((1-(2-cyano-7-methyl-3-(oxetan- | |
| | 3-yl)quinoxalin-5-yl)ethyl)amino)picolinic acid | |
| 199 | 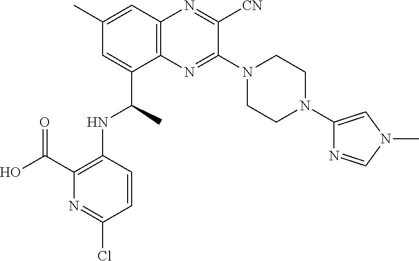 [SMILES: Cc1cc([C@@H](C)Nc2ccc(Cl)nc2C(=O)O)c2nc(N3CCN(c4cn(C)cn4)CC3)c(C#N)nc2c1] | |
| | (R)-6-chloro-3-((1-(2-cyano-7-methyl-3-(4-(1- | |
| | methyl-1H-imidazol-4-yl)piperazin-1- | |
| | yl)quinoxalin-5-yl)ethyl)amino)picolinic acid | |
| 200 | 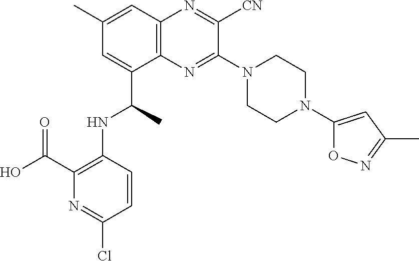 [SMILES: Cc1cc([C@@H](C)Nc2ccc(Cl)nc2C(=O)O)c2nc(N3CCN(c4cc(C)no4)CC3)c(C#N)nc2c1] | LCMS [M + H]⁺ = 533.3 |
| | (R)-6-chloro-3-((1-(2-cyano-7-methyl-3-(4-(3- | |
| | methylisoxazol-5-yl)piperazin-1-yl)quinoxalin-5- | |
| | yl)ethyl)amino)picolinic acid | |
| 201 | 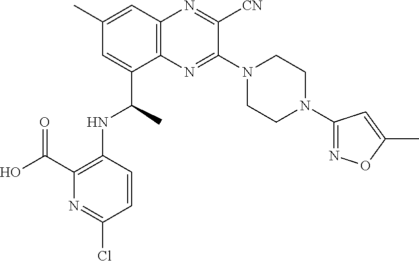 [SMILES: Cc1cc([C@@H](C)Nc2ccc(Cl)nc2C(=O)O)c2nc(N3CCN(c4cc(C)on4)CC3)c(C#N)nc2c1] | ¹H NMR (400 MHz, CDCl₃) δ = 8.55-8.33 (m, 1H), 7.67 (s, 1H), 7.56 (s, 1H), 7.14-7.03 (m, 1H), 6.75 (br d, J = 8.8 Hz, 1H), 5.71 (s, 1H), 5.54-5.44 (m, 1H), 3.92-3.84 (m, 4H), 3.57-3.50 (m, 4H), 2.47 (s, 3H), 2.35 (s, 3H), 1.70 (br d, J = 6.4 Hz, 3H). LCMS [M + Na]⁺ = 555.2. |
| | (R)-6-chloro-3-((1-(2-cyano-7-methyl-3-(4-(5- | |
| | methylisoxazol-3-yl)piperazin-1-yl)quinoxalin-5- | |
| | yl)ethyl)amino)picolinic acid | |
| 202 | 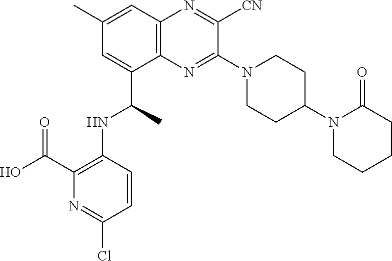 [SMILES: Cc1cc([C@@H](C)Nc2ccc(Cl)nc2C(=O)O)c2nc(N3CCC(N4CCCCC4=O)CC3)c(C#N)nc2c1] | ¹H NMR (400 MHz, CDCl₃) δ = 8.34 (br d, J = 6.0 Hz, 1H), 7.65 (s, 1H), 7.54 (d, J = 1.6 Hz, 1H), 7.12 (d, J = 9.2 Hz, 1H), 6.79 (d, J = 8.8 Hz, 1H), 5.52 (quin, J = 6.8 Hz, 1H), 4.94-4.80 (m, 1H), 4.52-4.39 (m, 2H), 3.29- 3.15 (m, 4H), 2.51-2.41 (m, 5H), 2.05-1.94 (m, 2H), 1.90- 1.79 (m, 6H), 1.70 (d, J = 6.4 Hz, 3H). LCMS [M + 1]⁺ = 548.3. |
| | (R)-6-chloro-3-((1-(2-cyano-7-methyl-3-(2-oxo- | |
| | [1,4′-bipiperidin]-1′-yl)quinoxalin-5- | |
| | yl)ethyl)amino)picolinic acid | |
| 203 | 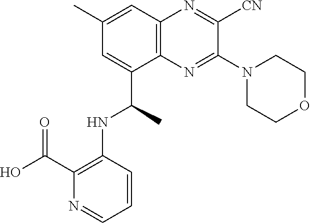 [SMILES: Cc1cc([C@@H](C)Nc2cccnc2C(=O)O)c2nc(N3CCOCC3)c(C#N)nc2c1] | ¹H NMR (400 MHz, methanol- d₄) δ = 7.73 (dd, J = 0.8, 4.8 Hz, 1H), 7.71 (d, J = 2.0 Hz, 1H), 7.66 (s, 1H), 7.38 (dd, J = 4.8, 8.8 Hz, 1H), 7.21 (d, J = 8.4 Hz, 1H), 5.61 (q, J = 6.8 Hz, 1H), 3.95-3.89 (m, 4H), 3.82-3.76 (m, 4H), 2.46 (s, 3H), 1.73 (d, J = 6.4 Hz, 3H). LCMS [M + 1]⁺ = 419.2. |
| | (R)-3-((1-(2-cyano-7-methyl-3- | |
| | morpholinoquinoxalin-5-yl)ethyl)amino)picolinic | |
| | acid | |
| 204 | 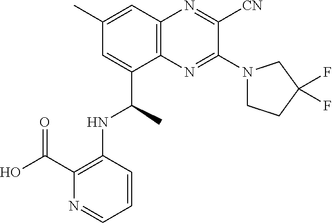 [SMILES: Cc1cc([C@@H](C)Nc2cccnc2C(=O)O)c2nc(N3CCC(F)(F)C3)c(C#N)nc2c1] | ¹H NMR (400 MHz, methanol- d4) δ = 7.78-7.70 (m, 1H), 7.65 (d, J = 1.6 Hz, 1H), 7.59 (s, 1H), 7.48 (dd, J = 5.2, 8.8 Hz, 1H), 7.34 (d, J = 8.8 Hz, 1H), 5.58 (q, J = 6.8 Hz, 1H), 4.40-4.14 (m, 4H), 2.66-2.53 (m, 2H), 2.43 (s, 3H), 1.75 (d, J = 6.8 Hz, 3H). LCMS [M + Na]⁺ = 461.2. |
| | (R)-3-((1-(2-cyano-3-(3,3-difluoropyrrolidin-1-yl)- | |
| | 7-methylquinoxalin-5-yl)ethyl)amino)picolinic | |
| | acid | |
| 205 | 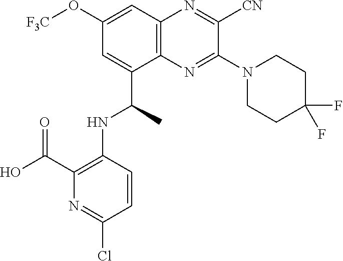 [SMILES: C[C@@H](Nc1ccc(Cl)nc1C(=O)O)c1cc(OC(F)(F)F)cc2nc(C#N)c(N3CCC(F)(F)CC3)nc12] | |
| | (R)-6-chloro-3-((1-(2-cyano-3-(4,4- | |
| | difluoropiperidin-1-yl)-7- | |
| | (trifluoromethoxy)quinoxalin-5- | |
| | yl)ethyl)amino)picolinic acid | |
| 206 | 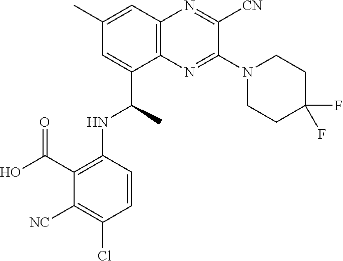 [SMILES: Cc1cc([C@@H](C)Nc2ccc(Cl)c(C#N)c2C(=O)O)c2nc(N3CCC(F)(F)CC3)c(C#N)nc2c1] | |
| | (R)-3-chloro-2-cyano-6-((1-(2-cyano-3-(4,4- | |
| | difluoropiperidin-1-yl)-7-methylquinoxalin-5- | |
| | yl)ethyl)amino)benzoic acid | |
| 207 | 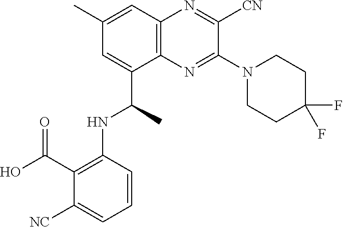 [SMILES: Cc1cc([C@@H](C)Nc2cccc(C#N)c2C(=O)O)c2nc(N3CCC(F)(F)CC3)c(C#N)nc2c1] | |
| | (R)-2-cyano-6-((1-(2-cyano-3-(4,4- | |
| | difluoropiperidin-1-yl)-7-methylquinoxalin-5- | |
| | yl)ethyl)amino)benzoic acid | |
| 208 | 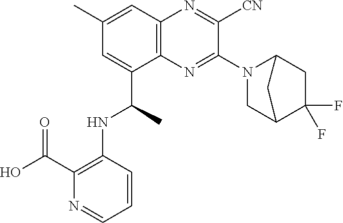 [SMILES: Cc1cc([C@@H](C)Nc2cccnc2C(=O)O)c2nc(N3CC4CC3CC4(F)F)c(C#N)nc2c1] | |
| | 3-(((1R)-1-(2-cyano-3-(5,5-difluoro-2- | |
| | azabicyclo[2.2.1]heptan-2-yl)-7- | |
| | methylquinoxalin-5-yl)ethyl)amino)picolinic acid | |
| 209 | 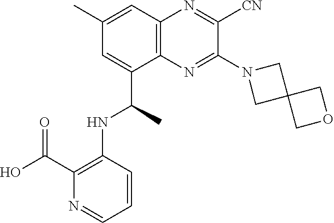 [SMILES: Cc1cc([C@@H](C)Nc2cccnc2C(=O)O)c2nc(N3CC4(COC4)C3)c(C#N)nc2c1] | |
| | (R)-3-((1-(2-cyano-7-methyl-3-(2-oxa-6- | |
| | azaspiro[3.3]heptan-6-yl)quinoxalin-5- | |
| | yl)ethyl)amino)picolinic acid | |
| 210A and 210B | 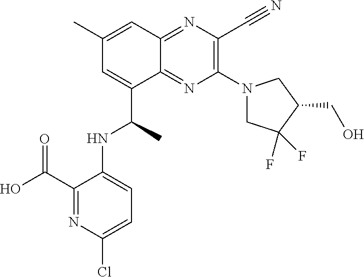 [SMILES: Cc1cc([C@@H](C)Nc2ccc(Cl)nc2C(=O)O)c2nc(N3C[C@H](CO)C(F)(F)C3)c(C#N)nc2c1] | 210A, final product formed using the first eluting isomer before saponification: ¹H NMR (400 MHz, CD₃OD) δ = 7.61- 7.58 (m, 2H), 7.18 (d, J = 9.2 Hz, 1H), 7.02 (d, J = 8.8 Hz, 1H), 5.52 (q, J = 6.8 Hz, 1H), 4.43-4.29 (m, 3H), 4.02 (dd, J = 6.8, 10.8 Hz, 1H), 3.94 (dd, J = 5.2, 11.2 Hz, 1H), 3.80 (dd, J = 6.8, 11.6 Hz, 1H), 2.99-2.87 (m, 1H), 2.43 (s, 3H), 1.72 (d, J = 6.4 Hz, 3H). LCMS [M + 1]⁺ = 503.2. 210B, final product formed using the second eluting isomer before saponification: |
| | 6-chloro-3-(((R)-1-(2-cyano-3-((R)-3,3-difluoro-4- | ¹H NMR (400 MHz, CD₃OD) |
| | (hydroxymethyl)pyrrolidin-1-yl)-7- | δ = 7.61-7.58 (m, 2H), 7.19 (d, |
| | methylquinoxalin-5-yl)ethyl)amino)picolinic acid | J = 8.8 Hz, 1H), 7.03 (d, J = 9.2 |
| | AND | Hz, 1H), 5.51 (q, J = 6.4 Hz, |
| | 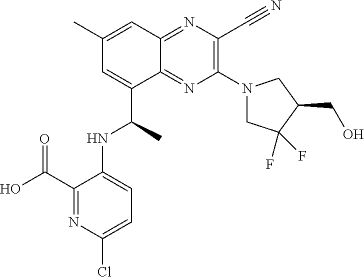 [SMILES: Cc1cc([C@@H](C)Nc2ccc(Cl)nc2C(=O)O)c2nc(N3C[C@@H](CO)C(F)(F)C3)c(C#N)nc2c1] | 1H), 4.40-4.28 (m, 3H), 4.07 (dd, J = 6.8, 10.8 Hz, 1H), 3.94 (dd, J = 5.2, 11.4 Hz, 1H), 3.82 (dd, J = 6.8, 11.4 Hz, 1H), 2.99- 2.86 (m, 1H), 2.44 (s, 3H), 1.73 (d, J = 6.8 Hz, 3H). LCMS [M + 1]⁺ = 503.2. |
| | 6-chloro-3-(((R)-1-(2-cyano-3-((S)-3,3-difluoro-4- | |
| | (hydroxymethyl)pyrrolidin-1-yl)-7- | |
| | methylquinoxalin-5-yl)ethyl)amino)picolinic acid | |
| 211A and 211B | 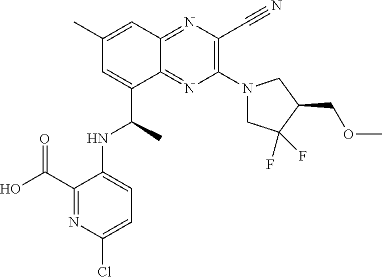 [SMILES: COC[C@@H]1CN(c2nc3c([C@@H](C)Nc4ccc(Cl)nc4C(=O)O)cc(C)cc3nc2C#N)CC1(F)F] | 211A, final product was synthesized using the first eluting isomer from SFC directly before saponification: 1H NMR (400 MHz, CD₃OD) δ = 7.61 (s, 1H), 7.58 (s, 1H), 7.19 (d, J = 9.2 Hz, 1H), 7.03 (d, J = 9.2 Hz, 1H), 5.51 (q, J = 6.8 Hz, 1H), 4.42-4.27 (m, 3H), 3.95 (dd, J = 7.6, 10.8 Hz, 1H), 3.78-3.72 (m, 1H), 3.63 (dd, J = 7.2, 9.6 Hz, 1H), 3.40 (s, 3H), 3.12-2.98 (m, 1H), 2.43 (s, 3H), 1.72 (d, J = 6.4 Hz, 3H). LCMS [M + Na]⁺ = 539.1. 211B, final product was |
| | 6-chloro-3-(((R)-1-(2-cyano-3-((S)-3,3-difluoro-4- | synthesized using the second |
| | (methoxymethyl)pyrrolidin-1-yl)-7- | eluting isomer from SFC |
| | methylquinoxalin-5-yl)ethyl)amino)picolinic acid | directly before saponification: |
| | AND | 1H NMR (400 MHz, CD₃OD) δ = |
| | 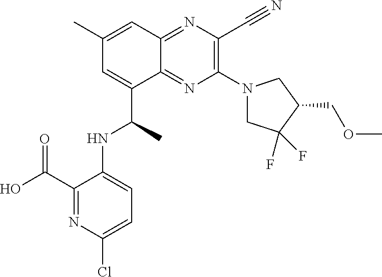 [SMILES: COC[C@H]1CN(c2nc3c([C@@H](C)Nc4ccc(Cl)nc4C(=O)O)cc(C)cc3nc2C#N)CC1(F)F] | ¹H NMR (400 MHz, methanol- d₄) δ = 7.61 (s, 1H), 7.57 (br s, 1H), 7.19 (d, J = 8.8 Hz, 1H), 7.03 (d, J = 9.2 Hz, 1H), 5.50 (q, J = 6.4 Hz, 1H), 4.42-4.22 (m, 3H), 4.01 (br dd, J = 7.2, 10.8 Hz, 1H), 3.76 (dd, J = 5.6, 9.6 Hz, 1H), 3.64 (dd, J = 6.8, 9.6 Hz, 1H), 3.40 (s, 3H), 3.10- 2.97 (m, 1H), 2.43 (s, 3H), 1.73 (d, J = 6.8 Hz, 3H). LCMS [M + Na]⁺ = 539.1. |
| | 6-chloro-3-(((R)-1-(2-cyano-3-((R)-3,3-difluoro-4- | |
| | (methoxymethyl)pyrrolidin-1-yl)-7- | |
| | methylquinoxalin-5-yl)ethyl)amino)picolinic acid | |
| 212A and 212B | 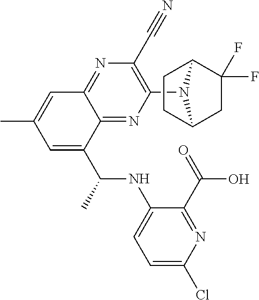 [SMILES: Cc1cc([C@@H](C)Nc2ccc(Cl)nc2C(=O)O)c2nc(N3[C@@H]4CC[C@H]3C(F)(F)C4)c(C#N)nc2c1] | |
| | 6-chloro-3-(((R)-1-(2-cyano-3-((1S,4R)-2,2-difluoro-7- | |
| | azabicyclo[2.2.1]heptan-7-yl)-7-methylquinoxalin-5- | |
| | yl)ethyl)amino)picolinic acid | |
| | AND | |
| | 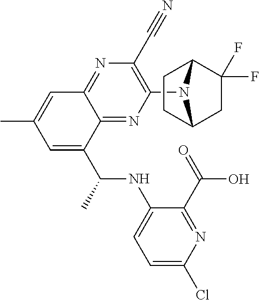 [SMILES: Cc1cc([C@@H](C)Nc2ccc(Cl)nc2C(=O)O)c2nc(N3[C@H]4CC[C@@H]3C(F)(F)C4)c(C#N)nc2c1] | |
| | 6-chloro-3-(((R)-1-(2-cyano-3-((1R,4S)-2,2-difluoro-7- | |
| | azabicyclo[2.2.1]heptan-7-yl)-7-methylquinoxalin-5- | |
| | yl)ethyl)amino)picolinic acid | |
| 213 | 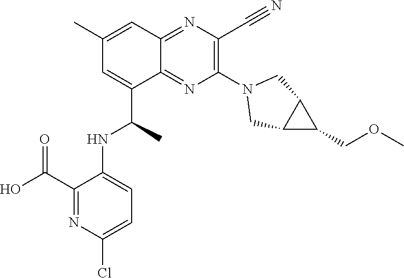 [SMILES: COC[C@H]1[C@H]2CN(c3nc4c([C@@H](C)Nc5ccc(Cl)nc5C(=O)O)cc(C)cc4nc3C#N)C[C@@H]12] | ¹H NMR (400 MHz, CD₃OD) δ = 7.59 (dd, J = 1.2, 8.8 Hz, 2H), 7.22 (d, J = 8.8 Hz, 1H), 7.05 (d, J = 8.8 Hz, 1H), 5.53 (q, J = 6.8 Hz, 1H), 4.22-4.08 (m, 4H), 3.47-3.44 (m, 2H), 3.32 (s, 3H), 2.43 (s, 3H), 2.07-2.04 (m, 2H), 1.74 (d, J = 6.4 Hz, 3H), 1.40 (q, J = 8.0 Hz, 1H). LCMS [M + 1]⁺ = 493.1. |
| | 6-chloro-3-(((R)-1-(2-cyano-3-((1R,5S,6S)-6- | |
| | (methoxymethyl)-3-azabicyclo[3.1.0]hexan-3-yl)-7- | |
| | methylquinoxalin-5-yl)ethyl)amino)picolinic acid | |
| 214 | 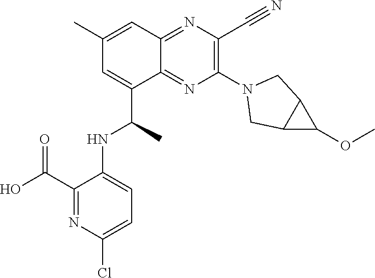 [SMILES: COC1C2CN(c3nc4c([C@@H](C)Nc5ccc(Cl)nc5C(=O)O)cc(C)cc4nc3C#N)CC21] | |
| | 6-chloro-3-(((1R)-1-(2-cyano-3-(6-methoxy-3- | |
| | azabicyclo[3.1.0]hexan-3-yl)-7-methylquinoxalin-5- | |
| | yl)ethyl)amino)picolinic acid | |
| 215 | 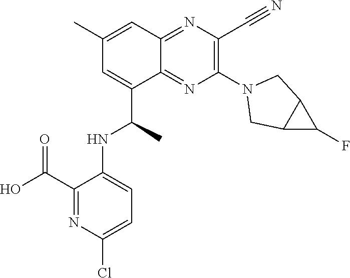 [SMILES: Cc1cc([C@@H](C)Nc2ccc(Cl)nc2C(=O)O)c2nc(N3CC4C(F)C4C3)c(C#N)nc2c1] | ¹H NMR (400 MHz, CD₃OD) δ = 7.57 (s, 1H), 7.53 (s, 1H), 7.19 (d, J = 8.8 Hz, 1H), 7.00 (d, J = 9.2 Hz, 1H), 5.47 (q, J = 6.8 Hz, 1H), 4.55-4.38 (m, 1H), 4.33 (t, J = 11.6 Hz, 2H), 4.03-3.93 (m, 2H), 2.41 (s, 3H), 2.27 (d, J = 3.2 Hz, 1H), 2.23 (d, J = 3.2 Hz, 1H), 1.71 (d, J = 6.4 Hz, 3H). LCMS [M + 23]⁺ = 489.1. |
| | 6-chloro-3-(((1R)-1-(2-cyano-3-(6-fluoro-3- | |
| | azabicyclo[3.1.0]hexan-3-yl)-7-methylquinoxalin- | |
| | 5-yl)ethyl)amino)picolinic acid | |
| 216 | 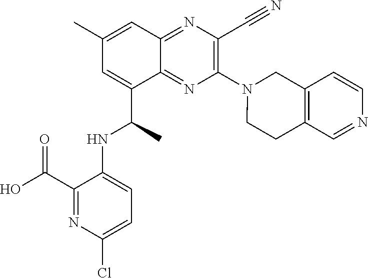 [SMILES: Cc1cc([C@@H](C)Nc2ccc(Cl)nc2C(=O)O)c2nc(N3CCc4cnccc4C3)c(C#N)nc2c1] | ¹H NMR (400 MHz, CD₃OD) δ = 8.43 (br s, 1H), 8.36 (br d, J = 1.2 Hz, 1H), 7.67 (d, J = 4.0 Hz, 2H), 7.44 (br d, J = 4.8 Hz, 1H), 7.17 (d, J = 8.8 Hz, 1H), 7.01 (d, J = 8.8 Hz, 1H), 5.51 (q, J = 6.4 Hz, 1H), 5.03-4.92 (m, 2H), 4.16 (br t, J = 5.2 Hz, 2H), 3.21-3.14 (m, 2H), 2.47 (s, 3H), 1.72 (d, J = 6.8 Hz, 3H). LCMS [M + 1]⁺: 500.1. |
| | (R)-6-chloro-3-((1-(2-cyano-3-(3,4-dihydro-2,6- | |
| | naphthyridin-2(1H)-yl)-7-methylquinoxalin-5- | |
| | yl)ethyl)amino)picolinic acid | |
| 217 | 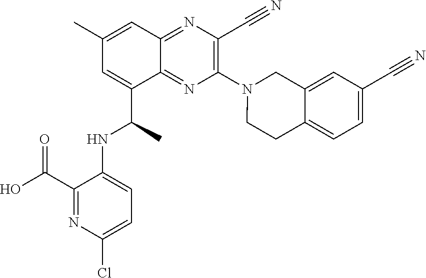 [SMILES: Cc1cc([C@@H](C)Nc2ccc(Cl)nc2C(=O)O)c2nc(N3CCc4ccc(C#N)cc4C3)c(C#N)nc2c1] | ¹H NMR (400 MHz, CD₃OD) δ = 7.71 (s, 1H), 7.66 (d, J = 2.0 Hz, 1H), 7.64 (d, J = 0.8 Hz, 1H), 7.54 (dd, J = 1.6, 8.0 Hz, 1H), 7.40 (d, J = 8.0 Hz, 1H), 7.17 (d, J = 9.2 Hz, 1H), 7.03 (d, J = 8.8 Hz, 1H), 5.53 (q, J = 6.8 Hz, 1H), 5.01-4.93 (m, 2H), 4.12 (t, J = 6.0 Hz, 2H), 3.20 (br t, J = 5.6 Hz, 2H), 2.46 (s, 3H), 1.71 (d, J = 6.8 Hz, 3H). LCMS [M + 23]⁺ = 546.2. |
| | (R)-6-chloro-3-((1-(2-cyano-3-(7-cyano-3,4- | |
| | dihydroisoquinolin-2(1H)-yl)-7-methylquinoxalin-5- | |
| | yl)ethyl)amino)picolinic acid | |
| 218 | 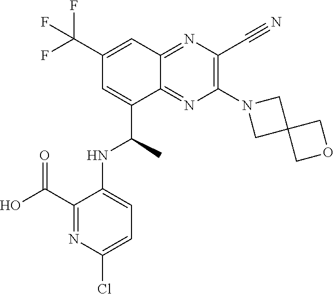 [SMILES: C[C@@H](Nc1ccc(Cl)nc1C(=O)O)c1cc(C(F)(F)F)cc2nc(C#N)c(N3CC4(COC4)C3)nc12] | |
| | (R)-6-chloro-3-((1-(2-cyano-3-(2-oxa-6- | |
| | azaspiro[3.3]heptan-6-yl)-7- | |
| | (trifluoromethyl)quinoxalin-5- | |
| | yl)ethyl)amino)picolinic acid | |
| 219 | 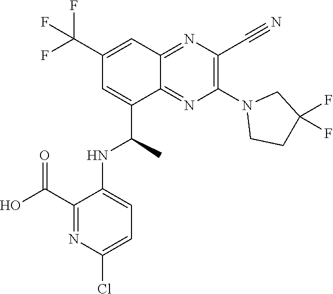 [SMILES: C[C@@H](Nc1ccc(Cl)nc1C(=O)O)c1cc(C(F)(F)F)cc2nc(C#N)c(N3CCC(F)(F)C3)nc12] | |
| | (R)-6-chloro-3-((1-(2-cyano-3-(3,3-difluoropyrrolidin- | |
| | 1-yl)-7-(trifluoromethyl)quinoxalin-5- | |
| | yl)ethyl)amino)picolinic acid | |
| 220 | 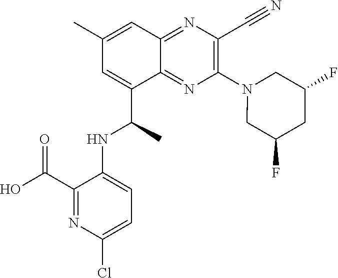 [SMILES: Cc1cc([C@@H](C)Nc2ccc(Cl)nc2C(=O)O)c2nc(N3C[C@H](F)C[C@@H](F)C3)c(C#N)nc2c1] | |
| | 6-chloro-3-(((R)-1-(2-cyano-3-((3R,5R)-3,5- | |
| | difluoropiperidin-1-yl)-7-methylquinoxalin-5- | |
| | yl)ethyl)amino)picolinic acid | |
| 221 | 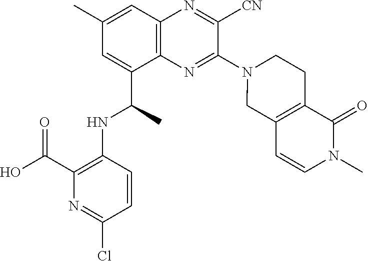 [SMILES: Cc1cc([C@@H](C)Nc2ccc(Cl)nc2C(=O)O)c2nc(N3CCc4c(ccn(C)c4=O)C3)c(C#N)nc2c1] | ¹H NMR (400 MHz, CD₃OD) δ = 7.67 (br d, J = 4.0 Hz, 2H), 7.51 (d, J = 6.8 Hz, 1H), 7.15 (d, J = 9.2 Hz, 1H), 6.97 (d, J = 9.2 Hz, 1H), 6.40 (d, J = 7.2 Hz, 1H), 5.52 (q, J = 6.4 Hz, 1H), 4.74 (s, 2H), 4.16-4.02 (m, 2H), 3.56 (s, 3H), 2.93-2.76 (m, 2H), 2.47 (s, 3H), 1.71 (d, J = 6.8 Hz, 3H). LCMS [M + 1]⁺: 530.3. |
| | (R)-6-chloro-3-((1-(2-cyano-7-methyl-3-(6- | |
| | methyl-5-oxo-3,4,5,6-tetrahydro-2,6- | |
| | naphthyridin-2(1H)-yl)quinoxalin-5- | |
| | yl)ethyl)amino)picolinic acid | |
| 222 | 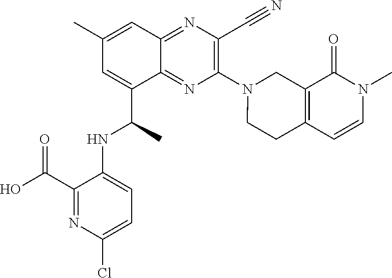 [SMILES: Cc1cc([C@@H](C)Nc2ccc(Cl)nc2C(=O)O)c2nc(N3CCc4ccn(C)c(=O)c4C3)c(C#N)nc2c1] | ¹H NMR (400 MHz, CD₃OD) δ = 7.65 (d, J = 4.4 Hz, 2H), 7.51 (d, J = 6.8 Hz, 1H), 7.17 (d, J = 8.8 Hz, 1H), 7.00 (d, J = 8.8 Hz, 1H), 6.31 (d, J = 6.8 Hz, 1H), 5.70 (q, J = 6.4 Hz, 1H), 4.68 (br d, J = 4.8 Hz, 2H), 4.10- 4.04 (m, 2H), 3.56 (s, 3H), 3.00 (br t, J = 5.4 Hz, 2H), 2.46 (s, 3H), 1.69 (d, J = 6.4 Hz, 3H). LCMS [M + H]⁺ = 530.3. |
| | (R)-6-chloro-3-((1-(2-cyano-7-methyl-3-(7-methyl-8- | |
| | oxo-3,4,7,8-tetrahydro-2,7-naphthyridin-2(1H)- | |
| | yl)quinoxalin-5-yl)ethyl)amino)picolinic acid | |
| 223 | 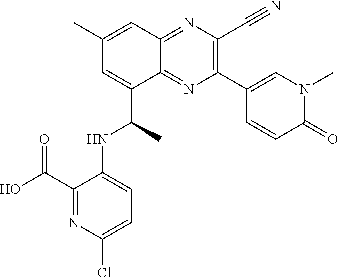 [SMILES: Cc1cc([C@@H](C)Nc2ccc(Cl)nc2C(=O)O)c2nc(-c3ccc(=O)n(C)c3)c(C#N)nc2c1] | ¹H NMR (400 MHz, CD₃OD) δ = 8.64 (d, J = 2.4 Hz, 1H), 8.32 (dd, J = 2.4, 9.4 Hz, 1H), 7.86 (s, 1H), 7.84 (s, 1H), 7.16-7.14 (m, 1H), 7.10-7.06 (m, 1H), 6.75 (d, J = 9.6 Hz, 1H), 5.74- 5.68 (m, 1H), 3.76 (s, 3H), 2.57 (s, 3H), 1.78 (d, J = 6.8 Hz, 3H). LCMS [M + 1]⁺ = 475.2. |
| | (R)-6-chloro-3-((1-(2-cyano-7-methyl-3-(1-methyl-6- | |
| | oxo-1,6-dihydropyridin-3-yl)quinoxalin-5- | |
| | yl)ethyl)amino)picolinic acid | |
| 224 | 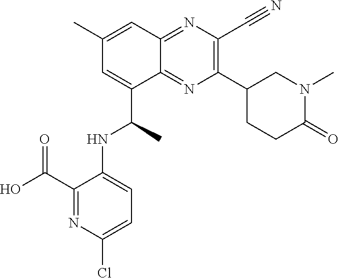 [SMILES: Cc1cc([C@@H](C)Nc2ccc(Cl)nc2C(=O)O)c2nc(C3CCC(=O)N(C)C3)c(C#N)nc2c1] | |
| | 6-chloro-3-(((1R)-1-(2-cyano-7-methyl-3-(1-methyl- | |
| | 6-oxopiperidin-3-yl)quinoxalin-5- | |
| | yl)ethyl)amino)picolinic acid | |
| 225 | 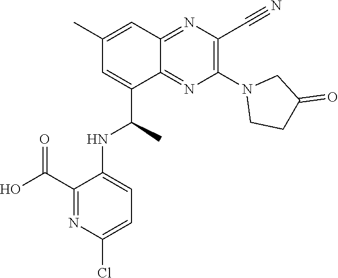 [SMILES: Cc1cc([C@@H](C)Nc2ccc(Cl)nc2C(=O)O)c2nc(N3CCC(=O)C3)c(C#N)nc2c1] | |
| | (R)-6-chloro-3-((1-(2-cyano-7-methyl-3-(3- | |
| | oxopyrrolidin-1-yl)quinoxalin-5- | |
| | yl)ethyl)amino)picolinic acid | |
| 226 | 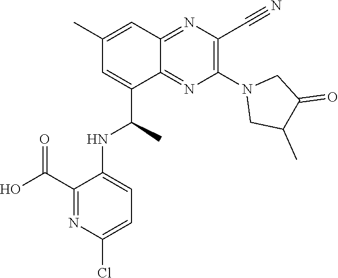 [SMILES: Cc1cc([C@@H](C)Nc2ccc(Cl)nc2C(=O)O)c2nc(N3CC(=O)C(C)C3)c(C#N)nc2c1] | |
| | 6-chloro-3-(((1R)-1-(2-cyano-7-methyl-3-(3-methyl- | |
| | 4-oxopyrrolidin-1-yl)quinoxalin-5- | |
| | yl)ethyl)amino)picolinic acid | |
| 227 | 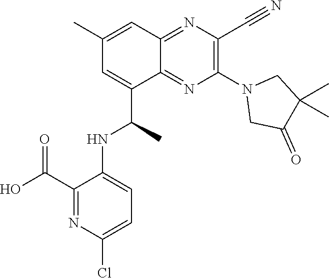 [SMILES: Cc1cc([C@@H](C)Nc2ccc(Cl)nc2C(=O)O)c2nc(N3CC(=O)C(C)(C)C3)c(C#N)nc2c1] | |
| | (R)-6-chloro-3-((1-(2-cyano-3-(3,3-dimethyl-4- | |
| | oxopyrrolidin-1-yl)-7-methylquinoxalin-5- | |
| | yl)ethyl)amino)picolinic acid | |
| 228 | 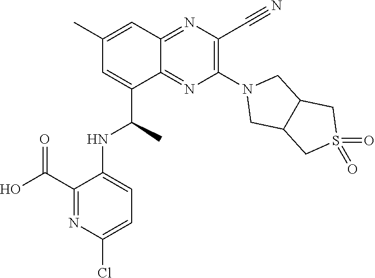 [SMILES: Cc1cc([C@@H](C)Nc2ccc(Cl)nc2C(=O)O)c2nc(N3CC4CS(=O)(=O)CC4C3)c(C#N)nc2c1] | ¹H NMR (400 MHz, CD₃OD) δ = 7.60 (d, J = 2.0 Hz, 1H), 7.58 (d, J = 0.8 Hz, 1H), 7.20 (d, J = 9.2 Hz, 1H), 7.05 (d, J = 9.2 Hz, 1H), 5.50 (q, J = 6.8 Hz, 1H), 4.38-4.25 (m, 2H), 4.09 (dd, J = 4.4, 11.6 Hz, 1H), 4.03 (dd, J = 4.8, 11.6 Hz, 1H), 3.51-3.35 (m, 4H), 3.29-3.19 (m, 2H), 2.44 (s, 3H), 1.73 (d, J = 6.4 Hz, 3H). LCMS [M + Na]⁺: 549.2. |
| | 6-chloro-3-(((1R)-1-(2-cyano-3-(2,2- | |
| | dioxidotetrahydro-1H-thieno[3,4-c]pyrrol-5(3H)-yl)- | |
| | 7-methylquinoxalin-5-yl)ethyl)amino)picolinic acid | |
| 229A and 229B | 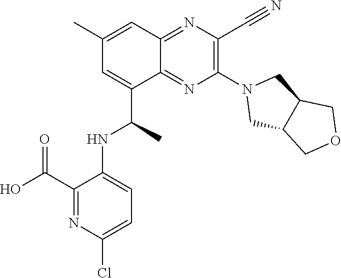 [SMILES: Cc1cc([C@@H](C)Nc2ccc(Cl)nc2C(=O)O)c2nc(N3C[C@H]4COC[C@@H]4C3)c(C#N)nc2c1] | 229A, final product obtained using the first eluting isomer from SFC before saponification: ¹H NMR (400 MHz, CD₃OD) δ = 7.56 (s, 1H), 7.53 (s, 1H), 7.16 (d, J = 8.8 Hz, 1H), 7.00 (d, J = 8.8 Hz, 1H), 5.49 (q, J = 6.8 Hz, 1H), 4.18 (br dd, J = 6.0, 9.6 Hz, 2H), 4.06 (t, J = 6.4 Hz, 2H), 3.73-3.52 (m, 4H), 2.77-2.59 (m, 2H), 2.41 (s, 3H), 1.70 (d, J = 6.8 Hz, 3H). LCMS [M + Na]+: 501.1. 229B, final product obtained using the second eluting isomer from SFC before saponification: ¹H NMR (400 |
| | 6-chloro-3-(((R)-1-(2-cyano-7-methyl-3-((3aS,6aS)- | MHz, CD₃OD) δ = 7.57 (d, J = |
| | tetrahydro-1H-furo[3,4-c]pyrrol-5(3H)-yl)quinoxalin- | 2.0 Hz, 1H), 7.54 (s, 1H), 7.18 |
| | 5-yl)ethyl)amino)picolinic acid | (d, J = 9.2 Hz, 1H), 7.01 (d, J = |
| | AND | 9.2 Hz, 1H), 5.45 (q, J = 6.4 Hz, |
| | 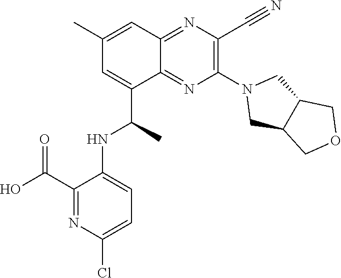 [SMILES: Cc1cc([C@@H](C)Nc2ccc(Cl)nc2C(=O)O)c2nc(N3C[C@@H]4COC[C@H]4C3)c(C#N)nc2c1] | 1H), 4.13 (dd, J = 6.0, 9.6 Hz, 2H), 4.06 (t, J = 6.8 Hz, 2H), 3.75 (br t, J = 10.4 Hz, 2H), 3.62 (dd, J = 7.2, 10.4 Hz, 2H), 2.78- 2.58 (m, 2H), 2.42 (s, 3H), 1.72 (d, J = 6.4 Hz, 3H). LCMS [M + Na]+: 501.1. |
| | 6-chloro-3-(((R)-1-(2-cyano-7-methyl-3-((3aR,6aR)- | |
| | tetrahydro-1H-furo[3,4-c]pyrrol-5(3H)-yl)quinoxalin- | |
| | 5-yl)ethyl)amino)picolinic acid | |
| 230 | 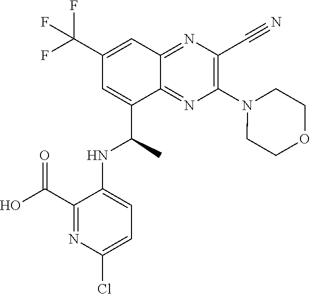 [SMILES: C[C@@H](Nc1ccc(Cl)nc1C(=O)O)c1cc(C(F)(F)F)cc2nc(C#N)c(N3CCOCC3)nc12] | |
| | (R)-6-chloro-3-((1-(2-cyano-3-morpholino-7- | |
| | (trifluoromethyl)quinoxalin-5- | |
| | yl)ethyl)amino)picolinic acid | |
| 231 | 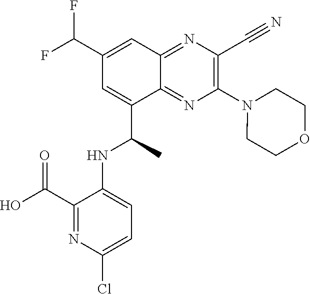 [SMILES: C[C@@H](Nc1ccc(Cl)nc1C(=O)O)c1cc(C(F)F)cc2nc(C#N)c(N3CCOCC3)nc12] | ¹H NMR (400 MHz, CDCl₃) δ = 10.73 (s, 1H), 8.41 (br d, J = 6.4 Hz, 1H), 8.03 (d, J = 1.2 Hz, 1H), 7.80 (d, J = 1.2 Hz, 1H), 7.14 (d, J = 8.8 Hz, 1H), 6.90- 6.54 (m, 2H), 4.04-3.94 (m, 4H), 3.94-3.87 (m, 4H), 1.75 (d, J = 6.8 Hz, 3H). LCMS [M + 23]⁺: 511.1. |
| | (R)-6-chloro-3-((1-(2-cyano-7-(difluoromethyl)-3- | |
| | morpholinoquinoxalin-5-yl)ethyl)amino)picolinic | |
| | acid | |
| 232 | 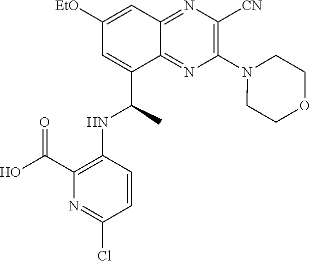 [SMILES: CCOc1cc([C@@H](C)Nc2ccc(Cl)nc2C(=O)O)c2nc(N3CCOCC3)c(C#N)nc2c1] | |
| | (R)-6-chloro-3-((1-(2-cyano-7-ethoxy-3- | |
| | morpholinoquinoxalin-5-yl)ethyl)amino)picolinic | |
| | acid | |
| 233 | 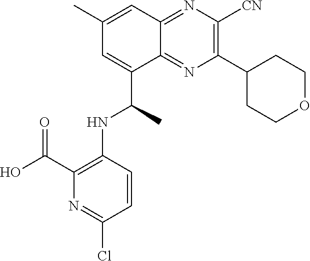 [SMILES: Cc1cc([C@@H](C)Nc2ccc(Cl)nc2C(=O)O)c2nc(C3CCOCC3)c(C#N)nc2c1] | ¹H NMR (400 MHz, CD₃OD) δ = 7.84 (s, 2H), 7.21 (d, J = 9.2 Hz, 1H), 7.06 (d, J = 9.2 Hz, 1H), 5.78 (d, J = 6.8 Hz, 1H), 4.23-4.13 (m, 2H), 3.80-3.64 (m, 3H), 2.57 (s, 3H), 2.25- 2.13 (m, 2H), 2.08-1.98 (m, 2H), 1.79 (d, J = 6.8 Hz, 3H). LCMS [M + 1]+: 452.1. |
| | (R)-6-chloro-3-((1-(2-cyano-7-methyl-3-(tetrahydro- | |
| | 2H-pyran-4-yl)quinoxalin-5-yl)ethyl)amino)picolinic | |
| | acid | |
| 234A and 234B | 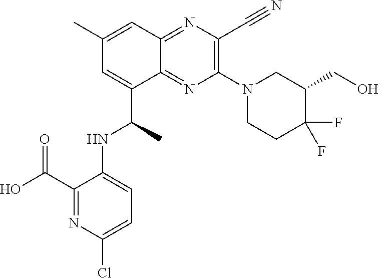 [SMILES: Cc1cc([C@@H](C)Nc2ccc(Cl)nc2C(=O)O)c2nc(N3CCC(F)(F)[C@@H](CO)C3)c(C#N)nc2c1] | 234A, Final product made using the first eluting isomer from SFC before saponification: ¹H NMR (400 MHz, CD₃OD) δ = 7.67 (s, 2H), 7.20 (d, J = 8.8 Hz, 1H), 7.03 (d, J = 9.2 Hz, 1H), 5.61 (q, J = 6.8 Hz, 1H), 4.46 (br d, J = 13.6 Hz, 1H), 4.32-4.20 (m, 1H), 3.99 (dd, J = 4.0, 11.2 Hz, 1H), 3.70-3.56 (m, 2H), 3.48 (dd, J = 10.4, 12.8 Hz, 1H), 2.55- 2.39 (m, 4H), 2.32-2.10 (m, 2H), 1.72 (d, J = 6.4 Hz, 3H). LCMS [M + Na]⁺: 539.1. 234B, Final product made using the second eluting |
| | 6-chloro-3-(((R)-1-(2-cyano-3-((R)-4,4-difluoro-3- | isomer from SFC before |
| | (hydroxymethyl)piperidin-1-yl)-7-methylquinoxalin- | saponification: ¹H NMR (400 |
| | 5-yl)ethyl)amino)picolinic acid | MHz, CD₃OD) = 7.67 (d, J = 2.4 |
| | AND | Hz, 2H), 7.19 (d, J = 9.2 Hz, |
| | 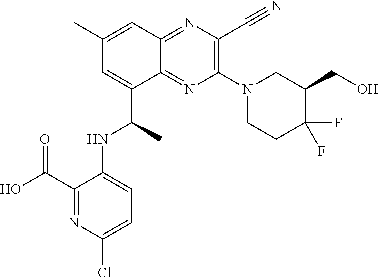 [SMILES: Cc1cc([C@@H](C)Nc2ccc(Cl)nc2C(=O)O)c2nc(N3CCC(F)(F)[C@H](CO)C3)c(C#N)nc2c1] | 1H), 7.01 (d, J = 9.2 Hz, 1H), 5.61 (q, J = 6.8 Hz, 1H), 4.48 (br d, J = 13.2 Hz, 1H), 4.36- 4.19 (m, 1H), 3.99 (dd, J = 4.0, 11.2 Hz, 1H), 3.66-3.54 (m, 2H), 3.52-3.41 (m, 1H), 2.47 (s, 3H) 2.52-2.42 (m, 1H), 2.32- 2.11 (m, 2H), 1.72 (d, J = 6.8 Hz, 3H). LCMS [M + Na]+: 539.2. |
| | 6-chloro-3-(((R)-1-(2-cyano-3-((S)-4,4-difluoro-3- | |
| | (hydroxymethyl)piperidin-1-yl)-7-methylquinoxalin- | |
| | 5-yl)ethyl)amino)picolinic acid | |
| 235A | 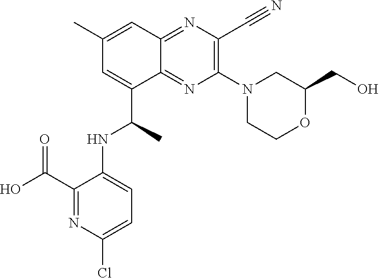 [SMILES: Cc1cc([C@@H](C)Nc2ccc(Cl)nc2C(=O)O)c2nc(N3CCO[C@H](CO)C3)c(C#N)nc2c1] | ¹H NMR (400 MHz, CD₃OD) δ = 7.67 (s, 2H), 7.21 (d, J = 8.8 Hz, 1H), 7.04 (d, J = 9.2 Hz, 1H), 5.55 (q, J = 6.4 Hz, 1H), 4.37- 4.31 (m, 1H), 4.23 (br dd, J = 1.6, 13.2 Hz, 1H), 4.09 (dd, J = 1.6, 11.6 Hz, 1H), 3.91-3.78 (m, 2H), 3.74-3.64 (m, 2H), 3.34-3.33 (m, 1H), 3.08 (dd, J = 10.4, 13.0 Hz, 1H), 2.47 (s, 3H), 1.73 (d, J = 6.4 Hz, 3H). LCMS [M + Na]⁺: 505.2. |
| | 6-chloro-3-(((R)-1-(2-cyano-3-((S)-2- | |
| | (hydroxymethyl)morpholino)-7-methylquinoxalin-5- | |
| | yl)ethyl)amino)picolinic acid | |
| 235B | 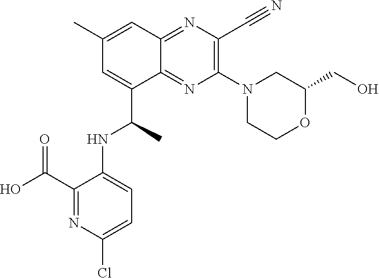 [SMILES: Cc1cc([C@@H](C)Nc2ccc(Cl)nc2C(=O)O)c2nc(N3CCO[C@@H](CO)C3)c(C#N)nc2c1] | ¹H NMR (400 MHz, CD₃OD) δ = 7.66 (s, 2H), 7.20 (d, J = 9.2 Hz, 1H), 7.02 (d, J = 9.2 Hz, 1H), 5.56 (q, J = 6.4 Hz, 1H), 4.32 (br d, J = 12.8 Hz, 1H), 4.25 (br dd, J = 1.6, 13.2 Hz, 1H), 4.10 (dd, J = 1.6, 11.6 Hz, 1H), 3.93- 3.79 (m, 2H), 3.73-3.62 (m, 2H), 3.34-3.33 (m, 1H), 3.16- 3.08 (m, 1H), 2.47 (s, 3H), 1.73 (d, J = 6.8 Hz, 3H). LCMS [M + Na]⁺: 505.2. |
| | 6-chloro-3-(((R)-1-(2-cyano-3-((R)-2- | |
| | (hydroxymethyl)morpholino)-7-methylquinoxalin-5- | |
| | yl)ethyl)amino)picolinic acid | |
| 236A | 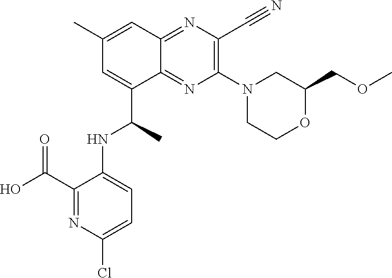 [SMILES: COC[C@@H]1CN(c2nc3c([C@@H](C)Nc4ccc(Cl)nc4C(=O)O)cc(C)cc3nc2C#N)CCO1] | ¹H NMR (400 MHz, CD₃OD) δ = 7.66 (s, 1H), 7.65 (s, 1H), 7.20 (d, J = 9.2 Hz, 1H), 7.05 (d, J = 9.2 Hz, 1H), 5.53 (q, J = 6.8 Hz, 1H), 4.31 (td, J = 2.0, 13.2 Hz, 1H), 4.22 (dd, J = 2.0, 13.2 Hz, 1H), 4.10-4.04 (m, 1H), 3.93 (dtd, J = 2.4, 5.2, 10.4 Hz, 1H), 3.86 (dt, J = 2.4, 11.6 Hz, 1H), 3.61-3.52 (m, 2H), 3.41 (s, 3H), 3.38-3.32 (m, 1H), 3.08 (dd, J = 10.4, 13.2 Hz, 1H), 2.46 (s, 3H), 1.73 (d, J = 6.8 Hz, 3H). LCMS [M + H]⁺: 497.2. |
| | 6-chloro-3-(((R)-1-(2-cyano-3-((S)-2- | |
| | (methoxymethyl)morpholino)-7-methylquinoxalin-5- | |
| | yl)ethyl)amino)picolinic acid | |
| 236B | 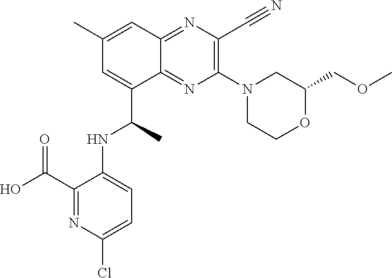 [SMILES: COC[C@H]1CN(c2nc3c([C@@H](C)Nc4ccc(Cl)nc4C(=O)O)cc(C)cc3nc2C#N)CCO1] | ¹H NMR (400 MHz, CD₃OD) δ = 7.69 (s, 1H), 7.68(s, 1H), 7.19 (d, J = 9.2 Hz, 1H), 7.02 (d, J = 9.2 Hz, 1H), 5.57 (q, J = 6.4 Hz, 1H), 4.39-4.22 (m, 2H), 4.15- 4.05 (m, 1H), 4.01-3.80 (m, 2H), 3.57 (dq, J = 5.2, 10.4 Hz, 2H), 3.42 (s, 3H), 3.34-3.27 (m, 1H), 3.15 (dd, J = 10.4, 13.2 Hz, 1H), 2.49 (s, 3H), 1.74 (d, J = 6.8 Hz, 3H). LCMS [M + H]⁺: 497.1. |
| | 6-chloro-3-(((R)-1-(2-cyano-3-((R)-2- | |
| | (methoxymethyl)morpholino)-7-methylquinoxalin-5- | |
| | yl)ethyl)amino)picolinic acid | |
| 237A | 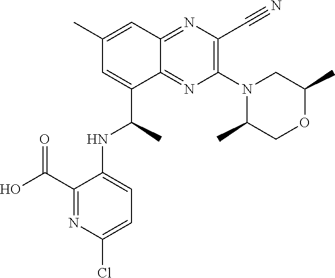 [SMILES: Cc1cc([C@@H](C)Nc2ccc(Cl)nc2C(=O)O)c2nc(N3C[C@@H](C)OC[C@H]3C)c(C#N)nc2c1] | ¹H NMR (400 MHz, CD₃OD) δ = 7.64 (d, J = 1.6 Hz, 1H), 7.62 (s, 1H), 7.13 (d, J = 8.8 Hz, 1H), 6.90 (d, J = 8.8 Hz, 1H), 5.55- 5.48 (m, 1H), 4.62-4.58 (m, 1H), 4.14 (dd, J = 2.0, 13.6 Hz, 1H), 3.94-3.90 (m, 1H), 3.85- 3.79 (m, 2H), 3.21 (dd, J = 11.2, 13.6 Hz, 1H), 2.44 (s, 3H), 1.70 (d, J = 6.8 Hz, 3H), 1.44 (d, J = 6.8 Hz, 3H), 1.29 (d, J = 6.0 Hz, 3H). LCMS [M + Na]⁺ = 503.2. |
| | 6-chloro-3-(((R)-1-(2-cyano-3-((2R,5R)-2,5- | |
| | dimethylmorpholino)-7-methylquinoxalin-5- | |
| | yl)ethyl)amino)picolinic acid | |
| 237B | 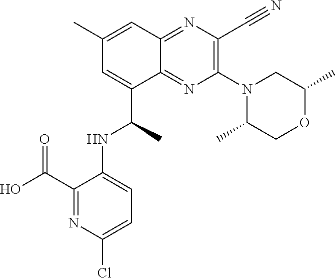 [SMILES: Cc1cc([C@@H](C)Nc2ccc(Cl)nc2C(=O)O)c2nc(N3C[C@H](C)OC[C@@H]3C)c(C#N)nc2c1] | ¹H NMR (400 MHz, CD₃OD) δ = 7.67 (s, 1H), 7.61 (s, 1H), 7.10 (d, J = 8.8 Hz, 1H), 6.90 (d, J = 8.8 Hz, 1H), 5.49 (q, J = 6.8 Hz, 1H), 4.61-4.58 (m, 1H), 4.14 (dd, J = 1.6, 13.6 Hz, 1H), 3.95-3.91 (m, 1H), 3.84- 3.79 (m, 2H), 3.27-3.24 (m, 1H), 2.45 (s, 3H), 1.70 (d, J = 6.8 Hz, 3H), 1.45 (d, J = 6.8 Hz, 3H), 1.28 (d, J = 6.0 Hz, 3H). LCMS [M + Na]⁺ = 503.2. |
| | 6-chloro-3-(((R)-1-(2-cyano-3-((2S,5S)-2,5- | |
| | dimethylmorpholino)-7-methylquinoxalin-5- | |
| | yl)ethyl)amino)picolinic acid | |
| 238A | 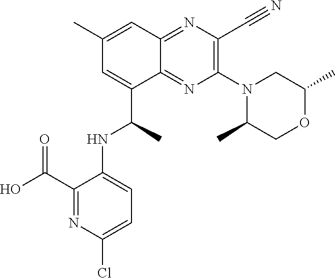 [SMILES: Cc1cc([C@@H](C)Nc2ccc(Cl)nc2C(=O)O)c2nc(N3C[C@H](C)OC[C@H]3C)c(C#N)nc2c1] | ¹H NMR (400 MHz, CD₃OD) δ = 7.74 (br t, J = 2.0 Hz, 2H), 7.21-7.14 (m, 1H), 6.93 (d, J = 9.2 Hz, 1H), 5.70 (q, J = 6.4 Hz, 1H), 4.07-3.98 (m, 2H), 3.87- 3.83 (m, 1H), 3.75 (dd, J = 2.4, 12.4 Hz, 1H), 3.58-3.53 (m, 1H), 2.93 (dd, J = 8.4, 12.4 Hz, 1H), 2.53-2.46 (m, 3H), 1.69 (d, J = 6.4 Hz, 3H), 1.26 (d, J = 6.4 Hz, 3H), 1.15 (d, J = 6.0 Hz, 3H). LCMS [M + Na]⁺ = 503.1. |
| | 6-chloro-3-(((R)-1-(2-cyano-3-((2S,5R)-2,5- | |
| | dimethylmorpholino)-7-methylquinoxalin-5- | |
| | yl)ethyl)amino)picolinic acid | |
| 238B | 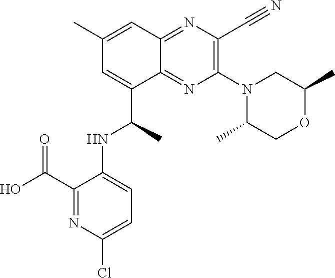 [SMILES: Cc1cc([C@@H](C)Nc2ccc(Cl)nc2C(=O)O)c2nc(N3C[C@@H](C)OC[C@@H]3C)c(C#N)nc2c1] | ¹H NMR (400 MHz, CD₃OD) δ = 7.78-7.72 (m, 2H), 7.20 (d, J = 9.2 Hz, 1H), 7.03 (d, J = 9.2 Hz, 1H), 5.57 (q, J = 6.4 Hz, 1H), 4.07-3.98 (m, 2H), 3.95- 3.86 (m, 1H), 3.72 (dd, J = 2.4, 12.4 Hz, 1H), 3.54 (dd, J = 9.2, 11.6 Hz, 1H), 2.94 (dd, J = 8.8, 12.4 Hz, 1H), 2.51 (s, 3H), 1.76 (d, J = 6.4 Hz, 3H), 1.26 (d, J = 6.4 Hz, 3H), 1.14 (d, J = 6.4 Hz, 3H). LCMS [M + Na]⁺ = 503.1. |
| | 6-chloro-3-(((R)-1-(2-cyano-3-((2R,5S)-2,5- | |
| | dimethylmorpholino)-7-methylquinoxalin-5- | |
| | yl)ethyl)amino)picolinic acid | |
| 239A and 239B | 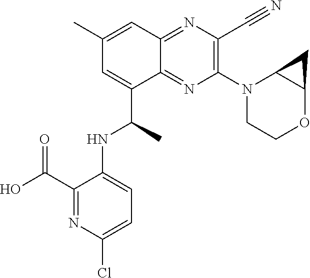 [SMILES: Cc1cc([C@@H](C)Nc2ccc(Cl)nc2C(=O)O)c2nc(N3CCO[C@@H]4C[C@@H]43)c(C#N)nc2c1] | Purified by SFC prior to saponification: First eluting isomer leading to final compound 239A: ¹H NMR (400 MHz, CD₃OD) δ = 7.61 (br d, J = 5.6 Hz, 2H), 7.20 (d, J = 8.8 Hz, 1H), 7.02 (d, J = 9.2 Hz, 1H), 5.54 (q, J = 6.4 Hz, 1H), 4.03-3.95 (m, 4H), 3.54 (td, J = 3.2, 12.4 Hz, 1H), 3.37-3.34 (m, 1H), 2.45 (s, 3H), 1.72 (d, J = 6.8 Hz, 3H), 1.17 (q, J = 6.8 Hz, 1H), 1.04-1.00 (m, 1H). LCMS [M + Na]⁺ = 487.2. Second eluting isomer leading to final compound 239B: ¹H NMR (400 MHz, CD₃OD) δ = |
| | 3-(((R)-1-(3-((1R,6S)-2-oxa-5- | 7.61 (s, 2H), 7.20 (d, J = 8.8 Hz, |
| | azabicyclo[4.1.0]heptan-5-yl)-2-cyano-7- | 1H), 7.01 (d, J = 8.8 Hz, 1H), |
| | methylquinoxalin-5-yl)ethyl)amino)-6-chloropicolinic | 5.54 (q, J = 6.4 Hz, 1H), 4.04 |
| | acid | (dt, J = 3.6, 6.4 Hz, 1H), 3.98- |
| | AND | 3.92 (m, 3H), 3.63-3.57 (m, |
| | 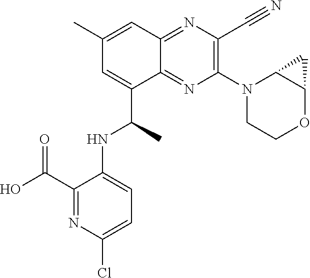 [SMILES: Cc1cc([C@@H](C)Nc2ccc(Cl)nc2C(=O)O)c2nc(N3CCO[C@H]4C[C@H]43)c(C#N)nc2c1] | 1H), 3.38-3.35 (m, 1H), 2.45 (s, 3H), 1.73 (d, J = 6.8 Hz, 3H), 1.18 (q, J = 6.8 Hz, 1H), 1.02- 0.98 (m, 1H). LCMS [M + Na]⁺ = 487.2. |
| | 3-(((R)-1-(3-((1S,6R)-2-oxa-5- | |
| | azabicyclo[4.1.0]heptan-5-yl)-2-cyano-7- | |
| | methylquinoxalin-5-yl)ethyl)amino)-6-chloropicolinic | |
| | acid | |
| 240 | 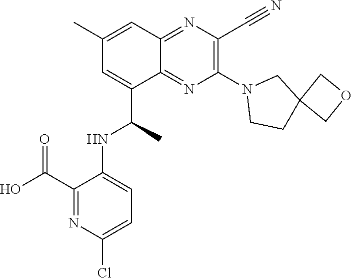 [SMILES: Cc1cc([C@@H](C)Nc2ccc(Cl)nc2C(=O)O)c2nc(N3CCC4(COC4)C3)c(C#N)nc2c1] | ¹H NMR (400 MHz, CDCl₃) δ = 8.37 (br d, J = 6.8 Hz, 1H), 7.59 (s, 1H), 7.47 (d, J J = 1.6 Hz, 1H), 7.11 (d, J = 8.8 Hz, 1H), 6.79 (d, J = 9.2 Hz, 1H), 5.48- 5.39 (m, 1H), 4.84-4.76 (m, 2H), 4.71 (dd, J = 1.2, 6.4 Hz, 2H), 4.20-4.09 (m, 2H), 4.05- 3.93 (m, 2H), 2.48-2.38 (m, 5H), 1.72 (d, J = 6.8 Hz, 3H). LCMS [M + Na]⁺ = 501.2. |
| | (R)-6-chloro-3-((1-(2-cyano-7-methyl-3-(2-oxa-6- | |
| | azaspiro[3.4]octan-6-yl)quinoxalin-5- | |
| | yl)ethyl)amino)picolinic acid | |
| 241A and 241B | 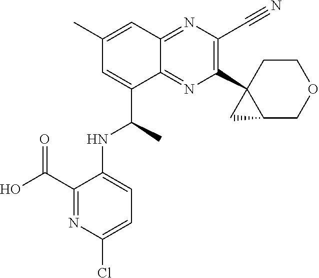 [SMILES: Cc1cc([C@@H](C)Nc2ccc(Cl)nc2C(=O)O)c2nc([C@@]34CCOC[C@@H]3C4)c(C#N)nc2c1] | |
| | 3-(((R)-1-(3-((1R,6S)-3-oxabicyclo[4.1.0]heptan-6-yl)- | |
| | 2-cyano-7-methylquinoxalin-5-yl)ethyl)amino)-6- | |
| | chloropicolinic acid | |
| | AND | |
| | 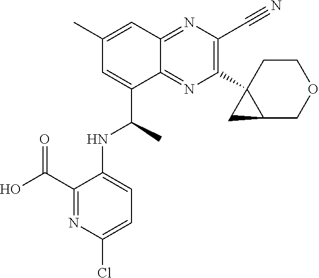 [SMILES: Cc1cc([C@@H](C)Nc2ccc(Cl)nc2C(=O)O)c2nc([C@]34CCOC[C@H]3C4)c(C#N)nc2c1] | |
| | 3-(((R)-1-(3-((1S,6R)-3-oxabicyclo[4.1.0]heptan-6-yl)- | |
| | 2-cyano-7-methylquinoxalin-5-yl)ethyl)amino)-6- | |
| | chloropicolinic acid | |
| 242 | 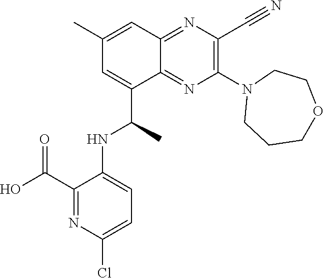 [SMILES: Cc1cc([C@@H](C)Nc2ccc(Cl)nc2C(=O)O)c2nc(N3CCCOCC3)c(C#N)nc2c1] | ¹H NMR (400 MHz, CD₃OD) δ = 7.59 (s, 2H), 7.19 (d, J = 9.2 Hz, 1H), 6.95 (d, J = 9.2 Hz, 1H), 5.52 (q, J = 6.8 Hz, 1H), 4.18-4.09 (m, 4H), 4.03-3.96 (m, 2H), 3.84 (t, J = 5.6 Hz, 2H), 2.43 (s, 3H), 2.21-2.11 (m, 2H), 1.69 (d, J = 6.8 Hz, 3H). LCMS [M + 1]⁺: 467.2. |
| | (R)-6-chloro-3-((1-(2-cyano-7-methyl-3-(1,4- | |
| | oxazepan-4-yl)quinoxalin-5-yl)ethyl)amino)picolinic | |
| | acid | |
| 243A and 243B | 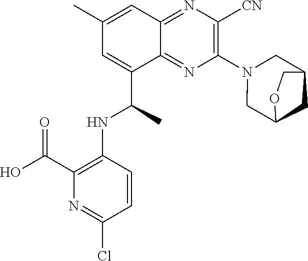 | 243A, Final product made using the first eluting isomer from SFC before saponification: ¹H NMR (400 MHz, CDCl₃) δ = 8.26-8.20 (m, 1H), 7.53 (s, 1H), 7.40 (br s, 1H), 7.15-7.02 (m, 1H), 6.69- 6.56 (m, 1H), 5.44 (br d, J = 4.2 Hz, 1H), 4.71 (br d, J = 11.6 Hz, 1H), 4.45 (br s, 1H), 4.19 (br d, J = 11.2 Hz, 1H), 4.06 (br d, J = 5.2 Hz, 1H), 3.89 (br s, 1H), 3.39-3.34 (m, 2H), 2.68 (br s, 1H), 2.36 (br s, 3H), 2.04 (br s, 1H), 1.91 (br d, J = 11.2 Hz, 1H), 1.62 (br s, 3H). LCMS |
| | 3-(((R)-1-(3-((1R,5S)-6-oxa-3-azabicyclo[3.2.1]octan- | [M + Na]⁺ = 501.1. |
| | 3-yl)-2-cyano-7-methylquinoxalin-5-yl)ethyl)amino)- | 243B, Final product made |
| | 6-chloropicolinic acid | using the second eluting |
| | AND | isomer from SFC before |
| | 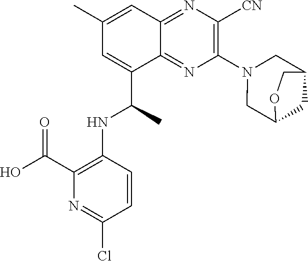 [SMILES: Cc1cc([C@@H](C)Nc2ccc(Cl)nc2C(=O)O)c2nc(N3C[C@H]4CO[C@H](C4)C3)c(C#N)nc2c1] | saponification: ¹H NMR (400 MHz, CDCl₃) δ = 8.28 (br d, J = 1.6 Hz, 1H), 7.53 (s, 1H), 7.42 (s, 1H), 7.04 (br d, J = 7.6 Hz, 1H), 6.71 (br d, J = 8.8 Hz, 1H), 5.39 (br t, J = 6.0 Hz, 1H), 4.66 (br d, J = 12.0 Hz, 1H), 4.47- 4.45 (m, 1H), 4.27-4.24 (m, 1H), 4.03 (d, J = 8.0 Hz, 1H), 3.86-3.83 (m, 1H), 3.42 (br d, J = 12.8 Hz, 1H), 3.35 (br d, J = 12.8 Hz, 1H), 2.68 (br s, 1H), 2.36 (s, 3H), 2.07-2.04 (m, 1H), 1.94 (br d, J = 11.2 Hz, 1H), 1.63 (br d, J = 6.4 Hz, 3H). LCMS [M + Na]⁺ = 501.1. |
| | 3-(((R)-1-(3-((1S,5R)-6-oxa-3-azabicyclo[3.2.1]octan- | |
| | 3-yl)-2-cyano-7-methylquinoxalin-5-yl)ethyl)amino)- | |
| | 6-chloropicolinic acid | |
| 244A and 244B | 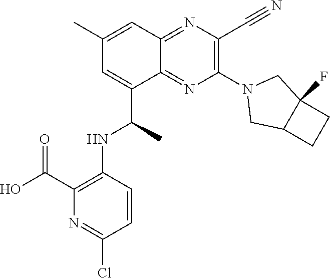 [SMILES: Cc1cc([C@@H](C)Nc2ccc(Cl)nc2C(=O)O)c2nc(N3CC4CC[C@@]4(F)C3)c(C#N)nc2c1] | 244A, Final product made using the first eluting isomer from SFC before saponification: ¹H NMR (400 MHz, CD₃OD) δ = 7.58 (br d, J = 2.8 Hz, 2H), 7.20 (d, J = 9.2 Hz, 1H), 7.02 (d, J = 9.2 Hz, 1H), 5.53 (q, J = 6.4 Hz, 1H), 4.45 (dd, J = 13.2, 16.4 Hz, 1H), 4.27-4.13 (m, 2H), 4.12-4.03 (m, 1H), 3.29-3.19 (m, 1H), 2.57-2.35 (m, 5H), 2.30-2.14 (m, 1H), 1.72 (d, J = 6.4 Hz, 3H), 1.53-1.36 (m, 1H). LCMS [M + Na]⁺ = 503.1. 244B, Final product made using the second eluting |
| | 6-chloro-3-(((1R)-1-(2-cyano-3-((1S)-1-fluoro-3- | isomer from SFC before |
| | azabicyclo[3.2.0]heptan-3-yl)-7-methylquinoxalin-5- | saponification: ¹H NMR (400 |
| | yl)ethyl)amino)picolinic acid | MHz, CD₃OD) δ = 7.67-7.52 |
| | AND | (m, 2H), 7.19 (d, J = 9.2 Hz, |
| | 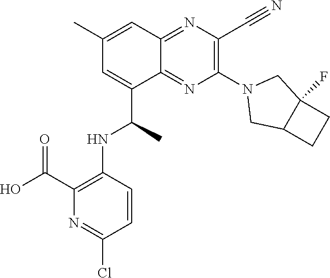 [SMILES: Cc1cc([C@@H](C)Nc2ccc(Cl)nc2C(=O)O)c2nc(N3CC4CC[C@]4(F)C3)c(C#N)nc2c1] | 1H), 7.04 (d, J = 9.2 Hz, 1H), 5.48 (q, J = 6.8 Hz, 1H), 4.53 (dd, J = 13.2, 16.8 Hz, 1H), 4.25-4.06 (m, 3H), 3.30-3.19 (m, 1H), 2.57-2.39 (m, 5H), 2.28- 2.14 (m, 1H), 1.74 (d, J = 6.8 Hz, 3H), 1.51-1.37 (m, 1H). LCMS [M + Na]⁺ = 503.1. |
| | 6-chloro-3-(((1R)-1-(2-cyano-3-((1S)-1-fluoro-3- | |
| | azabicyclo[3.2.0]heptan-3-yl)-7-methylquinoxalin-5- | |
| | yl)ethyl)amino)picolinic acid | |
| 245 | 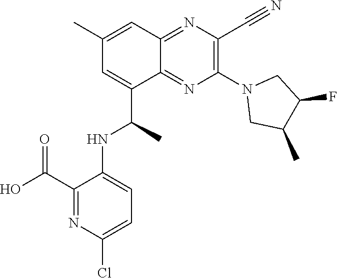 [SMILES: Cc1cc([C@@H](C)Nc2ccc(Cl)nc2C(=O)O)c2nc(N3C[C@@H](F)[C@@H](C)C3)c(C#N)nc2c1] | ¹H NMR (400 MHz, CDCl₃)δ = 8.35 (br dd, J = 5.6, 13.6 Hz, 1H), 7.59 (s, 1H), 7.47 (d, J = 2.0 Hz, 1H), 7.11 (dd, J = 4.8, 9.2 Hz, 1H), 6.79 (dd, J = 2.8, 8.8 Hz, 1H), 5.58-5.34 (m, 1H), 5.28-5.06 (m, 1H), 4.33- 4.05 (m, 3H), 3.69 (td, J = 11.2, 18.8 Hz, 1H), 2.60-2.45 (m, 1H), 2.43 (s, 3H), 1.70 (dd, J = 6.8, 10.4 Hz, 4H), 1.32 (d, J = 6.8 Hz, 3H). LCMS [M + Na]+: 491.1. |
| | 6-chloro-3-(((R)-1-(2-cyano-3-((3S,4S)-3-fluoro-4- | |
| | methylpyrrolidin-1-yl)-7-methylquinoxalin-5- | |
| | yl)ethyl)amino)picolinic acid | |
| 246A | 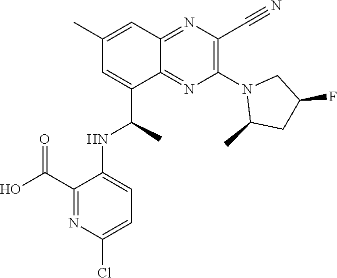 [SMILES: Cc1cc([C@@H](C)Nc2ccc(Cl)nc2C(=O)O)c2nc(N3C[C@@H](F)C[C@H]3C)c(C#N)nc2c1] | ¹H NMR (400 MHz, CD₃OD) δ = 7.56 (s, 2H), 7.17 (d, J = 8.8 Hz, 1H), 6.91 (d, J = 9.2 Hz, 1H), 5.64-5.37 (m, 2H), 4.96- 4.92 (m, 1H), 4.52-4.36 (m, 1H), 4.31-4.16 (m, 1H), 2.52- 2.34 (m, 4H), 2.20 (br dd, J = 14.8, 20.0 Hz, 1H), 1.68 (d, J = 6.8 Hz, 3H), 1.51 (d, J = 6.4 Hz, 3H). LCMS [M + Na]+: 491.1. |
| | 6-chloro-3-(((R)-1-(2-cyano-3-((2R,4S)-4-fluoro-2- | |
| | methylpyrrolidin-1-yl)-7-methylquinoxalin-5- | |
| | yl)ethyl)amino)picolinic acid | |
| 246B | 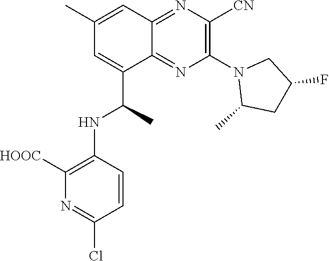 [SMILES: Cc1cc([C@@H](C)Nc2ccc(Cl)nc2C(=O)O)c2nc(N3C[C@H](F)C[C@@H]3C)c(C#N)nc2c1] | ¹H NMR (400 MHz, CD₃OD) δ = 7.58 (s, 1H), 7.56 (s, 1H), 7.19 (d, J = 9.2 Hz, 1H), 6.99 (d, J = 9.2 Hz, 1H), 5.57-5.37 (m, 2H), 5.07-5.00 (m, 1H), 4.53-4.36 (m, 1H), 4.28-4.11 (m, 1H), 2.55-2.33 (m, 4H), 2.28-2.12 (m, 1H), 1.73 (d, J = 6.8 Hz, 3H), 1.51 (d, J = 6.4 Hz, 3H). LCMS [M + Na]⁺: 491.2. |
| | 6-chloro-3-(((R)-1-(2-cyano-3-((2S,4R)-4-fluoro-2- | |
| | methylpyrrolidin-1-yl)-7-methylquinoxalin-5- | |
| | yl)ethyl)amino)picolinic acid | |
| 246C | 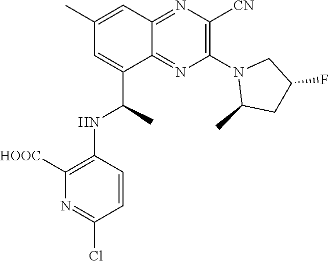 [SMILES: Cc1cc([C@@H](C)Nc2ccc(Cl)nc2C(=O)O)c2nc(N3C[C@H](F)C[C@H]3C)c(C#N)nc2c1] | ¹H NMR (400 MHz, CD₃OD) δ = 7.56 (s, 2H), 7.17 (d, J = 9.2 Hz, 1H), 6.91 (d, J = 9.2 Hz, 1H), 5.61-5.37 (m, 2H), 4.98- 4.92 (m, 1H), 4.50-4.36 (m, 1H), 4.31-4.13 (m, 1H), 2.53- 2.33 (m, 4H), 2.16-2.25 (m, 1H), 1.68 (d, J = 6.8 Hz, 3H), 1.51 (d, J = 6.4 Hz, 3H). LCMS [M + Na]⁺: 491.1. |
| | 6-chloro-3-(((R)-1-(2-cyano-3-((2R,4R)-4-fluoro-2- | |
| | methylpyrrolidin-1-yl)-7-methylquinoxalin-5- | |
| | yl)ethyl)amino)picolinic acid | |
| 246D | 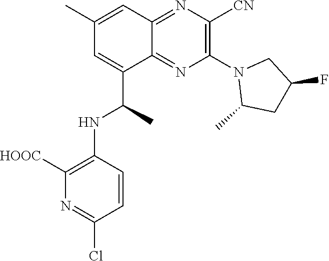 [SMILES: Cc1cc([C@@H](C)Nc2ccc(Cl)nc2C(=O)O)c2nc(N3C[C@@H](F)C[C@@H]3C)c(C#N)nc2c1] | ¹H NMR (400 MHz, CD₃OD) δ = 7.61 (s, 2H), 7.19 (d, J = 9.2 Hz, 1H), 6.94 (d, J = 9.2 Hz, 1H), 5.56 (q, J = 6.8 Hz, 1H), 5.47-5.28 (m, 1H), 4.82-4.74 (m, 1H), 4.38-4.21 (m, 1H), 4.19-4.06 (m, 1H), 2.67-2.54 (m, 1H), 2.44 (s, 3H), 2.12- 1.92 (m, 1H), 1.73 (d, J = 6.4 Hz, 3H), 1.46 (d, J = 6.0 Hz, 3H). LCMS [M + Na]⁺: 491.1. |
| | 6-chloro-3-(((R)-1-(2-cyano-3-((2S,4S)-4-fluoro-2- | |
| | methylpyrrolidin-1-yl)-7-methylquinoxalin-5- | |
| | yl)ethyl)amino)picolinic acid | |
| 247A and 247B | 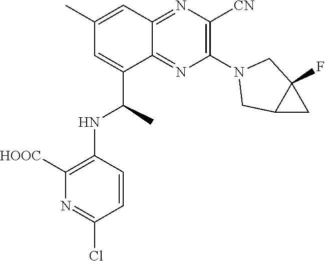 [SMILES: Cc1cc([C@@H](C)Nc2ccc(Cl)nc2C(=O)O)c2nc(N3CC4C[C@@]4(F)C3)c(C#N)nc2c1] | |
| | 6-chloro-3-(((1R)-1-(2-cyano-3-((1S)-1-fluoro-3- | |
| | azabicyclo[3.1.0]hexan-3-yl)-7-methylquinoxalin-5- | |
| | yl)ethyl)amino)picolinic acid | |
| | AND | |
| | 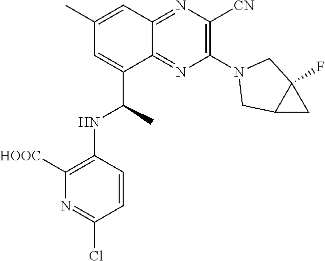 [SMILES: Cc1cc([C@@H](C)Nc2ccc(Cl)nc2C(=O)O)c2nc(N3CC4C[C@]4(F)C3)c(C#N)nc2c1] | |
| | 6-chloro-3-(((1R)-1-(2-cyano-3-((1R)-1-fluoro-3- | |
| | azabicyclo[3.1.0]hexan-3-yl)-7-methylquinoxalin-5- | |
| | yl)ethyl)amino)picolinic acid | |
| 248A and 248B | 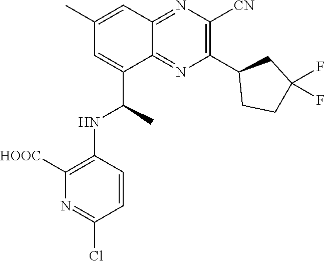 [SMILES: Cc1cc([C@@H](C)Nc2ccc(Cl)nc2C(=O)O)c2nc([C@@H]3CCC(F)(F)C3)c(C#N)nc2c1] | |
| | 6-chloro-3-(((R)-1-(2-cyano-3-((R)-3,3- | |
| | difluorocyclopentyl)-7-methylquinoxalin-5- | |
| | yl)ethyl)amino)picolinic acid | |
| | AND | |
| | 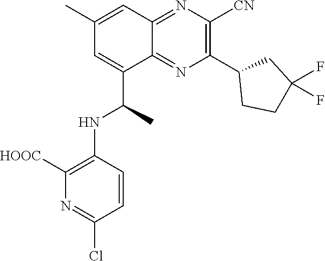 [SMILES: Cc1cc([C@@H](C)Nc2ccc(Cl)nc2C(=O)O)c2nc([C@H]3CCC(F)(F)C3)c(C#N)nc2c1] | |
| | 6-chloro-3-(((R)-1-(2-cyano-3-((S)-3,3- | |
| | difluorocyclopentyl)-7-methylquinoxalin-5- | |
| | yl)ethyl)amino)picolinic acid | |
| 249 | 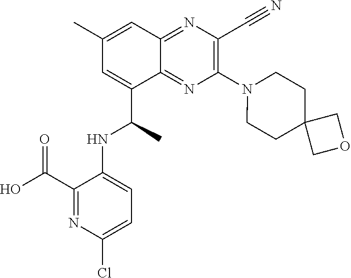 [SMILES: Cc1cc([C@@H](C)Nc2ccc(Cl)nc2C(=O)O)c2nc(N3CCC4(CC3)COC4)c(C#N)nc2c1] | ¹H NMR (400 MHz, CD₃OD) δ = 7.71-7.57 (m, 2H), 7.20 (d, J = 9.2 Hz, 1H), 7.03 (d, J = 9.2 Hz, 1H), 5.52 (q, J = 6.8 Hz, 1H), 4.55 (s, 4H), 3.79-3.59 (m, 4H), 2.45 (s, 3H), 2.12 (t, J = 5.6 Hz, 4H), 1.72 (d, J = 6.8 Hz, 3H). LCMS [M + 1]⁺ = 493.2. |
| | (R)-6-chloro-3-((1-(2-cyano-7-methyl-3-(2-oxa-7- | |
| | azaspiro[3.5]nonan-7-yl)quinoxalin-5- | |
| | yl)ethyl)amino)picolinic acid | |
| 250 | 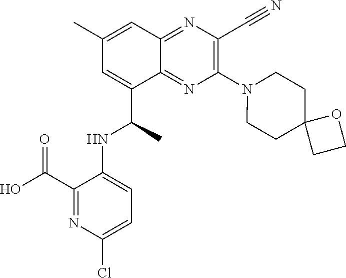 [SMILES: Cc1cc([C@@H](C)Nc2ccc(Cl)nc2C(=O)O)c2nc(N3CCC4(CCO4)CC3)c(C#N)nc2c1] | ¹H NMR (400 MHz, CD₃OD) δ = 7.64 (s, 2H), 7.19 (d, J = 8.8 Hz, 1H), 7.00 (d, J = 8.8 Hz, 1H), 5.55 (q, J = 6.4 Hz, 1H), 4.62 (t, J = 8.0 Hz, 2H), 3.87- 3.70 (m, 4H), 2.55 (t, J = 8.0 Hz, 2H), 2.45 (s, 3H), 2.20-2.06 (m, 4H), 1.71 (d, J = 6.4 Hz, 3H). LCMS [M + 1]⁺ = 493.3. |
| | (R)-6-chloro-3-((1-(2-cyano-7-methyl-3-(1-oxa-7- | |
| | azaspiro[3.5]nonan-7-yl)quinoxalin-5- | |
| | yl)ethyl)amino)picolinic acid | |
| 251 | 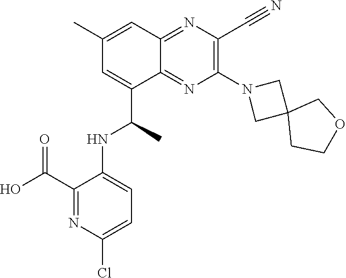 [SMILES: Cc1cc([C@@H](C)Nc2ccc(Cl)nc2C(=O)O)c2nc(N3CC4(CCOC4)C3)c(C#N)nc2c1] | ¹H NMR (400 MHz, CDCl₃) δ = 10.95-10.41 (m, 1H), 8.37 (br d, J = 6.4 Hz, 1H), 7.59 (s, 1H), 7.48 (d, J = 1.6 Hz, 1H), 7.12 (d, J = 9.2 Hz, 1H), 6.80 (d, J = 9.2 Hz, 1H), 5.43 (quin, J = 6.8 Hz, 1H), 4.55-4.41 (m, 4H), 4.04-3.98 (m, 2H), 3.94 (t, J = 6.8 Hz, 2H), 2.44 (s, 3H), 2.36- 2.26 (m, 2H), 1.70 (d, J = 6.8 Hz, 3H). LCMS [M + Na]⁺: 501.1. |
| | (R)-6-chloro-3-((1-(2-cyano-7-methyl-3-(6-oxa-2- | |
| | azaspiro[3.4]octan-2-yl)quinoxalin-5- | |
| | yl)ethyl)amino)picolinic acid | |
| 252 | 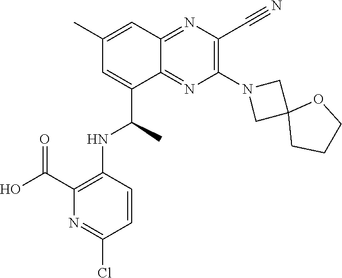 [SMILES: Cc1cc([C@@H](C)Nc2ccc(Cl)nc2C(=O)O)c2nc(N3CC4(CCCO4)C3)c(C#N)nc2c1] | ¹H NMR (400 MHz, CD₃OD) δ = 7.62-7.52 (m, 2H), 7.21 (d, J = 9.2 Hz, 1H), 7.04 (d, J = 9.2 Hz, 1H), 5.49 (q, J = 6.8 Hz, 1H), 4.57-4.36 (m, 4H), 3.93 (t, J = 6.8 Hz, 2H), 2.43 (s, 3H), 2.29-2.20 (m, 2H), 2.06-1.99 (m, 2H), 1.71 (d, J = 6.8 Hz, 3H).LCMS [M + Na]⁺: 501.2. |
| | (R)-6-chloro-3-((1-(2-cyano-7-methyl-3-(5-oxa-2- | |
| | azaspiro[3.4]octan-2-yl)quinoxalin-5- | |
| | yl)ethyl)amino)picolinic acidS | |
| 253A and 253B | 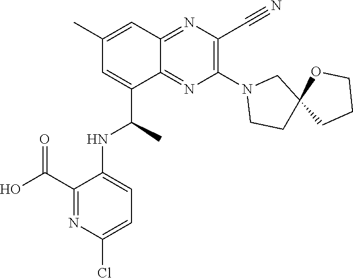 [SMILES: Cc1cc([C@@H](C)Nc2ccc(Cl)nc2C(=O)O)c2nc(N3CC[C@@]4(CCCO4)C3)c(C#N)nc2c1] | Purified by SFC prior to saponification: First eluting isomer leading to final compound 253A: ¹H NMR (400 MHz, CD₃OD) δ = 7.55 (dd, J = 1.6, 10.4 Hz, 2H), 7.19 (d, J = 9.2 Hz, 1H), 7.02 (d, J = 9.2 Hz, 1H), 5.50 (q, J = 6.8 Hz, 1H), 4.14-4.01 (m, 2H), 3.99- 3.83 (m, 4H), 2.41 (s, 3H), 2.26- 2.02 (m, 6H), 1.71 (d, J = 6.8 Hz, 3H). LCMS [M + 1]⁺ = 493.2. Second eluting isomer leading to final compound 253B: ¹H NMR (400 MHz, CD₃OD) δ = 7.61-7.48 (m, 2H), 7.19 (d, J = 9.2 Hz, 1H), 7.04 (d, J = 9.2 Hz, |
| | 6-chloro-3-(((R)-1-(2-cyano-7-methyl-3-((S)-1-oxa-7- | 1H), 5.50 (q, J = 6.8 Hz, 1H), |
| | azaspiro[4.4]nonan-7-yl)quinoxalin-5- | 4.13-3.82 (m, 6H), 2.41 (s, |
| | yl)ethyl)amino)picolinic acid | 3H), 2.24-2.01 (m, 6H), 1.71 |
| | AND | (d, J = 6.8 Hz, 3H). LCMS |
| | 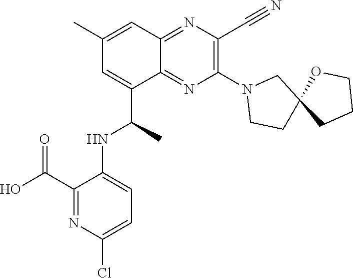 [SMILES: Cc1cc([C@@H](C)Nc2ccc(Cl)nc2C(=O)O)c2nc(N3CC[C@]4(CCCO4)C3)c(C#N)nc2c1] | [M + 1]⁺ = 493.2. |
| | 6-chloro-3-(((R)-1-(2-cyano-7-methyl-3-((R)-1-oxa-7- | |
| | azaspiro[4.4]nonan-7-yl)quinoxalin-5- | |
| | yl)ethyl)amino)picolinic acid | |
| 254A and 254B | 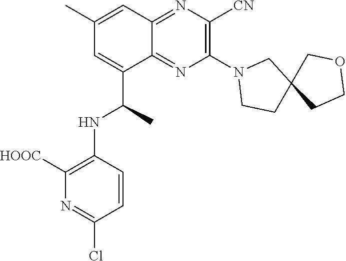 [SMILES: Cc1cc([C@@H](C)Nc2ccc(Cl)nc2C(=O)O)c2nc(N3CC[C@]4(CCOC4)C3)c(C#N)nc2c1] | Purified by SFC prior to saponification: First eluting isomer leading to final compound 254A: ¹H NMR (400 MHz, CD₃OD) δ = 7.57 (d, J = 1.8 Hz, 1H), 7.55 (s, 1H), 7.21 (d, J = 9.2 Hz, 1H), 7.05 (d, J = 9.2 Hz, 1H), 5.51-5.46 (m, 1H), 4.10 (td, J = 6.8, 10.8 Hz, 1H), 4.03-3.93 (m, 4H), 3.87-3.78 (m, 2H), 3.76-3.70 (m, 1H), 2.42 (s, 3H), 2.20- 2.02 (m, 4H), 1.73 (d, J = 6.8 Hz, 3H). LCMS [M + H]⁺: 493.1. Second eluting isomer leading to final compound 254B: ¹H |
| | 6-chloro-3-(((R)-1-(2-cyano-7-methyl-3-((S)-2-oxa-7- | NMR (400 MHz, CD₃OD) δ = |
| | azaspiro[4.4]nonan-7-yl)quinoxalin-5- | 7.57 (d, J = 1.6 Hz, 1H), 7.54 (s, |
| | yl)ethyl)amino)picolinic acid | 1H), 7.18 (d, J = 9.2 Hz, 1H), |
| | AND | 7.03 (d, J = 9.2 Hz, 1H), 5.48 |
| | 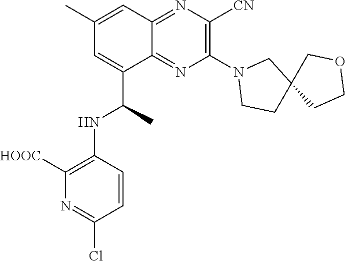 [SMILES: Cc1cc([C@@H](C)Nc2ccc(Cl)nc2C(=O)O)c2nc(N3CC[C@@]4(CCOC4)C3)c(C#N)nc2c1] | (q, J = 6.8 Hz, 1H), 4.04 (t, J = 6.8 Hz, 2H), 3.97 (t, J = 7.2 Hz, 2H), 3.94-3.84 (m, 2H), 3.80- 3.70 (m, 2H), 2.42 (s, 3H), 2.17- 1.99 (m, 4H), 1.72 (d, J = 6.8 Hz, 3H). LCMS [M + H]⁺: 493.1. |
| | 6-chloro-3-(((R)-1-(2-cyano-7-methyl-3-((R)-2-oxa-7- | |
| | azaspiro[4.4]nonan-7-yl)quinoxalin-5- | |
| | yl)ethyl)amino)picolinic acid | |
| 255 | 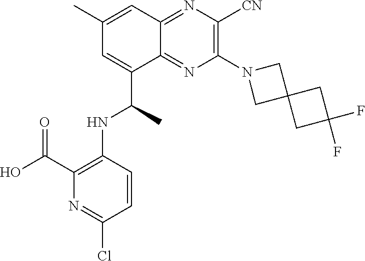 [SMILES: Cc1cc([C@@H](C)Nc2ccc(Cl)nc2C(=O)O)c2nc(N3CC4(C3)CC(F)(F)C4)c(C#N)nc2c1] | ¹H NMR (400 MHz, CD₃OD) δ = 7.59 (d, J = 1.6 Hz, 1H), 7.57 (s, 1H), 7.21 (d, J = 9.2 Hz, 1H), 7.04 (d, J = 9.2 Hz, 1H), 5.47 (q, J = 6.8 Hz, 1H), 4.62-4.57 (m, 2H), 4.55-4.50 (m, 2H), 2.91 (t, J = 12.0 Hz, 4H), 2.43 (s, 3H), 1.72 (d, J = 6.8 Hz, 3H). LCMS [M + H]⁺ = 499.3. |
| | (R)-6-chloro-3-((1-(2-cyano-3-(6,6-difluoro-2- | |
| | azaspiro[3.3]heptan-2-yl)-7-methylquinoxalin-5- | |
| | yl)ethyl)amino)picolinic acid | |
| 256 | 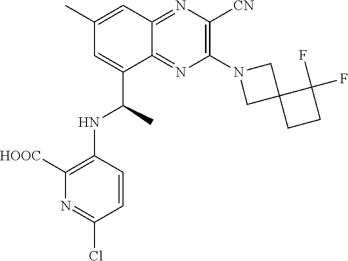 [SMILES: Cc1cc([C@@H](C)Nc2ccc(Cl)nc2C(=O)O)c2nc(N3CC4(CCC4(F)F)C3)c(C#N)nc2c1] | ¹H NMR (400 MHz, CD₃OD) δ = 7.65-7.56 (m, 2H), 7.22 (d, J = 8.8 Hz, 1H), 7.06 (d, J = 9.2 Hz, 1H), 5.52 (d, J = 6.4 Hz, 1H), 4.74 (dd, J = 9.6, 17.6 Hz, 2H), 4.49-4.34 (m, 2H), 2.61- 2.54 (m, 2H), 2.45 (s, 3H), 2.24- 2.13 (m, 2H), 1.74 (d, J = 6.8 Hz, 3H). LCMS [M + 1]⁺ = 499.1. |
| | (R)-6-chloro-3-((1-(2-cyano-3-(5,5-difluoro-2- | |
| | azaspiro[3.3]heptan-2-yl)-7-methylquinoxalin-5- | |
| | yl)ethyl)amino)picolinic acid | |
| 257A and 257B | 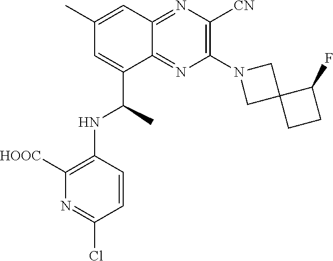 [SMILES: Cc1cc([C@@H](C)Nc2ccc(Cl)nc2C(=O)O)c2nc(N3CC4(CC[C@@H]4F)C3)c(C#N)nc2c1] | Purified by SFC prior to saponification: First eluting isomer leading to final compound 257A: ¹H NMR (400 MHz, CD₃OD) δ = 7.58 (s, 3H), 7.56 (s, 1H), 7.20 (d, J = 9.2 Hz, 1H), 7.01 (d, J = 9.2 Hz, 1H), 5.52 (q, J = 6.4 Hz, 1H), 5.11-4.90 (m, 2H), 4.44 (q, J = 9.6 Hz, 2H), 4.34 (d, J = 8.8 Hz, 1H), 2.42 (s, 3H), 2.34-2.24 (m, 1H), 2.20-1.96 (m, 2H), 1.86 (q, J = 9.6 Hz, 1H), 1.71 (d, J = 6.8 Hz, 3H). LCMS [M + Na]⁺ = 503.2. Second eluting isomer leading |
| | 6-chloro-3-(((R)-1-(2-cyano-3-((S)-5-fluoro-2- | to final compound 257B: ¹H |
| | azaspiro[3.3]heptan-2-yl)-7-methylquinoxalin-5- | NMR (400 MHz, CD₃OD) δ = |
| | yl)ethyl)amino)picolinic acid | 7.60 (d, J = 2.0 Hz, 1H), 7.55 (s, |
| | AND | 1H), 7.13 (br d, J = 9.2 Hz, 1H), |
| | 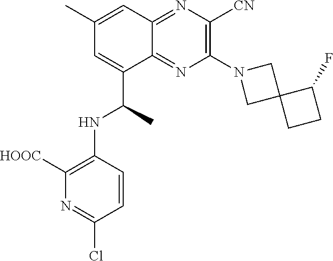 [SMILES: Cc1cc([C@@H](C)Nc2ccc(Cl)nc2C(=O)O)c2nc(N3CC4(CC[C@H]4F)C3)c(C#N)nc2c1] | 6.97 (d, J = 8.8 Hz, 1H), 5.49 (q, J = 6.8 Hz, 1H), 5.11-4.89 (m, 2H), 4.51 (d, J = 9.6 Hz, 1H), 4.39 (dd, J = 4.0, 9.2 Hz, 2H), 2.42 (s, 3H), 2.35-2.23 (m, 1H), 2.21-1.96 (m, 2H), 1.91-1.80 (m, 1H), 1.70 (d, J = 6.8 Hz, 3H). LCMS [M + Na]⁺ = 503.1. |
| | 6-chloro-3-(((R)-1-(2-cyano-3-((R)-5-fluoro-2- | |
| | azaspiro[3.3]heptan-2-yl)-7-methylquinoxalin-5- | |
| | yl)ethyl)amino)picolinic acid | |
| 258 | 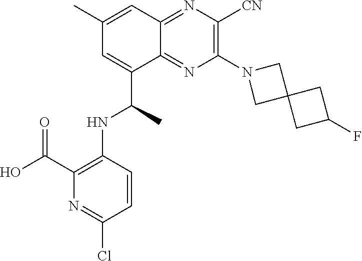 [SMILES: Cc1cc([C@@H](C)Nc2ccc(Cl)nc2C(=O)O)c2nc(N3CC4(CC(F)C4)C3)c(C#N)nc2c1] | ¹H NMR (400 MHz, CD₃OD) δ = 7.57 (d, J = 2.0 Hz, 1H), 7.53 (d, J = 0.8 Hz, 1H), 7.19 (d, J = 8.8 Hz, 1H), 7.03 (d, J = 9.2 Hz, 1H), 5.46 (q, J = 6.8 Hz, 1H), 5.12-4.93 (m, 1H), 4.54-4.39 (m, 4H), 2.80-2.68 (m, 2H), 2.56-2.44 (m, 2H), 2.41 (s, 3H), 1.70 (d, J = 6.8 Hz, 3H). LCMS [M + Na]⁺ = 503.2. |
| | (R)-6-chloro-3-((1-(2-cyano-3-(6-fluoro-2- | |
| | azaspiro[3.3]heptan-2-yl)-7-methylquinoxalin-5- | |
| | yl)ethyl)amino)picolinic acid | |
| 259 | 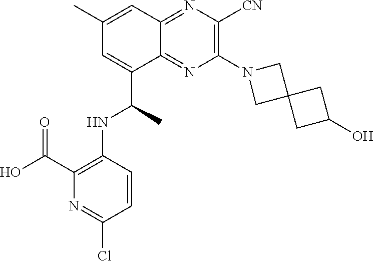 [SMILES: Cc1cc([C@@H](C)Nc2ccc(Cl)nc2C(=O)O)c2nc(N3CC4(CC(O)C4)C3)c(C#N)nc2c1] | ¹H NMR (400 MHz, CD₃OD) δ = 7.57 (d, J = 2.0 Hz, 1H), 7.55- 7.51 (m, 1H), 7.20 (d, J = 9.2 Hz, 1H), 7.03 (d, J = 9.2 Hz, 1H), 5.47 (q, J = 6.8 Hz, 1H), 4.50-4.37 (m, 4H), 4.25-4.16 (m, 1H), 2.70-2.61 (m, 2H), 2.41 (s, 3H), 2.24-2.15 (m, 2H), 1.71 (d, J = 6.8 Hz, 3H). LCMS [M + Na]⁺ = 501.2. |
| | (R)-6-chloro-3-((1-(2-cyano-3-(6-hydroxy-2- | |
| | azaspiro[3.3]heptan-2-yl)-7-methylquinoxalin-5- | |
| | yl)ethyl)amino)picolinic acid | |

### Synthesis of Examples 47A and 47B

### 6-chloro-3-(((1R)-1-(2-cyano-7-methyl-3-((1 S)-1-(trifluoromethyl)-3-azabicyclo[3.1.0]hexan-3-yl)quinoxalin-5-yl)ethyl)amino)picolinic acid and 6-chloro-3-(((1R)-1-(2-cyano-7-methyl-3-((1R)-1-(trifluoromethyl)-3-azabicyclo[3.1.0]hexan-3-yl)quinoxalin-5-yl)ethyl)amino)picolinic acid

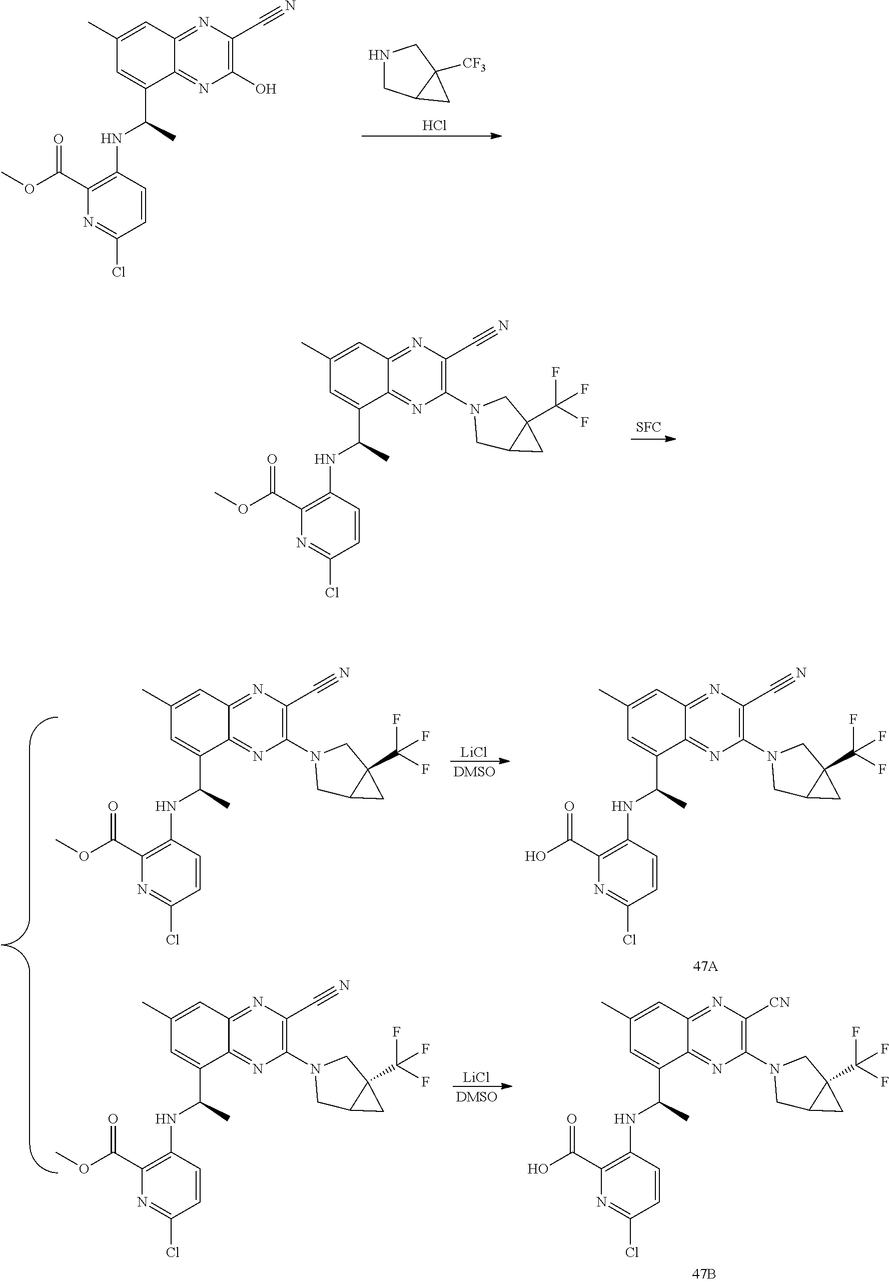

Step A: To a solution of methyl (R)-6-chloro-3-((1-(2-cyano-3-hydroxy-7-methylquinoxalin-5-yl)ethyl)amino)picolinate (180 mg, 452 μmol, 1.00 eq.) in N,N-dimethylformamide (2.00 mL) were added PyBOP (706 mg, 1.36 mmol, 3.00 eq.) and N,N-diisopropylethylamine (292 mg, 2.26 mmol, 394 μL, 5.00 eq.) and the mixture was stirred 25° C. for 30 minutes. To this mixture was added 1-(trifluoromethyl)-3-azabicyclo[3.1.0]hexane (127 mg, 679 μmol, 1.50 eq., hydrochloric acid salt) and the reaction was stirred at 25° C. for 1 hour. The mixture was then diluted with water (10.0 mL) and extracted with ethyl acetate (3×10.0 mL). The combined organic layers were washed with brine (10.0 mL), dried over anhydrous sodium sulfate, filtered, and concentrated under reduced pressure to give a residue. The residue was purified by prep-TLC (SiO₂, petroleum ether:ethyl acetate=2:1) to give methyl 6-chloro-3-(((1R)-1-(2-cyano-7-methyl-3-(1-(trifluoromethyl)-3-azabicyclo[3.1.0]hexan-3-yl)quinoxalin-5-yl)ethyl)amino)picolinate (150 mg, 250 μmol, 55.3% yield, 88.6% purity) as a yellow solid. LCMS [M+Na]⁺=553.1.

Step B: The diastereomers of methyl 6-chloro-3-(((1R)-1-(2-cyano-7-methyl-3-(1-(trifluoromethyl)-3-azabicyclo[3.1.0]hexan-3-yl)quinoxalin-5-yl)ethyl)amino)picolinate (150 mg, 88.6% purity) were separated by SFC (column: DAICEL CHIRALCEL OX (250 mm×30 mm, 10 um); mobile phase: phase A: CO₂, phase B: 0.1% NH₄OH in MeOH]; B %: 30%, isocratic elution mode). The desired fractions were concentrated to give methyl 6-chloro-3-(((1R)-1-(2-cyano-7-methyl-3-((1S)-1-(trifluoromethyl)-3-azabicyclo[3.1.0]hexan-3-yl)quinoxalin-5-yl)ethyl)amino)picolinate and 6-chloro-3-(((1R)-1-(2-cyano-7-methyl-3-((1R)-1-(trifluoromethyl)-3-azabicyclo[3.1.0]hexan-3-yl)quinoxalin-5-yl)ethyl)amino)picolinate.

First eluting isomer: White solid (50.0 mg, 84.8 μmol, 18.7% yield, 85.0% purity), LCMS [M+1]⁺=531.3 (Rt=0.692 mins).

Second eluting isomer: White solid, (65.0 mg, 110 μmol, 24.4% yield, 90.0% purity), LCMS [M+1]⁺=531.3 (Rt=0.688 mins).

Step C1: The first eluting methyl ester was saponified to the acid using the following procedure: To a solution of the first eluting methyl ester (49.0 mg, 92.3 μmol, 1.00 eq.) in DMSO (0.10 mL) was added lithium chloride (39.1 mg, 923 μmol, 18.9 μL, 10.0 eq.), and the mixture was stirred at 130° C. for 2 hours. After completion of the reaction, the mixture was cooled to 20° C., filtered, and the filtrate was purified directly by prep-HPLC (column: Phenomenex Luna C18 150×25 mm×10 um; mobile phase: phase A: 0.225% formic acid in water, phase B: acetonitrile; gradient: 75%-100% phase B) to give the desired acid (Example 43A) of the first eluting ester from the previous reaction (2.31 mg, 4.43 μmol, 4.80% yield, 99.2% purity) as a yellow solid. LCMS [M+Na]⁺=539.1.

¹H NMR (400 MHz, DMSO-d₆) δ=8.66 (br s, 1H), 7.60 (s, 2H), 7.24 (d, J=8.8 Hz, 1H), 6.98 (br d, J=8.8 Hz, 1H), 5.43 (quin, J=6.8 Hz, 1H), 4.45-4.28 (m, 2H), 4.12-3.94 (m, 2H), 2.39 (s, 3H), 2.38-2.33 (m, 1H), 1.63 (d, J=6.4 Hz, 3H), 1.43 (dd, J=5.2, 8.4 Hz, 1H), 1.08-0.98 (m, 1H).

Step C2: The second eluting methyl ester was saponified to the acid using the following procedure: To a solution of the second eluting methyl ester (64.0 mg, 121 μmol, 1.00 eq.) in DMSO (1.00 mL) was added lithium chloride (51.1 mg, 1.21 mmol, 10.0 eq.), and the mixture was stirred at 130° C. for 2 hours. After completion of the reaction, the mixture was cooled to 20° C., filtered, and the filtrate was purified directly by prep-HPLC (column: Phenomenex Luna C18 150×25 mm×10 um; mobile phase: phase A: 0.225% formic acid in water, phase B: acetonitrile; gradient: 75%-100% phase B) to give the desired acid (Example 43B) of the second eluting ester from the previous reaction (9.77 mg, 18.7 μmol, 15.5% yield, 98.7% purity) as a yellow solid. LCMS [M+Na]⁺=539.2.

¹H NMR (400 MHz, DMSO-d₆) δ=12.96 (br s, 1H), 8.46 (br d, J=7.2 Hz, 1H), 7.66-7.56 (m, 2H), 7.29 (d, J=8.8 Hz, 1H), 7.11-7.01 (m, 1H), 5.60-5.27 (m, 1H), 4.43 (d, J=10.8 Hz, 1H), 4.31 (br d, J=11.6 Hz, 1H), 4.01 (td, J=4.8, 10.4 Hz, 2H), 2.40 (s, 3H), 2.38-2.31 (m, 1H), 1.65 (d, J=6.4 Hz, 3H), 1.44 (br dd, J=6.0, 8.4 Hz, 1H), 1.04 (br t, J=5.6 Hz, 1H).

### Synthesis of Example 58

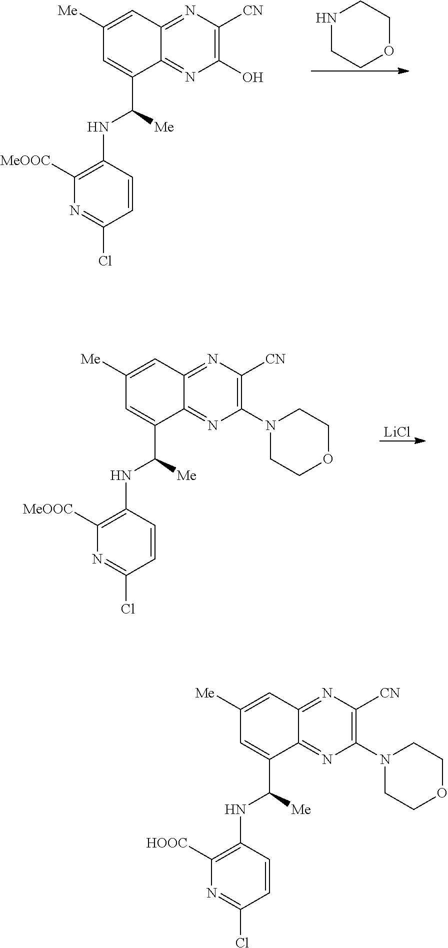

Step A: To a solution of methyl (R)-6-chloro-3-((1-(2-cyano-3-hydroxy-7-methylquinoxalin-5-yl)ethyl)amino)picolinate (50.0 mg, 113 μmol, 1.00 eq.) and morpholine (14.8 mg, 170 μmol, 14.9 μL, 1.50 eq.) in DMF (0.50 mL) was added PyBOP (88.3 mg, 170 μmol, 1.50 eq.) and N,N-diisopropylethylamine (43.9 mg, 339 μmol, 59.1 μL, 3.00 eq.), and the mixture was stirred at 25° C. for 1 hour. After this time, water (1.00 mL) was slowly added to the mixture and a yellow precipitate was formed. The suspension was then filtered, and the cake was collected and dried under vacuum to give methyl (R)-6-chloro-3-((1-(2-cyano-7-methyl-3-morpholinoquinoxalin-5-yl)ethyl)amino)picolinate (52.0 mg, 111 μmol, 98.5% yield) as a yellow solid. LCMS [M+1]⁺=467.2.

Step B: To a solution of methyl (R)-6-chloro-3-((1-(2-cyano-7-methyl-3-morpholinoquinoxalin-5-yl)ethyl)amino)picolinate (52.0 mg, 111 μmol, 1.00 eq.) in DMSO (0.50 mL) was added lithium chloride (47.2 mg, 1.11 mmol, 22.8 μL, 10.0 eq.), and the mixture was stirred at 120° C. for 6 hours. The mixture was then cooled to 25° C., filtered to give a filtrate, and the filtrate was purified by prep-HPLC (column: YMC-Actus Triart C18 150×30 mm×7 um; mobile phase: phase A: 0.225% formic acid in water, phase B: acetonitrile; gradient: 55%-85% B) to give (R)-6-chloro-3-((1-(2-cyano-7-methyl-3-morpholinoquinoxalin-5-yl)ethyl)amino)picolinic acid (8.93 mg, 19.3 μmol, 17.4% yield, 98.1% purity) as a white solid. LCMS [M+1]=453.2.

¹H NMR (400 MHz, CD₃OD) δ=7.66 (d, J=1.6 Hz, 1H), 7.63 (s, 1H), 7.19 (d, J=9.2 Hz, 1H), 7.02 (d, J=9.2 Hz, 1H), 5.53 (q, J=6.8 Hz, 1H), 3.98-3.86 (m, 4H), 3.83-3.72 (m, 4H), 2.46 (s, 3H), 1.73 (d, J=6.8 Hz, 3H).

### Synthesis of Example 70

### (R)-6-chloro-3-((1-(2-cyano-3-(3,3-difluoropyrrolidin-1-yl)-7-methylquinoxalin-5-yl)ethyl)amino)picolinic acid

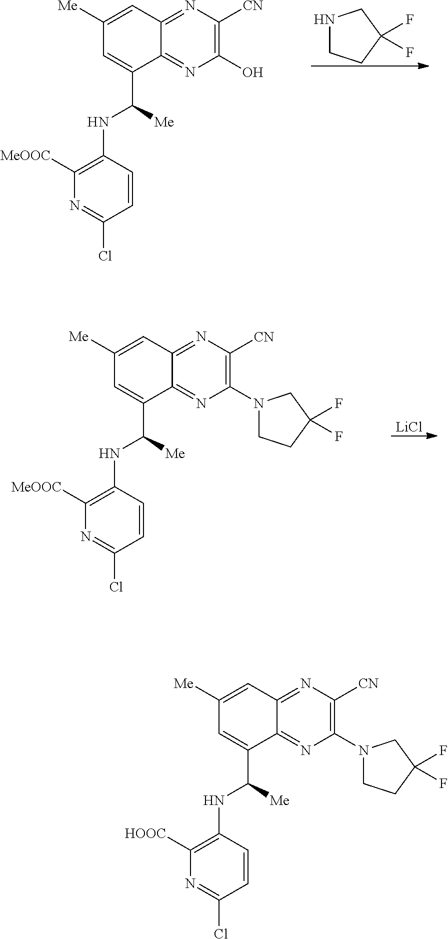

Step A: To a solution of methyl (R)-6-chloro-3-((1-(2-cyano-3-hydroxy-7-methylquinoxalin-5-yl)ethyl)amino)picolinate (70.0 mg, 158 μmol, 1.00 eq.), 3,3-difluoropyrrolidine (27.3 mg, 190 μmol, 1.20 eq., hydrochloric acid salt) in DMF (0.50 mL) was added PyBOP (124 mg, 238 μmol, 1.50 eq.) and N,N-diisopropylethylamine (143 mg, 1.11 mmol, 193 μL, 7.00 eq.), and the mixture was stirred at 25° C. for 12 hours. The mixture was then cooled to 15° C., and water (2.00 mL) was slowly added for form a yellow precipitate. The suspension was filtered, and the cake was collected and dried under vacuum to give methyl (R)-6-chloro-3-((1-(2-cyano-3-(3,3-difluoropyrrolidin-1-yl)-7-methylquinoxalin-5-yl)ethyl)amino)picolinate (70.1 mg, 144 μmol, 90.9% yield) as a yellow solid. LCMS [M+Na]⁺=509.2.

Step B: To a solution of methyl (R)-6-chloro-3-((1-(2-cyano-3-(3,3-difluoropyrrolidin-1-yl)-7-methylquinoxalin-5-yl)ethyl)amino)picolinate (70.0 mg, 144 μmol, 1.00 eq.) in DMSO (2.00 mL) was added lithium chloride (60.9 mg, 1.44 mmol, 29.5 μL, 10.0 eq.), and the mixture was stirred at 120° C. for 12 hours. The mixture was then cooled to 25° C., and filtered to collect the filtrate. The filtrate was purified via prep-HPLC (column: Welch Xtimate C18 150×25 mm×5 um; mobile phase: phase A: 0.225% formic acid in water, phase B: acetonitrile; gradient: 65%-85% B) to give (R)-6-chloro-3-((1-(2-cyano-3-(3,3-difluoropyrrolidin-1-yl)-7-methylquinoxalin-5-yl)ethyl)amino)picolinic acid (8.26 mg, 17.2 μmol, 12.0% yield, 98.7% purity) as a yellow solid. LCMS [M+Na]⁺=495.1.

¹H NMR (400 MHz, CD₃OD) δ=7.60 (s, 1H), 7.57 (s, 1H), 7.19 (d, J=9.2 Hz, 1H), 7.02 (d, J=9.2 Hz, 1H), 5.50 (q, J=6.8 Hz, 1H), 4.38-4.14 (m, 4H), 2.66-2.55 (m, 2H), 2.43 (s, 3H), 1.73 (d, J=6.8 Hz, 3H).

### Synthesis of Examples 94A and 94B

### 6-chloro-3-(((1R)-1-(2-cyano-3-((1R)-1-hydroxy-6-azaspiro[3.5]nonan-6-yl)-7-methylquinoxalin-5-yl)ethyl)amino)picolinic acid and 6-chloro-3-(((1R)-1-(2-cyano-3-((1S)-1-hydroxy-6-azaspiro[3.5]nonan-6-yl)-7-methylquinoxalin-5-yl)ethyl)amino)picolinic acid

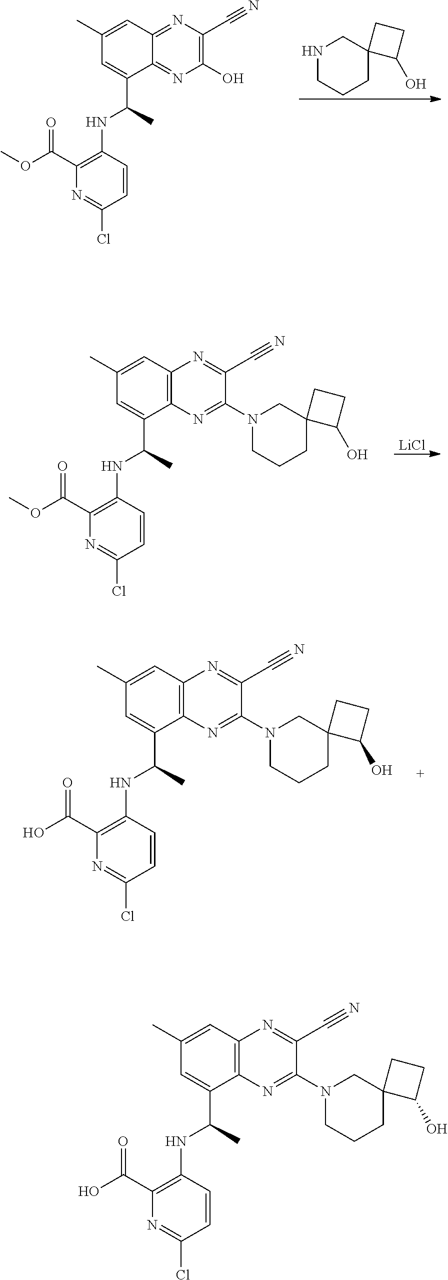

Step A: To a solution of methyl (R)-6-chloro-3-((1-(2-cyano-3-hydroxy-7-methylquinoxalin-5-yl)ethyl)amino)picolinate (80.0 mg, 201 μmol, 1.00 eq.) in DMF (1.00 mL) was added PyBOP (314 mg, 603 μmol, 3.00 eq.) and N,N-diisopropylethylamine (130 mg, 1.01 mmol, 175 μL, 5.00 eq.), and the mixture was stirred 25° C. for 30 minutes. After this time, 6-azaspiro[3.5]nonan-1-ol (42.0 mg, 302 μmol, 1.50 eq.) was added to the mixture, and the mixture was stirred at 25° C. for 1 hour. After completion of the reaction, the mixture was diluted with water (10.0 mL) and extracted with ethyl acetate (3×10.0 mL). The combined organic layers were washed with brine (10.0 mL), dried over anhydrous sodium sulfate, filtered, and concentrated under reduced pressure to give a residue. The residue was purified by prep-TLC (SiO₂, petroleum ether:ethyl acetate=2:1) to give methyl 6-chloro-3-(((1R)-1-(2-cyano-3-(1-hydroxy-6-azaspiro[3.5]nonan-6-yl)-7-methylquinoxalin-5-yl)ethyl)amino)picolinate (35.0 mg, 60.5 μmol, 30.1% yield, 94.9% purity) as a yellow solid. LCMS [M+1]⁺=521.2.

Step B: To a solution of methyl 6-chloro-3-(((1R)-1-(2-cyano-3-(1-hydroxy-6-azaspiro[3.5]nonan-6-yl)-7-methylquinoxalin-5-yl)ethyl)amino)picolinate (30.0 mg, 57.6 μmol, 1.00 eq.) in DMSO (0.50 mL) was added lithium chloride (24.4 mg, 576 μmol, 10.0 eq.), and the mixture was stirred at 130° C. for 5 hours. The mixture was then cooled to room temperature, filtered, and the filtrate was purified directly by prep-HPLC (column: Phenomenex luna C18 150×25 mm×10 um; mobile phase: phase A: 0.225% formic acid in water, phase B: acetonitrile, gradient: 55%-85% B) to the first eluting diastereomer (19.9 mg, 38.6 μmol, 67.0% yield, 98.5% purity) as a yellow solid, and the second eluting diastereomer (7.33 mg, 14.2 μmol, 24.7% yield, 98.4% purity) as a yellow solid.

Characterization data for the first eluting diastereomer: LCMS [M+1]⁺=507.2.

¹H NMR (400 MHz, DMSO-d₆) δ=8.47 (br d, J=5.2 Hz, 1H), 7.65-7.64 (m, 1H), 7.63 (br s, 1H), 7.30-7.27 (m, 1H), 7.05-6.99 (m, 1H), 5.53-5.39 (m, 1H), 4.99-4.89 (m, 1H), 3.92-3.69 (m, 4H), 3.51-3.46 (m, 1H), 2.41 (s, 3H), 2.15-2.05 (m, 1H), 1.90-1.68 (m, 5H), 1.66-1.64 (m, 3H), 1.53-1.38 (m, 2H).

Characterization data for the second eluting diastereomer: LCMS [M+1]⁺=507.2.

1H NMR (400 MHz, DMSO-d6) δ=8.45 (t, J=6.8 Hz, 1H), 7.66-7.57 (m, 2H), 7.27 (dd, J=4.8, 8.8 Hz, 1H), 6.98 (t, J=9.2 Hz, 1H), 5.55-5.44 (m, 1H), 5.06-4.85 (m, 1H), 4.21 (br dd, J=13.6, 18.8 Hz, 1H), 3.94-3.85 (m, 1H), 3.81 (t, J=7.6 Hz, 1H), 3.55 (t, J=12.4 Hz, 1H), 3.47-3.42 (m, 1H), 2.40 (s, 3H), 2.17-2.06 (m, 1H), 1.94-1.82 (m, 1H), 1.81-1.68 (m, 3H), 1.67-1.57 (m, 5H), 1.28-1.18 (m, 1H).

### Synthesis of Example 109A and 109B

### 2-(((R)-1-(2-cyano-3-((R)-4,4-difluoro-2-methylpyrrolidin-1-yl)-7-methylquinoxalin-5-yl)ethyl)amino)benzoic acid and 2-(((R)-1-(2-cyano-3-((S)-4,4-difluoro-2-methylpyrrolidin-1-yl)-7-methylquinoxalin-5-yl)ethyl)amino)benzoic acid

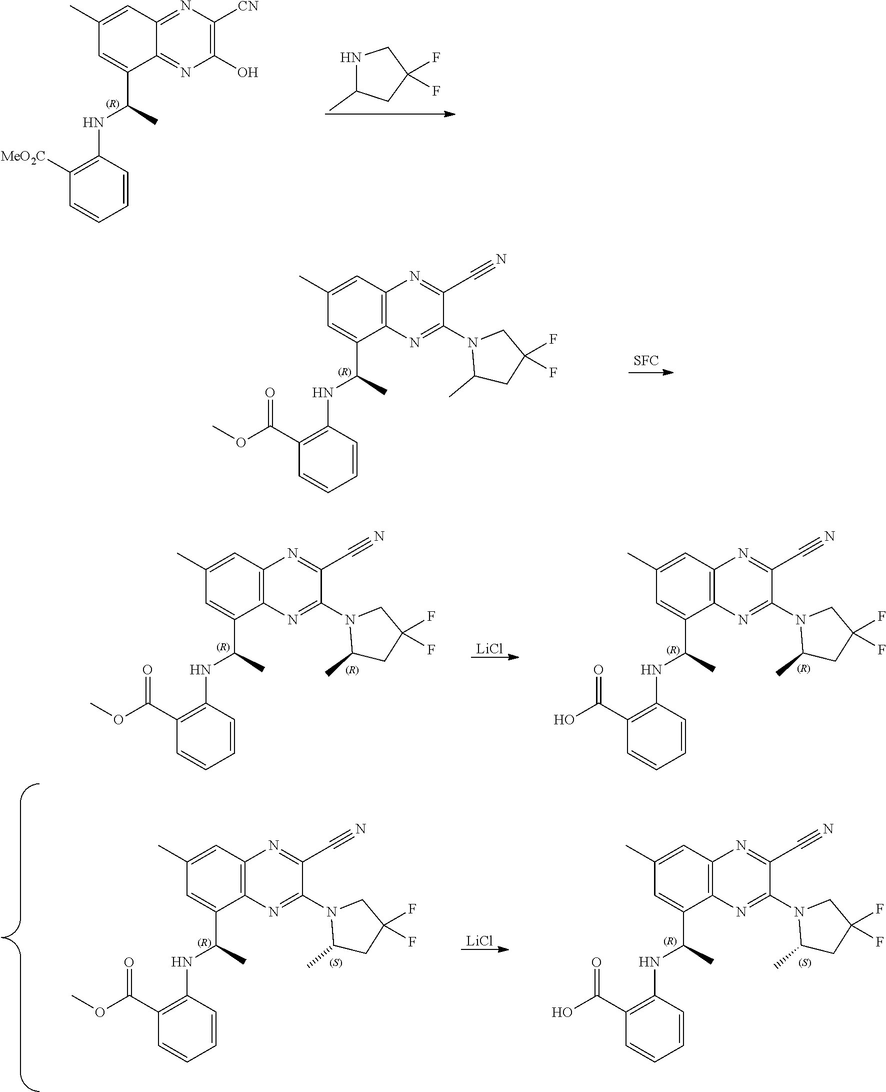

Step A: To a solution of methyl (R)-2-((1-(2-cyano-3-hydroxy-7-methylquinoxalin-5-yl)ethyl)amino)benzoate (90.0 mg, 248 μmol, 1.00 eq.) and 4,4-difluoro-2-methyl-pyrrolidine (60.2 mg, 497 μmol, 2.00 eq.) in DMF (1.00 mL) was added PyBOP (194 mg, 373 μmol, 1.50 eq.) and N,N-diisopropylethylamine (161 mg, 1.24 mmol, 216 μL, 5.00 eq.), and the mixture was stirred at 25° C. for 3 hours. The mixture was then poured into water (10.0 mL) and stirred for 2 mins. The suspension was filtered, and filter cake was dried in vacuum to give a residue. The residue was purified by SFC (column: DAICEL CHIRALCEL OX (250 mm*30 mm, 10 um); mobile phase: phase A: CO₂, phase B: 0.1% NH₄OH in MeOH; B %: 40%) to give methyl 2-(((1R)-1-(2-cyano-3-(4,4-difluoro-2-methylpyrrolidin-1-yl)-7-methylquinoxalin-5-yl)ethyl)amino)benzoate (100 mg, 214 μmol, 86.5% yield) as a yellow oil. LCMS [M+1]=466.0.

Step B: The diastereomers of methyl 2-(((1R)-1-(2-cyano-3-(4,4-difluoro-2-methylpyrrolidin-1-yl)-7-methylquinoxalin-5-yl)ethyl)amino)benzoate (100 mg, 215 μmol, 1.00 eq.) was further separated by SFC (column: DAICEL CHIRALCEL OX (250 mm*50 mm, 10 um); mobile phase: phase A: CO₂, phase B: 0.1% NH₄OH in MeOH; B %: 17%) to give the first eluting methyl ester isomer (40.0 mg, 85.9 μmol, 40.0% yield) as a yellow oil and the second eluting methyl ester isomer (45.0 mg, 96.7 μmol, 45.0% yield) as a yellow oil.

Step C1: To a solution of the first eluting methyl ester isomer from Step B (35.0 mg, 75.2 μmol, 1.00 eq.) in DMSO (2.00 mL) was added lithium chloride (31.9 mg, 752 μmol, 15.4 μL, 10.0 eq.), and the mixture was stirred at 130° C. for 8 hours. The mixture was then cooled to 25° C., further diluted with water (20.0 mL), and extracted with ethyl acetate (20.0 mL). The combined organic layers were washed with brine (20.0 mL×3), dried over sodium sulfate, filtered, and concentrated under reduced pressure to give a residue. The residue was purified by prep-HPLC (YMC-Actus Triart C18 150×30 mm×7 um; mobile phase: phase A: 0.225% formic acid in water, phase B: acetonitrile; gradient: 60%-90%) to give 2-(((R)-1-(2-cyano-3-((R)-4,4-difluoro-2-methylpyrrolidin-1-yl)-7-methylquinoxalin-5-yl)ethyl)amino)benzoic acid OR 2-(((R)-1-(2-cyano-3-((S)-4,4-difluoro-2-methylpyrrolidin-1-yl)-7-methylquinoxalin-5-yl)ethyl)amino)benzoic acid (4.07 mg, 8.73 μmol, 11.6% yield, 96.8% purity) as a yellow solid. LCMS [M+1]=452.2.

¹H NMR (400 MHz, CD₃OD) δ=7.88 (dd, J=1.6, 8.0 Hz, 1H), 7.63 (d, J=1.6 Hz, 1H), 7.58 (s, 1H), 7.12-7.06 (m, 1H), 6.49 (t, J=7.6 Hz, 1H), 6.36 (d, J=8.4 Hz, 1H), 5.56 (q, J=6.8 Hz, 1H), 5.09-4.99 (m, 1H), 4.54-4.41 (m, 1H), 4.30 (q, J=12.0 Hz, 1H), 2.84-2.72 (m, 1H), 2.42 (s, 3H), 2.39-2.26 (m, 1H), 1.68 (d, J=6.8 Hz, 3H), 1.51 (d, J=6.8 Hz, 3H).

Step C1: To a solution of the second eluting methyl ester isomer from Step B (40.0 mg, 85.9 μmol, 1.00 eq.) in DMSO (1.00 mL) was added lithium chloride (36.4 mg, 859 μmol, 17.6 μL, 10.0 eq.), and the mixture was stirred at 130° C. for 8 hours. The mixture was then cooled to 25° C., further diluted with water (20.0 mL), and extracted with ethyl acetate (20.0 mL). The combined organic layers were washed with brine (20.0 mL×3), dried over sodium sulfate, filtered, and concentrated under reduced pressure to give a residue. The residue was purified by prep-HPLC (column: YMC-Actus Triart C18 150×30 mm×7 um; mobile phase: phase A: 0.225% formic acid in water, phase B: acetonitrile; gradient: 60%-90% B) to give 2-(((R)-1-(2-cyano-3-((R)-4,4-difluoro-2-methylpyrrolidin-1-yl)-7-methylquinoxalin-5-yl)ethyl)amino)benzoic acid OR 2-(((R)-1-(2-cyano-3-((S)-4,4-difluoro-2-methylpyrrolidin-1-yl)-7-methylquinoxalin-5-yl)ethyl)amino)benzoic acid (6.10 mg, 13.1 μmol, 15.3% yield, 97% purity) as a yellow solid. LCMS [M+1]=452.2.

¹H NMR (400 MHz, CD₃OD) δ=7.89 (d, J=8.0 Hz, 1H), 7.61 (s, 1H), 7.58 (br s, 1H), 7.10-7.03 (m, 1H), 6.49 (t, J=7.6 Hz, 1H), 6.33 (d, J=8.4 Hz, 1H), 5.60 (q, J=6.8 Hz, 1H), 4.98-4.90 (m, 1H), 4.46 (q, J=13.2 Hz, 1H), 4.31 (q, J=12.0 Hz, 1H), 2.83-2.68 (m, 1H), 2.41 (s, 3H), 2.38-2.26 (m, 1H), 1.61 (d, J=6.8 Hz, 3H), 1.50 (d, J=6.0 Hz, 3H).

### Example 110A and 110B

### 6-chloro-3-(((R)-1-(2-cyano-3-((R)-3-methoxypyrrolidin-1-yl)-7-methylquinoxalin-5-yl)ethyl)amino)picolinic acid and 6-chloro-3-(((R)-1-(2-cyano-3-((S)-3-methoxypyrrolidin-1-yl)-7-methylquinoxalin-5-yl)ethyl)amino)picolinic acid

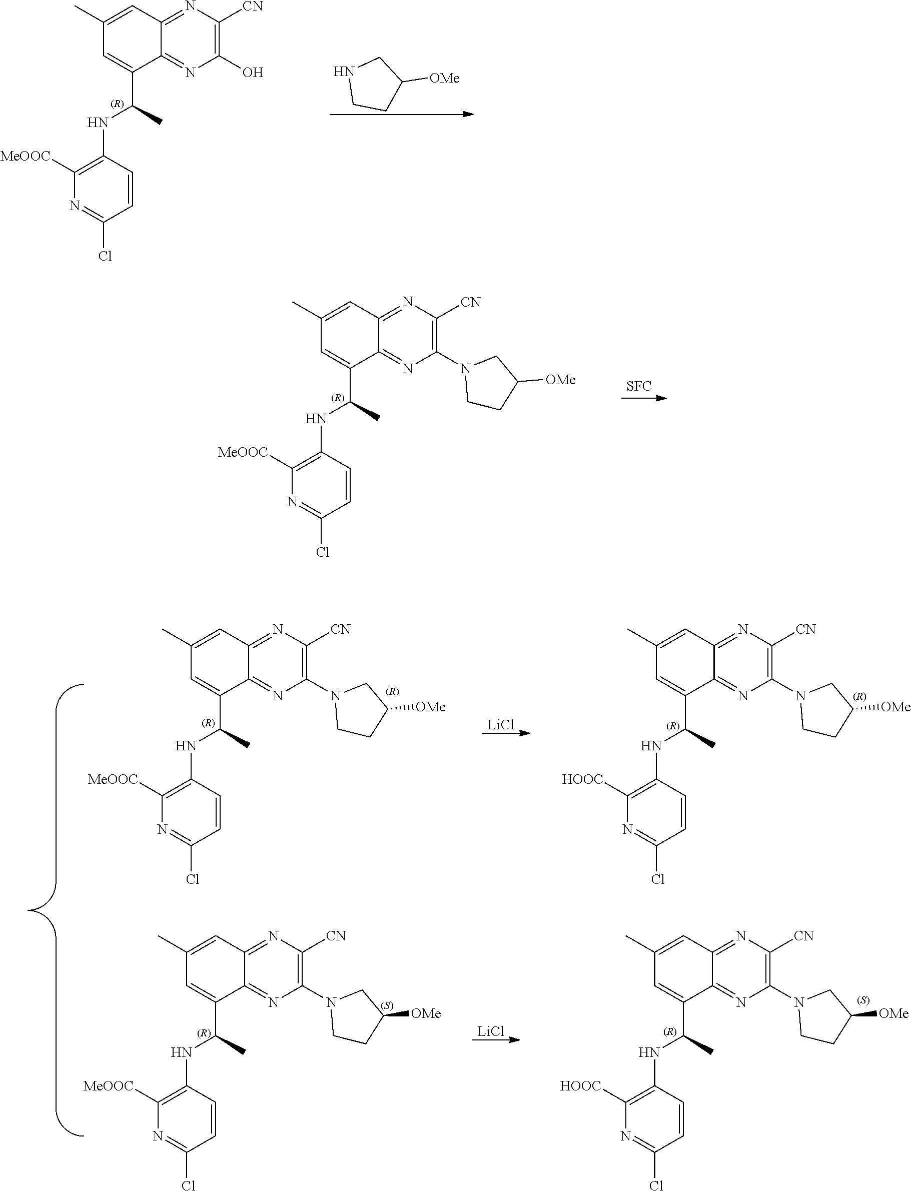

Step A: To a solution of methyl (R)-6-chloro-3-((1-(2-cyano-3-hydroxy-7-methylquinoxalin-5-yl)ethyl)amino)picolinate (70.0 mg, 176 μmol, 1.00 eq.), 3-methoxypyrrolidine (35.6 mg, 352 μmol, 2.00 eq.) in DMF (1.00 mL) was added PyBOP (137 mg, 264 μmol, 1.50 eq.) and N,N-diisopropylethylamine (159 mg, 1.23 mmol, 215 μL, 7.00 eq.), and the mixture was stirred at 30° C. for 2 hours. The mixture was then poured into water (10.0 mL) and stirred for 2 mins. The resulting suspension was filtered, and the filter cake was dried in vacuum to give methyl 6-chloro-3-(((1R)-1-(2-cyano-3-(3-methoxypyrrolidin-1-yl)-7-methylquinoxalin-5-yl)ethyl)amino)picolinate (80.0 mg, 166 μmol, 94.5% yield) as a yellow oil. LCMS [M+Na]⁺=503.1.

Step B: The methyl 6-chloro-3-(((1R)-1-(2-cyano-3-(3-methoxypyrrolidin-1-yl)-7-methylquinoxalin-5-yl)ethyl)amino)picolinate (80.0 mg, 166 μmol, 1.00 eq.) was purified by SFC (column: Welch Ultimate XB—CN 250×50×10 um; mobile phase: phase A: Hexanes, phase B: 0.1% NH₄OH in EtOH; gradient: 1%-40% B) to give the first eluting methyl ester isomer (40.0 mg, 83.2 μmol, 50.0% yield) as a yellow oil and the second eluting methyl ester isomer (45.0 mg, 93.6 μmol, 56.3% yield) as a yellow oil.

Step C1: To a solution of the first eluting methyl ester isomer from Step B (40.0 mg, 83.2 μmol, 1.00 eq.) in DMSO (1.00 mL) was added lithium chloride (35.3 mg, 832 μmol, 17.1 μL, 10.0 eq.), and the mixture was stirred at 130° C. for 2 hours. The reaction was then cooled to 25° C. and adjusted to pH=6 with hydrochloric acid (2M in water, 2.00 mL), and further diluted with water (20.0 mL). The resulting aqueous solution was extracted with ethyl acetate (20.0 mL), and the combined organic layers were washed with brine (20.0 mL×3), dried over sodium sulfate, filtered, and concentrated under reduced pressure to give a residue. The residue was purified by prep-HPLC (column: Phenomenex luna C18 150×25 mm×10 um; mobile phase: phase A: 0.225% formic acid in water, phase B: acetonitrile; gradient: 60%-90% B) to give 6-chloro-3-(((R)-1-(2-cyano-3-((R)-3-methoxypyrrolidin-1-yl)-7-methylquinoxalin-5-yl)ethyl)amino)picolinic acid OR 6-chloro-3-(((R)-1-(2-cyano-3-((S)-3-methoxypyrrolidin-1-yl)-7-methylquinoxalin-5-yl)ethyl)amino)picolinic acid (2.23 mg, 4.39 μmol, 5.28% yield, 91.9% purity) as a yellow solid. LCMS [M+1]=467.2.

¹H NMR (400 MHz, CD₃OD) δ=7.55 (s, 1H), 7.53 (s, 1H) 7.20 (d, J=9.2 Hz, 1H), 7.02 (d, J=8.8 Hz, 1H), 5.58-5.48 (m, 1H), 5.52 (q, J=6.4 Hz, 1H), 4.24-4.16 (m, 1H), 4.08-3.92 (m, 4H), 3.41 (s, 3H), 2.41 (s, 3H), 2.36-2.08 (m, 2H), 1.72 (d, J=6.8 Hz, 3H).

Step C2: To a solution of the second eluting methyl ester isomer from Step B (10.0 mg, 20.8 μmol, 1.00 eq.) in DMSO (1.00 mL) was added lithium chloride (8.81 mg, 208 μmol, 4.26 μL, 10.0 eq.), and the mixture was stirred at 130° C. for 2 hours. The mixture was then cooled to 25° C. and adjusted to pH=6 with hydrochloric acid (2M in water, 2.00 mL), further diluted with water (20.0 mL), and the resulting aqueous solution was extracted with ethyl acetate (20.0 mL). The combined organic layers were washed with brine (20.0 mL×3), dried over sodium sulfate, filtered, and concentrated under reduced pressure to give a residue. The residue was purified by prep-HPLC (column: Phenomenex Luna C18 150×25 mm×10 um; mobile phase: phase A: 0.225% formic acid in water, phase B: acetonitrile; gradient: 60%-90% B) to 6-chloro-3-(((R)-1-(2-cyano-3-((R)-3-methoxypyrrolidin-1-yl)-7-methylquinoxalin-5-yl)ethyl)amino)picolinic acid OR 6-chloro-3-(((R)-1-(2-cyano-3-((S)-3-methoxypyrrolidin-1-yl)-7-methylquinoxalin-5-yl)ethyl)amino)picolinic acid (1.99 mg, 4.18 μmol, 20.1% yield, 98.1% purity) as a yellow solid. LCMS [M+1]⁺=467.3.

¹H NMR (400 MHz, methanol-d₄) δ=7.56 (s, 1H), 7.54 (s, 1H), 7.20 (d, J=8.8 Hz, 1H), 7.02 (d, J=9.2 Hz, 1H), 5.51 (q, J=6.8 Hz, 1H), 4.23-4.17 (m, 1H), 4.07-3.92 (m, 4H), 3.42 (s, 3H), 2.42 (s, 3H), 2.33-2.23 (m, 1H), 2.21-2.08 (m, 1H), 1.72 (d, J=6.8 Hz, 3H).

### Example 260

### 6-chloro-3-(((R)-1-(2-cyano-7-methyl-3-(( )-2-methylmorpholino)quinoxalin-5-yl)ethyl)amino)picolinic acid

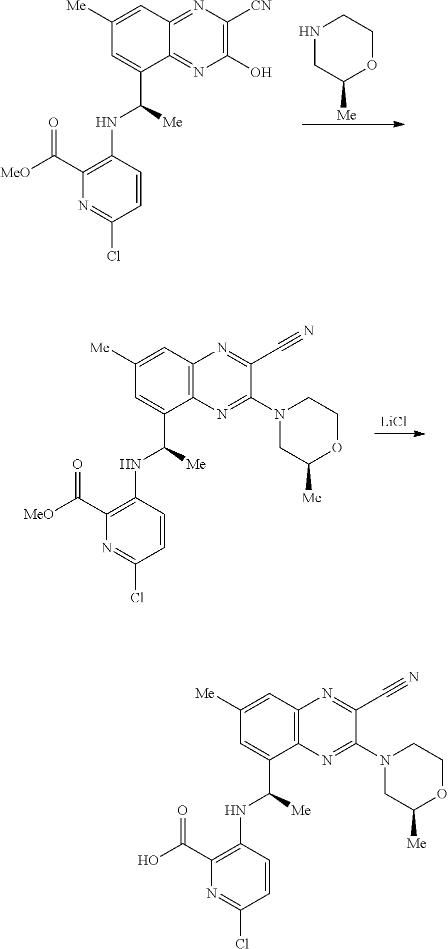

Step A: To a solution of methyl methyl (R)-6-chloro-3-((1-(2-cyano-3-hydroxy-7-methylquinoxalin-5-yl)ethyl)amino)picolinate (40.0 mg, 101 μmol, trifluoroacetic acid salt) in N, N-dimethylformamide (0.50 mL) were added PyBOP (78.5 mg, 151 μmol, 1.50 eq.) and N,N-diisopropylethylamine (65.0 mg, 503 μmol, 87.6 μL, 3.00 eq.), and the mixture was stirred at 25° C. for 30 minutes. After this time, (S)-2-methylmorpholine (12.2 mg, 121 μmol, 1.50 eq.) was added to the above mixture at 25° C. and the mixture was left to stir at 25° C. for 2.5 hours. After completion of the reaction, the mixture was diluted with water (5.00 mL) and extracted with ethyl acetate (5.00 mL×3). The combined organic layers were washed with brine (5.00 mL×3), dried over anhydrous sodium sulfate, filtered, and concentrated under reduced pressure to give a residue. The residue was purified by prep-TLC (SiO2, petroleum ether/ethyl acetate=3/1) to give methyl 6-chloro-3-(((R)-1-(2-cyano-7-methyl-3-((S)-2-methylmorpholino)quinoxalin-5-yl)ethyl)amino)picolinate (25.0 mg, 52.0 μmol, 51.7% yield) as yellow solid. LCMS [M+1]⁺=481.2.

Step B: To a solution of methyl 6-chloro-3-(((R)-1-(2-cyano-7-methyl-3-((S)-2-methylmorpholino)quinoxalin-5-yl)ethyl)amino)picolinate (25.0 mg, 52.0 μmol, μmol, 1.00 eq.) in DMSO (0.80 mL) was added lithium chloride (22.0 mg, 520 μmol 10.0 eq.), and the reaction was stirred at 120° C. for 5 hours. After completion of the reaction, the reaction mixture was cooled to 20° C. and filtered, and the filter liquor was purified by prep-HPLC (column: Phenomenex luna C18 150×25 mm×10 um; mobile phase: phase A: 0.225% formic acid in water, phase B: acetonitrile; gradient: 55%-85% B) to give 6-chloro-3-(((R)-1-(2-cyano-7-methyl-3-((S)-2-methylmorpholino)quinoxalin-5-yl)ethyl)amino)picolinic acid (5.95 mg, 12.7 μmol, 24.4% yield, 99.7% purity) as yellow solid. LCMS [M+1]⁺=467.1.

1H NMR (400 MHz, CD₃OD) δ=7.66 (s, 2H), 7.21 (d, J=8.8 Hz, 1H), 7.04 (d, J=8.8 Hz, 1H), 5.53 (q, J=6.8 Hz, 1H), 4.29-4.19 (m, 2H), 4.05 (dd, J=2.0, 11.6 Hz, 1H), 3.91-3.81 (m, 2H), 3.28-3.22 (m, 1H), 2.97 (dd, J=10.0, 12.8 Hz, 1H), 2.47 (s, 3H), 1.73 (d, J=6.8 Hz, 3H), 1.26 (d, J=6.0 Hz, 3H).

### Examples 261A, 261B, 261C, and 261D

### 6-chloro-3-(((R)-1-(2-cyano-3-((S)-3-((S)-1-hydroxyethyl)piperidin-1-yl)-7-methylquinoxalin-5-yl)ethyl)amino)picolinic acid, 6-chloro-3-(((R)-1-(2-cyano-3-((S)-3-((R)-1-hydroxyethyl)piperidin-1-yl)-7-methylquinoxalin-5-yl)ethyl)amino)picolinic acid, 6-chloro-3-(((R)-1-(2-cyano-3-((R)-3-((S)-1-hydroxyethyl)piperidin-1-yl)-7-methylquinoxalin-5-yl)ethyl)amino)picolinic acid, and 6-chloro-3-(((R)-1-(2-cyano-3-((R)-3-((R)-1-hydroxyethyl)piperidin-1-yl)-7-methylquinoxalin-5-yl)ethyl)amino)picolinic acid

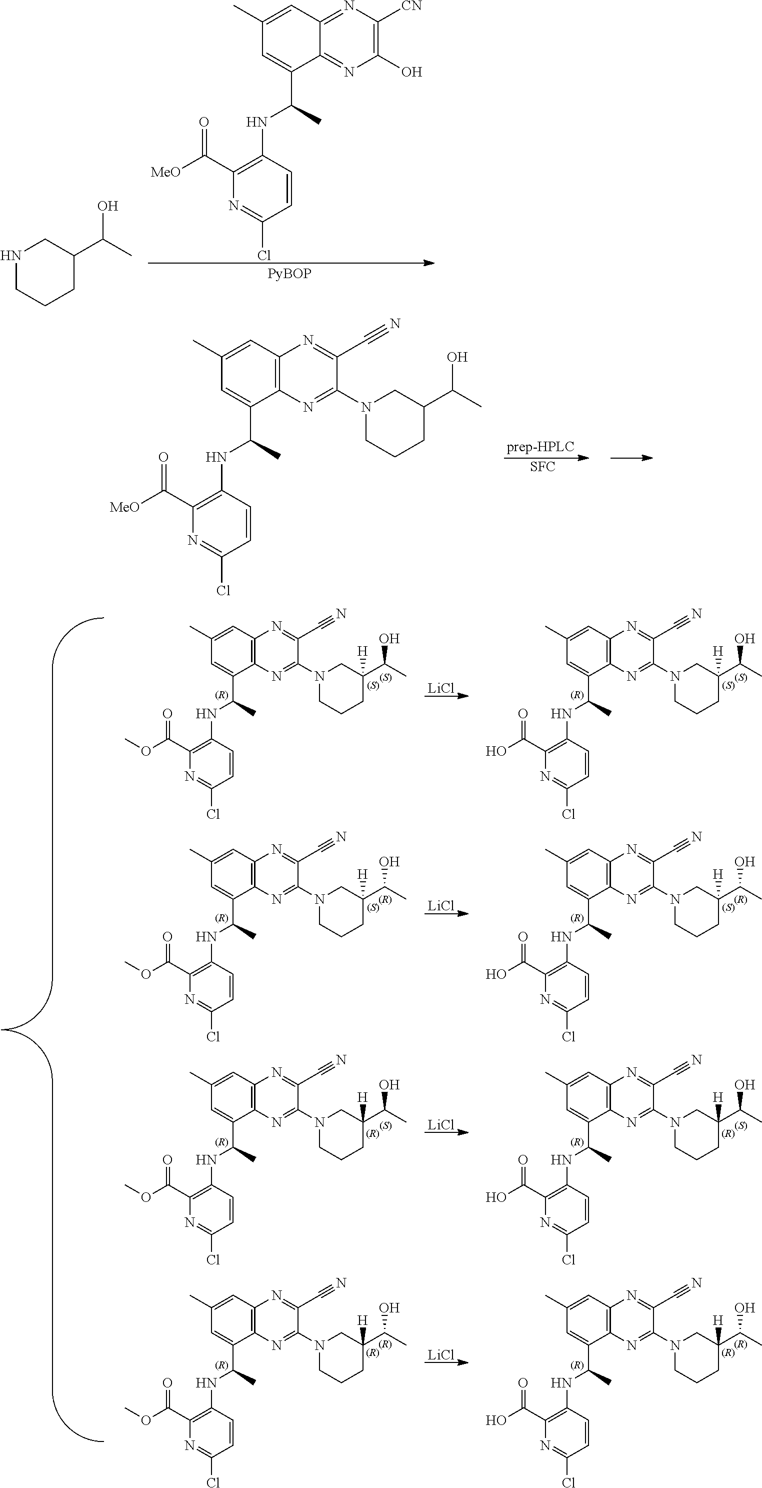

To a solution of methyl (R)-6-chloro-3-((1-(2-cyano-3-hydroxy-7-methylquinoxalin-5-yl)ethyl)amino)picolinate (600 mg, 1.51 mmol, 1.00 eq.), N,N-diisopropylethylamine (975 mg, 7.54 mmol, 1.31 mL, 5.00 eq.) in N,N-dimethylformamide (6.00 mL) was added PyBOP (1.18 g, 2.26 mmol, 1.50 eq.), and the mixture was stirred at 25° C. for 1 hour. After this time 1-(piperidin-3-yl) ethan-1-ol (292 mg, 2.26 mmol, 1.50 eq.) was added to the mixture at 25° C., and the reaction was further stirred at 25° C. for 12 hours. After completion of the reaction, the mixture was diluted with water (100 mL) and extracted with ethyl acetate (3× 100 mL). The combined organic layers were washed with brine (100 mL), dried over anhydrous sodium sulfate, filtered, and concentrated under reduced pressure to give a residue. The residue was purified by column chromatography (SiO₂, petroleum ether:ethyl acetate=1:0 to 3:1) to give a crude product. The crude product was then purified by prep-HPLC (column: Phenomenex luna C18 150×40 mm×15 um; mobile phase: phase A: 0.225% formic acid in water, phase B: acetonitrile; gradient: 60%-90% B) to give the first eluting mixture of the methyl esters of 261A and 261D (100 mg, 173 μmol, 11.5% yield) as a yellow solid and a second peak. The second peak was purified by prep-HPLC (column: YMC-Actus Triart C18 150×30 mm×7 um; mobile phase: phase A: 0.225% formic acid in water, phase B: acetonitrile; gradient: 63%-93% B) to give the second eluting mixture of the methyl ester of 261B and 261C (90.0 mg, 172 μmol, 11.4% yield) as a yellow solid. LCMS [M+1]⁺=509.2.

The first eluting mixture of the methyl esters of 261A and 261D (100 mg, 173 μmol, 11.5% yield) was separated by SFC (column: DAICEL CHIRALPAK IC (250 mm×30 mm, 10 um); mobile phase: phase A: CO₂, phase B: i-PrOH (0.1% NH₃H₂O); B %: 55%, isocratic elution). The desired fractions were collected and concentrated under reduce pressure to give the methyl ester of 261A (25.0 mg, 49.1 μmol, 3.26% yield) as a white solid and the methyl ester of 261B (43.0 mg, 82.0 μmol, 5.43% yield) as a yellow solid.

Methyl ester of 261A: LCMS [M+Na]⁺=531.2.

Methyl ester of 261D: LCMS [M+Na]⁺=531.2.

The second eluting mixture of the methyl esters of 261B and 261C (90.0 mg, 172 μmol, 11.4% yield) was separated by SFC (column: DAICEL CHIRALPAK IC (250 mm×30 mm, 10 um); mobile phase: phase A: CO2, phase B: i-PrOH (0.1% NH₃H₂O); B %: 55%, isocratic elution). The desired fractions were collected and concentrated under reduce pressure to give the methyl ester of 261B (22.0 mg, 43.2 μmol, 2.87% yield) as a white solid and the methyl ester of 261C (45.0 mg, 88.4 μmol, 5.86% yield) as a white solid.

Methyl ester of 261B: LCMS [M+Na]⁺=531.2.

Methyl ester of 261C: LCMS [M+Na]⁺=531.2.

To a solution of methyl ester of 261A (24.0 mg, 47.1 μmol, 1.00 eq.) in DMSO (0.500 mL) was added lithium chloride (20.0 mg, 472 μmol, 9.67 μL, 10.0 eq.). The reaction was stirred at 130° C. for 4 hours. After completion of the reaction, the mixture was cooled to 20° C. Then the mixture was filtered and the filtrate was purified directly by prep-HPLC (column: Phenomenex luna C18 150×25 mm×10 um; mobile phase: phase A: 0.225% formic acid in water, phase B: acetonitrile; gradient: 55%-85% B) to give 261A (11.3 mg, 22.6 μmol, 48.0% yield) as a yellow solid.

¹H NMR (400 MHz, DMSO-d₆) δ=12.99 (s, 1H), 8.47 (br d, J=6.4 Hz, 1H), 7.79-7.50 (m, 2H), 7.29 (d, J=8.8 Hz, 1H), 6.99 (d, J=9.2 Hz, 1H), 5.46 (t, J=6.8 Hz, 1H), 4.64-4.41 (m, 1H), 4.33 (br d, J=12.8 Hz, 2H), 3.53 (br t, J=6.4 Hz, 1H), 3.08 (br t, J=11.6 Hz, 1H), 2.96-2.83 (m, 1H), 2.41 (s, 3H), 1.98 (br d, J=10.0 Hz, 1H), 1.90-1.77 (m, 1H), 1.70-1.54 (m, 5H), 1.41-1.27 (m, 1H), 1.12 (d, J=6.4 Hz, 3H).

LCMS [M+Na]⁺=517.4.

To a solution of the methyl ester of 261B (22.0 mg, 43.2 μmol, 1.00 eq.) in DMSO (0.500 mL) was added lithium chloride (18.3 mg, 432 μmol, 8.86 μL, 10.0 eq.). The reaction was stirred at 130° C. for 4 hours. After completion of the reaction, the mixture was cooled to 20° C. Then the mixture was filtered and the filtrate was purified directly by prep-HPLC (column: Phenomenex luna C18 150×25 mm×10 um; mobile phase: phase A: 0.225% formic acid in water, phase B: acetonitrile; gradient: 58%-88% B) to 261B (13.5 mg, 83.0 μmol, 61.4% yield) as a yellow solid.

¹H NMR (400 MHz, DMSO-d₆) δ=13.00 (br s, 1H), 8.46 (br d, J=7.2 Hz, 1H), 7.64 (d, J=6.0 Hz, 2H), 7.29 (d, J=8.8 Hz, 1H), 7.02 (d, J=8.8 Hz, 1H), 5.50 (br t, J=6.8 Hz, 1H), 4.52 (br d, J=11.6 Hz, 2H), 4.27 (br d, J=12.0 Hz, 1H), 3.54-3.43 (m, 1H), 3.12 (br t, J=11.6 Hz, 1H), 2.99 (dd, J=10.4, 13.2 Hz, 1H), 2.41 (s, 3H), 1.88-1.73 (m, 2H), 1.71-1.55 (m, 5H), 1.42-1.29 (m, 1H), 1.10 (d, J=6.4 Hz, 3H).

LCMS [M+Na]⁺=517.4.

To a solution of the methyl ester of 261C (45.0 mg, 88.4 μmol, 1.00 eq.) in DMSO (0.500 mL) was added lithium chloride (37.5 mg, 884 μmol, 18.1 μL, 10.0 eq.). The reaction was stirred at 130° C. for 4 hours. After completion of the reaction, the mixture was cooled to 20° C. Then the mixture was filtered and the filtrate was purified directly by prep-HPLC (column: C18 150×30 mm; mobile phase: phase A: 0.225% formic acid in water, phase B: acetonitrile; gradient: 62%-92% B) to 261C (12.6 mg, 25.3 μmol, 28.6% yield) as a yellow solid.

¹H NMR (400 MHz, DMSO-d₆) δ=13.01 (br s, 1H), 8.45 (d, J=7.2 Hz, 1H), 7.67-7.58 (m, 2H), 7.29 (d, J=8.8 Hz, 1H), 7.00 (d, J=9.2 Hz, 1H), 5.50 (quin, J=6.8 Hz, 1H), 4.52 (br d, J=12.4 Hz, 2H), 4.26 (br d, J=13.2 Hz, 1H), 3.48 (br t, J=6.4 Hz, 1H), 3.19-3.11 (m, 1H), 2.98 (dd, J=10.4, 13.2 Hz, 1H), 2.41 (s, 3H), 1.87-1.71 (m, 2H), 1.69-1.57 (m, 5H), 1.43-1.30 (m, 1H), 1.11 (d, J=6.4 Hz, 3H).

LCMS [M+Na]⁺=517.4.

To a solution of methyl 6-chloro-3-(((R)-1-(2-cyano-3-((R)-3-((R)-1-hydroxyethyl)piperidin-1-yl)-7-methylquinoxalin-5-yl)ethyl)amino)picolinate (35.0 mg, 68.8 μmol, 1.00 eq.) in DMSO (0.500 mL) was added lithium chloride (29.2 mg, 688 μmol, 14.1 μL, 10.0 eq.). The reaction was stirred at 130° C. for 4 hours. After completion of the reaction, the mixture was cooled to 20° C. Then the mixture was filtered and the filtrate was purified directly by prep-HPLC (column: Phenomenex luna C18 150×25 mm×10 um; mobile phase: phase A: 0.225% formic acid in water, phase B: acetonitrile; gradient: 58%-88% B) to give 261D (8.73 mg, 17.5 μmol, 25.4% yield) as a yellow solid.

¹H NMR (400 MHz, DMSO-d₆) δ=13.04 (s, 1H), 8.49 (br d, J=6.4 Hz, 1H), 7.71-7.60 (m, 2H), 7.30 (d, J=8.8 Hz, 1H), 7.01 (d, J=8.8 Hz, 1H), 5.47 (t, J=6.8 Hz, 1H), 4.61-4.45 (m, 1H), 4.41-4.26 (m, 2H), 3.54-3.47 (m, 1H), 3.11-3.01 (m, 1H), 2.93 (dd, J=11.6, 12.8 Hz, 1H), 2.41 (s, 3H), 2.03-1.95 (m, 1H), 1.89-1.78 (m, 1H), 1.70-1.55 (m, 5H), 1.40-1.21 (m, 1H), 1.10 (d, J=6.4 Hz, 3H).

LCMS [M+Na]⁺=517.4.

### Examples 262-572

The compounds in Table 2 are prepared essentially according to the procedures set forth in the above schemes and examples.

TABLE 2

| Example | | |
|---|---|---|
| No. | Name | Structure |
| 262 | (R)-2-((1-(3-(4-(bicyclo[1.1.1]pentan- 1-yl-3-d)piperidin-1-yl)-2-cyano-7- methylquinoxalin-5-yl)ethyl)amino)- benzoic acid | 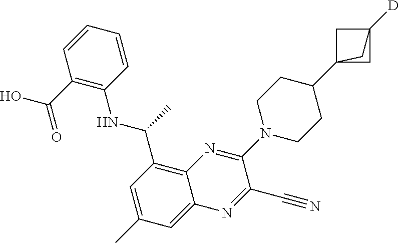 [SMILES: [2H]C12CC(C3CCN(c4nc5c([C@@H](C)Nc6ccccc6C(=O)O)cc(C)cc5nc4C#N)CC3)(C1)C2] |
| 263 | (R)-2-((1-(2-cyano-3-((3-cyanophen- yl)amino)-7-methylquinoxalin-5-yl)- ethyl)amino)benzoic acid | 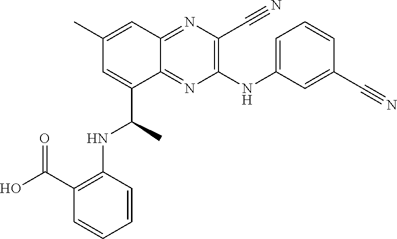 [SMILES: Cc1cc([C@@H](C)Nc2ccccc2C(=O)O)c2nc(Nc3cccc(C#N)c3)c(C#N)nc2c1] |
| 264 | 2-(((1R)-1-(2-cyano-7-methyl-3- (((tetrahydrofuran-3-yl)methyl)- amino)quinoxalin-5-yl)ethyl)- amino)benzoic acid | 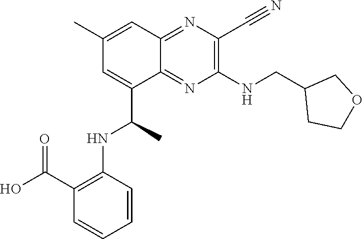 [SMILES: Cc1cc([C@@H](C)Nc2ccccc2C(=O)O)c2nc(NCC3CCOC3)c(C#N)nc2c1] |
| 265 | (R)-2-((1-(2-cyano-7-methyl-3- ((oxetan-3-ylmethyl)amino)quinox- alin-5-yl)ethyl)amino)benzoic acid | 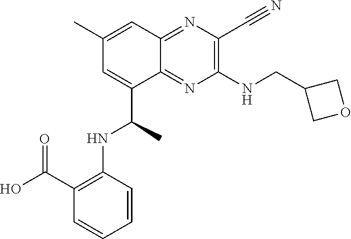 [SMILES: Cc1cc([C@@H](C)Nc2ccccc2C(=O)O)c2nc(NCC3COC3)c(C#N)nc2c1] |
| 266 | (R)-2-((1-(2-cyano-3-((2-cyanoallyl)- amino)-7-methylquinoxalin-5-yl)- ethyl)-amino)benzoic acid | 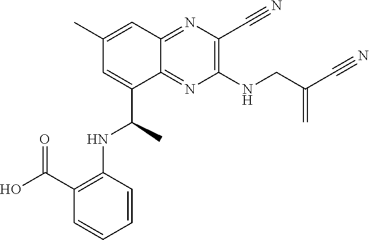 [SMILES: C=C(C#N)CNc1nc2c([C@@H](C)Nc3ccccc3C(=O)O)cc(C)cc2nc1C#N] |
| 267 | (R)-2-((1-(2-cyano-3-cyclobutyl- amino)-7-methylquinoxalin-5-yl)- ethyl)amino)benzoic acid | 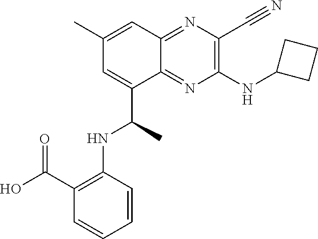 [SMILES: Cc1cc([C@@H](C)Nc2ccccc2C(=O)O)c2nc(NC3CCC3)c(C#N)nc2c1] |
| 268 | (R)-2-((1-(2-cyano-3-((3-(hydroxy- methyl)bicyclo[1.1.1]pentan-1-yl)- amino)-7-methylquinoxalin-5-yl)- ethyl)amino)benzoic acid | 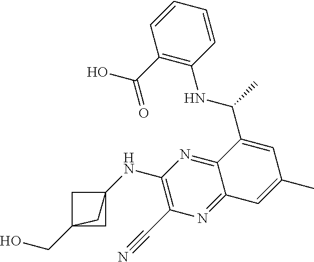 [SMILES: Cc1cc([C@@H](C)Nc2ccccc2C(=O)O)c2nc(NC34CC(CO)(C3)C4)c(C#N)nc2c1] |
| 269 | (R)-2-((1-(2-cyano-3-(5,6-dihydro- imidazo[1,2-a]pyrazin-7(8H)-yl)-7- methylquinoxalin-5-yl)ethyl)amino)- benzoic acid | 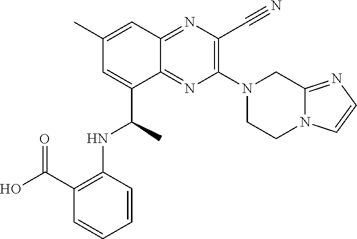 [SMILES: Cc1cc([C@@H](C)Nc2ccccc2C(=O)O)c2nc(N3CCn4ccnc4C3)c(C#N)nc2c1] |
| 270 | (R)-2-((1-(2-cyano-3-(2,3-dimethyl- 5,6-dihydroimidazo[1,2-a]pyrazin- 7(8H)-yl)-7-methylquinoxalin-5-yl)- ethyl)amino)benzoic acid | 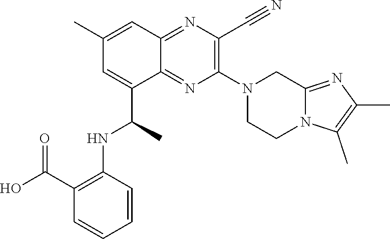 [SMILES: Cc1cc([C@@H](C)Nc2ccccc2C(=O)O)c2nc(N3CCn4c(nc(C)c4C)C3)c(C#N)nc2c1] |
| 271 | (R)-2-((1-(2-cyano-3-(5,6-dihydro- imidazo[1,5-a]pyrazin-7(8H)-yl)-7- methylquinoxalin-5-yl)ethyl)amino)- benzoic acid | 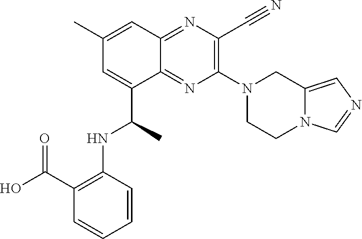 [SMILES: Cc1cc([C@@H](C)Nc2ccccc2C(=O)O)c2nc(N3CCn4cncc4C3)c(C#N)nc2c1] |
| 272 | (R)-2-((1-(2-cyano-3-(6,7-dihydro- pyrazolo[1,5-a]pyrazin-5(4H)-yl)-7- methylquinoxalin-5-yl)ethyl)amino)- benzoic acid | 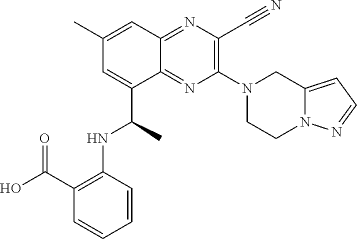 [SMILES: Cc1cc([C@@H](C)Nc2ccccc2C(=O)O)c2nc(N3CCn4nccc4C3)c(C#N)nc2c1] |
| 273 | (R)-2-((1-(2-cyano-7-methyl-3- (piperazin-1-yl)quinoxalin-5-yl)- ethyl)amino)benzoic acid | 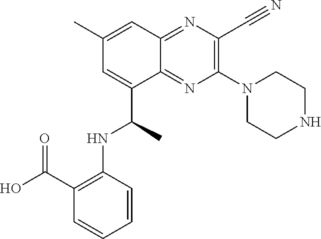 [SMILES: Cc1cc([C@@H](C)Nc2ccccc2C(=O)O)c2nc(N3CCNCC3)c(C#N)nc2c1] |
| 274 | (R)-2-((1-(2-cyano-7-methyl-3-(4- methylpiperazin-1-yl)quinoxalin-5- yl)ethyl)amino)benzoic acid | 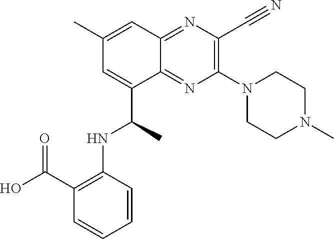 [SMILES: Cc1cc([C@@H](C)Nc2ccccc2C(=O)O)c2nc(N3CCN(C)CC3)c(C#N)nc2c1] |
| 275 | (R)-2-((1-(3-(azepan-1-yl)-2-cyano- 7-methylquinoxalin-5-yl)ethyl)- amino)benzoic acid | 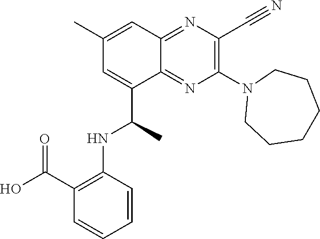 [SMILES: Cc1cc([C@@H](C)Nc2ccccc2C(=O)O)c2nc(N3CCCCCC3)c(C#N)nc2c1] |
| 276 | (R)-2-((1-(2-cyano-7-methyl-3-(2- oxa-8-azaspiro[4.5]decan-8-yl)quin- oxalin-5-yl)ethyl)amino)benzoic acid | 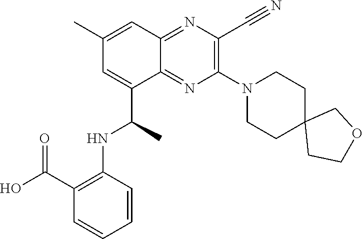 [SMILES: Cc1cc([C@@H](C)Nc2ccccc2C(=O)O)c2nc(N3CCC4(CCOC4)CC3)c(C#N)nc2c1] |
| 277 | (R)-2-((1-(2-cyano-7-methyl-3-(2- oxa-7-azaspiro[3.5]nonan-7-yl)- quinoxalin-5-yl)ethyl)amino)benzoic acid | 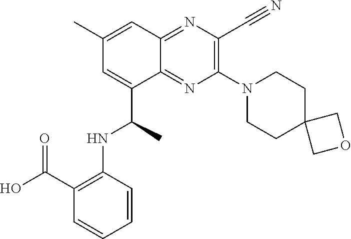 [SMILES: Cc1cc([C@@H](C)Nc2ccccc2C(=O)O)c2nc(N3CCC4(CC3)COC4)c(C#N)nc2c1] |
| 278 | (R)-2-((1-(2-cyano-7-methyl-3-(6- azaspiro[2.5]octan-6-yl)quinoxalin- 5-yl)ethyl)amino)benzoic acid | 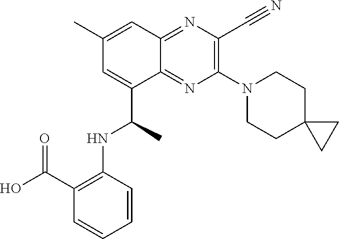 [SMILES: Cc1cc([C@@H](C)Nc2ccccc2C(=O)O)c2nc(N3CCC4(CC3)CC4)c(C#N)nc2c1] |
| 279 | (R)-2-((1-(3-(azetidin-1-yl)-2-cyano- 7-methylquinoxalin-5-yl)ethyl)- amino)benzoic acid | 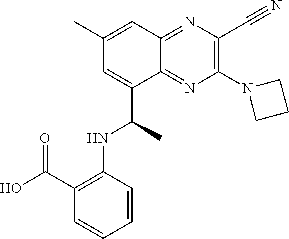 [SMILES: Cc1cc([C@@H](C)Nc2ccccc2C(=O)O)c2nc(N3CCC3)c(C#N)nc2c1] |
| 280 | 2-(((1R)-1-(2-cyano-3-(2-hydroxy- 8-azaspiro[4.5]decan-8-yl)-7-meth- ylquinoxalin-5-yl)ethyl)amino)- benzoic acid | 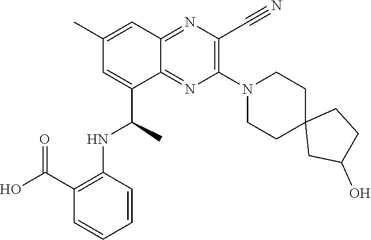 [SMILES: Cc1cc([C@@H](C)Nc2ccccc2C(=O)O)c2nc(N3CCC4(CCC(O)C4)CC3)c(C#N)nc2c1] |
| 281 | (R)-2-((1-(2-cyano-7-methyl-3-(4- methylpiperidin-1-yl)quinoxalin-5- yl)ethyl)amino)benzoic acid | 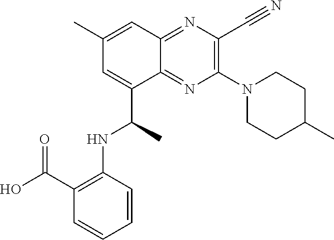 [SMILES: Cc1cc([C@@H](C)Nc2ccccc2C(=O)O)c2nc(N3CCC(C)CC3)c(C#N)nc2c1] |
| 282 | 2-(((1R)-1-(2-cyano-7-methyl-3- (octahydro-2H-4,7-ethanoisoindol- 2-yl)quinoxalin-5-yl)ethyl)amino)- benzoic acid | 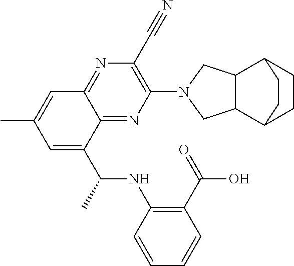 [SMILES: Cc1cc([C@@H](C)Nc2ccccc2C(=O)O)c2nc(N3CC4C5CCC(CC5)C4C3)c(C#N)nc2c1] |
| 283 | 2-(((1R)-1-(2-cyano-3-(6,6-dimethyl- 3-azabicyclo[3.2.0]heptan-3-yl)-7- methylquinoxalin-5-yl)ethyl)amino)- benzoic acid | 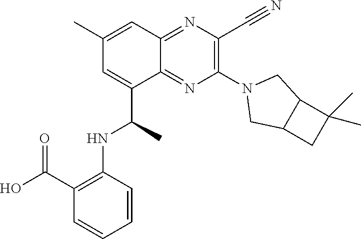 [SMILES: Cc1cc([C@@H](C)Nc2ccccc2C(=O)O)c2nc(N3CC4CC(C)(C)C4C3)c(C#N)nc2c1] |
| 284 | (R)-2-((1-(2-cyano-7-methyl-3-(2- azaspiro[3.3]heptan-2-yl)quinoxalin- 5-yl)ethyl)amino)benzoic acid | 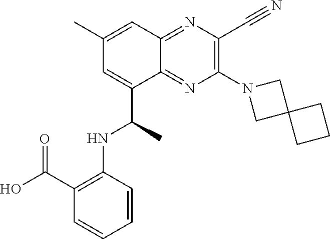 [SMILES: Cc1cc([C@@H](C)Nc2ccccc2C(=O)O)c2nc(N3CC4(CCC4)C3)c(C#N)nc2c1] |
| 285 | (R)-2-((1-(2-cyano-3-(2-azadispiro- [3.1.36.14]decan-2-yl)-7-methylquin- oxalin-5-yl)ethyl)amino)benzoic acid | 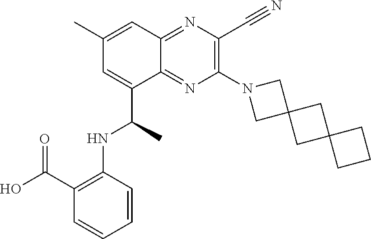 [SMILES: Cc1cc([C@@H](C)Nc2ccccc2C(=O)O)c2nc(N3CC4(C3)CC3(CCC3)C4)c(C#N)nc2c1] |
| 286 | (R)-2-((1-(2-cyano-7-methyl-3-(4- oxa-7-azaspiro[2.5]octan-7-yl)quin- oxalin-5-yl)ethyl)amino)benzoic acid | 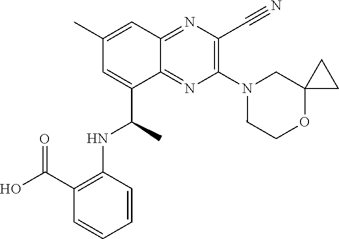 [SMILES: Cc1cc([C@@H](C)Nc2ccccc2C(=O)O)c2nc(N3CCOC4(CC4)C3)c(C#N)nc2c1] |
| 287 | 2-(((1R)-1-(2-cyano-7-methyl-3-(5- methylhexahydropyrrolo[3,4-c]- pyrrol-2(1H)-yl)quinoxalin-5-yl)- ethyl)amino)benzoic acid | 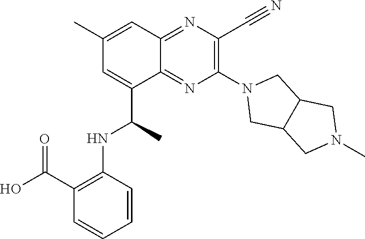 [SMILES: Cc1cc([C@@H](C)Nc2ccccc2C(=O)O)c2nc(N3CC4CN(C)CC4C3)c(C#N)nc2c1] |
| 288 | 2-(((1R)-1-(2-cyano-3-(2-cycloprop- ylmorpholino)-7-methylquinoxalin- 5-yl)ethyl)amino)benzoic acid | 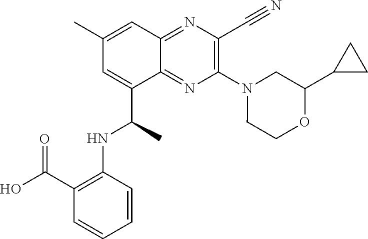 [SMILES: Cc1cc([C@@H](C)Nc2ccccc2C(=O)O)c2nc(N3CCOC(C4CC4)C3)c(C#N)nc2c1] |
| 289 | 2-(((1R)-1-(2-cyano-3-(2-cycloprop- yl-6-methylmorpholino)-7-methyl- quinoxalin-5-yl)ethyl)amino)benzoic acid | 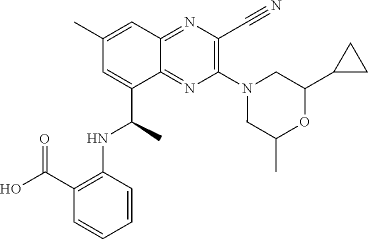 [SMILES: Cc1cc([C@@H](C)Nc2ccccc2C(=O)O)c2nc(N3CC(C)OC(C4CC4)C3)c(C#N)nc2c1] |
| 290 | 2-(((1R)-1-(3-(6-oxa-3-azabicyclo- [3.1.1]heptan-3-yl)-2-cyano-7- methylquinoxalin-5-yl)ethyl)amino)- benzoic acid | 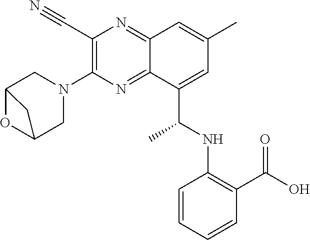 [SMILES: Cc1cc([C@@H](C)Nc2ccccc2C(=O)O)c2nc(N3CC4CC(C3)O4)c(C#N)nc2c1] |
| 291 | (R)-2-((1-(3-(3-azabicyclo[3.1.1]- heptan-3-yl)-2-cyano-7-methylquin- oxalin-5-yl)ethyl)amino)benzoic acid | 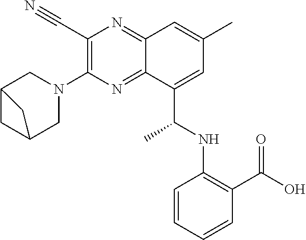 [SMILES: Cc1cc([C@@H](C)Nc2ccccc2C(=O)O)c2nc(N3CC4CC(C4)C3)c(C#N)nc2c1] |
| 292 | 2-(((1R)-1-(3-(3-azabicyclo[3.1.0]- hexan-3-yl)-2-cyano-7-methylquin- oxalin-5-yl)ethyl)amino)benzoic acid | 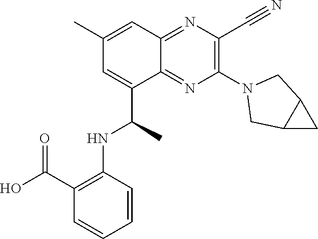 [SMILES: Cc1cc([C@@H](C)Nc2ccccc2C(=O)O)c2nc(N3CC4CC4C3)c(C#N)nc2c1] |
| 293 | (R)-2-((1-(2-cyano-3-(6-(4-cyano- phenyl)-2,6-diazaspiro[3.3]heptan-2- yl)-7-methylquinoxalin-5-yl)ethyl)- amino)benzoic acid | 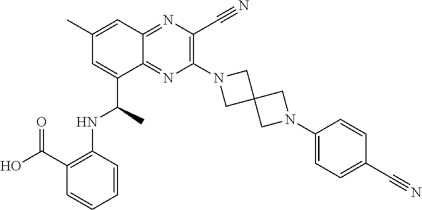 [SMILES: Cc1cc([C@@H](C)Nc2ccccc2C(=O)O)c2nc(N3CC4(CN(c5ccc(C#N)cc5)C4)C3)c(C#N)nc2c1] |
| 294 | 2-(((1R)-1-(2-cyano-7-methyl-3-(2- methylmorpholino)quinoxalin-5-yl)- ethyl)amino)benzoic acid | 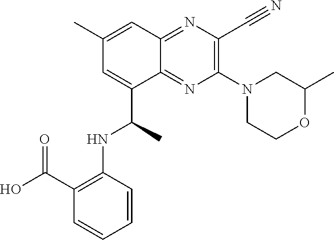 [SMILES: Cc1cc([C@@H](C)Nc2ccccc2C(=O)O)c2nc(N3CCOC(C)C3)c(C#N)nc2c1] |
| 295 | 2-(((1R)-1-(2-cyano-3-(2-cycloprop- yl-2-methylmorpholino)-7-meth- ylquinoxalin-5-yl)ethyl)amino)- benzoic acid | 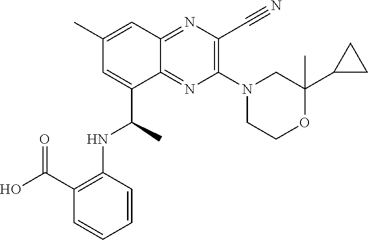 [SMILES: Cc1cc([C@@H](C)Nc2ccccc2C(=O)O)c2nc(N3CCOC(C)(C4CC4)C3)c(C#N)nc2c1] |
| 296 | 2-(((1R)-1-(3-(3,6-diazabicyclo- [3.1.1]heptan-6-yl)-2-cyano-7- methylquinoxalin-5-yl)ethyl)- amino)benzoic acid | 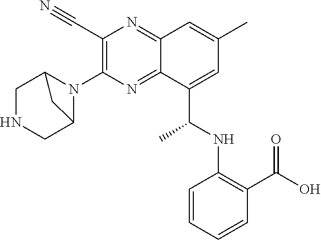 [SMILES: Cc1cc([C@@H](C)Nc2ccccc2C(=O)O)c2nc(N3C4CNCC3C4)c(C#N)nc2c1] |
| 297 | (R)-2-((1-(3-(7-azabicyclo[2.2.1]- heptan-7-yl)-2-cyano-7-methylquin- oxalin-5-yl)ethyl)amino)benzoic acid | 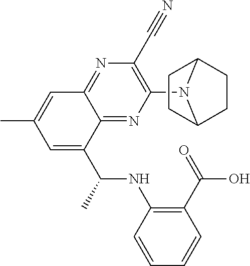 |
| 298 | (R)-2-((1-(3-(2-azaadamantan-2-yl)- 2-cyano-7-methylquinoxalin-5-yl)- ethyl)amino)benzoic acid | 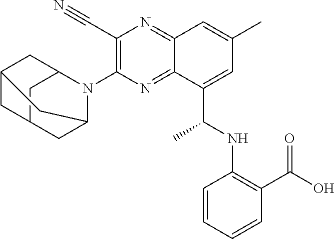 [SMILES: Cc1cc([C@@H](C)Nc2ccccc2C(=O)O)c2nc(N3C4CC5CC(C4)CC3C5)c(C#N)nc2c1] |
| 299 | (R)-2-((1-(2-cyano-3-(1-(methoxy- methyl)-4-methyl-2-azabicyclo- [2.1.1]hexan-2-yl)-7-methylquin- oxalin-5-yl)ethyl)amino)benzoic acid | 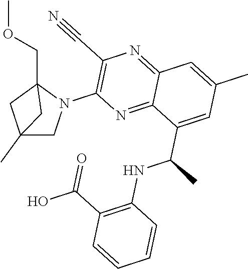 |
| 300 | 2-(((1R)-1-(2-cyano-3-(8-((dimeth- ylamino)methyl)-6-oxa-9-azaspiro- [4.5]decan-9-yl)-7-methylquinoxalin- 5-yl)ethyl)amino)benzoic acid | 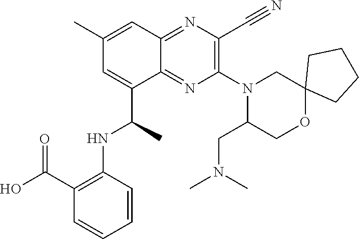 [SMILES: Cc1cc([C@@H](C)Nc2ccccc2C(=O)O)c2nc(N3CC4(CCCC4)OCC3CN(C)C)c(C#N)nc2c1] |
| 301 | (R)-2-((1-(3-(2-azabicyclo[2.2.2]- octan-2-yl)-2-cyano-7-methylquin- oxalin-5-yl)ethyl)amino)benzoic acid | 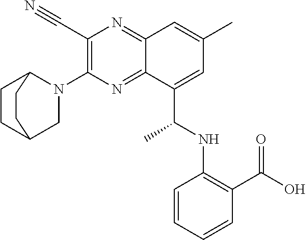 [SMILES: Cc1cc([C@@H](C)Nc2ccccc2C(=O)O)c2nc(N3CC4CCC3CC4)c(C#N)nc2c1] |
| 302 | 2-(((1R)-1-(3-(2-azabicyclo[2.2.1]- heptan-2-yl)-2-cyano-7-methylquin- oxalin-5-yl)ethyl)amino)benzoic acid | 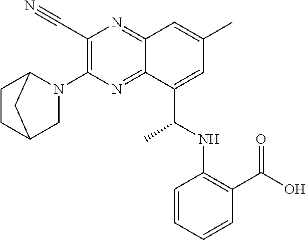 [SMILES: Cc1cc([C@@H](C)Nc2ccccc2C(=O)O)c2nc(N3CC4CCC3C4)c(C#N)nc2c1] |
| 303 | 2-(((1R)-1-(2-cyano-3-(hexahydro- 3,5-methanocyclopenta[b]pyrrol- 1(2H)-yl)-7-methylquinoxalin-5-yl)- ethyl)amino)benzoic acid | 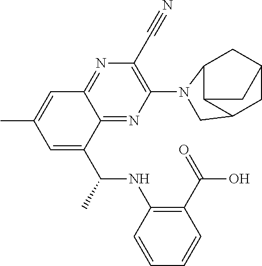 [SMILES: Cc1cc([C@@H](C)Nc2ccccc2C(=O)O)c2nc(N3CC4CC5CC4C3C5)c(C#N)nc2c1] |
| 304 | 2-(((1R)-1-(2-cyano-3-(1-hydroxy- methyl)-6-azabicyclo[3.2.1]octan-6- yl)-7-methylquinoxalin-5-yl)ethyl)- amino)benzoic acid | 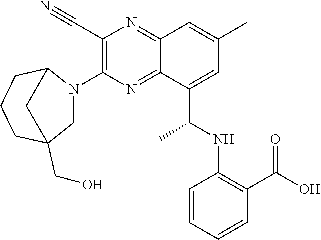 [SMILES: Cc1cc([C@@H](C)Nc2ccccc2C(=O)O)c2nc(N3CC4(CO)CCCC3C4)c(C#N)nc2c1] |
| 305 | (R)-2-((1-(3-(2-azabicyclo[2.1.1]- hexan-2-yl)-2-cyano-7-methylquin- oxalin-5-yl)ethyl)amino)benzoic acid | 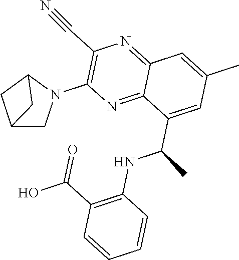 [SMILES: Cc1cc([C@@H](C)Nc2ccccc2C(=O)O)c2nc(N3CC4CC3C4)c(C#N)nc2c1] |
| 306 | 2-(((1R)-1-(2-cyano-7-methyl-3-(3- methylmorpholino)quinoxalin-5-yl)- ethyl)amino)benzoic acid | 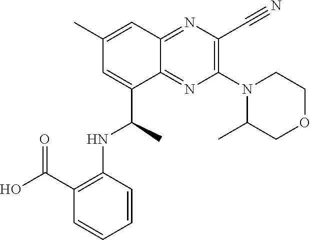 [SMILES: Cc1cc([C@@H](C)Nc2ccccc2C(=O)O)c2nc(N3CCOCC3C)c(C#N)nc2c1] |
| 307 | 2-(((1R)-1-(2-cyano-7-methyl-3-(2- methyl-4-(pyridin-3-ylmethyl)piper- azin-1-yl)quinoxalin-5-yl)ethyl)- amino)benzoic acid | 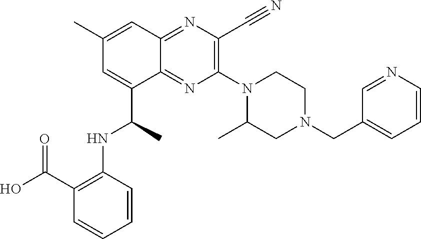 [SMILES: Cc1cc([C@@H](C)Nc2ccccc2C(=O)O)c2nc(N3CCN(Cc4cccnc4)CC3C)c(C#N)nc2c1] |
| 308 | 2-(((1R)-1-(2-cyano-7-methyl-3-(2- methylazetidin-1-yl)quinoxalin-5- yl)ethyl)amino)benzoic acid | 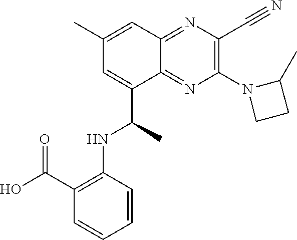 [SMILES: Cc1cc([C@@H](C)Nc2ccccc2C(=O)O)c2nc(N3CCC3C)c(C#N)nc2c1] |
| 309 | 2-(((1R)-1-(2-cyano-3-(2,3-dimethyl- morpholino)-7-methylquinoxalin-5- yl)ethyl)amino)benzoic acid | 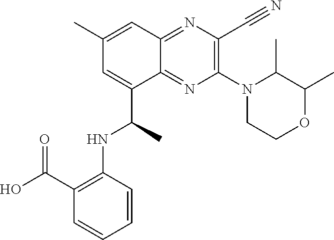 [SMILES: Cc1cc([C@@H](C)Nc2ccccc2C(=O)O)c2nc(N3CCOC(C)C3C)c(C#N)nc2c1] |
| 310 | 2-(((1R)-1-(2-cyano-3-(3-hydroxy-2- (2-methoxyphenyl)pyrrolidin-1-yl)- 7-methylquinoxalin-5-yl)ethyl)- amino)benzoic acid | 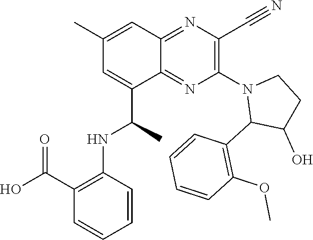 [SMILES: COc1ccccc1C1C(O)CCN1c1nc2c([C@@H](C)Nc3ccccc3C(=O)O)cc(C)cc2nc1C#N] |
| 311 | (R)-2-((1-(2-cyano-3-(3,4-dihydro- quinolin-1(2H)-yl)-7-methylquinox- alin-5-yl)ethyl)amino)benzoic acid | 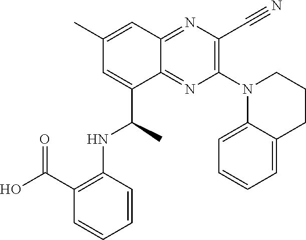 [SMILES: Cc1cc([C@@H](C)Nc2ccccc2C(=O)O)c2nc(N3CCCc4ccccc43)c(C#N)nc2c1] |
| 312 | 2-(((1R)-1-(2-cyano-3-(3-hydroxy- piperidin-1-yl)-7-methylquinoxalin- 5-yl)ethyl)amino)benzoic acid | 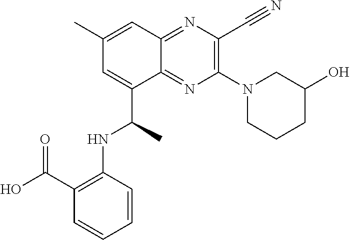 [SMILES: Cc1cc([C@@H](C)Nc2ccccc2C(=O)O)c2nc(N3CCCC(O)C3)c(C#N)nc2c1] |
| 313 | 2-(((1R)-1-(2-cyano-3-(3-hydroxy- 3-methylpiperidin-1-yl)-7-methyl- quinoxalin-5-yl)ethyl)amino)benzoic acid | 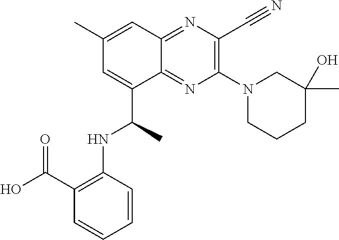 [SMILES: Cc1cc([C@@H](C)Nc2ccccc2C(=O)O)c2nc(N3CCCC(C)(O)C3)c(C#N)nc2c1] |
| 314 | 2-(((1R)-1-(2-cyano-3-(2-(4-hydroxy- 4-methyltetrahydro-2H-pyran-3-yl)- pyrrolidin-1-yl)-7-methylquinoxalin- 5-yl)ethyl)amino)benzoic acid | 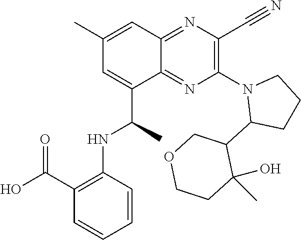 [SMILES: Cc1cc([C@@H](C)Nc2ccccc2C(=O)O)c2nc(N3CCCC3C3COCCC3(C)O)c(C#N)nc2c1] |
| 315 | 2-(((1R)-1-(2-cyano-3-(8-hydroxy-3- azabicyclo[3.2.1]octan-3-yl)-7-meth- quinoxalin-5-yl)ethyl)amino)benzoic acid | 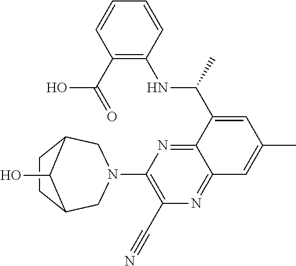 [SMILES: Cc1cc([C@@H](C)Nc2ccccc2C(=O)O)c2nc(N3CC4CCC(C3)C4O)c(C#N)nc2c1] |
| 316 | 2-(((1R)-1-(2-cyano-3-(5-hydroxy- 2-azabicyclo[2.2.2]octan-2-yl)-7- methylquinoxalin-5-yl)ethyl)amino)- benzoic acid | 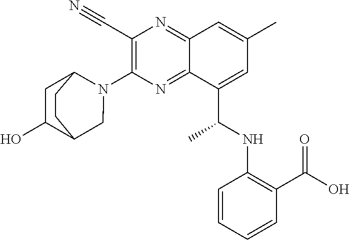 [SMILES: Cc1cc([C@@H](C)Nc2ccccc2C(=O)O)c2nc(N3CC4CCC3CC4O)c(C#N)nc2c1] |
| 317 | 2-(((1R)-1-(2-cyano-3-(6-hydroxy- hexahydro-3,5-methanocyclopenta- [b]pyrrol-1(2H)-yl)-7-methylquin- oxalin-5-yl)ethyl)amino)benzoic acid | 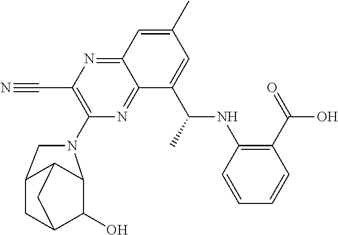 [SMILES: Cc1cc([C@@H](C)Nc2ccccc2C(=O)O)c2nc(N3CC4CC5CC4C3C5O)c(C#N)nc2c1] |
| 318 | 2-(((1R)-1-(2-cyano-3-(6-(4-cyano- phenyl)-3,6-diazabicyclo[3.1.1]- heptan-3-yl)-7-methylquinoxalin-5- yl)ethyl)amino)benzoic acid | 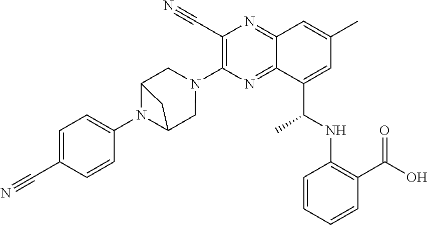 [SMILES: Cc1cc([C@@H](C)Nc2ccccc2C(=O)O)c2nc(N3CC4CC(C3)N4c3ccc(C#N)cc3)c(C#N)nc2c1] |
| 319 | 2-(((1R)-1-(2-cyano-3-(6-hydroxy-6- methyl-3-azabicyclo[3.1.1]heptan-3- yl)-7-methylquinoxalin-5-yl)ethyl)- amino)benzoic acid | 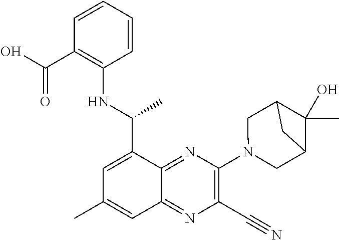 [SMILES: Cc1cc([C@@H](C)Nc2ccccc2C(=O)O)c2nc(N3CC4CC(C3)C4(C)O)c(C#N)nc2c1] |
| 320 | 2-(((1R)-1-(2-cyano-3-(5-(4-cyano- phenyl)-2,5-diazabicyclo[2.2.1]- heptan-2-yl)-7-methylquinoxalin-5- yl)ethyl)amino)benzoic acid | 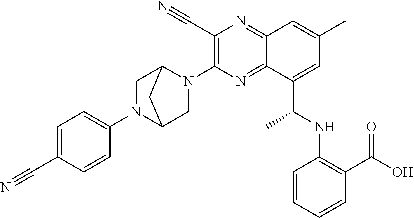 [SMILES: Cc1cc([C@@H](C)Nc2ccccc2C(=O)O)c2nc(N3CC4CC3CN4c3ccc(C#N)cc3)c(C#N)nc2c1] |
| 321 | 2-(((1R)-1-(2-cyano-7-methyl-3-(5- phenyl-2-azabicyclo[2.2.1]heptan-2- yl)quinoxalin-5-yl)ethyl)amino)- benzoic acid | 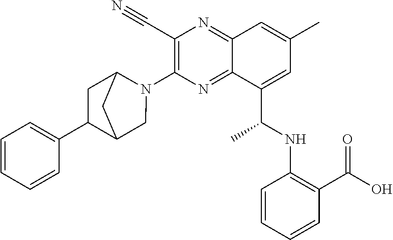 [SMILES: Cc1cc([C@@H](C)Nc2ccccc2C(=O)O)c2nc(N3CC4CC3CC4c3ccccc3)c(C#N)nc2c1] |
| 322 | 2-(((1R)-1-(3-(7-(tert-butyl)-6,7- dihydropyrazolo[1,5-a]pyrimidin- 4(5H)-yl)-2-cyano-7-methylquin- oxalin-5-yl)ethyl)amino)benzoic acid | 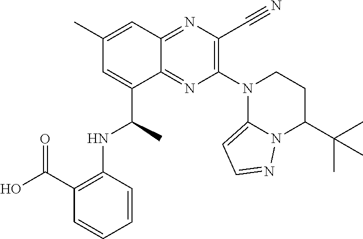 [SMILES: Cc1cc([C@@H](C)Nc2ccccc2C(=O)O)c2nc(N3CCC(C(C)(C)C)n4nccc43)c(C#N)nc2c1] |
| 323 | (R)-2-((1-(2-cyano-7-methyl-3-(1- methyl-1,4,5,7-tetrahydro-6H-pyr- azolo[3,4-c]pyridin-6-yl)quinoxalin- 5-yl)ethyl)amino)benzoic acid | 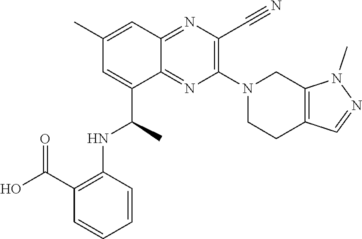 [SMILES: Cc1cc([C@@H](C)Nc2ccccc2C(=O)O)c2nc(N3CCc4cnn(C)c4C3)c(C#N)nc2c1] |
| 324 | (R)-2-((1-(2-cyano-7-methyl-3- (5,6,8,9-tetrahydro-7H-imidazo[1,2- d][1,4]diazepin-7-yl)quinoxalin-5- yl)ethyl)amino)benzoic acid | 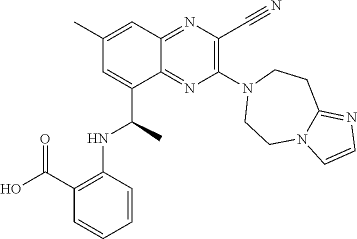 [SMILES: Cc1cc([C@@H](C)Nc2ccccc2C(=O)O)c2nc(N3CCc4nccn4CC3)c(C#N)nc2c1] |
| 325 | (R)-2-((1-(2-cyano-7-methyl-3-(4- (pyridin-2-yl)piperazin-1-yl)quin- oxalin-5-yl)ethyl)amino)benzoic acid | 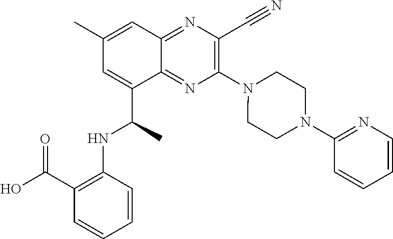 [SMILES: Cc1cc([C@@H](C)Nc2ccccc2C(=O)O)c2nc(N3CCN(c4ccccn4)CC3)c(C#N)nc2c1] |
| 326 | (R)-2-((1-(2-cyano-7-methyl-3-(4- (pyridin-3-yl)piperazin-1-yl)quin- oxalin-5-yl)ethyl)amino)benzoic acid | 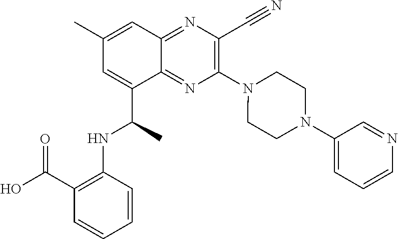 [SMILES: Cc1cc([C@@H](C)Nc2ccccc2C(=O)O)c2nc(N3CCN(c4cccnc4)CC3)c(C#N)nc2c1] |
| 327 | (R)-2-((1-(2-cyano-3-(4-(3-cyano-1- methyl-1H-pyrazol-4-yl)piperazin-1- yl)-7-methylquinoxalin-5-yl)ethyl)- amino)benzoic acid | 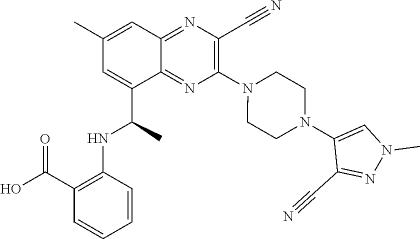 [SMILES: Cc1cc([C@@H](C)Nc2ccccc2C(=O)O)c2nc(N3CCN(c4cn(C)nc4C#N)CC3)c(C#N)nc2c1] |
| 328 | (R)-2-((1-(2-cyano-7-methyl-3-(4- (1-methyl-1H-pyrazol-4-yl)piperazin- 1-yl)quinoxalin-5-yl)ethyl)amino)- benzoic acid | 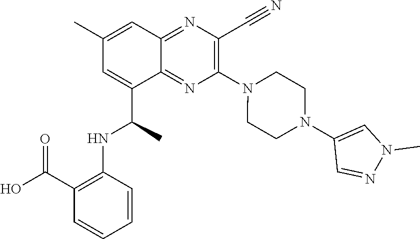 [SMILES: Cc1cc([C@@H](C)Nc2ccccc2C(=O)O)c2nc(N3CCN(c4cnn(C)c4)CC3)c(C#N)nc2c1] |
| 329 | (R)-2-((1-(2-cyano-7-methyl-3-(4- (pyridin-4-yl)piperazin-1-yl)quin- oxalin-5-yl)ethyl)amino)benzoic acid | 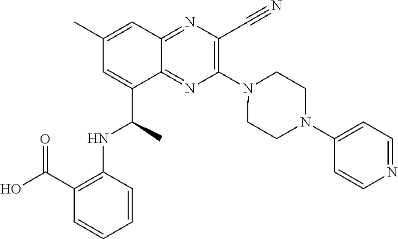 [SMILES: Cc1cc([C@@H](C)Nc2ccccc2C(=O)O)c2nc(N3CCN(c4ccncc4)CC3)c(C#N)nc2c1] |
| 330 | (R)-2-((1-(2-cyano-7-methyl-3-(4- phenylpiperazin-1-yl)quinoxalin-5- yl)ethyl)amino)benzoic acid | 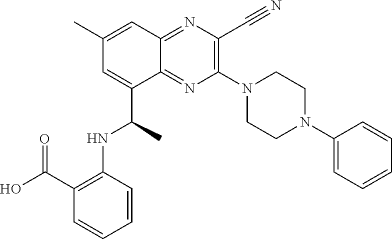 [SMILES: Cc1cc([C@@H](C)Nc2ccccc2C(=O)O)c2nc(N3CCN(c4ccccc4)CC3)c(C#N)nc2c1] |
| 331 | (R)-2-((1-(3-(4-(4-(aminomethyl)- phenyl)piperazin-1-yl)-2-cyano-7- methylquinoxalin-5-yl)ethyl)amino)- benzoic acid | 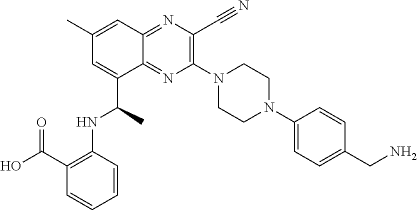 [SMILES: Cc1cc([C@@H](C)Nc2ccccc2C(=O)O)c2nc(N3CCN(c4ccc(CN)cc4)CC3)c(C#N)nc2c1] |
| 332 | (R)-2-((1-(2-cyano-7-methyl-3-(4- (3-methylisoxazol-5-yl)piperazin-1- yl)quinoxalin-5-yl)ethyl)amino)- benzoic acid | 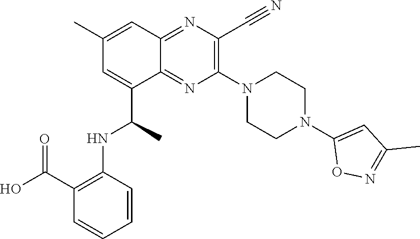 [SMILES: Cc1cc([C@@H](C)Nc2ccccc2C(=O)O)c2nc(N3CCN(c4cc(C)no4)CC3)c(C#N)nc2c1] |
| 333 | (R)-2-((1-(2-cyano-3-(4-(3-cyano- phenyl)piperazin-1-yl)-7-methylquin- oxalin-5-yl)ethyl)amino)benzoic acid | 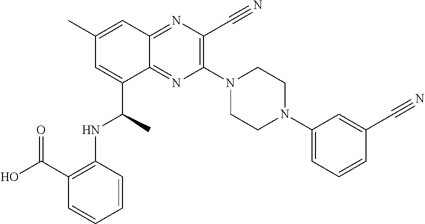 [SMILES: Cc1cc([C@@H](C)Nc2ccccc2C(=O)O)c2nc(N3CCN(c4cccc(C#N)c4)CC3)c(C#N)nc2c1] |
| 334 | (R)-2-((1-(2-cyano-3-(4-(2,6-dimeth- ylphenyl)piperazin-1-yl)-7-methyl- quinoxalin-5-yl)ethyl)amino)benzoic acid | 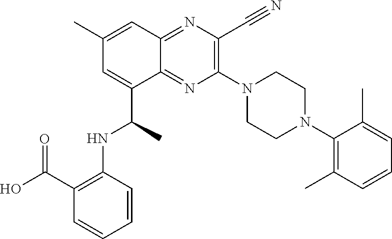 [SMILES: Cc1cc([C@@H](C)Nc2ccccc2C(=O)O)c2nc(N3CCN(c4c(C)cccc4C)CC3)c(C#N)nc2c1] |
| 335 | (R)-2-((1-(2-cyano-7-methyl-3-(4-(o- tolyl)piperazin-1-yl)quinoxalin-5-yl)- ethyl)amino)benzoic acid | 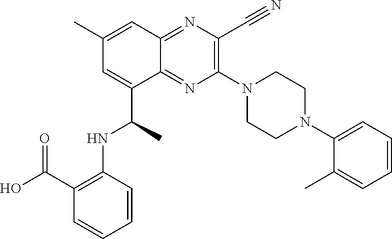 [SMILES: Cc1cc([C@@H](C)Nc2ccccc2C(=O)O)c2nc(N3CCN(c4ccccc4C)CC3)c(C#N)nc2c1] |
| 336 | (R)-2-((1-(2-cyano-3-(4-(3-cyano-2- methylphenyl)piperazin-1-yl)-7- methylquinoxalin-5-yl)ethyl)amino)- benzoic acid | 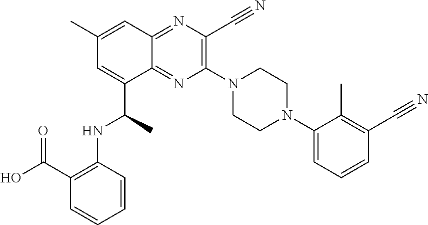 [SMILES: Cc1cc([C@@H](C)Nc2ccccc2C(=O)O)c2nc(N3CCN(c4cccc(C#N)c4C)CC3)c(C#N)nc2c1] |
| 337 | (R)-2-((1-(2-cyano-3-(4-(2-cyanopyr- idin-3-yl)piperazin-1-yl)-7-methyl- quinoxalin-5-yl)ethyl)amino)benzoic acid | 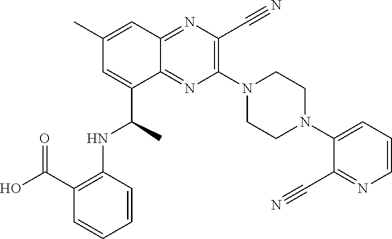 [SMILES: Cc1cc([C@@H](C)Nc2ccccc2C(=O)O)c2nc(N3CCN(c4cccnc4C#N)CC3)c(C#N)nc2c1] |
| 338 | (R)-2-((1-(2-cyano-3-(4-(5-cyano- pyrimidin-2-yl)piperazin-1-yl)-7- methylquinoxalin-5-yl)ethyl)amino)- benzoic acid | 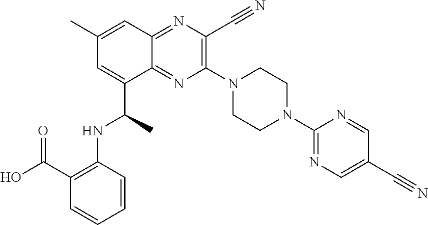 [SMILES: Cc1cc([C@@H](C)Nc2ccccc2C(=O)O)c2nc(N3CCN(c4ncc(C#N)cn4)CC3)c(C#N)nc2c1] |
| 339 | (R)-2-((1-(2-cyano-3-(4-(3-cyano-1- methyl-1H-indazol-7-yl)piperazin-1- yl)-7-methylquinoxalin-5-yl)ethyl)- amino)benzoic acid | 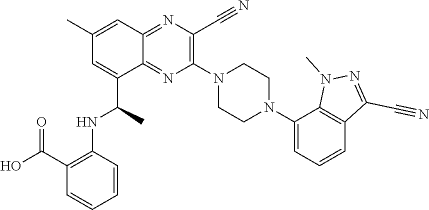 [SMILES: Cc1cc([C@@H](C)Nc2ccccc2C(=O)O)c2nc(N3CCN(c4cccc5c(C#N)nn(C)c45)CC3)c(C#N)nc2c1] |
| 340 | (R)-2-((1-(2-cyano-3-(4-(2-cyano- phenyl)piperazin-1-yl)-7-methyl- quinoxalin-5-yl)ethyl)amino)benzoic acid | 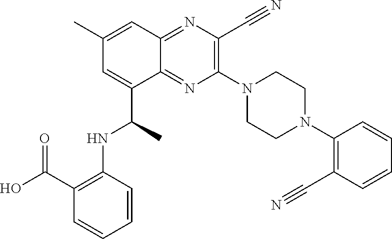 [SMILES: Cc1cc([C@@H](C)Nc2ccccc2C(=O)O)c2nc(N3CCN(c4ccccc4C#N)CC3)c(C#N)nc2c1] |
| 341 | (R)-2-((1-(2-cyano-7-methyl-3-(4-(4- (methylcarbamoyl)phenyl)piperazin- 1-yl)quinoxalin-5-yl)ethyl)amino)- benzoic acid | 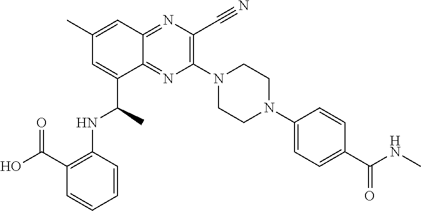 [SMILES: CNC(=O)c1ccc(N2CCN(c3nc4c([C@@H](C)Nc5ccccc5C(=O)O)cc(C)cc4nc3C#N)CC2)cc1] |
| 342 | (R)-2-((1-(3-(4-(4-carbamoylphenyl)- piperazin-1-yl)-2-cyano-7-methyl- quinoxalin-5-yl)ethyl)amino)benzoic acid | 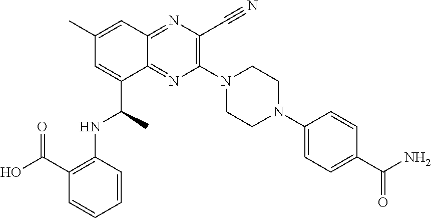 [SMILES: Cc1cc([C@@H](C)Nc2ccccc2C(=O)O)c2nc(N3CCN(c4ccc(C(N)=O)cc4)CC3)c(C#N)nc2c1] |
| 343 | (R)-2-((1-(2-cyano-3-(4-(4-cyano- phenyl)piperazin-1-yl)-7-methylquin- oxalin-5-yl)ethyl)amino)benzoic acid | 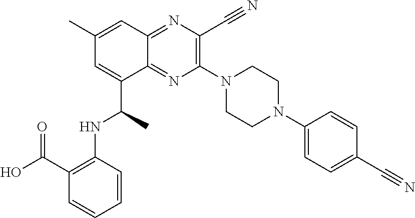 [SMILES: Cc1cc([C@@H](C)Nc2ccccc2C(=O)O)c2nc(N3CCN(c4ccc(C#N)cc4)CC3)c(C#N)nc2c1] |
| 344 | (R)-2-((1-(2-cyano-3-(4-(4-cyano-2- methylphenyl)piperazin-1-yl)-7- methylquinoxalin-5-yl)ethyl)amino)- benzoic acid | 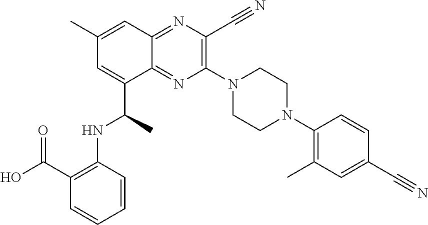 [SMILES: Cc1cc([C@@H](C)Nc2ccccc2C(=O)O)c2nc(N3CCN(c4ccc(C#N)cc4C)CC3)c(C#N)nc2c1] |
| 345 | (R)-2-((1-(2-cyano-3-(4-(8-cyanoiso- quinolin-5-yl)piperazin-1-yl)-7- methylquinoxalin-5-yl)ethyl)amino)- benzoic acid | 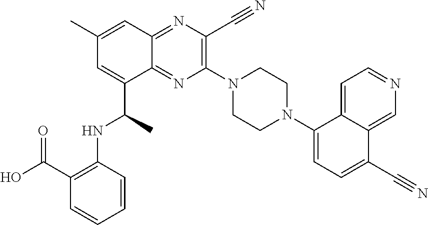 [SMILES: Cc1cc([C@@H](C)Nc2ccccc2C(=O)O)c2nc(N3CCN(c4ccc(C#N)c5cnccc45)CC3)c(C#N)nc2c1] |
| 346 | (R)-2-((1-(2-cyano-3-(4-(4-cyano-7- hydroxynaphthalen-1-yl)piperazin-1- yl)-7-methylquinoxalin-5-yl)ethyl)- amino)benzoic acid | 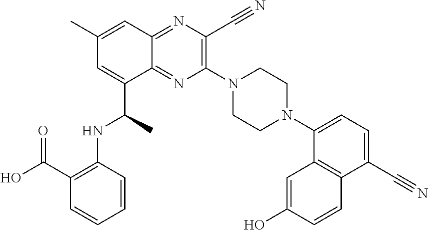 [SMILES: Cc1cc([C@@H](C)Nc2ccccc2C(=O)O)c2nc(N3CCN(c4ccc(C#N)c5ccc(O)cc45)CC3)c(C#N)nc2c1] |
| 347 | (R)-2-((1-(2-cyano-3-(4-(4-cyano-6- hydroxynaphthalen-1-yl)piperazin-1- yl)-7-methylquinoxalin-5-yl)ethyl)- amino)benzoic acid | 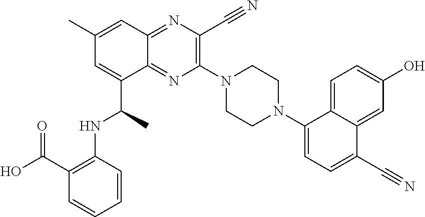 [SMILES: Cc1cc([C@@H](C)Nc2ccccc2C(=O)O)c2nc(N3CCN(c4ccc(C#N)c5cc(O)ccc45)CC3)c(C#N)nc2c1] |
| 348 | (R)-2-((1-(2-cyano-3-(4-(4,6-dicyano- naphthalen-1-yl)piperazin-1-yl)-7- methylquinoxalin-5-yl)ethyl)amino)- benzoic acid | 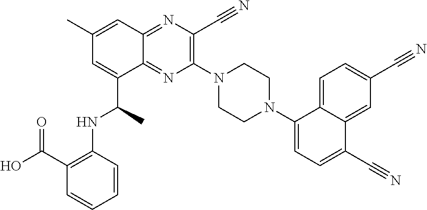 [SMILES: Cc1cc([C@@H](C)Nc2ccccc2C(=O)O)c2nc(N3CCN(c4ccc(C#N)c5cc(C#N)ccc45)CC3)c(C#N)nc2c1] |
| 349 | (R)-2-((1-(2-cyano-7-methyl-3-(4- (oxetan-3-yl)piperazin-1-yl)quin- oxalin-5-yl)ethyl)amino)benzoic acid | 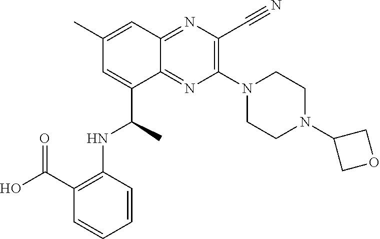 [SMILES: Cc1cc([C@@H](C)Nc2ccccc2C(=O)O)c2nc(N3CCN(C4COC4)CC3)c(C#N)nc2c1] |
| 350 | (R)-2-((1-(2-cyano-7-methyl-3-(4- (tetrahydro-2H-pyran-4-yl)piperazin- 1-yl)quinoxalin-5-yl)ethyl)amino)- benzoic acid | 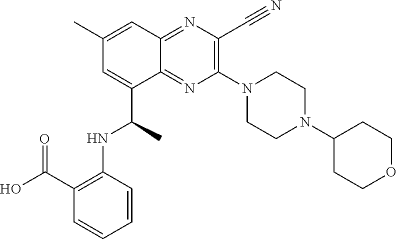 [SMILES: Cc1cc([C@@H](C)Nc2ccccc2C(=O)O)c2nc(N3CCN(C4CCOCC4)CC3)c(C#N)nc2c1] |
| 351 | (R)-2-((1-(2-cyano-3-(4-cycloprop- ylpiperazin-1-yl)-7-methylquinoxalin- 5-yl)ethyl)amino)benzoic acid | 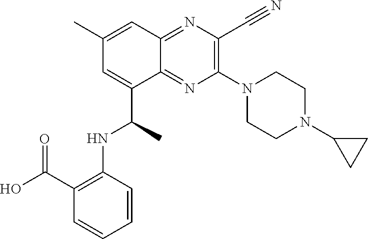 [SMILES: Cc1cc([C@@H](C)Nc2ccccc2C(=O)O)c2nc(N3CCN(C4CC4)CC3)c(C#N)nc2c1] |
| 352 | (R)-2-((1-(2-cyano-7-methyl-3-(4-(1- methyl-1H-pyrazole-4-carbonyl)- piperazin-1-yl)quinoxalin-5-yl)- ethyl)amino)benzoic acid | 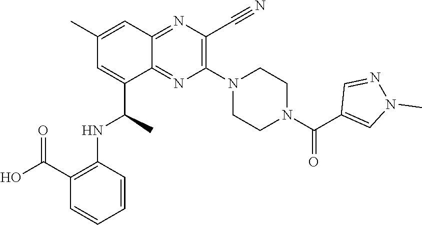 [SMILES: Cc1cc([C@@H](C)Nc2ccccc2C(=O)O)c2nc(N3CCN(C(=O)c4cnn(C)c4)CC3)c(C#N)nc2c1] |
| 353 | (R)-2-((1-(3-(4-benzoylpiperazin-1- yl)-2-cyano-7-methylquinoxalin-5- yl)ethyl)amino)benzoic acid | 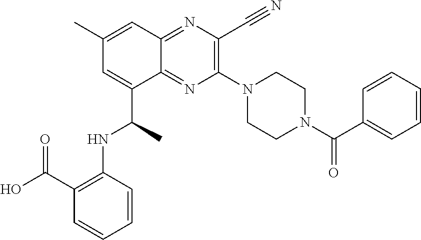 [SMILES: Cc1cc([C@@H](C)Nc2ccccc2C(=O)O)c2nc(N3CCN(C(=O)c4ccccc4)CC3)c(C#N)nc2c1] |
| 354 | (R)-2-((1-(2-cyano-3-(4-(cyclo- pentanecarbonyl)piperazin-1-yl)-7- methylquinoxalin-5-yl)ethyl)amino)- benzoic acid | 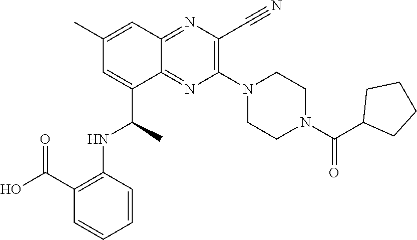 [SMILES: Cc1cc([C@@H](C)Nc2ccccc2C(=O)O)c2nc(N3CCN(C(=O)C4CCCC4)CC3)c(C#N)nc2c1] |
| 355 | (R)-2-((1-(2-cyano-3-(4-(cyclo- propanecarbonyl)piperazin-1-yl)-7- methylquinoxalin-5-yl)ethyl)amino)- benzoic acid | 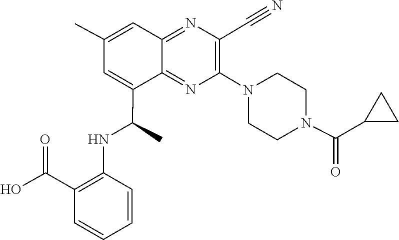 [SMILES: Cc1cc([C@@H](C)Nc2ccccc2C(=O)O)c2nc(N3CCN(C(=O)C4CC4)CC3)c(C#N)nc2c1] |
| 356 | (R)-2-((1-(3-(4-acetylpiperazin-1-yl)- 2-cyano-7-methylquinoxalin-5-yl)- ethyl)amino)benzoic acid | 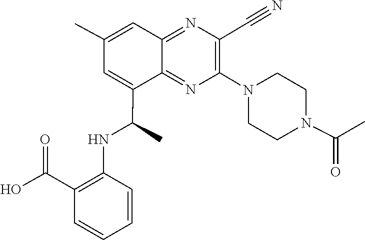 [SMILES: CC(=O)N1CCN(c2nc3c([C@@H](C)Nc4ccccc4C(=O)O)cc(C)cc3nc2C#N)CC1] |
| 357 | (R)-2-((1-(3-(4-(1H-pyrazol-1-yl)- piperidin-1-yl)-2-cyano-7-methyl- quinoxalin-5-yl)ethyl)amino)benzoic acid | 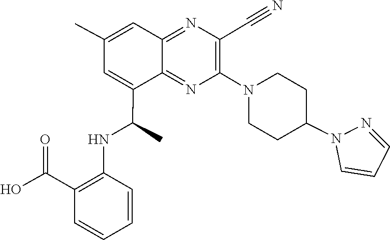 [SMILES: Cc1cc([C@@H](C)Nc2ccccc2C(=O)O)c2nc(N3CCC(n4cccn4)CC3)c(C#N)nc2c1] |
| 358 | (R)-2-((1-(2-cyano-7-methyl-3-(4-(4- methyl-1H-pyrazol-1-yl)piperidin-1- yl)quinoxalin-5-yl)ethyl)amino)- benzoic acid | 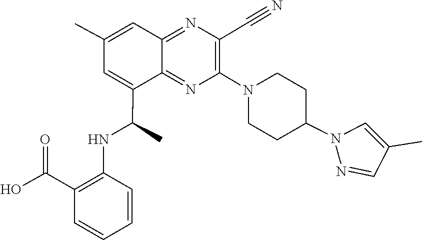 [SMILES: Cc1cc([C@@H](C)Nc2ccccc2C(=O)O)c2nc(N3CCC(n4cc(C)cn4)CC3)c(C#N)nc2c1] |
| 359 | (R)-2-((1-(2-cyano-7-methyl-3-(4-(1- methyl-1H-pyrazol-3-yl)piperidin-1- yl)quinoxalin-5-yl)ethyl)amino)- benzoic acid | 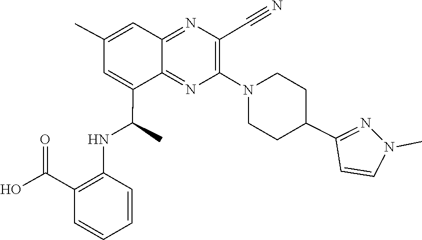 [SMILES: Cc1cc([C@@H](C)Nc2ccccc2C(=O)O)c2nc(N3CCC(c4ccn(C)n4)CC3)c(C#N)nc2c1] |
| 360 | (R)-2-((1-(3-(4-(1H-pyrazol-3-yl)- piperidin-1-yl)-2-cyano-7-methyl- quinoxalin-5-yl)ethyl)amino)benzoic acid | 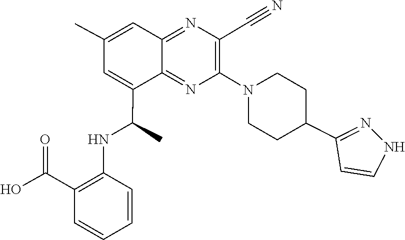 [SMILES: Cc1cc([C@@H](C)Nc2ccccc2C(=O)O)c2nc(N3CCC(c4cc[nH]n4)CC3)c(C#N)nc2c1] |
| 361 | (R)-2-((1-(2-cyano-7-methyl-3-(4-(1- methyl-1H-pyrazol-5-yl)piperidin-1- yl)quinoxalin-5-yl)ethyl)amino)- benzoic acid | 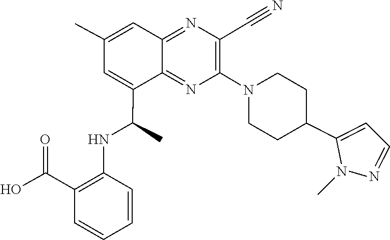 [SMILES: Cc1cc([C@@H](C)Nc2ccccc2C(=O)O)c2nc(N3CCC(c4ccnn4C)CC3)c(C#N)nc2c1] |
| 362 | (R)-2-((1-(2-cyano-3-(4-(4-methoxy- phenyl)piperidin-1-yl)-7-methylquin- oxalin-5-yl)ethyl)amino)benzoic acid | 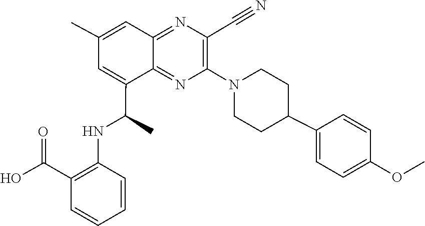 [SMILES: COc1ccc(C2CCN(c3nc4c([C@@H](C)Nc5ccccc5C(=O)O)cc(C)cc4nc3C#N)CC2)cc1] |
| 363 | (R)-2-((1-(2-cyano-3-(4-methoxy- piperidin-1-yl)-7-methylquinoxalin- 5-yl)ethyl)amino)benzoic acid | 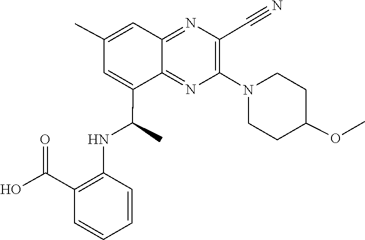 [SMILES: COC1CCN(c2nc3c([C@@H](C)Nc4ccccc4C(=O)O)cc(C)cc3nc2C#N)CC1] |
| 364 | (R)-2-((1-(2-cyano-3-(4-hydroxy- piperidin-1-yl)-7-methylquinoxalin- 5-yl)ethyl)amino)benzoic acid | 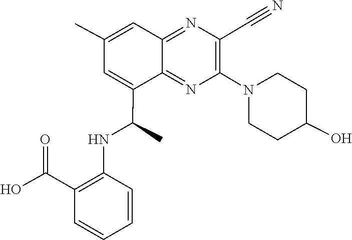 [SMILES: Cc1cc([C@@H](C)Nc2ccccc2C(=O)O)c2nc(N3CCC(O)CC3)c(C#N)nc2c1] |
| 365 | (R)-2-((1-(3-([1,4′-bipiperidin]-1′-yl)- 2-cyano-7-methylquinoxalin-5-yl)- ethyl)amino)benzoic acid | 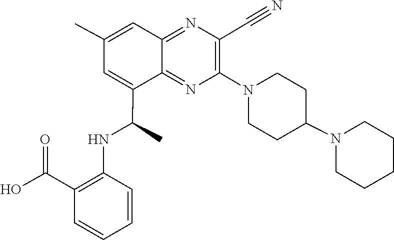 [SMILES: Cc1cc([C@@H](C)Nc2ccccc2C(=O)O)c2nc(N3CCC(N4CCCCC4)CC3)c(C#N)nc2c1] |
| 366 | (R)-2-((1-(2-cyano-3-(4-(dimethyl- amino)piperidin-1-yl)-7-methylquin- oxalin-5-yl)ethyl)amino)benzoic acid | 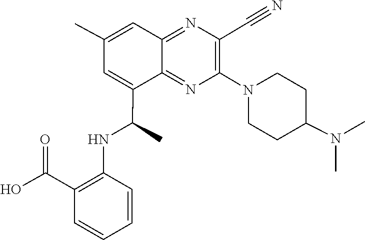 [SMILES: Cc1cc([C@@H](C)Nc2ccccc2C(=O)O)c2nc(N3CCC(N(C)C)CC3)c(C#N)nc2c1] |
| 367 | (R)-2-((1-(2-cyano-3-(4-cyclopentyl- piperidin-1-yl)-7-methylquinoxalin- 5-yl)ethyl)amino)benzoic acid | 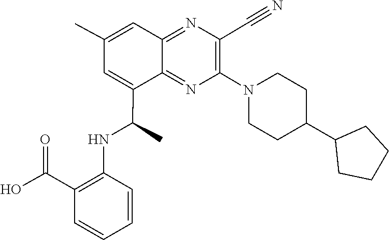 [SMILES: Cc1cc([C@@H](C)Nc2ccccc2C(=O)O)c2nc(N3CCC(C4CCCC4)CC3)c(C#N)nc2c1] |
| 368 | (R)-2-((1-(2-cyano-3-(4-cyclobutyl- piperidin-1-yl)-7-methylquinoxalin- 5-yl)ethyl)amino)benzoic acid | 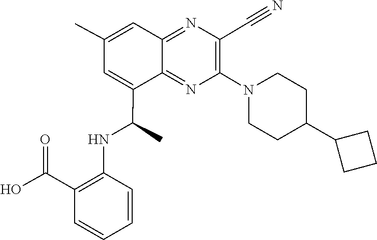 [SMILES: Cc1cc([C@@H](C)Nc2ccccc2C(=O)O)c2nc(N3CCC(C4CCC4)CC3)c(C#N)nc2c1] |
| 369 | (R)-2-((1-(2-cyano-3-(4-cyanopiper- idin-1-yl)-7-methylquinoxalin-5-yl)- ethyl)amino)benzoic acid | 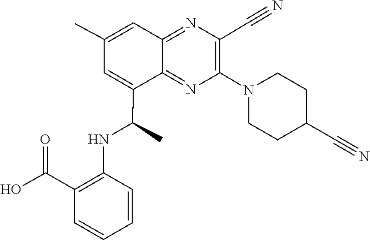 [SMILES: Cc1cc([C@@H](C)Nc2ccccc2C(=O)O)c2nc(N3CCC(C#N)CC3)c(C#N)nc2c1] |
| 370 | (R)-2-((1-(2-cyano-7-methyl-3-(1- oxa-8-azaspiro[4.5]decan-8-yl)quin- oxalin-5-yl)ethyl)amino)benzoic acid | 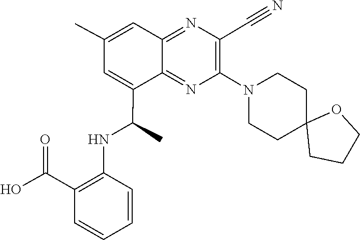 [SMILES: Cc1cc([C@@H](C)Nc2ccccc2C(=O)O)c2nc(N3CCC4(CCCO4)CC3)c(C#N)nc2c1] |
| 371 | (R)-2-((1-(2-cyano-7-methyl-3-(1- oxa-7-azaspiro[3.5]nonan-7-yl)- quinoxalin-5-yl)ethyl)amino)benzoic acid | 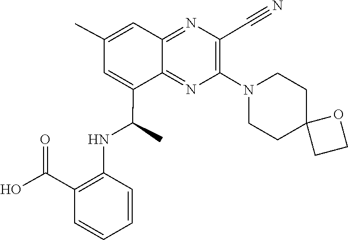 [SMILES: Cc1cc([C@@H](C)Nc2ccccc2C(=O)O)c2nc(N3CCC4(CCO4)CC3)c(C#N)nc2c1] |
| 372 | (R)-2-((1-(2-cyano-3-(4-methoxy-4- methylpiperidin-1-yl)-7-methylquin- oxalin-5-yl)ethyl)amino)benzoic acid | 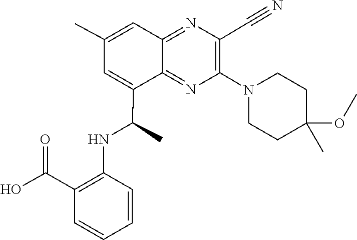 [SMILES: COC1(C)CCN(c2nc3c([C@@H](C)Nc4ccccc4C(=O)O)cc(C)cc3nc2C#N)CC1] |
| 373 | (R)-2-((1-(2-cyano-3-(4-cyano-4- methylpiperidin-1-yl)-7-methylquin- oxalin-5-yl)ethyl)amino)benzoic acid | 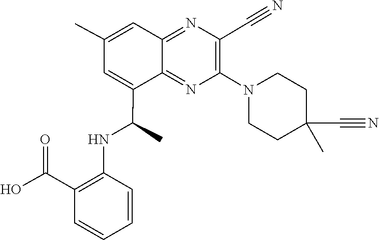 [SMILES: Cc1cc([C@@H](C)Nc2ccccc2C(=O)O)c2nc(N3CCC(C)(C#N)CC3)c(C#N)nc2c1] |
| 374 | 2-(((1R)-1-(2-cyano-7-methyl-3-(3- (pyridin-2-yl)pyrrolidin-1-yl)quin- oxalin-5-yl)ethyl)amino)benzoic acid | 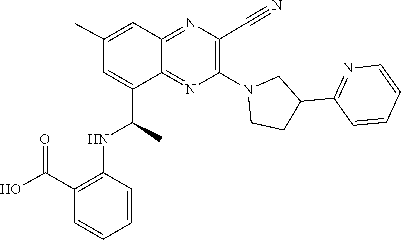 [SMILES: Cc1cc([C@@H](C)Nc2ccccc2C(=O)O)c2nc(N3CCC(c4ccccn4)C3)c(C#N)nc2c1] |
| 375 | 2-(((1R)-1-(2-cyano-7-methyl-3-(3- (pyridin-4-yl)pyrrolidin-1-yl)quin- oxalin-5-yl)ethyl)amino)benzoic acid | 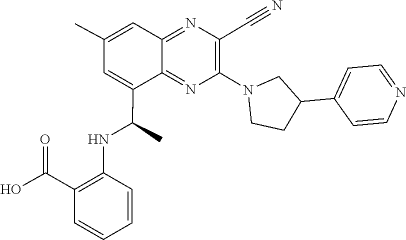 [SMILES: Cc1cc([C@@H](C)Nc2ccccc2C(=O)O)c2nc(N3CCC(c4ccncc4)C3)c(C#N)nc2c1] |
| 376 | 2-(((1R)-1-(2-cyano-7-methyl-3-(3- phenylpyrrolidin-1-yl)quinoxalin-5- yl)ethyl)amino)benzoic acid | 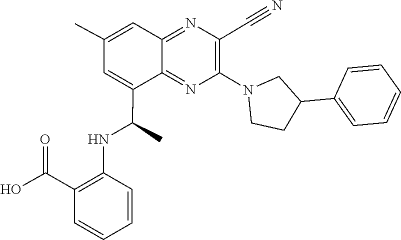 [SMILES: Cc1cc([C@@H](C)Nc2ccccc2C(=O)O)c2nc(N3CCC(c4ccccc4)C3)c(C#N)nc2c1] |
| 377 | 2-(((1R)-1-(2-cyano-3-(3-(1,3- dihydroxypropan-2-yl)pyrrolidin-1- yl)-7-methylquinoxalin-5-yl)ethyl)- amino)benzoic acid | 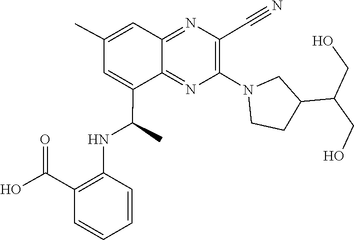 [SMILES: Cc1cc([C@@H](C)Nc2ccccc2C(=O)O)c2nc(N3CCC(C(CO)CO)C3)c(C#N)nc2c1] |
| 378 | 2-(((1R)-1-(2-cyano-7-methyl-3-(1- oxa-6-azaspiro[3.4]octan-6-yl)- quinoxalin-5-yl)ethyl)amino)benzoic acid | 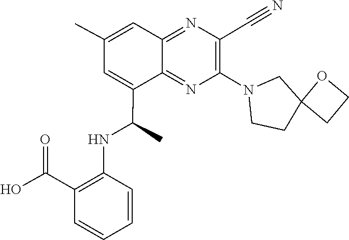 [SMILES: Cc1cc([C@@H](C)Nc2ccccc2C(=O)O)c2nc(N3CCC4(CCO4)C3)c(C#N)nc2c1] |
| 379 | 2-(((1R)-1-(2-cyano-7-methyl-3-(2- oxa-7-azaspiro[4.4]nonan-7-yl)- quinoxalin-5-yl)ethyl)amino)benzoic acid | 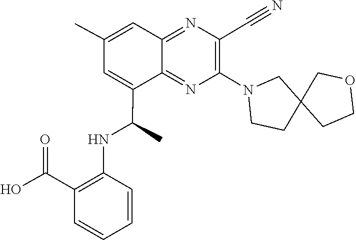 [SMILES: Cc1cc([C@@H](C)Nc2ccccc2C(=O)O)c2nc(N3CCC4(CCOC4)C3)c(C#N)nc2c1] |
| 380 | 2-(((1R)-1-(2-cyano-7-methyl-3- (spiro[bicyclo[2.2.1]heptane-2,3′- pyrrolidin]-1′-yl)quinoxalin-5-yl)- ethyl)amino)benzoic acid | 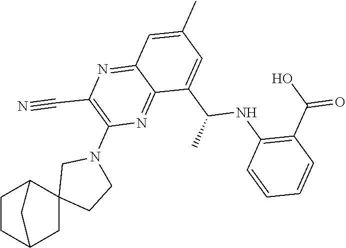 [SMILES: Cc1cc([C@@H](C)Nc2ccccc2C(=O)O)c2nc(N3CCC4(CC5CCC4C5)C3)c(C#N)nc2c1] |
| 381 | 2-(((1R)-1-(2-cyano-3-(8,9- dihydroxy-6-oxa-2-azaspiro[4.5]- decan-2-yl)-7-methylquinoxalin-5- yl)ethyl)amino)benzoic acid | 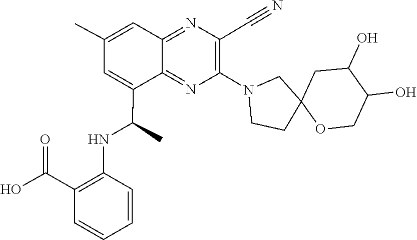 [SMILES: Cc1cc([C@@H](C)Nc2ccccc2C(=O)O)c2nc(N3CCC4(CC(O)C(O)CO4)C3)c(C#N)nc2c1] |
| 382 | 2-(((1R)-1-(2-cyano-3-(4-(4-cyano- phenyl)-3-methylpiperazin-1-yl)-7- methylquinoxalin-5-yl)ethyl)amino)- benzoic acid | 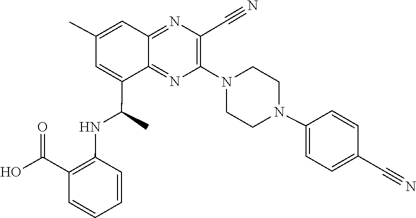 [SMILES: Cc1cc([C@@H](C)Nc2ccccc2C(=O)O)c2nc(N3CCN(c4ccc(C#N)cc4)CC3)c(C#N)nc2c1] |
| 383 | 2-(((1R)-1-(2-cyano-7-methyl-3-(2- methyl-4-phenylpiperazin-1-yl)quin- oxalin-5-yl)ethyl)amino)benzoic acid | 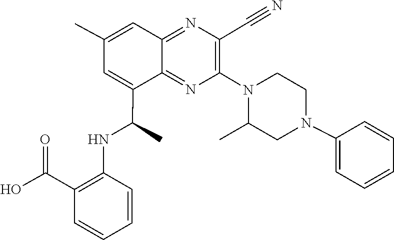 [SMILES: Cc1cc([C@@H](C)Nc2ccccc2C(=O)O)c2nc(N3CCN(c4ccccc4)CC3C)c(C#N)nc2c1] |
| 384 | 2-(((1R)-1-(2-cyano-3-(4-(3-cyano- phenyl)-2-methylpiperazin-1-yl)-7- methylquinoxalin-5-yl)ethyl)amino)- benzoic acid | 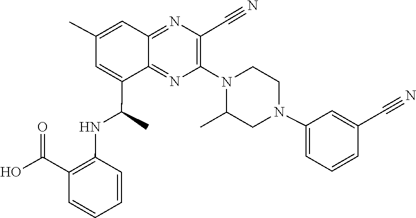 [SMILES: Cc1cc([C@@H](C)Nc2ccccc2C(=O)O)c2nc(N3CCN(c4cccc(C#N)c4)CC3C)c(C#N)nc2c1] |
| 385 | 2-(((1R)-1-(2-cyano-3-(4-(2-cyano- phenyl)-2-methylpiperazin-1-yl)-7- methylquinoxalin-5-yl)ethyl)amino)- benzoic acid | 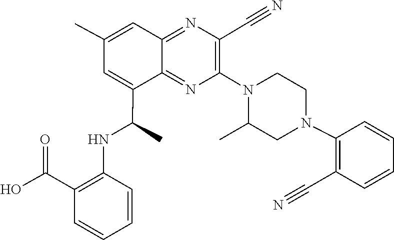 [SMILES: Cc1cc([C@@H](C)Nc2ccccc2C(=O)O)c2nc(N3CCN(c4ccccc4C#N)CC3C)c(C#N)nc2c1] |
| 386 | 2-(((1R)-1-(2-cyano-3-(4-(4-cyano- phenyl)-2-methylpiperazin-1-yl)-7- methylquinoxalin-5-yl)ethyl)amino)- benzoic acid | 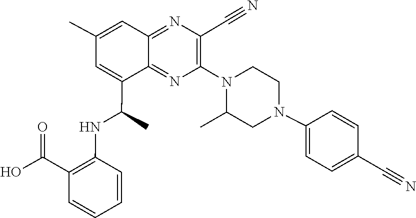 [SMILES: Cc1cc([C@@H](C)Nc2ccccc2C(=O)O)c2nc(N3CCN(c4ccc(C#N)cc4)CC3C)c(C#N)nc2c1] |
| 387 | 2-(((1R)-1-(2-cyano-3-(4-(2-cyano- 5-methylphenyl)-2-methylpiperazin- 1-yl)-7-methylquinoxalin-5-yl)ethyl)- amino)benzoic acid | 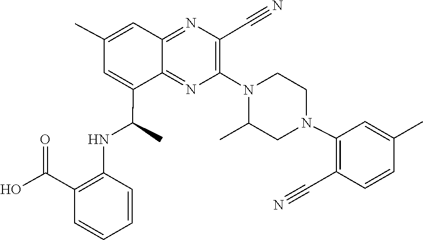 [SMILES: Cc1ccc(C#N)c(N2CCN(c3nc4c([C@@H](C)Nc5ccccc5C(=O)O)cc(C)cc4nc3C#N)C(C)C2)c1] |
| 388 | 2-(((1R)-1-(2-cyano-3-(6-hydroxy-2- azabicyclo[2.2.2]octan-2-yl)-7-meth- ylquinoxalin-5-yl)ethyl)amino)benzoic acid | 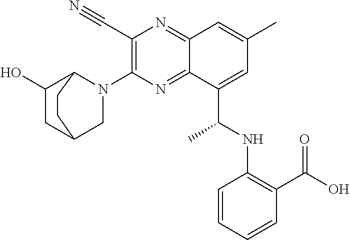 [SMILES: Cc1cc([C@@H](C)Nc2ccccc2C(=O)O)c2nc(N3CC4CCC3C(O)C4)c(C#N)nc2c1] |
| 389 | 2-(((1R)-1-(2-cyano-7-methyl-3- (1,2,3,4-tetrahydro-1,4-epimino- naphthalen-9-yl)quinoxalin-5-yl)- ethyl)amino)benzoic acid | 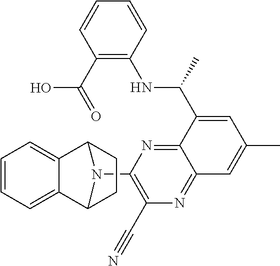 [SMILES: Cc1cc([C@@H](C)Nc2ccccc2C(=O)O)c2nc(N3C4CCC3c3ccccc34)c(C#N)nc2c1] |
| 390 | (R)-2-((1-(2-cyano-3-(5,7-dihydro- 6H-pyrrolo[3,4-b]pyrazin-6-yl)-7- methylquinoxalin-5-yl)ethyl)amino)- benzoic acid | 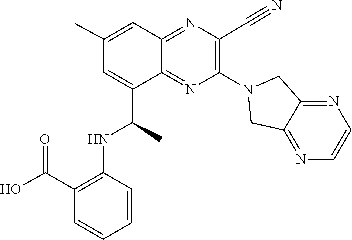 [SMILES: Cc1cc([C@@H](C)Nc2ccccc2C(=O)O)c2nc(N3Cc4nccnc4C3)c(C#N)nc2c1] |
| 391 | (R)-2-((1-(2-cyano-3-(1,3-dihydro- 2H-pyrrolo[3,4-c]pyridin-2-yl)-7- methylquinoxalin-5-yl)ethyl)amino)- benzoic acid | 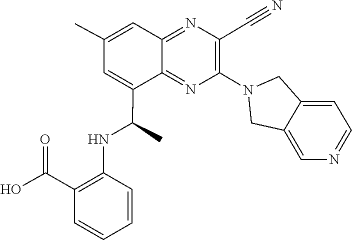 [SMILES: Cc1cc([C@@H](C)Nc2ccccc2C(=O)O)c2nc(N3Cc4ccncc4C3)c(C#N)nc2c1] |
| 392 | (R)-2-((1-(2-cyano-3-(5,7-dihydro- 6H-pyrrolo[3,4-b]pyridin-6-yl)-7- methylquinoxalin-5-yl)ethyl)amino)- benzoic acid | 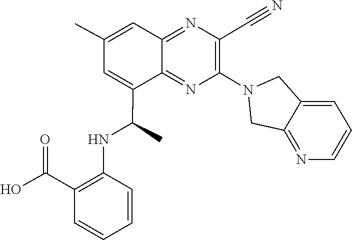 [SMILES: Cc1cc([C@@H](C)Nc2ccccc2C(=O)O)c2nc(N3Cc4cccnc4C3)c(C#N)nc2c1] |
| 393 | (R)-2-((1-(2-cyano-3-(4-cyanoiso- indolin-2-yl)-7-methylquinoxalin-5- yl)ethyl)amino)benzoic acid | 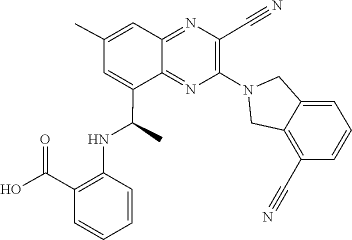 [SMILES: Cc1cc([C@@H](C)Nc2ccccc2C(=O)O)c2nc(N3Cc4cccc(C#N)c4C3)c(C#N)nc2c1] |
| 394 | (R)-2-((1-(2-cyano-3-(5-(hydroxy- methyl)isoindolin-2-yl)-7-methyl- quinoxalin-5-yl)ethyl)amino)benzoic acid | 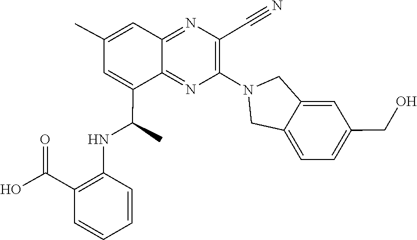 [SMILES: Cc1cc([C@@H](C)Nc2ccccc2C(=O)O)c2nc(N3Cc4ccc(CO)cc4C3)c(C#N)nc2c1] |
| 395 | (R)-2-((1-(2-cyano-7-methyl-3-(5- methylisoindolin-2-yl)quinoxalin-5- yl)ethyl)amino)benzoic acid | 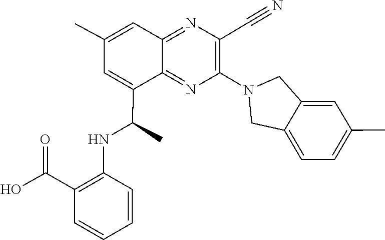 [SMILES: Cc1ccc2c(c1)CN(c1nc3c([C@@H](C)Nc4ccccc4C(=O)O)cc(C)cc3nc1C#N)C2] |
| 396 | (R)-2-((1-(2-cyano-3-(5,6-dimethoxy- isoindolin-2-yl)-7-methylquinoxalin- 5-yl)ethyl)amino)benzoic acid | 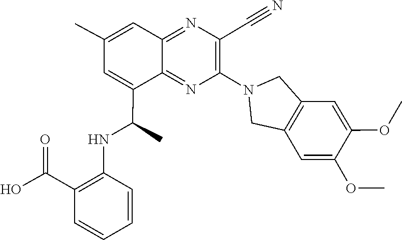 [SMILES: COc1cc2c(cc1OC)CN(c1nc3c([C@@H](C)Nc4ccccc4C(=O)O)cc(C)cc3nc1C#N)C2] |
| 397 | 2-(((1R)-1-(2-cyano-7-methyl-3- (tetrahydro-1H,3H-spiro[pyrrolo[2,1- c][1,4]oxazine-4,5′-[1,3]oxazinan]-3′- yl)quinoxalin-5-yl)ethyl)amino)- benzoic acid | 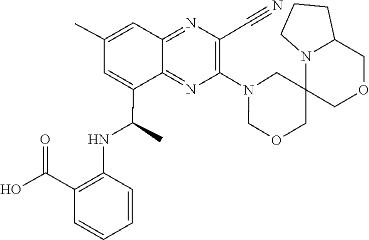 [SMILES: Cc1cc([C@@H](C)Nc2ccccc2C(=O)O)c2nc(N3COCC4(COCC5CCCN54)C3)c(C#N)nc2c1] |
| 398 | 2-(((1R)-1-(2-cyano-3-(decahydro- 2H-cycloocta[c]pyrrol-2-yl)-7- methylquinoxalin-5-yl)ethyl)amino)- benzoic acid | 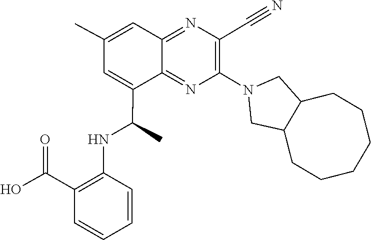 [SMILES: Cc1cc([C@@H](C)Nc2ccccc2C(=O)O)c2nc(N3CC4CCCCCCC4C3)c(C#N)nc2c1] |
| 399 | 2-(((1R)-1-(2-cyano-7-methyl-3-(8- azatricyclo[4.3.0.02,5]nonan-8-yl)- quinoxalin-5-yl)ethyl)amino)benzoic acid | 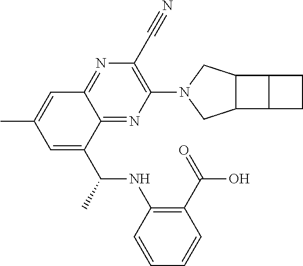 [SMILES: Cc1cc([C@@H](C)Nc2ccccc2C(=O)O)c2nc(N3CC4C5CCC5C4C3)c(C#N)nc2c1] |
| 400 | (R)-2-((1-(3-(1H,3H,4H-3a,6a- (methanooxymethano)furo[3,4-c]- pyrrol-5(6H)-yl)-2-cyano-7-methyl- quinoxalin-5-yl)ethyl)amino)benzoic acid | 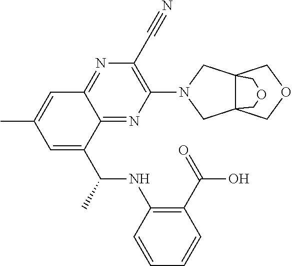 [SMILES: Cc1cc([C@@H](C)Nc2ccccc2C(=O)O)c2nc(N3CC45COCC4(COC5)C3)c(C#N)nc2c1] |
| 401 | 2-(((1R)-1-(2-cyano-3-(8-hydroxy- 6-azaspiro[3.4]octan-6-yl)-7-meth- ylquinoxalin-5-yl)ethyl)amino)- benzoic acid | 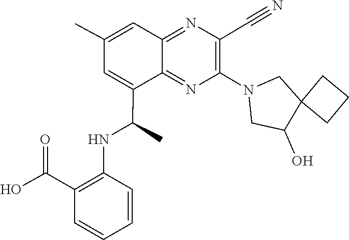 [SMILES: Cc1cc([C@@H](C)Nc2ccccc2C(=O)O)c2nc(N3CC(O)C4(CCC4)C3)c(C#N)nc2c1] |
| 402 | (R)-2-((1-(2-cyano-7-methyl-3-(3- (pyridin-2-yl)azetidin-1-yl)quinoxalin- 5-yl)ethyl)amino)benzoic acid | 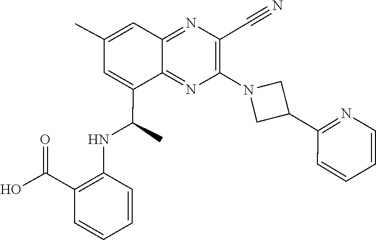 [SMILES: Cc1cc([C@@H](C)Nc2ccccc2C(=O)O)c2nc(N3CC(c4ccccn4)C3)c(C#N)nc2c1] |
| 403 | (R)-2-((1-(2-cyano-7-methyl-3-(3- (pyridin-3-yl)azetidin-1-yl)quinoxalin- 5-yl)ethyl)amino)benzoic acid | 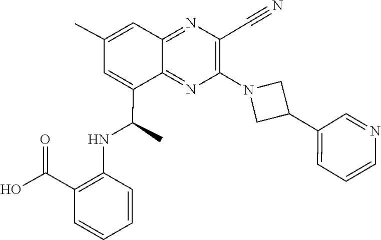 [SMILES: Cc1cc([C@@H](C)Nc2ccccc2C(=O)O)c2nc(N3CC(c4cccnc4)C3)c(C#N)nc2c1] |
| 404 | (R)-2-((1-(2-cyano-7-methyl-3-(3- phenylazetidin-1-yl)quinoxalin-5-yl)- ethyl)amino)benzoic acid | 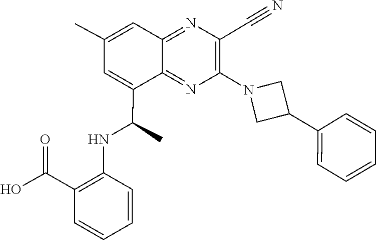 [SMILES: Cc1cc([C@@H](C)Nc2ccccc2C(=O)O)c2nc(N3CC(c4ccccc4)C3)c(C#N)nc2c1] |
| 405 | (R)-2-((1-(2-cyano-3-(3-methoxy- azetidin-1-yl)-7-methylquinoxalin-5- yl)ethyl)amino)benzoic acid | 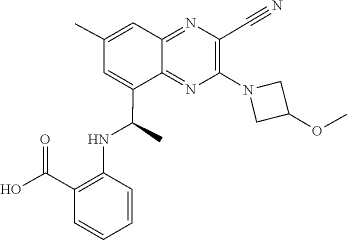 [SMILES: COC1CN(c2nc3c([C@@H](C)Nc4ccccc4C(=O)O)cc(C)cc3nc2C#N)C1] |
| 406 | (R)-2-((1-(2-cyano-3-(3-cyanoazeti- din-1-yl)-7-methylquinoxalin-5-yl)- ethyl)amino)benzoic acid | 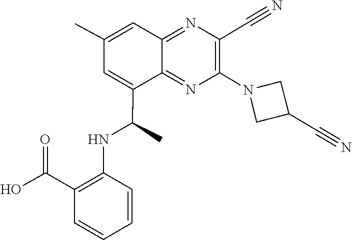 [SMILES: Cc1cc([C@@H](C)Nc2ccccc2C(=O)O)c2nc(N3CC(C#N)C3)c(C#N)nc2c1] |
| 407 | (R)-2-((1-(2-cyano-3-(7-(4-cyano- phenyl)-2,7-diazaspiro[3.5]nonan-2- yl)-7-methylquinoxalin-5-yl)ethyl)- amino)benzoic acid | 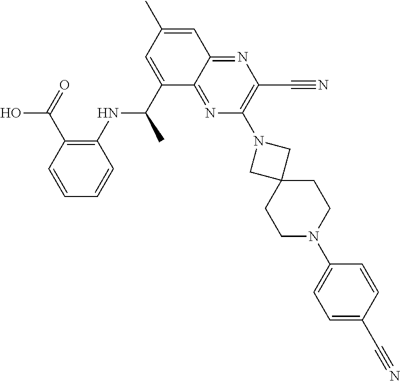 [SMILES: Cc1cc([C@@H](C)Nc2ccccc2C(=O)O)c2nc(N3CC4(CCN(c5ccc(C#N)cc5)CC4)C3)c(C#N)nc2c1] |
| 408 | 2-(((1R)-1-(2-cyano-3-(8-hydroxy- octahydro-2H-4,7-methanoisoindol- 2-yl)-7-methylquinoxalin-5-yl)ethyl)- amino)benzoic acid | 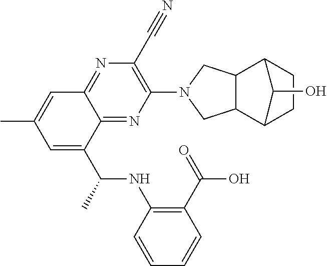 [SMILES: Cc1cc([C@@H](C)Nc2ccccc2C(=O)O)c2nc(N3CC4C5CCC(C5O)C4C3)c(C#N)nc2c1] |
| 409 | 2-(((1R)-1-(2-cyano-7-methyl-3-(5- methyl-2-azabicyclo[2.2.1]heptan-2- yl)quinoxalin-5-yl)ethyl)amino)- benzoic acid | 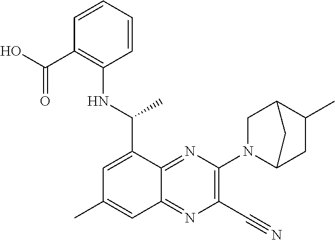 [SMILES: Cc1cc([C@@H](C)Nc2ccccc2C(=O)O)c2nc(N3CC4CC3CC4C)c(C#N)nc2c1] |
| 410 | 2-(((1R)-1-(2-cyano-7-methyl-3-(1′- methyltetrahydrospiro[furo[3,4-b]- pyrrole-3,4′-piperidin]-1(2H)-yl)quin- oxalin-5-yl)ethyl)amino)benzoic acid | 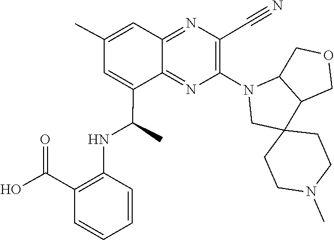 [SMILES: Cc1cc([C@@H](C)Nc2ccccc2C(=O)O)c2nc(N3CC4(CCN(C)CC4)C4COCC43)c(C#N)nc2c1] |
| 411 | 2-(((1R)-1-(2-cyano-3-(1-(hydroxy- methyl)isoindolin-2-yl)-7-methylquin- oxalin-5-yl)ethyl)amino)benzoic acid | 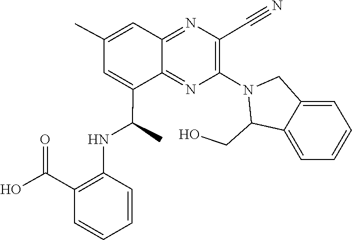 [SMILES: Cc1cc([C@@H](C)Nc2ccccc2C(=O)O)c2nc(N3Cc4ccccc4C3CO)c(C#N)nc2c1] |
| 412 | 2-(((1R)-1-(2-cyano-3-(3-hydroxy- octahydro-1H-indol-1-yl)-7-methyl- quinoxalin-5-yl)ethyl)amino)benzoic acid | 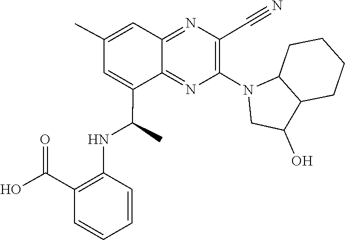 [SMILES: Cc1cc([C@@H](C)Nc2ccccc2C(=O)O)c2nc(N3CC(O)C4CCCCC43)c(C#N)nc2c1] |
| 413 | 2-(((1R)-1-(2-cyano-7-methyl-3-(1- methyloctahydro-2H-isoindol-2-yl)- quinoxalin-5-yl)ethyl)amino)benzoic acid | 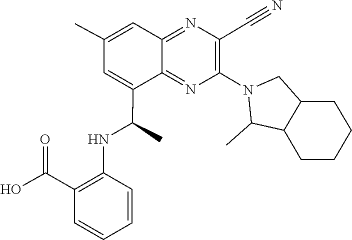 [SMILES: Cc1cc([C@@H](C)Nc2ccccc2C(=O)O)c2nc(N3CC4CCCCC4C3C)c(C#N)nc2c1] |
| 414 | (R)-2-((1-(2-cyano-7-methyl-3-(meth- yl((5,6,8,9-tetrahydro-[1,2,4]triazolo- [4,3-d][1,4]oxazepin-3-yl)methyl)- amino)quinoxalin-5-yl)ethyl)amino)- benzoic acid | 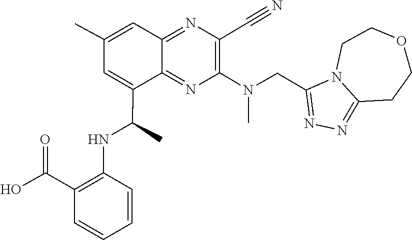 [SMILES: Cc1cc([C@@H](C)Nc2ccccc2C(=O)O)c2nc(N(C)Cc3nnc4n3CCOCC4)c(C#N)nc2c1] |
| 415 | 2-(((1R)-1-(2-cyano-7-methyl-3- (methyl((5,6,7,8-tetrahydroimidazo- [1,2-a]pyridin-7-yl)methyl)amino)- quinoxalin-5-yl)ethyl)amino)benzoic acid | 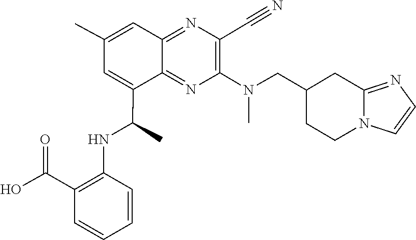 [SMILES: Cc1cc([C@@H](C)Nc2ccccc2C(=O)O)c2nc(N(C)CC3CCn4ccnc4C3)c(C#N)nc2c1] |
| 416 | (R)-2-((1-(2-cyano-3-((cyclopropyl- methyl)(methyl)amino)-7-methyl- quinoxalin-5-yl)ethyl)amino)benzoic acid | 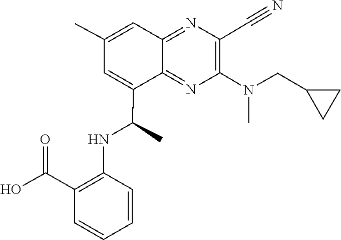 [SMILES: Cc1cc([C@@H](C)Nc2ccccc2C(=O)O)c2nc(N(C)CC3CC3)c(C#N)nc2c1] |
| 417 | 2-(((1R)-1-(2-cyano-3-(((1-hydroxy- 2,3-dihydro-1H-inden-1-yl)methyl)- (methyl)amino)-7-methylquinoxalin- 5-yl)ethyl)amino)benzoic acid | 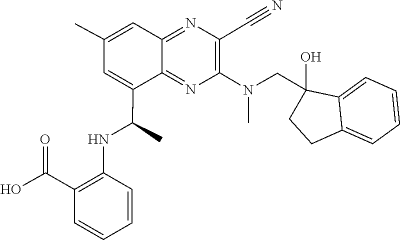 [SMILES: Cc1cc([C@@H](C)Nc2ccccc2C(=O)O)c2nc(N(C)CC3(O)CCc4ccccc43)c(C#N)nc2c1] |
| 418 | (R)-2-((1-(2-cyano-3-(dimethyl- amino)-7-methylquinoxalin-5- yl)ethyl)amino)benzoic acid | 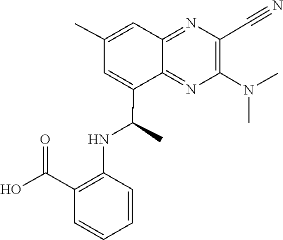 [SMILES: Cc1cc([C@@H](C)Nc2ccccc2C(=O)O)c2nc(N(C)C)c(C#N)nc2c1] |
| 419 | 2-(((1R)-1-(2-cyano-7-methyl-3- (methyl(1-methyl-2,3-dihydro-1H- inden-2-yl)amino)quinoxalin-5-yl)- ethyl)amino)benzoic acid | 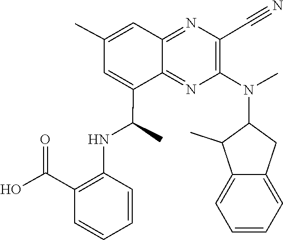 [SMILES: Cc1cc([C@@H](C)Nc2ccccc2C(=O)O)c2nc(N(C)C3Cc4ccccc4C3C)c(C#N)nc2c1] |
| 420 | 2-(((1R)-1-(2-cyano-3-((2-methoxy- 2,3-dihydro-1H-inden-1-yl)(methyl)- amino)-7-methylquinoxalin-5-yl)- ethyl)amino)benzoic acid | 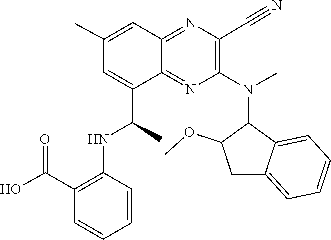 [SMILES: COC1Cc2ccccc2C1N(C)c1nc2c([C@@H](C)Nc3ccccc3C(=O)O)cc(C)cc2nc1C#N] |
| 421 | 2-(((1R)-1-(2-cyano-3-(3-(4-cyano- phenyl)-3,8-diazabicyclo[3.2.1]octan- 8-yl)-7-methylquinoxalin-5-yl)ethyl)- amino)benzoic acid | 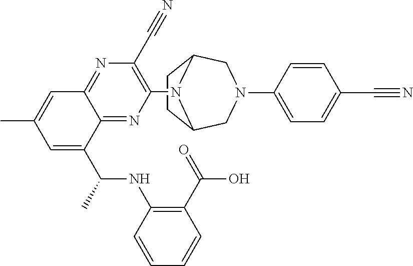 [SMILES: Cc1cc([C@@H](C)Nc2ccccc2C(=O)O)c2nc(N3C4CCC3CN(c3ccc(C#N)cc3)C4)c(C#N)nc2c1] |
| 422 | 2-(((1R)-1-(2-cyano-3-(3-(4-cyano- phenyl)-3,6-diazabicyclo[3.1.1]- heptan-6-yl)-7-methylquinoxalin-5- yl)ethyl)amino)benzoic acid | 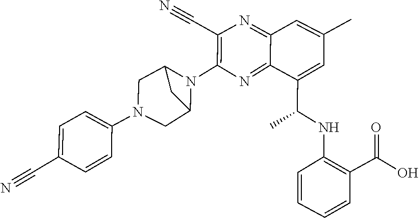 [SMILES: Cc1cc([C@@H](C)Nc2ccccc2C(=O)O)c2nc(N3C4CC3CN(c3ccc(C#N)cc3)C4)c(C#N)nc2c1] |
| 423 | (R)-2-((1-(2-cyano-7-methyl-3-(tetra- hydro-2H-pyran-4-yl)quinoxalin-5- yl)ethyl)amino)benzoic acid | 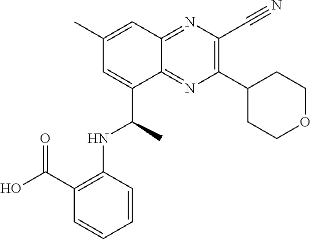 [SMILES: Cc1cc([C@@H](C)Nc2ccccc2C(=O)O)c2nc(C3CCOCC3)c(C#N)nc2c1] |
| 424 | (R)-2-((1-(2-cyano-7-methyl-3- (piperidin-4-yl)quinoxalin-5-yl)ethyl)- amino)benzoic acid | 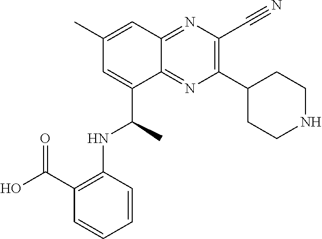 [SMILES: Cc1cc([C@@H](C)Nc2ccccc2C(=O)O)c2nc(C3CCNCC3)c(C#N)nc2c1] |
| 425 | (R)-2-((1-(2-cyano-7-methyl-3-(1- methylpiperidin-4-yl)quinoxalin-5- yl)ethyl)amino)benzoic acid | 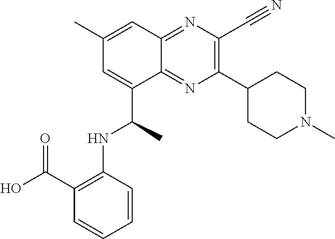 [SMILES: Cc1cc([C@@H](C)Nc2ccccc2C(=O)O)c2nc(C3CCN(C)CC3)c(C#N)nc2c1] |
| 426 | (R)-2-((1-(2-cyano-3-(3,6-dihydro- 2H-pyran-4-yl)-7-methylquinoxalin- 5-yl)ethyl)amino)benzoic acid | 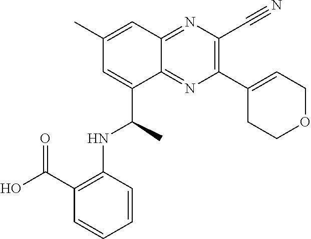 [SMILES: Cc1cc([C@@H](C)Nc2ccccc2C(=O)O)c2nc(C3=CCOCC3)c(C#N)nc2c1] |
| 427 | (R)-2-((1-(2-cyano-7-methyl-3- (1,2,3,6-tetrahydropyridin-4-yl)quin- oxalin-5-yl)ethyl)amino)benzoic acid | 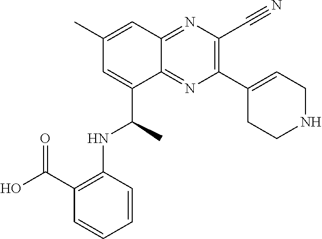 [SMILES: Cc1cc([C@@H](C)Nc2ccccc2C(=O)O)c2nc(C3=CCNCC3)c(C#N)nc2c1] |
| 428 | (R)-2-((1-(2-cyano-7-methyl-3-(1- methyl-1,2,3,6-tetrahydropyridin-4- yl)quinoxalin-5-yl)ethyl)amino)- benzoic acid | 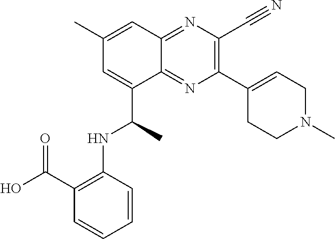 [SMILES: Cc1cc([C@@H](C)Nc2ccccc2C(=O)O)c2nc(C3=CCN(C)CC3)c(C#N)nc2c1] |
| 429 | (R)-2-((1-(2-cyano-3,7-dimethyl- quinoxalin-5-yl)ethyl)amino)benzoic acid | 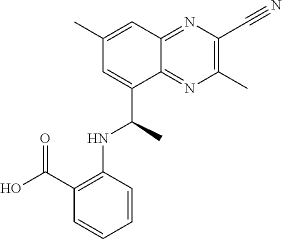 [SMILES: Cc1cc([C@@H](C)Nc2ccccc2C(=O)O)c2nc(C)c(C#N)nc2c1] |
| 430 | (R)-2-((1-(2-cyano-3-(1-(4-cyano- phenyl)piperidin-4-yl)-7-methylquin- oxalin-5-yl)ethyl)amino)benzoic acid | 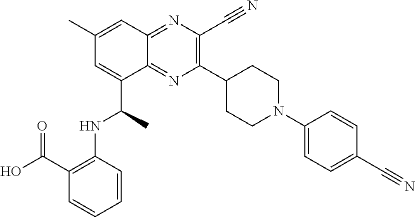 [SMILES: Cc1cc([C@@H](C)Nc2ccccc2C(=O)O)c2nc(C3CCN(c4ccc(C#N)cc4)CC3)c(C#N)nc2c1] |
| 431 | (R)-2-((1-(3-(1-acetylpiperidin-4-yl)- 2-cyano-7-methylquinoxalin-5-yl)- ethyl)amino)benzoic acid | 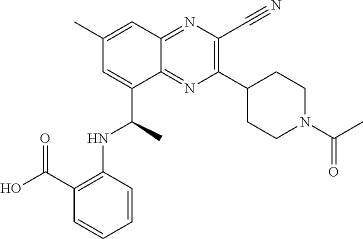 [SMILES: CC(=O)N1CCC(c2nc3c([C@@H](C)Nc4ccccc4C(=O)O)cc(C)cc3nc2C#N)CC1] |
| 432 | (R)-2-((1-(3-(1-acetyl-1,2,3,6-tetra- hydropyridin-4-yl)-2-cyano-7-methyl- quinoxalin-5-yl)ethyl)amino)benzoic acid | 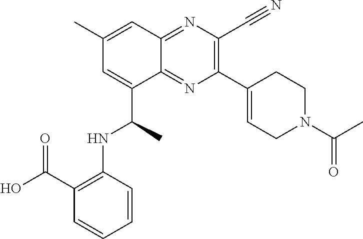 [SMILES: CC(=O)N1CC=C(c2nc3c([C@@H](C)Nc4ccccc4C(=O)O)cc(C)cc3nc2C#N)CC1] |
| 433 | (R)-2-((1-(2-cyano-3-((4-cyanophen- yl)ethynyl)-7-methylquinoxalin-5- yl)ethyl)amino)benzoic acid | 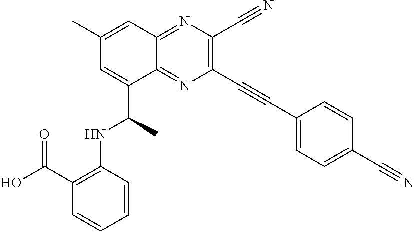 [SMILES: Cc1cc([C@@H](C)Nc2ccccc2C(=O)O)c2nc(C#Cc3ccc(C#N)cc3)c(C#N)nc2c1] |
| 434 | (R)-2-((1-(2-cyano-7-methyl-3-(1- methyl-1H-pyrazol-3-yl)quinoxalin- 5-yl)ethyl)amino)benzoic acid | 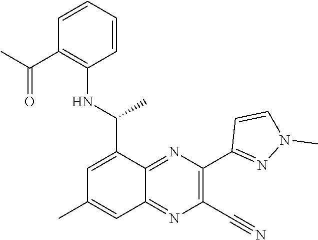 [SMILES: CC(=O)c1ccccc1N[C@H](C)c1cc(C)cc2nc(C#N)c(-c3ccn(C)n3)nc12] |
| 435 | (R)-2-((1-(2-cyano-7-methyl-3-(pyr- idin-2-yl)quinoxalin-5-yl)ethyl)- amino)benzoic acid | 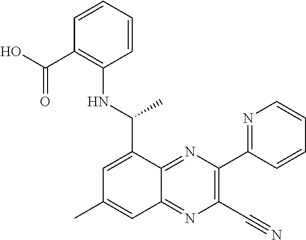 [SMILES: Cc1cc([C@@H](C)Nc2ccccc2C(=O)O)c2nc(-c3ccccn3)c(C#N)nc2c1] |
| 436 | (R)-2-((1-(2-cyano-7-methyl-3-(pyr- idin-3-yl)quinoxalin-5-yl)ethyl)- amino)benzoic acid | 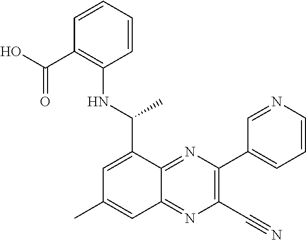 [SMILES: Cc1cc([C@@H](C)Nc2ccccc2C(=O)O)c2nc(-c3cccnc3)c(C#N)nc2c1] |
| 437 | (R)-2-((1-(2-cyano-7-methyl-3-(1- methyl-1H-pyrazol-4-yl)quinoxalin- 5-yl)ethyl)amino)benzoic acid | 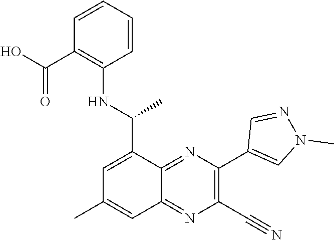 [SMILES: Cc1cc([C@@H](C)Nc2ccccc2C(=O)O)c2nc(-c3cnn(C)c3)c(C#N)nc2c1] |
| 438 | (R)-2-((1-(2-cyano-7-methyl-3-(1- methyl-1H-imidazol-4-yl)quinoxalin- 5-yl)ethyl)amino)benzoic acid | 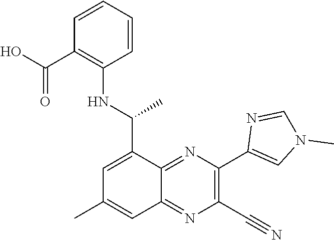 [SMILES: Cc1cc([C@@H](C)Nc2ccccc2C(=O)O)c2nc(-c3cn(C)cn3)c(C#N)nc2c1] |
| 439 | (R)-2-((1-(2-cyano-3-(1-(4-cyano- phenyl)-1H-pyrazol-4-yl)-7-methyl- quinoxalin-5-yl)ethyl)amino)benzoic acid | 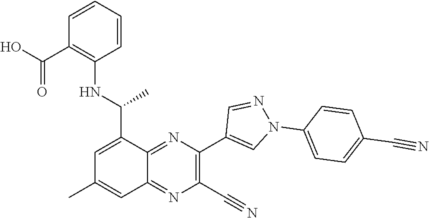 [SMILES: Cc1cc([C@@H](C)Nc2ccccc2C(=O)O)c2nc(-c3cnn(-c4ccc(C#N)cc4)c3)c(C#N)nc2c1] |
| 440 | (R)-2-((1-(2-cyano-7-methyl-3-(pyr- idin-4-yl)quinoxalin-5-yl)ethyl)- amino)benzoic acid | 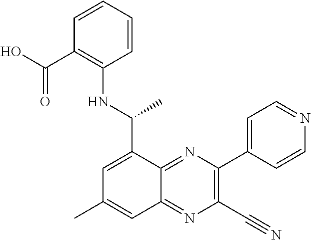 [SMILES: Cc1cc([C@@H](C)Nc2ccccc2C(=O)O)c2nc(-c3ccncc3)c(C#N)nc2c1] |
| 441 | (R)-2-((1-(2-cyano-7-methyl-3-phen- ylquinoxalin-5-yl)ethyl)amino)benzoic acid | 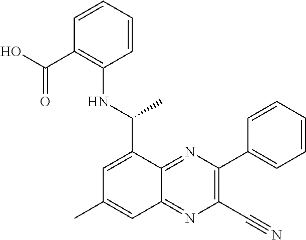 [SMILES: Cc1cc([C@@H](C)Nc2ccccc2C(=O)O)c2nc(-c3ccccc3)c(C#N)nc2c1] |
| 442 | (R)-2-((1-(2-cyano-7-methyl-3-(p- tolyl)quinoxalin-5-yl)ethyl)amino)- benzoic acid | 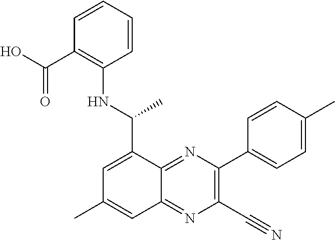 [SMILES: Cc1ccc(-c2nc3c([C@@H](C)Nc4ccccc4C(=O)O)cc(C)cc3nc2C#N)cc1] |
| 443 | (R)-2-((1-(2-cyano-3-(3-methoxy- phenyl)-7-methylquinoxalin-5-yl)- ethyl)amino)benzoic acid | 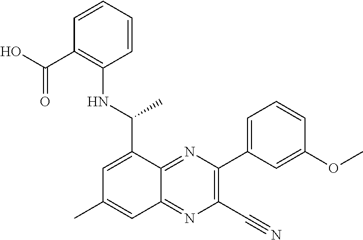 [SMILES: COc1cccc(-c2nc3c([C@@H](C)Nc4ccccc4C(=O)O)cc(C)cc3nc2C#N)c1] |
| 444 | (R)-2-((1-(2-cyano-3-(3-(2-cyano- propan-2-yl)phenyl)-7-methylquin- oxalin-5-yl)ethyl)amino)benzoic acid | 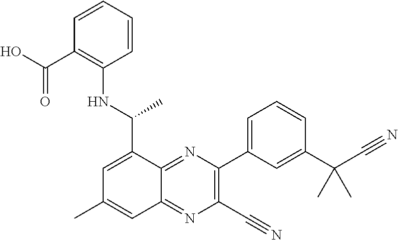 [SMILES: Cc1cc([C@@H](C)Nc2ccccc2C(=O)O)c2nc(-c3cccc(C(C)(C)C#N)c3)c(C#N)nc2c1] |
| 445 | (R)-2-((1-(2-cyano-3-(3-cyanophen- yl)-7-methylquinoxalin-5-yl)ethyl)- amino)benzoic acid | 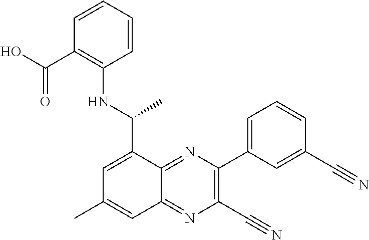 [SMILES: Cc1cc([C@@H](C)Nc2ccccc2C(=O)O)c2nc(-c3cccc(C#N)c3)c(C#N)nc2c1] |
| 446 | (R)-2-((1-(2-cyano-7-methyl-3-(1H- pyrazol-4-yl)quinoxalin-5-yl)ethyl)- amino)benzoic acid | 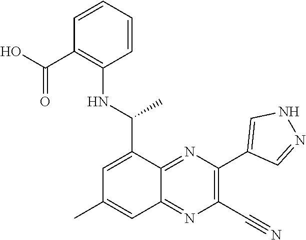 [SMILES: Cc1cc([C@@H](C)Nc2ccccc2C(=O)O)c2nc(-c3cn[nH]c3)c(C#N)nc2c1] |
| 447 | (R)-2-((1-(2-cyano-3-(1H-imidazol- 4-yl)-7-methylquinoxalin-5-yl)ethyl)- amino)benzoic acid | 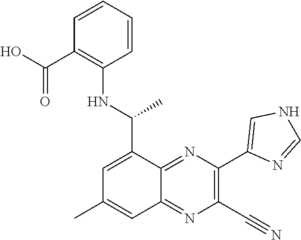 [SMILES: Cc1cc([C@@H](C)Nc2ccccc2C(=O)O)c2nc(-c3c[nH]cn3)c(C#N)nc2c1] |
| 448 | (R)-2-((1-(2-cyano-7-methyl-3-(o- tolyl)quinoxalin-5-yl)ethyl)amino)- benzoic acid | 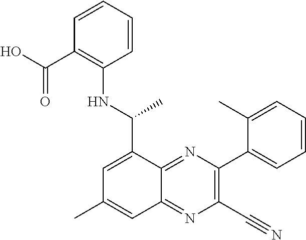 [SMILES: Cc1cc([C@@H](C)Nc2ccccc2C(=O)O)c2nc(-c3ccccc3C)c(C#N)nc2c1] |
| 449 | (R)-2-((1-(2-cyano-3-(1-(4-cyano- phenyl)-1H-imidazol-4-yl)-7-methyl- quinoxalin-5-yl)ethyl)amino)benzoic acid | 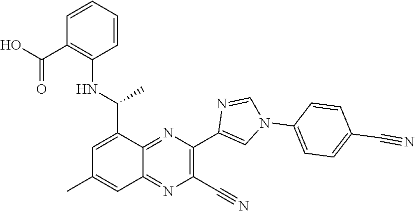 [SMILES: Cc1cc([C@@H](C)Nc2ccccc2C(=O)O)c2nc(-c3cn(-c4ccc(C#N)cc4)cn3)c(C#N)nc2c1] |
| 450 | (R)-2-((1-(2-cyano-3-(2-methoxy- phenyl)-7-methylquinoxalin-5-yl)- ethyl)amino)benzoic acid | 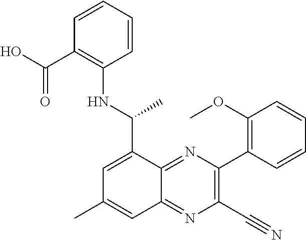 [SMILES: COc1ccccc1-c1nc2c([C@@H](C)Nc3ccccc3C(=O)O)cc(C)cc2nc1C#N] |
| 451 | (R)-2-((1-(2-cyano-3-(2-cyanophen- yl)-7-methylquinoxalin-5-yl)ethyl)- amino)benzoic acid | 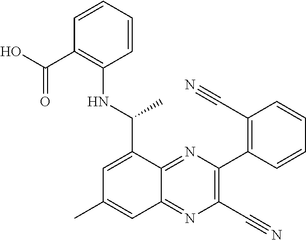 [SMILES: Cc1cc([C@@H](C)Nc2ccccc2C(=O)O)c2nc(-c3ccccc3C#N)c(C#N)nc2c1] |
| 452 | (R)-2-((1-(2-cyano-3-(4-methoxy- phenyl)-7-methylquinoxalin-5-yl)- ethyl)amino)benzoic acid | 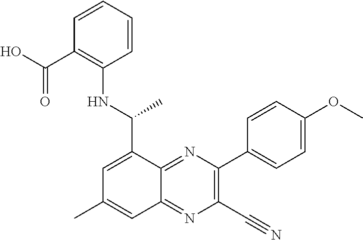 [SMILES: COc1ccc(-c2nc3c([C@@H](C)Nc4ccccc4C(=O)O)cc(C)cc3nc2C#N)cc1] |
| 453 | (R)-2-((1-(2-cyano-3-(4-cyanophen- yl)-7-methylquinoxalin-5-yl)ethyl)- amino)benzoic acid | 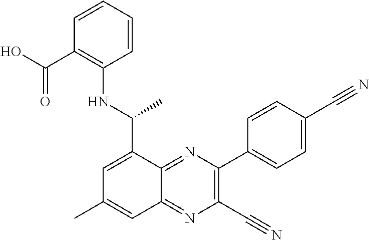 [SMILES: Cc1cc([C@@H](C)Nc2ccccc2C(=O)O)c2nc(-c3ccc(C#N)cc3)c(C#N)nc2c1] |
| 454 | 2-(((1R)-1-(2-cyano-7-methyl-3-(1- methyl-2-oxopiperidin-4-yl)quinox- alin-5-yl)ethyl)amino)benzoic acid | 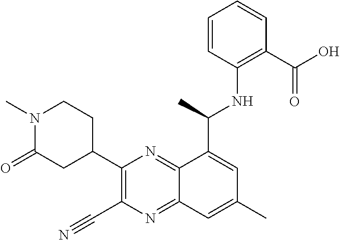 [SMILES: Cc1cc([C@@H](C)Nc2ccccc2C(=O)O)c2nc(C3CCN(C)C(=O)C3)c(C#N)nc2c1] |
| 455 | (R)-2-((1-(2-cyano-3-(4,4-difluoro- cyclohexyl)-7-methylquinoxalin-5- yl)ethyl)amino)benzoic acid | 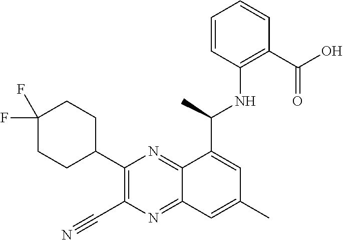 [SMILES: Cc1cc([C@@H](C)Nc2ccccc2C(=O)O)c2nc(C3CCC(F)(F)CC3)c(C#N)nc2c1] |
| 456 | (R)-2-((1-(2-cyano-7-methyl-3-(1- methyl-6-oxo-1,6-dihydropyridin-3- yl)quinoxalin-5-yl)ethyl)amino)- benzoic acid | 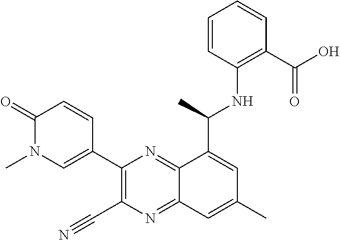 [SMILES: Cc1cc([C@@H](C)Nc2ccccc2C(=O)O)c2nc(-c3ccc(=O)n(C)c3)c(C#N)nc2c1] |
| 457 | (R)-2-((1-(2-cyano-3-(4-fluorophen- yl)-7-methylquinoxalin-5-yl)ethyl)- amino)benzoic acid | 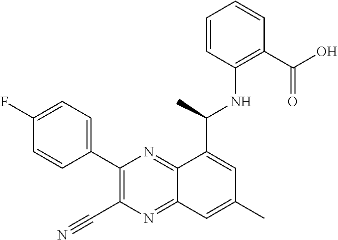 [SMILES: Cc1cc([C@@H](C)Nc2ccccc2C(=O)O)c2nc(-c3ccc(F)cc3)c(C#N)nc2c1] |
| 458 | (R)-2-((1-(3-(4-chlorophenyl)-2- cyano-7-methylquinoxalin-5-yl)- ethyl)amino)benzoic acid | 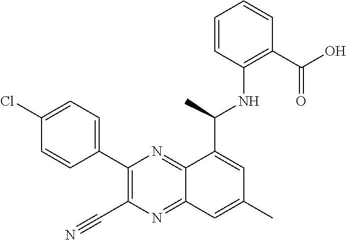 [SMILES: Cc1cc([C@@H](C)Nc2ccccc2C(=O)O)c2nc(-c3ccc(Cl)cc3)c(C#N)nc2c1] |
| 459 | 2-(((1R)-1-(2-cyano-7-methyl-3- ((tetrahydrofuran-3-yl)thio)quinox- alin-5-yl)ethyl)amino)benzoic acid | 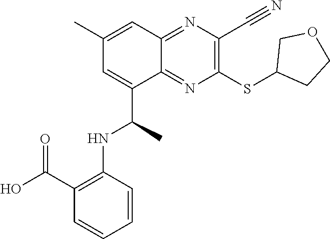 [SMILES: Cc1cc([C@@H](C)Nc2ccccc2C(=O)O)c2nc(SC3CCOC3)c(C#N)nc2c1] |
| 460 | (R)-2-((1-(2-cyano-7-methyl-3- (((trifluoromethyl)sulfonyl)oxy)- quinoxalin-5-yl)ethyl)amino)benzoic acid | 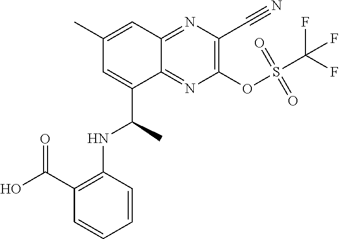 [SMILES: Cc1cc([C@@H](C)Nc2ccccc2C(=O)O)c2nc(OS(=O)(=O)C(F)(F)F)c(C#N)nc2c1] |
| 461 | (R)-2-((1-(2-cyano-3-(((1-fluoro- cyclopropyl)methyl)amino)-7-meth- ylquinoxalin-5-yl)ethyl)amino)- benzoic acid | 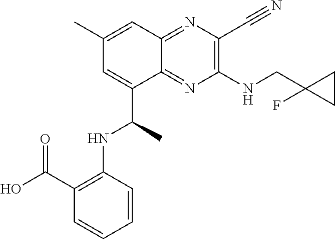 [SMILES: Cc1cc([C@@H](C)Nc2ccccc2C(=O)O)c2nc(NCC3(F)CC3)c(C#N)nc2c1] |
| 462 | (R)-2-((1-(2-cyano-7-methyl-3-(((1- (trifluoromethyl)cyclopropyl)meth- yl)amino)quinoxalin-5-yl)ethyl)- amino)benzoic acid | 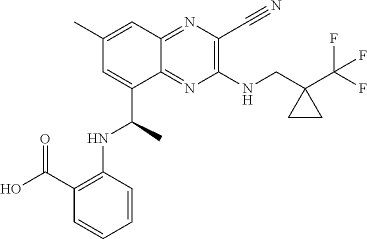 [SMILES: Cc1cc([C@@H](C)Nc2ccccc2C(=O)O)c2nc(NCC3(C(F)(F)F)CC3)c(C#N)nc2c1] |
| 463 | (R)-2-((1-(2-cyano-3-(((3,3-difluoro- cyclobutyl)methyl)amino)-7-methyl- quinoxalin-5-yl)ethyl)amino)benzoic acid | 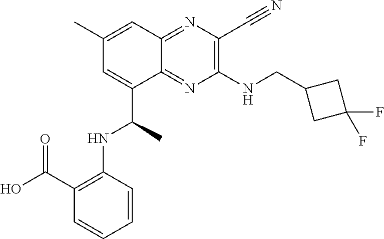 [SMILES: Cc1cc([C@@H](C)Nc2ccccc2C(=O)O)c2nc(NCC3CC(F)(F)C3)c(C#N)nc2c1] |
| 464 | 2-(((1R)-1-(2-cyano-3-(((2,2-difluoro- cyclopropyl)methyl)amino)-7-meth- ylquinoxalin-5-yl)ethyl)amino)benzoic acid | 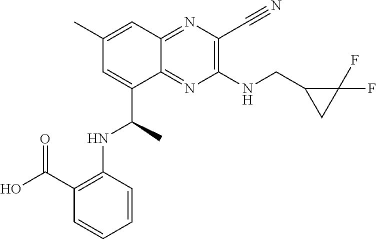 [SMILES: Cc1cc([C@@H](C)Nc2ccccc2C(=O)O)c2nc(NCC3CC3(F)F)c(C#N)nc2c1] |
| 465 | 2-(((1R)-1-(2-cyano-3-((1-cycloprop- yl-2,2,2-trifluoroethyl)amino)-7- methylquinoxalin-5-yl)ethyl)amino)- benzoic acid | 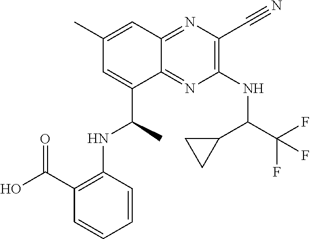 [SMILES: Cc1cc([C@@H](C)Nc2ccccc2C(=O)O)c2nc(NC(C3CC3)C(F)(F)F)c(C#N)nc2c1] |
| 466 | (R)-2-((1-(2-cyano-7-methyl-3-(3- (trifluoromethyl)-5,6-dihydro[1,2,4]- triazolo[4,3-a]pyrazin-7(8H)-yl)quin- oxalin-5-yl)ethyl)amino)benzoic acid | 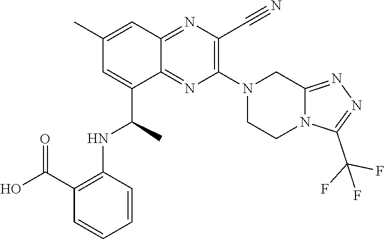 [SMILES: Cc1cc([C@@H](C)Nc2ccccc2C(=O)O)c2nc(N3CCn4c(nnc4C(F)(F)F)C3)c(C#N)nc2c1] |
| 467 | (R)-2-((1-(2-cyano-7-methyl-3-(2- (trifluoromethyl)-5,6-dihydroimidazo- [1,2-a]pyrazin-7(8H)-yl)quinoxalin- 5-yl)ethyl)amino)benzoic acid | 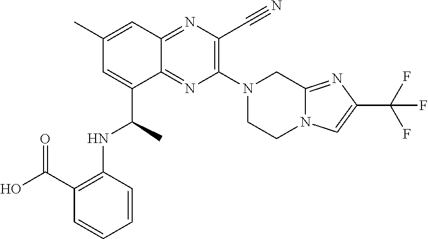 [SMILES: Cc1cc([C@@H](C)Nc2ccccc2C(=O)O)c2nc(N3CCn4cc(C(F)(F)F)nc4C3)c(C#N)nc2c1] |
| 468 | (R)-2-((1-(2-cyano-7-methyl-3-(4H- thieno[3,4-c]pyrrol-5(6H)-yl)quin- oxalin-5-yl)ethyl)amino)benzoic acid | 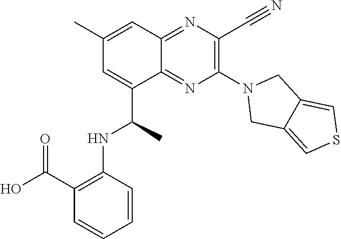 [SMILES: Cc1cc([C@@H](C)Nc2ccccc2C(=O)O)c2nc(N3Cc4cscc4C3)c(C#N)nc2c1] |
| 469 | (R)-2-((1-(2-cyano-3-(4-(2,2-difluoro- ethyl)piperazin-1-yl)-7-methylquin- oxalin-5-yl)ethyl)amino)benzoic acid | 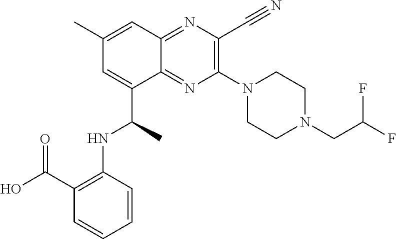 [SMILES: Cc1cc([C@@H](C)Nc2ccccc2C(=O)O)c2nc(N3CCN(CC(F)F)CC3)c(C#N)nc2c1] |
| 470 | (R)-2-((1-(2-cyano-7-methyl-3-(4- (2,2,2-trifluoroethyl)piperazin-1-yl)- quinoxalin-5-yl)ethyl)amino)benzoic acid | 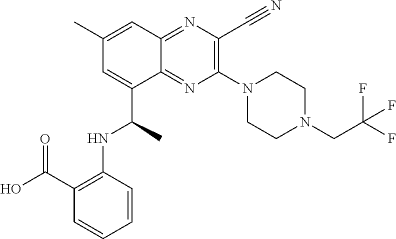 [SMILES: Cc1cc([C@@H](C)Nc2ccccc2C(=O)O)c2nc(N3CCN(CC(F)(F)F)CC3)c(C#N)nc2c1] |
| 471 | (R)-2-((1-(2-cyano-3-(2,2-difluoro-7- azaspiro[3.5]nonan-7-yl)-7-methyl- quinoxalin-5-yl)ethyl)amino)benzoic acid | 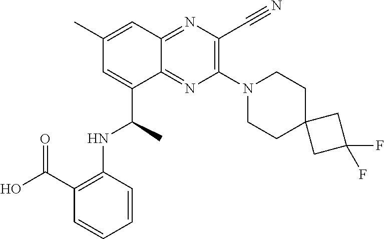 [SMILES: Cc1cc([C@@H](C)Nc2ccccc2C(=O)O)c2nc(N3CCC4(CC3)CC(F)(F)C4)c(C#N)nc2c1] |
| 472 | 2-(((1R)-1-(2-cyano-3-(2-(fluoro- methyl)morpholino)-7-methylquin- oxalin-5-yl)ethyl)amino)benzoic acid | 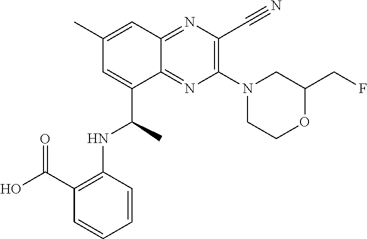 [SMILES: Cc1cc([C@@H](C)Nc2ccccc2C(=O)O)c2nc(N3CCOC(CF)C3)c(C#N)nc2c1] |
| 473 | 2-(((1R)-1-(2-cyano-3-(2-(2-fluoro- ethyl)morpholino)-7-methylquin- oxalin-5-yl)ethyl)amino)benzoic acid | 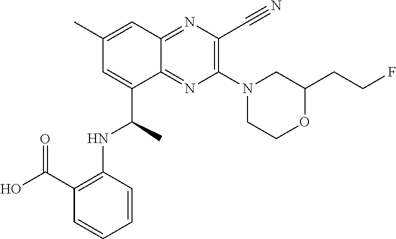 [SMILES: Cc1cc([C@@H](C)Nc2ccccc2C(=O)O)c2nc(N3CCOC(CCF)C3)c(C#N)nc2c1] |
| 474 | 2-(((1R)-1-(2-cyano-3-(5,5-difluoro- hexahydrocyclopenta[c]pyrrol-2(1H)- yl)-7-methylquinoxalin-5-yl)ethyl)- amino)benzoic acid | 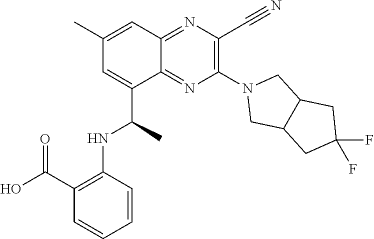 [SMILES: Cc1cc([C@@H](C)Nc2ccccc2C(=O)O)c2nc(N3CC4CC(F)(F)CC4C3)c(C#N)nc2c1] |
| 475 | (R)-2-((1-(2-cyano-3-(2,2-difluoro- 6-azaspiro[3.4]octan-6-yl)-7-methyl- quinoxalin-5-yl)ethyl)amino)benzoic acid | 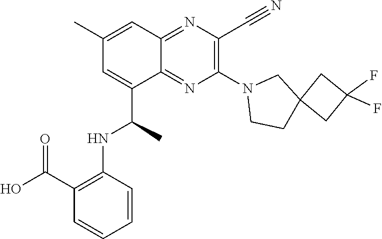 [SMILES: Cc1cc([C@@H](C)Nc2ccccc2C(=O)O)c2nc(N3CCC4(C3)CC(F)(F)C4)c(C#N)nc2c1] |
| 476 | (R)-2-((1-(2-cyano-3-(6,6-difluoro- 2-azaspiro[3.3]heptan-2-yl)-7-meth- ylquinoxalin-5-yl)ethyl)amino)- benzoic acid | 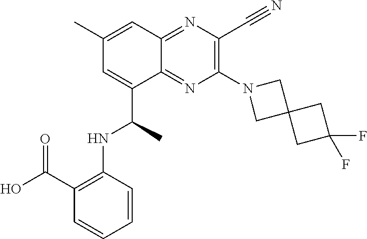 [SMILES: Cc1cc([C@@H](C)Nc2ccccc2C(=O)O)c2nc(N3CC4(C3)CC(F)(F)C4)c(C#N)nc2c1] |
| 477 | 2-(((1R)-1-(2-cyano-7-methyl-3-(2- (trifluoromethyl)morpholino)quin- oxalin-5-yl)ethyl)amino)benzoic acid | 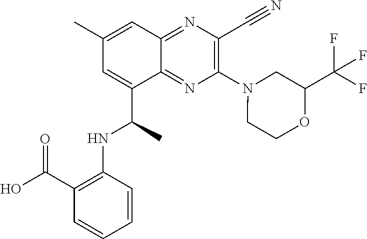 [SMILES: Cc1cc([C@@H](C)Nc2ccccc2C(=O)O)c2nc(N3CCOC(C(F)(F)F)C3)c(C#N)nc2c1] |
| 478 | 2-(((1R)-1-(2-cyano-3-(2-(fluoro- methyl)azetidin-1-yl)-7-methylquin- oxalin-5-yl)ethyl)amino)benzoic acid | 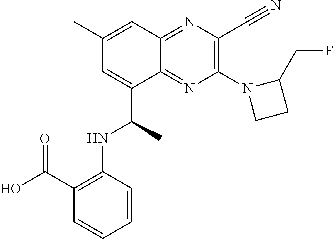 [SMILES: Cc1cc([C@@H](C)Nc2ccccc2C(=O)O)c2nc(N3CCC3CF)c(C#N)nc2c1] |
| 479 | 2-(((1R)-1-(2-cyano-3-(5-(2,2- difluoroethyl)-2,5-diazabicyclo[2.2.1]- heptan-2-yl)-7-methylquinoxalin-5- yl)ethyl)amino)benzoic acid | 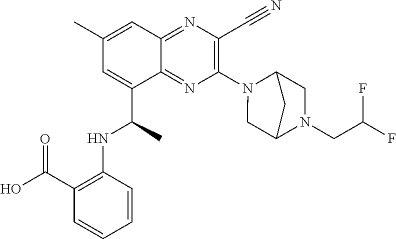 [SMILES: Cc1cc([C@@H](C)Nc2ccccc2C(=O)O)c2nc(N3CC4CC3CN4CC(F)F)c(C#N)nc2c1] |
| 480 | (R)-2-((1-(2-cyano-3-(4-fluoroiso- indolin-2-yl)-7-methylquinoxalin-5- yl)ethyl)amino)benzoic acid | 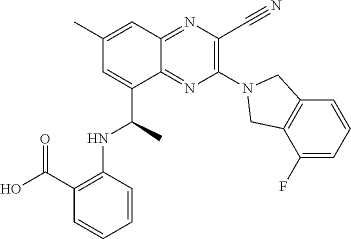 [SMILES: Cc1cc([C@@H](C)Nc2ccccc2C(=O)O)c2nc(N3Cc4cccc(F)c4C3)c(C#N)nc2c1] |
| 481 | (R)-2-((1-(2-cyano-7-methyl-3-(1- oxoisoindolin-2-yl)quinoxalin-5- yl)ethyl)amino)benzoic acid | 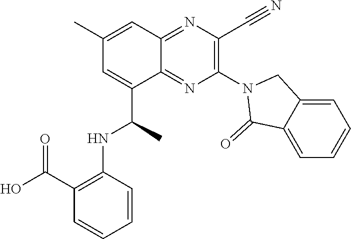 [SMILES: Cc1cc([C@@H](C)Nc2ccccc2C(=O)O)c2nc(N3Cc4ccccc4C3=O)c(C#N)nc2c1] |
| 482 | (R)-2-((1-(2-cyano-3-(5,6-difluoro- isoindolin-2-yl)-7-methylquinoxalin- 5-yl)ethyl)amino)benzoic acid | 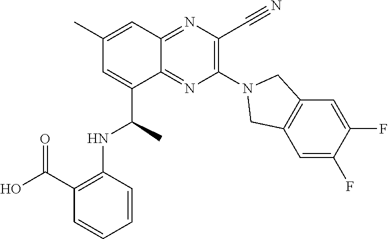 [SMILES: Cc1cc([C@@H](C)Nc2ccccc2C(=O)O)c2nc(N3Cc4cc(F)c(F)cc4C3)c(C#N)nc2c1] |
| 483 | (R)-2-((1-(2-cyano-7-methyl-3-(4- methyl-3-oxopiperazin-1-yl)quin- oxalin-5-yl)ethyl)amino)benzoic acid | 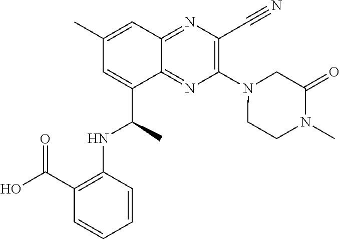 [SMILES: Cc1cc([C@@H](C)Nc2ccccc2C(=O)O)c2nc(N3CCN(C)C(=O)C3)c(C#N)nc2c1] |
| 484 | (R)-2-((1-(2-cyano-7-methyl-3-(3- oxopiperazin-1-yl)quinoxalin-5-yl)- ethyl)amino)benzoic acid | 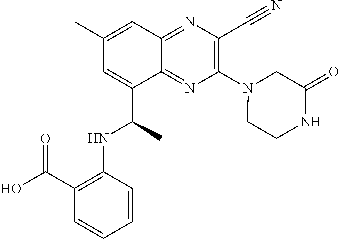 [SMILES: Cc1cc([C@@H](C)Nc2ccccc2C(=O)O)c2nc(N3CCNC(=O)C3)c(C#N)nc2c1] |
| 485 | 2-(((1R)-1-(2-cyano-3-(3,3-difluoro- 4-methoxypiperidin-1-yl)-7-meth- ylquinoxalin-5-yl)ethyl)amino)benzoic acid | 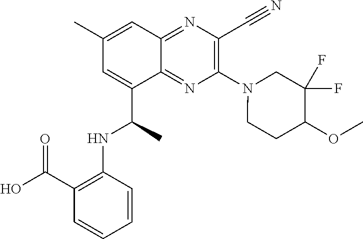 [SMILES: COC1CCN(c2nc3c([C@@H](C)Nc4ccccc4C(=O)O)cc(C)cc3nc2C#N)CC1(F)F] |
| 486 | 2-(((1R)-1-(2-cyano-3-(3,3-difluoro- 4-methylpiperidin-1-yl)-7-methyl- quinoxalin-5-yl)ethyl)amino)benzoic acid | 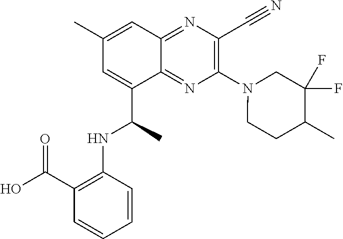 [SMILES: Cc1cc([C@@H](C)Nc2ccccc2C(=O)O)c2nc(N3CCC(C)C(F)(F)C3)c(C#N)nc2c1] |
| 487 | (R)-2-((1-(2-cyano-3-(4,4-difluoro- 6-azaspiro[2.5]octan-6-yl)-7-meth- ylquinoxalin-5-yl)ethyl)amino)- benzoic acid | 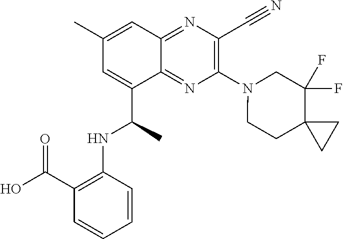 [SMILES: Cc1cc([C@@H](C)Nc2ccccc2C(=O)O)c2nc(N3CCC4(CC4)C(F)(F)C3)c(C#N)nc2c1] |
| 488 | 2-(((1R)-1-(2-cyano-7-methyl-3-(3- (trifluoromethyl)piperidin-1-yl)quin- oxalin-5-yl)ethyl)amino)benzoic acid | 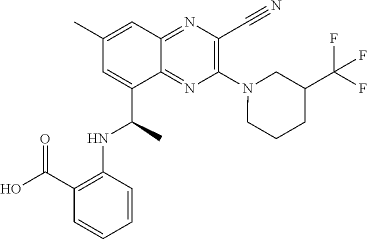 [SMILES: Cc1cc([C@@H](C)Nc2ccccc2C(=O)O)c2nc(N3CCCC(C(F)(F)F)C3)c(C#N)nc2c1] |
| 489 | (R)-2-((1-(2-cyano-3-(3,3-difluoro- piperidin-1-yl)-7-methylquinoxalin- 5-yl)ethyl)amino)benzoic acid | 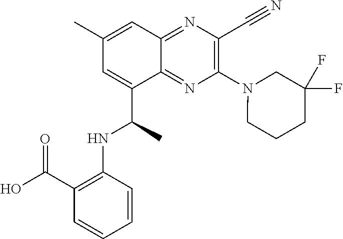 [SMILES: Cc1cc([C@@H](C)Nc2ccccc2C(=O)O)c2nc(N3CCCC(F)(F)C3)c(C#N)nc2c1] |
| 490 | 2-(((1R)-1-(2-cyano-3-(4,4-difluoro- 3-methoxypiperidin-1-yl)-7-meth- ylquinoxalin-5-yl)ethyl)amino)benzoic acid | 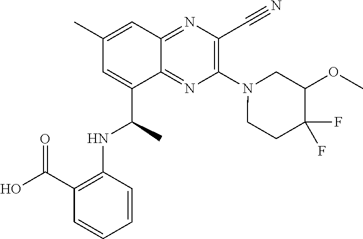 [SMILES: COC1CN(c2nc3c([C@@H](C)Nc4ccccc4C(=O)O)cc(C)cc3nc2C#N)CCC1(F)F] |
| 491 | 2-(((1R)-1-(2-cyano-7-methyl-3- (3,4,4-trifluoropiperidin-1-yl)quin- oxalin-5-yl)ethyl)amino)benzoic acid | 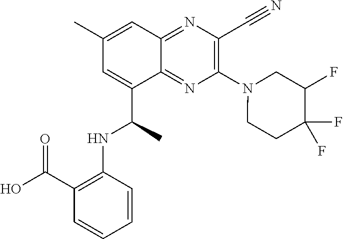 [SMILES: Cc1cc([C@@H](C)Nc2ccccc2C(=O)O)c2nc(N3CCC(F)(F)C(F)C3)c(C#N)nc2c1] |
| 492 | 2-(((1R)-1-(2-cyano-3-(3,3-difluoro- 4-methoxypyrrolidin-1-yl)-7-meth- ylquinoxalin-5-yl)ethyl)amino)benzoic acid | 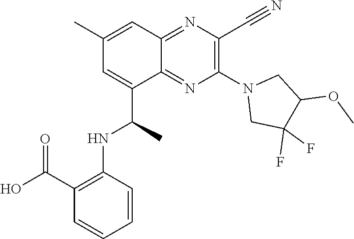 [SMILES: COC1CN(c2nc3c([C@@H](C)Nc4ccccc4C(=O)O)cc(C)cc3nc2C#N)CC1(F)F] |
| 493 | 2-(((1R)-1-(2-cyano-3-(9,9-difluoro- 3-oxa-7-azabicyclo[3.3.1]nonan-7- yl)-7-methylquinoxalin-5-yl)ethyl)- amino)benzoic acid | 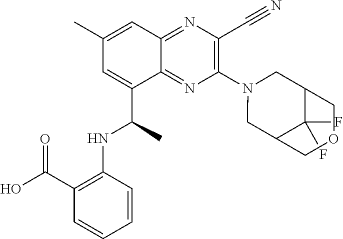 [SMILES: Cc1cc([C@@H](C)Nc2ccccc2C(=O)O)c2nc(N3CC4COCC(C3)C4(F)F)c(C#N)nc2c1] |
| 494 | 2-(((1R)-1-(2-cyano-3-(9,9-difluoro- 3-azabicyclo[3.3.1]nonan-3-yl)-7- methylquinoxalin-5-yl)ethyl)amino)- benzoic acid | 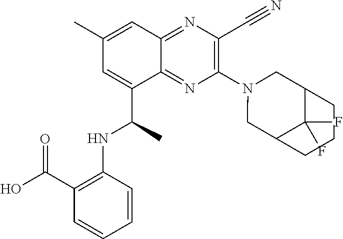 [SMILES: Cc1cc([C@@H](C)Nc2ccccc2C(=O)O)c2nc(N3CC4CCCC(C3)C4(F)F)c(C#N)nc2c1] |
| 495 | 2-(((1R)-1-(2-cyano-3-(9,9-difluoro- 1-(hydroxymethyl)-3-azabicyclo- [3.3.1]nonan-3-yl)-7-methylquin- oxalin-5-yl)ethyl)amino)benzoic acid | 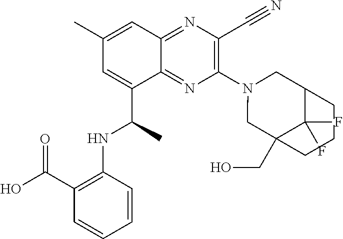 [SMILES: Cc1cc([C@@H](C)Nc2ccccc2C(=O)O)c2nc(N3CC4CCCC(CO)(C3)C4(F)F)c(C#N)nc2c1] |
| 496 | 2-(((1R)-1-(2-cyano-3-(8,8-difluoro- 3-azabicyclo[3.2.1]octan-3-yl)-7- methylquinoxalin-5-yl)ethyl)amino)- benzoic acid | 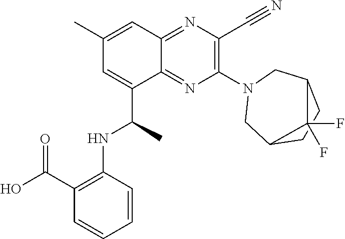 [SMILES: Cc1cc([C@@H](C)Nc2ccccc2C(=O)O)c2nc(N3CC4CCC(C3)C4(F)F)c(C#N)nc2c1] |
| 497 | 2-(((1R)-1-(2-cyano-3-(5,5-difluoro- 2-azabicyclo[2.2.2]octan-2-yl)-7- methylquinoxalin-5-yl)ethyl)amino)- benzoic acid | 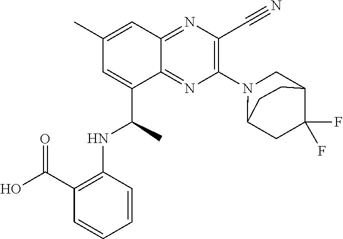 [SMILES: Cc1cc([C@@H](C)Nc2ccccc2C(=O)O)c2nc(N3CC4CCC3CC4(F)F)c(C#N)nc2c1] |
| 498 | 2-(((1R)-1-(2-cyano-7-methyl-3- (7,7,9,9-tetrafluoro-3-azabicyclo- [3.3.1]nonan-3-yl)quinoxalin-5-yl)- ethyl)amino)benzoic acid | 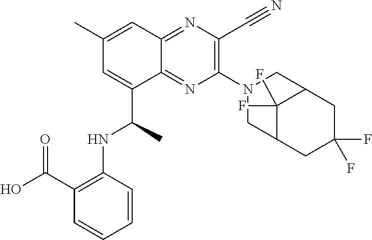 [SMILES: Cc1cc([C@@H](C)Nc2ccccc2C(=O)O)c2nc(N3CC4CC(F)(F)CC(C3)C4(F)F)c(C#N)nc2c1] |
| 499 | 2-(((1R)-1-(2-cyano-3-(6,6-difluoro- 3-azabicyclo[3.1.1]heptan-3-yl)-7- methylquinoxalin-5-yl)ethyl)amino)- benzoic acid | 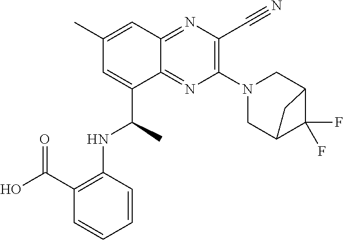 [SMILES: Cc1cc([C@@H](C)Nc2ccccc2C(=O)O)c2nc(N3CC4CC(C3)C4(F)F)c(C#N)nc2c1] |
| 500 | 2-(((1R)-1-(2-cyano-7-methyl-3-(1- (trifluoromethyl)-3-azabicyclo[3.1.0]- hexan-3-yl)quinoxalin-5-yl)ethyl)- amino)-benzoic acid | 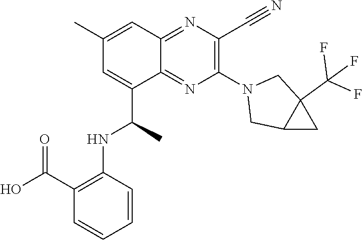 [SMILES: Cc1cc([C@@H](C)Nc2ccccc2C(=O)O)c2nc(N3CC4CC4(C(F)(F)F)C3)c(C#N)nc2c1] |
| 501 | 2-(((1R)-1-(2-cyano-3-(5,5-difluoro- 2-azabicyclo[2.2.1]heptan-2-yl)-7- methylquinoxalin-5-yl)ethyl)amino)- benzoic acid | 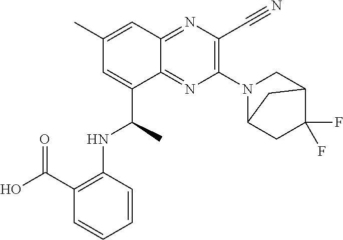 [SMILES: Cc1cc([C@@H](C)Nc2ccccc2C(=O)O)c2nc(N3CC4CC3CC4(F)F)c(C#N)nc2c1] |
| 502 | 2-(((1R)-1-(2-cyano-3-(4,4-difluoro- 3,5-dimethylpiperidin-1-yl)-7-methyl- quinoxalin-5-yl)ethyl)amino)benzoic acid | 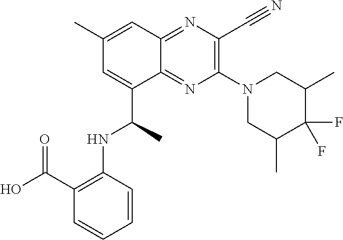 [SMILES: Cc1cc([C@@H](C)Nc2ccccc2C(=O)O)c2nc(N3CC(C)C(F)(F)C(C)C3)c(C#N)nc2c1] |
| 503 | (R)-2-((1-(2-cyano-3-(7,7-difluoro- 5-azaspiro[2.4]heptan-5-yl)-7-meth- ylquinoxalin-5-yl)ethyl)amino)- benzoic acid | 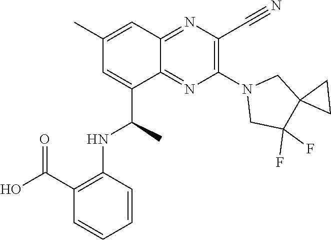 [SMILES: Cc1cc([C@@H](C)Nc2ccccc2C(=O)O)c2nc(N3CC(F)(F)C4(CC4)C3)c(C#N)nc2c1] |
| 504 | 2-(((1R)-1-(2-cyano-7-methyl-3-(6- (trifluoromethyl)-3-azabicyclo[3.1.0]- hexan-3-yl)quinoxalin-5-yl)ethyl)- amino)benzoic acid | 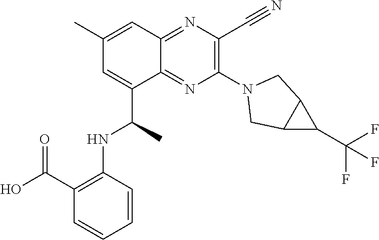 [SMILES: Cc1cc([C@@H](C)Nc2ccccc2C(=O)O)c2nc(N3CC4C(C3)C4C(F)(F)F)c(C#N)nc2c1] |
| 505 | 2-(((1R)-1-(2-cyano-3-(6,6-difluoro- 3-azabicyclo[3.1.0]hexan-3-yl)-7- methylquinoxalin-5-yl)ethyl)amino)- benzoic acid | 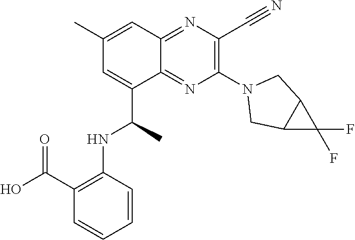 [SMILES: Cc1cc([C@@H](C)Nc2ccccc2C(=O)O)c2nc(N3CC4C(C3)C4(F)F)c(C#N)nc2c1] |
| 506 | (R)-2-((1-(2-cyano-3-(6,7-dihydro- thiazolo[4,5-c]pyridin-5(4H)-yl)-7- methylquinoxalin-5-yl)ethyl)amino)- benzoic acid | 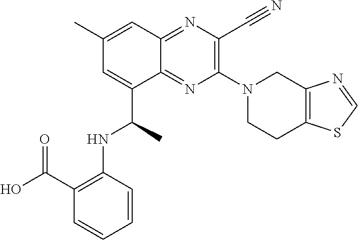 [SMILES: Cc1cc([C@@H](C)Nc2ccccc2C(=O)O)c2nc(N3CCc4scnc4C3)c(C#N)nc2c1] |
| 507 | (R)-2-((1-(2-cyano-7-methyl-3-(2- methyl-6,7-dihydrothiazolo[4,5-c]- pyridin-5(4H)-yl)quinoxalin-5-yl)- ethyl)amino)benzoic acid | 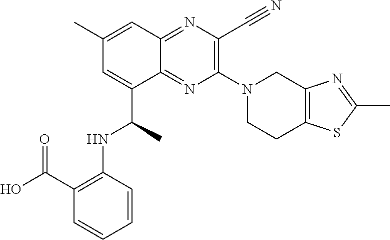 [SMILES: Cc1cc([C@@H](C)Nc2ccccc2C(=O)O)c2nc(N3CCc4sc(C)nc4C3)c(C#N)nc2c1] |
| 508 | (R)-2-((1-(2-cyano-3-(1,1-dioxido- thiomorpholino)-7-methylquinoxalin- 5-yl)ethyl)amino)benzoic acid | 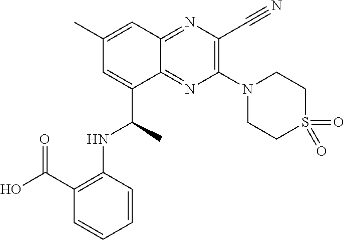 [SMILES: Cc1cc([C@@H](C)Nc2ccccc2C(=O)O)c2nc(N3CCS(=O)(=O)CC3)c(C#N)nc2c1] |
| 509 | (R)-2-((1-(2-cyano-7-methyl-3-(4- (thiazol-2-yl)piperazin-1-yl)quinox- alin-5-yl)ethyl)amino)benzoic acid | 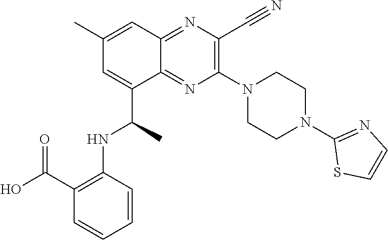 [SMILES: Cc1cc([C@@H](C)Nc2ccccc2C(=O)O)c2nc(N3CCN(c4nccs4)CC3)c(C#N)nc2c1] |
| 510 | (R)-2-((1-(2-cyano-7-methyl-3-(4- (5-(trifluoromethyl)pyridin-2-yl)- piperazin-1-yl)quinoxalin-5-yl)ethyl)- amino)benzoic acid | 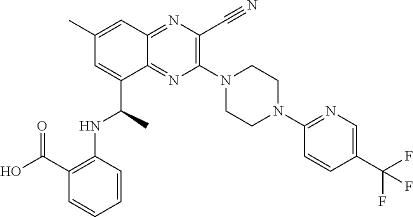 [SMILES: Cc1cc([C@@H](C)Nc2ccccc2C(=O)O)c2nc(N3CCN(c4ccc(C(F)(F)F)cn4)CC3)c(C#N)nc2c1] |
| 511 | (R)-2-((1-(2-cyano-7-methyl-3-(4-(5- (trifluoromethyl)benzo[d]thiazol-2- yl)piperazin-1-yl)quinoxalin-5-yl)- ethyl)amino)benzoic acid | 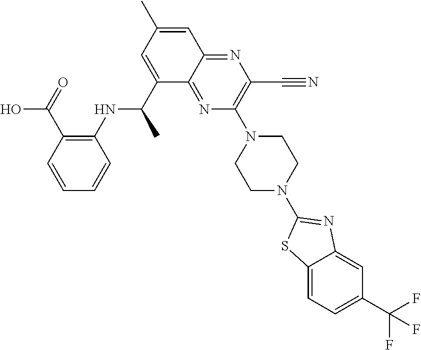 [SMILES: Cc1cc([C@@H](C)Nc2ccccc2C(=O)O)c2nc(N3CCN(c4nc5cc(C(F)(F)F)ccc5s4)CC3)c(C#N)nc2c1] |
| 512 | (R)-2-((1-(2-cyano-7-methyl-3-(4-(4- methylthiazol-2-yl)piperazin-1-yl)- quinoxalin-5-yl)ethyl)amino)benzoic acid | 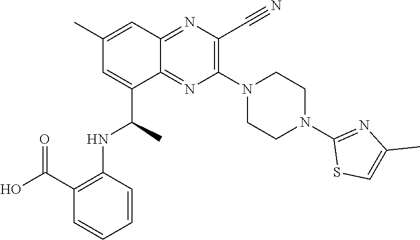 [SMILES: Cc1cc([C@@H](C)Nc2ccccc2C(=O)O)c2nc(N3CCN(c4nc(C)cs4)CC3)c(C#N)nc2c1] |
| 513 | (R)-2-((1-(2-cyano-7-methyl-3-(4-(1- methyl-3-(trifluoromethyl)-1H-pyr- azol-4-yl)piperazin-1-yl)quinoxalin- 5-yl)ethyl)amino)benzoic acid | 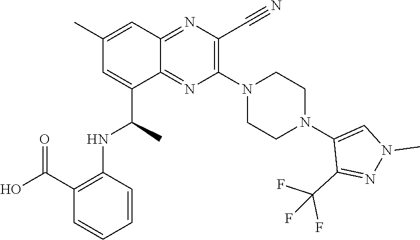 [SMILES: Cc1cc([C@@H](C)Nc2ccccc2C(=O)O)c2nc(N3CCN(c4cn(C)nc4C(F)(F)F)CC3)c(C#N)nc2c1] |
| 514 | (R)-2-((1-(2-cyano-7-methyl-3-(4-(3- (trifluoromethyl)phenyl)piperazin-1- yl)quinoxalin-5-yl)ethyl)amino)- benzoic acid | 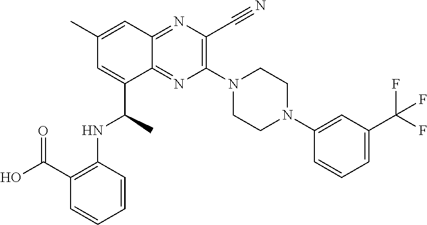 [SMILES: Cc1cc([C@@H](C)Nc2ccccc2C(=O)O)c2nc(N3CCN(c4cccc(C(F)(F)F)c4)CC3)c(C#N)nc2c1] |
| 515 | (R)-2-((1-(2-cyano-3-(4-(2-fluoro- phenyl)piperazin-1-yl)-7-methyl- quinoxalin-5-yl)ethyl)amino)benzoic acid | 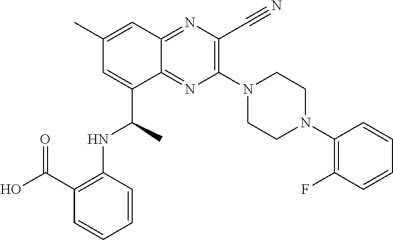 [SMILES: Cc1cc([C@@H](C)Nc2ccccc2C(=O)O)c2nc(N3CCN(c4ccccc4F)CC3)c(C#N)nc2c1] |
| 516 | (R)-2-((1-(3-(4-(2-chlorophenyl)- piperazin-1-yl)-2-cyano-7-methyl- quinoxalin-5-yl)ethyl)amino)benzoic acid | 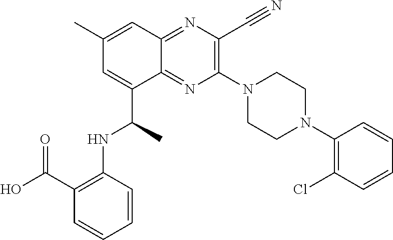 [SMILES: Cc1cc([C@@H](C)Nc2ccccc2C(=O)O)c2nc(N3CCN(c4ccccc4Cl)CC3)c(C#N)nc2c1] |
| 517 | (R)-2-((1-(2-cyano-3-(4-(2,4-difluoro- phenyl)piperazin-1-yl)-7-methyl- quinoxalin-5-yl)ethyl)amino)benzoic acid | 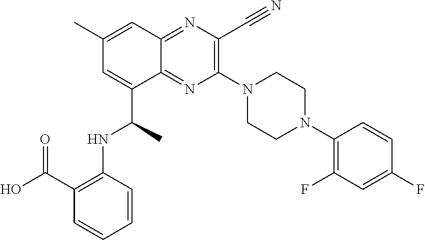 [SMILES: Cc1cc([C@@H](C)Nc2ccccc2C(=O)O)c2nc(N3CCN(c4ccc(F)cc4F)CC3)c(C#N)nc2c1] |
| 518 | (R)-2-((1-(2-cyano-3-(4-(4-cyano-2- fluorophenyl)piperazin-1-yl)-7- methylquinoxalin-5-yl)ethyl)amino)- benzoic acid | 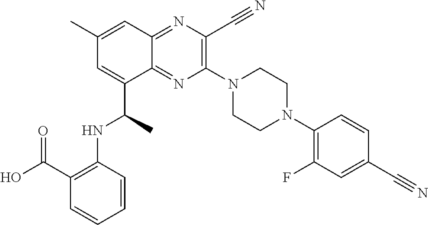 [SMILES: Cc1cc([C@@H](C)Nc2ccccc2C(=O)O)c2nc(N3CCN(c4ccc(C#N)cc4F)CC3)c(C#N)nc2c1] |
| 519 | (R)-2-((1-(2-cyano-7-methyl-3-(4- (4-(N-methylsulfamoyl)phenyl)piper- azin-1-yl)quinoxalin-5-yl)ethyl)- amino)benzoic acid | 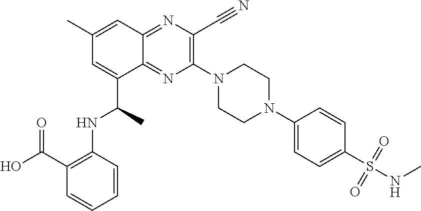 [SMILES: CNS(=O)(=O)c1ccc(N2CCN(c3nc4c([C@@H](C)Nc5ccccc5C(=O)O)cc(C)cc4nc3C#N)CC2)cc1] |
| 520 | (R)-2-((1-(2-cyano-3-(4-(4-fluoro- phenyl)piperazin-1-yl)-7-methylquin- oxalin-5-yl)ethyl)amino)benzoic acid | 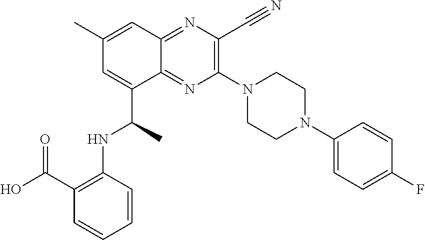 [SMILES: Cc1cc([C@@H](C)Nc2ccccc2C(=O)O)c2nc(N3CCN(c4ccc(F)cc4)CC3)c(C#N)nc2c1] |
| 521 | (R)-2-((1-(2-cyano-7-methyl-3-(4- (methylsulfonyl)piperazin-1-yl)quin- oxalin-5-yl)ethyl)amino)benzoic acid | 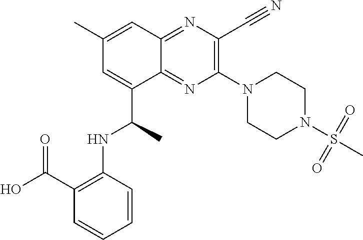 [SMILES: Cc1cc([C@@H](C)Nc2ccccc2C(=O)O)c2nc(N3CCN(S(C)(=O)=O)CC3)c(C#N)nc2c1] |
| 522 | (R)-2-((1-(3-(4-(4-chlorobenzoyl)- piperazin-1-yl)-2-cyano-7-methyl- quinoxalin-5-yl)ethyl)amino)benzoic acid | 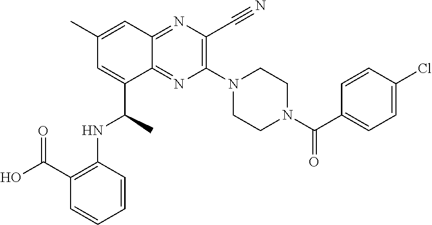 [SMILES: Cc1cc([C@@H](C)Nc2ccccc2C(=O)O)c2nc(N3CCN(C(=O)c4ccc(Cl)cc4)CC3)c(C#N)nc2c1] |
| 523 | (R)-2-((1-(2-cyano-7-methyl-3-(4-(3- (trifluoromethyl)-1H-pyrazol-1-yl)- piperidin-1-yl)quinoxalin-5-yl)ethyl)- amino)benzoic acid | 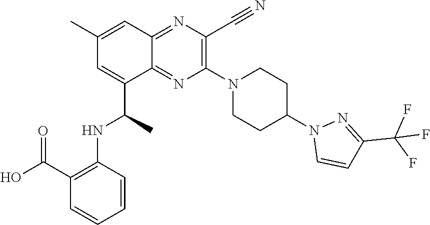 [SMILES: Cc1cc([C@@H](C)Nc2ccccc2C(=O)O)c2nc(N3CCC(n4ccc(C(F)(F)F)n4)CC3)c(C#N)nc2c1] |
| 524 | (R)-2-((1-(2-cyano-7-methyl-3-(2- oxo-[1,4′-bipiperidin]-1′-yl)quinox- alin-5-yl)ethyl)amino)benzoic acid | 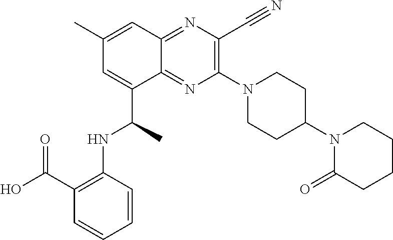 [SMILES: Cc1cc([C@@H](C)Nc2ccccc2C(=O)O)c2nc(N3CCC(N4CCCCC4=O)CC3)c(C#N)nc2c1] |
| 525 | (R)-2-((1-(2-cyano-3-(4,4-difluoro- [1,4′-bipiperidin]-1′-yl)-7-methyl- quinoxalin-5-yl)ethyl)amino)benzoic acid | 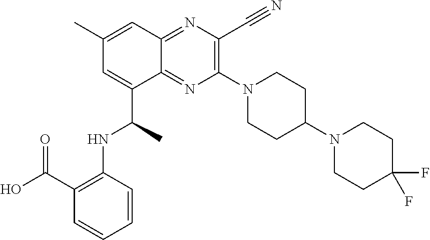 [SMILES: Cc1cc([C@@H](C)Nc2ccccc2C(=O)O)c2nc(N3CCC(N4CCC(F)(F)CC4)CC3)c(C#N)nc2c1] |
| 526 | (R)-2-((1-(2-cyano-7-methyl-3-(4- (trifluoromethyl)piperidin-1-yl)quin- oxalin-5-yl)ethyl)amino)benzoic acid | 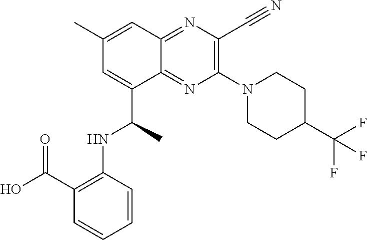 [SMILES: Cc1cc([C@@H](C)Nc2ccccc2C(=O)O)c2nc(N3CCC(C(F)(F)F)CC3)c(C#N)nc2c1] |
| 527 | (R)-2-((1-(2-cyano-3-(1,1-difluoro-6- azaspiro[2.5]octan-6-yl)-7-methyl- quinoxalin-5-yl)ethyl)amino)benzoic acid | 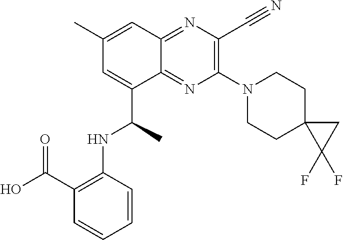 [SMILES: Cc1cc([C@@H](C)Nc2ccccc2C(=O)O)c2nc(N3CCC4(CC3)CC4(F)F)c(C#N)nc2c1] |
| 528 | 2-(((1R)-1-(2-cyano-7-methyl-3-(7- methyl-6-oxo-2,7-diazaspiro[4.4]- nonan-2-yl)quinoxalin-5-yl)ethyl)- amino)benzoic acid | 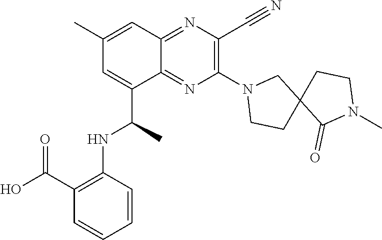 [SMILES: Cc1cc([C@@H](C)Nc2ccccc2C(=O)O)c2nc(N3CCC4(CCN(C)C4=O)C3)c(C#N)nc2c1] |
| 529 | 2-(((1R)-1-(2-cyano-7-methyl-3-(2- oxo-1,7-diazaspiro[4.4]nonan-7-yl)- quinoxalin-5-yl)ethyl)amino)benzoic acid | 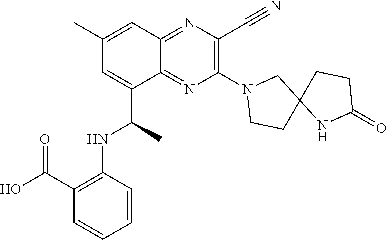 [SMILES: Cc1cc([C@@H](C)Nc2ccccc2C(=O)O)c2nc(N3CCC4(CCC(=O)N4)C3)c(C#N)nc2c1] |
| 530 | 2-(((1R)-1-(2-cyano-3-(7,7-difluoro- 2-azaspiro[4.4]nonan-2-yl)-7-meth- ylquinoxalin-5-yl)ethyl)amino)- benzoic acid | 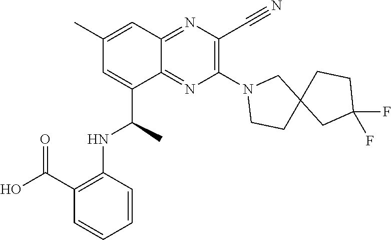 [SMILES: Cc1cc([C@@H](C)Nc2ccccc2C(=O)O)c2nc(N3CCC4(CCC(F)(F)C4)C3)c(C#N)nc2c1] |
| 531 | 2-(((1R)-1-(2-cyano-7-methyl-3- (1,1,2,2-tetrafluoro-6-azaspiro[3.4]- octan-6-yl)quinoxalin-5-yl)ethyl)- amino)benzoic acid | 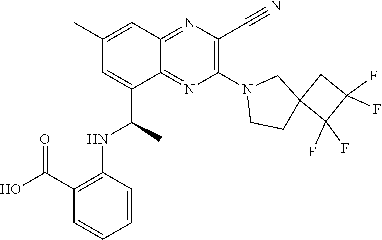 [SMILES: Cc1cc([C@@H](C)Nc2ccccc2C(=O)O)c2nc(N3CCC4(C3)CC(F)(F)C4(F)F)c(C#N)nc2c1] |
| 532 | 2-(((1R)-1-(2-cyano-3-(4,4-difluoro- 3-(hydroxymethyl)piperidin-1-yl)-7- methylquinoxalin-5-yl)ethyl)amino)- benzoic acid | 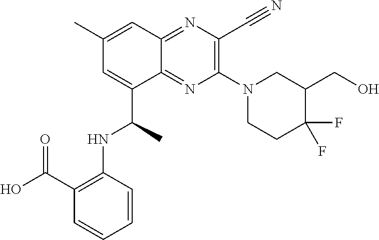 [SMILES: Cc1cc([C@@H](C)Nc2ccccc2C(=O)O)c2nc(N3CCC(F)(F)C(CO)C3)c(C#N)nc2c1] |
| 533 | 2-(((1R)-1-(2-cyano-7-methyl-3-(2- (trifluoromethyl)azetidin-1-yl)quin- oxalin-5-yl)ethyl)amino)benzoic acid | 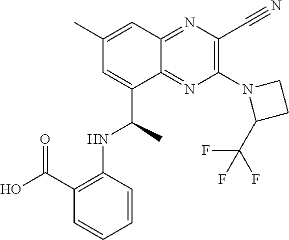 [SMILES: Cc1cc([C@@H](C)Nc2ccccc2C(=O)O)c2nc(N3CCC3C(F)(F)F)c(C#N)nc2c1] |
| 534 | 2-(((1R)-1-(2-cyano-3-(4-(2-cyano- 4-(trifluoromethyl)phenyl)-2-meth- ylpiperazin-1-yl)-7-methylquinoxalin- 5-yl)ethyl)amino)benzoic acid | 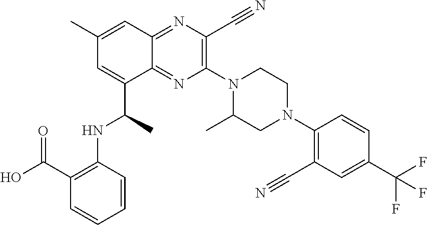 [SMILES: Cc1cc([C@@H](C)Nc2ccccc2C(=O)O)c2nc(N3CCN(c4ccc(C(F)(F)F)cc4C#N)CC3C)c(C#N)nc2c1] |
| 535 | 2-(((1R)-1-(2-cyano-3-(4-(4-fluoro- phenyl)-2-methylpiperazin-1-yl)-7- methylquinoxalin-5-yl)ethyl)amino)- benzoic acid | 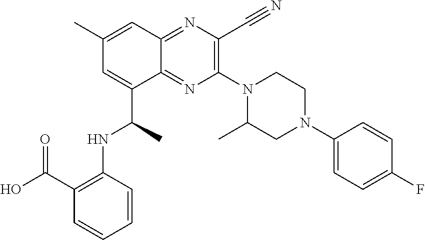 [SMILES: Cc1cc([C@@H](C)Nc2ccccc2C(=O)O)c2nc(N3CCN(c4ccc(F)cc4)CC3C)c(C#N)nc2c1] |
| 536 | 2-(((1R)-1-(2-cyano-3-(4,4-difluoro- 2-methylpiperidin-1-yl)-7-methyl- quinoxalin-5-yl)ethyl)amino)benzoic acid | 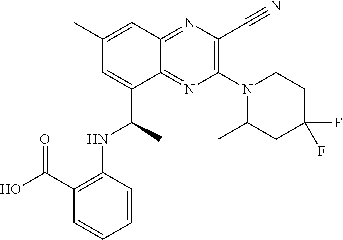 [SMILES: Cc1cc([C@@H](C)Nc2ccccc2C(=O)O)c2nc(N3CCC(F)(F)CC3C)c(C#N)nc2c1] |
| 537 | 2-(((1R)-1-(2-cyano-3-(3,3-difluoro- 2-methylpyrrolidin-1-yl)-7-methyl- quinoxalin-5-yl)ethyl)amino)benzoic acid | 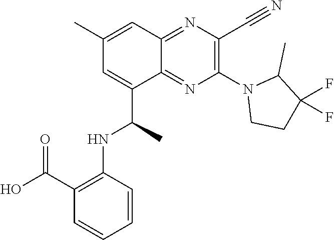 [SMILES: Cc1cc([C@@H](C)Nc2ccccc2C(=O)O)c2nc(N3CCC(F)(F)C3C)c(C#N)nc2c1] |
| 538 | (R)-2-((1-(2-cyano-7-methyl-3- (3,3,4,4-tetrafluoropyrrolidin-1-yl)- quinoxalin-5-yl)ethyl)amino)benzoic acid | 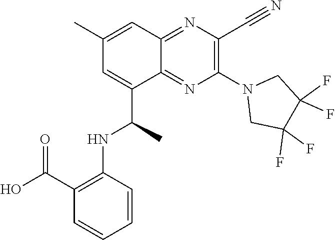 [SMILES: Cc1cc([C@@H](C)Nc2ccccc2C(=O)O)c2nc(N3CC(F)(F)C(F)(F)C3)c(C#N)nc2c1] |
| 539 | 2-(((1R)-1-(2-cyano-3-(3,5-difluoro- piperidin-1-yl)-7-methylquinoxalin- 5-yl)ethyl)amino)benzoic acid | 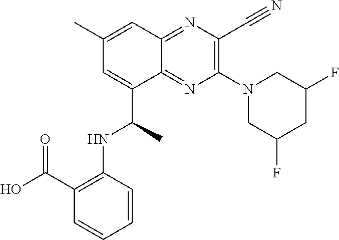 [SMILES: Cc1cc([C@@H](C)Nc2ccccc2C(=O)O)c2nc(N3CC(F)CC(F)C3)c(C#N)nc2c1] |
| 540 | 2-(((1R)-1-(2-cyano-3-(4-fluoro-2- (morpholine-4-carbonyl)pyrrolidin- 1-yl)-7-methylquinoxalin-5-yl)ethyl)- amino)benzoic acid | 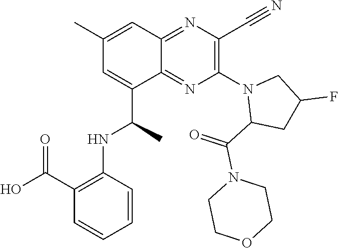 [SMILES: Cc1cc([C@@H](C)Nc2ccccc2C(=O)O)c2nc(N3CC(F)CC3C(=O)N3CCOCC3)c(C#N)nc2c1] |
| 541 | 2-(((1R)-1-(2-cyano-3-(6,6-difluoro- 2-azabicyclo[2.2.1]heptan-2-yl)-7- methylquinoxalin-5-yl)ethyl)amino)- benzoic acid | 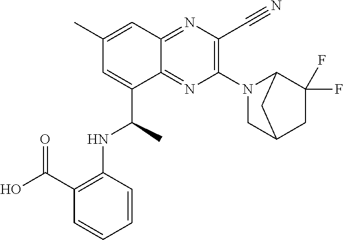 [SMILES: Cc1cc([C@@H](C)Nc2ccccc2C(=O)O)c2nc(N3CC4CC3C(F)(F)C4)c(C#N)nc2c1] |
| 542 | (R)-2-((1-(2-cyano-3-(4,6-dihydro- 5H-pyrrolo[3,4-d]thiazol-5-yl)-7- methylquinoxalin-5-yl)ethyl)amino)- benzoic acid | 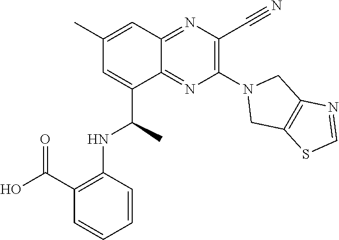 [SMILES: Cc1cc([C@@H](C)Nc2ccccc2C(=O)O)c2nc(N3Cc4ncsc4C3)c(C#N)nc2c1] |
| 543 | (R)-2-((1-(2-cyano-7-methyl-3-(2- methyl-4,6-dihydro-5H-pyrrolo[3,4- d]thiazol-5-yl)quinoxalin-5-yl)ethyl)- amino)benzoic acid | 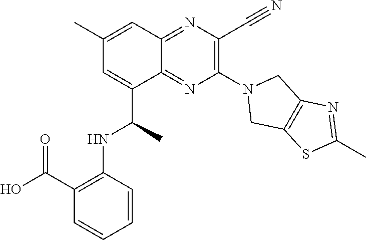 [SMILES: Cc1cc([C@@H](C)Nc2ccccc2C(=O)O)c2nc(N3Cc4nc(C)sc4C3)c(C#N)nc2c1] |
| 544 | (R)-2-((1-(2-cyano-3-(5-fluoroiso- indolin-2-yl)-7-methylquinoxalin- 5-yl)ethyl)amino)benzoic acid | 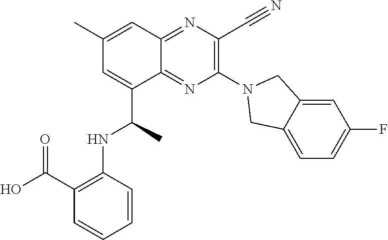 [SMILES: Cc1cc([C@@H](C)Nc2ccccc2C(=O)O)c2nc(N3Cc4ccc(F)cc4C3)c(C#N)nc2c1] |
| 545 | (R)-2-((1-(2-cyano-3-(5-(difluoro- methyl)isoindolin-2-yl)-7-methyl- quinoxalin-5-yl)ethyl)amino)benzoic acid | 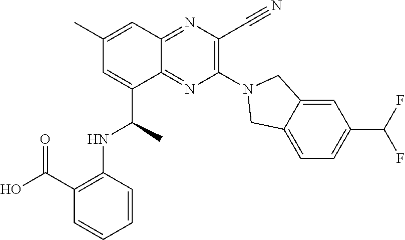 [SMILES: Cc1cc([C@@H](C)Nc2ccccc2C(=O)O)c2nc(N3Cc4ccc(C(F)F)cc4C3)c(C#N)nc2c1] |
| 546 | (R)-2-((1-(2-cyano-7-methyl-3-(3-(5- (trifluoromethyl)isoxazol-3-yl)azeti- din-1-yl)quinoxalin-5-yl)ethyl)- amino)benzoic acid | 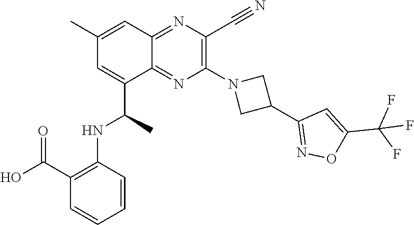 [SMILES: Cc1cc([C@@H](C)Nc2ccccc2C(=O)O)c2nc(N3CC(c4cc(C(F)(F)F)on4)C3)c(C#N)nc2c1] |
| 547 | (R)-2-((1-(2-cyano-3-(3-fluoroazeti- din-1-yl)-7-methylquinoxalin-5-yl)- ethyl)amino)benzoic acid | 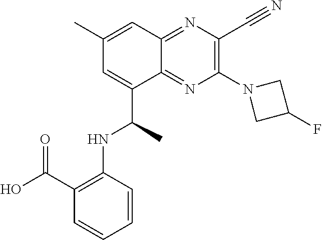 [SMILES: Cc1cc([C@@H](C)Nc2ccccc2C(=O)O)c2nc(N3CC(F)C3)c(C#N)nc2c1] |
| 548 | (R)-2-((1-(2-cyano-3-(3-(difluoro- methyl)azetidin-1-yl)-7-methylquin- oxalin-5-yl)ethyl)amino)benzoic acid | 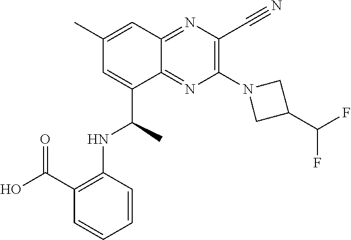 [SMILES: Cc1cc([C@@H](C)Nc2ccccc2C(=O)O)c2nc(N3CC(C(F)F)C3)c(C#N)nc2c1] |
| 549 | (R)-2-((1-(2-cyano-7-methyl-3-(3- (trifluoromethyl)azetidin-1-yl)quin- oxalin-5-yl)ethyl)amino)benzoic acid | 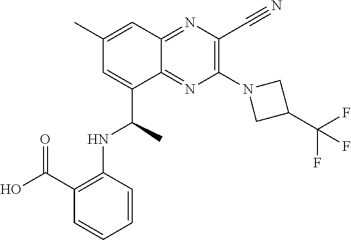 [SMILES: Cc1cc([C@@H](C)Nc2ccccc2C(=O)O)c2nc(N3CC(C(F)(F)F)C3)c(C#N)nc2c1] |
| 550 | (R)-2-((1-(2-cyano-3-(5,5-difluoro- 2-azaspiro[3.3]heptan-2-yl)-7-meth- ylquinoxalin-5-yl)ethyl)amino)- benzoic acid | 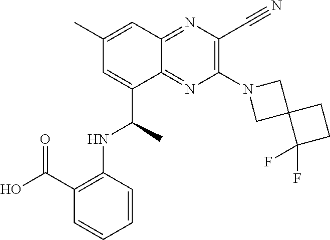 [SMILES: Cc1cc([C@@H](C)Nc2ccccc2C(=O)O)c2nc(N3CC4(CCC4(F)F)C3)c(C#N)nc2c1] |
| 551 | (R)-2-((1-(2-cyano-3-(1,1-difluoro- 5-azaspiro[2.3]hexan-5-yl)-7-meth- ylquinoxalin-5-yl)ethyl)amino)- benzoic acid | 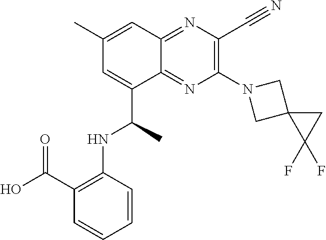 [SMILES: Cc1cc([C@@H](C)Nc2ccccc2C(=O)O)c2nc(N3CC4(C3)CC4(F)F)c(C#N)nc2c1] |
| 552 | 2-(((1R)-1-(3-(7-chloro-3-(hydroxy- methyl)-3,4-dihydroisoquinolin- 2(1H)-yl)-2-cyano-7-methylquin- oxalin-5-yl)ethyl)amino)benzoic acid | 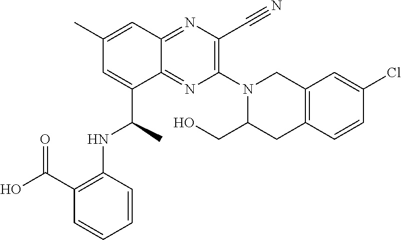 [SMILES: Cc1cc([C@@H](C)Nc2ccccc2C(=O)O)c2nc(N3Cc4cc(Cl)ccc4CC3CO)c(C#N)nc2c1] |
| 553 | 2-(((1R)-1-(2-cyano-3-(3,3-difluoro- 2-methylazetidin-1-yl)-7-methylquin- oxalin-5-yl)ethyl)amino)benzoic acid | 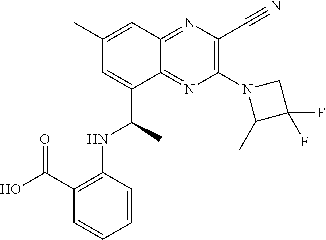 [SMILES: Cc1cc([C@@H](C)Nc2ccccc2C(=O)O)c2nc(N3CC(F)(F)C3C)c(C#N)nc2c1] |
| 554 | 2-(((1R)-1-(3-((1-benzyl-2-oxopiper- idin-3-yl)(methyl)amino)-2-cyano-7- methylquinoxalin-5-yl)ethyl)amino)- benzoic acid | 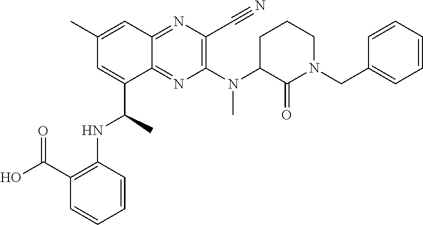 [SMILES: Cc1cc([C@@H](C)Nc2ccccc2C(=O)O)c2nc(N(C)C3CCCN(Cc4ccccc4)C3=O)c(C#N)nc2c1] |
| 555 | 2-(((1R)-1-(2-cyano-7-methyl-3- (methyl-(4,5,6,7-tetrahydrobenzo[d]- thiazol-6-yl)amino)quinoxalin-5-yl)- ethyl)amino)benzoic acid | 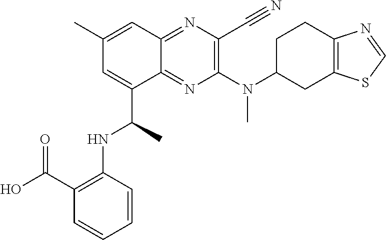 [SMILES: Cc1cc([C@@H](C)Nc2ccccc2C(=O)O)c2nc(N(C)C3CCc4ncsc4C3)c(C#N)nc2c1] |
| 556 | (R)-2-((1-(2-cyano-3-((3,3-difluoro- cyclobutyl)(methyl)amino)-7-methyl- quinoxalin-5-yl)ethyl)amino)benzoic acid | 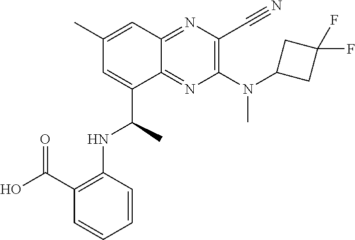 [SMILES: Cc1cc([C@@H](C)Nc2ccccc2C(=O)O)c2nc(N(C)C3CC(F)(F)C3)c(C#N)nc2c1] |
| 557 | 2-(((1R)-1-(2-cyano-7-methyl-3-(3- methyl-4-oxo-3,9-diazabicyclo- [4.2.1]nonan-9-yl)quinoxalin-5-yl)- ethyl)amino)benzoic acid | 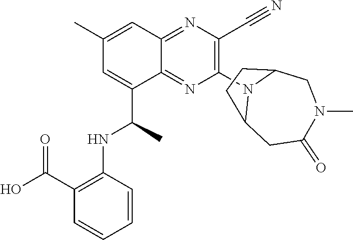 [SMILES: Cc1cc([C@@H](C)Nc2ccccc2C(=O)O)c2nc(N3C4CCC3CN(C)C(=O)C4)c(C#N)nc2c1] |
| 558 | 2-(((1R)-1-(2-cyano-3-(3,3-difluoro- 8-azabicyclo[3.2.1]octan-8-yl)-7- methylquinoxalin-5-yl)ethyl)amino)- benzoic acid | 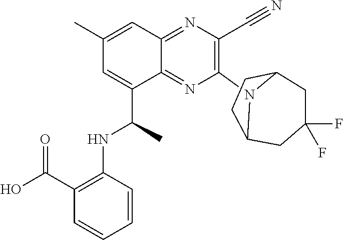 [SMILES: Cc1cc([C@@H](C)Nc2ccccc2C(=O)O)c2nc(N3C4CCC3CC(F)(F)C4)c(C#N)nc2c1] |
| 559 | (R)-2-((1-(3-chloro-2-cyano-7- methylquinoxalin-5-yl)ethyl)amino)- benzoic acid | 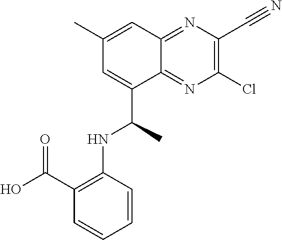 [SMILES: Cc1cc([C@@H](C)Nc2ccccc2C(=O)O)c2nc(Cl)c(C#N)nc2c1] |
| 560 | (R)-2-((1-(2-cyano-7-methyl-3-phen- oxyquinoxalin-5-yl)ethyl)amino)- benzoic acid | 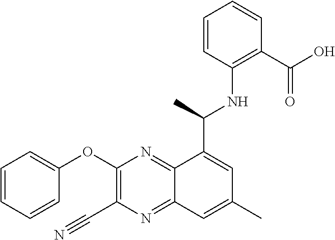 [SMILES: Cc1cc([C@@H](C)Nc2ccccc2C(=O)O)c2nc(Oc3ccccc3)c(C#N)nc2c1] |
| 561 | (R)-2-((1-(2-cyano-3-(3-cyanophen- oxy)-7-methylquinoxalin-5-yl)ethyl)- amino)benzoic acid | 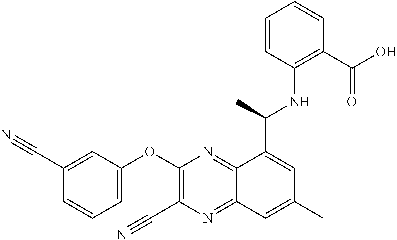 [SMILES: Cc1cc([C@@H](C)Nc2ccccc2C(=O)O)c2nc(Oc3cccc(C#N)c3)c(C#N)nc2c1] |
| 562 | (R)-2-((1-(3-(benzyloxy)-2-cyano-7- methylquinoxalin-5-yl)ethyl)amino)- benzoic acid | 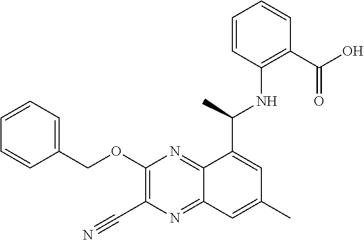 [SMILES: Cc1cc([C@@H](C)Nc2ccccc2C(=O)O)c2nc(OCc3ccccc3)c(C#N)nc2c1] |
| 563 | 2-(((1R)-1-(2-cyano-7-methyl-3- ((tetrahydrofuran-3-yl)oxy)quinox- alin-5-yl)ethyl)amino)benzoic acid | 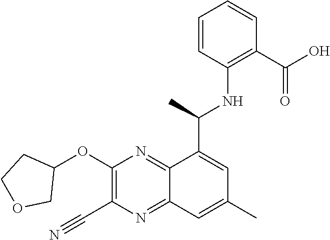 [SMILES: Cc1cc([C@@H](C)Nc2ccccc2C(=O)O)c2nc(OC3CCOC3)c(C#N)nc2c1] |
| 564 | (R)-2-((1-(2-cyano-3-methoxy-7- methylquinoxalin-5-yl)ethyl)amino)- benzoic acid | 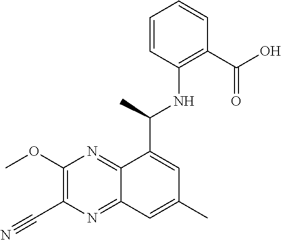 [SMILES: COc1nc2c([C@@H](C)Nc3ccccc3C(=O)O)cc(C)cc2nc1C#N] |
| 565 | (R)-2-((1-(2-cyano-3-(4-(ethoxy- methyl)piperidin-1-yl)-7-methylquin- oxalin-5-yl)ethyl)amino)benzoic acid | 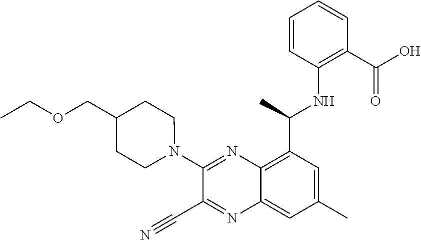 [SMILES: CCOCC1CCN(c2nc3c([C@@H](C)Nc4ccccc4C(=O)O)cc(C)cc3nc2C#N)CC1] |
| 566 | (R)-2-((1-(2-cyano-3-(diethylamino)- 7-methylquinoxalin-5-yl)ethyl)- amino)benzoic acid | 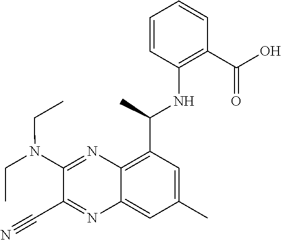 [SMILES: CCN(CC)c1nc2c([C@@H](C)Nc3ccccc3C(=O)O)cc(C)cc2nc1C#N] |
| 567 | 2-(((1R)-1-(2-cyano-7-methyl-3-((1- (pyridin-3-yl)ethyl)amino)quinox- alin-5-yl)ethyl)amino)benzoic acid | 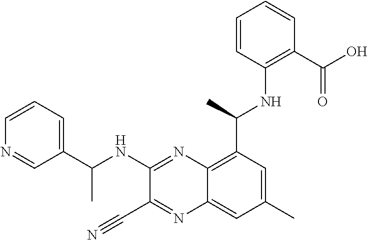 [SMILES: Cc1cc([C@@H](C)Nc2ccccc2C(=O)O)c2nc(NC(C)c3cccnc3)c(C#N)nc2c1] |
| 568 | 2-(((1R)-1-(2-cyano-7-methyl-3- ((3,3,3-trifluoro-2-methylpropyl)- amino)quinoxalin-5-yl)ethyl)amino)- benzoic acid | 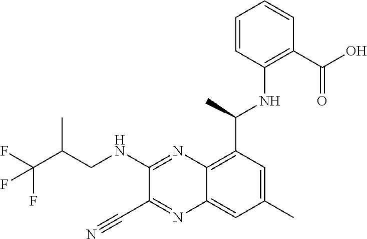 [SMILES: Cc1cc([C@@H](C)Nc2ccccc2C(=O)O)c2nc(NCC(C)C(F)(F)F)c(C#N)nc2c1] |
| 569 | 2-(((1R)-1-(2-cyano-3-((1-cyclo- propylethyl)amino)-7-methylquin- oxalin-5-yl)ethyl)amino)benzoic acid | 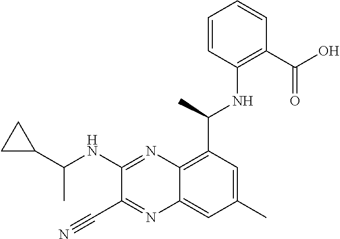 [SMILES: Cc1cc([C@@H](C)Nc2ccccc2C(=O)O)c2nc(NC(C)C3CC3)c(C#N)nc2c1] |
| 570 | (R)-2-((1-(2-cyano-3-(isobutyl- amino)-7-methylquinoxalin-5-yl)- ethyl)amino)benzoic acid | 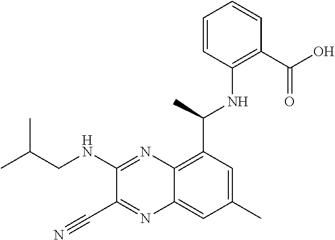 [SMILES: Cc1cc([C@@H](C)Nc2ccccc2C(=O)O)c2nc(NCC(C)C)c(C#N)nc2c1] |
| 571 | 2-(((1R)-1-(2-cyano-3-(3-hydroxy-3- isopropylpyrrolidin-1-yl)-7-methyl- quinoxalin-5-yl)ethyl)amino)benzoic acid | 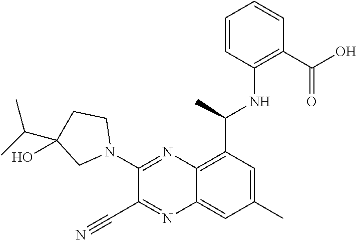 [SMILES: Cc1cc([C@@H](C)Nc2ccccc2C(=O)O)c2nc(N3CCC(O)(C(C)C)C3)c(C#N)nc2c1] |
| 572 | 2-(((1R)-1-(2-cyano-3-(1-isopropyl- 7,7-dioxido-7-thia-2-azaspiro[3.5]- nonan-2-yl)-7-methylquinoxalin-5- yl)ethyl)amino)benzoic acid | 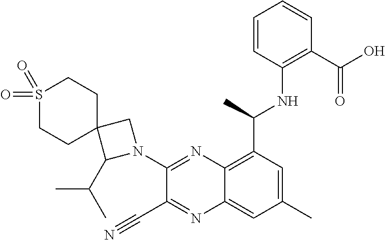 [SMILES: Cc1cc([C@@H](C)Nc2ccccc2C(=O)O)c2nc(N3CC4(CCS(=O)(=O)CC4)C3C(C)C)c(C#N)nc2c1] |

### Examples 573-846

The compounds in Table 3 are prepared essentially according to the procedures set forth in the above schemes and examples.

TABLE 3

| Example | | |
|---|---|---|
| No. | Name | Structure |
| 573 | (R)-3-((1-(3-(4-(bicyclo[1.1.1]pentan- 1-yl-3-d)piperidin-1-yl)-2-cyano-7- methylquinoxalin-5-yl)ethyl)amino)-6- chloropicolinic acid | 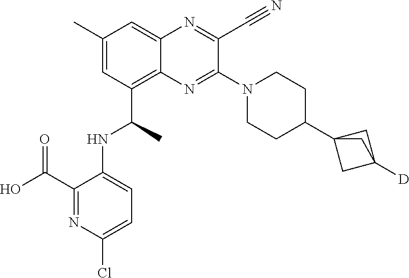 [SMILES: [2H]C12CC(C3CCN(c4nc5c([C@@H](C)Nc6ccc(Cl)nc6C(=O)O)cc(C)cc5nc4C#N)CC3)(C1)C2] |
| 574 | (R)-6-chloro-3-((1-(2-cyano-3-((3- cyanophenyl)amino)-7- methylquinoxalin-5- yl)ethyl)amino)picolinic acid | 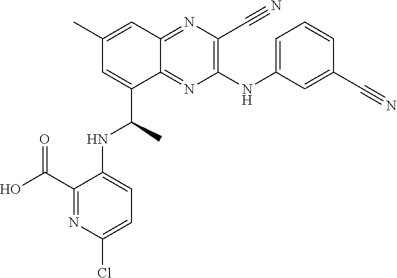 [SMILES: Cc1cc([C@@H](C)Nc2ccc(Cl)nc2C(=O)O)c2nc(Nc3cccc(C#N)c3)c(C#N)nc2c1] |
| 575 | 6-chloro-3-(((1R)-1-(2-cyano-7- methyl-3-(((tetrahydrofuran-3- yl)methyl)amino)quinoxalin-5- yl)ethyl)amino)picolinic acid | 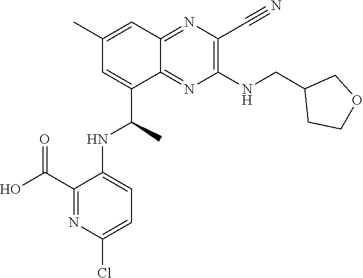 [SMILES: Cc1cc([C@@H](C)Nc2ccc(Cl)nc2C(=O)O)c2nc(NCC3CCOC3)c(C#N)nc2c1] |
| 576 | (R)-6-chloro-3-((1-(2-cyano-3-(((3,3- difluorocyclobutyl)methyl)amino)-7- methylquinoxalin-5- yl)ethyl)amino)picolinic acid | 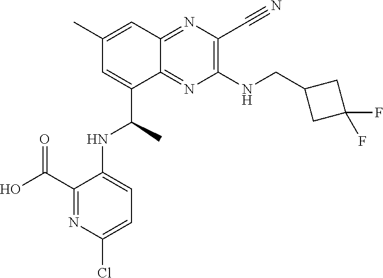 [SMILES: Cc1cc([C@@H](C)Nc2ccc(Cl)nc2C(=O)O)c2nc(NCC3CC(F)(F)C3)c(C#N)nc2c1] |
| 577 | (R)-6-chloro-3-((1-(2-cyano-3-((2- cyanoallyl)amino)-7- methylquinoxalin-5- yl)ethyl)amino)picolinic acid | 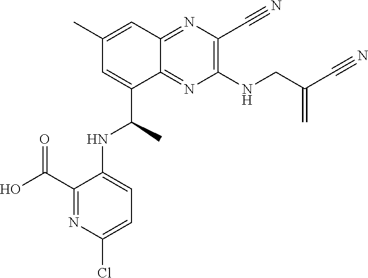 [SMILES: C=C(C#N)CNc1nc2c([C@@H](C)Nc3ccc(Cl)nc3C(=O)O)cc(C)cc2nc1C#N] |
| 578 | (R)-6-chloro-3-((1-(2-cyano-3- (cyclobutylamino)-7- methylquinoxalin-5- yl)ethyl)amino)picolinic acid | 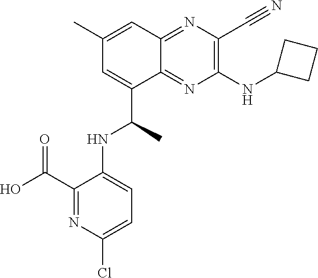 [SMILES: Cc1cc([C@@H](C)Nc2ccc(Cl)nc2C(=O)O)c2nc(NC3CCC3)c(C#N)nc2c1] |
| 579 | (R)-6-chloro-3-((1-(2-cyano-3-((3- (hydroxymethyl)bicyclo[1.1.1]pentan- 1-yl)amino)-7-methylquinoxalin-5- yl)ethyl)amino)picolinic acid | 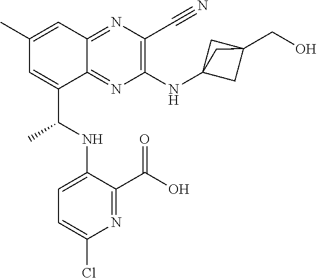 [SMILES: Cc1cc([C@@H](C)Nc2ccc(Cl)nc2C(=O)O)c2nc(NC34CC(CO)(C3)C4)c(C#N)nc2c1] |
| 580 | 6-chloro-3-(((1R)-1-(2-cyano-3-((1- cyclopropyl-2,2,2- trifluoroethyl)amino)-7- methylquinoxalin-5- yl)ethyl)amino)picolinic acid | 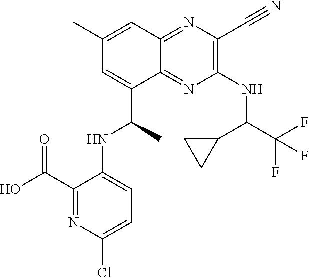 [SMILES: Cc1cc([C@@H](C)Nc2ccc(Cl)nc2C(=O)O)c2nc(NC(C3CC3)C(F)(F)F)c(C#N)nc2c1] |
| 581 | (R)-6-chloro-3-((1-(2-cyano-7-methyl- 3-(3-(trifluoromethyl)-5,6-dihydro- [1,2,4]triazolo[4,3-a]pyrazin-7(8H)- yl)quinoxalin-5- yl)ethyl)amino)picolinic acid | 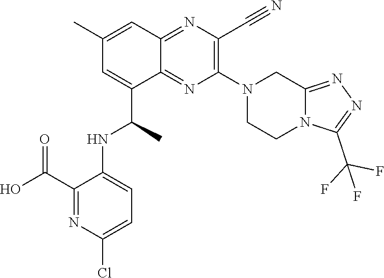 [SMILES: Cc1cc([C@@H](C)Nc2ccc(Cl)nc2C(=O)O)c2nc(N3CCn4c(nnc4C(F)(F)F)C3)c(C#N)nc2c1] |
| 582 | (R)-6-chloro-3-((1-(2-cyano-3-(5,6- dihydroimidazo[1,2-a]pyrazin-7(8H)- yl)-7-methylquinoxalin-5- yl)ethyl)amino)picolinic acid | 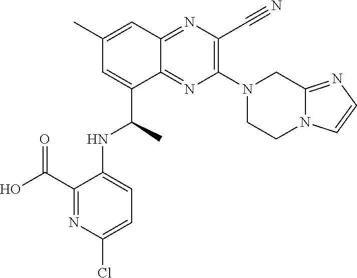 [SMILES: Cc1cc([C@@H](C)Nc2ccc(Cl)nc2C(=O)O)c2nc(N3CCn4ccnc4C3)c(C#N)nc2c1] |
| 583 | (R)-6-chloro-3-((1-(2-cyano-3-(2,3- dimethyl-5,6-dihydroimidazo[1,2- a]pyrazin-7(8H)-yl)-7- methylquinoxalin-5- yl)ethyl)amino)picolinic acid | 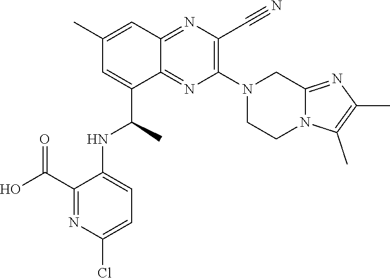 [SMILES: Cc1cc([C@@H](C)Nc2ccc(Cl)nc2C(=O)O)c2nc(N3CCn4c(nc(C)c4C)C3)c(C#N)nc2c1] |
| 584 | (R)-6-chloro-3-((1-(2-cyano-7-methyl- 3-(2-(trifluoromethyl)-5,6- dihydroimidazo[1,2-a]pyrazin-7(8H)- yl)quinoxalin-5- yl)ethyl)amino)picolinic acid | 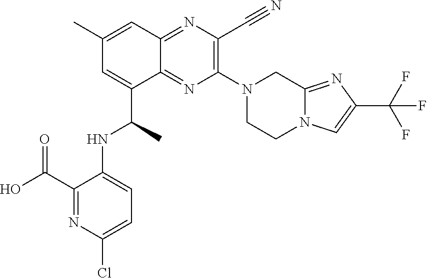 [SMILES: Cc1cc([C@@H](C)Nc2ccc(Cl)nc2C(=O)O)c2nc(N3CCn4cc(C(F)(F)F)nc4C3)c(C#N)nc2c1] |
| 585 | (R)-6-chloro-3-((1-(2-cyano-7-methyl- 3-(4H-thieno[3,4-c]pyrrol-5(6H)- yl)quinoxalin-5- yl)ethyl)amino)picolinic acid | 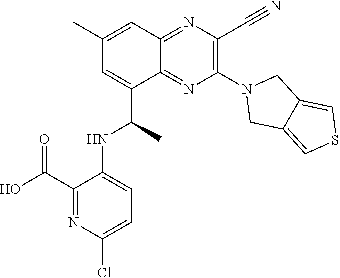 [SMILES: Cc1cc([C@@H](C)Nc2ccc(Cl)nc2C(=O)O)c2nc(N3Cc4cscc4C3)c(C#N)nc2c1] |
| 586 | (R)-6-chloro-3-((1-(2-cyano-3-(5,6- dihydroimidazo[1,5-a]pyrazin-7(8H)- yl)-7-methylquinoxalin-5- yl)ethyl)amino)picolinic acid | 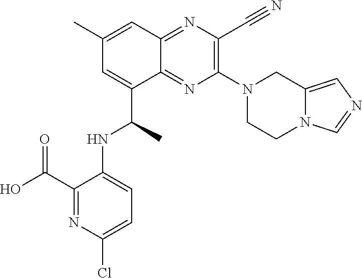 [SMILES: Cc1cc([C@@H](C)Nc2ccc(Cl)nc2C(=O)O)c2nc(N3CCn4cncc4C3)c(C#N)nc2c1] |
| 587 | (R)-6-chloro-3-((1-(2-cyano-3-(6,7- dihydropyrazolo[1,5-a]pyrazin-5(4H)- yl)-7-methylquinoxalin-5- yl)ethyl)amino)picolinic acid | 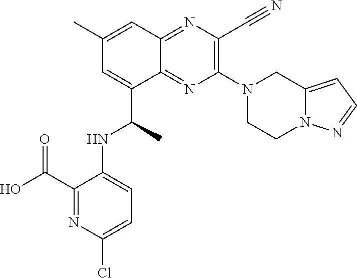 [SMILES: Cc1cc([C@@H](C)Nc2ccc(Cl)nc2C(=O)O)c2nc(N3CCn4nccc4C3)c(C#N)nc2c1] |
| 588 | (R)-6-chloro-3-((1-(2-cyano-7-methyl- 3-(piperazin-1-yl)quinoxalin-5- yl)ethyl)amino)picolinic acid | 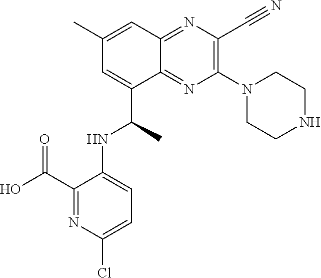 [SMILES: Cc1cc([C@@H](C)Nc2ccc(Cl)nc2C(=O)O)c2nc(N3CCNCC3)c(C#N)nc2c1] |
| 589 | (R)-6-chloro-3-((1-(2-cyano-3-(4-(2,2- difluoroethyl)piperazin-1-yl)-7- methylquinoxalin-5- yl)ethyl)amino)picolinic acid | 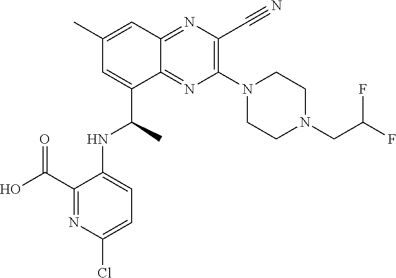 [SMILES: Cc1cc([C@@H](C)Nc2ccc(Cl)nc2C(=O)O)c2nc(N3CCN(CC(F)F)CC3)c(C#N)nc2c1] |
| 590 | (R)-6-chloro-3-((1-(2-cyano-7-methyl- 3-(4-(2,2,2-trifluoroethyl)piperazin-1- yl)quinoxalin-5- yl)ethyl)amino)picolinic acid | 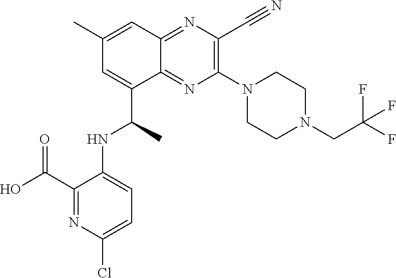 [SMILES: Cc1cc([C@@H](C)Nc2ccc(Cl)nc2C(=O)O)c2nc(N3CCN(CC(F)(F)F)CC3)c(C#N)nc2c1] |
| 591 | (R)-6-chloro-3-((1-(2-cyano-7-methyl- 3-(4-methylpiperazin-1-yl)quinoxalin- 5-yl)ethyl)amino)picolinic acid | 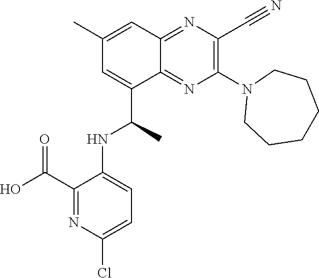 [SMILES: Cc1cc([C@@H](C)Nc2ccc(Cl)nc2C(=O)O)c2nc(N3CCCCCC3)c(C#N)nc2c1] |
| 592 | (R)-3-((1-(3-(azepan-1-yl)-2-cyano-7- methylquinoxalin-5-yl)ethyl)amino)-6- chloropicolinic acid | 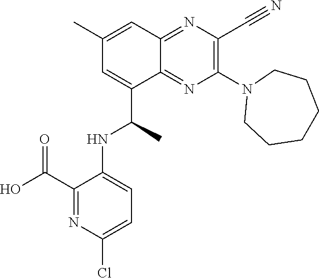 [SMILES: Cc1cc([C@@H](C)Nc2ccc(Cl)nc2C(=O)O)c2nc(N3CCCCCC3)c(C#N)nc2c1] |
| 593 | (R)-6-chloro-3-((1-(2-cyano-7-methyl- 3-(piperidin-1-yl)quinoxalin-5- yl)ethyl)amino)picolinic acid | 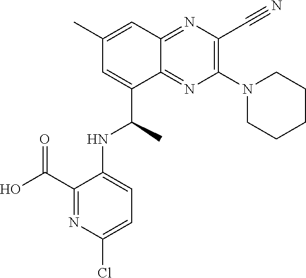 [SMILES: Cc1cc([C@@H](C)Nc2ccc(Cl)nc2C(=O)O)c2nc(N3CCCCC3)c(C#N)nc2c1] |
| 594 | (R)-6-chloro-3-((1-(2-cyano-7-methyl- 3-(2-oxa-8-azaspiro[4.5]decan-8- yl)quinoxalin-5- yl)ethyl)amino)picolinic acid | 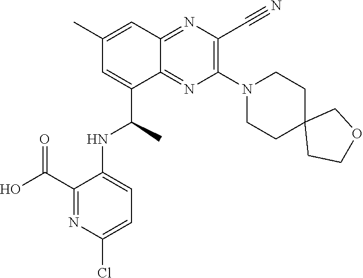 [SMILES: Cc1cc([C@@H](C)Nc2ccc(Cl)nc2C(=O)O)c2nc(N3CCC4(CCOC4)CC3)c(C#N)nc2c1] |
| 595 | (R)-6-chloro-3-((1-(2-cyano-7-methyl- 3-(6-azaspiro[2.5]octan-6- yl)quinoxalin-5- yl)ethyl)amino)picolinic acid | 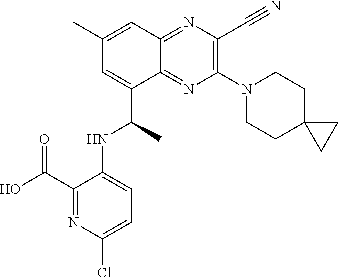 [SMILES: Cc1cc([C@@H](C)Nc2ccc(Cl)nc2C(=O)O)c2nc(N3CCC4(CC3)CC4)c(C#N)nc2c1] |
| 596 | (R)-3-((1-(3-(azetidin-1-yl)-2-cyano-7- methylquinoxalin-5-yl)ethyl)amino)-6- chloropicolinic acid | 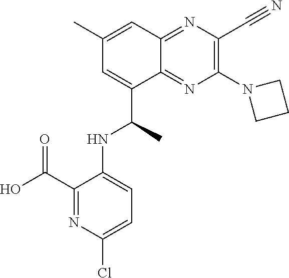 [SMILES: Cc1cc([C@@H](C)Nc2ccc(Cl)nc2C(=O)O)c2nc(N3CCC3)c(C#N)nc2c1] |
| 597 | 6-chloro-3-(((1R)-1-(2-cyano-3-(2- hydroxy-8-azaspiro[4.5]decan-8-yl)-7- methylquinoxalin-5- yl)ethyl)amino)picolinic acid | 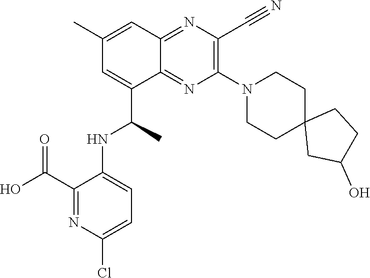 [SMILES: Cc1cc([C@@H](C)Nc2ccc(Cl)nc2C(=O)O)c2nc(N3CCC4(CCC(O)C4)CC3)c(C#N)nc2c1] |
| 598 | (R)-6-chloro-3-((1-(2-cyano-3-(2,2- difluoro-7-azaspiro[3.5]nonan-7-yl)-7- methylquinoxalin-5- yl)ethyl)amino)picolinic acid | 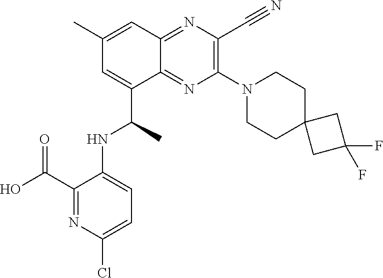 [SMILES: Cc1cc([C@@H](C)Nc2ccc(Cl)nc2C(=O)O)c2nc(N3CCC4(CC3)CC(F)(F)C4)c(C#N)nc2c1] |
| 599 | (R)-6-chloro-3-((1-(2-cyano-7-methyl- 3-(4-methylpiperidin-1-yl)quinoxalin- 5-yl)ethyl)amino)picolinic acid | 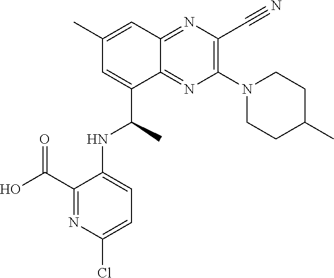 [SMILES: Cc1cc([C@@H](C)Nc2ccc(Cl)nc2C(=O)O)c2nc(N3CCC(C)CC3)c(C#N)nc2c1] |
| 600 | 6-chloro-3-(((1R)-1-(2-cyano-7- methyl-3-(octahydro-2H-4,7- ethanoisoindol-2-yl)quinoxalin-5- yl)ethyl)amino)picolinic acid | 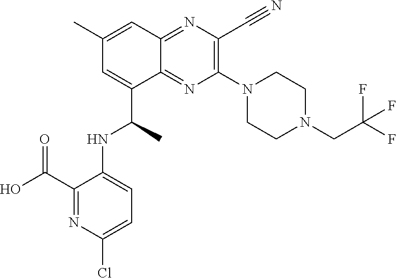 [SMILES: Cc1cc([C@@H](C)Nc2ccc(Cl)nc2C(=O)O)c2nc(N3CCN(CC(F)(F)F)CC3)c(C#N)nc2c1] |
| 601 | 6-chloro-3-(((1R)-1-(2-cyano-3-(6,6- dimethyl-3-azabicyclo[3.2.0]heptan-3- yl)-7-methylquinoxalin-5- yl)ethyl)amino)picolinic acid | 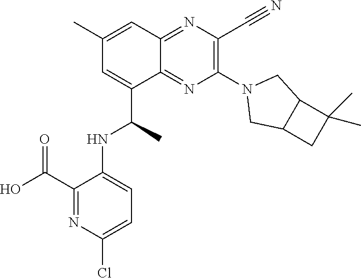 [SMILES: Cc1cc([C@@H](C)Nc2ccc(Cl)nc2C(=O)O)c2nc(N3CC4CC(C)(C)C4C3)c(C#N)nc2c1] |
| 602 | (R)-6-chloro-3-((1-(2-cyano-7-methyl- 3-(2-azaspiro[3.3]heptan-2- yl)quinoxalin-5- yl)ethyl)amino)picolinic acid | 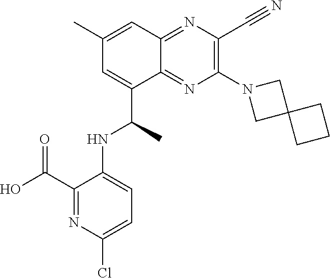 [SMILES: Cc1cc([C@@H](C)Nc2ccc(Cl)nc2C(=O)O)c2nc(N3CC4(CCC4)C3)c(C#N)nc2c1] |
| 603 | (R)-6-chloro-3-((1-(2-cyano-3-(2- azadispiro[3.1.36.14]decan-2-yl)-7- methylquinoxalin-5- yl)ethyl)amino)picolinic acid | 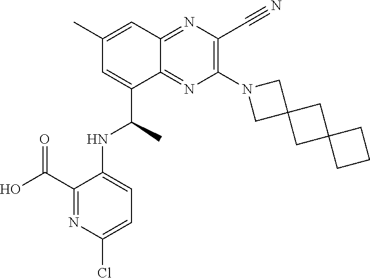 [SMILES: Cc1cc([C@@H](C)Nc2ccc(Cl)nc2C(=O)O)c2nc(N3CC4(C3)CC3(CCC3)C4)c(C#N)nc2c1] |
| 604 | 6-chloro-3-(((1R)-1-(2-cyano-7- methyl-3-(5- methylhexahydropyrrolo[3,4-c]pyrrol- 2(1H)-yl)quinoxalin-5- yl)ethyl)amino)picolinic acid | 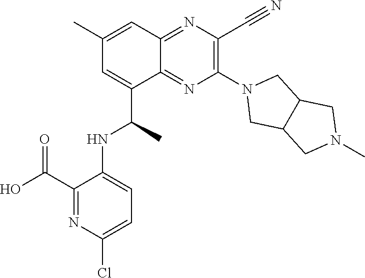 [SMILES: Cc1cc([C@@H](C)Nc2ccc(Cl)nc2C(=O)O)c2nc(N3CC4CN(C)CC4C3)c(C#N)nc2c1] |
| 605 | 6-chloro-3-(((1R)-1-(2-cyano-3-(2- (fluoromethyl)morpholino)-7- methylquinoxalin-5- yl)ethyl)amino)picolinic acid | 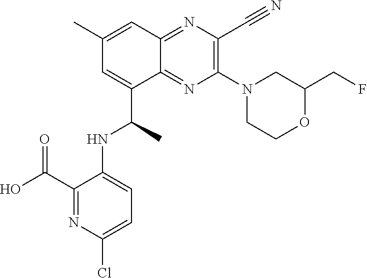 [SMILES: Cc1cc([C@@H](C)Nc2ccc(Cl)nc2C(=O)O)c2nc(N3CCOC(CF)C3)c(C#N)nc2c1] |
| 606 | 6-chloro-3-(((1R)-1-(2-cyano-3-(2-(2- fluoroethyl)morpholino)-7- methylquinoxalin-5- yl)ethyl)amino)picolinic acid | 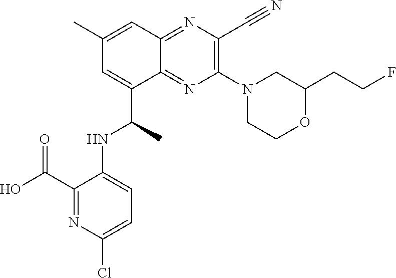 [SMILES: Cc1cc([C@@H](C)Nc2ccc(Cl)nc2C(=O)O)c2nc(N3CCOC(CCF)C3)c(C#N)nc2c1] |
| 607 | 6-chloro-3-(((1R)-1-(2-cyano-3-(5,5- difluorohexahydrocyclopenta[c]pyrrol- 2(1H)-yl)-7-methylquinoxalin-5- yl)ethyl)amino)picolinic acid | 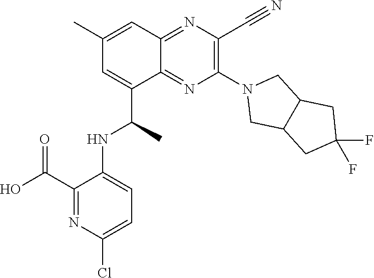 [SMILES: Cc1cc([C@@H](C)Nc2ccc(Cl)nc2C(=O)O)c2nc(N3CC4CC(F)(F)CC4C3)c(C#N)nc2c1] |
| 608 | 6-chloro-3-(((1R)-1-(2-cyano-3-(2- cyclopropylmorpholino)-7- methylquinoxalin-5- yl)ethyl)amino)picolinic acid | 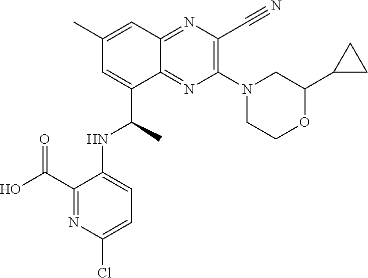 [SMILES: Cc1cc([C@@H](C)Nc2ccc(Cl)nc2C(=O)O)c2nc(N3CCOC(C4CC4)C3)c(C#N)nc2c1] |
| 609 | 6-chloro-3-(((1R)-1-(2-cyano-3-(2- cyclopropyl-6-methylmorpholino)-7- methylquinoxalin-5- yl)ethyl)amino)picolinic acid | 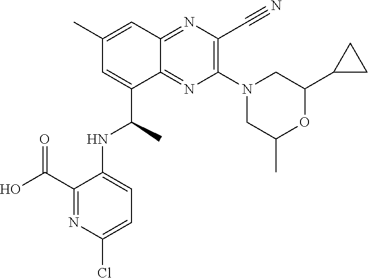 [SMILES: Cc1cc([C@@H](C)Nc2ccc(Cl)nc2C(=O)O)c2nc(N3CC(C)OC(C4CC4)C3)c(C#N)nc2c1] |
| 610 | (R)-3-((1-(3-(3- azabicyclo[3.1.1]heptan-3-yl)-2- cyano-7-methylquinoxalin-5- yl)ethyl)amino)-6-chloropicolinic acid | 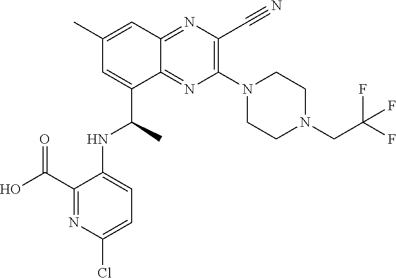 [SMILES: Cc1cc([C@@H](C)Nc2ccc(Cl)nc2C(=O)O)c2nc(N3CCN(CC(F)(F)F)CC3)c(C#N)nc2c1] |
| 611 | 3-(((1R)-1-(3-(3- azabicyclo[3.1.0]hexan-3-yl)-2-cyano- 7-methylquinoxalin-5-yl)ethyl)amino)- 6-chloropicolinic acid | 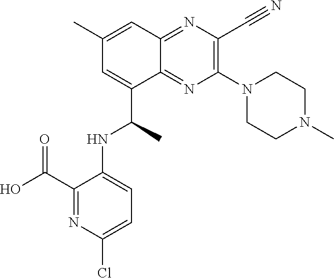 [SMILES: Cc1cc([C@@H](C)Nc2ccc(Cl)nc2C(=O)O)c2nc(N3CCN(C)CC3)c(C#N)nc2c1] |
| 612 | (R)-6-chloro-3-((1-(2-cyano-3-(6-(4- cyanophenyl)-2,6- diazaspiro[3.3]heptan-2-yl)-7- methylquinoxalin-5- yl)ethyl)amino)picolinic acid | 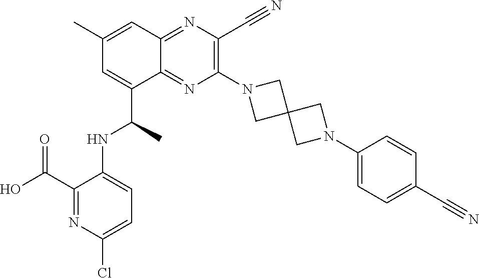 [SMILES: Cc1cc([C@@H](C)Nc2ccc(Cl)nc2C(=O)O)c2nc(N3CC4(CN(c5ccc(C#N)cc5)C4)C3)c(C#N)nc2c1] |
| 613 | (R)-6-chloro-3-((1-(2-cyano-3-(2,2- difluoro-6-azaspiro[3.4]octan-6-yl)-7- methylquinoxalin-5- yl)ethyl)amino)picolinic acid | 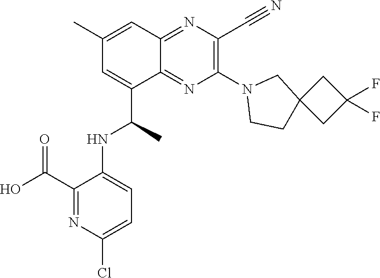 [SMILES: Cc1cc([C@@H](C)Nc2ccc(Cl)nc2C(=O)O)c2nc(N3CCC4(C3)CC(F)(F)C4)c(C#N)nc2c1] |
| 614 | (R)-6-chloro-3-((1-(2-cyano-3-(6,6- difluoro-2-azaspiro[3.3]heptan-2-yl)- 7-methylquinoxalin-5- yl)ethyl)amino)picolinic acid | 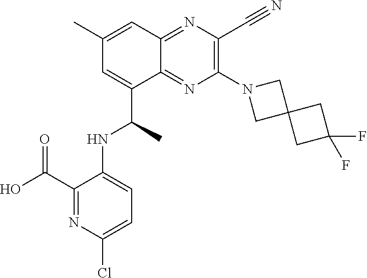 [SMILES: Cc1cc([C@@H](C)Nc2ccc(Cl)nc2C(=O)O)c2nc(N3CC4(C3)CC(F)(F)C4)c(C#N)nc2c1] |
| 615 | 6-chloro-3-(((1R)-1-(2-cyano-3-(2- cyclopropyl-2-methylmorpholino)-7- methylquinoxalin-5- yl)ethyl)amino)picolinic acid | 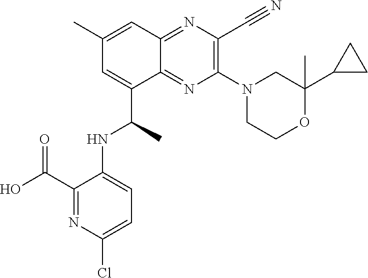 [SMILES: Cc1cc([C@@H](C)Nc2ccc(Cl)nc2C(=O)O)c2nc(N3CCOC(C)(C4CC4)C3)c(C#N)nc2c1] |
| 616 | 3-(((1R)-1-(3-(3,6- diazabicyclo[3.1.1]heptan-6-yl)-2- cyano-7-methylquinoxalin-5- yl)ethyl)amino)-6-chloropicolinic acid | 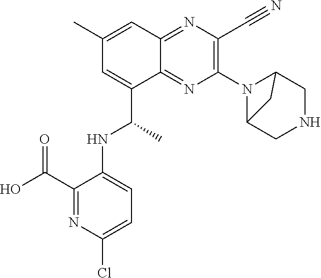 [SMILES: Cc1cc([C@H](C)Nc2ccc(Cl)nc2C(=O)O)c2nc(N3C4CNCC3C4)c(C#N)nc2c1] |
| 617 | (R)-3-((1-(3-(7- azabicyclo[2.2.1]heptan-7-yl)-2- cyano-7-methylquinoxalin-5- yl)ethyl)amino)-6-chloropicolinic acid | 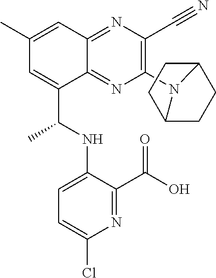 [SMILES: Cc1cc([C@@H](C)Nc2ccc(Cl)nc2C(=O)O)c2nc(N3C4CCC3CC4)c(C#N)nc2c1] |
| 618 | (R)-3-((1-(3-(2-azaadamantan-2-yl)- 2-cyano-7-methylquinoxalin-5- yl)ethyl)amino)-6-chloropicolinic acid | 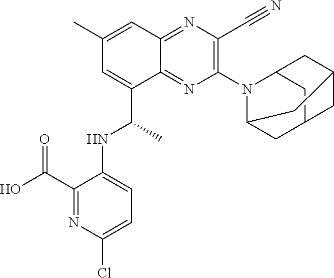 [SMILES: Cc1cc([C@H](C)Nc2ccc(Cl)nc2C(=O)O)c2nc(N3C4CC5CC(C4)CC3C5)c(C#N)nc2c1] |
| 619 | (R)-6-chloro-3-((1-(2-cyano-3-(1- (methoxymethyl)-4-methyl-2- azabicyclo[2.1.1]hexan-2-yl)-7- methylquinoxalin-5- yl)ethyl)amino)picolinic acid | 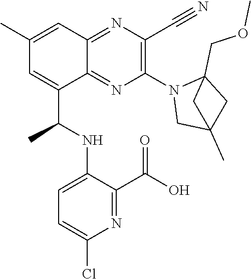 |
| 620 | 6-chloro-3-(((1R)-1-(2-cyano-3-(8- ((dimethylamino)methyl)-6-oxa-9- azaspiro[4.5]decan-9-yl)-7- methylquinoxalin-5- yl)ethyl)amino)picolinic acid | 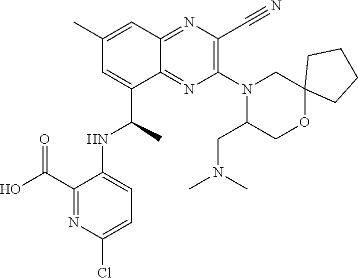 [SMILES: Cc1cc([C@@H](C)Nc2ccc(Cl)nc2C(=O)O)c2nc(N3CC4(CCCC4)OCC3CN(C)C)c(C#N)nc2c1] |
| 621 | 6-chloro-3-(((1R)-1-(2-cyano-3-(2- (fluoromethyl)azetidin-1-yl)-7- methylquinoxalin-5- yl)ethyl)amino)picolinic acid | 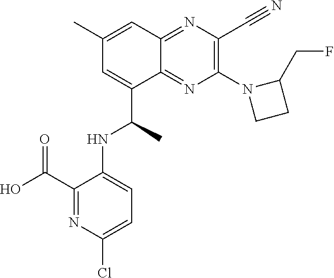 [SMILES: Cc1cc([C@@H](C)Nc2ccc(Cl)nc2C(=O)O)c2nc(N3CCC3CF)c(C#N)nc2c1] |
| 622 | (R)-3-((1-(3-(2- azabicyclo[2.2.2]octan-2-yl)-2-cyano- 7-methylquinoxalin-5-yl)ethyl)amino)- 6-chloropicolinic acid | 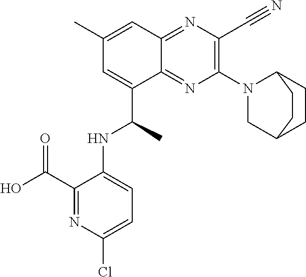 [SMILES: Cc1cc([C@@H](C)Nc2ccc(Cl)nc2C(=O)O)c2nc(N3CC4CCC3CC4)c(C#N)nc2c1] |
| 623 | 3-(((1R)-1-(3-(2- azabicyclo[2.2.1]heptan-2-yl)-2- cyano-7-methylquinoxalin-5- yl)ethyl)amino)-6-chloropicolinic acid | 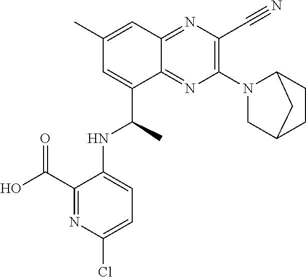 [SMILES: Cc1cc([C@@H](C)Nc2ccc(Cl)nc2C(=O)O)c2nc(N3CC4CCC3C4)c(C#N)nc2c1] |
| 624 | 6-chloro-3-(((1R)-1-(2-cyano-3- (hexahydro-3,5- methanocyclopenta[b]pyrrol-1(2H)- yl)-7-methylquinoxalin-5- yl)ethyl)amino)picolinic acid | 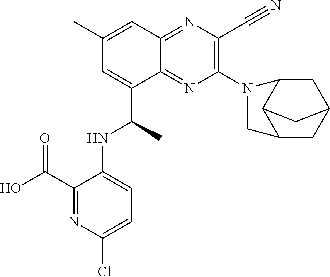 [SMILES: Cc1cc([C@@H](C)Nc2ccc(Cl)nc2C(=O)O)c2nc(N3CC4CC5CC4C3C5)c(C#N)nc2c1] |
| 625 | 6-chloro-3-(((1R)-1-(2-cyano-3-(5- (2,2-difluoroethyl)-2,5- diazabicyclo[2.2.1]heptan-2-yl)-7- methylquinoxalin-5- yl)ethyl)amino)picolinic acid | 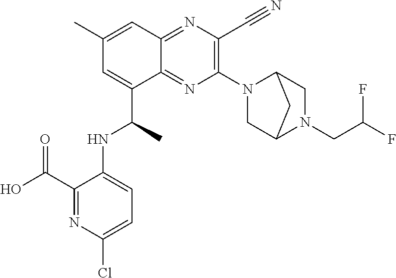 [SMILES: Cc1cc([C@@H](C)Nc2ccc(Cl)nc2C(=O)O)c2nc(N3CC4CC3CN4CC(F)F)c(C#N)nc2c1] |
| 626 | 6-chloro-3-(((1R)-1-(2-cyano-3-(1- (hydroxymethyl)-6- azabicyclo[3.2.1]octan-6-yl)-7- methylquinoxalin-5- yl)ethyl)amino)picolinic acid | 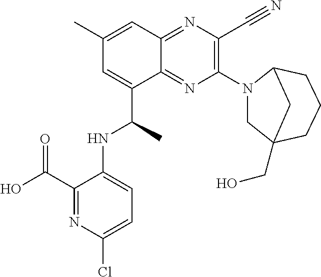 [SMILES: Cc1cc([C@@H](C)Nc2ccc(Cl)nc2C(=O)O)c2nc(N3CC4(CO)CCCC3C4)c(C#N)nc2c1] |
| 627 | (R)-3-((1-(3-(2- azabicyclo[2.1.1]hexan-2-yl)-2-cyano- 7-methylquinoxalin-5-yl)ethyl)amino)- 6-chloropicolinic acid | 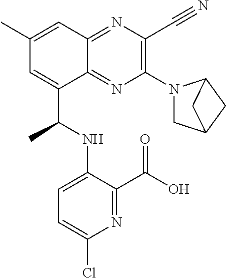 [SMILES: Cc1cc([C@H](C)Nc2ccc(Cl)nc2C(=O)O)c2nc(N3CC4CC3C4)c(C#N)nc2c1] |
| 628 | 6-chloro-3-(((1R)-1-(2-cyano-7- methyl-3-(2-methyl-4-(pyridin-3- ylmethyl)piperazin-1-yl)quinoxalin-5- yl)ethyl)amino)picolinic acid | 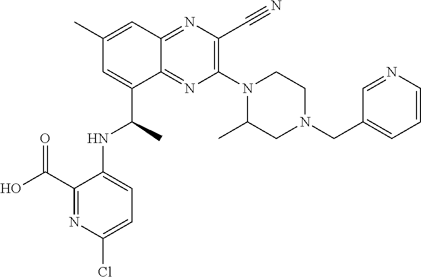 [SMILES: Cc1cc([C@@H](C)Nc2ccc(Cl)nc2C(=O)O)c2nc(N3CCN(Cc4cccnc4)CC3C)c(C#N)nc2c1] |
| 629 | 6-chloro-3-(((1R)-1-(2-cyano-7- methyl-3-(2-methylazetidin-1- yl)quinoxalin-5- yl)ethyl)amino)picolinic acid | 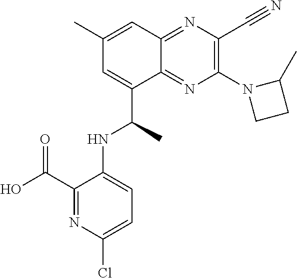 [SMILES: Cc1cc([C@@H](C)Nc2ccc(Cl)nc2C(=O)O)c2nc(N3CCC3C)c(C#N)nc2c1] |
| 630 | 6-chloro-3-(((1R)-1-(2-cyano-3-(2,3- dimethylmorpholino)-7- methylquinoxalin-5- yl)ethyl)amino)picolinic acid | 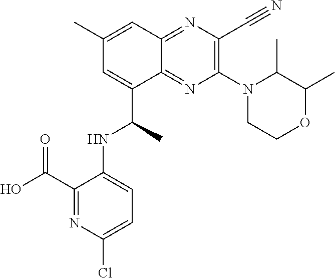 [SMILES: Cc1cc([C@@H](C)Nc2ccc(Cl)nc2C(=O)O)c2nc(N3CCOC(C)C3C)c(C#N)nc2c1] |
| 631 | (R)-6-chloro-3-((1-(2-cyano-7-methyl- 3-(1-oxoisoindolin-2-yl)quinoxalin-5- yl)ethyl)amino)picolinic acid | 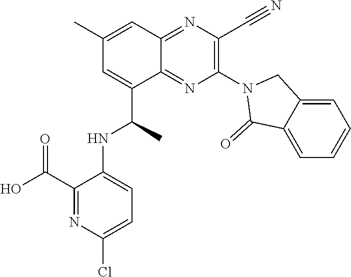 [SMILES: Cc1cc([C@@H](C)Nc2ccc(Cl)nc2C(=O)O)c2nc(N3Cc4ccccc4C3=O)c(C#N)nc2c1] |
| 632 | (R)-6-chloro-3-((1-(2-cyano-7-methyl- 3-(4-methyl-3-oxopiperazin-1- yl)quinoxalin-5- yl)ethyl)amino)picolinic acid | 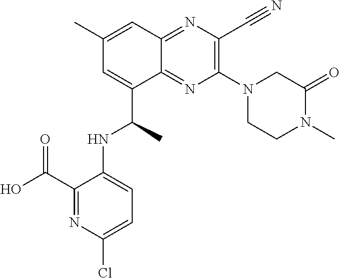 [SMILES: Cc1cc([C@@H](C)Nc2ccc(Cl)nc2C(=O)O)c2nc(N3CCN(C)C(=O)C3)c(C#N)nc2c1] |
| 633 | (R)-6-chloro-3-((1-(2-cyano-7-methyl- 3-(3-oxopiperazin-1-yl)quinoxalin-5- yl)ethyl)amino)picolinic acid | 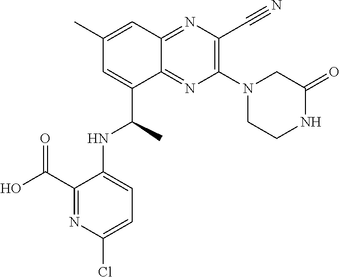 [SMILES: Cc1cc([C@@H](C)Nc2ccc(Cl)nc2C(=O)O)c2nc(N3CCNC(=O)C3)c(C#N)nc2c1] |
| 634 | 6-chloro-3-(((1R)-1-(2-cyano-3-(3,3- difluoro-4-methoxypiperidin-1-yl)-7- methylquinoxalin-5- yl)ethyl)amino)picolinic acid | 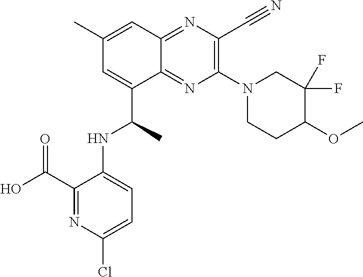 [SMILES: COC1CCN(c2nc3c([C@@H](C)Nc4ccc(Cl)nc4C(=O)O)cc(C)cc3nc2C#N)CC1(F)F] |
| 635 | 6-chloro-3-(((1R)-1-(2-cyano-3-(3- hydroxy-2-(2- methoxyphenyl)pyrrolidin-1-yl)-7- methylquinoxalin-5- yl)ethyl)amino)picolinic acid | 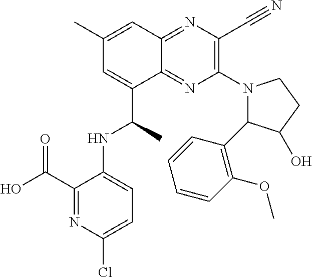 [SMILES: COc1ccccc1C1C(O)CCN1c1nc2c([C@@H](C)Nc3ccc(Cl)nc3C(=O)O)cc(C)cc2nc1C#N] |
| 636 | 6-chloro-3-(((1R)-1-(2-cyano-3-(3,3- difluoro-4-methylpiperidin-1-yl)-7- methylquinoxalin-5- yl)ethyl)amino)picolinic acid | 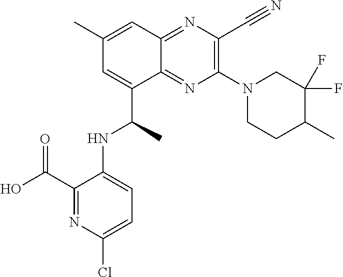 [SMILES: Cc1cc([C@@H](C)Nc2ccc(Cl)nc2C(=O)O)c2nc(N3CCC(C)C(F)(F)C3)c(C#N)nc2c1] |
| 637 | (R)-6-chloro-3-((1-(2-cyano-3-(3,4- dihydroquinolin-1(2H)-yl)-7- methylquinoxalin-5- yl)ethyl)amino)picolinic acid | 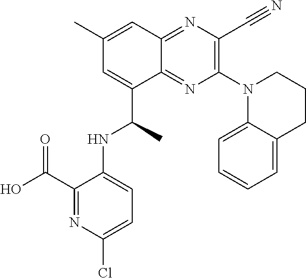 [SMILES: Cc1cc([C@@H](C)Nc2ccc(Cl)nc2C(=O)O)c2nc(N3CCCc4ccccc43)c(C#N)nc2c1] |
| 638 | 6-chloro-3-(((1R)-1-(2-cyano-3-(3- hydroxypiperidin-1-yl)-7- methylquinoxalin-5- yl)ethyl)amino)picolinic acid | 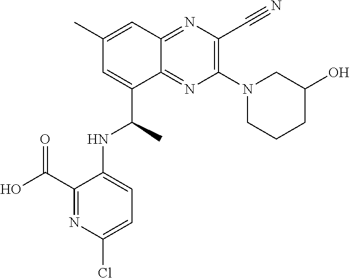 [SMILES: Cc1cc([C@@H](C)Nc2ccc(Cl)nc2C(=O)O)c2nc(N3CCCC(O)C3)c(C#N)nc2c1] |
| 639 | 6-chloro-3-(((1R)-1-(2-cyano-3-(3- hydroxy-3-methylpiperidin-1-yl)-7- methylquinoxalin-5- yl)ethyl)amino)picolinic acid | 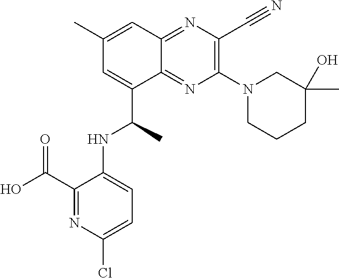 [SMILES: Cc1cc([C@@H](C)Nc2ccc(Cl)nc2C(=O)O)c2nc(N3CCCC(C)(O)C3)c(C#N)nc2c1] |
| 640 | 6-chloro-3-(((1R)-1-(2-cyano-3-(2-(4- hydroxy-4-methyltetrahydro-2H- pyran-3-yl)pyrrolidin-1-yl)-7- methylquinoxalin-5- yl)ethyl)amino)picolinic acid | 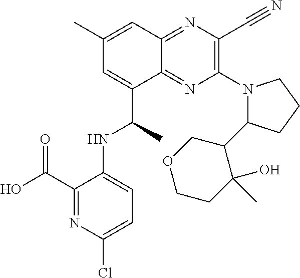 [SMILES: Cc1cc([C@@H](C)Nc2ccc(Cl)nc2C(=O)O)c2nc(N3CCCC3C3COCCC3(C)O)c(C#N)nc2c1] |
| 641 | 6-chloro-3-(((1R)-1-(2-cyano-3-(4,4- difluoro-3-methoxypiperidin-1-yl)-7- methylquinoxalin-5- yl)ethyl)amino)picolinic acid | 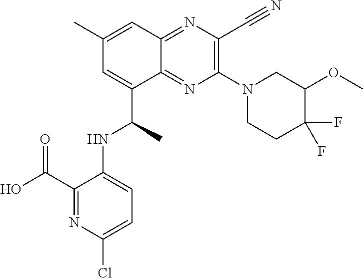 [SMILES: COC1CN(c2nc3c([C@@H](C)Nc4ccc(Cl)nc4C(=O)O)cc(C)cc3nc2C#N)CCC1(F)F] |
| 642 | 6-chloro-3-(((1R)-1-(2-cyano-7- methyl-3-(3,4,4-trifluoropiperidin-1- yl)quinoxalin-5- yl)ethyl)amino)picolinic acid | 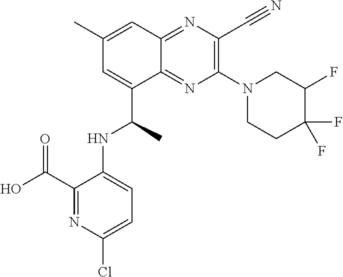 [SMILES: Cc1cc([C@@H](C)Nc2ccc(Cl)nc2C(=O)O)c2nc(N3CCC(F)(F)C(F)C3)c(C#N)nc2c1] |
| 643 | 6-chloro-3-(((1R)-1-(2-cyano-3-(9,9- difluoro-3-oxa-7- azabicyclo[3.3.1]nonan-7-yl)-7- methylquinoxalin-5- yl)ethyl)amino)picolinic acid | 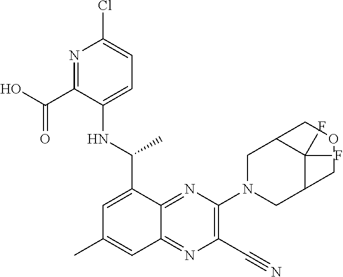 [SMILES: Cc1cc([C@@H](C)Nc2ccc(Cl)nc2C(=O)O)c2nc(N3CC4COCC(C3)C4(F)F)c(C#N)nc2c1] |
| 644 | 6-chloro-3-(((1R)-1-(2-cyano-3-(9,9- difluoro-3-azabicyclo[3.3.1]nonan-3- yl)-7-methylquinoxalin-5- yl)ethyl)amino)picolinic acid | 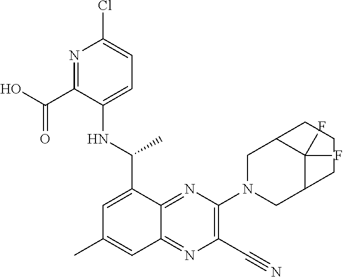 [SMILES: Cc1cc([C@@H](C)Nc2ccc(Cl)nc2C(=O)O)c2nc(N3CC4CCCC(C3)C4(F)F)c(C#N)nc2c1] |
| 645 | 6-chloro-3-(((1R)-1-(2-cyano-3-(9,9- difluoro-1-(hydroxymethyl)-3- azabicyclo[3.3.1]nonan-3-yl)-7- methylquinoxalin-5- yl)ethyl)amino)picolinic acid | 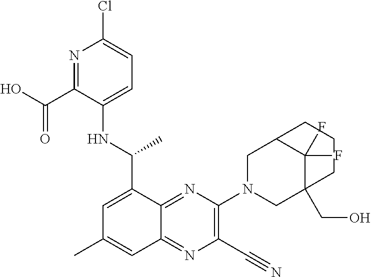 [SMILES: Cc1cc([C@@H](C)Nc2ccc(Cl)nc2C(=O)O)c2nc(N3CC4CCCC(CO)(C3)C4(F)F)c(C#N)nc2c1] |
| 646 | 6-chloro-3-(((1R)-1-(2-cyano-3-(8- hydroxy-3-azabicyclo[3.2.1]octan-3- yl)-7-methylquinoxalin-5- yl)ethyl)amino)picolinic acid | 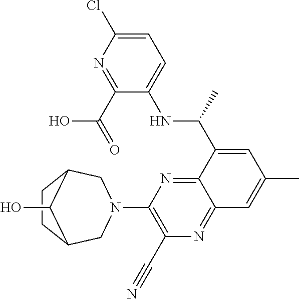 [SMILES: Cc1cc([C@@H](C)Nc2ccc(Cl)nc2C(=O)O)c2nc(N3CC4CCC(C3)C4O)c(C#N)nc2c1] |
| 647 | 6-chloro-3-(((1R)-1-(2-cyano-3-(8,8- difluoro-3-azabicyclo[3.2.1]octan-3- yl)-7-methylquinoxalin-5- yl)ethyl)amino)picolinic acid | 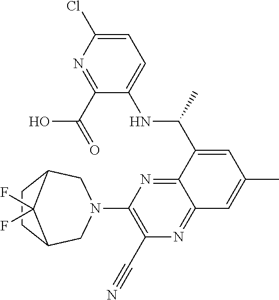 [SMILES: Cc1cc([C@@H](C)Nc2ccc(Cl)nc2C(=O)O)c2nc(N3CC4CCC(C3)C4(F)F)c(C#N)nc2c1] |
| 648 | 6-chloro-3-(((1R)-1-(2-cyano-3-(5- hydroxy-2-azabicyclo[2.2.2]octan-2- yl)-7-methylquinoxalin-5- yl)ethyl)amino)picolinic acid | 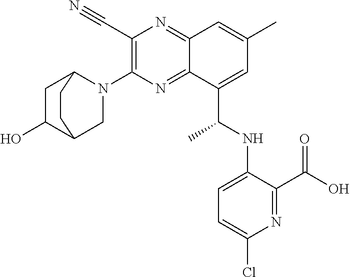 [SMILES: Cc1cc([C@@H](C)Nc2ccc(Cl)nc2C(=O)O)c2nc(N3CC4CCC3CC4O)c(C#N)nc2c1] |
| 649 | 6-chloro-3-(((1R)-1-(2-cyano-3-(5,5- difluoro-2-azabicyclo[2.2.2]octan-2- yl)-7-methylquinoxalin-5- yl)ethyl)amino)picolinic acid | 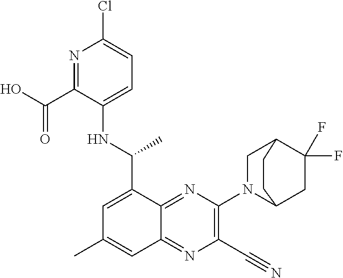 [SMILES: Cc1cc([C@@H](C)Nc2ccc(Cl)nc2C(=O)O)c2nc(N3CC4CCC3CC4(F)F)c(C#N)nc2c1] |
| 650 | 6-chloro-3-(((1R)-1-(2-cyano-7- methyl-3-(7,7,9,9-tetrafluoro-3- azabicyclo[3.3.1]nonan-3- yl)quinoxalin-5- yl)ethyl)amino)picolinic acid | 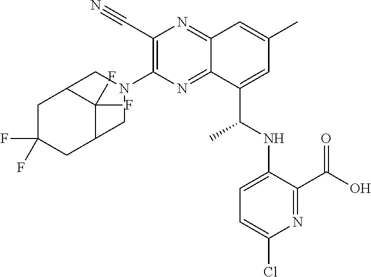 [SMILES: Cc1cc([C@@H](C)Nc2ccc(Cl)nc2C(=O)O)c2nc(N3CC4CC(F)(F)CC(C3)C4(F)F)c(C#N)nc2c1] |
| 651 | 6-chloro-3-(((1R)-1-(2-cyano-3-(6- hydroxyhexahydro-3,5- methanocyclopenta[b]pyrrol-1(2H)- yl)-7-methylquinoxalin-5- yl)ethyl)amino)picolinic acid | 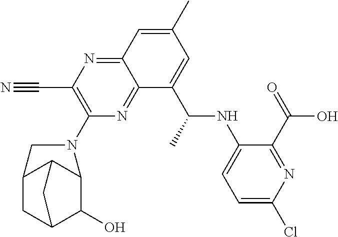 [SMILES: Cc1cc([C@@H](C)Nc2ccc(Cl)nc2C(=O)O)c2nc(N3CC4CC5CC4C3C5O)c(C#N)nc2c1] |
| 652 | 6-chloro-3-(((1R)-1-(2-cyano-3-(6-(4- cyanophenyl)-3,6- diazabicyclo[3.1.1]heptan-3-yl)-7- methylquinoxalin-5- yl)ethyl)amino)picolinic acid | 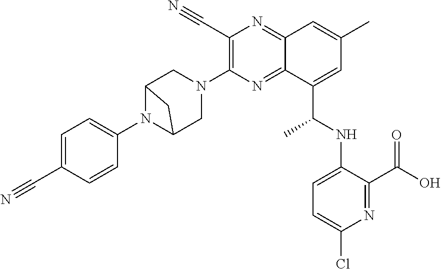 [SMILES: Cc1cc([C@@H](C)Nc2ccc(Cl)nc2C(=O)O)c2nc(N3CC4CC(C3)N4c3ccc(C#N)cc3)c(C#N)nc2c1] |
| 653 | 6-chloro-3-(((1R)-1-(2-cyano-3-(6- hydroxy-6-methyl-3- azabicyclo[3.1.1]heptan-3-yl)-7- methylquinoxalin-5- yl)ethyl)amino)picolinic acid | 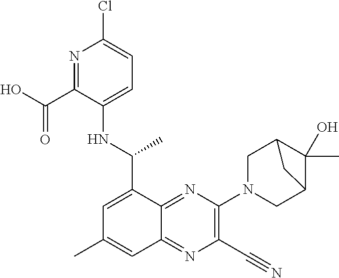 [SMILES: Cc1cc([C@@H](C)Nc2ccc(Cl)nc2C(=O)O)c2nc(N3CC4CC(C3)C4(C)O)c(C#N)nc2c1] |
| 654 | 6-chloro-3-(((1R)-1-(2-cyano-3-(5-(4- cyanophenyl)-2,5- diazabicyclo[2.2.1]heptan-2-yl)-7- methylquinoxalin-5- yl)ethyl)amino)picolinic acid | 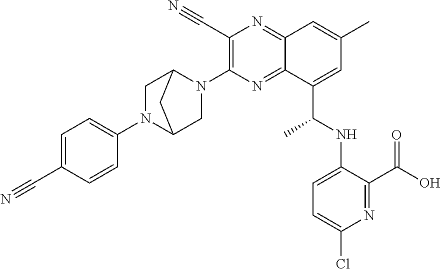 [SMILES: Cc1cc([C@@H](C)Nc2ccc(Cl)nc2C(=O)O)c2nc(N3CC4CC3CN4c3ccc(C#N)cc3)c(C#N)nc2c1] |
| 655 | 6-chloro-3-(((1R)-1-(2-cyano-7- methyl-3-(5-phenyl-2- azabicyclo[2.2.1]heptan-2- yl)quinoxalin-5- yl)ethyl)amino)picolinic acid | 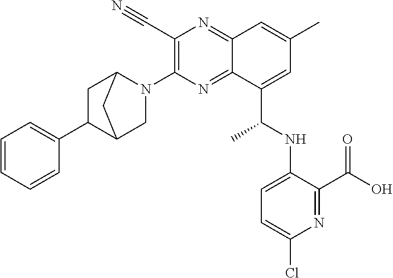 [SMILES: Cc1cc([C@@H](C)Nc2ccc(Cl)nc2C(=O)O)c2nc(N3CC4CC3CC4c3ccccc3)c(C#N)nc2c1] |
| 656 | 6-chloro-3-(((1R)-1-(2-cyano-3-(4,4- difluoro-3,5-dimethylpiperidin-1-yl)-7- methylquinoxalin-5- yl)ethyl)amino)picolinic acid | 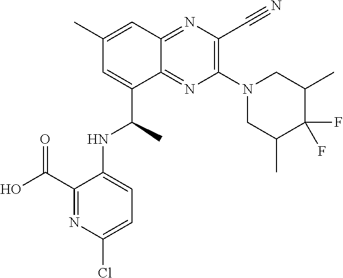 [SMILES: Cc1cc([C@@H](C)Nc2ccc(Cl)nc2C(=O)O)c2nc(N3CC(C)C(F)(F)C(C)C3)c(C#N)nc2c1] |
| 657 | 3-(((1R)-1-(3-(7-(tert-butyl)-6,7- dihydropyrazolo[1,5-a]pyrimidin- 4(5H)-yl)-2-cyano-7- methylquinoxalin-5-yl)ethyl)amino)-6- chloropicolinic acid | 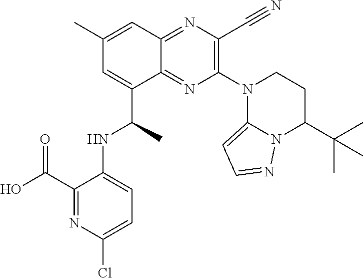 [SMILES: Cc1cc([C@@H](C)Nc2ccc(Cl)nc2C(=O)O)c2nc(N3CCC(C(C)(C)C)n4nccc43)c(C#N)nc2c1] |
| 658 | (R)-6-chloro-3-((1-(2-cyano-3-(6,7- dihydrothiazolo[4,5-c]pyridin-5(4H)- yl)-7-methylquinoxalin-5- yl)ethyl)amino)picolinic acid | 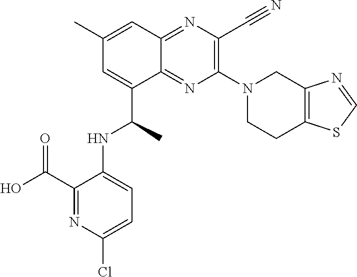 [SMILES: Cc1cc([C@@H](C)Nc2ccc(Cl)nc2C(=O)O)c2nc(N3CCc4scnc4C3)c(C#N)nc2c1] |
| 659 | (R)-6-chloro-3-((1-(2-cyano-7-methyl- 3-(2-methyl-6,7-dihydrothiazolo[4,5- c]pyridin-5(4H)-yl)quinoxalin-5- yl)ethyl)amino)picolinic acid | 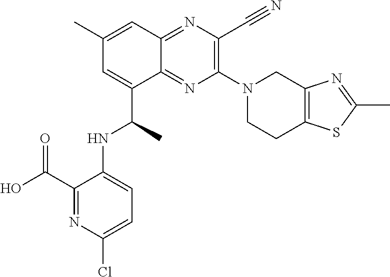 [SMILES: Cc1cc([C@@H](C)Nc2ccc(Cl)nc2C(=O)O)c2nc(N3CCc4sc(C)nc4C3)c(C#N)nc2c1] |
| 660 | (R)-6-chloro-3-((1-(2-cyano-7-methyl- 3-(1-methyl-1,4,5,7-tetrahydro-6H- pyrazolo[3,4-c]pyridin-6-yl)quinoxalin- 5-yl)ethyl) amino) picolinic acid | 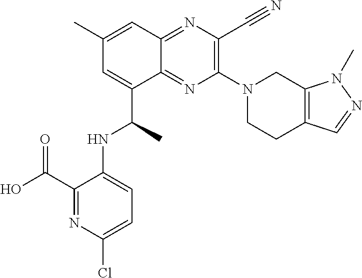 [SMILES: Cc1cc([C@@H](C)Nc2ccc(Cl)nc2C(=O)O)c2nc(N3CCc4cnn(C)c4C3)c(C#N)nc2c1] |
| 661 | (R)-6-chloro-3-((1-(2-cyano-7-methyl- 3-(5,6,8,9-tetrahydro-7H-imidazo[1,2- d][1,4]diazepin-7-yl)quinoxalin-5- yl)ethyl)amino)picolinic acid | 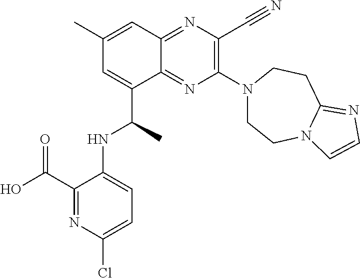 [SMILES: Cc1cc([C@@H](C)Nc2ccc(Cl)nc2C(=O)O)c2nc(N3CCc4nccn4CC3)c(C#N)nc2c1] |
| 662 | (R)-6-chloro-3-((1-(2-cyano-3-(1,1- dioxidothiomorpholino)-7- methylquinoxalin-5- yl)ethyl)amino)picolinic acid | 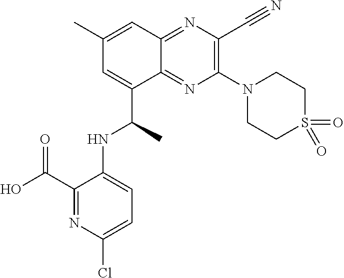 [SMILES: Cc1cc([C@@H](C)Nc2ccc(Cl)nc2C(=O)O)c2nc(N3CCS(=O)(=O)CC3)c(C#N)nc2c1] |
| 663 | (R)-6-chloro-3-((1-(2-cyano-7-methyl- 3-(4-(thiazol-2-yl)piperazin-1- yl)quinoxalin-5- yl)ethyl)amino)picolinic acid | 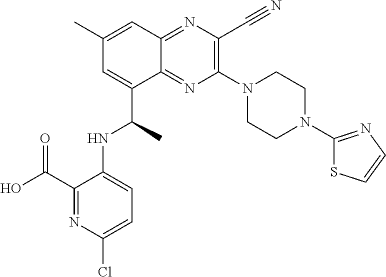 [SMILES: Cc1cc([C@@H](C)Nc2ccc(Cl)nc2C(=O)O)c2nc(N3CCN(c4nccs4)CC3)c(C#N)nc2c1] |
| 664 | (R)-6-chloro-3-((1-(2-cyano-7-methyl- 3-(4-(pyridin-2-yl)piperazin-1- yl)quinoxalin-5- yl)ethyl)amino)picolinic acid | 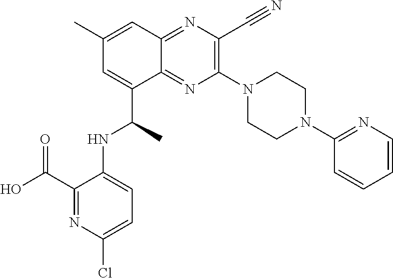 [SMILES: Cc1cc([C@@H](C)Nc2ccc(Cl)nc2C(=O)O)c2nc(N3CCN(c4ccccn4)CC3)c(C#N)nc2c1] |
| 665 | (R)-6-chloro-3-((1-(2-cyano-7-methyl- 3-(4-(5- (trifluoromethyl)benzo[d]thiazol-2- yl)piperazin-1-yl)quinoxalin-5- yl)ethyl)amino)picolinic acid | 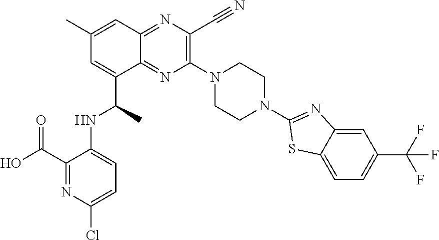 [SMILES: Cc1cc([C@@H](C)Nc2ccc(Cl)nc2C(=O)O)c2nc(N3CCN(c4nc5cc(C(F)(F)F)ccc5s4)CC3)c(C#N)nc2c1] |
| 666 | (R)-6-chloro-3-((1-(2-cyano-7-methyl- 3-(4-(4-methylthiazol-2-yl)piperazin- 1-yl)quinoxalin-5- yl)ethyl)amino)picolinic acid | 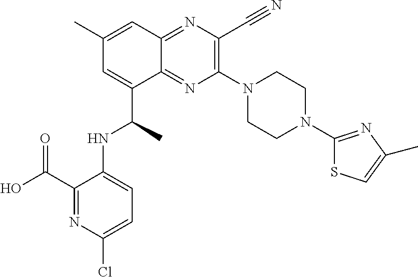 [SMILES: Cc1cc([C@@H](C)Nc2ccc(Cl)nc2C(=O)O)c2nc(N3CCN(c4nc(C)cs4)CC3)c(C#N)nc2c1] |
| 667 | (R)-6-chloro-3-((1-(2-cyano-7-methyl- 3-(4-(pyridin-3-yl)piperazin-1- yl)quinoxalin-5- yl)ethyl)amino)picolinic acid | 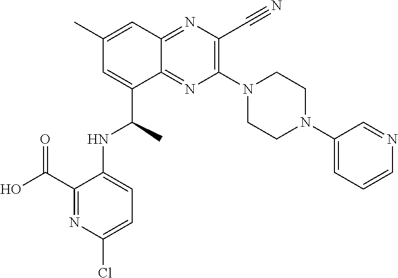 [SMILES: Cc1cc([C@@H](C)Nc2ccc(Cl)nc2C(=O)O)c2nc(N3CCN(c4cccnc4)CC3)c(C#N)nc2c1] |
| 668 | (R)-6-chloro-3-((1-(2-cyano-7-methyl- 3-(4-(1-methyl-3-(trifluoromethyl)-1H- pyrazol-4-yl)piperazin-1-yl)quinoxalin- 5-yl)ethyl)amino)picolinic acid | 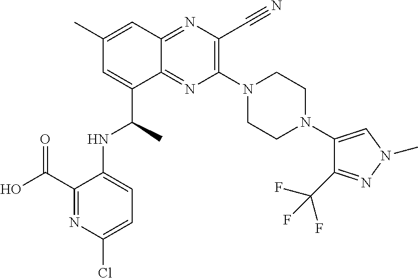 [SMILES: Cc1cc([C@@H](C)Nc2ccc(Cl)nc2C(=O)O)c2nc(N3CCN(c4cn(C)nc4C(F)(F)F)CC3)c(C#N)nc2c1] |
| 669 | (R)-6-chloro-3-((1-(2-cyano-3-(4-(3- cyano-1-methyl-1H-pyrazol-4- yl)piperazin-1-yl)-7-methylquinoxalin- 5-yl)ethyl)amino)picolinic acid | 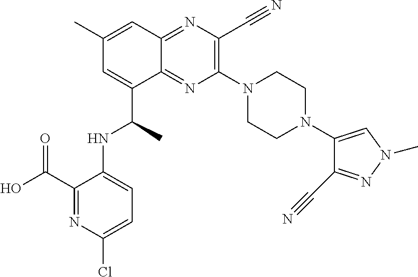 [SMILES: Cc1cc([C@@H](C)Nc2ccc(Cl)nc2C(=O)O)c2nc(N3CCN(c4cn(C)nc4C#N)CC3)c(C#N)nc2c1] |
| 670 | (R)-6-chloro-3-((1-(2-cyano-7-methyl- 3-(4-(1-methyl-1H-pyrazol-4- yl)piperazin-1-yl)quinoxalin-5- yl)ethyl)amino)picolinic acid | 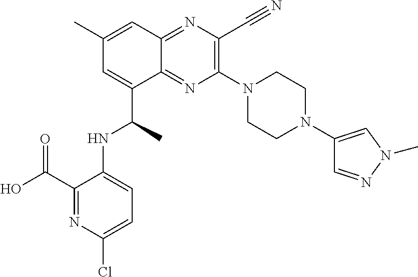 [SMILES: Cc1cc([C@@H](C)Nc2ccc(Cl)nc2C(=O)O)c2nc(N3CCN(c4cnn(C)c4)CC3)c(C#N)nc2c1] |
| 671 | (R)-6-chloro-3-((1-(2-cyano-7-methyl- 3-(4-(pyridin-4-yl)piperazin-1- yl)quinoxalin-5- yl)ethyl)amino)picolinic acid | 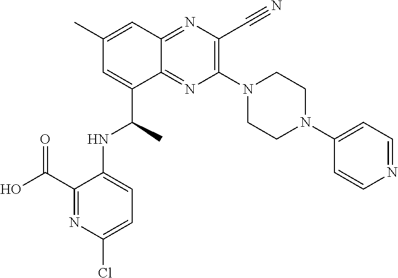 [SMILES: Cc1cc([C@@H](C)Nc2ccc(Cl)nc2C(=O)O)c2nc(N3CCN(c4ccncc4)CC3)c(C#N)nc2c1] |
| 672 | (R)-6-chloro-3-((1-(2-cyano-7-methyl- 3-(4-phenylpiperazin-1-yl)quinoxalin- 5-yl)ethyl)amino)picolinic acid | 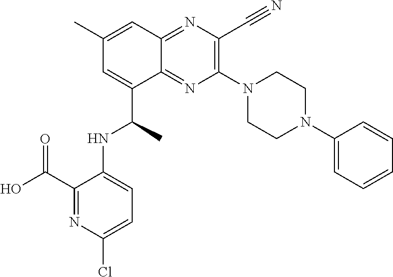 [SMILES: Cc1cc([C@@H](C)Nc2ccc(Cl)nc2C(=O)O)c2nc(N3CCN(c4ccccc4)CC3)c(C#N)nc2c1] |
| 673 | (R)-3-((1-(3-(4-(4- (aminomethyl)phenyl)piperazin-1-yl)- 2-cyano-7-methylquinoxalin-5- yl)ethyl)amino)-6-chloropicolinic acid | 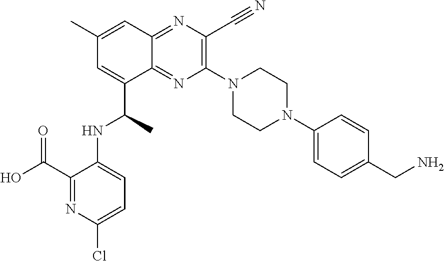 [SMILES: Cc1cc([C@@H](C)Nc2ccc(Cl)nc2C(=O)O)c2nc(N3CCN(c4ccc(CN)cc4)CC3)c(C#N)nc2c1] |
| 674 | (R)-6-chloro-3-((1-(2-cyano-7-methyl- 3-(4-(3- (trifluoromethyl)phenyl)piperazin-1- yl)quinoxalin-5- yl)ethyl)amino)picolinic acid | 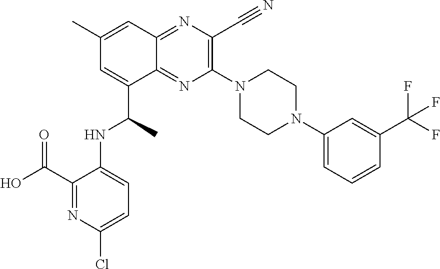 [SMILES: Cc1cc([C@@H](C)Nc2ccc(Cl)nc2C(=O)O)c2nc(N3CCN(c4cccc(C(F)(F)F)c4)CC3)c(C#N)nc2c1] |
| 675 | (R)-6-chloro-3-((1-(2-cyano-3-(4-(3- cyanophenyl)piperazin-1-yl)-7- methylquinoxalin-5- yl)ethyl)amino)picolinic acid | 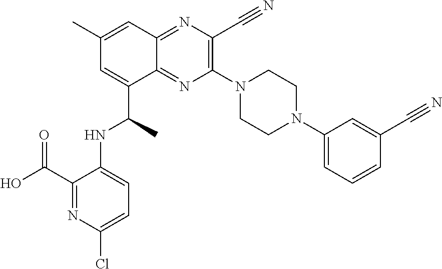 [SMILES: Cc1cc([C@@H](C)Nc2ccc(Cl)nc2C(=O)O)c2nc(N3CCN(c4cccc(C#N)c4)CC3)c(C#N)nc2c1] |
| 676 | (R)-6-chloro-3-((1-(2-cyano-3-(4-(2,6- dimethylphenyl)piperazin-1-yl)-7- methylquinoxalin-5- yl)ethyl)amino)picolinic acid | 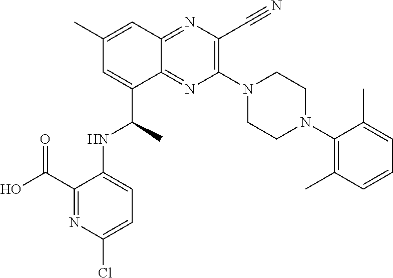 [SMILES: Cc1cc([C@@H](C)Nc2ccc(Cl)nc2C(=O)O)c2nc(N3CCN(c4c(C)cccc4C)CC3)c(C#N)nc2c1] |
| 677 | (R)-6-chloro-3-((1-(2-cyano-7-methyl- 3-(4-(o-tolyl)piperazin-1-yl)quinoxalin- 5-yl)ethyl)amino) picolinic acid | 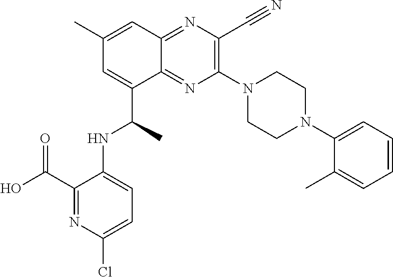 [SMILES: Cc1cc([C@@H](C)Nc2ccc(Cl)nc2C(=O)O)c2nc(N3CCN(c4ccccc4C)CC3)c(C#N)nc2c1] |
| 678 | (R)-6-chloro-3-((1-(2-cyano-3-(4-(3- cyano-2-methylphenyl)piperazin-1- yl)-7-methylquinoxalin-5- yl)ethyl)amino)picolinic acid | 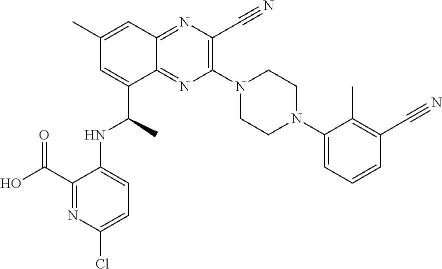 [SMILES: Cc1cc([C@@H](C)Nc2ccc(Cl)nc2C(=O)O)c2nc(N3CCN(c4cccc(C#N)c4C)CC3)c(C#N)nc2c1] |
| 679 | (R)-6-chloro-3-((1-(2-cyano-3-(4-(2- cyanopyridin-3-yl)piperazin-1-yl)-7- methylquinoxalin-5- yl)ethyl)amino)picolinic acid | 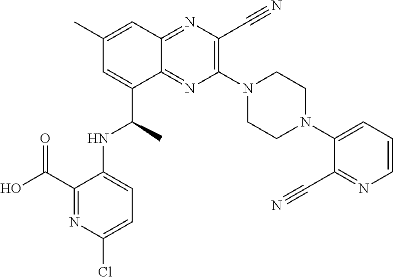 [SMILES: Cc1cc([C@@H](C)Nc2ccc(Cl)nc2C(=O)O)c2nc(N3CCN(c4cccnc4C#N)CC3)c(C#N)nc2c1] |
| 680 | (R)-6-chloro-3-((1-(2-cyano-3-(4-(5- cyanopyrimidin-2-yl)piperazin-1-yl)-7- methylquinoxalin-5- yl)ethyl)amino)picolinic acid | 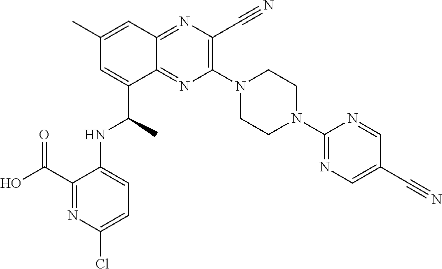 [SMILES: Cc1cc([C@@H](C)Nc2ccc(Cl)nc2C(=O)O)c2nc(N3CCN(c4ncc(C#N)cn4)CC3)c(C#N)nc2c1] |
| 681 | (R)-6-chloro-3-((1-(2-cyano-3-(4-(3- cyano-1-methyl-1H-indazol-7- yl)piperazin-1-yl)-7-methylquinoxalin- 5-yl)ethyl)amino)picolinic acid | 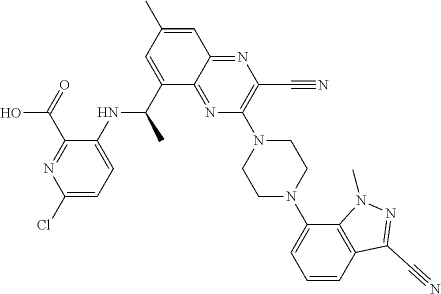 [SMILES: Cc1cc([C@@H](C)Nc2ccc(Cl)nc2C(=O)O)c2nc(N3CCN(c4cccc5c(C#N)nn(C)c45)CC3)c(C#N)nc2c1] |
| 682 | (R)-6-chloro-3-((1-(2-cyano-3-(4-(2- fluorophenyl)piperazin-1-yl)-7- methylquinoxalin-5- yl)ethyl)amino)picolinic acid | 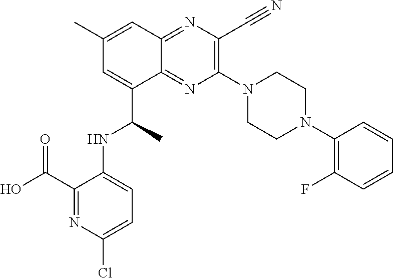 [SMILES: Cc1cc([C@@H](C)Nc2ccc(Cl)nc2C(=O)O)c2nc(N3CCN(c4ccccc4F)CC3)c(C#N)nc2c1] |
| 683 | (R)-6-chloro-3-((1-(3-(4-(2- chlorophenyl)piperazin-1-yl)-2-cyano- 7-methylquinoxalin-5- yl)ethyl)amino)picolinic acid | 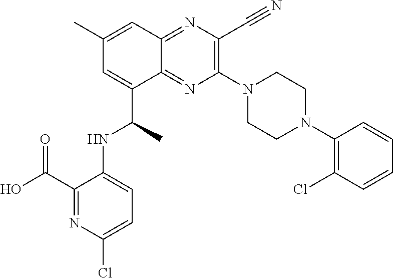 [SMILES: Cc1cc([C@@H](C)Nc2ccc(Cl)nc2C(=O)O)c2nc(N3CCN(c4ccccc4Cl)CC3)c(C#N)nc2c1] |
| 684 | (R)-6-chloro-3-((1-(2-cyano-3-(4-(2- cyanophenyl)piperazin-1-yl)-7- methylquinoxalin-5- yl)ethyl)amino)picolinic acid | 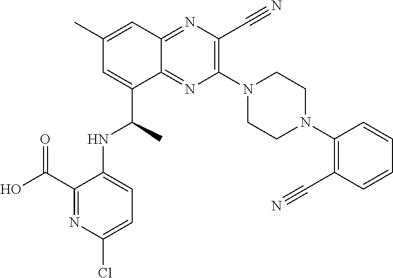 [SMILES: Cc1cc([C@@H](C)Nc2ccc(Cl)nc2C(=O)O)c2nc(N3CCN(c4ccccc4C#N)CC3)c(C#N)nc2c1] |
| 685 | (R)-6-chloro-3-((1-(2-cyano-3-(4-(2,4- difluorophenyl)piperazin-1-yl)-7- methylquinoxalin-5- yl)ethyl)amino)picolinic acid | 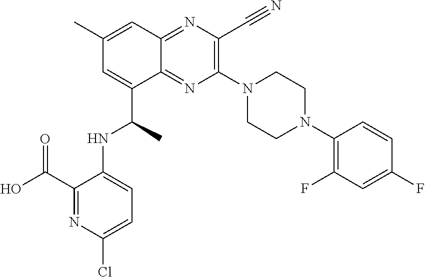 [SMILES: Cc1cc([C@@H](C)Nc2ccc(Cl)nc2C(=O)O)c2nc(N3CCN(c4ccc(F)cc4F)CC3)c(C#N)nc2c1] |
| 686 | (R)-6-chloro-3-((1-(2-cyano-3-(4-(4- cyano-2-fluorophenyl)piperazin-1-yl)- 7-methylquinoxalin-5- yl)ethyl)amino)picolinic acid | 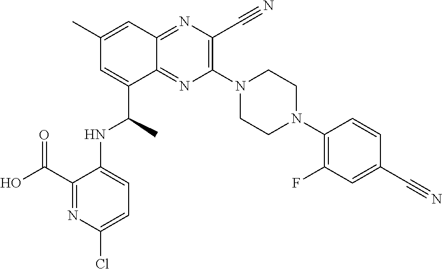 [SMILES: Cc1cc([C@@H](C)Nc2ccc(Cl)nc2C(=O)O)c2nc(N3CCN(c4ccc(C#N)cc4F)CC3)c(C#N)nc2c1] |
| 687 | (R)-6-chloro-3-((1-(2-cyano-7-methyl- 3-(4-(4-(N- methylsulfamoyl)phenyl)piperazin-1- yl)quinoxalin-5- yl)ethyl)amino)picolinic acid | 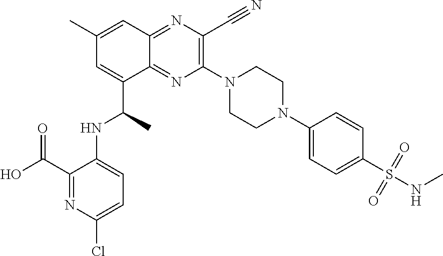 [SMILES: CNS(=O)(=O)c1ccc(N2CCN(c3nc4c([C@@H](C)Nc5ccc(Cl)nc5C(=O)O)cc(C)cc4nc3C#N)CC2)cc1] |
| 688 | (R)-6-chloro-3-((1-(2-cyano-3-(4-(4- fluorophenyl)piperazin-1-yl)-7- methylquinoxalin-5- yl)ethyl)amino)picolinic acid | 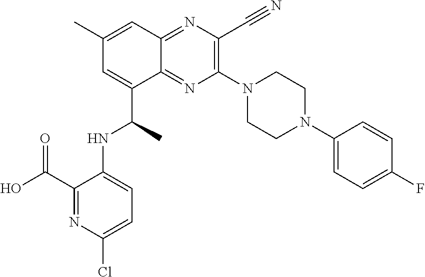 [SMILES: Cc1cc([C@@H](C)Nc2ccc(Cl)nc2C(=O)O)c2nc(N3CCN(c4ccc(F)cc4)CC3)c(C#N)nc2c1] |
| 689 | (R)-6-chloro-3-((1-(2-cyano-7-methyl- 3-(4-(4- (methylcarbamoyl)phenyl)piperazin- 1-yl)quinoxalin-5- yl)ethyl)amino)picolinic acid | 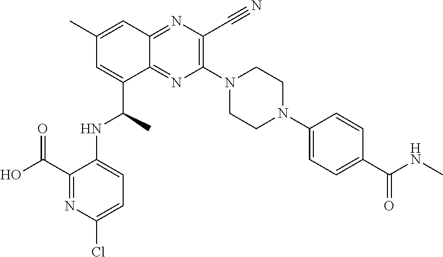 [SMILES: CNC(=O)c1ccc(N2CCN(c3nc4c([C@@H](C)Nc5ccc(Cl)nc5C(=O)O)cc(C)cc4nc3C#N)CC2)cc1] |
| 690 | (R)-3-((1-(3-(4-(4- carbamoylphenyl)piperazin-1-yl)-2- cyano-7-methylquinoxalin-5- yl)ethyl)amino)-6-chloropicolinic acid | 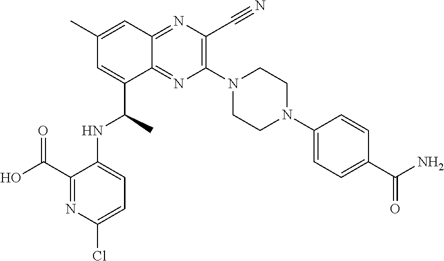 [SMILES: Cc1cc([C@@H](C)Nc2ccc(Cl)nc2C(=O)O)c2nc(N3CCN(c4ccc(C(N)=O)cc4)CC3)c(C#N)nc2c1] |
| 691 | (R)-6-chloro-3-((1-(2-cyano-3-(4-(4- cyanophenyl)piperazin-1-yl)-7- methylquinoxalin-5- yl)ethyl)amino)picolinic acid | 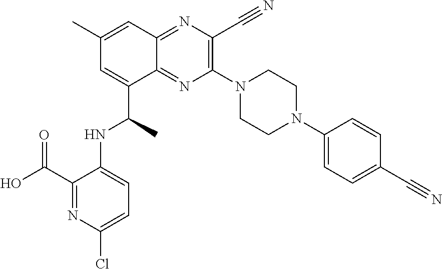 [SMILES: Cc1cc([C@@H](C)Nc2ccc(Cl)nc2C(=O)O)c2nc(N3CCN(c4ccc(C#N)cc4)CC3)c(C#N)nc2c1] |
| 692 | (R)-6-chloro-3-((1-(2-cyano-3-(4-(4- cyano-2-methylphenyl)piperazin-1- yl)-7-methylquinoxalin-5- yl)ethyl)amino)picolinic acid | 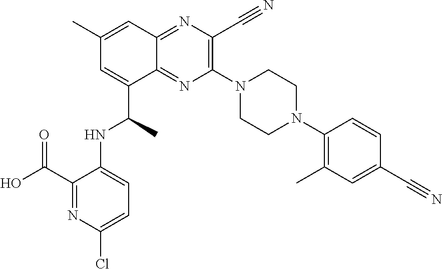 [SMILES: Cc1cc([C@@H](C)Nc2ccc(Cl)nc2C(=O)O)c2nc(N3CCN(c4ccc(C#N)cc4C)CC3)c(C#N)nc2c1] |
| 693 | (R)-6-chloro-3-((1-(2-cyano-3-(4-(8- cyanoisoquinolin-5-yl)piperazin-1-yl)- 7-methylquinoxalin-5- yl)ethyl)amino)picolinic acid | 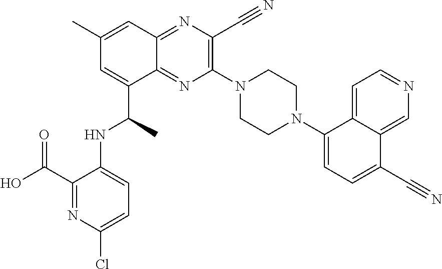 [SMILES: Cc1cc([C@@H](C)Nc2ccc(Cl)nc2C(=O)O)c2nc(N3CCN(c4ccc(C#N)c5cnccc45)CC3)c(C#N)nc2c1] |
| 694 | (R)-6-chloro-3-((1-(2-cyano-3-(4-(4- cyano-7-hydroxynaphthalen-1- yl)piperazin-1-yl)-7-methylquinoxalin- 5-yl)ethyl)amino)picolinic acid | 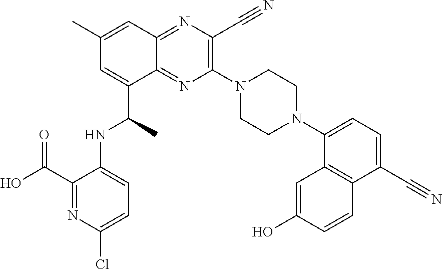 [SMILES: Cc1cc([C@@H](C)Nc2ccc(Cl)nc2C(=O)O)c2nc(N3CCN(c4ccc(C#N)c5ccc(O)cc45)CC3)c(C#N)nc2c1] |
| 695 | (R)-6-chloro-3-((1-(2-cyano-3-(4-(4- cyano-6-hydroxynaphthalen-1- yl)piperazin-1-yl)-7-methylquinoxalin- 5-yl)ethyl)amino)picolinic acid | 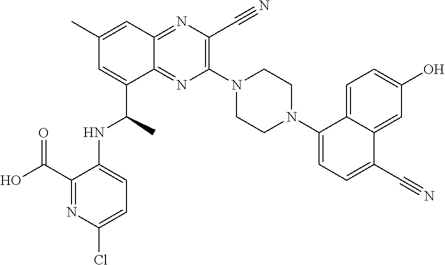 [SMILES: Cc1cc([C@@H](C)Nc2ccc(Cl)nc2C(=O)O)c2nc(N3CCN(c4ccc(C#N)c5cc(O)ccc45)CC3)c(C#N)nc2c1] |
| 696 | (R)-6-chloro-3-((1-(2-cyano-3-(4-(4,6- dicyanonaphthalen-1-yl)piperazin-1- yl)-7-methylquinoxalin-5- yl)ethyl)amino)picolinic acid | 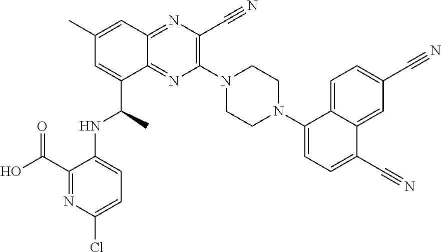 [SMILES: Cc1cc([C@@H](C)Nc2ccc(Cl)nc2C(=O)O)c2nc(N3CCN(c4ccc(C#N)c5cc(C#N)ccc45)CC3)c(C#N)nc2c1] |
| 697 | (R)-6-chloro-3-((1-(2-cyano-7-methyl- 3-(4-(methylsulfonyl)piperazin-1- yl)quinoxalin-5- yl)ethyl)amino)picolinic acid | 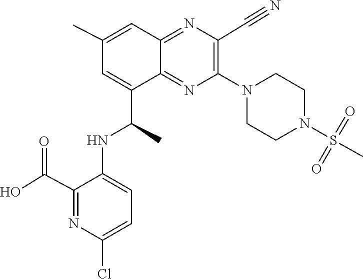 [SMILES: Cc1cc([C@@H](C)Nc2ccc(Cl)nc2C(=O)O)c2nc(N3CCN(S(C)(=O)=O)CC3)c(C#N)nc2c1] |
| 698 | (R)-6-chloro-3-((1-(2-cyano-7-methyl- 3-(4-(oxetan-3-yl)piperazin-1- yl)quinoxalin-5- yl)ethyl)amino)picolinic acid | 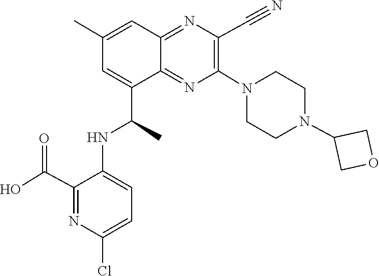 [SMILES: Cc1cc([C@@H](C)Nc2ccc(Cl)nc2C(=O)O)c2nc(N3CCN(C4COC4)CC3)c(C#N)nc2c1] |
| 699 | (R)-6-chloro-3-((1-(2-cyano-7-methyl- 3-(4-(tetrahydro-2H-pyran-4- yl)piperazin-1-yl)quinoxalin-5- yl)ethyl)amino)picolinic acid | 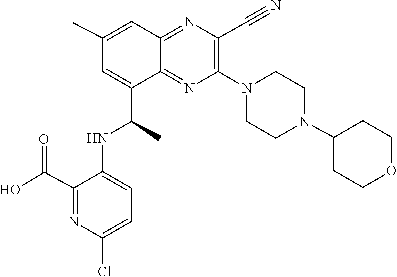 [SMILES: Cc1cc([C@@H](C)Nc2ccc(Cl)nc2C(=O)O)c2nc(N3CCN(C4CCOCC4)CC3)c(C#N)nc2c1] |
| 700 | (R)-6-chloro-3-((1-(2-cyano-3-(4- cyclopropylpiperazin-1-yl)-7- methylquinoxalin-5- yl)ethyl)amino)picolinic acid | 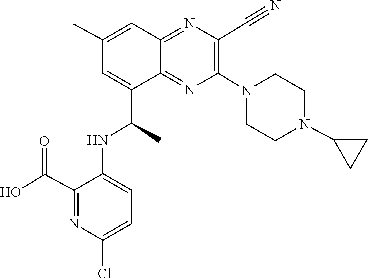 [SMILES: Cc1cc([C@@H](C)Nc2ccc(Cl)nc2C(=O)O)c2nc(N3CCN(C4CC4)CC3)c(C#N)nc2c1] |
| 701 | (R)-6-chloro-3-((1-(2-cyano-7-methyl- 3-(4-(1-methyl-1H-pyrazole-4- carbonyl)piperazin-1-yl)quinoxalin-5- yl)ethyl)amino)picolinic acid | 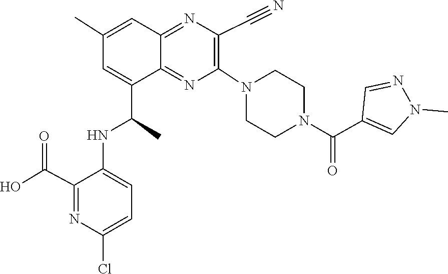 [SMILES: Cc1cc([C@@H](C)Nc2ccc(Cl)nc2C(=O)O)c2nc(N3CCN(C(=O)c4cnn(C)c4)CC3)c(C#N)nc2c1] |
| 702 | (R)-3-((1-(3-(4-benzoylpiperazin-1- yl)-2-cyano-7-methylquinoxalin-5- yl)ethyl)amino)-6-chloropicolinic acid | 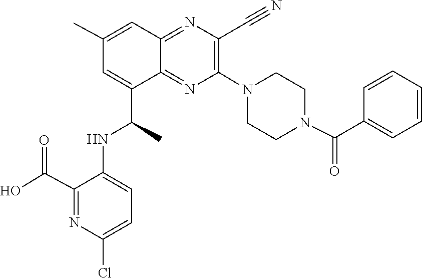 [SMILES: Cc1cc([C@@H](C)Nc2ccc(Cl)nc2C(=O)O)c2nc(N3CCN(C(=O)c4ccccc4)CC3)c(C#N)nc2c1] |
| 703 | (R)-6-chloro-3-((1-(3-(4-(4- chlorobenzoyl)piperazin-1-yl)-2- cyano-7-methylquinoxalin-5- yl)ethyl)amino)picolinic acid | 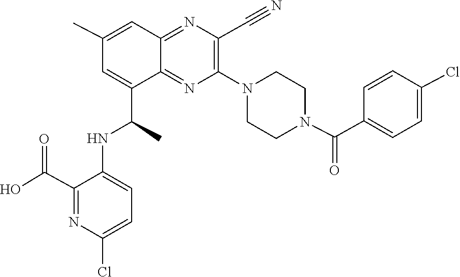 [SMILES: Cc1cc([C@@H](C)Nc2ccc(Cl)nc2C(=O)O)c2nc(N3CCN(C(=O)c4ccc(Cl)cc4)CC3)c(C#N)nc2c1] |
| 704 | (R)-6-chloro-3-((1-(2-cyano-3-(4- (cyclopentanecarbonyl)piperazin-1- yl)-7-methylquinoxalin-5- yl)ethyl)amino)picolinic acid | 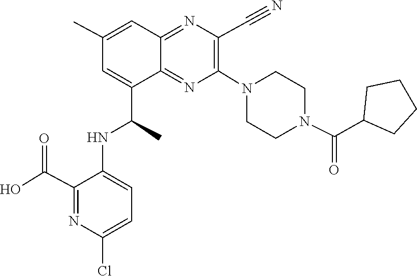 [SMILES: Cc1cc([C@@H](C)Nc2ccc(Cl)nc2C(=O)O)c2nc(N3CCN(C(=O)C4CCCC4)CC3)c(C#N)nc2c1] |
| 705 | (R)-6-chloro-3-((1-(2-cyano-3-(4- (cyclopropanecarbonyl)piperazin-1- yl)-7-methylquinoxalin-5- yl)ethyl)amino)picolinic acid | 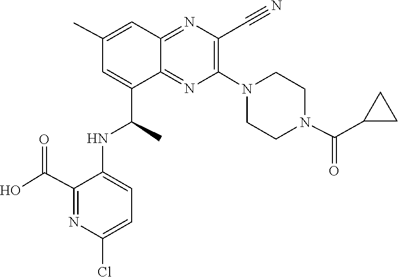 [SMILES: Cc1cc([C@@H](C)Nc2ccc(Cl)nc2C(=O)O)c2nc(N3CCN(C(=O)C4CC4)CC3)c(C#N)nc2c1] |
| 706 | (R)-6-chloro-3-((1-(2-cyano-7-methyl- 3-(4-(3-(trifluoromethyl)-1H-pyrazol-1- yl)piperidin-1-yl)quinoxalin-5- yl)ethyl)amino)picolinic acid | 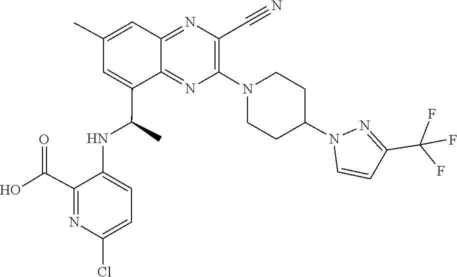 [SMILES: Cc1cc([C@@H](C)Nc2ccc(Cl)nc2C(=O)O)c2nc(N3CCC(n4ccc(C(F)(F)F)n4)CC3)c(C#N)nc2c1] |
| 707 | (R)-6-chloro-3-((1-(2-cyano-7-methyl- 3-(4-(1-methyl-1H-pyrazol-3- yl)piperidin-1-yl)quinoxalin-5- yl)ethyl)amino)picolinic acid | 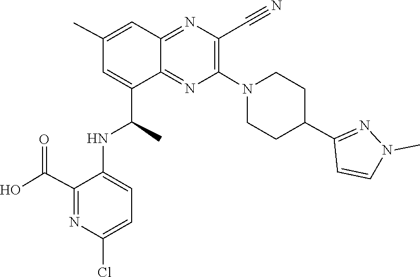 [SMILES: Cc1cc([C@@H](C)Nc2ccc(Cl)nc2C(=O)O)c2nc(N3CCC(c4ccn(C)n4)CC3)c(C#N)nc2c1] |
| 708 | (R)-3-((1-(3-(4-(1H-pyrazol-3- yl)piperidin-1-yl)-2-cyano-7- methylquinoxalin-5-yl)ethyl)amino)-6- chloropicolinic acid | 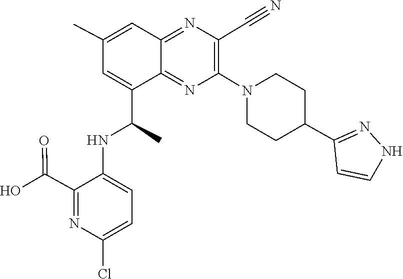 [SMILES: Cc1cc([C@@H](C)Nc2ccc(Cl)nc2C(=O)O)c2nc(N3CCC(c4cc[nH]n4)CC3)c(C#N)nc2c1] |
| 709 | (R)-6-chloro-3-((1-(2-cyano-3-(4-(4- methoxyphenyl)piperidin-1-yl)-7- methylquinoxalin-5- yl)ethyl)amino)picolinic acid | 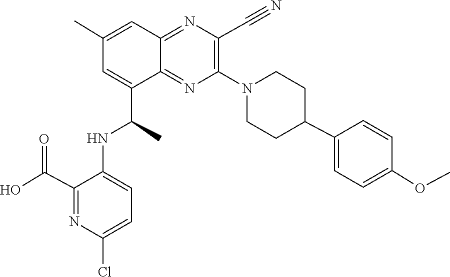 [SMILES: COc1ccc(C2CCN(c3nc4c([C@@H](C)Nc5ccc(Cl)nc5C(=O)O)cc(C)cc4nc3C#N)CC2)cc1] |
| 710 | (R)-6-chloro-3-((1-(2-cyano-3-(4- methoxypiperidin-1-yl)-7- methylquinoxalin-5- yl)ethyl)amino)picolinic acid | 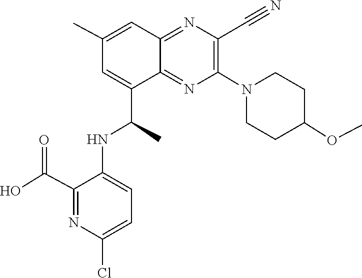 [SMILES: COC1CCN(c2nc3c([C@@H](C)Nc4ccc(Cl)nc4C(=O)O)cc(C)cc3nc2C#N)CC1] |
| 711 | (R)-6-chloro-3-((1-(2-cyano-3-(4- hydroxypiperidin-1-yl)-7- methylquinoxalin-5- yl)ethyl)amino)picolinic acid | 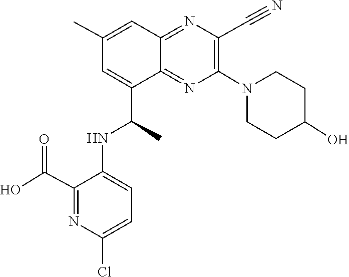 [SMILES: Cc1cc([C@@H](C)Nc2ccc(Cl)nc2C(=O)O)c2nc(N3CCC(O)CC3)c(C#N)nc2c1] |
| 712 | (R)-3-((1-(3-([1,4′-bipiperidin]-1′-yl)-2- cyano-7-methylquinoxalin-5- yl)ethyl)amino)-6-chloropicolinic acid | 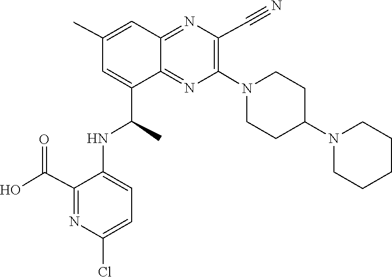 [SMILES: Cc1cc([C@@H](C)Nc2ccc(Cl)nc2C(=O)O)c2nc(N3CCC(N4CCCCC4)CC3)c(C#N)nc2c1] |
| 713 | (R)-6-chloro-3-((1-(2-cyano-3-(4,4- difluoro-[1,4′-bipiperidin]-1′-yl)-7- methylquinoxalin-5- yl)ethyl)amino)picolinic acid | 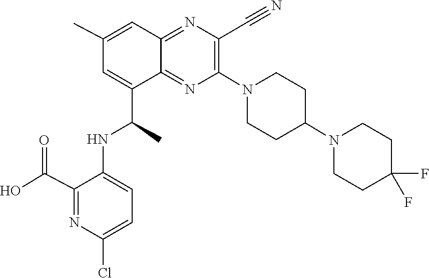 [SMILES: Cc1cc([C@@H](C)Nc2ccc(Cl)nc2C(=O)O)c2nc(N3CCC(N4CCC(F)(F)CC4)CC3)c(C#N)nc2c1] |
| 714 | (R)-6-chloro-3-((1-(2-cyano-3-(4- (dimethylamino)piperidin-1-yl)-7- methylquinoxalin-5- yl)ethyl)amino)picolinic acid | 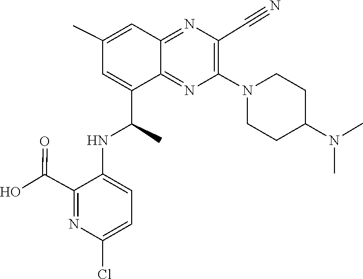 [SMILES: Cc1cc([C@@H](C)Nc2ccc(Cl)nc2C(=O)O)c2nc(N3CCC(N(C)C)CC3)c(C#N)nc2c1] |
| 715 | (R)-6-chloro-3-((1-(2-cyano-3-(4- cyclopentylpiperidin-1-yl)-7- methylquinoxalin-5- yl)ethyl)amino)picolinic acid | 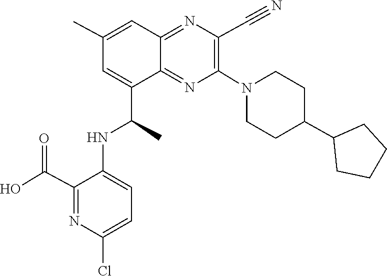 [SMILES: Cc1cc([C@@H](C)Nc2ccc(Cl)nc2C(=O)O)c2nc(N3CCC(C4CCCC4)CC3)c(C#N)nc2c1] |
| 716 | (R)-6-chloro-3-((1-(2-cyano-3-(4- cyclobutylpiperidin-1-yl)-7- methylquinoxalin-5- yl)ethyl)amino)picolinic acid | 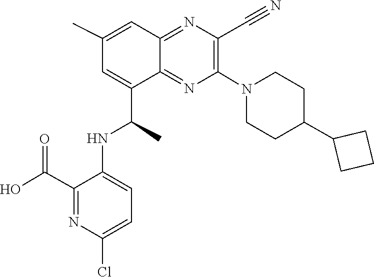 [SMILES: Cc1cc([C@@H](C)Nc2ccc(Cl)nc2C(=O)O)c2nc(N3CCC(C4CCC4)CC3)c(C#N)nc2c1] |
| 717 | (R)-6-chloro-3-((1-(2-cyano-7-methyl- 3-(4-(trifluoromethyl)piperidin-1- yl)quinoxalin-5- yl)ethyl)amino)picolinic acid | 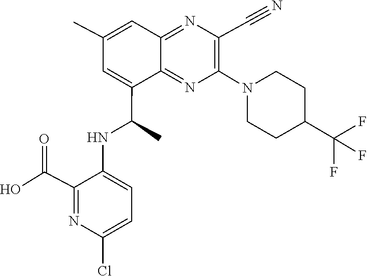 [SMILES: Cc1cc([C@@H](C)Nc2ccc(Cl)nc2C(=O)O)c2nc(N3CCC(C(F)(F)F)CC3)c(C#N)nc2c1] |
| 718 | (R)-6-chloro-3-((1-(2-cyano-3-(4- cyanopiperidin-1-yl)-7- methylquinoxalin-5- yl)ethyl)amino)picolinic acid | 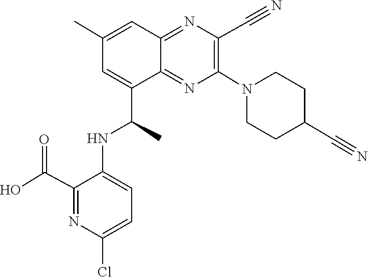 [SMILES: Cc1cc([C@@H](C)Nc2ccc(Cl)nc2C(=O)O)c2nc(N3CCC(C#N)CC3)c(C#N)nc2c1] |
| 719 | (R)-6-chloro-3-((1-(2-cyano-7-methyl- 3-(1-oxa-8-azaspiro[4.5]decan-8- yl)quinoxalin-5- yl)ethyl)amino)picolinic acid | 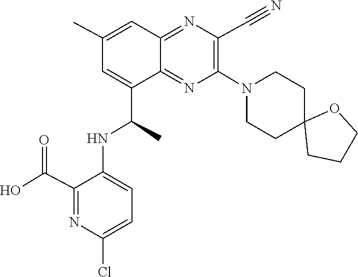 [SMILES: Cc1cc([C@@H](C)Nc2ccc(Cl)nc2C(=O)O)c2nc(N3CCC4(CCCO4)CC3)c(C#N)nc2c1] |
| 720 | (R)-6-chloro-3-((1-(2-cyano-3-(1,1- difluoro-6-azaspiro[2.5]octan-6-yl)-7- methylquinoxalin-5- yl)ethyl)amino)picolinic acid | 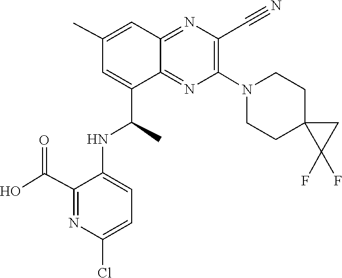 [SMILES: Cc1cc([C@@H](C)Nc2ccc(Cl)nc2C(=O)O)c2nc(N3CCC4(CC3)CC4(F)F)c(C#N)nc2c1] |
| 721 | (R)-6-chloro-3-((1-(2-cyano-3-(4- methoxy-4-methylpiperidin-1-yl)-7- methylquinoxalin-5- yl)ethyl)amino)picolinic acid | 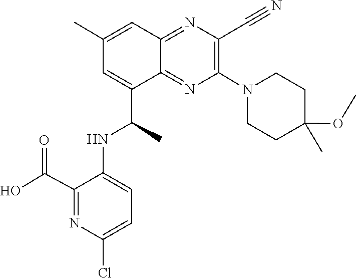 [SMILES: COC1(C)CCN(c2nc3c([C@@H](C)Nc4ccc(Cl)nc4C(=O)O)cc(C)cc3nc2C#N)CC1] |
| 722 | (R)-6-chloro-3-((1-(2-cyano-3-(4- cyano-4-methylpiperidin-1-yl)-7- methylquinoxalin-5- yl)ethyl)amino)picolinic acid | 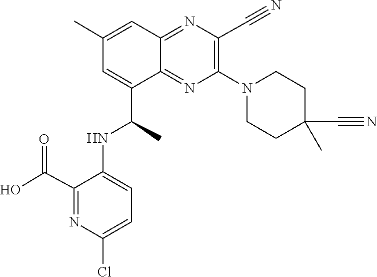 [SMILES: Cc1cc([C@@H](C)Nc2ccc(Cl)nc2C(=O)O)c2nc(N3CCC(C)(C#N)CC3)c(C#N)nc2c1] |
| 723 | 6-chloro-3-(((1R)-1-(2-cyano-7- methyl-3-(3-(pyridin-2-yl)pyrrolidin-1- yl)quinoxalin-5- yl)ethyl)amino)picolinic acid | 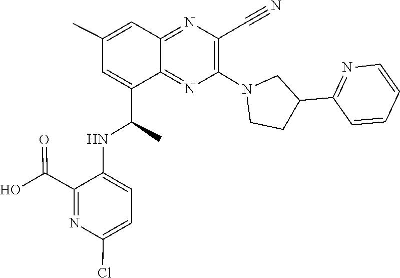 [SMILES: Cc1cc([C@@H](C)Nc2ccc(Cl)nc2C(=O)O)c2nc(N3CCC(c4ccccn4)C3)c(C#N)nc2c1] |
| 724 | 6-chloro-3-(((1R)-1-(2-cyano-7- methyl-3-(3-(pyridin-4-yl)pyrrolidin-1- yl)quinoxalin-5- yl)ethyl)amino)picolinic acid | 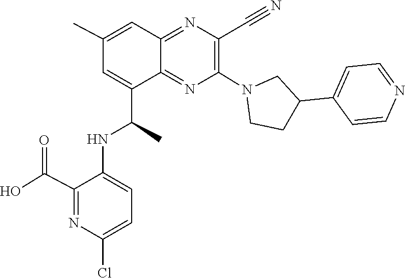 [SMILES: Cc1cc([C@@H](C)Nc2ccc(Cl)nc2C(=O)O)c2nc(N3CCC(c4ccncc4)C3)c(C#N)nc2c1] |
| 725 | 6-chloro-3-(((1R)-1-(2-cyano-7- methyl-3-(3-phenylpyrrolidin-1- yl)quinoxalin-5- yl)ethyl)amino)picolinic acid | 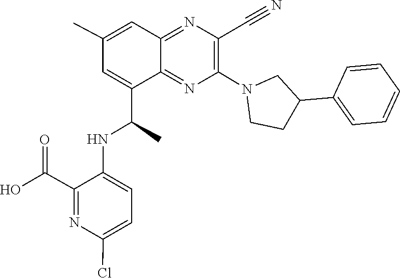 [SMILES: Cc1cc([C@@H](C)Nc2ccc(Cl)nc2C(=O)O)c2nc(N3CCC(c4ccccc4)C3)c(C#N)nc2c1] |
| 726 | 6-chloro-3-(((1R)-1-(2-cyano-3-(3- (1,3-dihydroxypropan-2-yl)pyrrolidin- 1-yl)-7-methylquinoxalin-5- yl)ethyl)amino)picolinic acid | 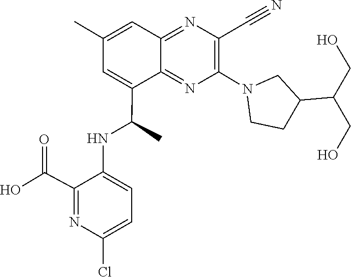 [SMILES: Cc1cc([C@@H](C)Nc2ccc(Cl)nc2C(=O)O)c2nc(N3CCC(C(CO)CO)C3)c(C#N)nc2c1] |
| 727 | 6-chloro-3-(((1R)-1-(2-cyano-7- methyl-3-(1-oxa-6- azaspiro[3.4]octan-6-yl)quinoxalin-5- yl)ethyl)amino)picolinic acid | 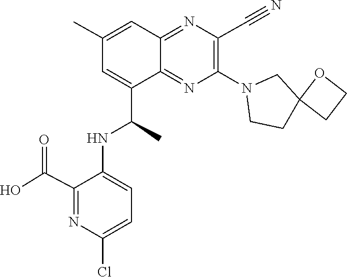 [SMILES: Cc1cc([C@@H](C)Nc2ccc(Cl)nc2C(=O)O)c2nc(N3CCC4(CCO4)C3)c(C#N)nc2c1] |
| 728 | 6-chloro-3-(((1R)-1-(2-cyano-7- methyl-3- (spiro[bicyclo[2.2.1]heptane-2,3′- pyrrolidin]-1′-yl)quinoxalin-5- yl)ethyl)amino)picolinic acid | 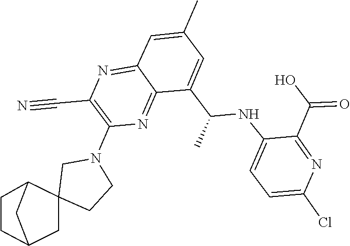 [SMILES: Cc1cc([C@@H](C)Nc2ccc(Cl)nc2C(=O)O)c2nc(N3CCC4(CC5CCC4C5)C3)c(C#N)nc2c1] |
| 729 | 6-chloro-3-(((1R)-1-(2-cyano-7- methyl-3-(7-methyl-6-oxo-2,7- diazaspiro[4.4]nonan-2-yl)quinoxalin- 5-yl)ethyl)amino)picolinic acid | 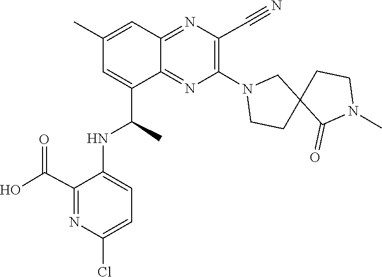 [SMILES: Cc1cc([C@@H](C)Nc2ccc(Cl)nc2C(=O)O)c2nc(N3CCC4(CCN(C)C4=O)C3)c(C#N)nc2c1] |
| 730 | 6-chloro-3-(((1R)-1-(2-cyano-3-(8,9- dihydroxy-6-oxa-2- azaspiro[4.5]decan-2-yl)-7- methylquinoxalin-5- yl)ethyl)amino)picolinic acid | 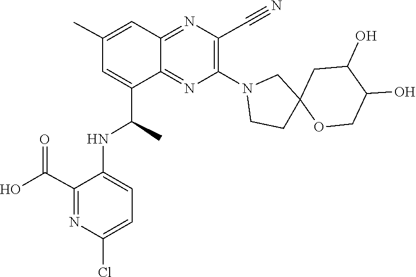 [SMILES: Cc1cc([C@@H](C)Nc2ccc(Cl)nc2C(=O)O)c2nc(N3CCC4(CC(O)C(O)CO4)C3)c(C#N)nc2c1] |
| | | C₂₆H₂₇ClN₆O₅ |
| 731 | 6-chloro-3-(((1R)-1-(2-cyano-7- methyl-3-(2-oxo-1,7- diazaspiro[4.4]nonan-7-yl)quinoxalin- 5-yl)ethyl)amino)picolinic acid | 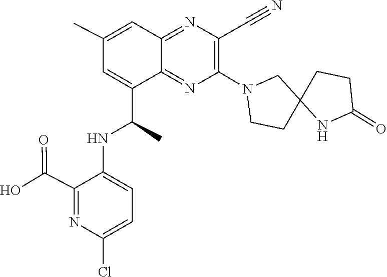 [SMILES: Cc1cc([C@@H](C)Nc2ccc(Cl)nc2C(=O)O)c2nc(N3CCC4(CCC(=O)N4)C3)c(C#N)nc2c1] |
| 732 | 6-chloro-3-(((1R)-1-(2-cyano-3-(7,7- difluoro-2-azaspiro[4.4]nonan-2-yl)-7- methylquinoxalin-5- yl)ethyl)amino)picolinic acid | 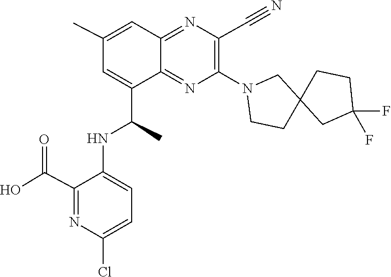 [SMILES: Cc1cc([C@@H](C)Nc2ccc(Cl)nc2C(=O)O)c2nc(N3CCC4(CCC(F)(F)C4)C3)c(C#N)nc2c1] |
| 733 | 6-chloro-3-(((1R)-1-(2-cyano-7- methyl-3-(1,1,2,2-tetrafluoro-6- azaspiro[3.4]octan-6-yl)quinoxalin-5- yl)ethyl)amino)picolinic acid | 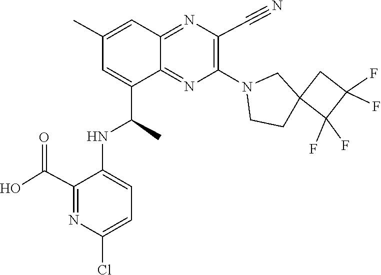 [SMILES: Cc1cc([C@@H](C)Nc2ccc(Cl)nc2C(=O)O)c2nc(N3CCC4(C3)CC(F)(F)C4(F)F)c(C#N)nc2c1] |
| 734 | 6-chloro-3-(((1R)-1-(2-cyano-3-(4-(4- cyanophenyl)-3-methylpiperazin-1- yl)-7-methylquinoxalin-5- yl)ethyl)amino)picolinic acid | 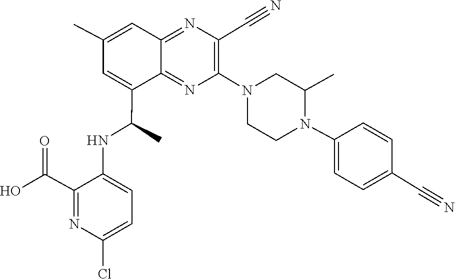 [SMILES: Cc1cc([C@@H](C)Nc2ccc(Cl)nc2C(=O)O)c2nc(N3CCN(c4ccc(C#N)cc4)C(C)C3)c(C#N)nc2c1] |
| 735 | 6-chloro-3-(((1R)-1-(2-cyano-7- methyl-3-(2-(trifluoromethyl)azetidin- 1-yl)quinoxalin-5- yl)ethyl)amino)picolinic acid | 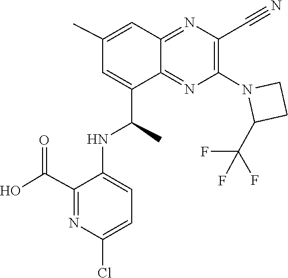 [SMILES: Cc1cc([C@@H](C)Nc2ccc(Cl)nc2C(=O)O)c2nc(N3CCC3C(F)(F)F)c(C#N)nc2c1] |
| 736 | 6-chloro-3-(((1R)-1-(2-cyano-7- methyl-3-(2-methyl-4- phenylpiperazin-1-yl)quinoxalin-5- yl)ethyl)amino)picolinic acid | 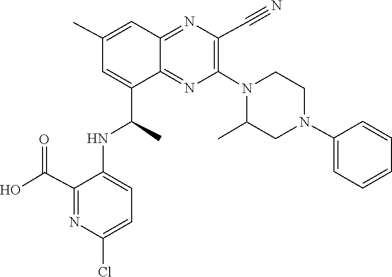 [SMILES: Cc1cc([C@@H](C)Nc2ccc(Cl)nc2C(=O)O)c2nc(N3CCN(c4ccccc4)CC3C)c(C#N)nc2c1] |
| 737 | 6-chloro-3-(((1R)-1-(2-cyano-3-(4-(3- cyanophenyl)-2-methylpiperazin-1- yl)-7-methylquinoxalin-5- yl)ethyl)amino)picolinic acid | 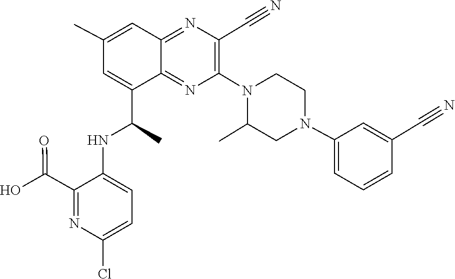 [SMILES: Cc1cc([C@@H](C)Nc2ccc(Cl)nc2C(=O)O)c2nc(N3CCN(c4cccc(C#N)c4)CC3C)c(C#N)nc2c1] |
| 738 | 6-chloro-3-(((1R)-1-(2-cyano-3-(4-(2- cyanophenyl)-2-methylpiperazin-1- yl)-7-methylquinoxalin-5- yl)ethyl)amino)picolinic acid | 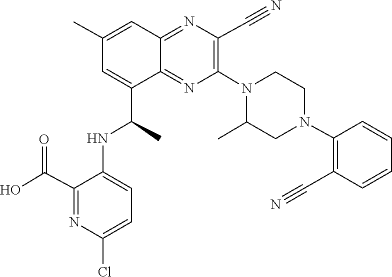 [SMILES: Cc1cc([C@@H](C)Nc2ccc(Cl)nc2C(=O)O)c2nc(N3CCN(c4ccccc4C#N)CC3C)c(C#N)nc2c1] |
| 739 | 6-chloro-3-(((1R)-1-(2-cyano-3-(4-(2- cyano-4-(trifluoromethyl)phenyl)-2- methylpiperazin-1-yl)-7- methylquinoxalin-5- yl)ethyl)amino)picolinic acid | 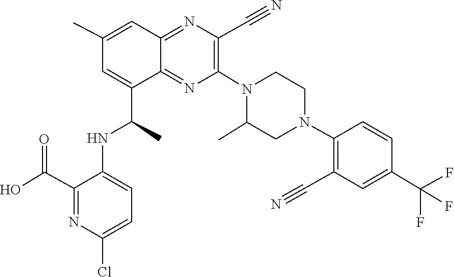 [SMILES: Cc1cc([C@@H](C)Nc2ccc(Cl)nc2C(=O)O)c2nc(N3CCN(c4ccc(C(F)(F)F)cc4C#N)CC3C)c(C#N)nc2c1] |
| 740 | 6-chloro-3-(((1R)-1-(2-cyano-3-(4-(4- fluorophenyl)-2-methylpiperazin-1-yl)- 7-methylquinoxalin-5- yl)ethyl)amino)picolinic acid | 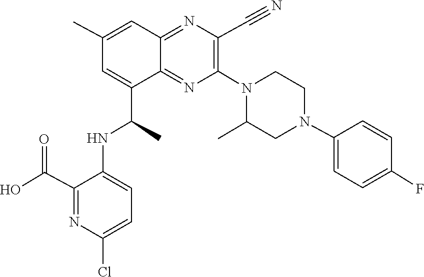 [SMILES: Cc1cc([C@@H](C)Nc2ccc(Cl)nc2C(=O)O)c2nc(N3CCN(c4ccc(F)cc4)CC3C)c(C#N)nc2c1] |
| 741 | 6-chloro-3-(((1R)-1-(2-cyano-3-(4-(4- cyanophenyl)-2-methylpiperazin-1- yl)-7-methylquinoxalin-5- yl)ethyl)amino)picolinic acid | 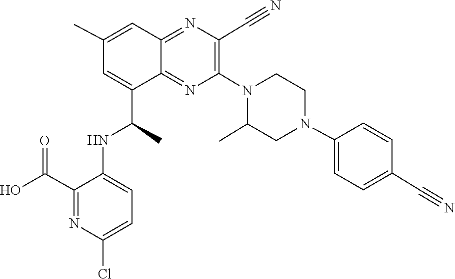 [SMILES: Cc1cc([C@@H](C)Nc2ccc(Cl)nc2C(=O)O)c2nc(N3CCN(c4ccc(C#N)cc4)CC3C)c(C#N)nc2c1] |
| 742 | 6-chloro-3-(((1R)-1-(2-cyano-3-(4-(2- cyano-5-methylphenyl)-2- methylpiperazin-1-yl)-7- methylquinoxalin-5- yl)ethyl)amino)picolinic acid | 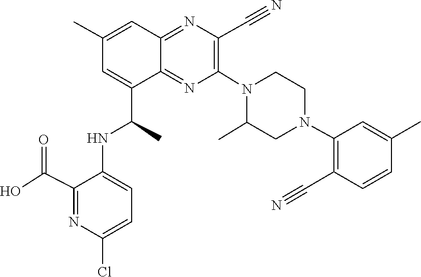 [SMILES: Cc1ccc(C#N)c(N2CCN(c3nc4c([C@@H](C)Nc5ccc(Cl)nc5C(=O)O)cc(C)cc4nc3C#N)C(C)C2)c1] |
| 743 | 6-chloro-3-(((1R)-1-(2-cyano-3-(4,4- difluoro-2-methylpiperidin-1-yl)-7- methylquinoxalin-5- yl)ethyl)amino)picolinic acid | 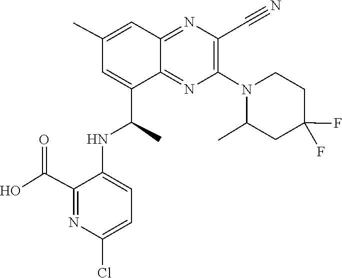 [SMILES: Cc1cc([C@@H](C)Nc2ccc(Cl)nc2C(=O)O)c2nc(N3CCC(F)(F)CC3C)c(C#N)nc2c1] |
| 744 | (R)-6-chloro-3-((1-(2-cyano-7-methyl- 3-(3,3,4,4-tetrafluoropyrrolidin-1- yl)quinoxalin-5- yl)ethyl)amino)picolinic acid | 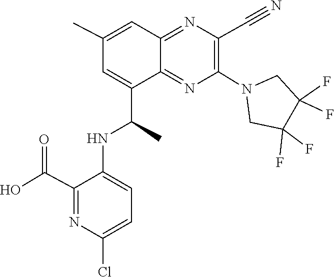 [SMILES: Cc1cc([C@@H](C)Nc2ccc(Cl)nc2C(=O)O)c2nc(N3CC(F)(F)C(F)(F)C3)c(C#N)nc2c1] |
| 745 | 6-chloro-3-(((1R)-1-(2-cyano-3-(6- hydroxy-2-azabicyclo[2.2.2]octan-2- yl)-7-methylquinoxalin-5- yl)ethyl)amino)picolinic acid | 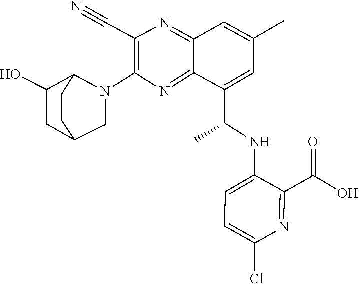 [SMILES: Cc1cc([C@@H](C)Nc2ccc(Cl)nc2C(=O)O)c2nc(N3CC4CCC3C(O)C4)c(C#N)nc2c1] |
| 746 | 6-chloro-3-(((1R)-1-(2-cyano-3-(4- fluoro-2-(morpholine-4- carbonyl)pyrrolidin-1-yl)-7- methylquinoxalin-5- yl)ethyl)amino)picolinic acid | 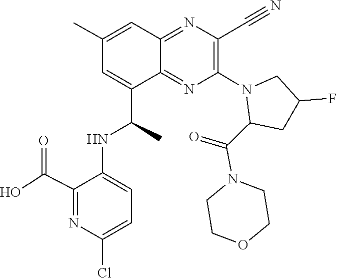 [SMILES: Cc1cc([C@@H](C)Nc2ccc(Cl)nc2C(=O)O)c2nc(N3CC(F)CC3C(=O)N3CCOCC3)c(C#N)nc2c1] |
| 747 | 6-chloro-3-(((1R)-1-(2-cyano-7- methyl-3-(1,2,3,4-tetrahydro-1,4- epiminonaphthalen-9-yl)quinoxalin-5- yl)ethyl)amino)picolinic acid | 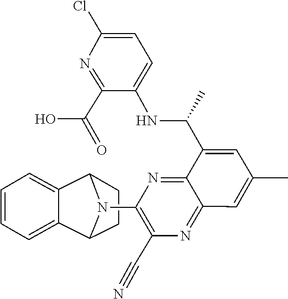 |
| 748 | (R)-6-chloro-3-((1-(2-cyano-3-(4,6- dihydro-5H-pyrrolo[3,4-d]thiazol-5-yl)- 7-methylquinoxalin-5- yl)ethyl)amino)picolinic acid | 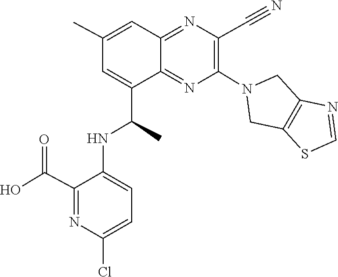 [SMILES: Cc1cc([C@@H](C)Nc2ccc(Cl)nc2C(=O)O)c2nc(N3Cc4ncsc4C3)c(C#N)nc2c1] |
| 749 | (R)-6-chloro-3-((1-(2-cyano-3-(5,7- dihydro-6H-pyrrolo[3,4-b]pyrazin-6- yl)-7-methylquinoxalin-5- yl)ethyl)amino)picolinic acid | 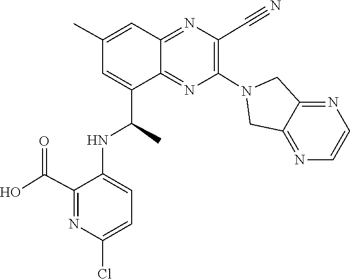 [SMILES: Cc1cc([C@@H](C)Nc2ccc(Cl)nc2C(=O)O)c2nc(N3Cc4nccnc4C3)c(C#N)nc2c1] |
| 750 | (R)-6-chloro-3-((1-(2-cyano-7-methyl- 3-(2-methyl-4,6-dihydro-5H- pyrrolo[3,4-d]thiazol-5-yl)quinoxalin- 5-yl)ethyl) amino)picolinic acid | 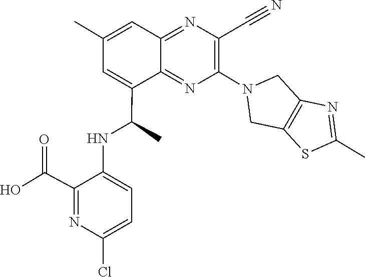 [SMILES: Cc1cc([C@@H](C)Nc2ccc(Cl)nc2C(=O)O)c2nc(N3Cc4nc(C)sc4C3)c(C#N)nc2c1] |
| 751 | (R)-6-chloro-3-((1-(2-cyano-3-(1,3- dihydro-2H-pyrrolo[3,4-c]pyridin-2-yl)- 7-methylquinoxalin-5- yl)ethyl)amino)picolinic acid | 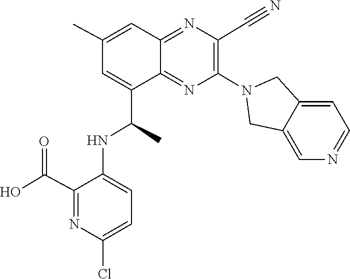 [SMILES: Cc1cc([C@@H](C)Nc2ccc(Cl)nc2C(=O)O)c2nc(N3Cc4ccncc4C3)c(C#N)nc2c1] |
| 752 | (R)-6-chloro-3-((1-(2-cyano-3-(5,7- dihydro-6H-pyrrolo[3,4-b]pyridin-6-yl)- 7-methylquinoxalin-5- yl)ethyl)amino)picolinic acid | 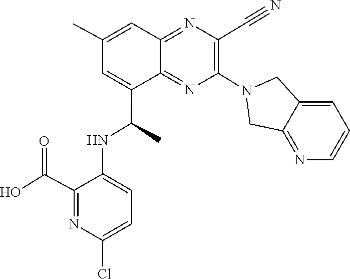 [SMILES: Cc1cc([C@@H](C)Nc2ccc(Cl)nc2C(=O)O)c2nc(N3Cc4cccnc4C3)c(C#N)nc2c1] |
| 753 | (R)-6-chloro-3-((1-(2-cyano-3-(4- fluoroisoindolin-2-yl)-7- methylquinoxalin-5- yl)ethyl)amino)picolinic acid | 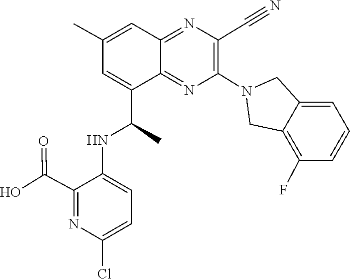 [SMILES: Cc1cc([C@@H](C)Nc2ccc(Cl)nc2C(=O)O)c2nc(N3Cc4cccc(F)c4C3)c(C#N)nc2c1] |
| 754 | (R)-6-chloro-3-((1-(2-cyano-3-(4- cyanoisoindolin-2-yl)-7- methylquinoxalin-5- yl)ethyl)amino)picolinic acid | 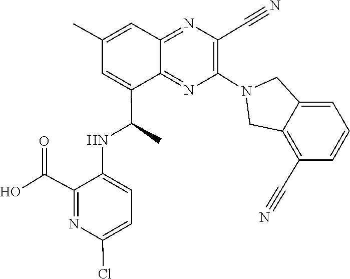 [SMILES: Cc1cc([C@@H](C)Nc2ccc(Cl)nc2C(=O)O)c2nc(N3Cc4cccc(C#N)c4C3)c(C#N)nc2c1] |
| 755 | (R)-6-chloro-3-((1-(2-cyano-3- (isoindolin-2-yl)-7-methylquinoxalin-5- yl)ethyl)amino)picolinic acid | 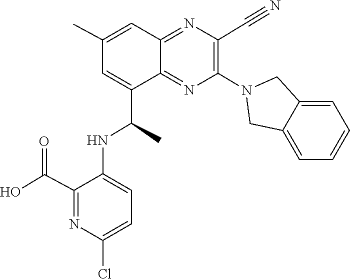 [SMILES: Cc1cc([C@@H](C)Nc2ccc(Cl)nc2C(=O)O)c2nc(N3Cc4ccccc4C3)c(C#N)nc2c1] |
| 756 | (R)-6-chloro-3-((1-(2-cyano-3-(5- (hydroxymethyl)isoindolin-2-yl)-7- methylquinoxalin-5- yl)ethyl)amino)picolinic acid | 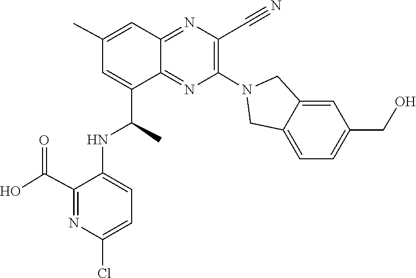 [SMILES: Cc1cc([C@@H](C)Nc2ccc(Cl)nc2C(=O)O)c2nc(N3Cc4ccc(CO)cc4C3)c(C#N)nc2c1] |
| 757 | (R)-6-chloro-3-((1-(2-cyano-7-methyl- 3-(5-methylisoindolin-2-yl)quinoxalin- 5-yl)ethyl)amino)picolinic acid | 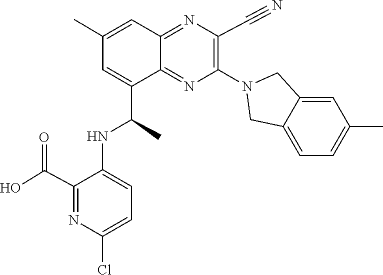 [SMILES: Cc1ccc2c(c1)CN(c1nc3c([C@@H](C)Nc4ccc(Cl)nc4C(=O)O)cc(C)cc3nc1C#N)C2] |
| 758 | (R)-6-chloro-3-((1-(2-cyano-3-(5- (difluoromethyl)isoindolin-2-yl)-7- methylquinoxalin-5- yl)ethyl)amino)picolinic acid | 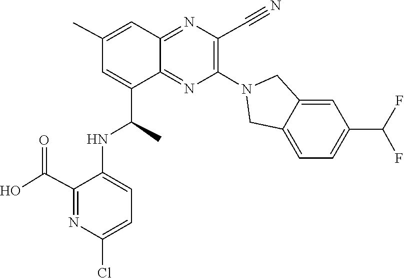 [SMILES: Cc1cc([C@@H](C)Nc2ccc(Cl)nc2C(=O)O)c2nc(N3Cc4ccc(C(F)F)cc4C3)c(C#N)nc2c1] |
| 759 | (R)-6-chloro-3-((1-(2-cyano-3-(5,6- dimethoxyisoindolin-2-yl)-7- methylquinoxalin-5- yl)ethyl)amino)picolinic acid | 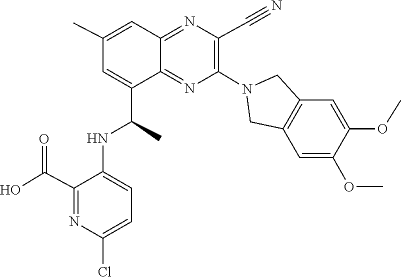 [SMILES: COc1cc2c(cc1OC)CN(c1nc3c([C@@H](C)Nc4ccc(Cl)nc4C(=O)O)cc(C)cc3nc1C#N)C2] |
| 760 | (R)-6-chloro-3-((1-(2-cyano-3-(5,6- difluoroisoindolin-2-yl)-7- methylquinoxalin-5- yl)ethyl)amino)picolinic acid | 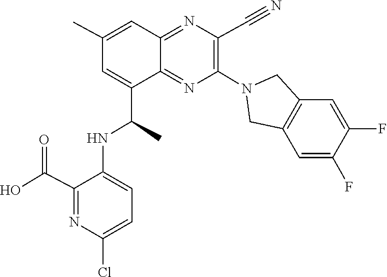 [SMILES: Cc1cc([C@@H](C)Nc2ccc(Cl)nc2C(=O)O)c2nc(N3Cc4cc(F)c(F)cc4C3)c(C#N)nc2c1] |
| 761 | 6-chloro-3-(((1R)-1-(2-cyano-7- methyl-3-(tetrahydro-1H,3H- spiro[pyrrolo[2, 1-c][1,4]oxazine-4,5′- [1,3]oxazinan]-3′-yl)quinoxalin-5- yl)ethyl)amino)picolinic acid | 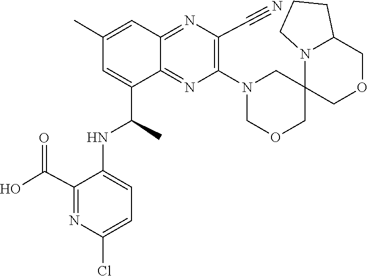 [SMILES: Cc1cc([C@@H](C)Nc2ccc(Cl)nc2C(=O)O)c2nc(N3COCC4(COCC5CCCN54)C3)c(C#N)nc2c1] |
| 762 | 6-chloro-3-(((1R)-1-(2-cyano-3- (decahydro-2H-cycloocta[c]pyrrol-2- yl)-7-methylquinoxalin-5- yl)ethyl)amino)picolinic acid | 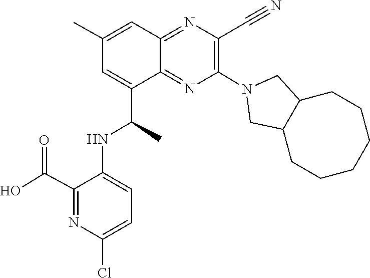 [SMILES: Cc1cc([C@@H](C)Nc2ccc(Cl)nc2C(=O)O)c2nc(N3CC4CCCCCCC4C3)c(C#N)nc2c1] |
| 763 | 6-chloro-3-(((1R)-1-(2-cyano-7- methyl-3-(8- azatricyclo[4.3.0.02,5]nonan-8- yl)quinoxalin-5- yl)ethyl)amino)picolinic acid | 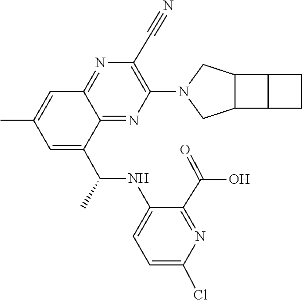 [SMILES: Cc1cc([C@@H](C)Nc2ccc(Cl)nc2C(=O)O)c2nc(N3CC4C5CCC5C4C3)c(C#N)nc2c1] |
| 764 | (R)-3-((1-(3-(1H,3H,4H-3a,6a- (methanooxymethano)furo[3,4- c]pyrrol-5(6H)-yl)-2-cyano-7- methylquinoxalin-5-yl)ethyl)amino)-6- chloropicolinic acid | 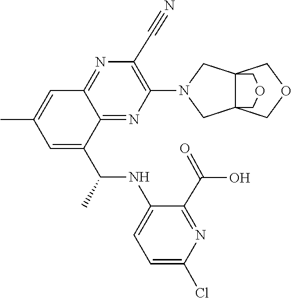 [SMILES: Cc1cc([C@@H](C)Nc2ccc(Cl)nc2C(=O)O)c2nc(N3CC45COCC4(COC5)C3)c(C#N)nc2c1] |
| 765 | 6-chloro-3-(((1R)-1-(2-cyano-3-(8- hydroxy-6-azaspiro[3.4]octan-6-yl)-7- methylquinoxalin-5- yl)ethyl)amino)picolinic acid | 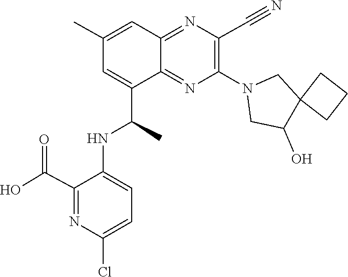 [SMILES: Cc1cc([C@@H](C)Nc2ccc(Cl)nc2C(=O)O)c2nc(N3CC(O)C4(CCC4)C3)c(C#N)nc2c1] |
| 766 | (R)-6-chloro-3-((1-(2-cyano-7-methyl- 3-(3-(5-(trifluoromethyl)isoxazol-3- yl)azetidin-1-yl)quinoxalin-5- yl)ethyl)amino)picolinic acid | 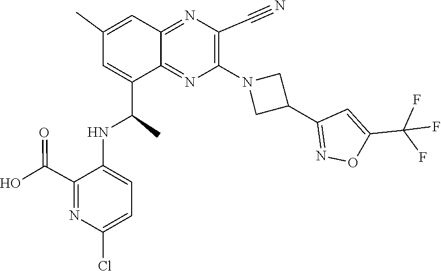 [SMILES: Cc1cc([C@@H](C)Nc2ccc(Cl)nc2C(=O)O)c2nc(N3CC(c4cc(C(F)(F)F)on4)C3)c(C#N)nc2c1] |
| 767 | (R)-6-chloro-3-((1-(2-cyano-7-methyl- 3-(3-(pyridin-2-yl)azetidin-1- yl)quinoxalin-5- yl)ethyl)amino)picolinic acid | 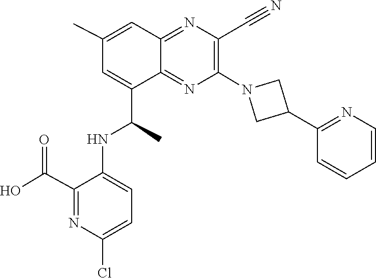 [SMILES: Cc1cc([C@@H](C)Nc2ccc(Cl)nc2C(=O)O)c2nc(N3CC(c4ccccn4)C3)c(C#N)nc2c1] |
| 768 | (R)-6-chloro-3-((1-(2-cyano-7-methyl- 3-(3-(pyridin-3-yl)azetidin-1- yl)quinoxalin-5- yl)ethyl)amino)picolinic acid | 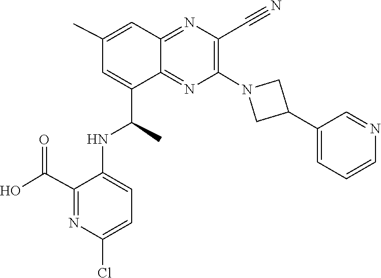 [SMILES: Cc1cc([C@@H](C)Nc2ccc(Cl)nc2C(=O)O)c2nc(N3CC(c4cccnc4)C3)c(C#N)nc2c1] |
| 769 | (R)-6-chloro-3-((1-(2-cyano-7-methyl- 3-(3-phenylazetidin-1-yl)quinoxalin-5- yl)ethyl)amino)picolinic acid | 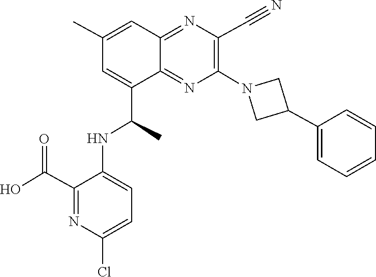 [SMILES: Cc1cc([C@@H](C)Nc2ccc(Cl)nc2C(=O)O)c2nc(N3CC(c4ccccc4)C3)c(C#N)nc2c1] |
| 770 | (R)-6-chloro-3-((1-(2-cyano-3-(3- methoxyazetidin-1-yl)-7- methylquinoxalin-5- yl)ethyl)amino)picolinic acid | 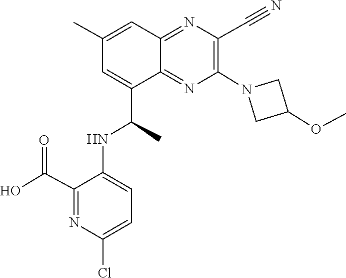 [SMILES: COC1CN(c2nc3c([C@@H](C)Nc4ccc(Cl)nc4C(=O)O)cc(C)cc3nc2C#N)C1] |
| 771 | (R)-6-chloro-3-((1-(2-cyano-3-(3- fluoroazetidin-1-yl)-7- methylquinoxalin-5- yl)ethyl)amino)picolinic acid | 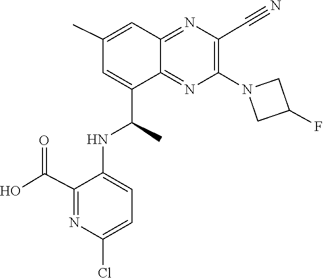 [SMILES: Cc1cc([C@@H](C)Nc2ccc(Cl)nc2C(=O)O)c2nc(N3CC(F)C3)c(C#N)nc2c1] |
| 772 | (R)-6-chloro-3-((1-(2-cyano-3-(3- (difluoromethyl)azetidin-1-yl)-7- methylquinoxalin-5- yl)ethyl)amino)picolinic acid | 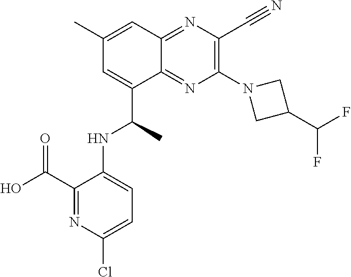 [SMILES: Cc1cc([C@@H](C)Nc2ccc(Cl)nc2C(=O)O)c2nc(N3CC(C(F)F)C3)c(C#N)nc2c1] |
| 773 | (R)-6-chloro-3-((1-(2-cyano-3-(3- cyanoazetidin-1-yl)-7- methylquinoxalin-5- yl)ethyl)amino)picolinic acid | 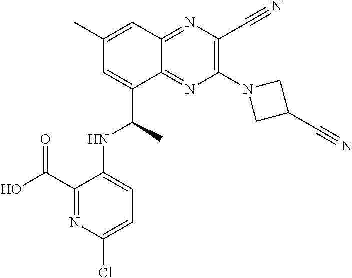 [SMILES: Cc1cc([C@@H](C)Nc2ccc(Cl)nc2C(=O)O)c2nc(N3CC(C#N)C3)c(C#N)nc2c1] |
| 774 | (R)-6-chloro-3-((1-(2-cyano-3-(7-(4- cyanophenyl)-2,7- diazaspiro[3.5]nonan-2-yl)-7- methylquinoxalin-5- yl)ethyl)amino)picolinic acid | 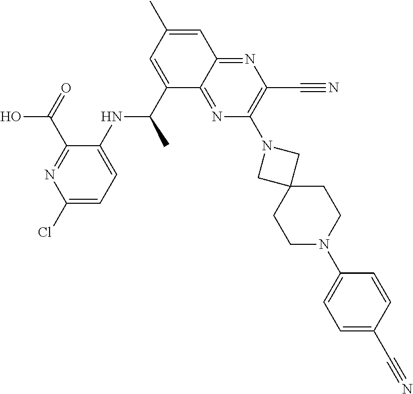 [SMILES: Cc1cc([C@@H](C)Nc2ccc(Cl)nc2C(=O)O)c2nc(N3CC4(CCN(c5ccc(C#N)cc5)CC4)C3)c(C#N)nc2c1] |
| 775 | 6-chloro-3-(((1R)-1-(2-cyano-3-(8- hydroxyoctahydro-2H-4,7- methanoisoindol-2-yl)-7- methylquinoxalin-5- yl)ethyl)amino)picolinic acid | 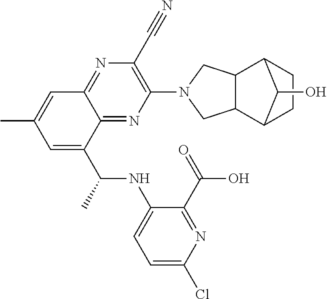 [SMILES: Cc1cc([C@@H](C)Nc2ccc(Cl)nc2C(=O)O)c2nc(N3CC4C5CCC(C5O)C4C3)c(C#N)nc2c1] |
| 776 | 6-chloro-3-(((1R)-1-(2-cyano-7- methyl-3-(5-methyl-2- azabicyclo[2.2.1]heptan-2- yl)quinoxalin-5- yl)ethyl)amino)picolinic acid | 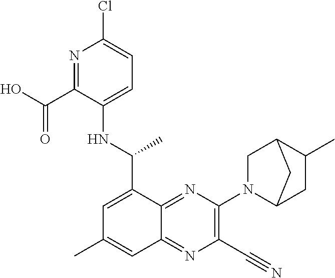 [SMILES: Cc1cc([C@@H](C)Nc2ccc(Cl)nc2C(=O)O)c2nc(N3CC4CC3CC4C)c(C#N)nc2c1] |
| 777 | 6-chloro-3-(((1R)-1-(2-cyano-7- methyl-3-(1′- methyltetrahydrospiro[furo[3,4- b]pyrrole-3,4′-piperidin]-1(2H)- yl)quinoxalin-5- yl)ethyl)amino)picolinic acid | 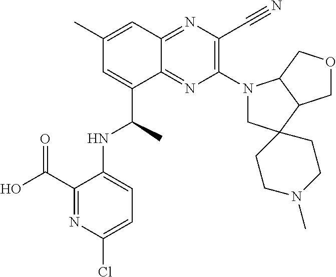 [SMILES: Cc1cc([C@@H](C)Nc2ccc(Cl)nc2C(=O)O)c2nc(N3CC4(CCN(C)CC4)C4COCC43)c(C#N)nc2c1] |
| 778 | 6-chloro-3-(((1R)-1-(2-cyano-3-(1- (hydroxymethyl)isoindolin-2-yl)-7- methylquinoxalin-5- yl)ethyl)amino)picolinic acid | 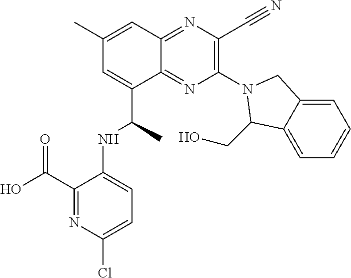 [SMILES: Cc1cc([C@@H](C)Nc2ccc(Cl)nc2C(=O)O)c2nc(N3Cc4ccccc4C3CO)c(C#N)nc2c1] |
| 779 | 6-chloro-3-(((1R)-1-(3-(7-chloro-3- (hydroxymethyl)-3,4- dihydroisoquinolin-2(1H)-yl)-2-cyano- 7-methylquinoxalin-5- yl)ethyl)amino)picolinic acid | 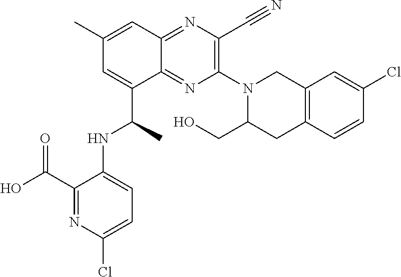 [SMILES: Cc1cc([C@@H](C)Nc2ccc(Cl)nc2C(=O)O)c2nc(N3Cc4cc(Cl)ccc4CC3CO)c(C#N)nc2c1] |
| 780 | 6-chloro-3-(((1R)-1-(2-cyano-3-(3- hydroxyoctahydro-1H-indol-1-yl)-7- methylquinoxalin-5- yl)ethyl)amino)picolinic acid | 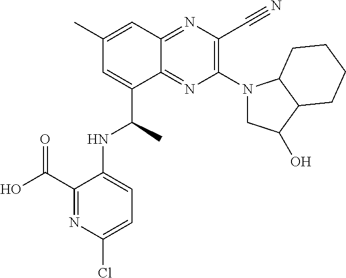 [SMILES: Cc1cc([C@@H](C)Nc2ccc(Cl)nc2C(=O)O)c2nc(N3CC(O)C4CCCCC43)c(C#N)nc2c1] |
| 781 | 6-chloro-3-(((1R)-1-(2-cyano-7- methyl-3-(1-methyloctahydro-2H- isoindol-2-yl)quinoxalin-5- yl)ethyl)amino)picolinic acid | 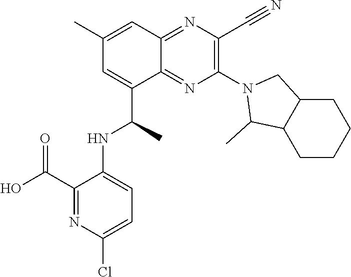 [SMILES: Cc1cc([C@@H](C)Nc2ccc(Cl)nc2C(=O)O)c2nc(N3CC4CCCCC4C3C)c(C#N)nc2c1] |
| 782 | 6-chloro-3-(((1R)-1-(2-cyano-3-(3,3- difluoro-2-methylazetidin-1-yl)-7- methylquinoxalin-5- yl)ethyl)amino)picolinic acid | 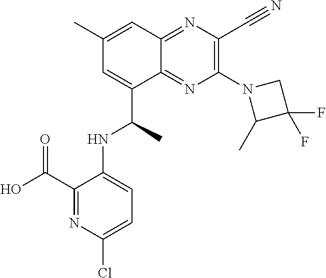 [SMILES: Cc1cc([C@@H](C)Nc2ccc(Cl)nc2C(=O)O)c2nc(N3CC(F)(F)C3C)c(C#N)nc2c1] |
| 783 | (R)-6-chloro-3-((1-(2-cyano-7-methyl- 3-(methyl((5,6,8,9-tetrahydro- [1,2,4]triazolo[4,3-d][1,4]oxazepin-3- yl)methyl)amino)quinoxalin-5- yl)ethyl)amino)picolinic acid | 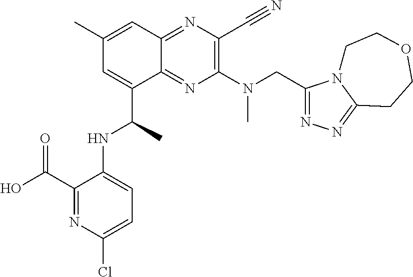 [SMILES: Cc1cc([C@@H](C)Nc2ccc(Cl)nc2C(=O)O)c2nc(N(C)Cc3nnc4n3CCOCC4)c(C#N)nc2c1] |
| 784 | 6-chloro-3-(((1R)-1-(2-cyano-7- methyl-3-(methyl((5,6,7,8- tetrahydroimidazo[1,2-a]pyridin-7- yl)methyl)amino)quinoxalin-5- yl)ethyl)amino)picolinic acid | 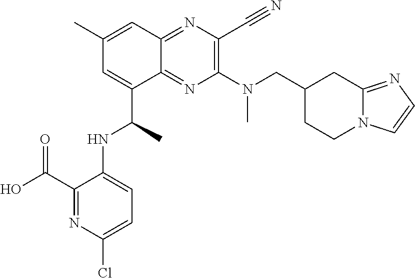 [SMILES: Cc1cc([C@@H](C)Nc2ccc(Cl)nc2C(=O)O)c2nc(N(C)CC3CCn4ccnc4C3)c(C#N)nc2c1] |
| 785 | (R)-6-chloro-3-((1-(2-cyano-3- ((cyclopropylmethyl)(methyl)amino)- 7-methylquinoxalin-5- yl)ethyl)amino)picolinic acid | 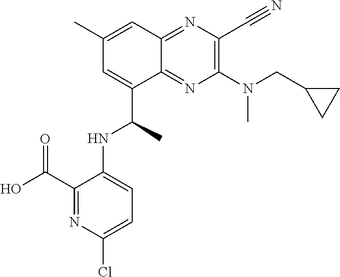 [SMILES: Cc1cc([C@@H](C)Nc2ccc(Cl)nc2C(=O)O)c2nc(N(C)CC3CC3)c(C#N)nc2c1] |
| 786 | 6-chloro-3-(((1R)-1-(2-cyano-3-(((1- hydroxy-2,3-dihydro-1H-inden-1- yl)methyl)(methyl)amino)-7- methylquinoxalin-5- yl)ethyl)amino)picolinic acid | 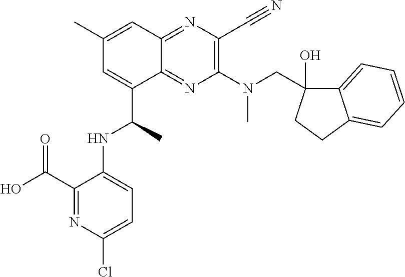 [SMILES: Cc1cc([C@@H](C)Nc2ccc(Cl)nc2C(=O)O)c2nc(N(C)CC3(O)CCc4ccccc43)c(C#N)nc2c1] |
| 787 | (R)-6-chloro-3-((1-(2-cyano-3- (dimethylamino)-7-methylquinoxalin- 5-yl)ethyl)amino)picolinic acid | 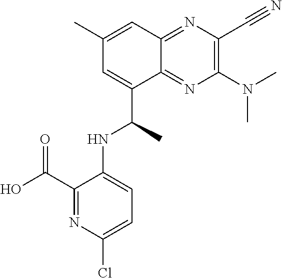 [SMILES: Cc1cc([C@@H](C)Nc2ccc(Cl)nc2C(=O)O)c2nc(N(C)C)c(C#N)nc2c1] |
| 788 | 3-(((1R)-1-(3-((1-benzyl-2- oxopiperidin-3-yl)(methyl)amino)-2- cyano-7-methylquinoxalin-5- yl)ethyl)amino)-6-chloropicolinic acid | 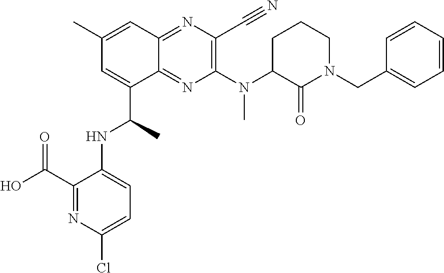 [SMILES: Cc1cc([C@@H](C)Nc2ccc(Cl)nc2C(=O)O)c2nc(N(C)C3CCCN(Cc4ccccc4)C3=O)c(C#N)nc2c1] |
| 789 | 6-chloro-3-(((1R)-1-(2-cyano-7- methyl-3-(methyl(4,5,6,7- tetrahydrobenzo[d]thiazol-6- yl)amino)quinoxalin-5- yl)ethyl)amino)picolinic acid | 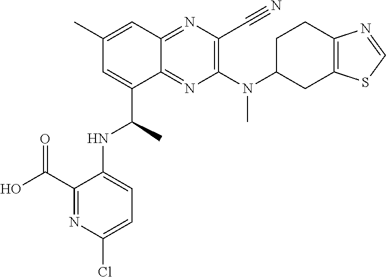 [SMILES: Cc1cc([C@@H](C)Nc2ccc(Cl)nc2C(=O)O)c2nc(N(C)C3CCc4ncsc4C3)c(C#N)nc2c1] |
| 790 | (R)-6-chloro-3-((1-(2-cyano-3-((3,3- difluorocyclobutyl)(methyl)amino)-7- methylquinoxalin-5- yl)ethyl)amino)picolinic acid | 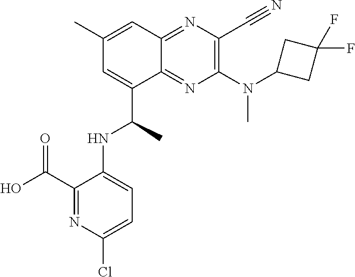 [SMILES: Cc1cc([C@@H](C)Nc2ccc(Cl)nc2C(=O)O)c2nc(N(C)C3CC(F)(F)C3)c(C#N)nc2c1] |
| 791 | 6-chloro-3-(((1R)-1-(2-cyano-7- methyl-3-(methyl(1-methyl-2,3- dihydro-1H-inden-2- yl)amino)quinoxalin-5- yl)ethyl)amino)picolinic acid | 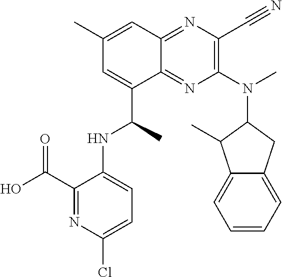 [SMILES: Cc1cc([C@@H](C)Nc2ccc(Cl)nc2C(=O)O)c2nc(N(C)C3Cc4ccccc4C3C)c(C#N)nc2c1] |
| 792 | 6-chloro-3-(((1R)-1-(2-cyano-3-((2- methoxy-2,3-dihydro-1H-inden-1- yl)(methyl)amino)-7- methylquinoxalin-5- yl)ethyl)amino)picolinic acid | 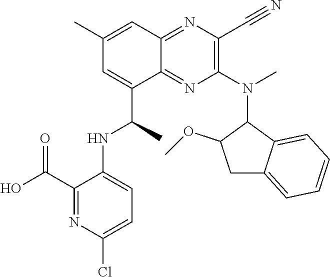 [SMILES: COC1Cc2ccccc2C1N(C)c1nc2c([C@@H](C)Nc3ccc(Cl)nc3C(=O)O)cc(C)cc2nc1C#N] |
| 793 | 6-chloro-3-(((1R)-1-(2-cyano-3-(3-(4- cyanophenyl)-3,8- diazabicyclo[3.2.1]octan-8-yl)-7- methylquinoxalin-5- yl)ethyl)amino)picolinic acid | 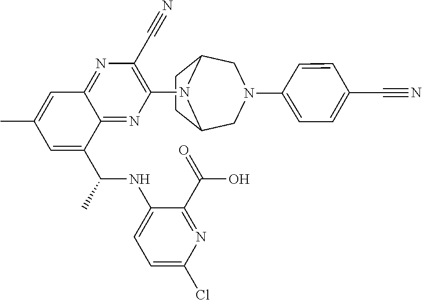 [SMILES: Cc1cc([C@@H](C)Nc2ccc(Cl)nc2C(=O)O)c2nc(N3C4CCC3CN(c3ccc(C#N)cc3)C4)c(C#N)nc2c1] |
| 794 | 6-chloro-3-(((1R)-1-(2-cyano-7- methyl-3-(3-methyl-4-oxo-3,9- diazabicyclo[4.2.1]nonan-9- yl)quinoxalin-5- yl)ethyl)amino)picolinic acid | 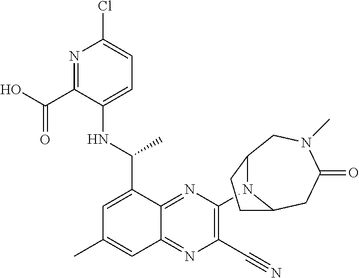 [SMILES: Cc1cc([C@@H](C)Nc2ccc(Cl)nc2C(=O)O)c2nc(N3C4CCC3CN(C)C(=O)C4)c(C#N)nc2c1] |
| 795 | 6-chloro-3-(((1R)-1-(2-cyano-3-(3,3- difluoro-8-azabicyclo[3.2.1]octan-8- yl)-7-methylquinoxalin-5- yl)ethyl)amino)picolinic acid | 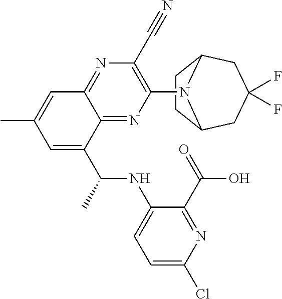 [SMILES: Cc1cc([C@@H](C)Nc2ccc(Cl)nc2C(=O)O)c2nc(N3C4CCC3CC(F)(F)C4)c(C#N)nc2c1] |
| 796 | 6-chloro-3-(((1R)-1-(2-cyano-3-(3-(4- cyanophenyl)-3,6- diazabicyclo[3.1.1]heptan-6-yl)-7- methylquinoxalin-5- yl)ethyl)amino)picolinic acid | 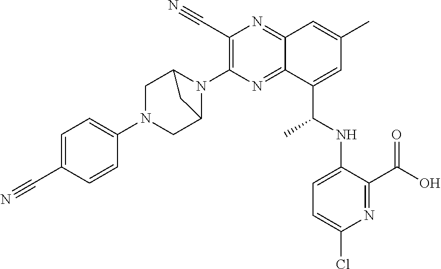 [SMILES: Cc1cc([C@@H](C)Nc2ccc(Cl)nc2C(=O)O)c2nc(N3C4CC3CN(c3ccc(C#N)cc3)C4)c(C#N)nc2c1] |
| 797 | (R)-6-chloro-3-((1-(2-cyano-7-methyl- 3-(piperidin-4-yl)quinoxalin-5- yl)ethyl)amino)picolinic acid | 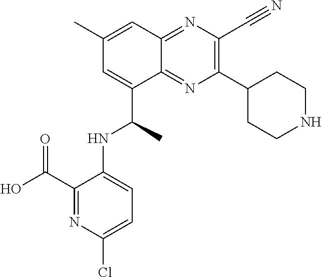 [SMILES: Cc1cc([C@@H](C)Nc2ccc(Cl)nc2C(=O)O)c2nc(C3CCNCC3)c(C#N)nc2c1] |
| 798 | (R)-6-chloro-3-((1-(2-cyano-7-methyl- 3-(1-methylpiperidin-4-yl)quinoxalin- 5-yl)ethyl)amino)picolinic acid | 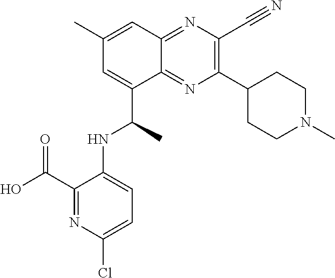 [SMILES: Cc1cc([C@@H](C)Nc2ccc(Cl)nc2C(=O)O)c2nc(C3CCN(C)CC3)c(C#N)nc2c1] |
| 799 | (R)-6-chloro-3-((1-(2-cyano-3-(3,6- dihydro-2H-pyran-4-yl)-7- methylquinoxalin-5- yl)ethyl)amino)picolinic acid | 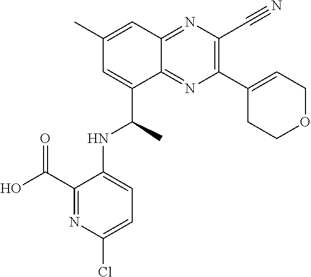 [SMILES: Cc1cc([C@@H](C)Nc2ccc(Cl)nc2C(=O)O)c2nc(C3=CCOCC3)c(C#N)nc2c1] |
| 800 | (R)-6-chloro-3-((1-(2-cyano-7-methyl- 3-(1,2,3,6-tetrahydropyridin-4- yl)quinoxalin-5- yl)ethyl)amino)picolinic acid | 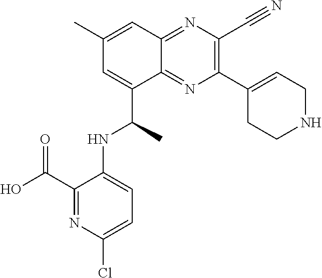 [SMILES: Cc1cc([C@@H](C)Nc2ccc(Cl)nc2C(=O)O)c2nc(C3=CCNCC3)c(C#N)nc2c1] |
| 801 | (R)-6-chloro-3-((1-(2-cyano-7-methyl- 3-(1-methyl-1,2,3,6-tetrahydropyridin- 4-yl)quinoxalin-5- yl)ethyl)amino)picolinic acid | 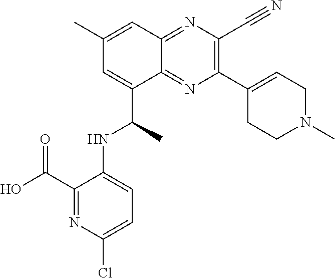 [SMILES: Cc1cc([C@@H](C)Nc2ccc(Cl)nc2C(=O)O)c2nc(C3=CCN(C)CC3)c(C#N)nc2c1] |
| 802 | (R)-6-chloro-3-((1-(2-cyano-3,7- dimethylquinoxalin-5- yl)ethyl)amino)picolinic acid | 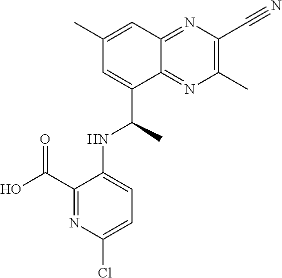 [SMILES: Cc1cc([C@@H](C)Nc2ccc(Cl)nc2C(=O)O)c2nc(C)c(C#N)nc2c1] |
| 803 | 6-chloro-3-(((1R)-1-(2-cyano-7- methyl-3-(1-methyl-2-oxopiperidin-4- yl)quinoxalin-5- yl)ethyl)amino)picolinic acid | 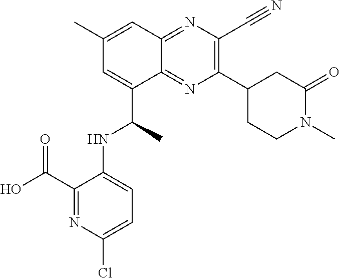 [SMILES: Cc1cc([C@@H](C)Nc2ccc(Cl)nc2C(=O)O)c2nc(C3CCN(C)C(=O)C3)c(C#N)nc2c1] |
| 804 | (R)-6-chloro-3-((1-(2-cyano-3-(1-(4- cyanophenyl)piperidin-4-yl)-7- methylquinoxalin-5- yl)ethyl)amino)picolinic acid | 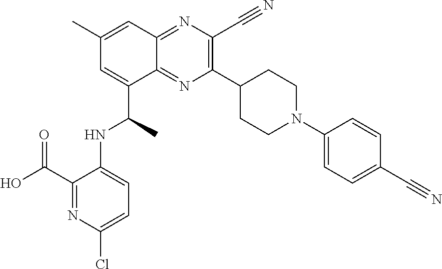 [SMILES: Cc1cc([C@@H](C)Nc2ccc(Cl)nc2C(=O)O)c2nc(C3CCN(c4ccc(C#N)cc4)CC3)c(C#N)nc2c1] |
| 805 | (R)-3-((1-(3-(1-acetylpiperidin-4-yl)-2- cyano-7-methylquinoxalin-5- yl)ethyl)amino)-6-chloropicolinic acid | 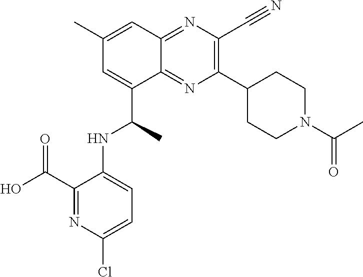 [SMILES: CC(=O)N1CCC(c2nc3c([C@@H](C)Nc4ccc(Cl)nc4C(=O)O)cc(C)cc3nc2C#N)CC1] |
| 806 | (R)-6-chloro-3-((1-(2-cyano-3-(4,4- difluorocyclohexyl)-7- methylquinoxalin-5- yl)ethyl)amino)picolinic acid | 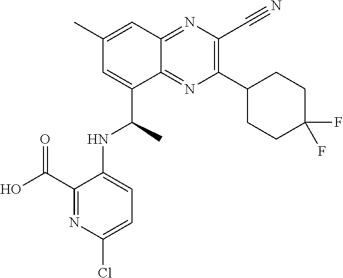 [SMILES: Cc1cc([C@@H](C)Nc2ccc(Cl)nc2C(=O)O)c2nc(C3CCC(F)(F)CC3)c(C#N)nc2c1] |
| 807 | (R)-3-((1-(3-(1-acetyl-1,2,3,6- tetrahydropyridin-4-yl)-2-cyano-7- methylquinoxalin-5-yl)ethyl)amino)-6- chloropicolinic acid | 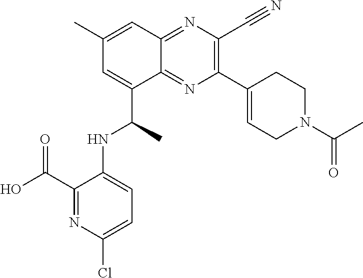 [SMILES: CC(=O)N1CC=C(c2nc3c([C@@H](C)Nc4ccc(Cl)nc4C(=O)O)cc(C)cc3nc2C#N)CC1] |
| 808 | (R)-6-chloro-3-((1-(2-cyano-3-((4- cyanophenyl)ethynyl)-7- methylquinoxalin-5- yl)ethyl)amino)picolinic acid | 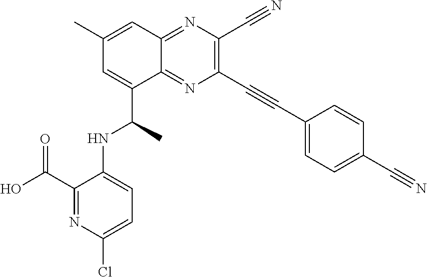 [SMILES: Cc1cc([C@@H](C)Nc2ccc(Cl)nc2C(=O)O)c2nc(C#Cc3ccc(C#N)cc3)c(C#N)nc2c1] |
| 809 | (R)-6-chloro-3-((1-(2-cyano-7-methyl- 3-(1-methyl-1H-pyrazol-3- yl)quinoxalin-5- yl)ethyl)amino)picolinic acid | 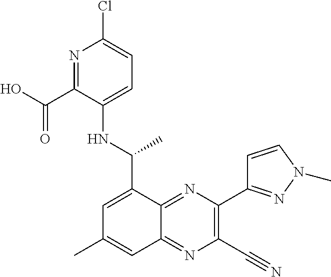 [SMILES: Cc1cc([C@@H](C)Nc2ccc(Cl)nc2C(=O)O)c2nc(-c3ccn(C)n3)c(C#N)nc2c1] |
| 810 | (R)-6-chloro-3-((1-(2-cyano-7-methyl- 3-(pyridin-2-yl)quinoxalin-5- yl)ethyl)amino)picolinic acid | 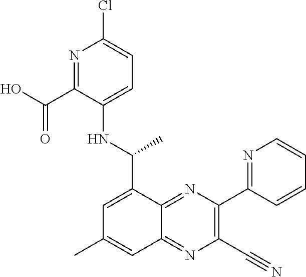 [SMILES: Cc1cc([C@@H](C)Nc2ccc(Cl)nc2C(=O)O)c2nc(-c3ccccn3)c(C#N)nc2c1] |
| 811 | (R)-6-chloro-3-((1-(2-cyano-7-methyl- 3-(pyridin-3-yl)quinoxalin-5- yl)ethyl)amino)picolinic acid | 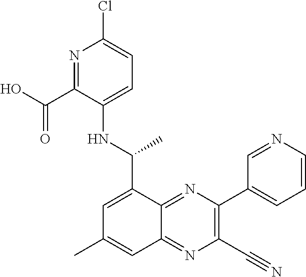 [SMILES: Cc1cc([C@@H](C)Nc2ccc(Cl)nc2C(=O)O)c2nc(-c3cccnc3)c(C#N)nc2c1] |
| 812 | (R)-6-chloro-3-((1-(2-cyano-7-methyl- 3-(1-methyl-1H-pyrazol-4- yl)quinoxalin-5- yl)ethyl)amino)picolinic acid | 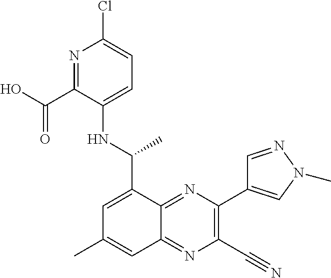 |
| 813 | (R)-6-chloro-3-((1-(2-cyano-7-methyl- 3-(1-methyl-1H-imidazol-4- yl)quinoxalin-5- yl)ethyl)amino)picolinic acid | 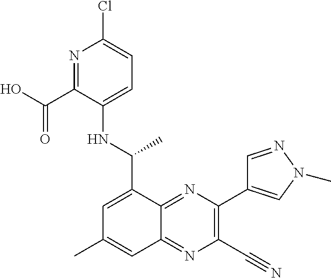 |
| 814 | (R)-6-chloro-3-((1-(2-cyano-3-(1-(4- cyanophenyl)-1H-pyrazol-4-yl)-7- methylquinoxalin-5- yl)ethyl)amino)picolinic acid | 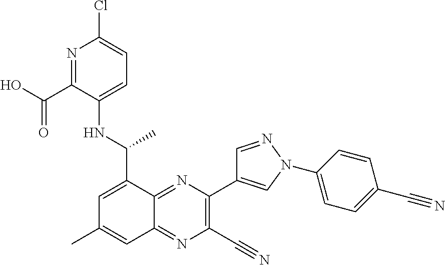 |
| 815 | (R)-6-chloro-3-((1-(2-cyano-7-methyl- 3-(pyridin-4-yl)quinoxalin-5- yl)ethyl)amino)picolinic acid | 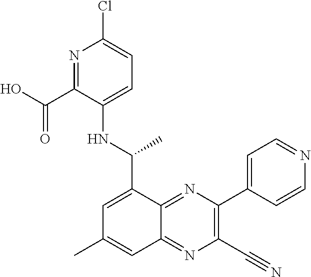 [SMILES: Cc1cc([C@@H](C)Nc2ccc(Cl)nc2C(=O)O)c2nc(-c3ccncc3)c(C#N)nc2c1] |
| 816 | (R)-6-chloro-3-((1-(2-cyano-7-methyl- 3-phenylquinoxalin-5- yl)ethyl)amino)picolinic acid | 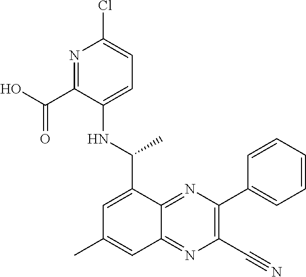 [SMILES: Cc1cc([C@@H](C)Nc2ccc(Cl)nc2C(=O)O)c2nc(-c3ccccc3)c(C#N)nc2c1] |
| 817 | (R)-6-chloro-3-((1-(2-cyano-7-methyl- 3-(p-tolyl)quinoxalin-5- yl)ethyl)amino)picolinic acid | 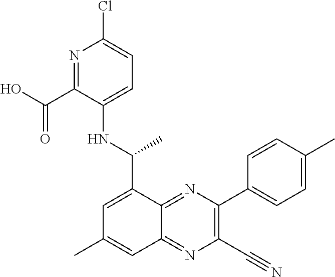 [SMILES: Cc1ccc(-c2nc3c([C@@H](C)Nc4ccc(Cl)nc4C(=O)O)cc(C)cc3nc2C#N)cc1] |
| 818 | (R)-6-chloro-3-((1-(2-cyano-3-(3- methoxyphenyl)-7-methylquinoxalin- 5-yl)ethyl)amino)picolinic acid | 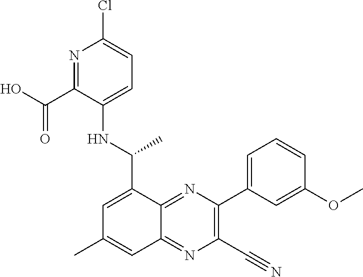 [SMILES: COc1cccc(-c2nc3c([C@@H](C)Nc4ccc(Cl)nc4C(=O)O)cc(C)cc3nc2C#N)c1] |
| 819 | (R)-6-chloro-3-((1-(2-cyano-3-(3-(2- cyanopropan-2-yl)phenyl)-7- methylquinoxalin-5- yl)ethyl)amino)picolinic acid | 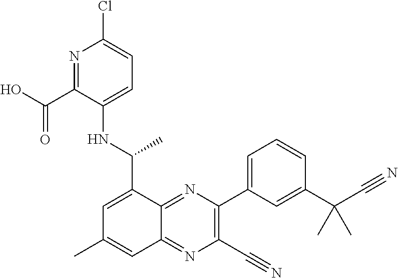 [SMILES: Cc1cc([C@@H](C)Nc2ccc(Cl)nc2C(=O)O)c2nc(-c3cccc(C(C)(C)C#N)c3)c(C#N)nc2c1] |
| 820 | (R)-6-chloro-3-((1-(2-cyano-3-(3- cyanophenyl)-7-methylquinoxalin-5- yl)ethyl)amino)picolinic acid | 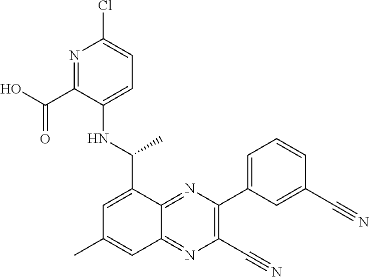 [SMILES: Cc1cc([C@@H](C)Nc2ccc(Cl)nc2C(=O)O)c2nc(-c3cccc(C#N)c3)c(C#N)nc2c1] |
| 821 | (R)-6-chloro-3-((1-(2-cyano-7-methyl- 3-(1H-pyrazol-4-yl)quinoxalin-5- yl)ethyl)amino)picolinic acid | 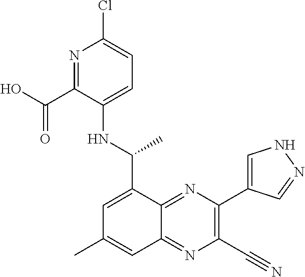 [SMILES: Cc1cc([C@@H](C)Nc2ccc(Cl)nc2C(=O)O)c2nc(-c3cn[nH]c3)c(C#N)nc2c1] |
| 822 | (R)-6-chloro-3-((1-(2-cyano-3-(1H- imidazol-4-yl)-7-methylquinoxalin-5- yl)ethyl)amino)picolinic acid | 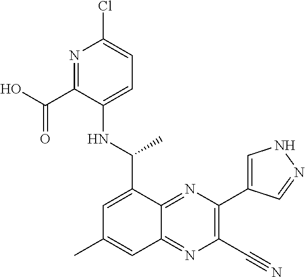 [SMILES: Cc1cc([C@@H](C)Nc2ccc(Cl)nc2C(=O)O)c2nc(-c3cn[nH]c3)c(C#N)nc2c1] |
| 823 | (R)-6-chloro-3-((1-(2-cyano-7-methyl- 3-(o-tolyl)quinoxalin-5- yl)ethyl)amino)picolinic acid | 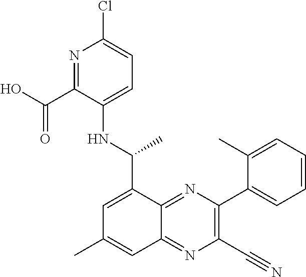 [SMILES: Cc1cc([C@@H](C)Nc2ccc(Cl)nc2C(=O)O)c2nc(-c3ccccc3C)c(C#N)nc2c1] |
| 824 | (R)-6-chloro-3-((1-(2-cyano-3-(1-(4- cyanophenyl)-1H-imidazol-4-yl)-7- methylquinoxalin-5- yl)ethyl)amino)picolinic acid | 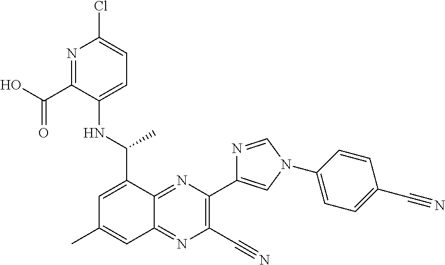 [SMILES: Cc1cc([C@@H](C)Nc2ccc(Cl)nc2C(=O)O)c2nc(-c3cn(-c4ccc(C#N)cc4)cn3)c(C#N)nc2c1] |
| 825 | (R)-6-chloro-3-((1-(2-cyano-3-(2- methoxyphenyl)-7-methylquinoxalin- 5-yl)ethyl)amino)picolinic acid | 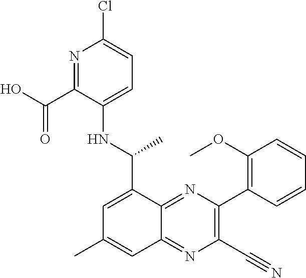 [SMILES: COc1ccccc1-c1nc2c([C@@H](C)Nc3ccc(Cl)nc3C(=O)O)cc(C)cc2nc1C#N] |
| 826 | (R)-6-chloro-3-((1-(2-cyano-3-(2- cyanophenyl)-7-methylquinoxalin-5- yl)ethyl)amino)picolinic acid | 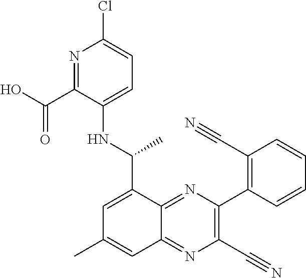 [SMILES: Cc1cc([C@@H](C)Nc2ccc(Cl)nc2C(=O)O)c2nc(-c3ccccc3C#N)c(C#N)nc2c1] |
| 827 | (R)-6-chloro-3-((1-(2-cyano-3-(4- methoxyphenyl)-7-methylquinoxalin- 5-yl)ethyl)amino)picolinic acid | 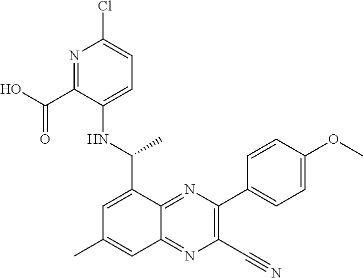 [SMILES: COc1ccc(-c2nc3c([C@@H](C)Nc4ccc(Cl)nc4C(=O)O)cc(C)cc3nc2C#N)cc1] |
| 828 | (R)-6-chloro-3-((1-(2-cyano-3-(4- fluorophenyl)-7-methylquinoxalin-5- yl)ethyl)amino)picolinic acid | 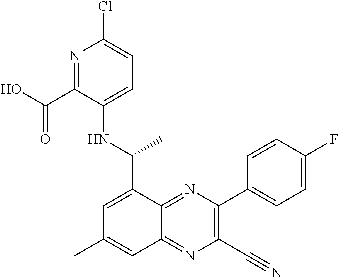 [SMILES: Cc1cc([C@@H](C)Nc2ccc(Cl)nc2C(=O)O)c2nc(-c3ccc(F)cc3)c(C#N)nc2c1] |
| 829 | (R)-6-chloro-3-((1-(3-(4- chlorophenyl)-2-cyano-7- methylquinoxalin-5- yl)ethyl)amino)picolinic acid | 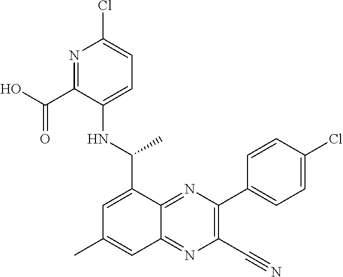 [SMILES: Cc1cc([C@@H](C)Nc2ccc(Cl)nc2C(=O)O)c2nc(-c3ccc(Cl)cc3)c(C#N)nc2c1] |
| 830 | (R)-6-chloro-3-((1-(2-cyano-3-(4- cyanophenyl)-7-methylquinoxalin-5- yl)ethyl)amino)picolinic acid | 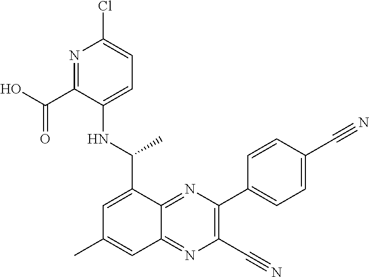 [SMILES: Cc1cc([C@@H](C)Nc2ccc(Cl)nc2C(=O)O)c2nc(-c3ccc(C#N)cc3)c(C#N)nc2c1] |
| 831 | (R)-6-chloro-3-((1-(3-chloro-2-cyano- 7-methylquinoxalin-5- yl)ethyl)amino)picolinic acid | 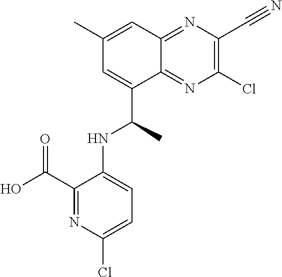 [SMILES: Cc1cc([C@@H](C)Nc2ccc(Cl)nc2C(=O)O)c2nc(Cl)c(C#N)nc2c1] |
| 832 | 6-chloro-3-(((1R)-1-(2-cyano-7- methyl-3-((tetrahydrofuran-3- yl)thio)quinoxalin-5- yl)ethyl)amino)picolinic acid | 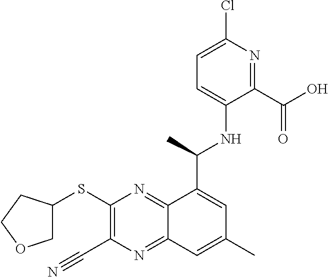 [SMILES: Cc1cc([C@@H](C)Nc2ccc(Cl)nc2C(=O)O)c2nc(SC3CCOC3)c(C#N)nc2c1] |
| 833 | (R)-6-chloro-3-((1-(2-cyano-7-methyl- 3-phenoxyquinoxalin-5- yl)ethyl)amino)picolinic acid | 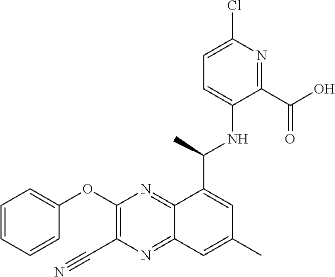 [SMILES: Cc1cc([C@@H](C)Nc2ccc(Cl)nc2C(=O)O)c2nc(Oc3ccccc3)c(C#N)nc2c1] |
| 834 | (R)-6-chloro-3-((1-(2-cyano-3-(3- cyanophenoxy)-7-methylquinoxalin-5- yl)ethyl)amino)picolinic acid | 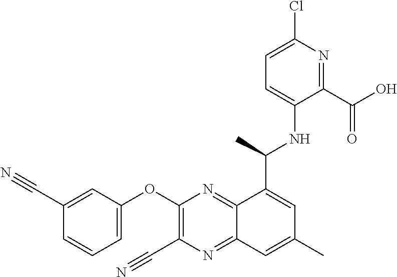 [SMILES: Cc1cc([C@@H](C)Nc2ccc(Cl)nc2C(=O)O)c2nc(Oc3cccc(C#N)c3)c(C#N)nc2c1] |
| 835 | (R)-6-chloro-3-((1-(2-cyano-7-methyl- 3- (((trifluoromethyl)sulfonyl)oxy)quinox- alin-5-yl)ethyl)amino)picolinic acid | 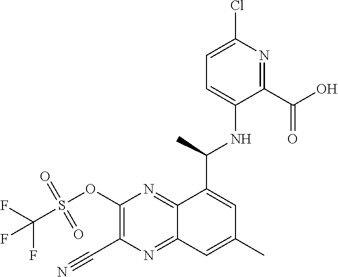 [SMILES: Cc1cc([C@@H](C)Nc2ccc(Cl)nc2C(=O)O)c2nc(OS(=O)(=O)C(F)(F)F)c(C#N)nc2c1] |
| 836 | (R)-3-((1-(3-(benzyloxy)-2-cyano-7- methylquinoxalin-5-yl)ethyl)amino)-6- chloropicolinic acid | 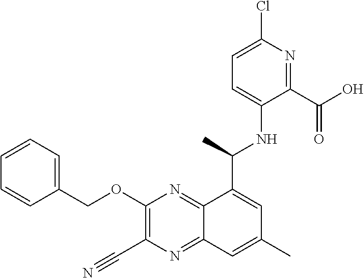 [SMILES: Cc1cc([C@@H](C)Nc2ccc(Cl)nc2C(=O)O)c2nc(OCc3ccccc3)c(C#N)nc2c1] |
| 837 | 6-chloro-3-(((1R)-1-(2-cyano-7- methyl-3-((tetrahydrofuran-3- yl)oxy)quinoxalin-5- yl)ethyl)amino)picolinic acid | 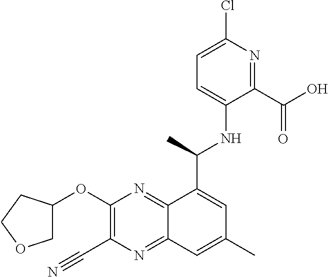 [SMILES: Cc1cc([C@@H](C)Nc2ccc(Cl)nc2C(=O)O)c2nc(OC3CCOC3)c(C#N)nc2c1] |
| 838 | (R)-6-chloro-3-((1-(2-cyano-3- methoxy-7-methylquinoxalin-5- yl)ethyl)amino)picolinic acid | 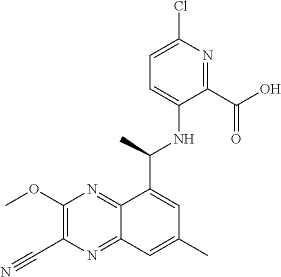 [SMILES: COc1nc2c([C@@H](C)Nc3ccc(Cl)nc3C(=O)O)cc(C)cc2nc1C#N] |
| 839 | (R)-6-chloro-3-((1-(2-cyano-3- hydroxy-7-methylquinoxalin-5- yl)ethyl)amino)picolinic acid | 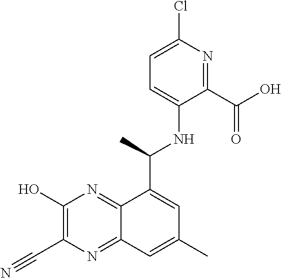 [SMILES: Cc1cc([C@@H](C)Nc2ccc(Cl)nc2C(=O)O)c2nc(O)c(C#N)nc2c1] |
| 840 | (R)-6-chloro-3-((1-(2-cyano-3- (diethylamino)-7-methylquinoxalin-5- yl)ethyl)amino)picolinic acid | 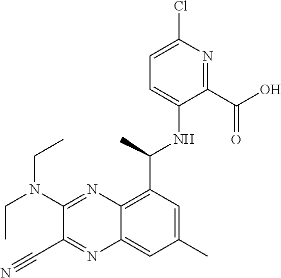 [SMILES: CCN(CC)c1nc2c([C@@H](C)Nc3ccc(Cl)nc3C(=O)O)cc(C)cc2nc1C#N] |
| 841 | 6-chloro-3-(((1R)-1-(2-cyano-7- methyl-3-((1-(pyridin-3- yl)ethyl)amino)quinoxalin-5- yl)ethyl)amino)picolinic acid | 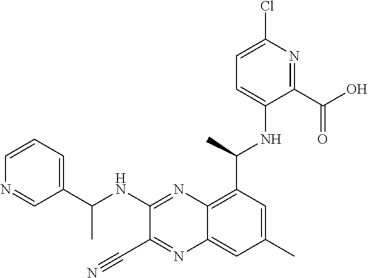 [SMILES: Cc1cc([C@@H](C)Nc2ccc(Cl)nc2C(=O)O)c2nc(NC(C)c3cccnc3)c(C#N)nc2c1] |
| 842 | 6-chloro-3-(((1R)-1-(2-cyano-7- methyl-3-((3,3,3-trifluoro-2- methylpropyl)amino)quinoxalin-5- yl)ethyl)amino)picolinic acid | 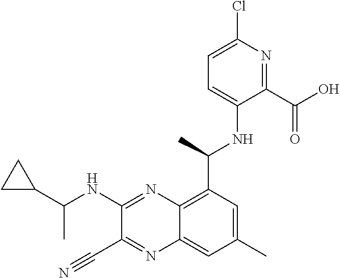 [SMILES: Cc1cc([C@@H](C)Nc2ccc(Cl)nc2C(=O)O)c2nc(NC(C)C3CC3)c(C#N)nc2c1] |
| 843 | 6-chloro-3-(((1R)-1-(2-cyano-3-((1- cyclopropylethyl)amino)-7- methylquinoxalin-5- yl)ethyl)amino)picolinic acid | 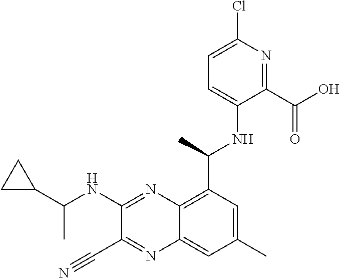 [SMILES: Cc1cc([C@@H](C)Nc2ccc(Cl)nc2C(=O)O)c2nc(NC(C)C3CC3)c(C#N)nc2c1] |
| 844 | (R)-6-chloro-3-((1-(2-cyano-3- (isobutylamino)-7-methylquinoxalin-5- yl)ethyl)amino)picolinic acid | 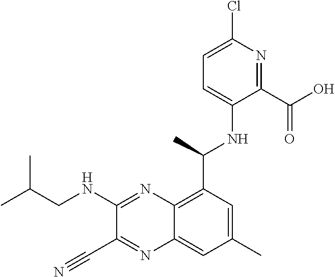 [SMILES: Cc1cc([C@@H](C)Nc2ccc(Cl)nc2C(=O)O)c2nc(NCC(C)C)c(C#N)nc2c1] |
| 845 | 6-chloro-3-(((1R)-1-(2-cyano-3-(3- hydroxy-3-isopropylpyrrolidin-1-yl)-7- methylquinoxalin-5- yl)ethyl)amino)picolinic acid | 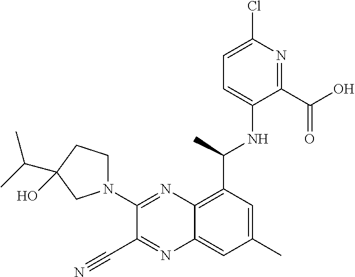 [SMILES: Cc1cc([C@@H](C)Nc2ccc(Cl)nc2C(=O)O)c2nc(N3CCC(O)(C(C)C)C3)c(C#N)nc2c1] |
| 846 | 6-chloro-3-(((1R)-1-(2-cyano-3-(1- isopropyl-7,7-dioxido-7-thia-2- azaspiro[3.5]nonan-2-yl)-7- methylquinoxalin-5- yl)ethyl)amino)picolinic acid | 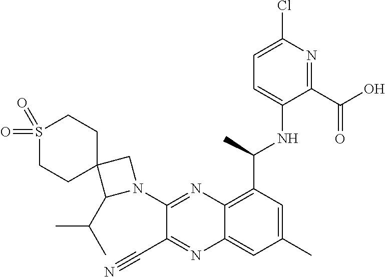 [SMILES: Cc1cc([C@@H](C)Nc2ccc(Cl)nc2C(=O)O)c2nc(N3CC4(CCS(=O)(=O)CC4)C3C(C)C)c(C#N)nc2c1] |

### Examples 847-859

The compounds in Table 4 are prepared essentially according to the procedures set forth in the above schemes and examples.

TABLE 4

| Example | | |
|---|---|---|
| No. | Structure and Name | Spectral Data |
| 847 | 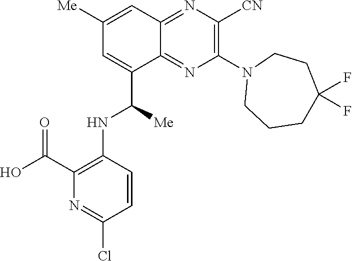 [SMILES: Cc1cc([C@@H](C)Nc2ccc(Cl)nc2C(=O)O)c2nc(N3CCCC(F)(F)CC3)c(C#N)nc2c1] | ¹H NMR (400 MHz, CD₃OD) δ = 7.59 (s, 2H), 7.18 (d, J = 9.2 Hz, 1H), 6.96 (d, J = 9.2 Hz, 1H), 5.52 (q, J = 6.8 Hz, 1H), 4.09- 3.99 (m, 4H), 2.55-2.45 (m, 2H), 2.43 (s, 3H), 2.25-2.10 (m, 4H), 1.70 (d, J = 6.4 Hz, 3H) LCMS [M + 23] +: 523.2. |
| | (R)-6-chloro-3-((1-(2-cyano-3-(4,4- | |
| | difluoroazepan-1-yl)-7- | |
| | methylquinoxalin-5- | |
| | yl)ethyl)amino)picolinic acid | |
| 848 | 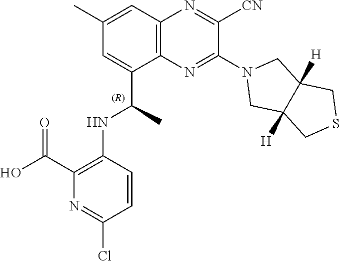 | ¹H NMR (400 MHz, CDCl₃) δ = 8.34 (br d, J = 6.4 Hz, 1H), 7.58 (s, 1H), 7.47 (s, 1H), 7.12 (d, J = 8.8 Hz, 1H), 6.80 (d, J = 8.8 Hz, 1H), 5.44 (quin, J = 6.8 Hz, 1H), 4.30-4.16 (m, 2H), 3.87 (ddd, J = 3.6, 11.6, 12.0 Hz, 2H), 3.31-3.14 (m, 4H), 2.99-2.82 (m, 2H), 2.43 (s, 3H), 1.70 (d, J = 6.8 Hz, 3H). LCMS [M + Na]⁺: 517.1. |
| | 6-chloro-3-(((R)-1-(2-cyano-7-methyl- | |
| | 3-cis-tetrahydro-1h-thieno[3,4- | |
| | c]pyrrol-5(3H)-ylquinoxalin-5- | |
| | yl)ethyl)amino)picolinic acid | |
| 849 | 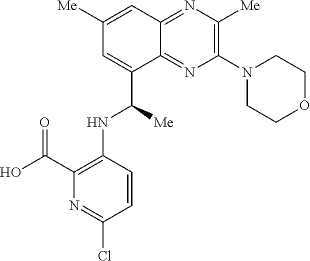 [SMILES: Cc1cc([C@@H](C)Nc2ccc(Cl)nc2C(=O)O)c2nc(N3CCOCC3)c(C)nc2c1] | ¹H NMR (400 MHz, DMSO-d₆) δ = 12.95 (s, 1H), 8.55 (br d, J = 7.2 Hz, 1H), 7.55 (s, 1H), 7.45 (d, J = 1.6 Hz, 1H), 7.31 (d, J = 9.2 Hz, 1H), 7.11 (d, J = 9.2 Hz, 1H), 5.49 (quin, J = 6.8 Hz, 1H), 3.81 (t, J = 4.8 Hz, 4H), 3.44-3.32 (m, 4H), 2.65 (s, 3H), 2.41 (s, 3H), 1.67 (d, J = 6.8 Hz, 3H). LCMS [M + H]⁺: 442.3. |
| | (R)-6-chloro-3-((1-(2,7-dimethyl-3- | |
| | morpholinoquinoxalin-5- | |
| | yl)ethyl)amino)picolinic acid | |
| 850 | 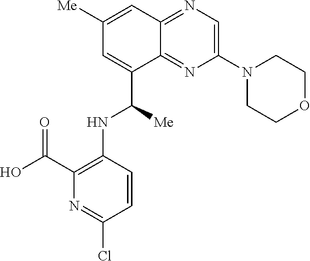 [SMILES: Cc1cc([C@@H](C)Nc2ccc(Cl)nc2C(=O)O)c2nc(N3CCOCC3)cnc2c1] | ¹H NMR (400 MHz, DMSO-d₆) δ = 8.94 (s, 1H), 8.81 (s, 1H), 7.53 (d, J = 0.8 Hz, 1H), 7.41 (d, J = 1.6 Hz, 1H), 7.22 (d, J = 8.8 Hz, 1H), 6.94 (br d, J = 8.8 Hz, 1H), 5.59-5.29 (m, 1H), 3.99-3.56 (m, 8H), 2.37 (s, 3H), 1.61 (d, J = 6.8 Hz, 3H). LCMS [M + 1]⁺: 428.1. |
| | (R)-6-chloro-3-((1-(7-methyl-3- | |
| | morpholinoquinoxalin-5- | |
| | yl)ethyl)amino)picolinic acid | |
| 851 | 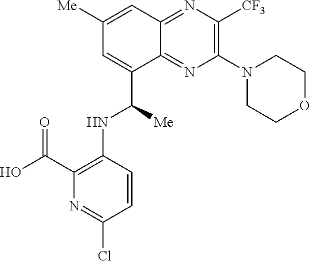 [SMILES: Cc1cc([C@@H](C)Nc2ccc(Cl)nc2C(=O)O)c2nc(N3CCOCC3)c(C(F)(F)F)nc2c1] | ¹H NMR (400 MHz, CDCl₃) δ = 10.71 (s, 1H), 8.37 (br d, J = 6.0 Hz, 1H), 7.76 (s, 1H), 7.55 (d, J = 1.6 Hz, 1H), 7.11 (d, J = 8.8 Hz, 1H), 6.81 (d, J = 8.8 Hz, 1H), 5.57 (quin, J = 6.8 Hz, 1H), 4.03-3.85 (m, 4H), 3.61-3.43 (m, 4H), 2.49 (s, 3H), 1.73 (d, J = 6.8 Hz, 3H). LCMS [M + 1] ⁺= 496.2. |
| | (R)-6-chloro-3-((1-(7-methyl-3- | |
| | morpholino-2- | |
| | (trifluoromethyl)quinoxalin-5- | |
| | yl)ethyl)amino)picolinic acid | |
| 852 | 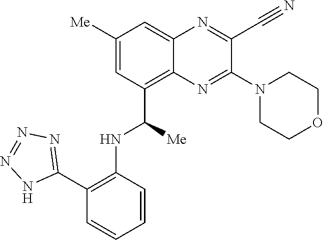 [SMILES: Cc1cc([C@@H](C)Nc2ccccc2-c2nnn[nH]2)c2nc(N3CCOCC3)c(C#N)nc2c1] | ¹H NMR (400 MHz, CD₃OD) δ = 7.77-7.67 (m, 2H), 7.63 (s, 1H), 7.22-7.06 (m, 1H), 6.68 (t, J = 7.2 Hz, 1H), 6.55 (d, J = 8.4 Hz, 1H), 5.66 (q, J = 6.8 Hz, 1H), 4.00-3.88 (m, 4H), 3.79 (dd, J = 4.0, 5.6 Hz, 4H), 2.42 (s, 3H), 1.76 (d, J = 6.8 Hz, 3H). LCMS [M + 1]⁺ = 442.2. |
| | (R)-5-(1-((2-(1H-tetrazol-5- | |
| | yl)phenyl)amino)ethyl)-7-methyl-3- | |
| | morpholinoquinoxaline-2-carbonitrile | |
| 853 | 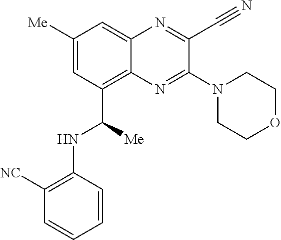 [SMILES: Cc1cc([C@@H](C)Nc2ccccc2C#N)c2nc(N3CCOCC3)c(C#N)nc2c1] | ¹H NMR (400 MHz, CD₃OD) δ = 7.73 (d, J = 1.6 Hz, 1H), 7.64 (s, 1H), 7.37 (dd, J = 1.2, 7.6 Hz, 1H), 7.23 (ddd, J = 1.6, 7.2, 8.8 Hz, 1H), 6.68-6.55 (m, 2H), 5.51 (q, J = 6.8 Hz, 1H), 4.00-3.89 (m, 4H), 3.87-3.70 (m, 4H), 2.48 (s, 3H), 1.72 (d, J = 6.8 Hz, 3H). LCMS [M + 1] ⁺ = 421.2. |
| | (R)-5-(1-((2- | |
| | cyanophenyl)amino)ethyl)-7-methyl- | |
| | 3-morpholinoquinoxaline-2- | |
| | carbonitrile | |
| 854 | 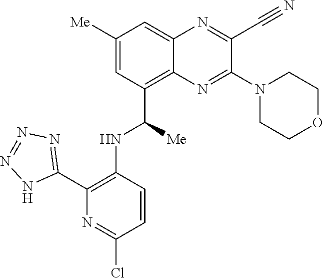 [SMILES: Cc1cc([C@@H](C)Nc2ccc(Cl)nc2-c2nnn[nH]2)c2nc(N3CCOCC3)c(C#N)nc2c1] | ¹H NMR (400 MHz, CD₃OD) δ = 7.70 (d, J = 1.6 Hz, 1H), 7.66 (s, 1H), 7.14 (d, J = 8.8 Hz, 1H), 7.02 (d, J = 8.8 Hz, 1H), 5.62 (q, J = 6.8 Hz, 1H), 4.02-3.87 (m, 4H), 3.84- 3.71 (m, 4H), 2.44 (s, 3H), 1.82 (d, J = 6.8 Hz, 3H). LCMS [M + Na]⁺ = 499.2. |
| | (R)-5-(1-((6-chloro-2-(1H-tetrazol-5- | |
| | yl)pyridin-3-yl)amino)ethyl)-7-methyl- | |
| | 3-morpholinoquinoxaline-2- | |
| | carbonitrile | |
| 855 | 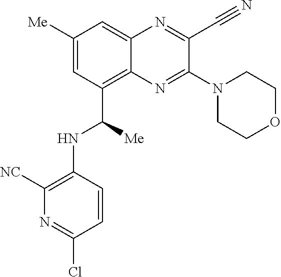 [SMILES: Cc1cc([C@@H](C)Nc2ccc(Cl)nc2C#N)c2nc(N3CCOCC3)c(C#N)nc2c1] | ¹H NMR (400 MHz, CD₃OD) δ = 7.73 (d, J = 1.6 Hz, 1H), 7.66 (d, J = 0.8 Hz, 1H), 7.24 (d, J = 9.2 Hz, 1H), 7.09 (d, J = 9.2 Hz, 1H), 5.53 (q, J = 6.8 Hz, 1H), 3.97-3.88 (m, 4H), 3.84-3.67 (m, 4H), 2.49 (s, 3H), 1.74 (d, J = 6.8 Hz, 3H). LCMS [M + 1]⁺ = 434.2. |
| | (R)-5-(1-((6-chloro-2-cyanopyridin-3- | |
| | yl)amino)ethyl)-7-methyl-3- | |
| | morpholinoquinoxaline-2-carbonitrile | |
| 856 | 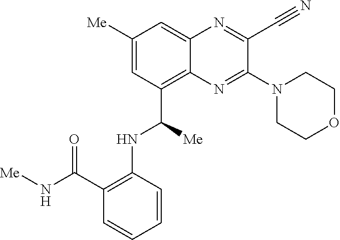 [SMILES: CNC(=O)c1ccccc1N[C@H](C)c1cc(C)cc2nc(C#N)c(N3CCOCC3)nc12] | ¹H NMR (400 MHz, CD₃OD) δ = 7.70 (d, J = 1.6 Hz, 1H), 7.61 (s, 1H), 7.43 (dd, J = 1.2, 7.6 Hz, 1H), 7.07-6.98 (m, 1H), 6.52 (t, J = 7.6 Hz, 1H), 6.37 (d, J = 8.4 Hz, 1H), 5.54 (q, J = 6.4 Hz, 1H), 3.97-3.87 (m, 4H), 3.83-3.74 (m, 4H), 2.91 (s, 3H), 2.44 (s, 3H), 1.64 (d, J = 6.8 Hz, 3H). LCMS [M + 1]⁺ = 431.3. |
| | (R)-2-((1-(2-cyano-7-methyl-3- | |
| | morpholinoquinoxalin-5- | |
| | yl)ethyl)amino)-N-methylbenzamide | |
| 857 | 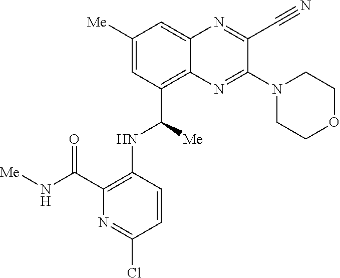 [SMILES: CNC(=O)c1nc(Cl)ccc1N[C@H](C)c1cc(C)cc2nc(C#N)c(N3CCOCC3)nc12] | ¹H NMR (400 MHz, CD₃OD) δ = 8.99 (d, J = 6.8 Hz, 1H), 7.66 (d, J = 1.6 Hz, 1H), 7.63 (dd, J = 0.8, 1.6 Hz, 1H), 7.08 (d, J = 8.8 Hz, 1H), 6.88 (d, J = 8.8 Hz, 1H), 5.56-5.48 (m, 1H), 3.94-3.88 (m, 4H), 3.83-3.72 (m, 4H), 2.92 (s, 3H), 2.45 (s, 3H), 1.69 (d, J = 6.4 Hz, 3H). LCMS [M + 1] ⁺ = 466.2. |
| | (R)-6-chloro-3-((1-(2-cyano-7-methyl- | |
| | 3-morpholinoquinoxalin-5- | |
| | yl)ethyl)amino)-N-methylpicolinamide | |
| 858 | 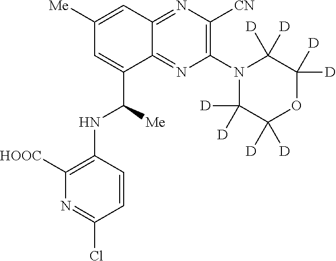 [SMILES: [2H]C1([2H])OC([2H])([2H])C([2H])([2H])N(c2nc3c([C@@H](C)Nc4ccc(Cl)nc4C(=O)O)cc(C)cc3nc2C#N)C1([2H])[2H]] | ¹H NMR (400 MHz, DMSO-d₆) δ = 9.98 (d, J = 7.2 Hz, 1H), 7.65 (s, 1H), 7.59 (s, 1H), 7.04 (d, J = 8.8 Hz, 1H), 6.72 (d, J = 8.8 Hz, 1H), 5.47-5.32 (m, 1H), 2.41 (s, 3H), 1.58 (d, J = 6.8 Hz, 3H). LCMS [M + 1] ⁺ = 461.3. |
| | (R)-6-chloro-3-((1-(2-cyano-7-methyl- | |
| | 3-(morpholino-d8)quinoxalin-5- | |
| | yl)ethyl)amino)picolinic acid | |
| 859 | 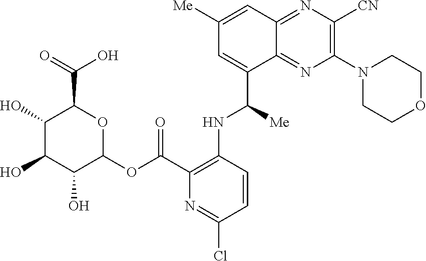 [SMILES: Cc1cc([C@@H](C)Nc2ccc(Cl)nc2C(=O)OC2O[C@H](C(=O)O)[C@@H](O)[C@H](O)[C@H]2O)c2nc(N3CCOCC3)c(C#N)nc2c1] | ¹H NMR (400 MHz, CD₃OD) δ = 7.70 (d, J = 10.3 Hz, 2H), 7.27 (d, J = 9.0 Hz, 1H), 7.11 (d, J = 9.1 Hz, 1H), 5.70 (d, J = 8.0 Hz, 1H), 5.58 (q, J = 6.7 Hz, 1H), 4.94-4.94 (m, 1H), 4.88-4.87 (m, 1H), 3.97-3.75 (m, 9H), 3.70-3.55 (m, 3H), 3.36-3.35 (m, 1H), 2.49 (s, 3H), 1.76 (d, J = 6.8 Hz, 3H). LCMS [M + 1] ⁺ = 629.3. |
| | (2S,3S,4S,5R)-6-((6-chloro-3-(((R)-1- | |
| | (2-cyano-7-methyl-3- | |
| | morpholinoquinoxalin-5- | |
| | yl)ethyl)amino)picolinoyl)oxy)-3,4,5- | |
| | trihydroxytetrahydro-2H-pyran-2- | |
| | carboxylic acid | |

### Example A

This Example illustrates that representative compounds of the invention inhibit the formation of phospho-AKT (pAKT) in a cell.

The ability of a compound of Formula (I) to inhibit the formation of pAKT was measured using alphaLISA Surefire Ultra AKT 1/2/3 (pS473) Assay Kit (#ALSU-PAKT-B50K) was obtained from Perkin Elmer (Waltham, MA).

To prepare assay plates for pAKT alphaLISA assays, cells were trypsinized, resuspended in fresh media, and viable cells were counted utilizing trypan blue exclusion. Prior to seeding, cells were washed with PBS and resuspended in HBSS (Gibco, #14025092). T47D (12,000/w), SKBR3 (12,000/w), or MKN1 (24,000/w) cells were seeded at 12 μl per well in a solid white flat bottom 384 cell culture plate (Perkin Elmer #6007680).

Immediately after seeding, cells were dosed using an Echo Liquid Handler (Beckman Coulter) with compounds at a 10.4 μm starting concentration and serially diluted (1:4) for a total of 10 concentrations. 14 vehicle (DMSO) and 14 positive control (Alpelisib @3.125 μm) wells were included on each assay plate. Cells were incubated with the compounds (solubilized in DMSO) for approximately 24 hours at 37° C. After 24 hours of treatment, cells were lysed with 3 μl of the 5× lysis buffer (provided and incubated at room temperature for 15 min on a microtiter plate shaker). Once cells were sufficiently lysed, 7.5 μl of the acceptor bead mix (made using the manufacturers recommended dilutions) was added to each well and left on the microtiter plate shaker for 1 min prior to incubating plates at room temperature for 1 hour, in the absence of light. After 1 hour of incubation with the acceptor bead mix, 7.5 μl of the donor bead mix (made using the manufacturers recommended dilutions) was added to each well and left on the microtiter plate shaker for 1 min prior to incubating plates at room temperature overnight in the absence of light. Plates were then imaged the following day using a CLARIOstar microplate reader (BMG Labtech, Germany).

Percent of control values were calculated by subtracting the average signal from the positive control (alpelisib) treated wells from all treated wells (including DMSO control wells) and then divided by the average signal from the vehicle DMSO treated control wells. Percent of vehicle control values were plotted as log(inhibitor) vs. response—Variable slope (four parameters) for curve fitting and IC₅₀ values were determined using XLfit.

The results are shown in Tables below. Key: N.D.=not determined

TABLE A
Inhibition of pAKT in T47D
(PI3Kα H1047R mutant)
cells by representative
compounds of Formula (I)

| | T47D (H1047R) |
|---|---|
| Example | pAKT 24 hr |
| No. | (IC50) |
| 1 | 39 |
| 2 | 18.2 |
| 3 | 9.5 |
| 4 | 843.9 |
| 5 | 0.8 |
| 6 | 5 |
| 7 | 0.8 |
| 8 | 7.7 |
| 9 | 7.5 |
| 10 | |
| 11 | |
| 12 | |
| 13 | |
| 14 | |
| 15 | |
| 16 | |
| 17 | |
| 18 | |
| 19 | |
| 20 | |
| 21 | 0.5 |
| 22 | 1.9 |
| 23 | 0.7 |
| 24 | 1.3 |
| 25 | 1.2 |
| 26 | 1.8 |
| 27 | 1142 |
| 28 | 48.3 |
| 29 | 117.4 |
| 30 | 4.7 |
| 31 | 561 |
| 32 | 10.2 |
| 33 | 4.3 |
| 34 | 6.2 |
| 35 | 3.1 |
| 36 | 93.7 |
| 37 | 94.6 |
| 38 | |
| 39 | |
| 40 | |
| 41 | |
| 42 | |
| 43 | |
| 43B | 16.8 |
| 43A | 4.8 |
| 44 | 2 |
| 45 | 6.8 |
| 46A | |
| 46B | |
| 48 | |
| 49 | |
| 50 | |
| 51 | |
| 52 | |
| 53 | |
| 54 | |
| 55 | 21.6 |
| 56 | 4.7 |
| 57 | 14 |
| 58 | 1.8 |
| 59 | |
| 60 | |
| 61 | 38.6 |
| 62 | 7.9 |
| 63 | 16.5 |
| 64 | 1.3 |
| 65 | |
| 66 | |
| 67 | 72 |
| 68 | 24.4 |
| 69 | 2.1 |
| 70 | 2.6 |
| 71 | |
| 72A | |
| 72B | |
| 73 | |
| 74 | |
| 75A | 52 |
| 75B | 39 |
| 76 | |
| 77 | |
| 78A | |
| 78B | |
| 79A | |
| 79B | |
| 80 | |
| 81 | |
| 82 | 4.5 |
| 83 | |
| 84 | |
| 85 | 2 |
| 86 | |
| 87 | |
| 88 | |
| 89 | |
| 90 | |
| 91 | |
| 92A | |
| 92 | |
| 93 | |
| 94 | |
| 94A | |
| 94B | |
| 95A | |
| 95B | |
| 96A | |
| 96B | |
| 97A | |
| 97B | |
| 98A | |
| 98B | |
| 99A | |
| 99B | |
| 100 | |
| 101 | |
| 102 | |
| 103 | |
| 104 | |
| 105 | |
| 106 | |
| 107 | |
| 108 | |
| 109A | |
| 109B | |
| 110A | |
| 110B | |
| 111A | |
| 111B | |
| 112A | |
| 112B | |
| 113 | 10420 |
| 114 | 11 |
| 115 | 1 |
| 116 | 15 |
| 117 | 66 |
| 118 | |
| 119 | |
| 120 | 2.4 |
| 121 | |
| 122 | 1.3 |
| 123 | 66.8 |
| 124 | 1.9 |
| 125 | 2.7 |
| 126 | 0.8 |
| 127 | 8.4 |
| 128 | 1.4 |
| 129 | 18.8 |
| 130 | |
| 131 | 2.2 |
| 132 | 0.9 |
| 133 | 0.8 |
| 134 | 10.9 |
| 135 | 19.2 |
| 136 | 0.7 |
| 137 | 1.3 |
| 138 | 0.8 |
| 139 | 2.1 |
| 140 | |
| 141 | |
| 142 | |
| 143 | |
| 144 | |
| 145 | 1.4 |
| 146 | |
| 147 | |
| 148 | |
| 149 | |
| 150 | |
| 151 | |
| 152 | |
| 153 | |
| 154 | |
| 155 | |
| 156 | |
| 157 | |
| 158 | 3.6 |
| 159 | 2.6 |
| 160 | 4.6 |
| 161 | 4.6 |
| 162 | 1.6 |
| 163 | 6.8 |
| 164 | 2.1 |
| 165 | 5.6 |
| 166 | 1.2 |
| 167 | 4.6 |
| 168 | |
| 169 | |
| 170 | |
| 171 | |
| 172 | |
| 173 | 0.3 |
| 174 | 4.4 |
| 175 | |
| 176 | 1.7 |
| 177 | 0.5 |
| 178 | 0.5 |
| 179 | 3.9 |
| 180 | 1.6 |
| 181 | 1.7 |
| 182A | |
| 182B | |
| 183 | |
| 184 | |
| 185A | |
| 185B | |
| 186 | |
| 187 | |
| 188 | |
| 189A | |
| 189B | |
| 190A | 4.4 |
| 190B | 17.3 |
| 191A | |
| 191B | |
| 192A | |
| 192B | |
| 193 | |
| 194A | |
| 194B | |
| 195A | 0.47 |
| 195B | 0.37 |
| 196A | 1.2 |
| 196B | 2.2 |
| 197 | 1.8 |
| 198 | |
| 199 | |
| 200 | 1.2 |
| 201 | 0.27 |
| 202 | 1.4 |
| 203 | 25.1 |
| 204 | 58.1 |
| 205 | |
| 206 | |
| 207 | |
| 208 | |
| 209 | |
| 210A | 0.69 |
| 210B | 0.8 |
| 211A | 5.7 |
| 211B | 2.6 |
| 212A | |
| 212B | |
| 213 | 0.91 |
| 214 | |
| 215 | 1.6 |
| 216 | 0.45 |
| 217 | 0.69 |
| 218 | |
| 219 | |
| 220 | |
| 221 | 0.88 |
| 222 | 4.2 |
| 223 | 20 |
| 224 | |
| 225 | |
| 226 | |
| 227 | |
| 228 | 33.2 |
| 229A | 0.55 |
| 229B | 1.2 |
| 230 | |
| 231 | 1.3 |
| 232 | |
| 233 | 0.17 |
| 234A | 2.8 |
| 234B | 3.6 |
| 235A | 2 |
| 235B | 1.8 |
| 236A | 0.89 |
| 236B | 3.6 |
| 237A | 4.3 |
| 237B | 6.1 |
| 238A | 10.4 |
| 238B | 3.5 |
| 239A | 1 |
| 239B | 0.4 |
| 240 | 1.6 |
| 241A | |
| 241B | |
| 242 | 0.28 |
| 243A | 0.34 |
| 243B | 0.52 |
| 244A | 1.2 |
| 244B | 1.3 |
| 245 | 1.1 |
| 246A | 0.69 |
| 246B | 1.8 |
| 246C | 0.97 |
| 246D | 6.8 |
| 247A | |
| 247B | |
| 248A | |
| 248B | |
| 249 | 1.7 |
| 250 | 1.2 |
| 251 | 1.1 |
| 252 | 2.7 |
| 253A | 5.6 |
| 253B | 5.8 |
| 254A | |
| 254B | |
| 255 | 3.7 |
| 256 | 0.11 |
| 257A | 4.8 |
| 257B | 6.7 |
| 258 | 2.99 |
| 259 | 1.5 |
| 260 | 1.9 |
| 261A | 3.2 |
| 261B | 2.6 |
| 261C | 2.1 |
| 261D | 1.2 |
| 847 | 1.8 |
| 848 | 1.7 |
| 852 | |
| 853 | |
| 854 | |
| 855 | |
| 856 | |
| 857 | |

TABLE B
Inhibition of pAKT in
SKBR3 (PI3Kα wild type)
cells by representative
compounds of Formula (I)

| Example | SKBR3 (WT) |
|---|---|
| No. | pAKT IC₅₀ (nM) |
| 1 | >10000 |

### Example B

This Example illustrates that representative compounds of the invention decrease the viability of cells.

The ability of a compound of Formula (I) to decrease the viability of cells was measured using CellTiter-Glo 2.0 (CTG) Luminescent Cell Viability Assay (#G9241) obtained from Promega (Madison, WI).

To prepare assay plates for viability assays, cells were trypsinized, resuspended in fresh media, and viable cells were counted utilizing trypan blue exclusion. T47D, SKBR3, or MKN1 cells were seeded at 1000 cells in 30 μl per well in a solid white flat bottom 384 cell culture plate (Perkin Elmer #6007680) and incubated at 37° C. overnight.

Assay day 1, cells were dosed using an Echo Liquid Handler (Beckman Coulter) with compounds at a 10 μm starting concentration and serially diluted (1:4) for a total of 10 concentrations. Cells were incubated for approximately 72 hours with the compounds (solubilized in DMSO) at 37° C. After 72 hours of treatment, cell plates were equilibrated to room temperature before adding 15 μl of CTG to each well, plates were then covered in aluminum foil to protect from light, incubated at room temperature for 30 minutes on a microtiter plate shaker, and luminescence readings were collected using a CLARIOstar microplate reader (BMG Labtech, Germany). Percent of vehicle control values were plotted as log(inhibitor) vs. response—Variable slope (four parameters) for curve fitting and IC₅₀ values were determined using XLfit.

TABLE C
Viability in T47D (PI3Kα
H1047R mutant) cells by
representative compounds
of Formula (I)

| | T47D |
|---|---|
| | (H1047R mutant) |
| Example | Viability IC₅₀ |
| No. | (nM) |
| 1 | 456.4 |
| 2 | 631.4 |
| 3 | 170.4 |
| 4 | 9328 |
| 5 | 37.3 |
| 6 | 294.2 |
| 7 | 59.8 |
| 8 | 34.1 |
| 9 | 65.1 |
| 10 | 71.6 |
| 11 | 64 |
| 12 | 159.9 |
| 13 | |
| 14 | |
| 15 | 119.5 |
| 16 | |
| 17 | |
| 18 | |
| 19 | |
| 20 | |
| 21 | 32.1 |
| 22 | 79.3 |
| 23 | 61.9 |
| 24 | 44.2 |
| 25 | 175 |
| 26 | 51.5 |
| 27 | 10000 |
| 28 | 2142 |
| 29 | 3477 |
| 30 | 183 |
| 31 | 10000 |
| 32 | 264.7 |
| 33 | 91.4 |
| 34 | 48.2 |
| 35 | 166.2 |
| 36 | 1170 |
| 37 | 588 |
| 38 | |
| 39 | 2681 |
| 40 | |
| 41 | |
| 42 | |
| 43 | |
| 43B | 271.1 |
| 43A | 99 |
| 44 | 156.6 |
| 45 | 115.4 |
| 46A | |
| 46B | |
| 48 | 95.3 |
| 49 | |
| 50 | |
| 51 | |
| 52 | |
| 53 | |
| 54 | |
| 55 | 422 |
| 56 | 128.3 |
| 57 | 164.6 |
| 58 | 24 |
| 59 | 140.4 |
| 60 | 21.1 |
| 61 | 512.6 |
| 62 | 219.7 |
| 63 | 254.4 |
| 64 | 44 |
| 65 | 455.7 |
| 66 | 78 |
| 67 | 686.6 |
| 68 | 391.4 |
| 69 | 50.2 |
| 70 | 31 |
| 71 | 276 |
| 72A | 297.7 |
| 72B | 96.3 |
| 73 | |
| 74 | 95 |
| 75A | 701 |
| 75B | 720 |
| 76 | 324 |
| 77 | 77 |
| 78A | 125.5 |
| 78B | 84.4 |
| 79A | 251.8 |
| 79B | 176.5 |
| 80 | |
| 81 | |
| 82 | 121 |
| 83 | 60.7 |
| 84 | 343 |
| 85 | |
| 86 | 270 |
| 87 | 88.7 |
| 88 | 365 |
| 89 | 63.2 |
| 90 | |
| 91 | |
| 92A | |
| 92 | |
| 93 | |
| 94 | |
| 94A | |
| 94B | |
| 95A | |
| 95B | |
| 96A | |
| 96B | |
| 97A | |
| 97B | |
| 98A | |
| 98B | |
| 99A | |
| 99B | |
| 100 | |
| 101 | |
| 102 | |
| 103 | |
| 104 | |
| 105 | |
| 106 | 98.1 |
| 107 | 215.6 |
| 108 | |
| 109A | |
| 109B | |
| 110A | 297.7 |
| 110B | 96.3 |
| 111A | |
| 111B | |
| 112A | |
| 112B | |
| 113 | 1169 |
| 114 | 189.6 |
| 115 | 31.3 |
| 116 | 201.5 |
| 117 | 497.8 |
| 118 | 382.6 |
| 119 | 59.9 |
| 120 | 56.4 |
| 121 | 651 |
| 122 | 33.8 |
| 123 | 273 |
| 124 | 126.2 |
| 125 | 163 |
| 126 | 24.8 |
| 127 | 296.3 |
| 128 | 120.6 |
| 129 | 330.4 |
| 130 | 65.6 |
| 131 | |
| 132 | |
| 133 | |
| 134 | |
| 135 | |
| 136 | |
| 137 | |
| 138 | |
| 139 | |
| 140 | |
| 141 | |
| 142 | |
| 143 | |
| 144 | |
| 145 | 90 |
| 146 | |
| 147 | |
| 148 | |
| 149 | |
| 150 | |
| 151 | |
| 152 | |
| 153 | |
| 154 | |
| 155 | |
| 156 | |
| 157 | |
| 158 | 88.1 |
| 159 | 70.3 |
| 160 | 85.8 |
| 161 | 47.6 |
| 162 | 35 |
| 163 | 190.9 |
| 164 | 55 |
| 165 | 97 |
| 166 | 38.6 |
| 167 | 150 |
| 168 | 45.8 |
| 169 | 108.5 |
| 170 | 93.3 |
| 171 | 177.5 |
| 172 | 169.8 |
| 173 | 26.8 |
| 174 | 36.6 |
| 175 | 344.1 |
| 176 | 34.5 |
| 177 | 19.4 |
| 178 | 24.4 |
| 179 | 77.3 |
| 180 | 57.8 |
| 181 | 44 |
| 182A | |
| 182B | |
| 183 | |
| 184 | |
| 185A | |
| 185B | |
| 186 | |
| 187 | |
| 188 | |
| 189A | |
| 189B | |
| 190A | 271 |
| 190B | 764 |
| 191A | |
| 191B | |
| 192A | |
| 192B | |
| 193 | |
| 194A | |
| 194B | |
| 195A | 84.1 |
| 195B | 100.1 |
| 196A | 62.1 |
| 196B | 61.4 |
| 197 | 105.3 |
| 198 | |
| 199 | |
| 200 | 17.2 |
| 201 | 10.5 |
| 202 | 76.4 |
| 203 | 559 |
| 204 | 393 |
| 205 | |
| 206 | |
| 207 | |
| 208 | |
| 209 | |
| 210A | 135.1 |
| 210B | 120.8 |
| 211A | 78 |
| 211B | 2.7 |
| 212A | |
| 212B | |
| 213 | 61 |
| 214 | |
| 215 | 28 |
| 216 | 48 |
| 217 | 64 |
| 218 | |
| 219 | |
| 220 | |
| 221 | 147 |
| 222 | 225 |
| 223 | 1026 |
| 224 | |
| 225 | |
| 226 | |
| 227 | |
| 228 | 368.2 |
| 229A | 16.6 |
| 229B | 18.6 |
| 230 | |
| 231 | 25.8 |
| 232 | |
| 233 | 84.4 |
| 234A | 73 |
| 234B | 85 |
| 235A | 183 |
| 235B | 262 |
| 236A | 52.8 |
| 236B | 217 |
| 237A | 137 |
| 237B | 152 |
| 238A | 216.4 |
| 238B | 116.7 |
| 239A | 39 |
| 239B | 22 |
| 240 | 16 |
| 241A | |
| 241B | |
| 242 | 35 |
| 243A | 62 |
| 243B | 50 |
| 244A | 57 |
| 244B | 89 |
| 245 | 63 |
| 246A | 35 |
| 246B | 61 |
| 246C | 46 |
| 246D | 158 |
| 247A | |
| 247B | |
| 248A | |
| 248B | |
| 249 | 16 |
| 250 | 37 |
| 251 | 11 |
| 252 | 119 |
| 253A | 197 |
| 253B | 113 |
| 254A | |
| 254B | |
| 255 | 170 |
| 256 | 310 |
| 257A | 139 |
| 257B | 189 |
| 258 | 86 |
| 259 | 75 |
| 260 | 73.8 |
| 261A | 79 |
| 261B | 98 |
| 261C | 78 |
| 261D | 76 |
| 847 | 59 |
| 848 | 90 |
| 852 | 1435 |
| 853 | 4337 |
| 854 | 52 |
| 855 | 1078 |
| 856 | 3445 |
| 857 | 722 |

TABLE D
Viability in SKBR3
(PI3Kα wild type)
cells by representative
compounds of Formula (I)

| | SKBR3 (WT) |
|---|---|
| Example | Viability IC₅₀ |
| No. | (nM) |
| 1 | 10000 |
| 2 | 10000 |
| 3 | 10000 |
| 4 | 10000 |
| 5 | 3967 |
| 6 | 10000 |
| 7 | 10000 |
| 8 | 3617 |
| 9 | 3405 |
| 10 | 10000 |
| 11 | 10000 |
| 12 | 10000 |
| 13 | |
| 14 | |
| 15 | 10000 |
| 16 | |
| 17 | |
| 18 | |
| 19 | |
| 20 | |
| 21 | 10000 |
| 22 | 5320 |
| 23 | 10000 |
| 24 | 10000 |
| 25 | 10000 |
| 26 | 9802 |
| 27 | 10000 |
| 28 | 10000 |
| 29 | 10000 |
| 30 | 10000 |
| 31 | 10000 |
| 32 | 10000 |
| 33 | 7433 |
| 34 | 10000 |
| 35 | 8287 |
| 36 | 10000 |
| 37 | 10000 |
| 38 | |
| 39 | 6231 |
| 40 | |
| 41 | |
| 42 | |
| 43 | |
| 43B | 4032 |
| 43A | 10000 |
| 44 | 10000 |
| 45 | 10000 |
| 46A | |
| 46B | |
| 48 | 10000 |
| 49 | |
| 50 | |
| 51 | |
| 52 | |
| 53 | |
| 54 | |
| 55 | 10000 |
| 56 | 10000 |
| 57 | 10000 |
| 58 | 3502 |
| 59 | 10000 |
| 60 | 10000 |
| 61 | 10000 |
| 62 | 7084 |
| 63 | 9806 |
| 64 | 10000 |
| 65 | 10000 |
| 66 | 7678 |
| 67 | 2774 |
| 68 | 10000 |
| 69 | 9650 |
| 70 | 9650 |
| 71 | 10000 |
| 72A | 10000 |
| 72B | 10000 |
| 73 | |
| 74 | 10000 |
| 75A | 10000 |
| 75B | 10000 |
| 76 | 10000 |
| 77 | 8611 |
| 78A | 7061 |
| 78B | 10000 |
| 79A | 10000 |
| 79B | 10000 |
| 80 | |
| 81 | |
| 82 | 5028 |
| 83 | 10000 |
| 84 | 10000 |
| 85 | |
| 86 | 10000 |
| 87 | 9253 |
| 88 | 10000 |
| 89 | 10000 |
| 90 | |
| 91 | |
| 92A | |
| 92 | |
| 93 | |
| 94 | |
| 94A | |
| 94B | |
| 95A | |
| 95B | |
| 96A | |
| 96B | |
| 97A | |
| 97B | |
| 98A | |
| 98B | |
| 99A | |
| 99B | |
| 100 | |
| 101 | |
| 102 | |
| 103 | |
| 104 | |
| 105 | |
| 106 | 10000 |
| 107 | 10000 |
| 108 | |
| 109A | |
| 109B | |
| 110A | 10000 |
| 110B | 10000 |
| 111A | |
| 111B | |
| 112A | |
| 112B | |
| 113 | |
| 114 | |
| 115 | |
| 116 | |
| 117 | |
| 118 | |
| 119 | |
| 120 | |
| 121 | |
| 122 | |
| 123 | |
| 124 | |
| 125 | |
| 126 | |
| 127 | |
| 128 | |
| 129 | |
| 130 | |
| 131 | |
| 132 | |
| 133 | |
| 134 | |
| 135 | |
| 136 | |
| 137 | |
| 138 | |
| 139 | |
| 140 | |
| 141 | |
| 142 | |
| 143 | |
| 144 | |
| 145 | 9955 |
| 146 | |
| 147 | |
| 148 | |
| 149 | |
| 150 | |
| 151 | |
| 152 | |
| 153 | |
| 154 | |
| 155 | |
| 156 | |
| 157 | |
| 158 | |
| 159 | |
| 160 | |
| 161 | |
| 162 | |
| 163 | |
| 164 | |
| 165 | |
| 166 | |
| 167 | |
| 168 | |
| 169 | |
| 170 | |
| 171 | |
| 172 | |
| 173 | |
| 174 | |
| 175 | |
| 176 | |
| 177 | |
| 178 | |
| 179 | |
| 180 | |
| 181 | |
| 182A | |
| 182B | |
| 183 | |
| 184 | |
| 185A | |
| 185B | |
| 186 | |
| 187 | |
| 188 | |
| 189A | |
| 189B | |
| 190A | 10000 |
| 190B | 10000 |
| 191A | |
| 191B | |
| 192A | |
| 192B | |
| 193 | |
| 194A | |
| 194B | |
| 195A | 10000 |
| 195B | 10000 |
| 196A | 10000 |
| 196B | 10000 |
| 197 | 10000 |
| 198 | |
| 199 | |
| 200 | 5468 |
| 201 | 7809 |
| 202 | 9925 |
| 203 | 2225 |
| 204 | 1853 |
| 205 | |
| 206 | |
| 207 | |
| 208 | |
| 209 | |
| 210A | 10000 |
| 210B | 10000 |
| 211A | 6902 |
| 211B | 6805 |
| 212A | |
| 212B | |
| 213 | 9552 |
| 214 | |
| 215 | 10000 |
| 216 | 10000 |
| 217 | 10000 |
| 218 | |
| 219 | |
| 220 | |
| 221 | 10000 |
| 222 | 10000 |
| 223 | 10000 |
| 224 | |
| 225 | |
| 226 | |
| 227 | |
| 228 | 10000 |
| 229A | 3080 |
| 229B | 4923 |
| 230 | |
| 231 | 10000 |
| 232 | |
| 233 | 10000 |
| 234A | 10000 |
| 234B | 10000 |
| 235A | 10000 |
| 235B | 10000 |
| 236A | 10000 |
| 236B | 10000 |
| 237A | 8170 |
| 237B | 3799 |
| 238A | 10000 |
| 238B | 10000 |
| 239A | 10000 |
| 239B | 7256 |
| 240 | 7986 |
| 241A | |
| 241B | |
| 242 | 8259 |
| 243A | 2861 |
| 243B | 10000 |
| 244A | 10000 |
| 244B | 10000 |
| 245 | 5512 |
| 246A | 10000 |
| 246B | 4916 |
| 246C | 3399 |
| 246D | 6216 |
| 247A | |
| 247B | |
| 248A | |
| 248B | |
| 249 | 6652 |
| 250 | 4888 |
| 251 | 10000 |
| 252 | 5265 |
| 253A | 6848 |
| 253B | 5139 |
| 254A | |
| 254B | |
| 255 | 10000 |
| 256 | 10000 |
| 257A | 3738 |
| 257B | 10000 |
| 258 | 8705 |
| 259 | 10000 |
| 260 | |
| 261A | 10000 |
| 261B | 10000 |
| 261C | 10000 |
| 261D | 10000 |
| 847 | 4937 |
| 848 | 7679 |
| 852 | 10000 |
| 853 | 10000 |
| 854 | 4380 |
| 855 | 6895 |
| 856 | 3564 |
| 857 | 2014 |

It is understood that the examples and embodiments described herein are for illustrative purposes only and that various modifications or changes in light thereof will be suggested to persons skilled in the art and are to be incorporated within the spirit and purview of this application and scope of the appended claims. All publications, patents, and patent applications cited herein are hereby incorporated herein by reference for all purposes.

1. A compound of formula (I): 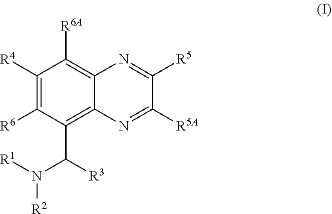
or a pharmaceutically acceptable salt or deuterated form thereof, wherein:
R¹ is H, C₁-C₃ alkyl, or C₃-C₆ cycloalkyl;
R² is phenyl or a 5-6 membered heteroaryl group, wherein each phenyl and heteroaryl is optionally substituted with 1-5 R¹²;
each R¹² is independently C₁-C₄ alkyl, —OR^{A}, —C(O)OR^{A}, (C₁-C₃ alkyl)-OR^{A}, —C(O)N(R^{A})₂, cyano, or halogen;
each R^{A} is independently H, C₁-C₆ alkyl, C₃-C₆ cycloalkyl or 2-carboxy-3,4,5-trihydroxytetrahydro-2H-pyran-6-yl;
R³ is C₁-C₃ alkyl optionally substituted, polysubstituted or persubstituted with fluoro, C₃-C₆ cycloalkyl, C₁-C₃ alkenyl, C₂-C₃ alkynyl, or C₅-C₆ cycloalkenyl;
R⁴ is H, halogen, C₁-C₃ alkyl, C₁-C₆ alkoxy, C₃-C₆ cycloalkyl, C₁-C₃ alkenyl, C₂-C₃ alkynyl, or C₅-C₆ cycloalkenyl or cyano, wherein each C₁-C₃ alkyl and C₃-C₆ cycloalkyl is optionally substituted with 1-5 halo groups;
R⁶ and R^{6A} independently represent H or C₁-C₆ alkyl;
R⁵ is hydrogen, —OR²⁰, —SR²⁰, C₁-C₆ alkyl, halogen, hydroxy, cyano, haloC₁-C₃ alkyl, hydroxy C₁-C₆ alkyl, C₁-C₆ alkoxy, C₁-C₆ alkoxy(C₁-C₆)alkyl, cyano, amino, mono- or di(C₁-C₃) alkyl amino, C₃-C₆ cycloalkyl, C₃-C₆ cycloalkyl(C₁-C₃ alkyl), 3-8 membered heterocyclyl, phenyl or 5-6 membered heteroaryl, wherein each alkyl, alkenyl, cycloalkyl, phenyl, 3-8 membered heterocyclyl and heteroaryl is optionally independently substituted with 1-3 of halogen, hydroxy, C₁-C₃ alkyl, C₁-C₃ alkoxy, haloC₁-C₃ alkyl, hydroxy C₁-C₆ alkyl, oxo, cyano, amino, mono-ordi(C₁-C₃) alkyl amino,
wherein R²⁰ is aryl, aryl(C₁-C₆)alkyl, 5-7 membered heteroaryl or 5-7 membered heteroaryl(C₁-C₆)alkyl, wherein each aryl and heteroaryl is optionally independently substituted with one to four of C₁-C₆ alkyl, halogen, hydroxy, cyano, haloC₁-C₃ alkyl, hydroxy C₁-C₆ alkyl, C₁-C₆ alkoxy, C₁-C₆ alkoxy(C₁-C₆)alkyl, cyano, amino, or mono- or di(C₁-C₃) alkyl amino;
R^{5A} represents NR⁷R⁸, C₁-C₆ alkyl, C₃-C₆ cycloalkyl, C₃-C₆ cycloalkyl(C₁-C₃ alkyl), phenyl, or 5-6 membered heteroaryl, wherein each C₁-C₆ alkyl, C₃-C₆ cycloalkyl, C₃-C₆ cycloalkyl(C₁-C₃ alkyl), phenyl and heteroaryl is optionally independently substituted with 1-3 of halogen, halo(C₁-C₃ alkyl), hydroxy, oxo, C₁-C₃ alkyl, C₁-C₃ alkoxy, C₁-C₆ alkanoyl, cyano, amino, or mono- or di(C₁-C₃) alkyl amino;
R⁷ represents H or C₁-C₃ alkyl;
R⁸ represents H, C₁-C₆ alkyl, C₂-C₆ alkenyl, C₃-C₆ cycloalkyl, C₃-C₆ cycloalkyl(C₁-C₃ alkyl), 3-8 membered heterocyclyl, 3-8 membered heterocyclyl(C₁-C₆) alkyl, phenyl(C₁-C₆)alkyl or 5-6 membered heteroaryl(C₁-C₆)alkyl, wherein each C₁-C₆ alkyl, C₃-C₆ cycloalkyl, C₃-C₆ cycloalkyl(C₁-C₃ alkyl), phenyl, heteroaryl and 3-8 membered heterocyclyl is optionally independently substituted with 1-3 of halogen, halo(C₁-C₃ alkyl), hydroxy, oxo, C₁-C₃ alkyl, C₁-C₃ alkoxy, cyano, amino, mono- or di(C₁-C₃) alkyl amino, amino- or mono- or di(C₁-C₃) alkylaminocarbonyl, —SO₂R¹⁶, —SO₂(NR¹⁶)₂, or —NR¹⁷SO₂R¹⁶ wherein each R¹⁶ is independently C₁-C₆ alkyl and R¹⁷ is C₁-C₆ alkyl; or
R⁷ and R⁸ together with the nitrogen to which they are attached form ring Q, wherein ring Q is
a 3-8 membered monocyclic heterocyclyl group, wherein the heterocyclyl group
is optionally fused to an aromatic or non-aromatic ring containing 3-6 ring members of which 1 or 2 are optionally nitrogen, oxygen or sulfur atoms and the remainder carbons, or
with a carbon atom from the monocyclic heterocyclyl group or fused ring system and additional carbon or hetero atoms, forms a spirocyclic ring system having up to 10 total ring members, wherein the ring members are carbons or 1 or 2 hetero atoms selected from nitrogen, sulfur and oxygen atoms, and
wherein each ring Q is optionally substituted, on an aromatic or non-aromatic portion, with 1-4 R⁹ groups, wherein each R⁹ is independently (i) C₁-C₆ alkyl optionally substituted with C₁-C₃ alkoxy, (ii) C₁-C₆ alkoxy, (iii) C₃-C₆ cycloalkyl, (iv) cyano, (ii) hydroxy, (v) oxo, (vi) halogen, (vii) halo C₁-C₃ alkyl, (viii) hydroxy C₁-C₆ alkyl, (ix) amino, (x) mono- or di-(C₁-C₆ alkyl) amino where one alkyl is optionally substituted with oxo, (xi) C₁-C₆ alkanoyl optionally substituted with 1 or 2 of C₁-C₃ alkyl, C₁-C₃ alkoxy, C₃-C₆ cycloalkyl, cyano, hydroxy, oxo, halogen, halo C₁-C₃ alkyl, hydroxy C₁-C₃ alkyl, amino, mono- or di-(C₁-C₆ alkyl) amino, C₃-C₆ cycloalkyl, phenyl, 5-6 membered heteroaryl, wherein each C₁-C₃ alkyl and C₃-C₆ cycloalkyl on the alkanoyl is optionally substituted with halogen, hydroxy or amino, and wherein each cycloalkyl, phenyl and heteroaryl on the alkanoyl is optionally substituted with C₁-C₆ alkyl, C₁-C₆ alkoxy, halogen, amino, hydroxy, or cyano, (xii) C₃-C₆ cycloalkyloxy, (xiii) phenyl, (xiv) phenyl(C₁-C₃ alkanoyl), (xv) 5-7 membered heterocyclyl, or (xvi) 5-6 membered heteroaryl, wherein each phenyl, 5-7 membered heterocyclyl and 5-6 membered heteroaryl is optionally substituted with one to four C₁-C₃ alkyl, C₁-C₃ alkoxy, halogen, hydroxy, cyano, oxo, amino, mono- or di(C₁-C₃ alkyl)amino, amino(C₁-C₃) alkyl, mono- or di(C₁-C₃ alkyl)amino(C₁-C₃) alkyl, or amino- or mono- or di(C₁-C₃) alkylaminocarbonyl.

2. A compound according to claim 1, wherein R⁶ and R^{6A} represent hydrogen, one of R⁶ and R^{6A} represents hydrogen and the other represents C₁-C₆ alkyl, or R⁶ and R^{6A} independently represent C₁-C₆ alkyl.

3. (canceled)

4. (canceled)

5. (canceled)

6. (canceled)

7. (canceled)

8. A compound according to claim 1, wherein R¹ is H or CH₃.

9. (canceled)

10. A compound according to claim 1, wherein R² is phenyl or a 5-7 membered heteroaryl, each of which is substituted with 1, 2 or 3 R¹² groups, and at least one R¹² group is —C(O)OR^{A}.

11. (canceled)

12. (canceled)

13. A compound according to claim 1, wherein R² is phenyl or pyridinyl, each of which is optionally substituted with 1-5 R¹², and at least one R¹² group is —C(O)OR^{A}.

14. A compound according to claim 1, wherein R³ is C₁-C₃ alkyl, wherein the alkyl group is unsubstituted, substituted with 1-5 halo groups, or perfluorinated.

15. (canceled)

16. (canceled)

17. A compound according to claim 1, wherein R³ is ethyl or R³ is methyl.

18. A compound according to claim 1, wherein R⁵ is hydrogen, C₁-C₃ alkyl, haloC₁-C₃ alkyl, halogen, cyano, C₃-C₆ cycloalkyl, or C₃-C₆ cycloalkyl(C₁-C₃ alkyl).

19. (canceled)

20. (canceled)

21. A compound according to claim 1, wherein R⁵ is methyl, ethyl, cyclopropyl or cyano.

22. (canceled)

23. (canceled)

24. (canceled)

25. (canceled)

26. (canceled)

27. (canceled)

28. (canceled)

29. A compound according to claim 1, wherein R⁴ is hydrogen, fluoro, chloro, methyl, ethyl, trifluoromethyl or cyano.

30. (canceled)

31. (canceled)

32. (canceled)

33. A compound according to claim 1, wherein R^{5A} represents C₁-C₆ alkyl, C₃-C₆ cycloalkyl or C₃-C₆ cycloalkyl(C₁-C₃ alkyl);

34. A compound according to claim 1, wherein R^{5A} represents NR⁷R⁸.

35. (canceled)

36. A compound according to claim 34,
wherein R⁷ and R⁸ together with the nitrogen to which they are attached form ring Q, wherein ring Q is
a 3-7 membered monocyclic heterocyclyl group, and
the heterocyclyl group is optionally substituted with 1-4 R⁹ groups, wherein each R⁹ is independently C₁-C₆ alkyl, C₁-C₆ alkoxy, C₃-C₆ cycloalkyl, cyano, hydroxy, oxo, halogen, halo C₁-C₃ alkyl, hydroxy C₁-C₆ alkyl, amino, mono- or di-(C₁-C₆ alkyl) amino, or phenyl or phenyl(C₁-C₃ alkanoyl) where each phenyl is optionally substituted with C₁-C₃ alkyl, C₁-C₃ alkoxy, halogen, hydroxy, cyano, amino, or mono- or di(C₁-C₃ alkyl)amino; or
wherein R⁷ and R⁸ together with the nitrogen to which they are attached form ring Q, wherein ring Q is
a 3-7 membered monocyclic heterocyclyl group, and
the heterocyclyl group is fused to an aromatic or non-aromatic ring containing 3-6 ring members of which 1 or 2 are optionally nitrogen atoms and the remainder carbons, wherein
the fused heterocyclyl group is optionally substituted, on an aromatic or non-aromatic portion, with 1-4 R⁹ groups, wherein each R⁹ is independently C₁-C₆ alkyl, C₁-C₆ alkoxy, C₃-C₆ cycloalkyl, cyano, hydroxy, oxo, halogen, halo C₁-C₃ alkyl, hydroxy C₁-C₆ alkyl, amino, mono- or di-(C₁-C₆ alkyl) amino, or phenyl or phenyl(C₁-C₃ alkanoyl) where each phenyl is optionally substituted with C₁-C₃ alkyl, C₁-C₃ alkoxy, halogen, hydroxy, cyano, amino, or mono- or di(C₁-C₃ alkyl)amino; or
wherein R⁷ and R⁸ together with the nitrogen to which they are attached form a 3-7 membered monocyclic heterocyclyl group, and
the heterocyclyl group forms a spirocyclic ring system having up to 10 total ring members with a carbon atom from the monocyclic heterocyclyl group and additional carbon or hetero atoms, wherein
wherein the spirocyclic ring system is optionally substituted with 1-4 R⁹ groups, wherein each R⁹ is independently C₁-C₆ alkyl, C₁-C₆ alkoxy, C₃-C₆ cycloalkyl, cyano, hydroxy, oxo, halogen, halo C₁-C₃ alkyl, hydroxy C₁-C₆ alkyl, amino, mono- or di-(C₁-C₆ alkyl) amino, or phenyl or phenyl(C₁-C₃ alkanoyl) where each phenyl is optionally substituted with C₁-C₃ alkyl, C₁-C₃ alkoxy, halogen, hydroxy, cyano, amino, or mono- or di(C₁-C₃ alkyl)amino.

37. (canceled)

38. (canceled)

39. A compound according to claim 34, wherein R⁷ and R⁸ together with the nitrogen to which they are attached form a piperidinyl, piperazinyl or morpholinyl group, each of which is optionally substituted with 1-4 R⁹ groups, wherein each R⁹ is independently C₁-C₆ alkyl, C₁-C₆ alkoxy, C₃-C₆ cycloalkyl, cyano, hydroxy, oxo, halogen, halo C₁-C₃ alkyl, hydroxy C₁-C₆ alkyl, amino, mono- or di-(C₁-C₆ alkyl) amino, or phenyl or phenyl(C₁-C₃ alkanoyl) where each phenyl is optionally substituted with C₁-C₃ alkyl, C₁-C₃ alkoxy, halogen, hydroxy, cyano, amino, or mono- or di(C₁-C₃ alkyl)amino.

40. (canceled)

41. (canceled)

42. A compound according claim 1, wherein the compound is of formula (II) or (III) 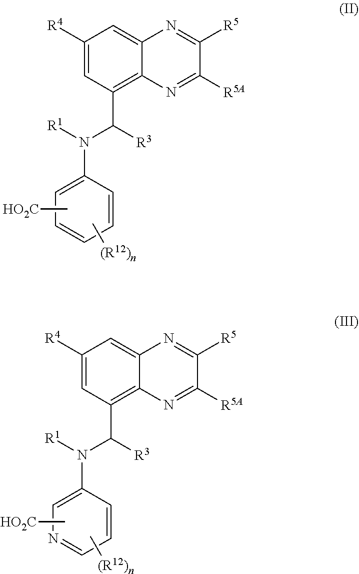
wherein n is 0, 1, 2, 3 or 4; and
m is 0, 1, 2 or 3.

43. (canceled)

44. (canceled)

45. A compound according to claim 42 or a pharmaceutically acceptable salt thereof, wherein:
R¹ is H, C₁-C₃ alkyl, or C₃-C₆ cycloalkyl;
each R¹² is independently C₁-C₄ alkyl; —OR^{A}, —C(O)OR^{A}, (C₁-C₃ alkyl)-OR^{A}, —C(O)N(R^{A})₂, cyano, or halogen;
each R^{A} is independently H, C₁-C₆ alkyl, or C₃-C₆ cycloalkyl; and
R³ is C₁-C₃ alkyl or C₃-C₆ cycloalkyl; and
R⁴ is H, C₁-C₃ alkyl, C₃-C₆ cycloalkyl, cyano, or halo, wherein each C₁-C₃ alkyl is optionally substituted with 1-5 halo groups.

46. A compound according to claim 42 wherein R³ is C₁-C₃ alkyl.

47. (canceled)

48. (canceled)

49. A compound according to claim 42 wherein R⁷ is hydrogen and R³ represents hydrogen, C₁-C₆ alkyl, C₃-C₆ cycloalkyl or C₃-C₆ cycloalkyl(C₁-C₃ alkyl).

50. (canceled)

51. A compound according to claim 42 wherein R^{5A} represents NR⁷R⁸ and R⁷ and R⁸ together with the nitrogen to which they are attached form ring Q, and ring Q is aziridinyl, azetidinyl, pyrrolidinyl, piperazinyl, morpholinyl, piperidinyl, or azepanyl, each of which is optionally substituted with 1-2 R⁹ groups, wherein each R⁹ is independently C₁-C₆ alkyl, C₁-C₆ alkoxy, C₃-C₆ cycloalkyl, cyano, hydroxy, oxo, halogen, halo C₁-C₃ alkyl, hydroxy C₁-C₆ alkyl, amino, mono- or di-(C₁-C₆ alkyl) amino, phenyl, 5-7 membered heterocyclyl, or 5-6 membered heteroaryl where each phenyl, 5-7 membered heterocyclyl and 5-6 membered heteroaryl is optionally substituted with one to four C₁-C₃ alkyl, C₁-C₃ alkoxy, halogen, hydroxy, cyano, amino, or mono- or di(C₁-C₃ alkyl)amino.

52. (canceled)

53. (canceled)

54. (canceled)

55. (canceled)

56. (canceled)

57. A compound according to claim 42, wherein R⁷ and R⁸ together with the nitrogen to which they are attached form a piperidinyl, piperazinyl or morpholinyl group, each of which is optionally substituted with 1-4 R⁹ groups, wherein each R⁹ is independently methyl, ethyl, methoxy, ethoxy, cyano, fluoro, chloro, trifluoromethyl, cyclopropyl, or cyclopropylmethyl.

58. A compound of Formula (IV): 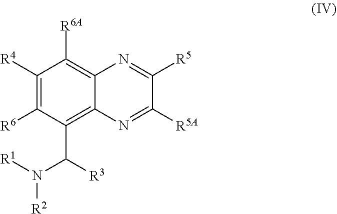
or a pharmaceutically acceptable salt, prodrug, solvate, hydrate, isomer, deuterated form, or tautomer thereof, wherein:
R¹ is H, C₁-C₃ alkyl, or C₃-C₆ cycloalkyl;
R² is phenyl or a 5-6 membered heteroaryl group, wherein each phenyl and heteroaryl is optionally substituted with 1-5 R¹²;
each R¹² is independently C₁-C₄ alkyl, —OR^{A}, —C(O)OR^{A}, (C₁-C₃ alkyl)-OR^{A}, —C(O)N(R^{A})₂, cyano, or halogen;
each R^{A} is independently H, C₁-C₆ alkyl, or C₃-C₆ cycloalkyl; and
R³ is C₁-C₃ alkyl optionally substituted, polysubstituted or persubstituted with fluoro, C₃-C₆ cycloalkyl, C₁-C₃ alkenyl, C₂-C₃ alkynyl, or C₅-C₆ cycloalkenyl;
R⁴ is H, halogen, C₁-C₃ alkyl, C₃-C₆ cycloalkyl, C₁-C₃ alkenyl, C₂-C₃ alkynyl, or C₅-C₆ cycloalkenyl or cyano, wherein each C₁-C₃ alkyl and C₃-C₆ cycloalkyl is optionally substituted with 1-5 halo groups;
R⁶ and R^{6A} independently represent H or C₁-C₆ alkyl;
R⁵ is C₁-C₂ alkyl, cyclopropyl, bromo, chloro, fluoro, cyano, or trifluoromethyl;
R^{5A} represents NR⁷R⁸, C₁-C₆ alkyl, C₃-C₆ cycloalkyl, C₃-C₆ cycloalkyl(C₁-C₃ alkyl), phenyl or 5-6 membered heteroaryl, wherein each C₁-C₆ alkyl, C₃-C₆ cycloalkyl, C₃-C₆ cycloalkyl(C₁-C₃ alkyl), phenyl and heteroaryl is optionally substituted with 1-3 of halogen, halo(C₁-C₃ alkyl), hydroxy, C₁-C₃ alkyl, C₁-C₃ alkoxy, cyano, amino, or mono- or di(C₁-C₃) alkyl amino;
R⁷ represents H or C₁-C₃ alkyl;
R³ represent H, C₁-C₆ alkyl, C₃-C₆ cycloalkyl or C₃-C₆ cycloalkyl(C₁-C₃ alkyl) wherein each C₁-C₆ alkyl, C₃-C₆ cycloalkyl and C₃-C₆ cycloalkyl(C₁-C₃ alkyl) is optionally substituted with 1-3 of halogen, halo(C₁-C₃ alkyl), hydroxy, C₁-C₃ alkyl, C₁-C₃ alkoxy, cyano, amino, or mono- or di(C₁-C₃) alkyl amino; or
R⁷ and R⁸ together with the nitrogen to which they are attached form ring Q, wherein ring Q is
a 3-8 membered monocyclic heterocyclyl group, wherein the heterocyclyl group
is optionally fused to an aromatic or non-aromatic ring containing 3-6 ring members of which 1 or 2 are optionally nitrogen, oxygen or sulfur atoms and the remainder carbons, or
with a carbon atom from the monocyclic heterocyclyl group and additional carbon or hetero atoms, forms a spirocyclic ring system having up to 10 total ring members, wherein the ring members are carbons or 1 or 2 hetero atoms selected from nitrogen, sulfur and oxygen atoms, and
wherein each ring Q is optionally substituted, on an aromatic or non-aromatic portion, with 1-4 R⁹ groups, wherein each R⁹ is independently (i) C₁-C₆ alkyl, (ii) C₁-C₆ alkoxy, (iii) C₃-C₆ cycloalkyl, (iv) cyano, (ii) hydroxy, (v) oxo, (vi) halogen, (vii) halo C₁-C₃ alkyl, (viii) hydroxy C₁-C₆ alkyl, (ix) amino, (x) mono- or di-(C₁-C₆ alkyl) amino, (xi) C₂-C₆ alkanoyl optionally substituted with 1 or 2 of C₁-C₃ alkyl, C₁-C₃ alkoxy, C₃-C₆ cycloalkyl, cyano, hydroxy, oxo, halogen, halo C₁-C₃ alkyl, hydroxy C₁-C₃ alkyl, amino, or mono- or di-(C₁-C₆ alkyl) amino, wherein each C₁-C₃ alkyl or C₃-C₆ cycloalkyl on the alkanoyl is optionally substituted with halogen, hydroxy or amino, or (xii) C₃-C₆ cycloalkyloxy, or (xiii) phenyl, phenyl(C₁-C₃ alkanoyl), 5-7 membered heterocyclyl, or 5-6 membered heteroaryl where each phenyl, 5-7 membered heterocyclyl and 5-6 membered heteroaryl is optionally substituted with one to four C₁-C₃ alkyl, C₁-C₃ alkoxy, halogen, hydroxy, cyano, oxo, amino, or mono- or di(C₁-C₃ alkyl)amino; or.
a compound of Formula (IV-A): 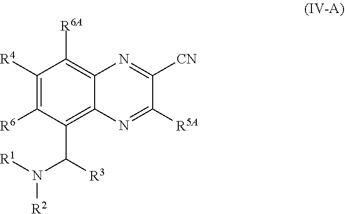
or a Pharmaceutically acceptable salt, prodrug, solvate, hydrate, isomer, deuterated form, or tautomer thereof, wherein:
R¹ is H, C₁-C₃ alkyl, or C₃-C₆ cycloalkyl;
R² is phenyl or a 5-6 membered heteroaryl group, wherein each phenyl and heteroaryl is optionally substituted with 1-5 R¹²;
each R¹² is independently C₁-C₄ alkyl, —OR^{A}, —C(O)OR^{A}, (C₁-C₃ alkyl)-OR^{A}, —C(O)N(R^{A})₂, cyano, or halogen;
each R^{A} is independently H, C₁-C₆ alkyl, or C₃-C₆ cycloalkyl; and
R³ is C₁-C₃ alkyl optionally substituted, polysubstituted or persubstituted with fluoro, C₃-C₆ cycloalkyl, C₁-C₃ alkenyl, C₂-C₃ alkynyl, or C₅-C₆ cycloalkenyl;
R⁴ is H, halogen, C₁-C₃ alkyl, C₃-C₆ cycloalkyl, C₁-C₃ alkenyl, C₂-C₃ alkynyl, or C₅-C₆ cycloalkenyl or cyano, wherein each C₁-C₃ alkyl and C₃-C₆ cycloalkyl is optionally substituted with 1-5 halo groups;
R⁶ and R^{6A} independently represent H or C₁-C₆ alkyl;
R^{5A} represents NR⁷R⁸, C₁-C₆ alkyl, C₃-C₆ cycloalkyl, C₃-C₆ cycloalkyl(C₁-C₃ alkyl), phenyl or 5-6 membered heteroaryl, wherein each C₁-C₆ alkyl, C₃-C₆ cycloalkyl, C₃-C₆ cycloalkyl(C₁-C₃ alkyl), phenyl and heteroaryl is optionally substituted with 1-3 of halogen, halo(C₁-C₃ alkyl), hydroxy, C₁-C₃ alkyl, C₁-C₃ alkoxy, cyano, amino, or mono- or di(C₁-C₃) alkyl amino;
R⁷ represents H or C₁-C₃ alkyl;
R⁸ represent H, C₁-C₆ alkyl, C₃-C₆ cycloalkyl or C₃-C₆ cycloalkyl(C₁-C₃ alkyl) wherein each C₁-C₆ alkyl, C₃-C₆ cycloalkyl and C₃-C₆ cycloalkyl(C₁-C₃ alkyl) is optionally substituted with 1-3 of halogen, halo(C₁-C₃ alkyl), hydroxy, C₁-C₃ alkyl, C₁-C₃ alkoxy, cyano, amino, or mono- or di(C₁-C₃) alkyl amino; or
R⁷ and R⁸ together with the nitrogen to which they are attached form ring Q, wherein ring Q is
a 3-8 membered monocyclic heterocyclyl group, wherein the heterocyclyl group
is optionally fused to an aromatic or non-aromatic ring containing 3-6 ring members of which 1 or 2 are optionally nitrogen, oxygen or sulfur atoms and the remainder carbons, or
with a carbon atom from the monocyclic heterocyclyl group and additional carbon or hetero atoms, forms a spirocyclic ring system having up to 10 total ring members, wherein the ring members are carbons or 1 or 2 hetero atoms selected from nitrogen, sulfur and oxygen atoms, and
wherein each ring Q is optionally substituted, on an aromatic or non-aromatic portion, with 1-4 R⁹ groups, wherein each R⁹ is independently (i) C₁-C₆ alkyl, (ii) C₁-C₆ alkoxy, (iii) C₃-C₆ cycloalkyl, (iv) cyano, (ii) hydroxy, (v) oxo, (vi) halogen, (vii) halo C₁-C₃ alkyl, (viii) hydroxy C₁-C₆ alkyl, (ix) amino, (x) mono- or di-(C₁-C₆ alkyl) amino, (xi) C₂-C₆ alkanoyl optionally substituted with 1 or 2 of C₁-C₃ alkyl, C₁-C₃ alkoxy, C₃-C₆ cycloalkyl, cyano, hydroxy, oxo, halogen, halo C₁-C₃ alkyl, hydroxy C₁-C₃ alkyl, amino, or mono- or di-(C₁-C₆ alkyl) amino, wherein each C₁-C₃ alkyl or C₃-C₆ cycloalkyl on the alkanoyl is optionally substituted with halogen, hydroxy or amino, or (xii) C₃-C₆ cycloalkyloxy, or (xiii) phenyl, phenyl(C₁-C₃ alkanoyl), 5-7 membered heterocyclyl, or 5-6 membered heteroaryl where each phenyl, 5-7 membered heterocyclyl and 5-6 membered heteroaryl is optionally substituted with one to four C₁-C₃ alkyl, C₁-C₃ alkoxy, halogen, hydroxy, cyano, oxo, amino, or mono- or di(C₁-C₃ alkyl)amino; or
a compound of Formula (IV-B): 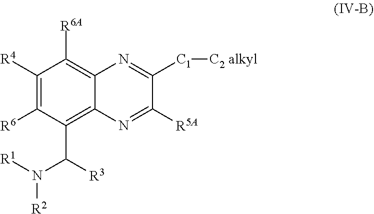
or a pharmaceutically acceptable salt, prodrug, solvate, hydrate, isomer, deuterated form, or tautomer thereof, wherein:
R¹ is H, C₁-C₃ alkyl, or C₃-C₆ cycloalkyl;
R² is phenyl or a 5-6 membered heteroaryl group, wherein each phenyl and heteroaryl is optionally substituted with 1-5 R¹²;
each R¹² is independently C₁-C₄ alkyl, —OR^{A}, —C(O)OR^{A}, (C₁-C₃ alkyl)-OR^{A}, —C(O)N(R^{A})₂, cyano, or halogen;
each R^{A} is independently H, C₁-C₆ alkyl, or C₃-C₆ cycloalkyl; and
R³ is C₁-C₃ alkyl optionally substituted, polysubstituted or persubstituted with fluoro, C₃-C₆ cycloalkyl, C₁-C₃ alkenyl, C₂-C₃ alkynyl, or C₅-C₆ cycloalkenyl;
R⁴ is H, halogen, C₁-C₃ alkyl, C₃-C₆ cycloalkyl, C₁-C₃ alkenyl, C₂-C₃ alkynyl, or C₅-C₆ cycloalkenyl or cyano, wherein each C₁-C₃ alkyl and C₃-C₆ cycloalkyl is optionally substituted with 1-5 halo groups;
R⁶ and R^{6A} independently represent H or C₁-C₆ alkyl;
R^{5A} represents NR⁷R⁸, C₁-C₆ alkyl, C₃-C₆ cycloalkyl, C₃-C₆ cycloalkyl(C₁-C₃ alkyl), phenyl or 5-6 membered heteroaryl, wherein each C₁-C₆ alkyl, C₃-C₆ cycloalkyl, C₃-C₆ cycloalkyl(C₁-C₃ alkyl), phenyl and heteroaryl is optionally substituted with 1-3 of halogen, halo(C₁-C₃ alkyl), hydroxy, C₁-C₃ alkyl, C₁-C₃ alkoxy, cyano, amino, or mono- or di(C₁-C₃) alkyl amino;
R⁷ represents H or C₁-C₃ alkyl;
R⁸ represent H, C₁-C₆ alkyl, C₃-C₆ cycloalkyl or C₃-C₆ cycloalkyl(C₁-C₃ alkyl) wherein each C₁-C₆ alkyl, C₃-C₆ cycloalkyl and C₃-C₆ cycloalkyl(C₁-C₃ alkyl) is optionally substituted with 1-3 of halogen, halo(C₁-C₃ alkyl), hydroxy, C₁-C₃ alkyl, C₁-C₃ alkoxy, cyano, amino, or mono- or di(C₁-C₃) alkyl amino; or
R⁷ and R⁸ together with the nitrogen to which they are attached form ring Q, wherein ring Q is
a 3-8 membered monocyclic heterocyclyl group, wherein the heterocyclyl group
is optionally fused to an aromatic or non-aromatic ring containing 3-6 ring members of which 1 or 2 are optionally nitrogen, oxygen or sulfur atoms and the remainder carbons, or
with a carbon atom from the monocyclic heterocyclyl group and additional carbon or hetero atoms, forms a spirocyclic ring system having up to 10 total ring members, wherein the ring members are carbons or 1 or 2 hetero atoms selected from nitrogen, sulfur and oxygen atoms, and
wherein each ring Q is optionally substituted, on an aromatic or non-aromatic portion, with 1-4 R⁹ groups, wherein each R⁹ is independently (i) C₁-C₆ alkyl, (ii) C₁-C₆ alkoxy, (iii) C₃-C₆ cycloalkyl, (iv) cyano, (ii) hydroxy, (v) oxo, (vi) halogen, (vii) halo C₁-C₃ alkyl, (viii) hydroxy C₁-C₆ alkyl, (ix) amino, (x) mono- or di-(C₁-C₆ alkyl) amino, (xi) C₂-C₆ alkanoyl optionally substituted with 1 or 2 of C₁-C₃ alkyl, C₁-C₃ alkoxy, C₃-C₆ cycloalkyl, cyano, hydroxy, oxo, halogen, halo C₁-C₃ alkyl, hydroxy C₁-C₃ alkyl, amino, or mono- or di-(C₁-C₆ alkyl) amino, wherein each C₁-C₃ alkyl or C₃-C₆ cycloalkyl on the alkanoyl is optionally substituted with halogen, hydroxy or amino, or (xii) C₃-C₆ cycloalkyloxy, or (xiii) phenyl, phenyl(C₁-C₃ alkanoyl), 5-7 membered heterocyclyl, or 5-6 membered heteroaryl where each phenyl, 5-7 membered heterocyclyl and 5-6 membered heteroaryl is optionally substituted with one to four C₁-C₃ alkyl, C₁-C₃ alkoxy, halogen, hydroxy, cyano, oxo, amino, or mono- or di(C₁-C₃ alkyl)amino; or
a compound of Formula (IV-C): 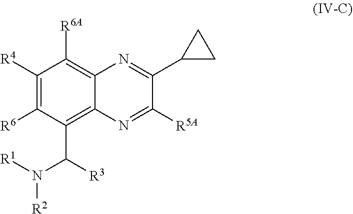
or a Pharmaceutically acceptable salt, prodrug, solvate, hydrate, isomer, deuterated form, or tautomer thereof, wherein:
R¹ is H, C₁-C₃ alkyl, or C₃-C₆ cycloalkyl;
R² is phenyl or a 5-6 membered heteroaryl group, wherein each phenyl and heteroaryl is optionally substituted with 1-5 R¹²;
each R¹² is independently C₁-C₄ alkyl, —OR^{A}, —C(O)OR^{A}, (C₁-C₃ alkyl)-OR^{A}, —C(O)N(R^{A})₂, cyano, or halogen;
each R^{A} is independently H, C₁-C₆ alkyl, or C₃-C₆ cycloalkyl; and
R³ is C₁-C₃ alkyl optionally substituted, polysubstituted or persubstituted with fluoro, C₃-C₆ cycloalkyl, C₁-C₃ alkenyl, C₂-C₃ alkynyl, or C₅-C₆ cycloalkenyl;
R⁴ is H, halogen, C₁-C₃ alkyl, C₃-C₆ cycloalkyl, C₁-C₃ alkenyl, C₂-C₃ alkynyl, or C₅-C₆ cycloalkenyl or cyano, wherein each C₁-C₃ alkyl and C₃-C₆ cycloalkyl is optionally substituted with 1-5 halo groups;
R⁶ and R^{6A} independently represent H or C₁-C₆ alkyl;
R^{5A} represents NR⁷R⁸, C₁-C₆ alkyl, C₃-C₆ cycloalkyl, C₃-C₆ cycloalkyl(C₁-C₃ alkyl), phenyl or 5-6 membered heteroaryl, wherein each C₁-C₆ alkyl, C₃-C₆ cycloalkyl, C₃-C₆ cycloalkyl(C₁-C₃ alkyl), phenyl and heteroaryl is optionally substituted with 1-3 of halogen, halo(C₁-C₃ alkyl), hydroxy, C₁-C₃ alkyl, C₁-C₃ alkoxy, cyano, amino, or mono- or di(C₁-C₃) alkyl amino;
R⁷ represents H or C₁-C₃ alkyl;
R⁸ represent H, C₁-C₆ alkyl, C₃-C₆ cycloalkyl or C₃-C₆ cycloalkyl(C₁-C₃ alkyl) wherein each C₁-C₆ alkyl, C₃-C₆ cycloalkyl and C₃-C₆ cycloalkyl(C₁-C₃ alkyl) is optionally substituted with 1-3 of halogen, halo(C₁-C₃ alkyl), hydroxy, C₁-C₃ alkyl, C₁-C₃ alkoxy, cyano, amino, or mono- or di(C₁-C₃) alkyl amino; or
R⁷ and R⁸ together with the nitrogen to which they are attached form ring Q, wherein ring Q is
a 3-8 membered monocyclic heterocyclyl group, wherein the heterocyclyl group
is optionally fused to an aromatic or non-aromatic ring containing 3-6 ring members of which 1 or 2 are optionally nitrogen, oxygen or sulfur atoms and the remainder carbons, or
with a carbon atom from the monocyclic heterocyclyl group and additional carbon or hetero atoms, forms a spirocyclic ring system having up to 10 total ring members, wherein the ring members are carbons or 1 or 2 hetero atoms selected from nitrogen, sulfur and oxygen atoms, and
wherein each ring Q is optionally substituted, on an aromatic or non-aromatic portion, with 1-4 R⁹ groups, wherein each R⁹ is independently (i) C₁-C₆ alkyl, (ii) C₁-C₆ alkoxy, (iii) C₃-C₆ cycloalkyl, (iv) cyano, (ii) hydroxy, (v) oxo, (vi) halogen, (vii) halo C₁-C₃ alkyl, (viii) hydroxy C₁-C₆ alkyl, (ix) amino, (x) mono- or di-(C₁-C₆ alkyl) amino, (xi) C₂-C₆ alkanoyl optionally substituted with 1 or 2 of C₁-C₃ alkyl, C₁-C₃ alkoxy, C₃-C₆ cycloalkyl, cyano, hydroxy, oxo, halogen, halo C₁-C₃ alkyl, hydroxy C₁-C₃ alkyl, amino, or mono- or di-(C₁-C₆ alkyl) amino, wherein each C₁-C₃ alkyl or C₃-C₆ cycloalkyl on the alkanoyl is optionally substituted with halogen, hydroxy or amino, or (xii) C₃-C₆ cycloalkyloxy, or (xiii) phenyl, phenyl(C₁-C₃ alkanoyl), 5-7 membered heterocyclyl, or 5-6 membered heteroaryl where each phenyl, 5-7 membered heterocyclyl and 5-6 membered heteroaryl is optionally substituted with one to four C₁-C₃ alkyl, C₁-C₃ alkoxy, halogen, hydroxy, cyano, oxo, amino, or mono- or di(C₁-C₃ alkyl)amino; or
a compound of Formula (IV-D): 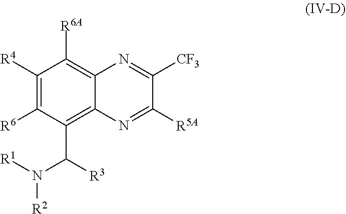
or a Pharmaceutically acceptable salt, prodrug, solvate, hydrate, isomer, deuterated form, or tautomer thereof, wherein:
R¹ is H, C₁-C₃ alkyl, or C₃-C₆ cycloalkyl;
R² is phenyl or a 5-6 membered heteroaryl group, wherein each phenyl and heteroaryl is optionally substituted with 1-5 R¹²:
each R¹² is independently C₁-C₄ alkyl, —OR^{A}, —C(O)OR^{A}, (C₁-C₃ alkyl)-OR^{A}, —C(O)N(R^{A})₂, cyano, or halogen;
each R^{A} is independently H, C₁-C₆ alkyl, or C₃-C₆ cycloalkyl; and
R³ is C₁-C₃ alkyl optionally substituted, polysubstituted or persubstituted with fluoro, C₃-C₆ cycloalkyl, C₁-C₃ alkenyl, C₂-C₃ alkynyl, or C₅-C₆ cycloalkenyl;
R⁴ is H, halogen, C₁-C₃ alkyl, C₃-C₆ cycloalkyl, C₁-C₃ alkenyl, C₂-C₃ alkynyl, or C₅-C₆ cycloalkenyl or cyano, wherein each C₁-C₃ alkyl and C₃-C₆ cycloalkyl is optionally substituted with 1-5 halo groups;
R⁶ and R^{6A} independently represent H or C₁-C₆ alkyl;
R^{5A} represents NR⁷R⁸, C₁-C₆ alkyl, C₃-C₆ cycloalkyl, C₃-C₆ cycloalkyl(C₁-C₃ alkyl), phenyl or 5-6 membered heteroaryl, wherein each C₁-C₆ alkyl, C₃-C₆ cycloalkyl, C₃-C₆ cycloalkyl(C₁-C₃ alkyl), phenyl and heteroaryl is optionally substituted with 1-3 of halogen, halo(C₁-C₃ alkyl), hydroxy, C₁-C₃ alkyl, C₁-C₃ alkoxy, cyano, amino, or mono- or di(C₁-C₃) alkyl amino;
R⁷ represents H or C₁-C₃ alkyl;
R⁸ represent H, C₁-C₆ alkyl, C₃-C₆ cycloalkyl or C₃-C₆ cycloalkyl(C₁-C₃ alkyl) wherein each C₁-C₆ alkyl, C₃-C₆ cycloalkyl and C₃-C₆ cycloalkyl(C₁-C₃ alkyl) is optionally substituted with 1-3 of halogen, halo(C₁-C₃ alkyl), hydroxy, C₁-C₃ alkyl, C₁-C₃ alkoxy, cyano, amino, or mono- or di(C₁-C₃) alkyl amino; or
R⁷ and R⁸ together with the nitrogen to which they are attached form ring Q, wherein ring Q is
a 3-8 membered monocyclic heterocyclyl group, wherein the heterocyclyl group
is optionally fused to an aromatic or non-aromatic ring containing 3-6 ring members of which 1 or 2 are optionally nitrogen, oxygen or sulfur atoms and the remainder carbons, or
with a carbon atom from the monocyclic heterocyclyl group and additional carbon or hetero atoms, forms a spirocyclic ring system having up to 10 total ring members, wherein the ring members are carbons or 1 or 2 hetero atoms selected from nitrogen, sulfur and oxygen atoms, and
wherein each ring Q is optionally substituted, on an aromatic or non-aromatic portion, with 1-4 R⁹ groups, wherein each R⁹ is independently (i) C₁-C₆ alkyl, (ii) C₁-C₆ alkoxy, (iii) C₃-C₆ cycloalkyl, (iv) cyano, (ii) hydroxy, (v) oxo, (vi) halogen, (vii) halo C₁-C₃ alkyl, (viii) hydroxy C₁-C₆ alkyl, (ix) amino, (x) mono- or di-(C₁-C₆ alkyl) amino, (xi) C₂-C₆ alkanoyl optionally substituted with 1 or 2 of C₁-C₃ alkyl, C₁-C₃ alkoxy, C₃-C₆ cycloalkyl, cyano, hydroxy, oxo, halogen, halo C₁-C₃ alkyl, hydroxy C₁-C₃ alkyl, amino, or mono- or di-(C₁-C₆ alkyl) amino, wherein each C₁-C₃ alkyl or C₃-C₆ cycloalkyl on the alkanoyl is optionally substituted with halogen, hydroxy or amino, or (xii) C₃-C₆ cycloalkyloxy, or (xiii) phenyl, phenyl(C₁-C₃ alkanoyl), 5-7 membered heterocyclyl, or 5-6 membered heteroaryl where each phenyl, 5-7 membered heterocyclyl and 5-6 membered heteroaryl is optionally substituted with one to four C₁-C₃ alkyl, C₁-C₃ alkoxy, halogen, hydroxy, cyano, oxo, amino, or mono- or di(C₁-C₃ alkyl)amino; or
a compound of Formula (IV-E): 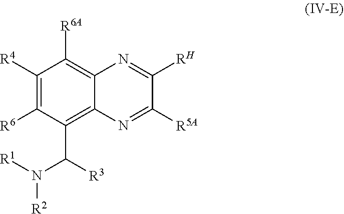
or a pharmaceutically acceptable salt, prodrug, solvate, hydrate, isomer, deuterated form, or tautomer thereof, wherein:
R¹ is H, C₁-C₃ alkyl, or C₃-C₆ cycloalkyl;
R² is phenyl or a 5-6 membered heteroaryl group, wherein each phenyl and heteroaryl is optionally substituted with 1-5 R¹²;
each R¹² is independently C₁-C₄ alkyl, —OR^{A}, —C(O)OR^{A}, (C₁-C₃ alkyl)-OR^{A}, —C(O)N(R^{A})₂, cyano, or halogen;
each R^{A} is independently H, C₁-C₆ alkyl, or C₃-C₆ cycloalkyl; and
R³ is C₁-C₃ alkyl optionally substituted, polysubstituted or persubstituted with fluoro, C₃-C₆ cycloalkyl, C₁-C₃ alkenyl, C₂-C₃ alkynyl, or C₅-C₆ cycloalkenyl;
R⁴ is H, halogen, C₁-C₃ alkyl, C₃-C₆ cycloalkyl, C₁-C₃ alkenyl, C₂-C₃ alkynyl, or C₅-C₆ cycloalkenyl or cyano, wherein each C₁-C₃ alkyl and C₃-C₆ cycloalkyl is optionally substituted with 1-5 halo groups;
R^{H} is bromo, chloro or fluoro,
R⁶ and R^{6A} independently represent H or C₁-C₆ alkyl;
R^{5A} represents NR⁷R⁸, C₁-C₆ alkyl, C₃-C₆ cycloalkyl, C₃-C₆ cycloalkyl(C₁-C₃ alkyl), phenyl or 5-6 membered heteroaryl, wherein each C₁-C₆ alkyl, C₃-C₆ cycloalkyl, C₃-C₆ cycloalkyl(C₁-C₃ alkyl), phenyl and heteroaryl is optionally substituted with 1-3 of halogen, halo(C₁-C₃ alkyl), hydroxy, C₁-C₃ alkyl, C₁-C₃ alkoxy, cyano, amino, or mono- or di(C₁-C₃) alkyl amino;
R⁷ represents H or C₁-C₃ alkyl;
R⁸ represent H, C₁-C₆ alkyl, C₃-C₆ cycloalkyl or C₃-C₆ cycloalkyl(C₁-C₃ alkyl) wherein each C₁-C₆ alkyl, C₃-C₆ cycloalkyl and C₃-C₆ cycloalkyl(C₁-C₃ alkyl) is optionally substituted with 1-3 of halogen, halo(C₁-C₃ alkyl), hydroxy, C₁-C₃ alkyl, C₁-C₃ alkoxy, cyano, amino, or mono- or di(C₁-C₃) alkyl amino; or
R⁷ and R⁸ together with the nitrogen to which they are attached form ring Q, wherein ring Q is
a 3-8 membered monocyclic heterocyclyl group, wherein the heterocyclyl group
is optionally fused to an aromatic or non-aromatic ring containing 3-6 ring members of which 1 or 2 are optionally nitrogen, oxygen or sulfur atoms and the remainder carbons, or
with a carbon atom from the monocyclic heterocyclyl group and additional carbon or hetero atoms, forms a spirocyclic ring system having up to 10 total ring members, wherein the ring members are carbons or 1 or 2 hetero atoms selected from nitrogen, sulfur and oxygen atoms, and
wherein each ring Q is optionally substituted, on an aromatic or non-aromatic portion, with 1-4 R⁹ groups, wherein each R⁹ is independently (i) C₁-C₆ alkyl, (ii) C₁-C₆ alkoxy, (iii) C₃-C₆ cycloalkyl, (iv) cyano, (ii) hydroxy, (v) oxo, (vi) halogen, (vii) halo C₁-C₃ alkyl, (viii) hydroxy C₁-C₆ alkyl, (ix) amino, (x) mono- or di-(C₁-C₆ alkyl) amino, (xi) C₂-C₆ alkanoyl optionally substituted with 1 or 2 of C₁-C₃ alkyl, C₁-C₃ alkoxy, C₃-C₆ cycloalkyl, cyano, hydroxy, oxo, halogen, halo C₁-C₃ alkyl, hydroxy C₁-C₃ alkyl, amino, or mono- or di-(C₁-C₆ alkyl) amino, wherein each C₁-C₃ alkyl or C₃-C₆ cycloalkyl on the alkanoyl is optionally substituted with halogen, hydroxy or amino, or (xii) C₃-C₆ cycloalkyloxy, or (xiii) phenyl, phenyl(C₁-C₃ alkanoyl), 5-7 membered heterocyclyl, or 5-6 membered heteroaryl where each phenyl, 5-7 membered heterocyclyl and 5-6 membered heteroaryl is optionally substituted with one to four C₁-C₃ alkyl, C₁-C₃ alkoxy, halogen, hydroxy, cyano, oxo, amino, or mono- or di(C₁-C₃ alkyl)amino.

59. (canceled)

60. (canceled)

61. (canceled)

62. (canceled)

63. (canceled)

64. A compound according to claim 1, which is:
2-((1-(2-cyano-7-methyl-3-(piperidin-1-yl)quinoxalin-5-yl)ethyl)amino)benzoic acid;
2-((1-(2-cyano-3-(isoindolin-2-yl)-7-methylquinoxalin-5-yl)ethyl)amino)benzoic acid;
2-((1-(2-cyano-3-(4,4-difluoropiperidin-1-yl)-7-methylquinoxalin-5-yl)ethyl)amino)benzoic acid;
6-chloro-3-((1-(2-cyano-3-(4,4-difluoropiperidin-1-yl)-7-methylquinoxalin-5-yl)ethyl)amino)picolinic acid;
2-((1-(2-cyano-3-(isoindolin-2-yl)-7-methylquinoxalin-5-yl)ethyl)amino)benzoic acid;
6-chloro-3-((1-(2-cyano-3-(6,6-difluoro-3-azabicyclo[3.1.1]heptan-3-yl)-7-methylquinoxalin-5-yl)ethyl)amino)picolinic acid;
6-chloro-3-((1-(2-cyano-3-(6,6-difluoro-2-azabicyclo[2.2.1]heptan-2-yl)-7-methylquinoxalin-5-yl)ethyl)amino)picolinic acid;
6-chloro-3-((1-(2-cyano-3-(3,3-difluoropiperidin-1-yl)-7-methylquinoxalin-5-yl)ethyl)amino)picolinic acid;
6-chloro-3-((1-(2-cyano-3-(((2,2-difluorocyclopropyl)methyl)amino)-7-methylquinoxalin-5-yl)ethyl)amino)picolinic acid;
6-chloro-3-((1-(2-cyano-7-methyl-3-(((1-(trifluoromethyl)cyclopropyl)methyl)amino)quinoxalin-5-yl)ethyl)amino)picolinic acid;
6-chloro-3-((1-(2-cyano-3-(1,1-difluoro-5-azaspiro[2.3]hexan-5-yl)-7-methylquinoxalin-5-yl)ethyl)amino)picolinic acid;
3-((1-(2-cyano-3-(4,4-difluoropiperidin-1-yl)-7-methylquinoxalin-5-yl)ethyl)amino)-6-methylpicolinic acid;
2-((1-(2-cyano-3-((cyclopropylmethyl)amino)-7-methylquinoxalin-5-yl)ethyl)amino)benzoic acid;
6-chloro-3-((1-(2-cyano-7-methyl-3-(3-(trifluoromethyl)piperidin-1-yl)quinoxalin-5-yl)ethyl)amino)picolinic acid;
6-chloro-3-((1-(2-cyano-3-(4,4-difluoropiperidin-1-yl)-7-(trifluoromethyl)quinoxalin-5-yl)ethyl)amino)picolinic acid;
6-chloro-3-((1-(2-cyano-3-(6,6-difluoro-3-azabicyclo[3.1.0]hexan-3-yl)-7-methylquinoxalin-5-yl)ethyl)amino)picolinic acid;
6-chloro-3-((1-(2-cyano-3-(4,4-difluoro-6-azaspiro[2.5]octan-6-yl)-7-methylquinoxalin-5-yl)ethyl)amino)picolinic acid;
6-chloro-3-((1-(2-cyano-3-(((1-fluorocyclopropyl)methyl)amino)-7-methylquinoxalin-5-yl)ethyl)amino)picolinic acid;
6-chloro-3-((1-(2-cyano-3-(5-fluoroisoindolin-2-yl)-7-methylquinoxalin-5-yl)ethyl)amino)picolinic acid;
6-chloro-3-((1-(2-cyano-3-((cyclopropylmethyl)amino)-7-methylquinoxalin-5-yl)ethyl)amino)picolinic acid;
6-chloro-3-((1-(2-cyano-3-(4,4-difluoropiperidin-1-yl)-7-fluoroquinoxalin-5-yl)ethyl)amino)picolinic acid;
2-((1-(2-cyano-3-(4,4-difluoropiperidin-1-yl)-7-fluoroquinoxalin-5-yl)ethyl)amino)benzoic acid;
2-((1-(2-cyano-7-fluoro-3-(4-fluoropiperidin-1-yl)quinoxalin-5-yl)ethyl)amino)benzoic acid;
2-((1-(2,7-dicyano-3-(4-fluoropiperidin-1-yl)quinoxalin-5-yl)ethyl)amino)benzoic acid;
2-((1-(2,7-dicyano-3-(4,4-difluoropiperidin-1-yl)quinoxalin-5-yl)ethyl)amino)benzoic acid;
2-((1-(7-chloro-2-cyano-3-(4,4-difluoropiperidin-1-yl)quinoxalin-5-yl)ethyl)amino)benzoic acid;
6-chloro-3-((1-(2-cyano-7-cyclopropyl-3-(4,4-difluoropiperidin-1-yl)quinoxalin-5-yl)ethyl)amino)picolinic acid;
6-chloro-3-((1-(7-chloro-2-cyano-3-(4,4-difluoropiperidin-1-yl)quinoxalin-5-yl)ethyl)amino)picolinic acid;
6-chloro-3-((1-(3-(4,4-difluoropiperidin-1-yl)-2,7-dimethylquinoxalin-5-yl)ethyl)amino)picolinic acid;
2-((1-(3-(4,4-difluoropiperidin-1-yl)-2,7-dimethylquinoxalin-5-yl)ethyl)amino)benzoic acid;
2-((1-(2-cyano-7-cyclopropyl-3-(4,4-difluoropiperidin-1-yl)quinoxalin-5-yl)ethyl)amino)benzoic acid;
2-((1-(2-cyano-3-(4,4-difluoropiperidin-1-yl)-7-methoxyquinoxalin-5-yl)ethyl)amino)benzoic acid;
5-((1-(2-cyano-3-(4,4-difluoropiperidin-1-yl)-7-methylquinoxalin-5-yl)ethyl)amino)thiazole-4-carboxylic acid;
2-chloro-5-((1-(2-cyano-3-(4,4-difluoropiperidin-1-yl)-7-methylquinoxalin-5-yl)ethyl)amino)thiazole-4-carboxylic acid;
2-((1-(2-cyano-3-(4,4-difluoropiperidin-1-yl)-7-methylquinoxalin-5-yl)ethyl)amino)thiophene-3-carboxylic acid;
3-((1-(2-cyano-3-(4,4-difluoropiperidin-1-yl)-7-methylquinoxalin-5-yl)ethyl)amino)thiophene-2-carboxylic acid;
6-chloro-3-((1-(2-cyano-7-methyl-3-(1-(trifluoromethyl)-3-azabicyclo[3.1.0]hexan-3-yl)quinoxalin-5-yl)ethyl)amino)picolinic acid;
6-chloro-3-((1-(2-cyano-3-cyclopropyl-7-methylquinoxalin-5-yl)ethyl)amino)picolinic acid;
6-chloro-3-((1-(2-cyano-3-(3-(difluoromethyl)piperidin-1-yl)-7-methylquinoxalin-5-yl)ethyl)amino)picolinic acid;
6-chloro-3-((1-(2-cyano-3-(1-(fluoromethyl)-3-azabicyclo[3.1.0]hexan-3-yl)-7-methylquinoxalin-5-yl)ethyl)amino)picolinic acid;
6-chloro-3-((1-(2-cyclopropyl-3-(4,4-difluoropiperidin-1-yl)-7-methylquinoxalin-5-yl)ethyl)amino)picolinic acid;
6-chloro-3-((1-(3-(4,4-difluoropiperidin-1-yl)-7-methyl-2-(trifluoromethyl)quinoxalin-5-yl)ethyl)amino)picolinic acid;
2-((1-(3-(4,4-difluoropiperidin-1-yl)-7-methyl-2-(trifluoromethyl)quinoxalin-5-yl)ethyl)amino)benzoic acid;
2-((1-(2-cyano-3-(4,4-difluoropiperidin-1-yl)-7-ethylquinoxalin-5-yl)ethyl)amino)benzoic acid;
6-chloro-3-((1-(2-chloro-3-(4,4-difluoropiperidin-1-yl)-7-methylquinoxalin-5-yl)ethyl)amino)picolinic acid;
2-((1-(2-chloro-3-(4,4-difluoropiperidin-1-yl)-7-methylquinoxalin-5-yl)ethyl)amino)benzoic acid;
3-((1-(3-(4,4-difluoropiperidin-1-yl)-2,7-dimethylquinoxalin-5-yl)ethyl)amino)-6-methylpicolinic acid;
2-((1-(2-cyano-3-(3,3-dimethylazetidin-1-yl)-7-methylquinoxalin-5-yl)ethyl)amino)benzoic acid;
6-chloro-3-((1-(2-cyano-3-(3,3-dimethylazetidin-1-yl)-7-methylquinoxalin-5-yl)ethyl)amino)picolinic acid;
2-((1-(2-cyano-7-methyl-3-morpholinoquinoxalin-5-yl)ethyl)amino)benzoic acid;
6-chloro-3-((1-(2-cyano-7-methyl-3-morpholinoquinoxalin-5-yl)ethyl)amino)picolinic acid;
2-((1-(2-cyano-7-methyl-3-(2-oxa-6-azaspiro[3.3]heptan-6-yl)quinoxalin-5-yl)ethyl)amino)benzoic acid;
6-chloro-3-((1-(2-cyano-7-methyl-3-(2-oxa-6-azaspiro[3.3]heptan-6-yl)quinoxalin-5-yl)ethyl)amino)picolinic acid;
2-((1-(2-cyano-3-(4,4-dimethylpiperidin-1-yl)-7-methylquinoxalin-5-yl)ethyl)amino)benzoic acid;
6-chloro-3-((1-(2-cyano-3-(4,4-dimethylpiperidin-1-yl)-7-methylquinoxalin-5-yl)ethyl)amino)picolinic acid;
2-((1-(2-cyano-3-(3-methoxy-3-methylazetidin-1-yl)-7-methylquinoxalin-5-yl)ethyl)amino)benzoic acid;
6-chloro-3-((1-(2-cyano-3-(3-methoxy-3-methylazetidin-1-yl)-7-methylquinoxalin-5-yl)ethyl)amino)picolinic acid;
2-((1-(2-cyano-7-methyl-3-(4-methyl-4-(trifluoromethyl)piperidin-1-yl)quinoxalin-5-yl)ethyl)amino)benzoic acid;
6-chloro-3-((1-(2-cyano-7-methyl-3-(4-methyl-4-(trifluoromethyl)piperidin-1-yl)quinoxalin-5-yl)ethyl)amino)picolinic acid;
2-((1-(2-cyano-3-cyclopropyl-7-methylquinoxalin-5-yl)ethyl)amino)benzoic acid;
2-((1-(2-cyano-7-methyl-3-(pyrrolidin-1-yl)quinoxalin-5-yl)ethyl)amino)benzoic acid;
6-chloro-3-((1-(2-cyano-7-methyl-3-(pyrrolidin-1-yl)quinoxalin-5-yl)ethyl)amino)picolinic acid;
6-chloro-3-((1-(2-cyano-3-(3,3-difluoropyrrolidin-1-yl)-7-methylquinoxalin-5-yl)ethyl)amino)picolinic acid;
2-((1-(2-cyano-3-(3,3-difluoropyrrolidin-1-yl)-7-methylquinoxalin-5-yl)ethyl)amino)benzoic acid;
2-((1-(2-cyano-3-(3-methoxypyrrolidin-1-yl)-7-methylquinoxalin-5-yl)ethyl)amino)benzoic acid;
6-chloro-3-((1-(2-cyano-3-(3-methoxypyrrolidin-1-yl)-7-methylquinoxalin-5-yl)ethyl)amino)picolinic acid;
6-chloro-3-((1-(2-cyano-3-(((2,2-difluoro-1-methylcyclopropyl)methyl)amino)-7-methylquinoxalin-5-yl)ethyl)amino)picolinic acid;
2-((1-(2-cyano-3-(((2,2-difluoro-1-methylcyclopropyl)methyl)amino)-7-methylquinoxalin-5-yl)ethyl)amino)benzoic acid;
2-((1-(2-cyano-3-((3,3-difluorocyclobutyl)amino)-7-methylquinoxalin-5-yl)ethyl)amino)benzoic acid;
6-chloro-3-((1-(2-cyano-3-((3,3-difluorocyclobutyl)amino)-7-methylquinoxalin-5-yl)ethyl)amino)picolinic acid;
6-chloro-3-((1-(2-cyano-3-(4,4-difluoro-3-methylpiperidin-1-yl)-7-methylquinoxalin-5-yl)ethyl)amino)picolinic acid;
2-((1-(2-cyano-3-(4,4-difluoro-3-methylpiperidin-1-yl)-7-methylquinoxalin-5-yl)ethyl)amino)benzoic acid;
6-chloro-3-((1-(2-cyano-3-(3,3-difluoro-4-methylpyrrolidin-1-yl)-7-methylquinoxalin-5-yl)ethyl)amino)picolinic acid;
2-((1-(2-cyano-3-(3,3-difluoro-4-methylpyrrolidin-1-yl)-7-methylquinoxalin-5-yl)ethyl)amino)benzoic acid;
6-chloro-3-((1-(2-cyano-7-methyl-3-(1,1,2,2-tetrafluoro-6-azaspiro[2.5]octan-6-yl)quinoxalin-5-yl)ethyl)amino)picolinic acid;
6-chloro-3-((1-(2-cyano-3-(3,4-dihydroisoquinolin-2(1H)-yl)-7-methylquinoxalin-5-yl)ethyl)amino)picolinic acid;
2-((1-(2-cyano-3-(3,4-dihydroisoquinolin-2(1H)-yl)-7-methylquinoxalin-5-yl)ethyl)amino)benzoic acid;
6-chloro-3-((1-(2-cyano-3-(6-cyano-3,4-dihydroisoquinolin-2(1H)-yl)-7-methylquinoxalin-5-yl)ethyl)amino)picolinic acid;
2-((1-(2-cyano-3-(6-cyano-3,4-dihydroisoquinolin-2(1H)-yl)-7-methylquinoxalin-5-yl)ethyl)amino)benzoic acid;
6-chloro-3-((1-(2-cyano-3-(6-fluoro-3,4-dihydroisoquinolin-2(1H)-yl)-7-methylquinoxalin-5-yl)ethyl)amino)picolinic acid;
2-((1-(2-cyano-3-(6-fluoro-3,4-dihydroisoquinolin-2(1H)-yl)-7-methylquinoxalin-5-yl)ethyl)amino)benzoic acid;
2-((1-(2-cyano-3-(3,4-dihydro-2,7-naphthyridin-2(1H)-yl)-7-methylquinoxalin-5-yl)ethyl)amino)benzoic acid;
6-chloro-3-((1-(2-cyano-3-(4-(2-cyclopropylacetyl)piperazin-1-yl)-7-methylquinoxalin-5-yl)ethyl)amino)picolinic acid;
2-((1-(2-cyano-3-(4-(2-cyclopropylacetyl)piperazin-1-yl)-7-methylquinoxalin-5-yl)ethyl)amino)benzoic acid;
6-chloro-3-((1-(2-cyano-3-(4-(2-(2,2-difluorocyclopropyl)acetyl)piperazin-1-yl)-7-methylquinoxalin-5-yl)ethyl)amino)picolinic acid;
2-((1-(2-cyano-3-(4-(2-(2,2-difluorocyclopropyl)acetyl)piperazin-1-yl)-7-methylquinoxalin-5-yl)ethyl)amino)benzoic acid;
6-chloro-3-((1-(2-cyano-3-(1-hydroxy-6-azaspiro[3.5]nonan-6-yl)-7-methylquinoxalin-5-yl)ethyl)amino)picolinic acid;
2-((1-(2-cyano-3-(1-hydroxy-6-azaspiro[3.5]nonan-6-yl)-7-methylquinoxalin-5-yl)ethyl)amino)benzoic acid;
6-chloro-3-((1-(2-cyano-7-methyl-3-(3-(1-methyl-1H-pyrazol-5-yl)-8-azabicyclo[3.2.1]octan-8-yl)quinoxalin-5-yl)ethyl)amino)picolinic acid;
2-((1-(2-cyano-7-methyl-3-(3-(1-methyl-1H-pyrazol-5-yl)-8-azabicyclo[3.2.1]octan-8-yl)quinoxalin-5-yl)ethyl)amino)benzoic acid;
2-((1-(2-cyano-7-methyl-3-(3-(1-methyl-1H-pyrazol-3-yl)-8-azabicyclo[3.2.1]octan-8-yl)quinoxalin-5-yl)ethyl)amino)benzoic acid;
2-((1-(2-cyano-7-methyl-3-(3-(1-methyl-1H-pyrazol-4-yl)-8-azabicyclo[3.2.1]octan-8-yl)quinoxalin-5-yl)ethyl)amino)benzoic acid;
6-chloro-3-((1-(2-cyano-7-methyl-3-(3-(1-methyl-1H-pyrazol-3-yl)-8-azabicyclo[3.2.1]octan-8-yl)quinoxalin-5-yl)ethyl)amino)picolinic acid;
6-chloro-3-((1-(2-cyano-7-methyl-3-(3-(1-methyl-1H-pyrazol-4-yl)-8-azabicyclo[3.2.1]octan-8-yl)quinoxalin-5-yl)ethyl)amino)picolinic acid;
6-chloro-3-((1-(2-cyano-7-methyl-3-(2-oxo-[1,4′-bipiperidin]-1′-yl)quinoxalin-5-yl)ethyl)amino)picolinic acid;
2-((1-(2-cyano-7-methyl-3-(2-oxo-[1,4′-bipiperidin]-1′-yl)quinoxalin-5-yl)ethyl)amino)benzoic acid;
6-chloro-3-((1-(2-cyano-3-(4-(ethoxymethyl)piperidin-1-yl)-7-methylquinoxalin-5-yl)ethyl)amino)picolinic acid;
2-((1-(2-cyano-3-(4-(ethoxymethyl)piperidin-1-yl)-7-methylquinoxalin-5-yl)ethyl)amino)benzoic acid;
or a pharmaceutically acceptable salt thereof.

65. A compound according to claim 1, which is:
(R)-2-((1-(2-cyano-3-(4,4-difluoropiperidin-1-yl)-7-methylquinoxalin-5-yl)ethyl)amino)benzoic acid;
(S)-2-((1-(2-cyano-3-(4,4-difluoropiperidin-1-yl)-7-methylquinoxalin-5-yl)ethyl)amino)benzoic acid;
(R)-6-chloro-3-((1-(2-cyano-3-(4,4-difluoropiperidin-1-yl)-7-methylquinoxalin-5-yl)ethyl)amino)picolinic acid;
(R)-2-((1-(2-cyano-3-(isoindolin-2-yl)-7-methylquinoxalin-5-yl)ethyl)amino)benzoic acid;
6-chloro-3-(((1R)-1-(2-cyano-3-(6,6-difluoro-3-azabicyclo[3.1.1]heptan-3-yl)-7-methylquinoxalin-5-yl)ethyl)amino)picolinic acid;
6-chloro-3-(((R)-1-(2-cyano-3-((1 S,4S)-5,5-difluoro-2-azabicyclo[2.2.1]heptan-2-yl)-7-methylquinoxalin-5-yl)ethyl)amino)picolinic acid;
6-chloro-3-(((R)-1-(2-cyano-3-((1R,4R)-5,5-difluoro-2-azabicyclo[2.2.1]heptan-2-yl)-7-methylquinoxalin-5-yl)ethyl)amino)picolinic acid;
6-chloro-3-(((R)-1-(2-cyano-3-((1R,4S)-6,6-difluoro-2-azabicyclo[2.2.1]heptan-2-yl)-7-methylquinoxalin-5-yl)ethyl)amino)picolinic acid;
6-chloro-3-(((R)-1-(2-cyano-3-((1S,4R)-6,6-difluoro-2-azabicyclo[2.2.1]heptan-2-yl)-7-methylquinoxalin-5-yl)ethyl)amino)picolinic acid;
(R)-6-chloro-3-((1-(2-cyano-3-(3,3-difluoropiperidin-1-yl)-7-methylquinoxalin-5-yl)ethyl)amino)picolinic acid;
6-chloro-3-(((R)-1-(2-cyano-3-((((S)-2,2-difluorocyclopropyl)methyl)amino)-7-methylquinoxalin-5-yl)ethyl)amino)picolinic acid;
6-chloro-3-(((R)-1-(2-cyano-3-((((R)-2,2-difluorocyclopropyl)methyl)amino)-7-methylquinoxalin-5-yl)ethyl)amino)picolinic acid;
(R)-6-chloro-3-((1-(2-cyano-7-methyl-3-(((1-(trifluoromethyl)cyclopropyl)methyl)amino)quinoxalin-5-yl)ethyl)amino)picolinic acid;
(R)-6-chloro-3-((1-(2-cyano-3-(1,1-difluoro-5-azaspiro[2.3]hexan-5-yl)-7-methylquinoxalin-5-yl)ethyl)amino)picolinic acid;
(R)-3-((1-(2-cyano-3-(4,4-difluoropiperidin-1-yl)-7-methylquinoxalin-5-yl)ethyl)amino)-6-methylpicolinic acid;
(R)-2-((1-(2-cyano-3-((cyclopropylmethyl)amino)-7-methylquinoxalin-5-yl)ethyl)amino)benzoic acid;
6-chloro-3-(((1R)-1-(2-cyano-7-methyl-3-(3-(trifluoromethyl)piperidin-1-yl)quinoxalin-5-yl)ethyl)amino)picolinic acid;
(R)-6-chloro-3-((1-(2-cyano-3-(4,4-difluoropiperidin-1-yl)-7-(trifluoromethyl)quinoxalin-5-yl)ethyl)amino)picolinic acid;
6-chloro-3-(((1R)-1-(2-cyano-3-(6,6-difluoro-3-azabicyclo[3.1.0]hexan-3-yl)-7-methylquinoxalin-5-yl)ethyl)amino)picolinic acid;
(R)-6-chloro-3-((1-(2-cyano-3-(4,4-difluoro-6-azaspiro[2.5]octan-6-yl)-7-methylquinoxalin-5-yl)ethyl)amino)picolinic acid;
(R)-6-chloro-3-((1-(2-cyano-3-(((1-fluorocyclopropyl)methyl)amino)-7-methylquinoxalin-5-yl)ethyl)amino)picolinic acid;
(R)-6-chloro-3-((1-(2-cyano-3-(5-fluoroisoindolin-2-yl)-7-methylquinoxalin-5-yl)ethyl)amino)picolinic acid;
(R)-6-chloro-3-((1-(2-cyano-3-((cyclopropylmethyl)amino)-7-methylquinoxalin-5-yl)ethyl)amino)picolinic acid;
(R)-6-chloro-3-((1-(2-cyano-3-(4,4-difluoropiperidin-1-yl)-7-fluoroquinoxalin-5-yl)ethyl)amino)picolinic acid;
(R)-2-((1-(2-cyano-3-(4,4-difluoropiperidin-1-yl)-7-fluoroquinoxalin-5-yl)ethyl)amino)benzoic acid;
(S)-2-((1-(2-cyano-3-(4,4-difluoropiperidin-1-yl)-7-fluoroquinoxalin-5-yl)ethyl)amino)benzoic acid;
(S)-2-((1-(2,7-dicyano-3-(4,4-difluoropiperidin-1-yl)quinoxalin-5-yl)ethyl)amino)benzoic acid;
(R)-2-((1-(2,7-dicyano-3-(4,4-difluoropiperidin-1-yl)quinoxalin-5-yl)ethyl)amino)benzoic acid;
(S)-2-((1-(7-chloro-2-cyano-3-(4,4-difluoropiperidin-1-yl)quinoxalin-5-yl)ethyl)amino)benzoic acid;
(R)-2-((1-(7-chloro-2-cyano-3-(4,4-difluoropiperidin-1-yl)quinoxalin-5-yl)ethyl)amino)benzoic acid;
(R)-6-chloro-3-((1-(2-cyano-7-cyclopropyl-3-(4,4-difluoropiperidin-1-yl)quinoxalin-5-yl)ethyl)amino)picolinic acid;
(R)-6-chloro-3-((1-(7-chloro-2-cyano-3-(4,4-difluoropiperidin-1-yl)quinoxalin-5-yl)ethyl)amino)picolinic acid;
(R)-6-chloro-3-((1-(3-(4,4-difluoropiperidin-1-yl)-2,7-dimethylquinoxalin-5-yl)ethyl)amino)picolinic acid;
(R)-2-((1-(3-(4,4-difluoropiperidin-1-yl)-2,7-dimethylquinoxalin-5-yl)ethyl)amino)benzoic acid;
(R)-2-((1-(2-cyano-7-cyclopropyl-3-(4,4-difluoropiperidin-1-yl)quinoxalin-5-yl)ethyl)amino)benzoic acid;
(R)-2-((1-(2-cyano-3-(4,4-difluoropiperidin-1-yl)-7-methoxyquinoxalin-5-yl)ethyl)amino)benzoic acid;
(R)-5-((1-(2-cyano-3-(4,4-difluoropiperidin-1-yl)-7-methylquinoxalin-5-yl)ethyl)amino)thiazole-4-carboxylic acid;
(R)-2-chloro-5-((1-(2-cyano-3-(4,4-difluoropiperidin-1-yl)-7-methylquinoxalin-5-yl)ethyl)amino)thiazole-4-carboxylic acid;
(R)-2-((1-(2-cyano-3-(4,4-difluoropiperidin-1-yl)-7-methylquinoxalin-5-yl)ethyl)amino)thiophene-3-carboxylic acid;
(R)-3-((1-(2-cyano-3-(4,4-difluoropiperidin-1-yl)-7-methylquinoxalin-5-yl)ethyl)amino)thiophene-2-carboxylic acid;
6-chloro-3-(((1R)-1-(2-cyano-7-methyl-3-((1 S)-1-(trifluoromethyl)-3-azabicyclo[3.1.0]hexan-3-yl)quinoxalin-5-yl)ethyl)amino)picolinic acid;
(R)-6-chloro-3-((1-(2-cyano-3-cyclopropyl-7-methylquinoxalin-5-yl)ethyl)amino)picolinic acid;
6-chloro-3-(((R)-1-(2-cyano-7-methyl-3-((R)-3-(trifluoromethyl)piperidin-1-yl)quinoxalin-5-yl)ethyl)amino)picolinic acid;
6-chloro-3-(((R)-1-(2-cyano-7-methyl-3-((S)-3-(trifluoromethyl)piperidin-1-yl)quinoxalin-5-yl)ethyl)amino)picolinic acid;
6-chloro-3-(((1R)-1-(2-cyano-7-methyl-3-((1R)-1-(trifluoromethyl)-3-azabicyclo[3.1.0]hexan-3-yl)quinoxalin-5-yl)ethyl)amino)picolinic acid;
(R)-6-chloro-3-((1-(2-cyclopropyl-3-(4,4-difluoropiperidin-1-yl)-7-methylquinoxalin-5-yl)ethyl)amino)picolinic acid;
(R)-6-chloro-3-((1-(3-(4,4-difluoropiperidin-1-yl)-7-methyl-2-(trifluoromethyl)quinoxalin-5-yl)ethyl)amino)picolinic acid;
(R)-2-((1-(3-(4,4-difluoropiperidin-1-yl)-7-methyl-2-(trifluoromethyl)quinoxalin-5-yl)ethyl)amino)benzoic acid;
(R)-2-((1-(2-cyano-3-(4,4-difluoropiperidin-1-yl)-7-ethylquinoxalin-5-yl)ethyl)amino)benzoic acid;
(R)-6-chloro-3-((1-(2-chloro-3-(4,4-difluoropiperidin-1-yl)-7-methylquinoxalin-5-yl)ethyl)amino)picolinic acid;
(R)-2-((1-(2-chloro-3-(4,4-difluoropiperidin-1-yl)-7-methylquinoxalin-5-yl)ethyl)amino)benzoic acid;
(R)-3-((1-(3-(4,4-difluoropiperidin-1-yl)-2,7-dimethylquinoxalin-5-yl)ethyl)amino)-6-methylpicolinic acid;
(R)-2-((1-(2-cyano-3-(3,3-dimethylazetidin-1-yl)-7-methylquinoxalin-5-yl)ethyl)amino)benzoic acid;
(R)-6-chloro-3-((1-(2-cyano-3-(3,3-dimethylazetidin-1-yl)-7-methylquinoxalin-5-yl)ethyl)amino)picolinic acid;
(R)-2-((1-(2-cyano-7-methyl-3-morpholinoquinoxalin-5-yl)ethyl)amino)benzoic acid;
(R)-6-chloro-3-((1-(2-cyano-7-methyl-3-morpholinoquinoxalin-5-yl)ethyl)amino)picolinic acid;
(R)-2-((1-(2-cyano-7-methyl-3-(2-oxa-6-azaspiro[3.3]heptan-6-yl)quinoxalin-5-yl)ethyl)amino)benzoic acid;
(R)-6-chloro-3-((1-(2-cyano-7-methyl-3-(2-oxa-6-azaspiro[3.3]heptan-6-yl)quinoxalin-5-yl)ethyl)amino)picolinic acid;
(R)-2-((1-(2-cyano-3-(4,4-dimethylpiperidin-1-yl)-7-methylquinoxalin-5-yl)ethyl)amino)benzoic acid;
(R)-6-chloro-3-((1-(2-cyano-3-(4,4-dimethylpiperidin-1-yl)-7-methylquinoxalin-5-yl)ethyl)amino)picolinic acid;
(R)-2-((1-(2-cyano-3-(3-methoxy-3-methylazetidin-1-yl)-7-methylquinoxalin-5-yl)ethyl)amino)benzoic acid;
(R)-6-chloro-3-((1-(2-cyano-3-(3-methoxy-3-methylazetidin-1-yl)-7-methylquinoxalin-5-yl)ethyl)amino)picolinic acid;
(R)-2-((1-(2-cyano-7-methyl-3-(4-methyl-4-(trifluoromethyl)piperidin-1-yl)quinoxalin-5-yl)ethyl)amino)benzoic acid;
(R)-6-chloro-3-((1-(2-cyano-7-methyl-3-(4-methyl-4-(trifluoromethyl)piperidin-1-yl)quinoxalin-5-yl)ethyl)amino)picolinic acid;
(R)-2-((1-(2-cyano-3-cyclopropyl-7-methylquinoxalin-5-yl)ethyl)amino)benzoic acid;
(R)-2-((1-(2-cyano-7-methyl-3-(pyrrolidin-1-yl)quinoxalin-5-yl)ethyl)amino)benzoic acid;
(R)-6-chloro-3-((1-(2-cyano-7-methyl-3-(pyrrolidin-1-yl)quinoxalin-5-yl)ethyl)amino)picolinic acid;
(R)-6-chloro-3-((1-(2-cyano-3-(3,3-difluoropyrrolidin-1-yl)-7-methylquinoxalin-5-yl)ethyl)amino)picolinic acid;
(R)-2-((1-(2-cyano-3-(3,3-difluoropyrrolidin-1-yl)-7-methylquinoxalin-5-yl)ethyl)amino)benzoic acid;
2-(((1R)-1-(2-cyano-3-(3-methoxypyrrolidin-1-yl)-7-methylquinoxalin-5-yl)ethyl)amino)benzoic acid;
6-chloro-3-(((1R)-1-(2-cyano-3-(3-methoxypyrrolidin-1-yl)-7-methylquinoxalin-5-yl)ethyl)amino)picolinic acid;
6-chloro-3-(((1R)-1-(2-cyano-3-(((2,2-difluoro-1-methylcyclopropyl)methyl)amino)-7-methylquinoxalin-5-yl)ethyl)amino)picolinic acid;
2-(((1R)-1-(2-cyano-3-(((2,2-difluoro-1-methylcyclopropyl)methyl)amino)-7-methylquinoxalin-5-yl)ethyl)amino)benzoic acid;
2-(((R)-1-(2-cyano-3-((((S)-2,2-difluoro-1-methylcyclopropyl)methyl)amino)-7-methylquinoxalin-5-yl)ethyl)amino)benzoic acid;
2-(((R)-1-(2-cyano-3-((((R)-2,2-difluoro-1-methylcyclopropyl)methyl)amino)-7-methylquinoxalin-5-yl)ethyl)amino)benzoic acid;
(R)-2-((1-(2-cyano-3-((3,3-difluorocyclobutyl)amino)-7-methylquinoxalin-5-yl)ethyl)amino)benzoic acid;
(R)-6-chloro-3-((1-(2-cyano-3-((3,3-difluorocyclobutyl)amino)-7-methylquinoxalin-5-yl)ethyl)amino)picolinic acid;
6-chloro-3-(((1R)-1-(2-cyano-3-(4,4-difluoro-3-methylpiperidin-1-yl)-7-methylquinoxalin-5-yl)ethyl)amino)picolinic acid;
2-(((1R)-1-(2-cyano-3-(4,4-difluoro-3-methylpiperidin-1-yl)-7-methylquinoxalin-5-yl)ethyl)amino)benzoic acid;
6-chloro-3-(((1R)-1-(2-cyano-3-(3,3-difluoro-4-methylpyrrolidin-1-yl)-7-methylquinoxalin-5-yl)ethyl)amino)picolinic acid;
2-(((1R)-1-(2-cyano-3-(3,3-difluoro-4-methylpyrrolidin-1-yl)-7-methylquinoxalin-5-yl)ethyl)amino)benzoic acid;
(R)-6-chloro-3-((1-(2-cyano-7-methyl-3-(1,1,2,2-tetrafluoro-6-azaspiro[2.5]octan-6-yl)quinoxalin-5-yl)ethyl)amino)picolinic acid;
(R)-6-chloro-3-((1-(2-cyano-3-(3,4-dihydroisoquinolin-2(1H)-yl)-7-methylquinoxalin-5-yl)ethyl)amino)picolinic acid;
(R)-2-((1-(2-cyano-3-(3,4-dihydroisoquinolin-2(1H)-yl)-7-methylquinoxalin-5-yl)ethyl)amino)benzoic acid;
(R)-6-chloro-3-((1-(2-cyano-3-(6-cyano-3,4-dihydroisoquinolin-2(1H)-yl)-7-methylquinoxalin-5-yl)ethyl)amino)picolinic acid;
(R)-2-((1-(2-cyano-3-(6-cyano-3,4-dihydroisoquinolin-2(1H)-yl)-7-methylquinoxalin-5-yl)ethyl)amino)benzoic acid;
(R)-6-chloro-3-((1-(2-cyano-3-(6-fluoro-3,4-dihydroisoquinolin-2(1H)-yl)-7-methylquinoxalin-5-yl)ethyl)amino)picolinic acid;
(R)-2-((1-(2-cyano-3-(6-fluoro-3,4-dihydroisoquinolin-2(1H)-yl)-7-methylquinoxalin-5-yl)ethyl)amino)benzoic acid;
(R)-2-((1-(2-cyano-3-(3,4-dihydro-2,7-naphthyridin-2(1H)-yl)-7-methylquinoxalin-5-yl)ethyl)amino)benzoic acid;
(R)-6-chloro-3-((1-(2-cyano-3-(4-(2-cyclopropylacetyl)piperazin-1-yl)-7-methylquinoxalin-5-yl)ethyl)amino)picolinic acid;
(R)-2-((1-(2-cyano-3-(4-(2-cyclopropylacetyl)piperazin-1-yl)-7-methylquinoxalin-5-yl)ethyl)amino)benzoic acid;
6-chloro-3-(((1R)-1-(2-cyano-3-(4-(2-(2,2-difluorocyclopropyl)acetyl)piperazin-1-yl)-7-methylquinoxalin-5-yl)ethyl)amino)picolinic acid;
6-chloro-3-(((R)-1-(2-cyano-3-(4-(2-((S)-2,2-difluorocyclopropyl)acetyl)piperazin-1-yl)-7-methylquinoxalin-5-yl)ethyl)amino)picolinic acid;
6-chloro-3-(((R)-1-(2-cyano-3-(4-(2-((R)-2,2-difluorocyclopropyl)acetyl)piperazin-1-yl)-7-methylquinoxalin-5-yl)ethyl)amino)picolinic acid;
2-(((1R)-1-(2-cyano-3-(4-(2-(2,2-difluorocyclopropyl)acetyl)piperazin-1-yl)-7-methylquinoxalin-5-yl)ethyl)amino)benzoic acid;
6-chloro-3-(((1R)-1-(2-cyano-3-(1-hydroxy-6-azaspiro[3.5]nonan-6-yl)-7-methylquinoxalin-5-yl)ethyl)amino)picolinic acid;
2-(((1R)-1-(2-cyano-3-(1-hydroxy-6-azaspiro[3.5]nonan-6-yl)-7-methylquinoxalin-5-yl)ethyl)amino)benzoic acid;
2-(((1R)-1-(2-cyano-3-((1R)-1-hydroxy-6-azaspiro[3.5]nonan-6-yl)-7-methylquinoxalin-5-yl)ethyl)amino)benzoic acid;
2-(((1R)-1-(2-cyano-3-((1 S)-1-hydroxy-6-azaspiro[3.5]nonan-6-yl)-7-methylquinoxalin-5-yl)ethyl)amino)benzoic acid;
6-chloro-3-(((1R)-1-(2-cyano-7-methyl-3-(3-(1-methyl-1H-pyrazol-5-yl)-8-azabicyclo[3.2.1]octan-8-yl)quinoxalin-5-yl)ethyl)amino)picolinic acid;
6-chloro-3-(((1R)-1-(2-cyano-7-methyl-3-(3-(1-methyl-1H-pyrazol-5-yl)-8-azabicyclo[3.2.1]octan-8-yl)quinoxalin-5-yl)ethyl)amino)picolinic acid;
6-chloro-3-(((1R)-1-(2-cyano-7-methyl-3-(3-(1-methyl-1H-pyrazol-5-yl)-8-azabicyclo[3.2.1]octan-8-yl)quinoxalin-5-yl)ethyl)amino)picolinic acid;
2-(((1R)-1-(2-cyano-7-methyl-3-(3-(1-methyl-1H-pyrazol-5-yl)-8-azabicyclo[3.2.1]octan-8-yl)quinoxalin-5-yl)ethyl)amino)benzoic acid;
2-(((1R)-1-(2-cyano-7-methyl-3-((1R,5S)-3-(1-methyl-1H-pyrazol-5-yl)-8-azabicyclo[3.2.1]octan-8-yl)quinoxalin-5-yl)ethyl)amino)benzoic acid;
2-(((1R)-1-(2-cyano-7-methyl-3-((1R,5S)-3-(1-methyl-1H-pyrazol-5-yl)-8-azabicyclo[3.2.1]octan-8-yl)quinoxalin-5-yl)ethyl)amino)benzoic acid;
2-(((1R)-1-(2-cyano-7-methyl-3-(3-(1-methyl-1H-pyrazol-3-yl)-8-azabicyclo[3.2.1]octan-8-yl)quinoxalin-5-yl)ethyl)amino)benzoic acid;
2-(((1R)-1-(2-cyano-7-methyl-3-((1R,5S)-3-(1-methyl-1H-pyrazol-3-yl)-8-azabicyclo[3.2.1]octan-8-yl)quinoxalin-5-yl)ethyl)amino)benzoic acid;
2-(((1R)-1-(2-cyano-7-methyl-3-((1R,5S)-3-(1-methyl-1H-pyrazol-3-yl)-8-azabicyclo[3.2.1]octan-8-yl)quinoxalin-5-yl)ethyl)amino)benzoic acid;
2-(((1R)-1-(2-cyano-7-methyl-3-(3-(1-methyl-1H-pyrazol-4-yl)-8-azabicyclo[3.2.1]octan-8-yl)quinoxalin-5-yl)ethyl)amino)benzoic acid;
2-(((1R)-1-(2-cyano-7-methyl-3-((1R,5S)-3-(1-methyl-1H-pyrazol-4-yl)-8-azabicyclo[3.2.1]octan-8-yl)quinoxalin-5-yl)ethyl)amino)benzoic acid;
2-(((1R)-1-(2-cyano-7-methyl-3-((1R,5S)-3-(1-methyl-1H-pyrazol-4-yl)-8-azabicyclo[3.2.1]octan-8-yl)quinoxalin-5-yl)ethyl)amino)benzoic acid;
6-chloro-3-(((1R)-1-(2-cyano-7-methyl-3-(3-(1-methyl-1H-pyrazol-3-yl)-8-azabicyclo[3.2.1]octan-8-yl)quinoxalin-5-yl)ethyl)amino)picolinic acid;
6-chloro-3-(((1R)-1-(2-cyano-7-methyl-3-(3-(1-methyl-1H-pyrazol-4-yl)-8-azabicyclo[3.2.1]octan-8-yl)quinoxalin-5-yl)ethyl)amino)picolinic acid;
6-chloro-3-(((1R)-1-(2-cyano-7-methyl-3-(3-(2-oxopiperidin-1-yl)-8-azabicyclo[3.2.1]octan-8-yl)quinoxalin-5-yl)ethyl)amino)picolinic acid;
(R)-2-((1-(2-cyano-7-methyl-3-(2-oxo-[1,4′-bipiperidin]-1′-yl)quinoxalin-5-yl)ethyl)amino)benzoic acid;
(R)-6-chloro-3-((1-(2-cyano-3-(4-(ethoxymethyl)piperidin-1-yl)-7-methylquinoxalin-5-yl)ethyl)amino)picolinic acid;
(R)-2-((1-(2-cyano-3-(4-(ethoxymethyl)piperidin-1-yl)-7-methylquinoxalin-5-yl)ethyl)amino)benzoic acid;
or a pharmaceutically acceptable salt thereof.

66. A compound according to claim 1, which is:
(R)-6-chloro-3-((1-(2-cyano-7-methyl-3-(4-(methylsulfonyl)piperidin-1-yl)quinoxalin-5-yl)ethyl)amino)picolinic acid;
(R)-6-chloro-3-((1-(2-cyano-7-methyl-3-(1-oxidothiomorpholino)quinoxalin-5-yl)ethyl)amino)picolinic acid;
(R)-6-chloro-3-((1-(2-cyano-3-((2-methoxyethyl)(methyl)amino)-7-methylquinoxalin-5-yl)ethyl)amino)picolinic acid;
2-(((1R)-1-(2-cyano-3-(4,4-difluoro-2-methylpyrrolidin-1-yl)-7-methylquinoxalin-5-yl)ethyl)amino)benzoic acid;
6-chloro-3-(((R)-1-(2-cyano-3-((R)-4,4-difluoro-2-methylpyrrolidin-1-yl)-7-methylquinoxalin-5-yl)ethyl)amino)picolinic acid;
6-chloro-3-(((R)-1-(2-cyano-3-((S)-4,4-difluoro-2-methylpyrrolidin-1-yl)-7-methylquinoxalin-5-yl)ethyl)amino)picolinic acid;
6-chloro-3-(((R)-1-(2-cyano-3-((S)-3-(hydroxymethyl)piperidin-1-yl)-7-methylquinoxalin-5-yl)ethyl)amino)picolinic acid;
6-chloro-3-(((R)-1-(2-cyano-3-((R)-3-(hydroxymethyl)piperidin-1-yl)-7-methylquinoxalin-5-yl)ethyl)amino)picolinic acid;
(R)-3-((1-(2-cyano-3-(4,4-difluoropiperidin-1-yl)-7-methylquinoxalin-5-yl)ethyl)amino)pyrazine-2-carboxylic acid;
(R)-6-chloro-3-((1-(2-cyano-3-((3-(dimethylamino)-3-oxopropyl)(methyl)amino)-7-methylquinoxalin-5-yl)ethyl)amino)picolinic acid;
(R)-6-chloro-3-((1-(2-cyano-7-methyl-3-(4-(5-methylthiazol-2-yl)piperazin-1-yl)quinoxalin-5-yl)ethyl)amino)picolinic acid;
(R)-6-chloro-3-((1-(2-cyano-3-(7,7-difluoro-5-azaspiro[2.4]heptan-5-yl)-7-methylquinoxalin-5-yl)ethyl)amino)picolinic acid;
(R)-6-chloro-3-((1-(2-cyano-3-(1H-imidazol-1-yl)-7-methylquinoxalin-5-yl)ethyl)amino)picolinic acid;
(R)-3-((1-(2-cyano-3-(4,4-difluoropiperidin-1-yl)-7-methylquinoxalin-5-yl)ethyl)amino)picolinic acid;
(R)-6-chloro-3-((1-(2-cyano-3-(4-(ethoxymethyl)piperidin-1-yl)-7-methylquinoxalin-5-yl)ethyl)amino)picolinic acid;
3-(((1R)-1-(3-(3-oxa-8-azabicyclo[3.2.1]octan-8-yl)-2-cyano-7-methylquinoxalin-5-yl)ethyl)amino)-6-chloropicolinic acid;
(R)-4-((1-(2-cyano-3-(4,4-difluoropiperidin-1-yl)-7-methylquinoxalin-5-yl)ethyl)amino)thiophene-3-carboxylic acid;
6-chloro-3-(((R)-1-(2-cyano-3-((3S,4R)-3,4-difluoropyrrolidin-1-yl)-7-methylquinoxalin-5-yl)ethyl)amino)picolinic acid;
(R)-6-chloro-3-((1-(2-cyano-3-(3,3-difluoro-4,4-dimethylpyrrolidin-1-yl)-7-methylquinoxalin-5-yl)ethyl)amino)picolinic acid;
(R)-6-chloro-3-((1-(2-cyano-7-methyl-3-((3,3,3-trifluoropropyl)amino)quinoxalin-5-yl)ethyl)amino)picolinic acid;
(R)-6-chloro-3-((1-(2-cyano-3-((3-(dimethylamino)-3-oxopropyl)amino)-7-methylquinoxalin-5-yl)ethyl)amino)picolinic acid;
(R)-6-chloro-3-((1-(2-cyano-7-methyl-3-(4-(1-methyl-1H-pyrazol-5-yl)piperidin-1-yl)quinoxalin-5-yl)ethyl)amino)picolinic acid;
(R)-2-((1-(2-cyano-3-(3,3-difluoroazetidin-1-yl)-7-methylquinoxalin-5-yl)ethyl)amino)benzoic acid;
(R)-6-chloro-3-((1-(2-cyano-3-(3,3-difluoroazetidin-1-yl)-7-methylquinoxalin-5-yl)ethyl)amino)picolinic acid;
(R)-2-((1-(2-cyano-7-methyl-3-(1,1,2,2-tetrafluoro-6-azaspiro[2.5]octan-6-yl)quinoxalin-5-yl)ethyl)amino)benzoic acid;
(R)-6-chloro-3-((1-(2-cyano-7-methyl-3-(1-oxa-6-azaspiro[3.3]heptan-6-yl)quinoxalin-5-yl)ethyl)amino)picolinic acid;
(R)-6-chloro-3-((1-(2-cyano-7-methyl-3-(3-(trifluoromethyl)azetidin-1-yl)quinoxalin-5-yl)ethyl)amino)picolinic acid;
(R)-6-chloro-3-((1-(2-cyano-3-((2-methoxyethyl)amino)-7-methylquinoxalin-5-yl)ethyl)amino)picolinic acid;
(R)-6-chloro-3-((1-(2-cyano-7-methyl-3-(4-(6-(trifluoromethyl)pyridin-3-yl)piperazin-1-yl)quinoxalin-5-yl)ethyl)amino)picolinic acid;
(R)-2-((1-(2-cyano-7-methyl-3-(1-oxa-6-azaspiro[3.3]heptan-6-yl)quinoxalin-5-yl)ethyl)amino)benzoic acid;
(R)-6-chloro-3-((1-(2-cyano-7-methyl-3-((oxetan-3-ylmethyl)amino)quinoxalin-5-yl)ethyl)amino)picolinic acid;
(R)-3-((1-(3-(4-(1H-pyrazol-1-yl)piperidin-1-yl)-2-cyano-7-methylquinoxalin-5-yl)ethyl)amino)-6-chloropicolinic acid;
(R)-6-chloro-3-((1-(2-cyano-7-methyl-3-(4-(N-methylacetamido)piperidin-1-yl)quinoxalin-5-yl)ethyl)amino)picolinic acid;
(R)-6-chloro-3-((1-(2-cyano-7-methyl-3-(4-(4-methyl-1H-pyrazol-1-yl)piperidin-1-yl)quinoxalin-5-yl)ethyl)amino)picolinic acid;
(R)-6-chloro-3-((1-(2-cyano-7-methyl-3-(4-(4-(trifluoromethyl)-1H-pyrazol-1-yl)piperidin-1-yl)quinoxalin-5-yl)ethyl)amino)picolinic acid;
6-chloro-3-(((R)-1-(2-cyano-7-methyl-3-cis-tetrahydro-1H-furo[3,4-c]pyrrol-5(3H)-yl)quinoxalin-5-yl)ethyl)amino)picolinic acid;
(R)-6-chloro-3-((1-(2-cyano-7-methyl-3-(4-oxa-7-azaspiro[2.5]octan-7-yl)quinoxalin-5-yl)ethyl)amino)picolinic acid;
6-chloro-3-(((R)-1-(2-cyano-7-methyl-3-((R)-3-(methylsulfonyl)pyrrolidin-1-yl)quinoxalin-5-yl)ethyl)amino)picolinic acid;
6-chloro-3-(((R)-1-(2-cyano-3-((3S,5R)-3,5-difluoropiperidin-1-yl)-7-methylquinoxalin-5-yl)ethyl)amino)picolinic acid;
(R)-6-chloro-3-((1-(2-cyano-3-(2,2-dimethylmorpholino)-7-methylquinoxalin-5-yl)ethyl)amino)picolinic acid;
(R)-6-chloro-3-((1-(2-cyano-7-methyl-3-(2-oxa-6-azaspiro[3.3]heptan-6-yl)quinoxalin-5-yl)ethyl)amino)picolinic acid;
(R)-6-chloro-3-((1-(7-chloro-2-cyano-3-(3,3-difluoropyrrolidin-1-yl)quinoxalin-5-yl)ethyl)amino)picolinic acid;
6-chloro-3-(((R)-1-(2-cyano-3-((R)-3,3-difluoro-5-(hydroxymethyl)piperidin-1-yl)-7-methylquinoxalin-5-yl)ethyl)amino)picolinic acid;
6-chloro-3-(((R)-1-(2-cyano-3-((S)-3,3-difluoro-5-(hydroxymethyl)piperidin-1-yl)-7-methylquinoxalin-5-yl)ethyl)amino)picolinic acid;
6-chloro-3-(((R)-1-(2-cyano-3-((R)-3-(dimethylcarbamoyl)pyrrolidin-1-yl)-7-methylquinoxalin-5-yl)ethyl)amino)picolinic acid;
6-chloro-3-(((R)-1-(2-cyano-3-((S)-3-(dimethylcarbamoyl)pyrrolidin-1-yl)-7-methylquinoxalin-5-yl)ethyl)amino)picolinic acid;
6-chloro-3-(((R)-1-(2-cyano-7-methyl-3-((1R,5S,6R)-6-(trifluoromethyl)-3-azabicyclo[3.1.0]hexan-3-yl)quinoxalin-5-yl)ethyl)amino)picolinic acid;
(R)-6-chloro-3-((1-(2-cyano-3-(4,4-difluoropiperidin-1-yl)-7-ethylquinoxalin-5-yl)ethyl)amino)picolinic acid;
6-chloro-3-(((R)-1-(2-cyano-7-methyl-3-((1R,5S,6S)-6-(trifluoromethyl)-3-azabicyclo[3.1.0]hexan-3-yl)quinoxalin-5-yl)ethyl)amino)picolinic acid;
(R)-6-chloro-3-((1-(2-cyano-7-methyl-3-(methyl(3,3,3-trifluoropropyl)amino)quinoxalin-5-yl)ethyl)amino)picolinic acid;
6-chloro-3-(((R)-1-(2-cyano-3-((S)-3-cyanopyrrolidin-1-yl)-7-methylquinoxalin-5-yl)ethyl)amino)picolinic acid;
6-chloro-3-(((R)-1-(2-cyano-3-((R)-3-cyanopyrrolidin-1-yl)-7-methylquinoxalin-5-yl)ethyl)amino)picolinic acid;
(R)-6-chloro-3-((1-(7-chloro-2-cyano-3-morpholinoquinoxalin-5-yl)ethyl)amino)picolinic acid;
6-chloro-3-(((R)-1-(2-cyano-3-((S)-3-(hydroxymethyl)pyrrolidin-1-yl)-7-methylquinoxalin-5-yl)ethyl)amino)picolinic acid;
6-chloro-3-(((R)-1-(2-cyano-3-((R)-3-(hydroxymethyl)pyrrolidin-1-yl)-7-methylquinoxalin-5-yl)ethyl)amino)picolinic acid;
(R)-3-((1-(3-(4-acetylpiperazin-1-yl)-2-cyano-7-methylquinoxalin-5-yl)ethyl)amino)-6-chloropicolinic acid;
(R)-6-chloro-3-((1-(2-cyano-7-methyl-3-(4-oxopiperidin-1-yl)quinoxalin-5-yl)ethyl)amino)picolinic acid;
(R)-6-chloro-3-((1-(2-cyano-3-(3,4-dihydro-2,7-naphthyridin-2(1H)-yl)-7-methylquinoxalin-5-yl)ethyl)amino)picolinic acid;
6-chloro-3-(((R)-1-(2-cyano-3-((1R,5S,6S)-6-(hydroxymethyl)-3-azabicyclo[3.1.0]hexan-3-yl)-7-methylquinoxalin-5-yl)ethyl)amino)picolinic acid;
6-chloro-3-(((R)-1-(2-cyano-3-((R)-3-fluoro-3-(methoxymethyl)pyrrolidin-1-yl)-7-methylquinoxalin-5-yl)ethyl)amino)picolinic acid;
6-chloro-3-(((R)-1-(2-cyano-3-((S)-3-fluoro-3-(methoxymethyl)pyrrolidin-1-yl)-7-methylquinoxalin-5-yl)ethyl)amino)picolinic acid;
6-chloro-3-(((R)-1-(2-cyano-3-((1R,5S,6R)-6-(methoxymethyl)-3-azabicyclo[3.1.0]hexan-3-yl)-7-methylquinoxalin-5-yl)ethyl)amino)picolinic acid;
6-chloro-3-(((R)-1-(2-cyano-3-((1R,5S,6R)-6-(hydroxymethyl)-3-azabicyclo[3.1.0]hexan-3-yl)-7-methylquinoxalin-5-yl)ethyl)amino)picolinic acid;
6-chloro-3-(((R)-1-(2-cyano-7-methyl-3-((R)-2-methylmorpholino)quinoxalin-5-yl)ethyl)amino)picolinic acid;
6-chloro-3-(((R)-1-(2-cyano-3-((S)-3,3-difluoro-2-methylpyrrolidin-1-yl)-7-methylquinoxalin-5-yl)ethyl)amino)picolinic acid;
6-chloro-3-(((R)-1-(2-cyano-3-((R)-3,3-difluoro-2-methylpyrrolidin-1-yl)-7-methylquinoxalin-5-yl)ethyl)amino)picolinic acid;
6-chloro-3-(((R)-1-(2-cyano-3-((S)-3,3-difluoro-4-methoxypyrrolidin-1-yl)-7-methylquinoxalin-5-yl)ethyl)amino)picolinic acid;
6-chloro-3-(((R)-1-(2-cyano-3-((R)-3,3-difluoro-4-methoxypyrrolidin-1-yl)-7-methylquinoxalin-5-yl)ethyl)amino)picolinic acid;
3-(((R)-1-(3-((1S,4S)-2-oxa-5-azabicyclo[2.2.1]heptan-5-yl)-2-cyano-7-methylquinoxalin-5-yl)ethyl)amino)-6-chloropicolinic acid;
3-(((R)-1-(3-((1R,4R)-2-oxa-5-azabicyclo[2.2.1]heptan-5-yl)-2-cyano-7-methylquinoxalin-5-yl)ethyl)amino)-6-chloropicolinic acid;
6-chloro-3-(((R)-1-(2-cyano-7-methyl-3-((S)-3-(methylsulfonyl)pyrrolidin-1-yl)quinoxalin-5-yl)ethyl)amino)picolinic acid;
6-chloro-3-(((R)-1-(2-cyano-3-((S)-3-fluoropyrrolidin-1-yl)-7-methylquinoxalin-5-yl)ethyl)amino)picolinic acid;
6-chloro-3-(((R)-1-(2-cyano-3-((R)-3-fluoropyrrolidin-1-yl)-7-methylquinoxalin-5-yl)ethyl)amino)picolinic acid;
6-chloro-3-(((R)-1-(2-cyano-3-((3S,4S)-3,4-difluoropyrrolidin-1-yl)-7-methylquinoxalin-5-yl)ethyl)amino)picolinic acid;
(R)-6-chloro-3-((1-(2-cyano-7-methyl-3-(4-(5-(trifluoromethyl)pyridin-2-yl)piperazin-1-yl)quinoxalin-5-yl)ethyl)amino)picolinic acid;
methyl 6-chloro-3-(((R)-1-(2-cyano-3-((R)-3-fluoro-3-methylpyrrolidin-1-yl)-7-methylquinoxalin-5-yl)ethyl)amino)picolinate
methyl 6-chloro-3-(((R)-1-(2-cyano-3-((S)-3-fluoro-3-methylpyrrolidin-1-yl)-7-methylquinoxalin-5-yl)ethyl)amino)picolinate
6-chloro-3-(((R)-1-(2-cyano-3-((((S)-2,2-difluoro-1-methylcyclopropyl)methyl)amino)-7-methylquinoxalin-5-yl)ethyl)amino)picolinic acid;
6-chloro-3-(((R)-1-(2-cyano-3-((((R)-2,2-difluoro-1-methylcyclopropyl)methyl)amino)-7-methylquinoxalin-5-yl)ethyl)amino)picolinic acid;
(R)-6-chloro-3-((1-(2-cyano-7-methyl-3-(3-(N-methylacetamido)azetidin-1-yl)quinoxalin-5-yl)ethyl)amino)picolinic acid;
6-chloro-3-(((1R)-1-(2-cyano-7-methyl-3-(tetrahydrofuran-3-yl)quinoxalin-5-yl)ethyl)amino)picolinic acid;
6-chloro-3-(((R)-1-(2-cyano-7-methyl-3-((S)-tetrahydrofuran-3-yl)quinoxalin-5-yl)ethyl)amino)picolinic acid;
6-chloro-3-(((R)-1-(2-cyano-7-methyl-3-((R)-tetrahydrofuran-3-yl)quinoxalin-5-yl)ethyl)amino)picolinic acid;
(R)-6-chloro-3-((1-(2-cyano-3-(3,3-difluoropyrrolidin-1-yl)-7-ethylquinoxalin-5-yl)ethyl)amino)picolinic acid;
(R)-6-chloro-3-((1-(2-cyano-7-ethyl-3-morpholinoquinoxalin-5-yl)ethyl)amino)picolinic acid;
(R)-6-chloro-3-((1-(2-cyano-7-ethyl-3-(2-oxa-6-azaspiro[3.3]heptan-6-yl)quinoxalin-5-yl)ethyl)amino)picolinic acid;
6-chloro-3-(((R)-1-(2-cyano-3-((S)-2-ethylmorpholino)-7-methylquinoxalin-5-yl)ethyl)amino)picolinic acid;
6-chloro-3-(((R)-1-(2-cyano-3-((R)-2-ethylmorpholino)-7-methylquinoxalin-5-yl)ethyl)amino)picolinic acid;
6-chloro-3-(((R)-1-(2-cyano-7-methyl-3-((S)-2-(trifluoromethyl)morpholino)quinoxalin-5-yl)ethyl)amino)picolinic acid;
6-chloro-3-(((R)-1-(2-cyano-7-methyl-3-((R)-2-(trifluoromethyl)morpholino)quinoxalin-5-yl)ethyl)amino)picolinic acid;
6-chloro-3-(((R)-1-(2-cyano-3-((S)-2-(difluoromethyl)morpholino)-7-methylquinoxalin-5-yl)ethyl)amino)picolinic acid;
6-chloro-3-(((R)-1-(2-cyano-3-((R)-2-(difluoromethyl)morpholino)-7-methylquinoxalin-5-yl)ethyl)amino)picolinic acid;
6-chloro-3-(((R)-1-(2-cyano-7-methyl-3-((S)-3-methylmorpholino)quinoxalin-5-yl)ethyl)amino)picolinic acid;
6-chloro-3-(((R)-1-(2-cyano-7-methyl-3-((R)-3-methylmorpholino)quinoxalin-5-yl)ethyl)amino)picolinic acid;
3-(((1R)-1-(3-(6-oxa-3-azabicyclo[3.1.1]heptan-3-yl)-2-cyano-7-methylquinoxalin-5-yl)ethyl)amino)-6-chloropicolinic acid;
6-chloro-3-(((R)-1-(2-cyano-3-(4-((R)-1-hydroxyethyl)piperidin-1-yl)-7-methylquinoxalin-5-yl)ethyl)amino)picolinic acid;
6-chloro-3-(((R)-1-(2-cyano-3-(4-((S)-1-hydroxyethyl)piperidin-1-yl)-7-methylquinoxalin-5-yl)ethyl)amino)picolinic acid;
6-chloro-3-(((R)-1-(2-cyano-3-(4-((R)-1-methoxyethyl)piperidin-1-yl)-7-methylquinoxalin-5-yl)ethyl)amino)picolinic acid;
6-chloro-3-(((R)-1-(2-cyano-3-(4-((S)-1-methoxyethyl)piperidin-1-yl)-7-methylquinoxalin-5-yl)ethyl)amino)picolinic acid;
6-chloro-3-(((R)-1-(2-cyano-3-((S)-3-methoxypiperidin-1-yl)-7-methylquinoxalin-5-yl)ethyl)amino)picolinic acid;
6-chloro-3-(((R)-1-(2-cyano-3-((R)-3-methoxypiperidin-1-yl)-7-methylquinoxalin-5-yl)ethyl)amino)picolinic acid;
2-(((R)-1-(2-cyano-3-(4-(2-((R)-2,2-difluorocyclopropyl)acetyl)piperazin-1-yl)-7-methylquinoxalin-5-yl)ethyl)amino)benzoic acid;
(R)-6-chloro-3-((1-(2-cyano-7-methyl-3-(oxetan-3-yl)quinoxalin-5-yl)ethyl)amino)picolinic acid;
(R)-6-chloro-3-((1-(2-cyano-7-methyl-3-(4-(1-methyl-1H-imidazol-4-yl)piperazin-1-yl)quinoxalin-5-yl)ethyl)amino)picolinic acid;
(R)-6-chloro-3-((1-(2-cyano-7-methyl-3-(4-(3-methylisoxazol-5-yl)piperazin-1-yl)quinoxalin-5-yl)ethyl)amino)picolinic acid;
(R)-6-chloro-3-((1-(2-cyano-7-methyl-3-(4-(5-methylisoxazol-3-yl)piperazin-1-yl)quinoxalin-5-yl)ethyl)amino)picolinic acid;
(R)-6-chloro-3-((1-(2-cyano-7-methyl-3-(2-oxo-[1,4′-bipiperidin]-1′-yl)quinoxalin-5-yl)ethyl)amino)picolinic acid;
(R)-3-((1-(2-cyano-7-methyl-3-morpholinoquinoxalin-5-yl)ethyl)amino)picolinic acid;
(R)-3-((1-(2-cyano-3-(3,3-difluoropyrrolidin-1-yl)-7-methylquinoxalin-5-yl)ethyl)amino)picolinic acid;
(R)-6-chloro-3-((1-(2-cyano-3-(4,4-difluoropiperidin-1-yl)-7-(trifluoromethoxy)quinoxalin-5-yl)ethyl)amino)picolinic acid;
(R)-3-chloro-2-cyano-6-((1-(2-cyano-3-(4,4-difluoropiperidin-1-yl)-7-methylquinoxalin-5-yl)ethyl)amino)benzoic acid;
(R)-2-cyano-6-((1-(2-cyano-3-(4,4-difluoropiperidin-1-yl)-7-methylquinoxalin-5-yl)ethyl)amino)benzoic acid;
3-(((1R)-1-(2-cyano-3-(5,5-difluoro-2-azabicyclo[2.2.1]heptan-2-yl)-7-methylquinoxalin-5-yl)ethyl)amino)picolinic acid;
(R)-3-((1-(2-cyano-7-methyl-3-(2-oxa-6-azaspiro[3.3]heptan-6-yl)quinoxalin-5-yl)ethyl)amino)picolinic acid;
6-chloro-3-(((R)-1-(2-cyano-3-((R)-3,3-difluoro-4-(hydroxymethyl)pyrrolidin-1-yl)-7-methylquinoxalin-5-yl)ethyl)amino)picolinic acid;
6-chloro-3-(((R)-1-(2-cyano-3-((S)-3,3-difluoro-4-(hydroxymethyl)pyrrolidin-1-yl)-7-methylquinoxalin-5-yl)ethyl)amino)picolinic acid;
6-chloro-3-(((R)-1-(2-cyano-3-((S)-3,3-difluoro-4-(methoxymethyl)pyrrolidin-1-yl)-7-methylquinoxalin-5-yl)ethyl)amino)picolinic acid;
6-chloro-3-(((R)-1-(2-cyano-3-((R)-3,3-difluoro-4-(methoxymethyl)pyrrolidin-1-yl)-7-methylquinoxalin-5-yl)ethyl)amino)picolinic acid;
6-chloro-3-(((R)-1-(2-cyano-3-((1S,4R)-2,2-difluoro-7-azabicyclo[2.2.1]heptan-7-yl)-7-methylquinoxalin-5-yl)ethyl)amino)picolinic acid;
6-chloro-3-(((R)-1-(2-cyano-3-((1R,4S)-2,2-difluoro-7-azabicyclo[2.2.1]heptan-7-yl)-7-methylquinoxalin-5-yl)ethyl)amino)picolinic acid;
6-chloro-3-(((R)-1-(2-cyano-3-((1R,5S,6S)-6-(methoxymethyl)-3-azabicyclo[3.1.0]hexan-3-yl)-7-methylquinoxalin-5-yl)ethyl)amino)picolinic acid;
6-chloro-3-(((1R)-1-(2-cyano-3-(6-methoxy-3-azabicyclo[3.1.0]hexan-3-yl)-7-methylquinoxalin-5-yl)ethyl)amino)picolinic acid;
6-chloro-3-(((1R)-1-(2-cyano-3-(6-fluoro-3-azabicyclo[3.1.0]hexan-3-yl)-7-methylquinoxalin-5-yl)ethyl)amino)picolinic acid;
(R)-6-chloro-3-((1-(2-cyano-3-(3,4-dihydro-2,6-naphthyridin-2(1H)-yl)-7-methylquinoxalin-5-yl)ethyl)amino)picolinic acid;
(R)-6-chloro-3-((1-(2-cyano-3-(7-cyano-3,4-dihydroisoquinolin-2(1H)-yl)-7-methylquinoxalin-5-yl)ethyl)amino)picolinic acid;
(R)-6-chloro-3-((1-(2-cyano-3-(2-oxa-6-azaspiro[3.3]heptan-6-yl)-7-(trifluoromethyl)quinoxalin-5-yl)ethyl)amino)picolinic acid;
(R)-6-chloro-3-((1-(2-cyano-3-(3,3-difluoropyrrolidin-1-yl)-7-(trifluoromethyl)quinoxalin-5-yl)ethyl)amino)picolinic acid;
6-chloro-3-(((R)-1-(2-cyano-3-((3R,5R)-3,5-difluoropiperidin-1-yl)-7-methylquinoxalin-5-yl)ethyl)amino)picolinic acid;
(R)-6-chloro-3-((1-(2-cyano-7-methyl-3-(6-methyl-5-oxo-3,4,5,6-tetrahydro-2,6-naphthyridin-2(1H)-yl)quinoxalin-5-yl)ethyl)amino)picolinic acid;
(R)-6-chloro-3-((1-(2-cyano-7-methyl-3-(7-methyl-8-oxo-3,4,7,8-tetrahydro-2,7-naphthyridin-2(1H)-yl)quinoxalin-5-yl)ethyl)amino)picolinic acid;
(R)-6-chloro-3-((1-(2-cyano-7-methyl-3-(1-methyl-6-oxo-1,6-dihydropyridin-3-yl)quinoxalin-5-yl)ethyl)amino)picolinic acid;
6-chloro-3-(((1R)-1-(2-cyano-7-methyl-3-(1-methyl-6-oxopiperidin-3-yl)quinoxalin-5-yl)ethyl)amino)picolinic acid;
(R)-6-chloro-3-((1-(2-cyano-7-methyl-3-(3-oxopyrrolidin-1-yl)quinoxalin-5-yl)ethyl)amino)picolinic acid;
6-chloro-3-(((1R)-1-(2-cyano-7-methyl-3-(3-methyl-4-oxopyrrolidin-1-yl)quinoxalin-5-yl)ethyl)amino)picolinic acid;
(R)-6-chloro-3-((1-(2-cyano-3-(3,3-dimethyl-4-oxopyrrolidin-1-yl)-7-methylquinoxalin-5-yl)ethyl)amino)picolinic acid;
6-chloro-3-(((1R)-1-(2-cyano-3-(2,2-dioxidotetrahydro-1H-thieno[3,4-c]pyrrol-5(3H)-yl)-7-methylquinoxalin-5-yl)ethyl)amino)picolinic acid;
6-chloro-3-(((R)-1-(2-cyano-7-methyl-3-((3aS,6aS)-tetrahydro-1H-furo[3,4-c]pyrrol-5(3H)-yl)quinoxalin-5-yl)ethyl)amino)picolinic acid;
6-chloro-3-(((R)-1-(2-cyano-7-methyl-3-((3aR,6aR)-tetrahydro-1H-furo[3,4-c]pyrrol-5(3H)-yl)quinoxalin-5-yl)ethyl)amino)picolinic acid;
(R)-6-chloro-3-((1-(2-cyano-3-morpholino-7-(trifluoromethyl)quinoxalin-5-yl)ethyl)amino)picolinic acid;
(R)-6-chloro-3-((1-(2-cyano-7-(difluoromethyl)-3-morpholinoquinoxalin-5-yl)ethyl)amino)picolinic acid;
(R)-6-chloro-3-((1-(2-cyano-7-ethoxy-3-morpholinoquinoxalin-5-yl)ethyl)amino)picolinic acid;
(R)-6-chloro-3-((1-(2-cyano-7-methyl-3-(tetrahydro-2H-pyran-4-yl)quinoxalin-5-yl)ethyl)amino)picolinic acid;
6-chloro-3-(((R)-1-(2-cyano-3-((R)-4,4-difluoro-3-(hydroxymethyl)piperidin-1-yl)-7-methylquinoxalin-5-yl)ethyl)amino)picolinic acid;
6-chloro-3-(((R)-1-(2-cyano-3-((S)-4,4-difluoro-3-(hydroxymethyl)piperidin-1-yl)-7-methylquinoxalin-5-yl)ethyl)amino)picolinic acid;
6-chloro-3-(((R)-1-(2-cyano-3-((S)-2-(hydroxymethyl)morpholino)-7-methylquinoxalin-5-yl)ethyl)amino)picolinic acid;
6-chloro-3-(((R)-1-(2-cyano-3-((R)-2-(hydroxymethyl)morpholino)-7-methylquinoxalin-5-yl)ethyl)amino)picolinic acid;
6-chloro-3-(((R)-1-(2-cyano-3-((S)-2-(methoxymethyl)morpholino)-7-methylquinoxalin-5-yl)ethyl)amino)picolinic acid;
6-chloro-3-(((R)-1-(2-cyano-3-((R)-2-(methoxymethyl)morpholino)-7-methylquinoxalin-5-yl)ethyl)amino)picolinic acid;
6-chloro-3-(((R)-1-(2-cyano-3-((2R,5R)-2,5-dimethylmorpholino)-7-methylquinoxalin-5-yl)ethyl)amino)picolinic acid;
6-chloro-3-(((R)-1-(2-cyano-3-((2S,5S)-2,5-dimethylmorpholino)-7-methylquinoxalin-5-yl)ethyl)amino)picolinic acid;
6-chloro-3-(((R)-1-(2-cyano-3-((2S,5R)-2,5-dimethylmorpholino)-7-methylquinoxalin-5-yl)ethyl)amino)picolinic acid;
6-chloro-3-(((R)-1-(2-cyano-3-((2R,5S)-2,5-dimethylmorpholino)-7-methylquinoxalin-5-yl)ethyl)amino)picolinic acid;
3-(((R)-1-(3-((1R,6S)-2-oxa-5-azabicyclo[4.1.0]heptan-5-yl)-2-cyano-7-methylquinoxalin-5-yl)ethyl)amino)-6-chloropicolinic acid;
3-(((R)-1-(3-((1S,6R)-2-oxa-5-azabicyclo[4.1.0]heptan-5-yl)-2-cyano-7-methylquinoxalin-5-yl)ethyl)amino)-6-chloropicolinic acid;
(R)-6-chloro-3-((1-(2-cyano-7-methyl-3-(2-oxa-6-azaspiro[3.4]octan-6-yl)quinoxalin-5-yl)ethyl)amino)picolinic acid;
3-(((R)-1-(3-((1R,6S)-3-oxabicyclo[4.1.0]heptan-6-yl)-2-cyano-7-methylquinoxalin-5-yl)ethyl)amino)-6-chloropicolinic acid;
3-(((R)-1-(3-((1S,6R)-3-oxabicyclo[4.1.0]heptan-6-yl)-2-cyano-7-methylquinoxalin-5-yl)ethyl)amino)-6-chloropicolinic acid;
(R)-6-chloro-3-((1-(2-cyano-7-methyl-3-(1,4-oxazepan-4-yl)quinoxalin-5-yl)ethyl)amino)picolinic acid;
3-(((R)-1-(3-((1R,5S)-6-oxa-3-azabicyclo[3.2.1]octan-3-yl)-2-cyano-7-methylquinoxalin-5-yl)ethyl)amino)-6-chloropicolinic acid;
3-(((R)-1-(3-((1S,5R)-6-oxa-3-azabicyclo[3.2.1]octan-3-yl)-2-cyano-7-methylquinoxalin-5-yl)ethyl)amino)-6-chloropicolinic acid;
6-chloro-3-(((1R)-1-(2-cyano-3-((1S)-1-fluoro-3-azabicyclo[3.2.0]heptan-3-yl)-7-methylquinoxalin-5-yl)ethyl)amino)picolinic acid;
6-chloro-3-(((1R)-1-(2-cyano-3-((1S)-1-fluoro-3-azabicyclo[3.2.0]heptan-3-yl)-7-methylquinoxalin-5-yl)ethyl)amino)picolinic acid;
6-chloro-3-(((R)-1-(2-cyano-3-((3S,4S)-3-fluoro-4-methylpyrrolidin-1-yl)-7-methylquinoxalin-5-yl)ethyl)amino)picolinic acid;
6-chloro-3-(((R)-1-(2-cyano-3-((2R,4S)-4-fluoro-2-methylpyrrolidin-1-yl)-7-methylquinoxalin-5-yl)ethyl)amino)picolinic acid;
6-chloro-3-(((R)-1-(2-cyano-3-((2S,4R)-4-fluoro-2-methylpyrrolidin-1-yl)-7-methylquinoxalin-5-yl)ethyl)amino)picolinic acid;
6-chloro-3-(((R)-1-(2-cyano-3-((2R,4R)-4-fluoro-2-methylpyrrolidin-1-yl)-7-methylquinoxalin-5-yl)ethyl)amino)picolinic acid;
6-chloro-3-(((R)-1-(2-cyano-3-((2S,4S)-4-fluoro-2-methylpyrrolidin-1-yl)-7-methylquinoxalin-5-yl)ethyl)amino)picolinic acid;
6-chloro-3-(((1R)-1-(2-cyano-3-((1S)-1-fluoro-3-azabicyclo[3.1.0]hexan-3-yl)-7-methylquinoxalin-5-yl)ethyl)amino)picolinic acid;
6-chloro-3-(((1R)-1-(2-cyano-3-((1R)-1-fluoro-3-azabicyclo[3.1.0]hexan-3-yl)-7-methylquinoxalin-5-yl)ethyl)amino)picolinic acid;
6-chloro-3-(((R)-1-(2-cyano-3-((R)-3,3-difluorocyclopentyl)-7-methylquinoxalin-5-yl)ethyl)amino)picolinic acid;
6-chloro-3-(((R)-1-(2-cyano-3-((S)-3,3-difluorocyclopentyl)-7-methylquinoxalin-5-yl)ethyl)amino)picolinic acid;
(R)-6-chloro-3-((1-(2-cyano-7-methyl-3-(2-oxa-7-azaspiro[3.5]nonan-7-yl)quinoxalin-5-yl)ethyl)amino)picolinic acid;
(R)-6-chloro-3-((1-(2-cyano-7-methyl-3-(1-oxa-7-azaspiro[3.5]nonan-7-yl)quinoxalin-5-yl)ethyl)amino)picolinic acid;
(R)-6-chloro-3-((1-(2-cyano-7-methyl-3-(6-oxa-2-azaspiro[3.4]octan-2-yl)quinoxalin-5-yl)ethyl)amino)picolinic acid;
(R)-5-(1-((6-chloro-2-(hydroxymethoxy)pyridin-3-yl)amino)ethyl)-7-methyl-3-(5-oxa-2-azaspiro[3.4]octan-2-yl)quinoxaline-2-carbonitrile
6-chloro-3-(((R)-1-(2-cyano-7-methyl-3-((S)-1-oxa-7-azaspiro[4.4]nonan-7-yl)quinoxalin-5-yl)ethyl)amino)picolinic acid;
6-chloro-3-(((R)-1-(2-cyano-7-methyl-3-((R)-1-oxa-7-azaspiro[4.4]nonan-7-yl)quinoxalin-5-yl)ethyl)amino)picolinic acid;
6-chloro-3-(((R)-1-(2-cyano-7-methyl-3-((S)-2-oxa-7-azaspiro[4.4]nonan-7-yl)quinoxalin-5-yl)ethyl)amino)picolinic acid;
6-chloro-3-(((R)-1-(2-cyano-7-methyl-3-((R)-2-oxa-7-azaspiro[4.4]nonan-7-yl)quinoxalin-5-yl)ethyl)amino)picolinic acid;
(R)-6-chloro-3-((1-(2-cyano-3-(6,6-difluoro-2-azaspiro[3.3]heptan-2-yl)-7-methylquinoxalin-5-yl)ethyl)amino)picolinic acid;
(R)-6-chloro-3-((1-(2-cyano-3-(5,5-difluoro-2-azaspiro[3.3]heptan-2-yl)-7-methylquinoxalin-5-yl)ethyl)amino)picolinic acid;
6-chloro-3-(((R)-1-(2-cyano-3-((S)-5-fluoro-2-azaspiro[3.3]heptan-2-yl)-7-methylquinoxalin-5-yl)ethyl)amino)picolinic acid;
6-chloro-3-(((R)-1-(2-cyano-3-((R)-5-fluoro-2-azaspiro[3.3]heptan-2-yl)-7-methylquinoxalin-5-yl)ethyl)amino)picolinic acid;
(R)-6-chloro-3-((1-(2-cyano-3-(6-fluoro-2-azaspiro[3.3]heptan-2-yl)-7-methylquinoxalin-5-yl)ethyl)amino)picolinic acid;
(R)-6-chloro-3-((1-(2-cyano-3-(6-hydroxy-2-azaspiro[3.3]heptan-2-yl)-7-methylquinoxalin-5-yl)ethyl)amino)picolinic acid;
6-chloro-3-(((R)-1-(2-cyano-7-methyl-3-(( )-2-methylmorpholino)quinoxalin-5-yl)ethyl)amino)picolinic acid;
6-chloro-3-(((R)-1-(2-cyano-3-((S)-3-((S)-1-hydroxyethyl)piperidin-1-yl)-7-methylquinoxalin-5-yl)ethyl)amino)picolinic acid;
6-chloro-3-(((R)-1-(2-cyano-3-((S)-3-((R)-1-hydroxyethyl)piperidin-1-yl)-7-methylquinoxalin-5-yl)ethyl)amino)picolinic acid;
6-chloro-3-(((R)-1-(2-cyano-3-((R)-3-((S)-1-hydroxyethyl)piperidin-1-yl)-7-methylquinoxalin-5-yl)ethyl)amino)picolinic acid;
6-chloro-3-(((R)-1-(2-cyano-3-((R)-3-((R)-1-hydroxyethyl)piperidin-1-yl)-7-methylquinoxalin-5-yl)ethyl)amino)picolinic acid;
(R)-6-chloro-3-((1-(2-cyano-3-(4,4-difluoroazepan-1-yl)-7-methylquinoxalin-5-yl)ethyl)amino)picolinic acid;
6-chloro-3-(((R)-1-(2-cyano-7-methyl-3-cis-tetrahydro-1 h-thieno[3,4-c]pyrrol-5(3H)-ylquinoxalin-5-yl)ethyl)amino)picolinic acid;
(R)-6-chloro-3-((1-(2,7-dimethyl-3-morpholinoquinoxalin-5-yl)ethyl)amino)picolinic acid;
(R)-6-chloro-3-((1-(7-methyl-3-morpholinoquinoxalin-5-yl)ethyl)amino)picolinic acid;
(R)-6-chloro-3-((1-(7-methyl-3-morpholino-2-(trifluoromethyl)quinoxalin-5-yl)ethyl)amino)picolinic acid;
(R)-5-(1-((2-(1H-tetrazol-5-yl)phenyl)amino)ethyl)-7-methyl-3-morpholinoquinoxaline-2-carbonitrile;
(R)-5-(1-((2-cyanophenyl)amino)ethyl)-7-methyl-3-morpholinoquinoxaline-2-carbonitrile;
(R)-5-(1-((6-chloro-2-(1H-tetrazol-5-yl)pyridin-3-yl)amino)ethyl)-7-methyl-3-morpholinoquinoxaline-2-carbonitrile;
(R)-5-(1-((6-chloro-2-cyanopyridin-3-yl)amino)ethyl)-7-methyl-3-morpholinoquinoxaline-2-carbonitrile;
(R)-2-((1-(2-cyano-7-methyl-3-morpholinoquinoxalin-5-yl)ethyl)amino)-N-methylbenzamide;
(R)-6-chloro-3-((1-(2-cyano-7-methyl-3-morpholinoquinoxalin-5-yl)ethyl)amino)-N-methylpicolinamide;
(R)-6-chloro-3-((1-(2-cyano-7-methyl-3-(morpholino-d8)quinoxalin-5-yl)ethyl)amino)picolinic acid;
(2S,3S,4S,5R)-6-((6-chloro-3-(((R)-1-(2-cyano-7-methyl-3-morpholinoquinoxalin-5-yl)ethyl)amino)picolinoyl)oxy)-3,4,5-trihydroxytetrahydro-2H-pyran-2-carboxylic acid; or
a pharmaceutically acceptable salt thereof.

67. A Compound according to claim 1 which is
(R)-2-((1-(3-(4-(bicyclo[1.1.1]pentan-1-yl-3-d)piperidin-1-yl)-2-cyano-7-methylquinoxalin-5-yl)ethyl)amino)benzoic acid;
(R)-2-((1-(2-cyano-3-((3-cyanophenyl)amino)-7-methylquinoxalin-5-yl)ethyl)amino)benzoic acid;
2-(((1R)-1-(2-cyano-7-methyl-3-(((tetrahydrofuran-3-yl)methyl)amino)quinoxalin-5-yl)ethyl)amino)benzoic acid;
(R)-2-((1-(2-cyano-7-methyl-3-((oxetan-3-ylmethyl)amino)quinoxalin-5-yl)ethyl)amino)benzoic acid;
(R)-2-((1-(2-cyano-3-((2-cyanoallyl)amino)-7-methylquinoxalin-5-yl)ethyl)amino)benzoic acid;
(R)-2-((1-(2-cyano-3-(cyclobutylamino)-7-methylquinoxalin-5-yl)ethyl)amino)benzoic acid;
(R)-2-((1-(2-cyano-3-((3-(hydroxymethyl)bicyclo[1.1.1]pentan-1-yl)amino)-7-methylquinoxalin-5-yl)ethyl)amino)benzoic acid;
(R)-2-((1-(2-cyano-3-(5,6-dihydroimidazo[1,2-a]pyrazin-7(8H)-yl)-7-methylquinoxalin-5-yl)ethyl)amino)benzoic acid;
(R)-2-((1-(2-cyano-3-(2,3-dimethyl-5,6-dihydroimidazo[1,2-a]pyrazin-7(8H)-yl)-7-methylquinoxalin-5-yl)ethyl)amino)benzoic acid;
(R)-2-((1-(2-cyano-3-(5,6-dihydroimidazo[1,5-a]pyrazin-7(8H)-yl)-7-methylquinoxalin-5-yl)ethyl)amino)benzoic acid;
(R)-2-((1-(2-cyano-3-(6,7-dihydropyrazolo[1,5-a]pyrazin-5(4H)-yl)-7-methylquinoxalin-5-yl)ethyl)amino)benzoic acid;
(R)-2-((1-(2-cyano-7-methyl-3-(piperazin-1-yl)quinoxalin-5-yl)ethyl)amino)benzoic acid;
(R)-2-((1-(2-cyano-7-methyl-3-(4-methylpiperazin-1-yl)quinoxalin-5-yl)ethyl)amino)benzoic acid;
(R)-2-((1-(3-(azepan-1-yl)-2-cyano-7-methylquinoxalin-5-yl)ethyl)amino)benzoic acid;
(R)-2-((1-(2-cyano-7-methyl-3-(2-oxa-8-azaspiro[4.5]decan-8-yl)quinoxalin-5-yl)ethyl)amino)benzoic acid;
(R)-2-((1-(2-cyano-7-methyl-3-(2-oxa-7-azaspiro[3.5]nonan-7-yl)quinoxalin-5-yl)ethyl)amino)benzoic acid;
(R)-2-((1-(2-cyano-7-methyl-3-(6-azaspiro[2.5]octan-6-yl)quinoxalin-5-yl)ethyl)amino)benzoic acid;
(R)-2-((1-(3-(azetidin-1-yl)-2-cyano-7-methylquinoxalin-5-yl)ethyl)amino)benzoic acid;
2-(((1R)-1-(2-cyano-3-(2-hydroxy-8-azaspiro[4.5]decan-8-yl)-7-methylquinoxalin-5-yl)ethyl)amino)benzoic acid;
(R)-2-((1-(2-cyano-7-methyl-3-(4-methylpiperidin-1-yl)quinoxalin-5-yl)ethyl)amino)benzoic acid;
2-(((1R)-1-(2-cyano-7-methyl-3-(octahydro-2H-4,7-ethanoisoindol-2-yl)quinoxalin-5-yl)ethyl)amino)benzoic acid;
2-(((1R)-1-(2-cyano-3-(6,6-dimethyl-3-azabicyclo[3.2.0]heptan-3-yl)-7-methylquinoxalin-5-yl)ethyl)amino)benzoic acid;
(R)-2-((1-(2-cyano-7-methyl-3-(2-azaspiro[3.3]heptan-2-yl)quinoxalin-5-yl)ethyl)amino)benzoic acid;
(R)-2-((1-(2-cyano-3-(2-azadispiro[3.1.36.14]decan-2-yl)-7-methylquinoxalin-5-yl)ethyl)amino)benzoic acid;
(R)-2-((1-(2-cyano-7-methyl-3-(4-oxa-7-azaspiro[2.5]octan-7-yl)quinoxalin-5-yl)ethyl)amino)benzoic acid;
2-(((1R)-1-(2-cyano-7-methyl-3-(5-methylhexahydropyrrolo[3,4-c]pyrrol-2(1H)-yl)quinoxalin-5-yl)ethyl)amino)benzoic acid;
2-(((1R)-1-(2-cyano-3-(2-cyclopropylmorpholino)-7-methylquinoxalin-5-yl)ethyl)amino)benzoic acid;
2-(((1R)-1-(2-cyano-3-(2-cyclopropyl-6-methylmorpholino)-7-methylquinoxalin-5-yl)ethyl)amino)benzoic acid;
2-(((1R)-1-(3-(6-oxa-3-azabicyclo[3.1.1]heptan-3-yl)-2-cyano-7-methylquinoxalin-5-yl)ethyl)amino)benzoic acid;
(R)-2-((1-(3-(3-azabicyclo[3.1.1]heptan-3-yl)-2-cyano-7-methylquinoxalin-5-yl)ethyl)amino)benzoic acid;
2-(((1R)-1-(3-(3-azabicyclo[3.1.0]hexan-3-yl)-2-cyano-7-methylquinoxalin-5-yl)ethyl)amino)benzoic acid;
(R)-2-((1-(2-cyano-3-(6-(4-cyanophenyl)-2,6-diazaspiro[3.3]heptan-2-yl)-7-methylquinoxalin-5-yl)ethyl)amino)benzoic acid;
2-(((1R)-1-(2-cyano-7-methyl-3-(2-methylmorpholino)quinoxalin-5-yl)ethyl)amino)benzoic acid;
2-(((1R)-1-(2-cyano-3-(2-cyclopropyl-2-methylmorpholino)-7-methylquinoxalin-5-yl)ethyl)amino)benzoic acid;
2-(((1R)-1-(3-(3,6-diazabicyclo[3.1.1]heptan-6-yl)-2-cyano-7-methylquinoxalin-5-yl)ethyl)amino)benzoic acid;
(R)-2-((1-(3-(7-azabicyclo[2.2.1]heptan-7-yl)-2-cyano-7-methylquinoxalin-5-yl)ethyl)amino)benzoic acid;
(R)-2-((1-(3-(2-azaadamantan-2-yl)-2-cyano-7-methylquinoxalin-5-yl)ethyl)amino)benzoic acid;
(R)-2-((1-(2-cyano-3-(1-(methoxymethyl)-4-methyl-2-azabicyclo[2.1.1]hexan-2-yl)-7-methylquinoxalin-5-yl)ethyl)amino)benzoic acid;
2-(((1R)-1-(2-cyano-3-(8-((dimethylamino)methyl)-6-oxa-9-azaspiro[4.5]decan-9-yl)-7-methylquinoxalin-5-yl)ethyl)amino)benzoic acid;
(R)-2-((1-(3-(2-azabicyclo[2.2.2]octan-2-yl)-2-cyano-7-methylquinoxalin-5-yl)ethyl)amino)benzoic acid;
2-(((1R)-1-(3-(2-azabicyclo[2.2.1]heptan-2-yl)-2-cyano-7-methylquinoxalin-5-yl)ethyl)amino)benzoic acid;
2-(((1R)-1-(2-cyano-3-(hexahydro-3,5-methanocyclopenta[b]pyrrol-1(2H)-yl)-7-methylquinoxalin-5-yl)ethyl)amino)benzoic acid;
2-(((1R)-1-(2-cyano-3-(1-(hydroxymethyl)-6-azabicyclo[3.2.1]octan-6-yl)-7-methylquinoxalin-5-yl)ethyl)amino)benzoic acid;
(R)-2-((1-(3-(2-azabicyclo[2.1.1]hexan-2-yl)-2-cyano-7-methylquinoxalin-5-yl)ethyl)amino)benzoic acid;
2-(((1R)-1-(2-cyano-7-methyl-3-(3-methylmorpholino)quinoxalin-5-yl)ethyl)amino)benzoic acid;
2-(((1R)-1-(2-cyano-7-methyl-3-(2-methyl-4-(pyridin-3-ylmethyl)piperazin-1-yl)quinoxalin-5-yl)ethyl)amino)benzoic acid;
2-(((1R)-1-(2-cyano-7-methyl-3-(2-methylazetidin-1-yl)quinoxalin-5-yl)ethyl)amino)benzoic acid;
2-(((1R)-1-(2-cyano-3-(2,3-dimethylmorpholino)-7-methylquinoxalin-5-yl)ethyl)amino)benzoic acid;
2-(((1R)-1-(2-cyano-3-(3-hydroxy-2-(2-methoxyphenyl)pyrrolidin-1-yl)-7-methylquinoxalin-5-yl)ethyl)amino)benzoic acid;
(R)-2-((1-(2-cyano-3-(3,4-dihydroquinolin-1(2H)-yl)-7-methylquinoxalin-5-yl)ethyl)amino)benzoic acid;
2-(((1R)-1-(2-cyano-3-(3-hydroxypiperidin-1-yl)-7-methylquinoxalin-5-yl)ethyl)amino)benzoic acid;
2-(((1R)-1-(2-cyano-3-(3-hydroxy-3-methylpiperidin-1-yl)-7-methylquinoxalin-5-yl)ethyl)amino)benzoic acid;
2-(((1R)-1-(2-cyano-3-(2-(4-hydroxy-4-methyltetrahydro-2H-pyran-3-yl)pyrrolidin-1-yl)-7-methylquinoxalin-5-yl)ethyl)amino)benzoic acid;
2-(((1R)-1-(2-cyano-3-(8-hydroxy-3-azabicyclo[3.2.1]octan-3-yl)-7-methylquinoxalin-5-yl)ethyl)amino)benzoic acid;
2-(((1R)-1-(2-cyano-3-(5-hydroxy-2-azabicyclo[2.2.2]octan-2-yl)-7-methylquinoxalin-5-yl)ethyl)amino)benzoic acid;
2-(((1R)-1-(2-cyano-3-(6-hydroxyhexahydro-3,5-methanocyclopenta[b]pyrrol-1(2H)-yl)-7-methylquinoxalin-5-yl)ethyl)amino)benzoic acid;
2-(((1R)-1-(2-cyano-3-(6-(4-cyanophenyl)-3,6-diazabicyclo[3.1.1]heptan-3-yl)-7-methylquinoxalin-5-yl)ethyl)amino)benzoic acid;
2-(((1R)-1-(2-cyano-3-(6-hydroxy-6-methyl-3-azabicyclo[3.1.1]heptan-3-yl)-7-methylquinoxalin-5-yl)ethyl)amino)benzoic acid;
2-(((1R)-1-(2-cyano-3-(5-(4-cyanophenyl)-2,5-diazabicyclo[2.2.1]heptan-2-yl)-7-methylquinoxalin-5-yl)ethyl)amino)benzoic acid;
2-(((1R)-1-(2-cyano-7-methyl-3-(5-phenyl-2-azabicyclo[2.2.1]heptan-2-yl)quinoxalin-5-yl)ethyl)amino)benzoic acid;
2-(((1R)-1-(3-(7-(tert-butyl)-6,7-dihydropyrazolo[1,5-a]pyrimidin-4(5H)-yl)-2-cyano-7-methylquinoxalin-5-yl)ethyl)amino)benzoic acid;
(R)-2-((1-(2-cyano-7-methyl-3-(1-methyl-1,4,5,7-tetrahydro-6H-pyrazolo[3,4-c]pyridin-6-yl)quinoxalin-5-yl)ethyl)amino)benzoic acid;
(R)-2-((1-(2-cyano-7-methyl-3-(5,6,8,9-tetrahydro-7H-imidazo[1,2-d][1,4]diazepin-7-yl)quinoxalin-5-yl)ethyl)amino)benzoic acid;
(R)-2-((1-(2-cyano-7-methyl-3-(4-(pyridin-2-yl)piperazin-1-yl)quinoxalin-5-yl)ethyl)amino)benzoic acid;
(R)-2-((1-(2-cyano-7-methyl-3-(4-(pyridin-3-yl)piperazin-1-yl)quinoxalin-5-yl)ethyl)amino)benzoic acid;
(R)-2-((1-(2-cyano-3-(4-(3-cyano-1-methyl-1H-pyrazol-4-yl)piperazin-1-yl)-7-methylquinoxalin-5-yl)ethyl)amino)benzoic acid;
(R)-2-((1-(2-cyano-7-methyl-3-(4-(1-methyl-1H-pyrazol-4-yl)piperazin-1-yl)quinoxalin-5-yl)ethyl)amino)benzoic acid;
(R)-2-((1-(2-cyano-7-methyl-3-(4-(pyridin-4-yl)piperazin-1-yl)quinoxalin-5-yl)ethyl)amino)benzoic acid;
(R)-2-((1-(2-cyano-7-methyl-3-(4-phenylpiperazin-1-yl)quinoxalin-5-yl)ethyl)amino)benzoic acid;
(R)-2-((1-(3-(4-(4-(aminomethyl)phenyl)piperazin-1-yl)-2-cyano-7-methylquinoxalin-5-yl)ethyl)amino)benzoic acid;
(R)-2-((1-(2-cyano-7-methyl-3-(4-(3-methylisoxazol-5-yl)piperazin-1-yl)quinoxalin-5-yl)ethyl)amino)benzoic acid;
(R)-2-((1-(2-cyano-3-(4-(3-cyanophenyl)piperazin-1-yl)-7-methylquinoxalin-5-yl)ethyl)amino)benzoic acid;
(R)-2-((1-(2-cyano-3-(4-(2,6-dimethylphenyl)piperazin-1-yl)-7-methylquinoxalin-5-yl)ethyl)amino)benzoic acid;
(R)-2-((1-(2-cyano-7-methyl-3-(4-(o-tolyl)piperazin-1-yl)quinoxalin-5-yl)ethyl)amino)benzoic acid;
(R)-2-((1-(2-cyano-3-(4-(3-cyano-2-methylphenyl)piperazin-1-yl)-7-methylquinoxalin-5-yl)ethyl)amino)benzoic acid;
(R)-2-((1-(2-cyano-3-(4-(2-cyanopyridin-3-yl)piperazin-1-yl)-7-methylquinoxalin-5-yl)ethyl)amino)benzoic acid;
(R)-2-((1-(2-cyano-3-(4-(5-cyanopyrimidin-2-yl)piperazin-1-yl)-7-methylquinoxalin-5-yl)ethyl)amino)benzoic acid;
(R)-2-((1-(2-cyano-3-(4-(3-cyano-1-methyl-1H-indazol-7-yl)piperazin-1-yl)-7-methylquinoxalin-5-yl)ethyl)amino)benzoic acid;
(R)-2-((1-(2-cyano-3-(4-(2-cyanophenyl)piperazin-1-yl)-7-methylquinoxalin-5-yl)ethyl)amino)benzoic acid;
(R)-2-((1-(2-cyano-7-methyl-3-(4-(4-(methylcarbamoyl)phenyl)piperazin-1-yl)quinoxalin-5-yl)ethyl)amino)benzoic acid;
(R)-2-((1-(3-(4-(4-carbamoylphenyl)piperazin-1-yl)-2-cyano-7-methylquinoxalin-5-yl)ethyl)amino)benzoic acid;
(R)-2-((1-(2-cyano-3-(4-(4-cyanophenyl)piperazin-1-yl)-7-methylquinoxalin-5-yl)ethyl)amino)benzoic acid;
(R)-2-((1-(2-cyano-3-(4-(4-cyano-2-methylphenyl)piperazin-1-yl)-7-methylquinoxalin-5-yl)ethyl)amino)benzoic acid;
(R)-2-((1-(2-cyano-3-(4-(8-cyanoisoquinolin-5-yl)piperazin-1-yl)-7-methylquinoxalin-5-yl)ethyl)amino)benzoic acid;
(R)-2-((1-(2-cyano-3-(4-(4-cyano-7-hydroxynaphthalen-1-yl)piperazin-1-yl)-7-methylquinoxalin-5-yl)ethyl)amino)benzoic acid;
(R)-2-((1-(2-cyano-3-(4-(4-cyano-6-hydroxynaphthalen-1-yl)piperazin-1-yl)-7-methylquinoxalin-5-yl)ethyl)amino)benzoic acid;
(R)-2-((1-(2-cyano-3-(4-(4,6-dicyanonaphthalen-1-yl)piperazin-1-yl)-7-methylquinoxalin-5-yl)ethyl)amino)benzoic acid;
(R)-2-((1-(2-cyano-7-methyl-3-(4-(oxetan-3-yl)piperazin-1-yl)quinoxalin-5-yl)ethyl)amino)benzoic acid;
(R)-2-((1-(2-cyano-7-methyl-3-(4-(tetrahydro-2H-pyran-4-yl)piperazin-1-yl)quinoxalin-5-yl)ethyl)amino)benzoic acid;
(R)-2-((1-(2-cyano-3-(4-cyclopropylpiperazin-1-yl)-7-methylquinoxalin-5-yl)ethyl)amino)benzoic acid;
(R)-2-((1-(2-cyano-7-methyl-3-(4-(1-methyl-1H-pyrazole-4-carbonyl)piperazin-1-yl)quinoxalin-5-yl)ethyl)amino)benzoic acid;
(R)-2-((1-(3-(4-benzoylpiperazin-1-yl)-2-cyano-7-methylquinoxalin-5-yl)ethyl)amino)benzoic acid;
(R)-2-((1-(2-cyano-3-(4-(cyclopentanecarbonyl)piperazin-1-yl)-7-methylquinoxalin-5-yl)ethyl)amino)benzoic acid;
(R)-2-((1-(2-cyano-3-(4-(cyclopropanecarbonyl)piperazin-1-yl)-7-methylquinoxalin-5-yl)ethyl)amino)benzoic acid;
(R)-2-((1-(3-(4-acetylpiperazin-1-yl)-2-cyano-7-methylquinoxalin-5-yl)ethyl)amino)benzoic acid;
(R)-2-((1-(3-(4-(1H-pyrazol-1-yl)piperidin-1-yl)-2-cyano-7-methylquinoxalin-5-yl)ethyl)amino)benzoic acid;
(R)-2-((1-(2-cyano-7-methyl-3-(4-(4-methyl-1H-pyrazol-1-yl)piperidin-1-yl)quinoxalin-5-yl)ethyl)amino)benzoic acid;
(R)-2-((1-(2-cyano-7-methyl-3-(4-(1-methyl-1H-pyrazol-3-yl)piperidin-1-yl)quinoxalin-5-yl)ethyl)amino)benzoic acid;
(R)-2-((1-(3-(4-(1H-pyrazol-3-yl)piperidin-1-yl)-2-cyano-7-methylquinoxalin-5-yl)ethyl)amino)benzoic acid;
(R)-2-((1-(2-cyano-7-methyl-3-(4-(1-methyl-1H-pyrazol-5-yl)piperidin-1-yl)quinoxalin-5-yl)ethyl)amino)benzoic acid;
(R)-2-((1-(2-cyano-3-(4-(4-methoxyphenyl)piperidin-1-yl)-7-methylquinoxalin-5-yl)ethyl)amino)benzoic acid;
(R)-2-((1-(2-cyano-3-(4-methoxypiperidin-1-yl)-7-methylquinoxalin-5-yl)ethyl)amino)benzoic acid;
(R)-2-((1-(2-cyano-3-(4-hydroxypiperidin-1-yl)-7-methylquinoxalin-5-yl)ethyl)amino)benzoic acid;
(R)-2-((1-(3-([1,4′-bipiperidin]-1′-yl)-2-cyano-7-methylquinoxalin-5-yl)ethyl)amino)benzoic acid;
(R)-2-((1-(2-cyano-3-(4-(dimethylamino)piperidin-1-yl)-7-methylquinoxalin-5-yl)ethyl)amino)benzoic acid;
(R)-2-((1-(2-cyano-3-(4-cyclopentylpiperidin-1-yl)-7-methylquinoxalin-5-yl)ethyl)amino)benzoic acid;
(R)-2-((1-(2-cyano-3-(4-cyclobutylpiperidin-1-yl)-7-methylquinoxalin-5-yl)ethyl)amino)benzoic acid;
(R)-2-((1-(2-cyano-3-(4-cyanopiperidin-1-yl)-7-methylquinoxalin-5-yl)ethyl)amino)benzoic acid;
(R)-2-((1-(2-cyano-7-methyl-3-(1-oxa-8-azaspiro[4.5]decan-8-yl)quinoxalin-5-yl)ethyl)amino)benzoic acid;
(R)-2-((1-(2-cyano-7-methyl-3-(1-oxa-7-azaspiro[3.5]nonan-7-yl)quinoxalin-5-yl)ethyl)amino)benzoic acid;
(R)-2-((1-(2-cyano-3-(4-methoxy-4-methylpiperidin-1-yl)-7-methylquinoxalin-5-yl)ethyl)amino)benzoic acid;
(R)-2-((1-(2-cyano-3-(4-cyano-4-methylpiperidin-1-yl)-7-methylquinoxalin-5-yl)ethyl)amino)benzoic acid;
2-(((1R)-1-(2-cyano-7-methyl-3-(3-(pyridin-2-yl)pyrrolidin-1-yl)quinoxalin-5-yl)ethyl)amino)benzoic acid;
2-(((1R)-1-(2-cyano-7-methyl-3-(3-(pyridin-4-yl)pyrrolidin-1-yl)quinoxalin-5-yl)ethyl)amino)benzoic acid;
2-(((1R)-1-(2-cyano-7-methyl-3-(3-phenylpyrrolidin-1-yl)quinoxalin-5-yl)ethyl)amino)benzoic acid;
2-(((1R)-1-(2-cyano-3-(3-(1,3-dihydroxypropan-2-yl)pyrrolidin-1-yl)-7-methylquinoxalin-5-yl)ethyl)amino)benzoic acid;
2-(((1R)-1-(2-cyano-7-methyl-3-(1-oxa-6-azaspiro[3.4]octan-6-yl)quinoxalin-5-yl)ethyl)amino)benzoic acid;
2-(((1R)-1-(2-cyano-7-methyl-3-(2-oxa-7-azaspiro[4.4]nonan-7-yl)quinoxalin-5-yl)ethyl)amino)benzoic acid;
2-(((1R)-1-(2-cyano-7-methyl-3-(spiro[bicyclo[2.2.1]heptane-2,3′-pyrrolidin]-1′-yl)quinoxalin-5-yl)ethyl)amino)benzoic acid;
2-(((1R)-1-(2-cyano-3-(8,9-dihydroxy-6-oxa-2-azaspiro[4.5]decan-2-yl)-7-methylquinoxalin-5-yl)ethyl)amino)benzoic acid;
2-(((1R)-1-(2-cyano-3-(4-(4-cyanophenyl)-3-methylpiperazin-1-yl)-7-methylquinoxalin-5-yl)ethyl)amino)benzoic acid;
2-(((1R)-1-(2-cyano-7-methyl-3-(2-methyl-4-phenylpiperazin-1-yl)quinoxalin-5-yl)ethyl)amino)benzoic acid;
2-(((1R)-1-(2-cyano-3-(4-(3-cyanophenyl)-2-methylpiperazin-1-yl)-7-methylquinoxalin-5-yl)ethyl)amino)benzoic acid;
2-(((1R)-1-(2-cyano-3-(4-(2-cyanophenyl)-2-methylpiperazin-1-yl)-7-methylquinoxalin-5-yl)ethyl)amino)benzoic acid;
2-(((1R)-1-(2-cyano-3-(4-(4-cyanophenyl)-2-methylpiperazin-1-yl)-7-methylquinoxalin-5-yl)ethyl)amino)benzoic acid;
2-(((1R)-1-(2-cyano-3-(4-(2-cyano-5-methylphenyl)-2-methylpiperazin-1-yl)-7-methylquinoxalin-5-yl)ethyl)amino)benzoic acid;
2-(((1R)-1-(2-cyano-3-(6-hydroxy-2-azabicyclo[2.2.2]octan-2-yl)-7-methylquinoxalin-5-yl)ethyl)amino)benzoic acid;
2-(((1R)-1-(2-cyano-7-methyl-3-(1,2,3,4-tetrahydro-1,4-epiminonaphthalen-9-yl)quinoxalin-5-yl)ethyl)amino)benzoic acid;
(R)-2-((1-(2-cyano-3-(5,7-dihydro-6H-pyrrolo[3,4-b]pyrazin-6-yl)-7-methylquinoxalin-5-yl)ethyl)amino)benzoic acid;
(R)-2-((1-(2-cyano-3-(1,3-dihydro-2H-pyrrolo[3,4-c]pyridin-2-yl)-7-methylquinoxalin-5-yl)ethyl)amino)benzoic acid;
(R)-2-((1-(2-cyano-3-(5,7-dihydro-6H-pyrrolo[3,4-b]pyridin-6-yl)-7-methylquinoxalin-5-yl)ethyl)amino)benzoic acid;
(R)-2-((1-(2-cyano-3-(4-cyanoisoindolin-2-yl)-7-methylquinoxalin-5-yl)ethyl)amino)benzoic acid;
(R)-2-((1-(2-cyano-3-(5-(hydroxymethyl)isoindolin-2-yl)-7-methylquinoxalin-5-yl)ethyl)amino)benzoic acid;
(R)-2-((1-(2-cyano-7-methyl-3-(5-methylisoindolin-2-yl)quinoxalin-5-yl)ethyl)amino)benzoic acid;
(R)-2-((1-(2-cyano-3-(5,6-dimethoxyisoindolin-2-yl)-7-methylquinoxalin-5-yl)ethyl)amino)benzoic acid;
2-(((1R)-1-(2-cyano-7-methyl-3-(tetrahydro-1H,3H-spiro[pyrrolo[2,1-c][1,4]oxazine-4,5′-[1,3]oxazinan]-3′-yl)quinoxalin-5-yl)ethyl)amino)benzoic acid;
2-(((1R)-1-(2-cyano-3-(decahydro-2H-cycloocta[c]pyrrol-2-yl)-7-methylquinoxalin-5-yl)ethyl)amino)benzoic acid;
2-(((1R)-1-(2-cyano-7-methyl-3-(8-azatricyclo[4.3.0.02,5]nonan-8-yl)quinoxalin-5-yl)ethyl)amino)benzoic acid;
(R)-2-((1-(3-(1H,3H,4H-3a,6a-(methanooxymethano)furo[3,4-c]pyrrol-5(6H)-yl)-2-cyano-7-methylquinoxalin-5-yl)ethyl)amino)benzoic acid;
2-(((1R)-1-(2-cyano-3-(8-hydroxy-6-azaspiro[3.4]octan-6-yl)-7-methylquinoxalin-5-yl)ethyl)amino)benzoic acid;
(R)-2-((1-(2-cyano-7-methyl-3-(3-(pyridin-2-yl)azetidin-1-yl)quinoxalin-5-yl)ethyl)amino)benzoic acid;
(R)-2-((1-(2-cyano-7-methyl-3-(3-(pyridin-3-yl)azetidin-1-yl)quinoxalin-5-yl)ethyl)amino)benzoic acid;
(R)-2-((1-(2-cyano-7-methyl-3-(3-phenylazetidin-1-yl)quinoxalin-5-yl)ethyl)amino)benzoic acid;
(R)-2-((1-(2-cyano-3-(3-methoxyazetidin-1-yl)-7-methylquinoxalin-5-yl)ethyl)amino)benzoic acid;
(R)-2-((1-(2-cyano-3-(3-cyanoazetidin-1-yl)-7-methylquinoxalin-5-yl)ethyl)amino)benzoic acid;
(R)-2-((1-(2-cyano-3-(7-(4-cyanophenyl)-2,7-diazaspiro[3.5]nonan-2-yl)-7-methylquinoxalin-5-yl)ethyl)amino)benzoic acid;
2-(((1R)-1-(2-cyano-3-(8-hydroxyoctahydro-2H-4,7-methanoisoindol-2-yl)-7-methylquinoxalin-5-yl)ethyl)amino)benzoic acid;
2-(((1R)-1-(2-cyano-7-methyl-3-(5-methyl-2-azabicyclo[2.2.1]heptan-2-yl)quinoxalin-5-yl)ethyl)amino)benzoic acid;
2-(((1R)-1-(2-cyano-7-methyl-3-(1′-methyltetrahydrospiro[furo[3,4-b]pyrrole-3,4′-piperidin]-1(2H)-yl)quinoxalin-5-yl)ethyl)amino)benzoic acid;
2-(((1R)-1-(2-cyano-3-(1-(hydroxymethyl)isoindolin-2-yl)-7-methylquinoxalin-5-yl)ethyl)amino)benzoic acid;
2-(((1R)-1-(2-cyano-3-(3-hydroxyoctahydro-1H-indol-1-yl)-7-methylquinoxalin-5-yl)ethyl)amino)benzoic acid;
2-(((1R)-1-(2-cyano-7-methyl-3-(1-methyloctahydro-2H-isoindol-2-yl)quinoxalin-5-yl)ethyl)amino)benzoic acid;
(R)-2-((1-(2-cyano-7-methyl-3-(methyl((5,6,8,9-tetrahydro-[1,2,4]triazolo[4,3-d][1,4]oxazepin-3-yl)methyl)amino)quinoxalin-5-yl)ethyl)amino)benzoic acid;
2-(((1R)-1-(2-cyano-7-methyl-3-(methyl((5,6,7,8-tetrahydroimidazo[1,2-a]pyridin-7-yl)methyl)amino)quinoxalin-5-yl)ethyl)amino)benzoic acid;
(R)-2-((1-(2-cyano-3-((cyclopropylmethyl)(methyl)amino)-7-methylquinoxalin-5-yl)ethyl)amino)benzoic acid;
2-(((1R)-1-(2-cyano-3-(((1-hydroxy-2,3-dihydro-1H-inden-1-yl)methyl)(methyl)amino)-7-methylquinoxalin-5-yl)ethyl)amino)benzoic acid;
(R)-2-((1-(2-cyano-3-(dimethylamino)-7-methylquinoxalin-5-yl)ethyl)amino)benzoic acid;
2-(((1R)-1-(2-cyano-7-methyl-3-(methyl(1-methyl-2,3-dihydro-1H-inden-2-yl)amino)quinoxalin-5-yl)ethyl)amino)benzoic acid;
2-(((1R)-1-(2-cyano-3-((2-methoxy-2,3-dihydro-1H-inden-1-yl)(methyl)amino)-7-methylquinoxalin-5-yl)ethyl)amino)benzoic acid;
2-(((1R)-1-(2-cyano-3-(3-(4-cyanophenyl)-3,8-diazabicyclo[3.2.1]octan-8-yl)-7-methylquinoxalin-5-yl)ethyl)amino)benzoic acid;
2-(((1R)-1-(2-cyano-3-(3-(4-cyanophenyl)-3,6-diazabicyclo[3.1.1]heptan-6-yl)-7-methylquinoxalin-5-yl)ethyl)amino)benzoic acid;
(R)-2-((1-(2-cyano-7-methyl-3-(tetrahydro-2H-pyran-4-yl)quinoxalin-5-yl)ethyl)amino)benzoic acid;
(R)-2-((1-(2-cyano-7-methyl-3-(piperidin-4-yl)quinoxalin-5-yl)ethyl)amino)benzoic acid;
(R)-2-((1-(2-cyano-7-methyl-3-(1-methylpiperidin-4-yl)quinoxalin-5-yl)ethyl)amino)benzoic acid;
(R)-2-((1-(2-cyano-3-(3,6-dihydro-2H-pyran-4-yl)-7-methylquinoxalin-5-yl)ethyl)amino)benzoic acid;
(R)-2-((1-(2-cyano-7-methyl-3-(1,2,3,6-tetrahydropyridin-4-yl)quinoxalin-5-yl)ethyl)amino)benzoic acid;
(R)-2-((1-(2-cyano-7-methyl-3-(1-methyl-1,2,3,6-tetrahydropyridin-4-yl)quinoxalin-5-yl)ethyl)amino)benzoic acid;
(R)-2-((1-(2-cyano-3,7-dimethylquinoxalin-5-yl)ethyl)amino)benzoic acid;
(R)-2-((1-(2-cyano-3-(1-(4-cyanophenyl)piperidin-4-yl)-7-methylquinoxalin-5-yl)ethyl)amino)benzoic acid;
(R)-2-((1-(3-(1-acetylpiperidin-4-yl)-2-cyano-7-methylquinoxalin-5-yl)ethyl)amino)benzoic acid;
(R)-2-((1-(3-(1-acetyl-1,2,3,6-tetrahydropyridin-4-yl)-2-cyano-7-methylquinoxalin-5-yl)ethyl)amino)benzoic acid;
(R)-2-((1-(2-cyano-3-((4-cyanophenyl)ethynyl)-7-methylquinoxalin-5-yl)ethyl)amino)benzoic acid;
(R)-2-((1-(2-cyano-7-methyl-3-(1-methyl-1H-pyrazol-3-yl)quinoxalin-5-yl)ethyl)amino)benzoic acid;
(R)-2-((1-(2-cyano-7-methyl-3-(pyridin-2-yl)quinoxalin-5-yl)ethyl)amino)benzoic acid;
(R)-2-((1-(2-cyano-7-methyl-3-(pyridin-3-yl)quinoxalin-5-yl)ethyl)amino)benzoic acid;
(R)-2-((1-(2-cyano-7-methyl-3-(1-methyl-1H-pyrazol-4-yl)quinoxalin-5-yl)ethyl)amino)benzoic acid;
(R)-2-((1-(2-cyano-7-methyl-3-(1-methyl-1H-imidazol-4-yl)quinoxalin-5-yl)ethyl)amino)benzoic acid;
(R)-2-((1-(2-cyano-3-(1-(4-cyanophenyl)-1H-pyrazol-4-yl)-7-methylquinoxalin-5-yl)ethyl)amino)benzoic acid;
(R)-2-((1-(2-cyano-7-methyl-3-(pyridin-4-yl)quinoxalin-5-yl)ethyl)amino)benzoic acid;
(R)-2-((1-(2-cyano-7-methyl-3-phenylquinoxalin-5-yl)ethyl)amino)benzoic acid;
(R)-2-((1-(2-cyano-7-methyl-3-(p-tolyl)quinoxalin-5-yl)ethyl)amino)benzoic acid;
(R)-2-((1-(2-cyano-3-(3-methoxyphenyl)-7-methylquinoxalin-5-yl)ethyl)amino)benzoic acid;
(R)-2-((1-(2-cyano-3-(3-(2-cyanopropan-2-yl)phenyl)-7-methylquinoxalin-5-yl)ethyl)amino)benzoic acid;
(R)-2-((1-(2-cyano-3-(3-cyanophenyl)-7-methylquinoxalin-5-yl)ethyl)amino)benzoic acid;
(R)-2-((1-(2-cyano-7-methyl-3-(1H-pyrazol-4-yl)quinoxalin-5-yl)ethyl)amino)benzoic acid;
(R)-2-((1-(2-cyano-3-(1H-imidazol-4-yl)-7-methylquinoxalin-5-yl)ethyl)amino)benzoic acid;
(R)-2-((1-(2-cyano-7-methyl-3-(o-tolyl)quinoxalin-5-yl)ethyl)amino)benzoic acid;
(R)-2-((1-(2-cyano-3-(1-(4-cyanophenyl)-1H-imidazol-4-yl)-7-methylquinoxalin-5-yl)ethyl)amino)benzoic acid;
(R)-2-((1-(2-cyano-3-(2-methoxyphenyl)-7-methylquinoxalin-5-yl)ethyl)amino)benzoic acid;
(R)-2-((1-(2-cyano-3-(2-cyanophenyl)-7-methylquinoxalin-5-yl)ethyl)amino)benzoic acid;
(R)-2-((1-(2-cyano-3-(4-methoxyphenyl)-7-methylquinoxalin-5-yl)ethyl)amino)benzoic acid;
(R)-2-((1-(2-cyano-3-(4-cyanophenyl)-7-methylquinoxalin-5-yl)ethyl)amino)benzoic acid;
2-(((1R)-1-(2-cyano-7-methyl-3-(1-methyl-2-oxopiperidin-4-yl)quinoxalin-5-yl)ethyl)amino)benzoic acid;
(R)-2-((1-(2-cyano-3-(4,4-difluorocyclohexyl)-7-methylquinoxalin-5-yl)ethyl)amino)benzoic acid;
(R)-2-((1-(2-cyano-7-methyl-3-(1-methyl-6-oxo-1,6-dihydropyridin-3-yl)quinoxalin-5-yl)ethyl)amino)benzoic acid;
(R)-2-((1-(2-cyano-3-(4-fluorophenyl)-7-methylquinoxalin-5-yl)ethyl)amino)benzoic acid;
(R)-2-((1-(3-(4-chlorophenyl)-2-cyano-7-methylquinoxalin-5-yl)ethyl)amino)benzoic acid;
2-(((1R)-1-(2-cyano-7-methyl-3-((tetrahydrofuran-3-yl)thio)quinoxalin-5-yl)ethyl)amino)benzoic acid;
(R)-2-((1-(2-cyano-7-methyl-3-(((trifluoromethyl)sulfonyl)oxy)quinoxalin-5-yl)ethyl)amino)benzoic acid;
(R)-2-((1-(2-cyano-3-(((1-fluorocyclopropyl)methyl)amino)-7-methylquinoxalin-5-yl)ethyl)amino)benzoic acid;
(R)-2-((1-(2-cyano-7-methyl-3-(((1-(trifluoromethyl)cyclopropyl)methyl)amino)quinoxalin-5-yl)ethyl)amino)benzoic acid;
(R)-2-((1-(2-cyano-3-(((3,3-difluorocyclobutyl)methyl)amino)-7-methylquinoxalin-5-yl)ethyl)amino)benzoic acid;
2-(((1R)-1-(2-cyano-3-(((2,2-difluorocyclopropyl)methyl)amino)-7-methylquinoxalin-5-yl)ethyl)amino)benzoic acid;
2-(((1R)-1-(2-cyano-3-((1-cyclopropyl-2,2,2-trifluoroethyl)amino)-7-methylquinoxalin-5-yl)ethyl)amino)benzoic acid;
(R)-2-((1-(2-cyano-7-methyl-3-(3-(trifluoromethyl)-5,6-dihydro-[1,2,4]triazolo[4,3-a]pyrazin-7(8H)-yl)quinoxalin-5-yl)ethyl)amino)benzoic acid;
(R)-2-((1-(2-cyano-7-methyl-3-(2-(trifluoromethyl)-5,6-dihydroimidazo[1,2-a]pyrazin-7(8H)-yl)quinoxalin-5-yl)ethyl)amino)benzoic acid;
(R)-2-((1-(2-cyano-7-methyl-3-(4H-thieno[3,4-c]pyrrol-5(6H)-yl)quinoxalin-5-yl)ethyl)amino)benzoic acid;
(R)-2-((1-(2-cyano-3-(4-(2,2-difluoroethyl)piperazin-1-yl)-7-methylquinoxalin-5-yl)ethyl)amino)benzoic acid;
(R)-2-((1-(2-cyano-7-methyl-3-(4-(2,2,2-trifluoroethyl)piperazin-1-yl)quinoxalin-5-yl)ethyl)amino)benzoic acid;
(R)-2-((1-(2-cyano-3-(2,2-difluoro-7-azaspiro[3.5]nonan-7-yl)-7-methylquinoxalin-5-yl)ethyl)amino)benzoic acid;
2-(((1R)-1-(2-cyano-3-(2-(fluoromethyl)morpholino)-7-methylquinoxalin-5-yl)ethyl)amino)benzoic acid;
2-(((1R)-1-(2-cyano-3-(2-(2-fluoroethyl)morpholino)-7-methylquinoxalin-5-yl)ethyl)amino)benzoic acid;
2-(((1R)-1-(2-cyano-3-(5,5-difluorohexahydrocyclopenta[c]pyrrol-2(1H)-yl)-7-methylquinoxalin-5-yl)ethyl)amino)benzoic acid;
(R)-2-((1-(2-cyano-3-(2,2-difluoro-6-azaspiro[3.4]octan-6-yl)-7-methylquinoxalin-5-yl)ethyl)amino)benzoic acid;
(R)-2-((1-(2-cyano-3-(6,6-difluoro-2-azaspiro[3.3]heptan-2-yl)-7-methylquinoxalin-5-yl)ethyl)amino)benzoic acid;
2-(((1R)-1-(2-cyano-7-methyl-3-(2-(trifluoromethyl)morpholino)quinoxalin-5-yl)ethyl)amino)benzoic acid;
2-(((1R)-1-(2-cyano-3-(2-(fluoromethyl)azetidin-1-yl)-7-methylquinoxalin-5-yl)ethyl)amino)benzoic acid;
2-(((1R)-1-(2-cyano-3-(5-(2,2-difluoroethyl)-2,5-diazabicyclo[2.2.1]heptan-2-yl)-7-methylquinoxalin-5-yl)ethyl)amino)benzoic acid;
(R)-2-((1-(2-cyano-3-(4-fluoroisoindolin-2-yl)-7-methylquinoxalin-5-yl)ethyl)amino)benzoic acid;
(R)-2-((1-(2-cyano-7-methyl-3-(1-oxoisoindolin-2-yl)quinoxalin-5-yl)ethyl)amino)benzoic acid;
(R)-2-((1-(2-cyano-3-(5,6-difluoroisoindolin-2-yl)-7-methylquinoxalin-5-yl)ethyl)amino)benzoic acid;
(R)-2-((1-(2-cyano-7-methyl-3-(4-methyl-3-oxopiperazin-1-yl)quinoxalin-5-yl)ethyl)amino)benzoic acid;
(R)-2-((1-(2-cyano-7-methyl-3-(3-oxopiperazin-1-yl)quinoxalin-5-yl)ethyl)amino)benzoic acid;
2-(((1R)-1-(2-cyano-3-(3,3-difluoro-4-methoxypiperidin-1-yl)-7-methylquinoxalin-5-yl)ethyl)amino)benzoic acid;
2-(((1R)-1-(2-cyano-3-(3,3-difluoro-4-methylpiperidin-1-yl)-7-methylquinoxalin-5-yl)ethyl)amino)benzoic acid;
(R)-2-((1-(2-cyano-3-(4,4-difluoro-6-azaspiro[2.5]octan-6-yl)-7-methylquinoxalin-5-yl)ethyl)amino)benzoic acid;
2-(((1R)-1-(2-cyano-7-methyl-3-(3-(trifluoromethyl)piperidin-1-yl)quinoxalin-5-yl)ethyl)amino)benzoic acid;
(R)-2-((1-(2-cyano-3-(3,3-difluoropiperidin-1-yl)-7-methylquinoxalin-5-yl)ethyl)amino)benzoic acid;
2-(((1R)-1-(2-cyano-3-(4,4-difluoro-3-methoxypiperidin-1-yl)-7-methylquinoxalin-5-yl)ethyl)amino)benzoic acid;
2-(((1R)-1-(2-cyano-7-methyl-3-(3,4,4-trifluoropiperidin-1-yl)quinoxalin-5-yl)ethyl)amino)benzoic acid;
2-(((1R)-1-(2-cyano-3-(3,3-difluoro-4-methoxypyrrolidin-1-yl)-7-methylquinoxalin-5-yl)ethyl)amino)benzoic acid;
2-(((1R)-1-(2-cyano-3-(9,9-difluoro-3-oxa-7-azabicyclo[3.3.1]nonan-7-yl)-7-methylquinoxalin-5-yl)ethyl)amino)benzoic acid;
2-(((1R)-1-(2-cyano-3-(9,9-difluoro-3-azabicyclo[3.3.1]nonan-3-yl)-7-methylquinoxalin-5-yl)ethyl)amino)benzoic acid;
2-(((1R)-1-(2-cyano-3-(9,9-difluoro-1-(hydroxymethyl)-3-azabicyclo[3.3.1]nonan-3-yl)-7-methylquinoxalin-5-yl)ethyl)amino)benzoic acid;
2-(((1R)-1-(2-cyano-3-(8,8-difluoro-3-azabicyclo[3.2.1]octan-3-yl)-7-methylquinoxalin-5-yl)ethyl)amino)benzoic acid;
2-(((1R)-1-(2-cyano-3-(5,5-difluoro-2-azabicyclo[2.2.2]octan-2-yl)-7-methylquinoxalin-5-yl)ethyl)amino)benzoic acid;
2-(((1R)-1-(2-cyano-7-methyl-3-(7,7,9,9-tetrafluoro-3-azabicyclo[3.3.1]nonan-3-yl)quinoxalin-5-yl)ethyl)amino)benzoic acid;
2-(((1R)-1-(2-cyano-3-(6,6-difluoro-3-azabicyclo[3.1.1]heptan-3-yl)-7-methylquinoxalin-5-yl)ethyl)amino)benzoic acid;
2-(((1R)-1-(2-cyano-7-methyl-3-(1-(trifluoromethyl)-3-azabicyclo[3.1.0]hexan-3-yl)quinoxalin-5-yl)ethyl)amino)benzoic acid;
2-(((1R)-1-(2-cyano-3-(5,5-difluoro-2-azabicyclo[2.2.1]heptan-2-yl)-7-methylquinoxalin-5-yl)ethyl)amino)benzoic acid;
2-(((1R)-1-(2-cyano-3-(4,4-difluoro-3,5-dimethylpiperidin-1-yl)-7-methylquinoxalin-5-yl)ethyl)amino)benzoic acid;
(R)-2-((1-(2-cyano-3-(7,7-difluoro-5-azaspiro[2.4]heptan-5-yl)-7-methylquinoxalin-5-yl)ethyl)amino)benzoic acid;
2-(((1R)-1-(2-cyano-7-methyl-3-(6-(trifluoromethyl)-3-azabicyclo[3.1.0]hexan-3-yl)quinoxalin-5-yl)ethyl)amino)benzoic acid;
2-(((1R)-1-(2-cyano-3-(6,6-difluoro-3-azabicyclo[3.1.0]hexan-3-yl)-7-methylquinoxalin-5-yl)ethyl)amino)benzoic acid;
(R)-2-((1-(2-cyano-3-(6,7-dihydrothiazolo[4,5-c]pyridin-5(4H)-yl)-7-methylquinoxalin-5-yl)ethyl)amino)benzoic acid;
(R)-2-((1-(2-cyano-7-methyl-3-(2-methyl-6,7-dihydrothiazolo[4,5-c]pyridin-5(4H)-yl)quinoxalin-5-yl)ethyl)amino)benzoic acid;
(R)-2-((1-(2-cyano-3-(1,1-dioxidothiomorpholino)-7-methylquinoxalin-5-yl)ethyl)amino)benzoic acid;
(R)-2-((1-(2-cyano-7-methyl-3-(4-(thiazol-2-yl)piperazin-1-yl)quinoxalin-5-yl)ethyl)amino)benzoic acid;
(R)-2-((1-(2-cyano-7-methyl-3-(4-(5-(trifluoromethyl)pyridin-2-yl)piperazin-1-yl)quinoxalin-5-yl)ethyl)amino)benzoic acid;
(R)-2-((1-(2-cyano-7-methyl-3-(4-(5-(trifluoromethyl)benzo[d]thiazol-2-yl)piperazin-1-yl)quinoxalin-5-yl)ethyl)amino)benzoic acid;
(R)-2-((1-(2-cyano-7-methyl-3-(4-(4-methylthiazol-2-yl)piperazin-1-yl)quinoxalin-5-yl)ethyl)amino)benzoic acid;
(R)-2-((1-(2-cyano-7-methyl-3-(4-(1-methyl-3-(trifluoromethyl)-1H-pyrazol-4-yl)piperazin-1-yl)quinoxalin-5-yl)ethyl)amino)benzoic acid;
(R)-2-((1-(2-cyano-7-methyl-3-(4-(3-(trifluoromethyl)phenyl)piperazin-1-yl)quinoxalin-5-yl)ethyl)amino)benzoic acid;
(R)-2-((1-(2-cyano-3-(4-(2-fluorophenyl)piperazin-1-yl)-7-methylquinoxalin-5-yl)ethyl)amino)benzoic acid;
(R)-2-((1-(3-(4-(2-chlorophenyl)piperazin-1-yl)-2-cyano-7-methylquinoxalin-5-yl)ethyl)amino)benzoic acid;
(R)-2-((1-(2-cyano-3-(4-(2,4-difluorophenyl)piperazin-1-yl)-7-methylquinoxalin-5-yl)ethyl)amino)benzoic acid;
(R)-2-((1-(2-cyano-3-(4-(4-cyano-2-fluorophenyl)piperazin-1-yl)-7-methylquinoxalin-5-yl)ethyl)amino)benzoic acid;
(R)-2-((1-(2-cyano-7-methyl-3-(4-(4-(N-methylsulfamoyl)phenyl)piperazin-1-yl)quinoxalin-5-yl)ethyl)amino)benzoic acid;
(R)-2-((1-(2-cyano-3-(4-(4-fluorophenyl)piperazin-1-yl)-7-methylquinoxalin-5-yl)ethyl)amino)benzoic acid;
(R)-2-((1-(2-cyano-7-methyl-3-(4-(methylsulfonyl)piperazin-1-yl)quinoxalin-5-yl)ethyl)amino)benzoic acid;
(R)-2-((1-(3-(4-(4-chlorobenzoyl)piperazin-1-yl)-2-cyano-7-methylquinoxalin-5-yl)ethyl)amino)benzoic acid;
(R)-2-((1-(2-cyano-7-methyl-3-(4-(3-(trifluoromethyl)-1H-pyrazol-1-yl)piperidin-1-yl)quinoxalin-5-yl)ethyl)amino)benzoic acid;
(R)-2-((1-(2-cyano-7-methyl-3-(2-oxo-[1,4′-bipiperidin]-1′-yl)quinoxalin-5-yl)ethyl)amino)benzoic acid;
(R)-2-((1-(2-cyano-3-(4,4-difluoro-[1,4′-bipiperidin]-1′-yl)-7-methylquinoxalin-5-yl)ethyl)amino)benzoic acid;
(R)-2-((1-(2-cyano-7-methyl-3-(4-(trifluoromethyl)piperidin-1-yl)quinoxalin-5-yl)ethyl)amino)benzoic acid;
(R)-2-((1-(2-cyano-3-(1,1-difluoro-6-azaspiro[2.5]octan-6-yl)-7-methylquinoxalin-5-yl)ethyl)amino)benzoic acid;
2-(((1R)-1-(2-cyano-7-methyl-3-(7-methyl-6-oxo-2,7-diazaspiro[4.4]nonan-2-yl)quinoxalin-5-yl)ethyl)amino)benzoic acid;
2-(((1R)-1-(2-cyano-7-methyl-3-(2-oxo-1,7-diazaspiro[4.4]nonan-7-yl)quinoxalin-5-yl)ethyl)amino)benzoic acid;
2-(((1R)-1-(2-cyano-3-(7,7-difluoro-2-azaspiro[4.4]nonan-2-yl)-7-methylquinoxalin-5-yl)ethyl)amino)benzoic acid;
2-(((1R)-1-(2-cyano-7-methyl-3-(1,1,2,2-tetrafluoro-6-azaspiro[3.4]octan-6-yl)quinoxalin-5-yl)ethyl)amino)benzoic acid;
2-(((1R)-1-(2-cyano-3-(4,4-difluoro-3-(hydroxymethyl)piperidin-1-yl)-7-methylquinoxalin-5-yl)ethyl)amino)benzoic acid;
2-(((1R)-1-(2-cyano-7-methyl-3-(2-(trifluoromethyl)azetidin-1-yl)quinoxalin-5-yl)ethyl)amino)benzoic acid;
2-(((1R)-1-(2-cyano-3-(4-(2-cyano-4-(trifluoromethyl)phenyl)-2-methylpiperazin-1-yl)-7-methylquinoxalin-5-yl)ethyl)amino)benzoic acid;
2-(((1R)-1-(2-cyano-3-(4-(4-fluorophenyl)-2-methylpiperazin-1-yl)-7-methylquinoxalin-5-yl)ethyl)amino)benzoic acid;
2-(((1R)-1-(2-cyano-3-(4,4-difluoro-2-methylpiperidin-1-yl)-7-methylquinoxalin-5-yl)ethyl)amino)benzoic acid;
2-(((1R)-1-(2-cyano-3-(3,3-difluoro-2-methylpyrrolidin-1-yl)-7-methylquinoxalin-5-yl)ethyl)amino)benzoic acid;
(R)-2-((1-(2-cyano-7-methyl-3-(3,3,4,4-tetrafluoropyrrolidin-1-yl)quinoxalin-5-yl)ethyl)amino)benzoic acid;
2-(((1R)-1-(2-cyano-3-(3,5-difluoropiperidin-1-yl)-7-methylquinoxalin-5-yl)ethyl)amino)benzoic acid;
2-(((1R)-1-(2-cyano-3-(4-fluoro-2-(morpholine-4-carbonyl)pyrrolidin-1-yl)-7-methylquinoxalin-5-yl)ethyl)amino)benzoic acid;
2-(((1R)-1-(2-cyano-3-(6,6-difluoro-2-azabicyclo[2.2.1]heptan-2-yl)-7-methylquinoxalin-5-yl)ethyl)amino)benzoic acid;
(R)-2-((1-(2-cyano-3-(4,6-dihydro-5H-pyrrolo[3,4-d]thiazol-5-yl)-7-methylquinoxalin-5-yl)ethyl)amino)benzoic acid;
(R)-2-((1-(2-cyano-7-methyl-3-(2-methyl-4,6-dihydro-5H-pyrrolo[3,4-d]thiazol-5-yl)quinoxalin-5-yl)ethyl)amino)benzoic acid;
(R)-2-((1-(2-cyano-3-(5-fluoroisoindolin-2-yl)-7-methylquinoxalin-5-yl)ethyl)amino)benzoic acid;
(R)-2-((1-(2-cyano-3-(5-(difluoromethyl)isoindolin-2-yl)-7-methylquinoxalin-5-yl)ethyl)amino)benzoic acid;
(R)-2-((1-(2-cyano-7-methyl-3-(3-(5-(trifluoromethyl)isoxazol-3-yl)azetidin-1-yl)quinoxalin-5-yl)ethyl)amino)benzoic acid;
(R)-2-((1-(2-cyano-3-(3-fluoroazetidin-1-yl)-7-methylquinoxalin-5-yl)ethyl)amino)benzoic acid;
(R)-2-((1-(2-cyano-3-(3-(difluoromethyl)azetidin-1-yl)-7-methylquinoxalin-5-yl)ethyl)amino)benzoic acid;
(R)-2-((1-(2-cyano-7-methyl-3-(3-(trifluoromethyl)azetidin-1-yl)quinoxalin-5-yl)ethyl)amino)benzoic acid;
(R)-2-((1-(2-cyano-3-(5,5-difluoro-2-azaspiro[3.3]heptan-2-yl)-7-methylquinoxalin-5-yl)ethyl)amino)benzoic acid;
(R)-2-((1-(2-cyano-3-(1,1-difluoro-5-azaspiro[2.3]hexan-5-yl)-7-methylquinoxalin-5-yl)ethyl)amino)benzoic acid;
2-(((1R)-1-(3-(7-chloro-3-(hydroxymethyl)-3,4-dihydroisoquinolin-2(1H)-yl)-2-cyano-7-methylquinoxalin-5-yl)ethyl)amino)benzoic acid;
2-(((1R)-1-(2-cyano-3-(3,3-difluoro-2-methylazetidin-1-yl)-7-methylquinoxalin-5-yl)ethyl)amino)benzoic acid;
2-(((1R)-1-(3-((1-benzyl-2-oxopiperidin-3-yl)(methyl)amino)-2-cyano-7-methylquinoxalin-5-yl)ethyl)amino)benzoic acid;
2-(((1R)-1-(2-cyano-7-methyl-3-(methyl(4,5,6,7-tetrahydrobenzo[d]thiazol-6-yl)amino)quinoxalin-5-yl)ethyl)amino)benzoic acid;
(R)-2-((1-(2-cyano-3-((3,3-difluorocyclobutyl)(methyl)amino)-7-methylquinoxalin-5-yl)ethyl)amino)benzoic acid;
2-(((1R)-1-(2-cyano-7-methyl-3-(3-methyl-4-oxo-3,9-diazabicyclo[4.2.1]nonan-9-yl)quinoxalin-5-yl)ethyl)amino)benzoic acid;
2-(((1R)-1-(2-cyano-3-(3,3-difluoro-8-azabicyclo[3.2.1]octan-8-yl)-7-methylquinoxalin-5-yl)ethyl)amino)benzoic acid;
(R)-2-((1-(3-chloro-2-cyano-7-methylquinoxalin-5-yl)ethyl)amino)benzoic acid;
(R)-2-((1-(2-cyano-7-methyl-3-phenoxyquinoxalin-5-yl)ethyl)amino)benzoic acid;
(R)-2-((1-(2-cyano-3-(3-cyanophenoxy)-7-methylquinoxalin-5-yl)ethyl)amino)benzoic acid;
(R)-2-((1-(3-(benzyloxy)-2-cyano-7-methylquinoxalin-5-yl)ethyl)amino)benzoic acid;
2-(((1R)-1-(2-cyano-7-methyl-3-((tetrahydrofuran-3-yl)oxy)quinoxalin-5-yl)ethyl)amino)benzoic acid;
(R)-2-((1-(2-cyano-3-methoxy-7-methylquinoxalin-5-yl)ethyl)amino)benzoic acid;
(R)-2-((1-(2-cyano-3-(4-(ethoxymethyl)piperidin-1-yl)-7-methylquinoxalin-5-yl)ethyl)amino)benzoic acid;
(R)-2-((1-(2-cyano-3-(diethylamino)-7-methylquinoxalin-5-yl)ethyl)amino)benzoic acid;
2-(((1R)-1-(2-cyano-7-methyl-3-((1-(pyridin-3-yl)ethyl)amino)quinoxalin-5-yl)ethyl)amino)benzoic acid;
2-(((1R)-1-(2-cyano-7-methyl-3-((3,3,3-trifluoro-2-methylpropyl)amino)quinoxalin-5-yl)ethyl)amino)benzoic acid;
2-(((1R)-1-(2-cyano-3-((1-cyclopropylethyl)amino)-7-methylquinoxalin-5-yl)ethyl)amino)benzoic acid;
(R)-2-((1-(2-cyano-3-(isobutylamino)-7-methylquinoxalin-5-yl)ethyl)amino)benzoic acid;
2-(((1R)-1-(2-cyano-3-(3-hydroxy-3-isopropylpyrrolidin-1-yl)-7-methylquinoxalin-5-yl)ethyl)amino)benzoic acid;
2-(((1R)-1-(2-cyano-3-(1-isopropyl-7,7-dioxido-7-thia-2-azaspiro[3.5]nonan-2-yl)-7-methylquinoxalin-5-yl)ethyl)amino)benzoic acid;
(R)-3-((1-(3-(4-(bicyclo[1.1.1]pentan-1-yl-3-d)piperidin-1-yl)-2-cyano-7-methylquinoxalin-5-yl)ethyl)amino)-6-chloropicolinic acid;
(R)-6-chloro-3-((1-(2-cyano-3-((3-cyanophenyl)amino)-7-methylquinoxalin-5-yl)ethyl)amino)picolinic acid;
6-chloro-3-(((1R)-1-(2-cyano-7-methyl-3-(((tetrahydrofuran-3-yl)methyl)amino)quinoxalin-5-yl)ethyl)amino)picolinic acid;
(R)-6-chloro-3-((1-(2-cyano-3-(((3,3-difluorocyclobutyl)methyl)amino)-7-methylquinoxalin-5-yl)ethyl)amino)picolinic acid;
(R)-6-chloro-3-((1-(2-cyano-3-((2-cyanoallyl)amino)-7-methylquinoxalin-5-yl)ethyl)amino)picolinic acid;
(R)-6-chloro-3-((1-(2-cyano-3-(cyclobutylamino)-7-methylquinoxalin-5-yl)ethyl)amino)picolinic acid;
(R)-6-chloro-3-((1-(2-cyano-3-((3-(hydroxymethyl)bicyclo[1.1.1]pentan-1-yl)amino)-7-methylquinoxalin-5-yl)ethyl)amino)picolinic acid;
6-chloro-3-(((1R)-1-(2-cyano-3-((1-cyclopropyl-2,2,2-trifluoroethyl)amino)-7-methylquinoxalin-5-yl)ethyl)amino)picolinic acid;
(R)-6-chloro-3-((1-(2-cyano-7-methyl-3-(3-(trifluoromethyl)-5,6-dihydro-[1,2,4]triazolo[4,3-a]pyrazin-7(8H)-yl)quinoxalin-5-yl)ethyl)amino)picolinic acid;
(R)-6-chloro-3-((1-(2-cyano-3-(5,6-dihydroimidazo[1,2-a]pyrazin-7(8H)-yl)-7-methylquinoxalin-5-yl)ethyl)amino)picolinic acid;
(R)-6-chloro-3-((1-(2-cyano-3-(2,3-dimethyl-5,6-dihydroimidazo[1,2-a]pyrazin-7(8H)-yl)-7-methylquinoxalin-5-yl)ethyl)amino)picolinic acid;
(R)-6-chloro-3-((1-(2-cyano-7-methyl-3-(2-(trifluoromethyl)-5,6-dihydroimidazo[1,2-a]pyrazin-7(8H)-yl)quinoxalin-5-yl)ethyl)amino)picolinic acid;
(R)-6-chloro-3-((1-(2-cyano-7-methyl-3-(4H-thieno[3,4-c]pyrrol-5(6H)-yl)quinoxalin-5-yl)ethyl)amino)picolinic acid;
(R)-6-chloro-3-((1-(2-cyano-3-(5,6-dihydroimidazo[1,5-a]pyrazin-7(8H)-yl)-7-methylquinoxalin-5-yl)ethyl)amino)picolinic acid;
(R)-6-chloro-3-((1-(2-cyano-3-(6,7-dihydropyrazolo[1,5-a]pyrazin-5(4H)-yl)-7-methylquinoxalin-5-yl)ethyl)amino)picolinic acid;
(R)-6-chloro-3-((1-(2-cyano-7-methyl-3-(piperazin-1-yl)quinoxalin-5-yl)ethyl)amino)picolinic acid;
(R)-6-chloro-3-((1-(2-cyano-3-(4-(2,2-difluoroethyl)piperazin-1-yl)-7-methylquinoxalin-5-yl)ethyl)amino)picolinic acid;
(R)-6-chloro-3-((1-(2-cyano-7-methyl-3-(4-(2,2,2-trifluoroethyl)piperazin-1-yl)quinoxalin-5-yl)ethyl)amino)picolinic acid;
(R)-6-chloro-3-((1-(2-cyano-7-methyl-3-(4-methylpiperazin-1-yl)quinoxalin-5-yl)ethyl)amino)picolinic acid;
(R)-3-((1-(3-(azepan-1-yl)-2-cyano-7-methylquinoxalin-5-yl)ethyl)amino)-6-chloropicolinic acid;
(R)-6-chloro-3-((1-(2-cyano-7-methyl-3-(piperidin-1-yl)quinoxalin-5-yl)ethyl)amino)picolinic acid;
(R)-6-chloro-3-((1-(2-cyano-7-methyl-3-(2-oxa-8-azaspiro[4.5]decan-8-yl)quinoxalin-5-yl)ethyl)amino)picolinic acid;
(R)-6-chloro-3-((1-(2-cyano-7-methyl-3-(6-azaspiro[2.5]octan-6-yl)quinoxalin-5-yl)ethyl)amino)picolinic acid;
(R)-3-((1-(3-(azetidin-1-yl)-2-cyano-7-methylquinoxalin-5-yl)ethyl)amino)-6-chloropicolinic acid;
6-chloro-3-(((1R)-1-(2-cyano-3-(2-hydroxy-8-azaspiro[4.5]decan-8-yl)-7-methylquinoxalin-5-yl)ethyl)amino)picolinic acid;
(R)-6-chloro-3-((1-(2-cyano-3-(2,2-difluoro-7-azaspiro[3.5]nonan-7-yl)-7-methylquinoxalin-5-yl)ethyl)amino)picolinic acid;
(R)-6-chloro-3-((1-(2-cyano-7-methyl-3-(4-methylpiperidin-1-yl)quinoxalin-5-yl)ethyl)amino)picolinic acid;
6-chloro-3-(((1R)-1-(2-cyano-7-methyl-3-(octahydro-2H-4,7-ethanoisoindol-2-yl)quinoxalin-5-yl)ethyl)amino)picolinic acid;
6-chloro-3-(((1R)-1-(2-cyano-3-(6,6-dimethyl-3-azabicyclo[3.2.0]heptan-3-yl)-7-methylquinoxalin-5-yl)ethyl)amino)picolinic acid;
(R)-6-chloro-3-((1-(2-cyano-7-methyl-3-(2-azaspiro[3.3]heptan-2-yl)quinoxalin-5-yl)ethyl)amino)picolinic acid;
(R)-6-chloro-3-((1-(2-cyano-3-(2-azadispiro[3.1.36.14]decan-2-yl)-7-methylquinoxalin-5-yl)ethyl)amino)picolinic acid;
6-chloro-3-(((1R)-1-(2-cyano-7-methyl-3-(5-methylhexahydropyrrolo[3,4-c]pyrrol-2(1H)-yl)quinoxalin-5-yl)ethyl)amino)picolinic acid;
6-chloro-3-(((1R)-1-(2-cyano-3-(2-(fluoromethyl)morpholino)-7-methylquinoxalin-5-yl)ethyl)amino)picolinic acid;
6-chloro-3-(((1R)-1-(2-cyano-3-(2-(2-fluoroethyl)morpholino)-7-methylquinoxalin-5-yl)ethyl)amino)picolinic acid;
6-chloro-3-(((1R)-1-(2-cyano-3-(5,5-difluorohexahydrocyclopenta[c]pyrrol-2(1H)-yl)-7-methylquinoxalin-5-yl)ethyl)amino)picolinic acid;
6-chloro-3-(((1R)-1-(2-cyano-3-(2-cyclopropylmorpholino)-7-methylquinoxalin-5-yl)ethyl)amino)picolinic acid;
6-chloro-3-(((1R)-1-(2-cyano-3-(2-cyclopropyl-6-methylmorpholino)-7-methylquinoxalin-5-yl)ethyl)amino)picolinic acid;
(R)-3-((1-(3-(3-azabicyclo[3.1.1]heptan-3-yl)-2-cyano-7-methylquinoxalin-5-yl)ethyl)amino)-6-chloropicolinic acid;
3-(((1R)-1-(3-(3-azabicyclo[3.1.0]hexan-3-yl)-2-cyano-7-methylquinoxalin-5-yl)ethyl)amino)-6-chloropicolinic acid;
(R)-6-chloro-3-((1-(2-cyano-3-(6-(4-cyanophenyl)-2,6-diazaspiro[3.3]heptan-2-yl)-7-methylquinoxalin-5-yl)ethyl)amino)picolinic acid;
(R)-6-chloro-3-((1-(2-cyano-3-(2,2-difluoro-6-azaspiro[3.4]octan-6-yl)-7-methylquinoxalin-5-yl)ethyl)amino)picolinic acid;
(R)-6-chloro-3-((1-(2-cyano-3-(6,6-difluoro-2-azaspiro[3.3]heptan-2-yl)-7-methylquinoxalin-5-yl)ethyl)amino)picolinic acid;
6-chloro-3-(((1R)-1-(2-cyano-3-(2-cyclopropyl-2-methylmorpholino)-7-methylquinoxalin-5-yl)ethyl)amino)picolinic acid;
3-(((1R)-1-(3-(3,6-diazabicyclo[3.1.1]heptan-6-yl)-2-cyano-7-methylquinoxalin-5-yl)ethyl)amino)-6-chloropicolinic acid;
(R)-3-((1-(3-(7-azabicyclo[2.2.1]heptan-7-yl)-2-cyano-7-methylquinoxalin-5-yl)ethyl)amino)-6-chloropicolinic acid;
(R)-3-((1-(3-(2-azaadamantan-2-yl)-2-cyano-7-methylquinoxalin-5-yl)ethyl)amino)-6-chloropicolinic acid;
(R)-6-chloro-3-((1-(2-cyano-3-(1-(methoxymethyl)-4-methyl-2-azabicyclo[2.1.1]hexan-2-yl)-7-methylquinoxalin-5-yl)ethyl)amino)picolinic acid;
6-chloro-3-(((1R)-1-(2-cyano-3-(8-((dimethylamino)methyl)-6-oxa-9-azaspiro[4.5]decan-9-yl)-7-methylquinoxalin-5-yl)ethyl)amino)picolinic acid;
6-chloro-3-(((1R)-1-(2-cyano-3-(2-(fluoromethyl)azetidin-1-yl)-7-methylquinoxalin-5-yl)ethyl)amino)picolinic acid;
(R)-3-((1-(3-(2-azabicyclo[2.2.2]octan-2-yl)-2-cyano-7-methylquinoxalin-5-yl)ethyl)amino)-6-chloropicolinic acid;
3-(((1R)-1-(3-(2-azabicyclo[2.2.1]heptan-2-yl)-2-cyano-7-methylquinoxalin-5-yl)ethyl)amino)-6-chloropicolinic acid;
6-chloro-3-(((1R)-1-(2-cyano-3-(hexahydro-3,5-methanocyclopenta[b]pyrrol-1(2H)-yl)-7-methylquinoxalin-5-yl)ethyl)amino)picolinic acid;
6-chloro-3-(((1R)-1-(2-cyano-3-(5-(2,2-difluoroethyl)-2,5-diazabicyclo[2.2.1]heptan-2-yl)-7-methylquinoxalin-5-yl)ethyl)amino)picolinic acid;
6-chloro-3-(((1R)-1-(2-cyano-3-(1-(hydroxymethyl)-6-azabicyclo[3.2.1]octan-6-yl)-7-methylquinoxalin-5-yl)ethyl)amino)picolinic acid;
(R)-3-((1-(3-(2-azabicyclo[2.1.1]hexan-2-yl)-2-cyano-7-methylquinoxalin-5-yl)ethyl)amino)-6-chloropicolinic acid;
6-chloro-3-(((1R)-1-(2-cyano-7-methyl-3-(2-methyl-4-(pyridin-3-ylmethyl)piperazin-1-yl)quinoxalin-5-yl)ethyl)amino)picolinic acid;
6-chloro-3-(((1R)-1-(2-cyano-7-methyl-3-(2-methylazetidin-1-yl)quinoxalin-5-yl)ethyl)amino)picolinic acid;
6-chloro-3-(((1R)-1-(2-cyano-3-(2,3-dimethylmorpholino)-7-methylquinoxalin-5-yl)ethyl)amino)picolinic acid;
(R)-6-chloro-3-((1-(2-cyano-7-methyl-3-(1-oxoisoindolin-2-yl)quinoxalin-5-yl)ethyl)amino)picolinic acid;
(R)-6-chloro-3-((1-(2-cyano-7-methyl-3-(4-methyl-3-oxopiperazin-1-yl)quinoxalin-5-yl)ethyl)amino)picolinic acid;
(R)-6-chloro-3-((1-(2-cyano-7-methyl-3-(3-oxopiperazin-1-yl)quinoxalin-5-yl)ethyl)amino)picolinic acid;
6-chloro-3-(((1R)-1-(2-cyano-3-(3,3-difluoro-4-methoxypiperidin-1-yl)-7-methylquinoxalin-5-yl)ethyl)amino)picolinic acid;
6-chloro-3-(((1R)-1-(2-cyano-3-(3-hydroxy-2-(2-methoxyphenyl)pyrrolidin-1-yl)-7-methylquinoxalin-5-yl)ethyl)amino)picolinic acid;
6-chloro-3-(((1R)-1-(2-cyano-3-(3,3-difluoro-4-methylpiperidin-1-yl)-7-methylquinoxalin-5-yl)ethyl)amino)picolinic acid;
(R)-6-chloro-3-((1-(2-cyano-3-(3,4-dihydroquinolin-1(2H)-yl)-7-methylquinoxalin-5-yl)ethyl)amino)picolinic acid;
6-chloro-3-(((1R)-1-(2-cyano-3-(3-hydroxypiperidin-1-yl)-7-methylquinoxalin-5-yl)ethyl)amino)picolinic acid;
6-chloro-3-(((1R)-1-(2-cyano-3-(3-hydroxy-3-methylpiperidin-1-yl)-7-methylquinoxalin-5-yl)ethyl)amino)picolinic acid;
6-chloro-3-(((1R)-1-(2-cyano-3-(2-(4-hydroxy-4-methyltetrahydro-2H-pyran-3-yl)pyrrolidin-1-yl)-7-methylquinoxalin-5-yl)ethyl)amino)picolinic acid;
6-chloro-3-(((1R)-1-(2-cyano-3-(4,4-difluoro-3-methoxypiperidin-1-yl)-7-methylquinoxalin-5-yl)ethyl)amino)picolinic acid;
6-chloro-3-(((1R)-1-(2-cyano-7-methyl-3-(3,4,4-trifluoropiperidin-1-yl)quinoxalin-5-yl)ethyl)amino)picolinic acid;
6-chloro-3-(((1R)-1-(2-cyano-3-(9,9-difluoro-3-oxa-7-azabicyclo[3.3.1]nonan-7-yl)-7-methylquinoxalin-5-yl)ethyl)amino)picolinic acid;
6-chloro-3-(((1R)-1-(2-cyano-3-(9,9-difluoro-3-azabicyclo[3.3.1]nonan-3-yl)-7-methylquinoxalin-5-yl)ethyl)amino)picolinic acid;
6-chloro-3-(((1R)-1-(2-cyano-3-(9,9-difluoro-1-(hydroxymethyl)-3-azabicyclo[3.3.1]nonan-3-yl)-7-methylquinoxalin-5-yl)ethyl)amino)picolinic acid;
6-chloro-3-(((1R)-1-(2-cyano-3-(8-hydroxy-3-azabicyclo[3.2.1]octan-3-yl)-7-methylquinoxalin-5-yl)ethyl)amino)picolinic acid;
6-chloro-3-(((1R)-1-(2-cyano-3-(8,8-difluoro-3-azabicyclo[3.2.1]octan-3-yl)-7-methylquinoxalin-5-yl)ethyl)amino)picolinic acid;
6-chloro-3-(((1R)-1-(2-cyano-3-(5-hydroxy-2-azabicyclo[2.2.2]octan-2-yl)-7-methylquinoxalin-5-yl)ethyl)amino)picolinic acid;
6-chloro-3-(((1R)-1-(2-cyano-3-(5,5-difluoro-2-azabicyclo[2.2.2]octan-2-yl)-7-methylquinoxalin-5-yl)ethyl)amino)picolinic acid;
6-chloro-3-(((1R)-1-(2-cyano-7-methyl-3-(7,7,9,9-tetrafluoro-3-azabicyclo[3.3.1]nonan-3-yl)quinoxalin-5-yl)ethyl)amino)picolinic acid;
6-chloro-3-(((1R)-1-(2-cyano-3-(6-hydroxyhexahydro-3,5-methanocyclopenta[b]pyrrol-1(2H)-yl)-7-methylquinoxalin-5-yl)ethyl)amino)picolinic acid;
6-chloro-3-(((1R)-1-(2-cyano-3-(6-(4-cyanophenyl)-3,6-diazabicyclo[3.1.1]heptan-3-yl)-7-methylquinoxalin-5-yl)ethyl)amino)picolinic acid;
6-chloro-3-(((1R)-1-(2-cyano-3-(6-hydroxy-6-methyl-3-azabicyclo[3.1.1]heptan-3-yl)-7-methylquinoxalin-5-yl)ethyl)amino)picolinic acid;
6-chloro-3-(((1R)-1-(2-cyano-3-(5-(4-cyanophenyl)-2,5-diazabicyclo[2.2.1]heptan-2-yl)-7-methylquinoxalin-5-yl)ethyl)amino)picolinic acid;
6-chloro-3-(((1R)-1-(2-cyano-7-methyl-3-(5-phenyl-2-azabicyclo[2.2.1]heptan-2-yl)quinoxalin-5-yl)ethyl)amino)picolinic acid;
6-chloro-3-(((1R)-1-(2-cyano-3-(4,4-difluoro-3,5-dimethylpiperidin-1-yl)-7-methylquinoxalin-5-yl)ethyl)amino)picolinic acid;
3-(((1R)-1-(3-(7-(tert-butyl)-6,7-dihydropyrazolo[1,5-a]pyrimidin-4(5H)-yl)-2-cyano-7-methylquinoxalin-5-yl)ethyl)amino)-6-chloropicolinic acid;
(R)-6-chloro-3-((1-(2-cyano-3-(6,7-dihydrothiazolo[4,5-c]pyridin-5(4H)-yl)-7-methylquinoxalin-5-yl)ethyl)amino)picolinic acid;
(R)-6-chloro-3-((1-(2-cyano-7-methyl-3-(2-methyl-6,7-dihydrothiazolo[4,5-c]pyridin-5(4H)-yl)quinoxalin-5-yl)ethyl)amino)picolinic acid;
(R)-6-chloro-3-((1-(2-cyano-7-methyl-3-(1-methyl-1,4,5,7-tetrahydro-6H-pyrazolo[3,4-c]pyridin-6-yl)quinoxalin-5-yl)ethyl)amino)picolinic acid;
(R)-6-chloro-3-((1-(2-cyano-7-methyl-3-(5,6,8,9-tetrahydro-7H-imidazo[1,2-d][1,4]diazepin-7-yl)quinoxalin-5-yl)ethyl)amino)picolinic acid;
(R)-6-chloro-3-((1-(2-cyano-3-(1,1-dioxidothiomorpholino)-7-methylquinoxalin-5-yl)ethyl)amino)picolinic acid;
(R)-6-chloro-3-((1-(2-cyano-7-methyl-3-(4-(thiazol-2-yl)piperazin-1-yl)quinoxalin-5-yl)ethyl)amino)picolinic acid;
(R)-6-chloro-3-((1-(2-cyano-7-methyl-3-(4-(pyridin-2-yl)piperazin-1-yl)quinoxalin-5-yl)ethyl)amino)picolinic acid;
(R)-6-chloro-3-((1-(2-cyano-7-methyl-3-(4-(5-(trifluoromethyl)benzo[d]thiazol-2-yl)piperazin-1-yl)quinoxalin-5-yl)ethyl)amino)picolinic acid;
(R)-6-chloro-3-((1-(2-cyano-7-methyl-3-(4-(4-methylthiazol-2-yl)piperazin-1-yl)quinoxalin-5-yl)ethyl)amino)picolinic acid;
(R)-6-chloro-3-((1-(2-cyano-7-methyl-3-(4-(pyridin-3-yl)piperazin-1-yl)quinoxalin-5-yl)ethyl)amino)picolinic acid;
(R)-6-chloro-3-((1-(2-cyano-7-methyl-3-(4-(1-methyl-3-(trifluoromethyl)-1H-pyrazol-4-yl)piperazin-1-yl)quinoxalin-5-yl)ethyl)amino)picolinic acid;
(R)-6-chloro-3-((1-(2-cyano-3-(4-(3-cyano-1-methyl-1H-pyrazol-4-yl)piperazin-1-yl)-7-methylquinoxalin-5-yl)ethyl)amino)picolinic acid;
(R)-6-chloro-3-((1-(2-cyano-7-methyl-3-(4-(1-methyl-1H-pyrazol-4-yl)piperazin-1-yl)quinoxalin-5-yl)ethyl)amino)picolinic acid;
(R)-6-chloro-3-((1-(2-cyano-7-methyl-3-(4-(pyridin-4-yl)piperazin-1-yl)quinoxalin-5-yl)ethyl)amino)picolinic acid;
(R)-6-chloro-3-((1-(2-cyano-7-methyl-3-(4-phenylpiperazin-1-yl)quinoxalin-5-yl)ethyl)amino)picolinic acid;
(R)-3-((1-(3-(4-(4-(aminomethyl)phenyl)piperazin-1-yl)-2-cyano-7-methylquinoxalin-5-yl)ethyl)amino)-6-chloropicolinic acid;
(R)-6-chloro-3-((1-(2-cyano-7-methyl-3-(4-(3-(trifluoromethyl)phenyl)piperazin-1-yl)quinoxalin-5-yl)ethyl)amino)picolinic acid;
(R)-6-chloro-3-((1-(2-cyano-3-(4-(3-cyanophenyl)piperazin-1-yl)-7-methylquinoxalin-5-yl)ethyl)amino)picolinic acid;
(R)-6-chloro-3-((1-(2-cyano-3-(4-(2,6-dimethylphenyl)piperazin-1-yl)-7-methylquinoxalin-5-yl)ethyl)amino)picolinic acid;
(R)-6-chloro-3-((1-(2-cyano-7-methyl-3-(4-(o-tolyl)piperazin-1-yl)quinoxalin-5-yl)ethyl)amino)picolinic acid;
(R)-6-chloro-3-((1-(2-cyano-3-(4-(3-cyano-2-methylphenyl)piperazin-1-yl)-7-methylquinoxalin-5-yl)ethyl)amino)picolinic acid;
(R)-6-chloro-3-((1-(2-cyano-3-(4-(2-cyanopyridin-3-yl)piperazin-1-yl)-7-methylquinoxalin-5-yl)ethyl)amino)picolinic acid;
(R)-6-chloro-3-((1-(2-cyano-3-(4-(5-cyanopyrimidin-2-yl)piperazin-1-yl)-7-methylquinoxalin-5-yl)ethyl)amino)picolinic acid;
(R)-6-chloro-3-((1-(2-cyano-3-(4-(3-cyano-1-methyl-1H-indazol-7-yl)piperazin-1-yl)-7-methylquinoxalin-5-yl)ethyl)amino)picolinic acid;
(R)-6-chloro-3-((1-(2-cyano-3-(4-(2-fluorophenyl)piperazin-1-yl)-7-methylquinoxalin-5-yl)ethyl)amino)picolinic acid;
(R)-6-chloro-3-((1-(3-(4-(2-chlorophenyl)piperazin-1-yl)-2-cyano-7-methylquinoxalin-5-yl)ethyl)amino)picolinic acid;
(R)-6-chloro-3-((1-(2-cyano-3-(4-(2-cyanophenyl)piperazin-1-yl)-7-methylquinoxalin-5-yl)ethyl)amino)picolinic acid;
(R)-6-chloro-3-((1-(2-cyano-3-(4-(2,4-difluorophenyl)piperazin-1-yl)-7-methylquinoxalin-5-yl)ethyl)amino)picolinic acid;
(R)-6-chloro-3-((1-(2-cyano-3-(4-(4-cyano-2-fluorophenyl)piperazin-1-yl)-7-methylquinoxalin-5-yl)ethyl)amino)picolinic acid;
(R)-6-chloro-3-((1-(2-cyano-7-methyl-3-(4-(4-(N-methylsulfamoyl)phenyl)piperazin-1-yl)quinoxalin-5-yl)ethyl)amino)picolinic acid;
(R)-6-chloro-3-((1-(2-cyano-3-(4-(4-fluorophenyl)piperazin-1-yl)-7-methylquinoxalin-5-yl)ethyl)amino)picolinic acid;
(R)-6-chloro-3-((1-(2-cyano-7-methyl-3-(4-(4-(methylcarbamoyl)phenyl)piperazin-1-yl)quinoxalin-5-yl)ethyl)amino)picolinic acid;
(R)-3-((1-(3-(4-(4-carbamoylphenyl)piperazin-1-yl)-2-cyano-7-methylquinoxalin-5-yl)ethyl)amino)-6-chloropicolinic acid;
(R)-6-chloro-3-((1-(2-cyano-3-(4-(4-cyanophenyl)piperazin-1-yl)-7-methylquinoxalin-5-yl)ethyl)amino)picolinic acid;
(R)-6-chloro-3-((1-(2-cyano-3-(4-(4-cyano-2-methylphenyl)piperazin-1-yl)-7-methylquinoxalin-5-yl)ethyl)amino)picolinic acid;
(R)-6-chloro-3-((1-(2-cyano-3-(4-(8-cyanoisoquinolin-5-yl)piperazin-1-yl)-7-methylquinoxalin-5-yl)ethyl)amino)picolinic acid;
(R)-6-chloro-3-((1-(2-cyano-3-(4-(4-cyano-7-hydroxynaphthalen-1-yl)piperazin-1-yl)-7-methylquinoxalin-5-yl)ethyl)amino)picolinic acid;
(R)-6-chloro-3-((1-(2-cyano-3-(4-(4-cyano-6-hydroxynaphthalen-1-yl)piperazin-1-yl)-7-methylquinoxalin-5-yl)ethyl)amino)picolinic acid;
(R)-6-chloro-3-((1-(2-cyano-3-(4-(4,6-dicyanonaphthalen-1-yl)piperazin-1-yl)-7-methylquinoxalin-5-yl)ethyl)amino)picolinic acid;
(R)-6-chloro-3-((1-(2-cyano-7-methyl-3-(4-(methylsulfonyl)piperazin-1-yl)quinoxalin-5-yl)ethyl)amino)picolinic acid;
(R)-6-chloro-3-((1-(2-cyano-7-methyl-3-(4-(oxetan-3-yl)piperazin-1-yl)quinoxalin-5-yl)ethyl)amino)picolinic acid;
(R)-6-chloro-3-((1-(2-cyano-7-methyl-3-(4-(tetrahydro-2H-pyran-4-yl)piperazin-1-yl)quinoxalin-5-yl)ethyl)amino)picolinic acid;
(R)-6-chloro-3-((1-(2-cyano-3-(4-cyclopropylpiperazin-1-yl)-7-methylquinoxalin-5-yl)ethyl)amino)picolinic acid;
(R)-6-chloro-3-((1-(2-cyano-7-methyl-3-(4-(1-methyl-1H-pyrazole-4-carbonyl)piperazin-1-yl)quinoxalin-5-yl)ethyl)amino)picolinic acid;
(R)-3-((1-(3-(4-benzoylpiperazin-1-yl)-2-cyano-7-methylquinoxalin-5-yl)ethyl)amino)-6-chloropicolinic acid;
(R)-6-chloro-3-((1-(3-(4-(4-chlorobenzoyl)piperazin-1-yl)-2-cyano-7-methylquinoxalin-5-yl)ethyl)amino)picolinic acid;
(R)-6-chloro-3-((1-(2-cyano-3-(4-(cyclopentanecarbonyl)piperazin-1-yl)-7-methylquinoxalin-5-yl)ethyl)amino)picolinic acid;
(R)-6-chloro-3-((1-(2-cyano-3-(4-(cyclopropanecarbonyl)piperazin-1-yl)-7-methylquinoxalin-5-yl)ethyl)amino)picolinic acid;
(R)-6-chloro-3-((1-(2-cyano-7-methyl-3-(4-(3-(trifluoromethyl)-1H-pyrazol-1-yl)piperidin-1-yl)quinoxalin-5-yl)ethyl)amino)picolinic acid;
(R)-6-chloro-3-((1-(2-cyano-7-methyl-3-(4-(1-methyl-1H-pyrazol-3-yl)piperidin-1-yl)quinoxalin-5-yl)ethyl)amino)picolinic acid;
(R)-3-((1-(3-(4-(1H-pyrazol-3-yl)piperidin-1-yl)-2-cyano-7-methylquinoxalin-5-yl)ethyl)amino)-6-chloropicolinic acid;
(R)-6-chloro-3-((1-(2-cyano-3-(4-(4-methoxyphenyl)piperidin-1-yl)-7-methylquinoxalin-5-yl)ethyl)amino)picolinic acid;
(R)-6-chloro-3-((1-(2-cyano-3-(4-methoxypiperidin-1-yl)-7-methylquinoxalin-5-yl)ethyl)amino)picolinic acid;
(R)-6-chloro-3-((1-(2-cyano-3-(4-hydroxypiperidin-1-yl)-7-methylquinoxalin-5-yl)ethyl)amino)picolinic acid;
(R)-3-((1-(3-([1,4′-bipiperidin]-1′-yl)-2-cyano-7-methylquinoxalin-5-yl)ethyl)amino)-6-chloropicolinic acid;
(R)-6-chloro-3-((1-(2-cyano-3-(4,4-difluoro-[1,4′-bipiperidin]-1′-yl)-7-methylquinoxalin-5-yl)ethyl)amino)picolinic acid;
(R)-6-chloro-3-((1-(2-cyano-3-(4-(dimethylamino)piperidin-1-yl)-7-methylquinoxalin-5-yl)ethyl)amino)picolinic acid;
(R)-6-chloro-3-((1-(2-cyano-3-(4-cyclopentylpiperidin-1-yl)-7-methylquinoxalin-5-yl)ethyl)amino)picolinic acid;
(R)-6-chloro-3-((1-(2-cyano-3-(4-cyclobutylpiperidin-1-yl)-7-methylquinoxalin-5-yl)ethyl)amino)picolinic acid;
(R)-6-chloro-3-((1-(2-cyano-7-methyl-3-(4-(trifluoromethyl)piperidin-1-yl)quinoxalin-5-yl)ethyl)amino)picolinic acid;
(R)-6-chloro-3-((1-(2-cyano-3-(4-cyanopiperidin-1-yl)-7-methylquinoxalin-5-yl)ethyl)amino)picolinic acid;
(R)-6-chloro-3-((1-(2-cyano-7-methyl-3-(1-oxa-8-azaspiro[4.5]decan-8-yl)quinoxalin-5-yl)ethyl)amino)picolinic acid;
(R)-6-chloro-3-((1-(2-cyano-3-(1,1-difluoro-6-azaspiro[2.5]octan-6-yl)-7-methylquinoxalin-5-yl)ethyl)amino)picolinic acid;
(R)-6-chloro-3-((1-(2-cyano-3-(4-methoxy-4-methylpiperidin-1-yl)-7-methylquinoxalin-5-yl)ethyl)amino)picolinic acid;
(R)-6-chloro-3-((1-(2-cyano-3-(4-cyano-4-methylpiperidin-1-yl)-7-methylquinoxalin-5-yl)ethyl)amino)picolinic acid;
6-chloro-3-(((1R)-1-(2-cyano-7-methyl-3-(3-(pyridin-2-yl)pyrrolidin-1-yl)quinoxalin-5-yl)ethyl)amino)picolinic acid;
6-chloro-3-(((1R)-1-(2-cyano-7-methyl-3-(3-(pyridin-4-yl)pyrrolidin-1-yl)quinoxalin-5-yl)ethyl)amino)picolinic acid;
6-chloro-3-(((1R)-1-(2-cyano-7-methyl-3-(3-phenylpyrrolidin-1-yl)quinoxalin-5-yl)ethyl)amino)picolinic acid;
6-chloro-3-(((1R)-1-(2-cyano-3-(3-(1,3-dihydroxypropan-2-yl)pyrrolidin-1-yl)-7-methylquinoxalin-5-yl)ethyl)amino)picolinic acid;
6-chloro-3-(((1R)-1-(2-cyano-7-methyl-3-(1-oxa-6-azaspiro[3.4]octan-6-yl)quinoxalin-5-yl)ethyl)amino)picolinic acid;
6-chloro-3-(((1R)-1-(2-cyano-7-methyl-3-(spiro[bicyclo[2.2.1]heptane-2,3′-pyrrolidin]-1′-yl)quinoxalin-5-yl)ethyl)amino)picolinic acid;
6-chloro-3-(((1R)-1-(2-cyano-7-methyl-3-(7-methyl-6-oxo-2,7-diazaspiro[4.4]nonan-2-yl)quinoxalin-5-yl)ethyl)amino)picolinic acid;
6-chloro-3-(((1R)-1-(2-cyano-3-(8,9-dihydroxy-6-oxa-2-azaspiro[4.5]decan-2-yl)-7-methylquinoxalin-5-yl)ethyl)amino)picolinic acid;
6-chloro-3-(((1R)-1-(2-cyano-7-methyl-3-(2-oxo-1,7-diazaspiro[4.4]nonan-7-yl)quinoxalin-5-yl)ethyl)amino)picolinic acid;
6-chloro-3-(((1R)-1-(2-cyano-3-(7,7-difluoro-2-azaspiro[4.4]nonan-2-yl)-7-methylquinoxalin-5-yl)ethyl)amino)picolinic acid;
6-chloro-3-(((1R)-1-(2-cyano-7-methyl-3-(1,1,2,2-tetrafluoro-6-azaspiro[3.4]octan-6-yl)quinoxalin-5-yl)ethyl)amino)picolinic acid;
6-chloro-3-(((1R)-1-(2-cyano-3-(4-(4-cyanophenyl)-3-methylpiperazin-1-yl)-7-methylquinoxalin-5-yl)ethyl)amino)picolinic acid;
6-chloro-3-(((1R)-1-(2-cyano-7-methyl-3-(2-(trifluoromethyl)azetidin-1-yl)quinoxalin-5-yl)ethyl)amino)picolinic acid;
6-chloro-3-(((1R)-1-(2-cyano-7-methyl-3-(2-methyl-4-phenylpiperazin-1-yl)quinoxalin-5-yl)ethyl)amino)picolinic acid;
6-chloro-3-(((1R)-1-(2-cyano-3-(4-(3-cyanophenyl)-2-methylpiperazin-1-yl)-7-methylquinoxalin-5-yl)ethyl)amino)picolinic acid;
6-chloro-3-(((1R)-1-(2-cyano-3-(4-(2-cyanophenyl)-2-methylpiperazin-1-yl)-7-methylquinoxalin-5-yl)ethyl)amino)picolinic acid;
6-chloro-3-(((1R)-1-(2-cyano-3-(4-(2-cyano-4-(trifluoromethyl)phenyl)-2-methylpiperazin-1-yl)-7-methylquinoxalin-5-yl)ethyl)amino)picolinic acid;
6-chloro-3-(((1R)-1-(2-cyano-3-(4-(4-fluorophenyl)-2-methylpiperazin-1-yl)-7-methylquinoxalin-5-yl)ethyl)amino)picolinic acid;
6-chloro-3-(((1R)-1-(2-cyano-3-(4-(4-cyanophenyl)-2-methylpiperazin-1-yl)-7-methylquinoxalin-5-yl)ethyl)amino)picolinic acid;
6-chloro-3-(((1R)-1-(2-cyano-3-(4-(2-cyano-5-methylphenyl)-2-methylpiperazin-1-yl)-7-methylquinoxalin-5-yl)ethyl)amino)picolinic acid;
6-chloro-3-(((1R)-1-(2-cyano-3-(4,4-difluoro-2-methylpiperidin-1-yl)-7-methylquinoxalin-5-yl)ethyl)amino)picolinic acid;
(R)-6-chloro-3-((1-(2-cyano-7-methyl-3-(3,3,4,4-tetrafluoropyrrolidin-1-yl)quinoxalin-5-yl)ethyl)amino)picolinic acid;
6-chloro-3-(((1R)-1-(2-cyano-3-(6-hydroxy-2-azabicyclo[2.2.2]octan-2-yl)-7-methylquinoxalin-5-yl)ethyl)amino)picolinic acid;
6-chloro-3-(((1R)-1-(2-cyano-3-(4-fluoro-2-(morpholine-4-carbonyl)pyrrolidin-1-yl)-7-methylquinoxalin-5-yl)ethyl)amino)picolinic acid;
6-chloro-3-(((1R)-1-(2-cyano-7-methyl-3-(1,2,3,4-tetrahydro-1,4-epiminonaphthalen-9-yl)quinoxalin-5-yl)ethyl)amino)picolinic acid;
(R)-6-chloro-3-((1-(2-cyano-3-(4,6-dihydro-5H-pyrrolo[3,4-d]thiazol-5-yl)-7-methylquinoxalin-5-yl)ethyl)amino)picolinic acid;
(R)-6-chloro-3-((1-(2-cyano-3-(5,7-dihydro-6H-pyrrolo[3,4-b]pyrazin-6-yl)-7-methylquinoxalin-5-yl)ethyl)amino)picolinic acid;
(R)-6-chloro-3-((1-(2-cyano-7-methyl-3-(2-methyl-4,6-dihydro-5H-pyrrolo[3,4-d]thiazol-5-yl)quinoxalin-5-yl)ethyl)amino)picolinic acid;
(R)-6-chloro-3-((1-(2-cyano-3-(1,3-dihydro-2H-pyrrolo[3,4-c]pyridin-2-yl)-7-methylquinoxalin-5-yl)ethyl)amino)picolinic acid;
(R)-6-chloro-3-((1-(2-cyano-3-(5,7-dihydro-6H-pyrrolo[3,4-b]pyridin-6-yl)-7-methylquinoxalin-5-yl)ethyl)amino)picolinic acid;
(R)-6-chloro-3-((1-(2-cyano-3-(4-fluoroisoindolin-2-yl)-7-methylquinoxalin-5-yl)ethyl)amino)picolinic acid;
(R)-6-chloro-3-((1-(2-cyano-3-(4-cyanoisoindolin-2-yl)-7-methylquinoxalin-5-yl)ethyl)amino)picolinic acid;
(R)-6-chloro-3-((1-(2-cyano-3-(isoindolin-2-yl)-7-methylquinoxalin-5-yl)ethyl)amino)picolinic acid;
(R)-6-chloro-3-((1-(2-cyano-3-(5-(hydroxymethyl)isoindolin-2-yl)-7-methylquinoxalin-5-yl)ethyl)amino)picolinic acid;
(R)-6-chloro-3-((1-(2-cyano-7-methyl-3-(5-methylisoindolin-2-yl)quinoxalin-5-yl)ethyl)amino)picolinic acid;
(R)-6-chloro-3-((1-(2-cyano-3-(5-(difluoromethyl)isoindolin-2-yl)-7-methylquinoxalin-5-yl)ethyl)amino)picolinic acid;
(R)-6-chloro-3-((1-(2-cyano-3-(5,6-dimethoxyisoindolin-2-yl)-7-methylquinoxalin-5-yl)ethyl)amino)picolinic acid;
(R)-6-chloro-3-((1-(2-cyano-3-(5,6-difluoroisoindolin-2-yl)-7-methylquinoxalin-5-yl)ethyl)amino)picolinic acid;
6-chloro-3-(((1R)-1-(2-cyano-7-methyl-3-(tetrahydro-1H,3H-spiro[pyrrolo[2,1-c][1,4]oxazine-4,5′-[1,3]oxazinan]-3′-yl)quinoxalin-5-yl)ethyl)amino)picolinic acid;
6-chloro-3-(((1R)-1-(2-cyano-3-(decahydro-2H-cycloocta[c]pyrrol-2-yl)-7-methylquinoxalin-5-yl)ethyl)amino)picolinic acid;
6-chloro-3-(((1R)-1-(2-cyano-7-methyl-3-(8-azatricyclo[4.3.0.02,5]nonan-8-yl)quinoxalin-5-yl)ethyl)amino)picolinic acid;
(R)-3-((1-(3-(1H,3H,4H-3a,6a-(methanooxymethano)furo[3,4-c]pyrrol-5(6H)-yl)-2-cyano-7-methylquinoxalin-5-yl)ethyl)amino)-6-chloropicolinic acid;
6-chloro-3-(((1R)-1-(2-cyano-3-(8-hydroxy-6-azaspiro[3.4]octan-6-yl)-7-methylquinoxalin-5-yl)ethyl)amino)picolinic acid;
(R)-6-chloro-3-((1-(2-cyano-7-methyl-3-(3-(5-(trifluoromethyl)isoxazol-3-yl)azetidin-1-yl)quinoxalin-5-yl)ethyl)amino)picolinic acid;
(R)-6-chloro-3-((1-(2-cyano-7-methyl-3-(3-(pyridin-2-yl)azetidin-1-yl)quinoxalin-5-yl)ethyl)amino)picolinic acid;
(R)-6-chloro-3-((1-(2-cyano-7-methyl-3-(3-(pyridin-3-yl)azetidin-1-yl)quinoxalin-5-yl)ethyl)amino)picolinic acid;
(R)-6-chloro-3-((1-(2-cyano-7-methyl-3-(3-phenylazetidin-1-yl)quinoxalin-5-yl)ethyl)amino)picolinic acid;
(R)-6-chloro-3-((1-(2-cyano-3-(3-methoxyazetidin-1-yl)-7-methylquinoxalin-5-yl)ethyl)amino)picolinic acid;
(R)-6-chloro-3-((1-(2-cyano-3-(3-fluoroazetidin-1-yl)-7-methylquinoxalin-5-yl)ethyl)amino)picolinic acid;
(R)-6-chloro-3-((1-(2-cyano-3-(3-(difluoromethyl)azetidin-1-yl)-7-methylquinoxalin-5-yl)ethyl)amino)picolinic acid;
(R)-6-chloro-3-((1-(2-cyano-3-(3-cyanoazetidin-1-yl)-7-methylquinoxalin-5-yl)ethyl)amino)picolinic acid;
(R)-6-chloro-3-((1-(2-cyano-3-(7-(4-cyanophenyl)-2,7-diazaspiro[3.5]nonan-2-yl)-7-methylquinoxalin-5-yl)ethyl)amino)picolinic acid;
6-chloro-3-(((1R)-1-(2-cyano-3-(8-hydroxyoctahydro-2H-4,7-methanoisoindol-2-yl)-7-methylquinoxalin-5-yl)ethyl)amino)picolinic acid;
6-chloro-3-(((1R)-1-(2-cyano-7-methyl-3-(5-methyl-2-azabicyclo[2.2.1]heptan-2-yl)quinoxalin-5-yl)ethyl)amino)picolinic acid;
6-chloro-3-(((1R)-1-(2-cyano-7-methyl-3-(1′-methyltetrahydrospiro[furo[3,4-b]pyrrole-3,4′-piperidin]-1(2H)-yl)quinoxalin-5-yl)ethyl)amino)picolinic acid;
6-chloro-3-(((1R)-1-(2-cyano-3-(1-(hydroxymethyl)isoindolin-2-yl)-7-methylquinoxalin-5-yl)ethyl)amino)picolinic acid;
6-chloro-3-(((1R)-1-(3-(7-chloro-3-(hydroxymethyl)-3,4-dihydroisoquinolin-2(1H)-yl)-2-cyano-7-methylquinoxalin-5-yl)ethyl)amino)picolinic acid;
6-chloro-3-(((1R)-1-(2-cyano-3-(3-hydroxyoctahydro-1H-indol-1-yl)-7-methylquinoxalin-5-yl)ethyl)amino)picolinic acid;
6-chloro-3-(((1R)-1-(2-cyano-7-methyl-3-(1-methyloctahydro-2H-isoindol-2-yl)quinoxalin-5-yl)ethyl)amino)picolinic acid;
6-chloro-3-(((1R)-1-(2-cyano-3-(3,3-difluoro-2-methylazetidin-1-yl)-7-methylquinoxalin-5-yl)ethyl)amino)picolinic acid;
(R)-6-chloro-3-((1-(2-cyano-7-methyl-3-(methyl((5,6,8,9-tetrahydro-[1,2,4]triazolo[4,3-d][1,4]oxazepin-3-yl)methyl)amino)quinoxalin-5-yl)ethyl)amino)picolinic acid;
6-chloro-3-(((1R)-1-(2-cyano-7-methyl-3-(methyl((5,6,7,8-tetrahydroimidazo[1,2-a]pyridin-7-yl)methyl)amino)quinoxalin-5-yl)ethyl)amino)picolinic acid;
(R)-6-chloro-3-((1-(2-cyano-3-((cyclopropylmethyl)(methyl)amino)-7-methylquinoxalin-5-yl)ethyl)amino)picolinic acid;
6-chloro-3-(((1R)-1-(2-cyano-3-(((1-hydroxy-2,3-dihydro-1H-inden-1-yl)methyl)(methyl)amino)-7-methylquinoxalin-5-yl)ethyl)amino)picolinic acid;
(R)-6-chloro-3-((1-(2-cyano-3-(dimethylamino)-7-methylquinoxalin-5-yl)ethyl)amino)picolinic acid;
3-(((1R)-1-(3-((1-benzyl-2-oxopiperidin-3-yl)(methyl)amino)-2-cyano-7-methylquinoxalin-5-yl)ethyl)amino)-6-chloropicolinic acid;
6-chloro-3-(((1R)-1-(2-cyano-7-methyl-3-(methyl(4,5,6,7-tetrahydrobenzo[d]thiazol-6-yl)amino)quinoxalin-5-yl)ethyl)amino)picolinic acid;
(R)-6-chloro-3-((1-(2-cyano-3-((3,3-difluorocyclobutyl)(methyl)amino)-7-methylquinoxalin-5-yl)ethyl)amino)picolinic acid;
6-chloro-3-(((1R)-1-(2-cyano-7-methyl-3-(methyl(1-methyl-2,3-dihydro-1H-inden-2-yl)amino)quinoxalin-5-yl)ethyl)amino)picolinic acid;
6-chloro-3-(((1R)-1-(2-cyano-3-((2-methoxy-2,3-dihydro-1H-inden-1-yl)(methyl)amino)-7-methylquinoxalin-5-yl)ethyl)amino)picolinic acid;
6-chloro-3-(((1R)-1-(2-cyano-3-(3-(4-cyanophenyl)-3,8-diazabicyclo[3.2.1]octan-8-yl)-7-methylquinoxalin-5-yl)ethyl)amino)picolinic acid;
6-chloro-3-(((1R)-1-(2-cyano-7-methyl-3-(3-methyl-4-oxo-3,9-diazabicyclo[4.2.1]nonan-9-yl)quinoxalin-5-yl)ethyl)amino)picolinic acid;
6-chloro-3-(((1R)-1-(2-cyano-3-(3,3-difluoro-8-azabicyclo[3.2.1]octan-8-yl)-7-methylquinoxalin-5-yl)ethyl)amino)picolinic acid;
6-chloro-3-(((1R)-1-(2-cyano-3-(3-(4-cyanophenyl)-3,6-diazabicyclo[3.1.1]heptan-6-yl)-7-methylquinoxalin-5-yl)ethyl)amino)picolinic acid;
(R)-6-chloro-3-((1-(2-cyano-7-methyl-3-(piperidin-4-yl)quinoxalin-5-yl)ethyl)amino)picolinic acid;
(R)-6-chloro-3-((1-(2-cyano-7-methyl-3-(1-methylpiperidin-4-yl)quinoxalin-5-yl)ethyl)amino)picolinic acid;
(R)-6-chloro-3-((1-(2-cyano-3-(3,6-dihydro-2H-pyran-4-yl)-7-methylquinoxalin-5-yl)ethyl)amino)picolinic acid;
(R)-6-chloro-3-((1-(2-cyano-7-methyl-3-(1,2,3,6-tetrahydropyridin-4-yl)quinoxalin-5-yl)ethyl)amino)picolinic acid;
(R)-6-chloro-3-((1-(2-cyano-7-methyl-3-(1-methyl-1,2,3,6-tetrahydropyridin-4-yl)quinoxalin-5-yl)ethyl)amino)picolinic acid;
(R)-6-chloro-3-((1-(2-cyano-3,7-dimethylquinoxalin-5-yl)ethyl)amino)picolinic acid;
6-chloro-3-(((1R)-1-(2-cyano-7-methyl-3-(1-methyl-2-oxopiperidin-4-yl)quinoxalin-5-yl)ethyl)amino)picolinic acid;
(R)-6-chloro-3-((1-(2-cyano-3-(1-(4-cyanophenyl)piperidin-4-yl)-7-methylquinoxalin-5-yl)ethyl)amino)picolinic acid;
(R)-3-((1-(3-(1-acetylpiperidin-4-yl)-2-cyano-7-methylquinoxalin-5-yl)ethyl)amino)-6-chloropicolinic acid;
(R)-6-chloro-3-((1-(2-cyano-3-(4,4-difluorocyclohexyl)-7-methylquinoxalin-5-yl)ethyl)amino)picolinic acid;
(R)-3-((1-(3-(1-acetyl-1,2,3,6-tetrahydropyridin-4-yl)-2-cyano-7-methylquinoxalin-5-yl)ethyl)amino)-6-chloropicolinic acid;
(R)-6-chloro-3-((1-(2-cyano-3-((4-cyanophenyl)ethynyl)-7-methylquinoxalin-5-yl)ethyl)amino)picolinic acid;
(R)-6-chloro-3-((1-(2-cyano-7-methyl-3-(1-methyl-1H-pyrazol-3-yl)quinoxalin-5-yl)ethyl)amino)picolinic acid;
(R)-6-chloro-3-((1-(2-cyano-7-methyl-3-(pyridin-2-yl)quinoxalin-5-yl)ethyl)amino)picolinic acid;
(R)-6-chloro-3-((1-(2-cyano-7-methyl-3-(pyridin-3-yl)quinoxalin-5-yl)ethyl)amino)picolinic acid;
(R)-6-chloro-3-((1-(2-cyano-7-methyl-3-(1-methyl-1H-pyrazol-4-yl)quinoxalin-5-yl)ethyl)amino)picolinic acid;
(R)-6-chloro-3-((1-(2-cyano-7-methyl-3-(1-methyl-1H-imidazol-4-yl)quinoxalin-5-yl)ethyl)amino)picolinic acid;
(R)-6-chloro-3-((1-(2-cyano-3-(1-(4-cyanophenyl)-1H-pyrazol-4-yl)-7-methylquinoxalin-5-yl)ethyl)amino)picolinic acid;
(R)-6-chloro-3-((1-(2-cyano-7-methyl-3-(pyridin-4-yl)quinoxalin-5-yl)ethyl)amino)picolinic acid;
(R)-6-chloro-3-((1-(2-cyano-7-methyl-3-phenylquinoxalin-5-yl)ethyl)amino)picolinic acid;
(R)-6-chloro-3-((1-(2-cyano-7-methyl-3-(p-tolyl)quinoxalin-5-yl)ethyl)amino)picolinic acid;
(R)-6-chloro-3-((1-(2-cyano-3-(3-methoxyphenyl)-7-methylquinoxalin-5-yl)ethyl)amino)picolinic acid;
(R)-6-chloro-3-((1-(2-cyano-3-(3-(2-cyanopropan-2-yl)phenyl)-7-methylquinoxalin-5-yl)ethyl)amino)picolinic acid;
(R)-6-chloro-3-((1-(2-cyano-3-(3-cyanophenyl)-7-methylquinoxalin-5-yl)ethyl)amino)picolinic acid;
(R)-6-chloro-3-((1-(2-cyano-7-methyl-3-(1H-pyrazol-4-yl)quinoxalin-5-yl)ethyl)amino)picolinic acid;
(R)-6-chloro-3-((1-(2-cyano-3-(1H-imidazol-4-yl)-7-methylquinoxalin-5-yl)ethyl)amino)picolinic acid;
(R)-6-chloro-3-((1-(2-cyano-7-methyl-3-(o-tolyl)quinoxalin-5-yl)ethyl)amino)picolinic acid;
(R)-6-chloro-3-((1-(2-cyano-3-(1-(4-cyanophenyl)-1H-imidazol-4-yl)-7-methylquinoxalin-5-yl)ethyl)amino)picolinic acid;
(R)-6-chloro-3-((1-(2-cyano-3-(2-methoxyphenyl)-7-methylquinoxalin-5-yl)ethyl)amino)picolinic acid;
(R)-6-chloro-3-((1-(2-cyano-3-(2-cyanophenyl)-7-methylquinoxalin-5-yl)ethyl)amino)picolinic acid;
(R)-6-chloro-3-((1-(2-cyano-3-(4-methoxyphenyl)-7-methylquinoxalin-5-yl)ethyl)amino)picolinic acid;
(R)-6-chloro-3-((1-(2-cyano-3-(4-fluorophenyl)-7-methylquinoxalin-5-yl)ethyl)amino)picolinic acid;
(R)-6-chloro-3-((1-(3-(4-chlorophenyl)-2-cyano-7-methylquinoxalin-5-yl)ethyl)amino)picolinic acid;
(R)-6-chloro-3-((1-(2-cyano-3-(4-cyanophenyl)-7-methylquinoxalin-5-yl)ethyl)amino)picolinic acid;
(R)-6-chloro-3-((1-(3-chloro-2-cyano-7-methylquinoxalin-5-yl)ethyl)amino)picolinic acid;
6-chloro-3-(((1R)-1-(2-cyano-7-methyl-3-((tetrahydrofuran-3-yl)thio)quinoxalin-5-yl)ethyl)amino)picolinic acid;
(R)-6-chloro-3-((1-(2-cyano-7-methyl-3-phenoxyquinoxalin-5-yl)ethyl)amino)picolinic acid;
(R)-6-chloro-3-((1-(2-cyano-3-(3-cyanophenoxy)-7-methylquinoxalin-5-yl)ethyl)amino)picolinic acid;
(R)-6-chloro-3-((1-(2-cyano-7-methyl-3-(((trifluoromethyl)sulfonyl)oxy)quinoxalin-5-yl)ethyl)amino)picolinic acid;
(R)-3-((1-(3-(benzyloxy)-2-cyano-7-methylquinoxalin-5-yl)ethyl)amino)-6-chloropicolinic acid;
6-chloro-3-(((1R)-1-(2-cyano-7-methyl-3-((tetrahydrofuran-3-yl)oxy)quinoxalin-5-yl)ethyl)amino)picolinic acid;
(R)-6-chloro-3-((1-(2-cyano-3-methoxy-7-methylquinoxalin-5-yl)ethyl)amino)picolinic acid;
(R)-6-chloro-3-((1-(2-cyano-3-hydroxy-7-methylquinoxalin-5-yl)ethyl)amino)picolinic acid;
(R)-6-chloro-3-((1-(2-cyano-3-(diethylamino)-7-methylquinoxalin-5-yl)ethyl)amino)picolinic acid;
6-chloro-3-(((1R)-1-(2-cyano-7-methyl-3-((1-(pyridin-3-yl)ethyl)amino)quinoxalin-5-yl)ethyl)amino)picolinic acid;
6-chloro-3-(((1R)-1-(2-cyano-7-methyl-3-((3,3,3-trifluoro-2-methylpropyl)amino)quinoxalin-5-yl)ethyl)amino)picolinic acid;
6-chloro-3-(((1R)-1-(2-cyano-3-((1-cyclopropylethyl)amino)-7-methylquinoxalin-5-yl)ethyl)amino)picolinic acid;
(R)-6-chloro-3-((1-(2-cyano-3-(isobutylamino)-7-methylquinoxalin-5-yl)ethyl)amino)picolinic acid;
6-chloro-3-(((1R)-1-(2-cyano-3-(3-hydroxy-3-isopropylpyrrolidin-1-yl)-7-methylquinoxalin-5-yl)ethyl)amino)picolinic acid;
6-chloro-3-(((1R)-1-(2-cyano-3-(1-isopropyl-7,7-dioxido-7-thia-2-azaspiro[3.5]nonan-2-yl)-7-methylquinoxalin-5-yl)ethyl)amino)picolinic acid; or
a pharmaceutically acceptable salt thereof.

68. A pharmaceutical composition comprising a compound or salt according to claim 1 together with a pharmaceutically acceptable carrier, excipient or diluent.

69. A method of treating a disease or disorder associated with modulation of phosphoinositide 3-kinase (PI3K), comprising administering to a patient in need thereof a therapeutically effective amount of a compound of claim 1 or a pharmaceutical composition comprising a compound or salt according to claim 1 together with a pharmaceutically acceptable carrier, excipient or diluent.

70. (canceled)

71. The method of claim 69, wherein the PI3K associated with the disease or disorder has a H1047R mutation.

72. The method of claim 69, wherein the disease or disorder is a cancer.

73. (canceled)

74. (canceled)

75. (canceled)

76. A method of treating cancer or a disorder, the method comprising administering to a patient in need thereof a therapeutically effective amount of a compound of claim 1 or a pharmaceutical composition comprising a compound or salt according to claim 1 together with a pharmaceutically acceptable carrier, excipient or diluent.

77. (canceled)

78. A method of treating cancer comprising administering a compound according to claim 1 or a pharmaceutical composition comprising a compound or salt according to claim 1 together with a pharmaceutically acceptable carrier, excipient or diluent and a KRAS inhibitor to a patient in need thereof, wherein the cancer is breast cancer, uterine carcinosarcoma, uterine endometrial carcinoma, colorectal adenocarcinoma, stomach adenocarcinoma, head and neck squamous cell carcinoma, cholangiocarcinoma, esophageal adenocarcinoma, bladder carcinoma, lung squamous cell carcinoma, brain glioma, adrenocortical carcinoma, liver hepatocellular carcinoma, sarcoma, prostate adenocarcinoma, kidney renal cell carcinoma, lung adenocarcinoma, ovarian cystadenocarcinoma, glioblastoma multiforme, melanoma.

79. (canceled)

80. (canceled)
